(19) United States
(12) Patent Application Publication
(10) Pub. No.: US 2023/0210853 A1
(43) Pub. Date: Jul. 6, 2023

(54) TARGETED NEK7 INHIBITION FOR MODULATION OF THE NLRP3 INFLAMMASOME

(71) Applicant: Halia Therapeutics, Inc., Salt Lake City, UT (US)

(72) Inventors: David James BEARSS, Salt Lake City, UT (US); John Sai Keong KAUWE, III, Salt Lake City, UT (US); Alexis Henri Abel MOLLARD, Lehi, UT (US)

(21) Appl. No.: 17/998,135

(22) PCT Filed: May 7, 2021
(86) PCT No.: PCT/US2021/031426

§ 371 Date: Nov. 7, 2022

Related U.S. Application Data
(60) Provisional application No. 63/022,159, filed on May 8, 2020.
(60) Provisional application No. 63/036,244, filed on Jun. 8, 2020.
(60) Provisional application No. 63/141,370, filed on Jan. 25, 2021.
(60) Provisional application No. 63/167,523, filed on Mar. 29, 2021.
(60) Provisional application No. 63/170,776, filed on Apr. 5, 2021.
(60) Provisional application No. 63/170,761, filed on Apr. 5, 2021.
(60) Provisional application No. 63/170,708, filed on Apr. 5, 2021.
(60) Provisional application No. 63/178,385, filed on Apr. 22, 2021.
(60) Provisional application No. 63/185,257, filed on May 6, 2021.
(60) Provisional application No. 63/185,267, filed on May 6, 2021.
(60) Provisional application No. 63/185,274, filed on May 6, 2021.
(60) Provisional application No. 63/185,282, filed on May 6, 2021.
(60) Provisional application No. 63/185,286, filed on May 6, 2021.

(51) Int. Cl. A61K 31/519; A61K 31/437

(52) U.S. Cl. CPC A61K 31/519; A61K 31/437

(57) ABSTRACT

Compositions comprising at least one NEK7 protein and a NEK7 small molecule inhibitor compound comprising at least one of the following features: (i) a hinge-binding element comprising at least one hydrogen donor and at least one hydrogen acceptor, (ii) a flexible linker, (iii) a urea linker, or (iv) a hydrophobic back pocket group. Methods associated with preparation and use of such NEK7 inhibitors, pharmaceutical compositions comprising such NEK7 inhibitors and methods to prevent diseases or disorders (e.g., via modulation of NLRP3 inflammasome activity) are also provided.

### BACKGROUND

### Technical Field

Embodiments of the present disclosure are generally directed to compositions and methods for their preparation and use as therapeutic or prophylactic agents, for example for treatment of inflammation.

### Description of the Related Art

Inflammasomes are multi-protein complexes whose activation plays a central role in innate immunity and inflammation. To date, four inflammasomes have been described. NLRP1, NLRC4, NLRP3, and ALM2. The NLRP3 inflammasome is composed of NLRP3, ASC, and caspase-1. Its activation results in the activation of caspase-1 which promotes the secretion of IL-1β and IL-18, cytokines that mediate inflammation in animal disease models of several autoimmune diseases, myocardial infarction, metabolic syndromes, inflammatory bowel disease, and macrophage activation syndrome.

NEK7 is a member of the family of NIMA-related kinases (NEKs) that act as NLRP3-binding proteins to regulate its oligomerization and activation NEK7 is a serine/threonine kinase essential for mitotic entry, cell cycle progression, cell division, and mitotic progression. It is expressed in a variety of tissues such as the brain, heart, lung, liver, and spleen. Overexpression of NEK7 induces the production of abnormal cells, which has an intimate connection to tumors, such as retinoblastoma, gallbladder cancer and carcinoma of the head and neck.

A great number of inhibitors have been widely used to disturb effector signaling pathways, involving IL-1β or IL-18 without abolishing the inflammation response. Inhibitors of NLRP3 inflammasome activation that block the NLRP3-NEK7 interaction can have therapeutic or prophylactic activity in several human diseases, such as type 2 diabetes (T2D), atherosclerosis, gout, and neurodegenerative diseases. However, the exact mechanism of the NLRP3-NEK7 interaction is not well understood.

Accordingly, there is a need to develop inhibitors that will directly target NEK7 to affect the inflammatory response modulated by the NLRP3 inflammasome in several pathological diseases, such as gout, atherosclerosis, Type 2 diabetes, metabolic syndrome, macular degeneration. Alzheimer's disease, multiple sclerosis, and inflammatory bowel disease. Embodiments of the present disclosure fulfill this need and provide further related advantages.

### BRIEF SUMMARY

In brief, embodiments of the present disclosure provide compounds, including pharmaceutically acceptable salts, stereoisomers and prodrugs thereof, which are capable of modulating the activity of the NLRP3 inflammasome via type-2 inhibition of NEK7.

In one aspect, the disclosure provides compositions comprising at least one NEK7 protein and a NEK7 small molecule inhibitor compound comprising at least one of the following features: (i) a hinge-binding element comprising at least one hydrogen donor and at least one hydrogen acceptor, (ii) a flexible linker, (iii) a urea linker, or (iv) a hydrophobic back pocket group.

In another aspect, methods of treating or preventing a disease or disorder, the methods comprising administering a NEK7 inhibitor to a subject in need thereof, the NEK7 inhibitor comprising at least one of the following features: (i) a hinge-binding element comprising at least one hydrogen donor and at least one hydrogen acceptor, (ii) a flexible linker, (iii) a urea linker, or (iv) a hydrophobic back pocket group, wherein the disease or disorder is a NLRP3-mediated disorder, are also provided.

### BRIEF DESCRIPTION OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements, and have been solely selected for ease of recognition in the figures.

FIG. 1 shows a small molecule compound that exhibits a type 1.5 binding mode illustrating interactions with (i) hinge residues (ALA116 and ALA114) via a pair of hydrogen bonds with the aminopyrimidine core (dashed lines); (ii) ASP179 of the activation loop (part of the conserved DFG motif which is occasionally replaced with DLG as is the case with NEK7) via a hydrogen bond with the indoline amide carbonyl (dashed lines), stabilizing the loop in the “in” position and restricting access to the allosteric back pocket. FIG. 1 shows that no interactions are observed between the small molecule compound and the αC helix that is positioned outward in a catalytically inactive conformation (not shown).

FIG. 2 shows a NEK7 small molecule inhibitor compound that exhibits a type 2 binding mode illustrating interactions with (i) a hinge residue (ALA114) via a pair of hydrogen bonds with the azaindole core (dashed lines); (ii) ASP179 of the DLG motif of the activation loop via a hydrogen bond with the urea carbonyl (dashed lines), stabilizing the loop in the “out” position and allowing access to the allosteric back pocket which is occupied by a bulky hydrophobic group; (iii) GLU82 of the αC helix via a pair of hydrogen bonds with the two NH groups of the urea linker (dashed lines), stabilizing the helix in an inward position.

FIG. 3A and FIG. 3B shows a side-by-side comparison of type 1.5 (FIG. 3A) versus type 2 (FIG. 3B) binding mode, illustration the similarities and differences between the binding mode, namely (i) similar donor-acceptor pairs in the hinge binding region; (ii) different conformations of the activation loop caused by interactions of the ASP179 residue of the DLG motif with the compounds, resulting in an inaccessible allosteric pocket for the type 1.5 binding mode (as indicated by the angular pose adopted by the compound to avoid steric clashes with residue of the activation loop past the DLG motif) and a fully occupied allosteric pocket for the type 2 binding mode (in which the residues of the activation loop past the DLG motif are not creating any steric hindrance); and (iii) different orientations of the αC helix which is positioned outward for the Type 1.5 binding mode and inward in the Type 2 binding mode.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, +5% or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Oxo” refers to the ═O substituent.

“NEK7 small molecule inhibitor compound” refers to an organic molecule (i.e., comprising carbon) having a molecular weight ranging from about 300 g/mole to about 1500 g/mol. In embodiments, the NEK7 small molecule inhibitor compound comprises carbon, nitrogen, and one or more halogen atoms. In embodiments, the NEK7 small molecule inhibitor compound comprises carbon, oxygen, nitrogen, and one or more halogen atoms. In some embodiments, the NEK7 small molecule inhibitor compound is non-peptidic. In some embodiments, the NEK7 small molecule inhibitor compound is a synthetic compound (i.e., not naturally occurring).

“Hinge-binding element” refers to a region of a NEK7 inhibitor that forms a terminal portion of the molecule that does not contain a direct covalent bond to a hydrophobic back pocket group.

“Flexible linker” refers to a multi-valent (e.g., bivalent, trivalent, etc.) moiety connecting two or more portions of the NEK7 small molecule and comprising one or more rotatable bonds. In some embodiments, the flexible linker is bivalent. In some embodiments, the flexible linker is covalently bound to the hinge-binding element and the urea linker.

“Urea-type linker” refers to multi-valent (e.g., bivalent, trivalent, etc.) moiety connecting two or more portions of the NEK7 small molecule and comprising at least one nitrogen and at least one oxo group as defined herein above. In some embodiments, the urea-type linker is covalently bound to the flexible linker and the hydrophobic back pocket group.

“Hydrophobic back pocket group” refers to a region of a NEK7 inhibitor that forms a terminal portion of the molecule that does not contain a direct covalent bond to a hinge-binding element. In some embodiments, the hydrophobic back pocket group comprises at least one nitrogen.

“Type 1.5 binding mode” refers to a non-covalent interaction of an atom, molecule, or combinations thereof with the ATP binding site of NEK7 that (i) interacts with the hinge region of NEK7 via one or more hydrogen bonds (ii) stabilizes the DLG loop in the “in” confirmation (i.e., proximal from a hinge-binding element of a NEK7 inhibitor) preventing access to the allosteric back pocket space; and (iii) lacks a non-covalent interaction between a compound and the αC helix, maintaining the helix in the “out” catalytically inactive conformation.

“Type 2 binding mode” refers to a non-covalent interaction of an atom, molecule, or combinations thereof with the ATP binding site of NEK7 that (i) interacts with the hinge region of NEK7 via one or more hydrogen bonds; (ii) stabilizes the DLG loop in the “out” confirmation (i.e., distal from a hinge-binding element of a NEK7 inhibitor) allowing access to the allosteric back pocket space; and (iii) forms a non-covalent interaction between the urea-type linker of the NEK7 inhibitor and the αC helix, bringing it in the “in” conformation. In some embodiments, an —NH— group of the hinge-binding element forms one or more hydrogen bonds with ALA114 of NEK7. In some more specific embodiments, two —NH— groups of the urea-type linker forms one or more hydrogen bonds with GLU82 of NEK7. In some embodiments, the type 2 binding mode forces the αC helix to adopt an “in” conformation. In some embodiments, two —NH— groups of the urea-type linker forms one or more hydrogen bonds ASP179 (e.g., via a network of water molecules). In some embodiments, the hydrophobic back pocket group occupies the allosteric back pocket space.

i.e.i.e.e.g. “Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching, but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂-C₈ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching, but does not include carbon atoms belonging to any substituents. Unless stated otherwise specifically in the specification, an alkenyl group is optionally substituted.

The term “alkynyl” refers to unsaturated straight or branched hydrocarbon radical, having 2 to 12 carbon atoms (C₂-C₁₂ alkynyl), two to nine carbon atoms (C₂-C₆ alkynyl), or two to six carbon atoms (C₂-C₆ alkynyl), or any value within these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching, but does not include carbon atoms belonging to any substituents. Unless stated otherwise specifically in the specification, an alkynyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₁₂ alkoxy), or any value within these ranges. Unless stated otherwise specifically in the specification, an alkoxy group is optionally substituted.

“Aminyl” refers to a radical of the formula —NRₐR_{b}, where Rₐ is H or C₁-C₆ alkyl and R_{b} is C₁-C₆ alkyl as defined above. The C₁-C₆ alkyl portion of an aminyl group is optionally substituted unless stated otherwise.

“Aminylalkylcycloalkyl” refers to a radical of the formula —RₐR_{b}NR_{c}R_{d} where Rₐ is cycloalkyl as defined herein, R_{b} is C₁-C₆ alkyl, Rₑ is H or C₁-C₆ alkyl and R_{d} is C₁-C₆ alkyl as defined above. The cycloalkyl and each C₁-C₆ alkyl portion of an aminylalkylcycloalkyl group are optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Cyanoalkyl” refers to an alkyl group comprising at least one cyano substituent. The —CN substituent may be on a primary, secondary or tertiary carbon. Unless stated otherwise specifically in the specification, a cyanoalkyl group is optionally substituted. “Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Alkylcycloalkyl” refers to a radical group of the formula —RₐR_{b} where Rₐ is a cycloalkyl group and R_{b} is an alkyl group as defined above. Unless otherwise stated specifically in the specification, an alkylcycloalkyl group is optionally substituted.

“Fused” refers to any ring structure described herein which is fused to another ring structure.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Haloalkoxy” refers to an alkoxy radical, as defined above, that is substituted by one or more halo radicals, as defined above (e.g., trifluoromethoxy, difluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group is optionally substituted.

“Halocycloalkyl” refers to a cycloalkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a halocycloalkyl group is optionally substituted.

“Haloalkylcycloalkyl” refers to a radical group of the formula —RₐR_{b} where Rₐ is a cycloalkyl group and R_{b} is a haloalkyl group as defined above. Unless otherwise stated specifically in the specification, a haloalkylcycloalkyl group is optionally substituted.

“Halocycloalkylalkyl” refers to a radical group of the formula —RₐR_{b} where Rₐ is an alkyl group and R_{b} is a halocycloalkyl group as defined above. Unless otherwise stated specifically in the specification, a halocycloalkylalkyl group is optionally substituted.

“Heterocyclylcycloalkyl” refers to a radical group of the formula —RₐR_{b} where Rₐ is a cycloalkyl group and R_{b} is a heterocyclyl group as defined herein. Unless otherwise stated specifically in the specification, a heterocyclylcycloalkyl group is optionally substituted.

“Hydroxylalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxylalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen and sulfur. Unless stated otherwise specifically in the specification, the heterocyclyl radical is partially or fully saturated and is a monocyclic, bicyclic, tricyclic or tetracyclic ring system, which may include fused, spirocyclic and/or bridged ring systems. Nitrogen, carbon and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“Haloheterocyclyl” refers to a heterocyclyl group comprising at least one halo substituent. The halo substituent may be on a primary, secondary or tertiary carbon. Unless stated otherwise specifically in the specification, a haloheterocyclyl group is optionally substituted.

“Haloheterocyclylalkyl” refers to a radical group of the formula —RₐR_{b} where Rₐ is an alkyl group and R_{b} is a haloheterocyclyl group as defined herein. Unless otherwise stated specifically in the specification, a haloheterocyclylalkyl group is optionally substituted.

“Heterocyclylalkyl” refers to a radical group of the formula —RₐR_{b} where Rₐ is an alkyl group and R_{b} is a heterocyclyl group as defined herein. Unless otherwise stated specifically in the specification, a heterocyclylalkyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, t-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

Oxazolyl, isoxazolyl, 1, 2, 3-oxadiazolyl, 1, 2,4-oxadiazolyl, 1,2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl refer to the following structures, respectively:

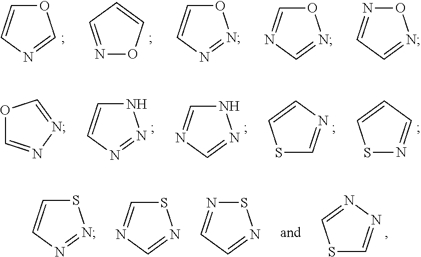

wherein the oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl, and 1, 3, 4-thiadiazolyl are attached to the remainder of the molecule by a covalent bond to one of the carbon atoms in the ring of the oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl, and 1, 3, 4-thiadiazolyl.

The term “substituted” as used herein means any of the above groups (e.g., alkyl, alkenyl, alkylene, alkylcarbonyl, alkoxy, alkoxyalkyl, aminylalkyl, aryl, cyanoalkyl, cycloalkyl, haloalkyl, heterocyclyl, heterocyclene, heterocyclylalkyl, heteroaryl, heteroarylalkyl and/or hydroxylalkyl) wherein at least one hydrogen atom (e.g., 1, 2, 3 or all hydrogen atoms) is replaced by a bond to a non-hydrogen substituent. Examples of non-hydrogen substituents include, but are not limited to, amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl and/or hydroxylalkyl substituents, each of which may also be optionally substituted with one or more of the above substituents.

In some specific embodiments, the optional substitutions are independently selected from the group consisting of halo, hydroxyl, cyano, aminyl, C₁-C₆ alkyl, C₂-C₆ alkenyl, C₂-C₆ alkynyl, C₁-C₆ haloalkyl, C₃-C₈ cycloalkyl, C₃-C₈ halocycloalkyl, C₆-C₁₀ aryl, 5- or 6-membered heteroaryl, C₁-C₆ alkoxy and 3-8 membered heterocyclyl.

The term “effective amount” or “therapeutically effective amount” refers to that amount of a compound described herein that is sufficient to effect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S M. Berge, et al., “Pharmaceutical Salts”, J. Pharm. Sci., 1977, 66:1-19, and Handbook of Pharmaceutical Salts., Properties, Selection, and Use, Stahl and. Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66.1-19, and Handbook of Pharmaceutical Salts, Properties, Selection, and Use, Stahl and Wermuth, Eds., Wiley-VCH and VHCA, Zurich, 2002 Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

The terms “antagonist” and “inhibitor” are used interchangeably, and they refer to a compound having the ability to inhibit a biological function of a target protein, whether by inhibiting the activity or expression of the protein, such as NLRP3 inflammasome or NEK7 or the association of NLRP3 inflammasome—NEK7. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definitio.

“Signal transduction” is a process during which stimulatory or inhibitory signals are transmitted into and within a cell to elicit an intracellular response.

The term “selective inhibition” or “selectively inhibit” refers to a biologically active agent refers to the agent's ability to preferentially reduce the target signaling activity as compared to off-target signaling activity, via direct or indirect interaction with the target.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi. T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of the disclosed compounds Structure (I) (e.g., compound of Structure (Ia), (Ib), (Ic), (Id), (Ie), and (If))

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some aspects, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (e.g., compounds of Structure (I), (Ia), (Ib), (Ic), (Id), (Ie), and (If)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)-for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I), (Ia), (Ib), (Ic), (Id), (Ie), or (If) are a mixture of enantiomers or diastereomers. In other embodiments, the compounds of Structure (I), (Ia), (Ib), (Ic), (Id), (Ie), or (If) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency

### Compositions and Compounds

In embodiments, the disclosure provides compositions comprising compounds including pharmaceutically acceptable salts, stereoisomers and prodrugs thereof, which are capable of modulating the activity of NEK7 and/or the NLRP3 inflammasome. Some embodiments provide methods of administering the NEK7 small molecule inhibitor compounds while other embodiments provide compositions comprising the NEK7 small molecule inhibitor compounds and at least one NEK7 protein. Accordingly, one embodiment provides a composition comprising at least one NEK7 protein and a NEK7 small molecule inhibitor compound comprising at least one of the following features:

i. a hinge-binding element comprising at least one hydrogen donor and at least one hydrogen acceptor;

ii a flexible linker;

iii. a urea-type linker; or

iv. a hydrophobic back pocket group.

In some embodiments, hinge-binding element comprises a hydrogen attached to a nitrogen as a hydrogen donor. In certain embodiments, the hinge-binding element comprises a nitrogen with a lone electron pair as a hydrogen acceptor in some more specific embodiments, the hinge-binding element comprises a heteroaryl. In more specific embodiments, the hinge-binding element comprises a bicyclic heteroaryl. In more specific embodiments, the hinge-binding element comprises a fused bicyclic heteroaryl. In certain embodiments, the hinge-binding element has the following structure:

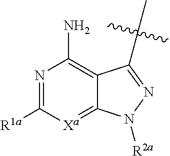

wherein:

X^{a} is N or CH;

R^{1a} is H or C₁-C₆ alkyl; and

R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl.

In more specific embodiments, X^{a} is N. In some embodiments, R^{1a} is H or methyl. In certain embodiments, R^{2a} has one of the following structures:

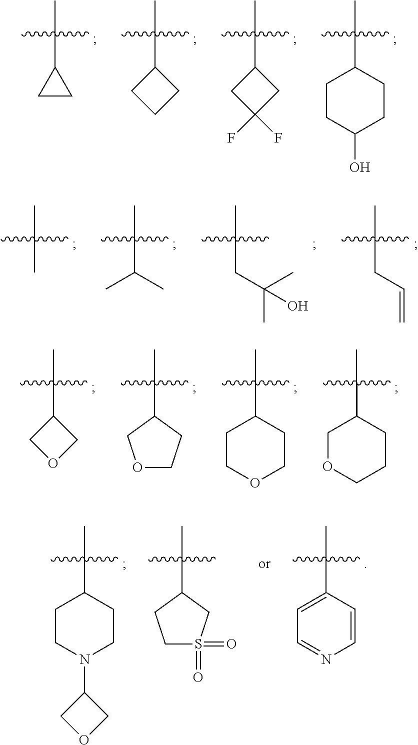

In certain embodiments, the hinge-binding element has the following structure:

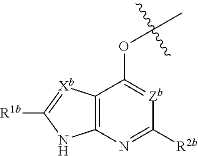

wherein:

X^{b} is N or CR^{10b};

Z^{b} is N or CR^{11b}; and

R^{1b}, R^{2b}, R^{10b}, and R^{1b} are each independently H, halo, C₁-C₆ alkyl, or C₃-C₈ cycloalkyl.

In certain embodiments, X^{b} is N or CH. In some embodiments, X^{b} is CR^{10b} and R^{10b} is chloro, methyl, or cyclopropyl. In certain embodiments, Z^{b} is N or CH. In more specific embodiments, Z^{b} is CR^{11b} and R^{11b} is halo, C₁-C₆ alkyl, or C₃-C₈ cycloalkyl.

In some embodiments, R^{1b} is H or methyl. In some embodiments, R^{2b} is H. In some embodiments, the hinge-binding element has the following structure:

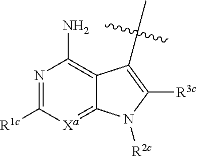

wherein:

X^{c} is CH or N;

R^{1c} is H or C₁-C₆ alkyl;

R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl; and

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl and C₁-C₆ alkoxy.

In some embodiments, X^{c} is N. In certain embodiments, X^{c} is CH. In some more specific embodiments, R^{1c} is H or methyl.

In some embodiments, R^{2c} has one of the following structures:

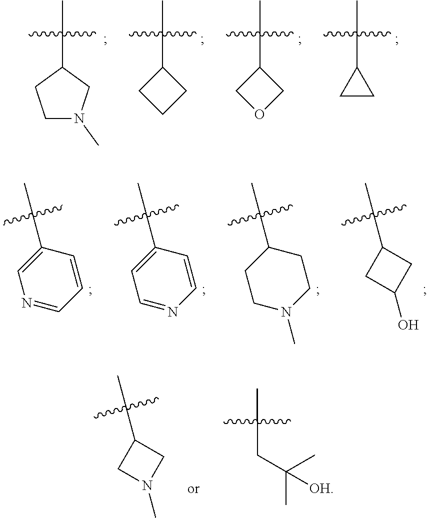

In certain embodiments, R^{3c} is H or methyl. In some embodiments, the hinge-binding element has the following structure:

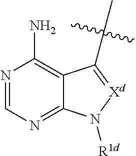

wherein:

X^{d} is N or CR^{4d};

R^{1d} is C₁-C₆ alkyl, C₁-C₆ hydroxylalkyl, C₃-C₁₀ cycloalkyl, or 3-10 membered heterocyclyl, and

R^{4d} is H, C₁-C₆ alkyl, C₁-C₆ haloalkyl, or C₃-C₈ cycloalkyl.

In some embodiments, X^{d} is N. In more specific embodiments, X^{d} is CH. In some embodiments, R^{1d} has one of the following structures:

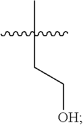 [SMILES: *CCO]

### —CH₃;

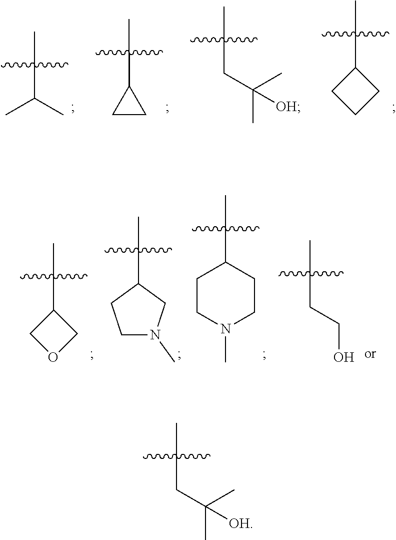

In some embodiments, the hinge-binding element has the following structure:

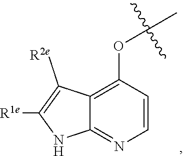

wherein:

R^{1e} is H, halo, C₁-C₆ alkyl, C₃-C₁₀ cycloalkyl, or 3-10 membered heterocyclyl; and

R^{2e} is H, halo, C₁-C₆ alkyl, C₁-C₆ haloalkyl, C₃-C₁₀ cycloalkyl, or 3-10 membered heterocyclyl.

In certain embodiments, R^{1e} is H, methyl, fluoro, or chloro. In more specific embodiments, R^{2e} is chloro, fluoro, cyclopropyl, or methyl.

In some embodiments, the flexible linker comprises at least one cycloalkyl, heterocyclyl, aryl, or heteroaryl. In some embodiments, the flexible linker is monocyclic or bicyclic. In more embodiments, the flexible linker is a fused bicyclic. In certain embodiments, the flexible linker has one of the following structures:

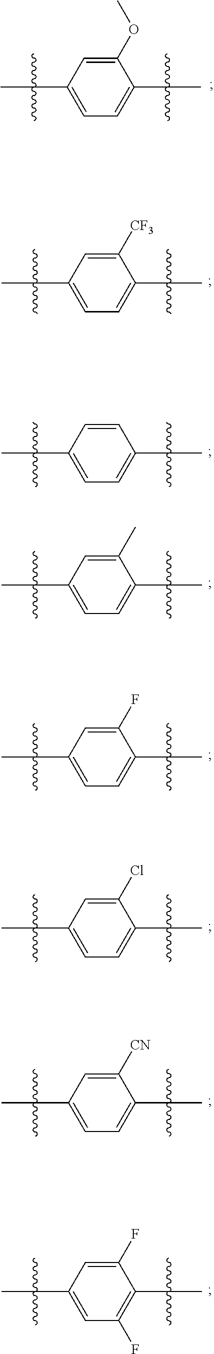

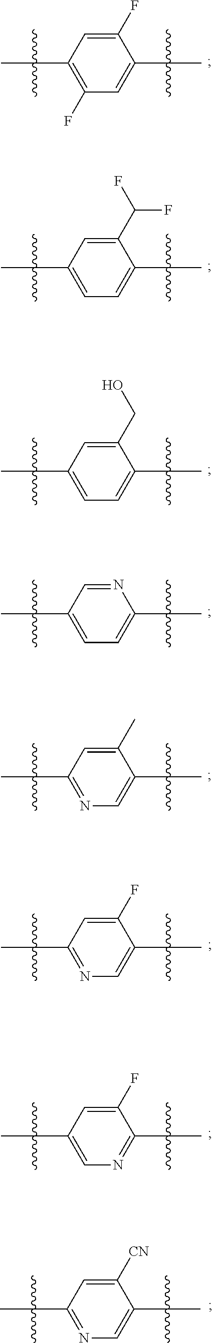

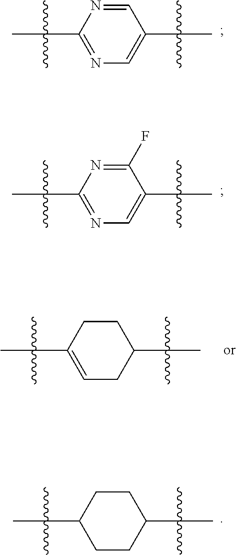 [SMILES: *c1cnc(*)nc1 ; *c1ncc(*)c(F)n1 ; *C1=CCC(*)CC1 ; *C1CCC(*)CC1]

In some embodiments, the urea-type linker comprises the following structure:

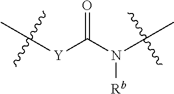

wherein:

Y is C(R^{c})(R^{d}), or NR^{b};

R^{a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl;

R^{b} is H, C₁-C₆ alkyl, C₂-C₆ alkenyl, C₂-C₆ alkynyl, C₃-C₈ cycloalkyl, or C₁-C₆ hydroxylalkyl;

R^{c} is H, C₁-C₆ alkyl, C₂-C₆ alkenyl, C₂-C₆ alkynyl, or C₁-C₆ hydroxylalkyl; and

R^{d} is H, OH, C₁-C₆ alkyl, C₂-C₆ alkenyl, C₂-C₆ alkynyl, or C₁-C₆ hydroxylalkyl.

In some embodiments, the urea-type linker has one of the following structures:

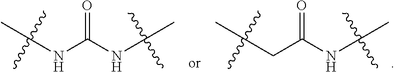 [SMILES: *NC(=O)N* ; *CC(=O)N*]

In some embodiments, the hydrophobic back pocket group comprises an aryl or heteroaryl. In certain embodiments, the hydrophobic back pocket group has one of the following structures:

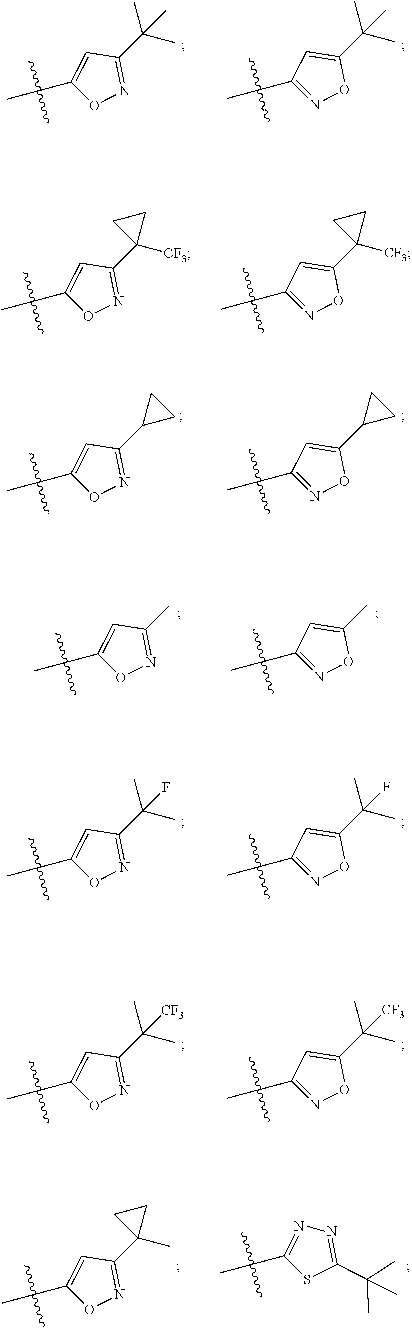

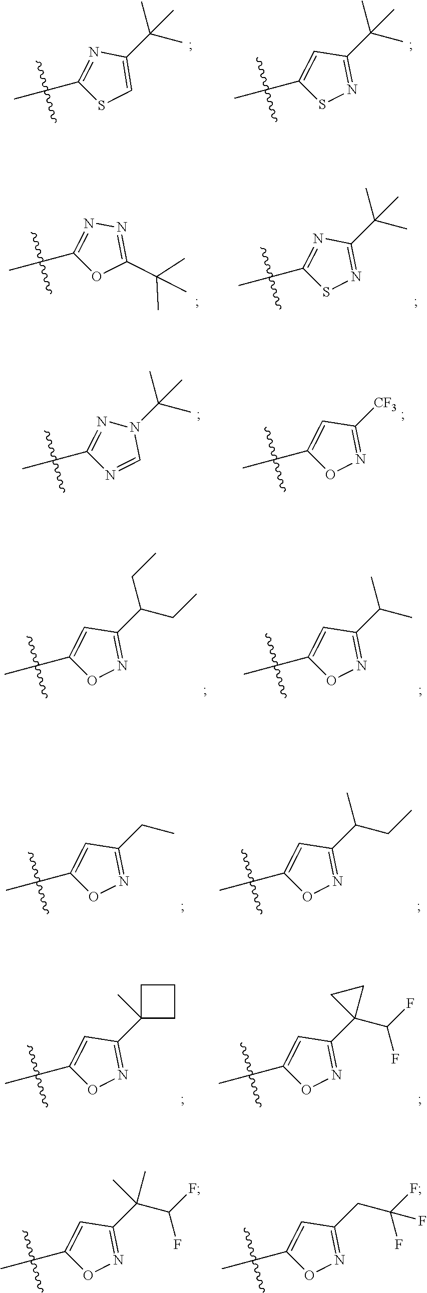

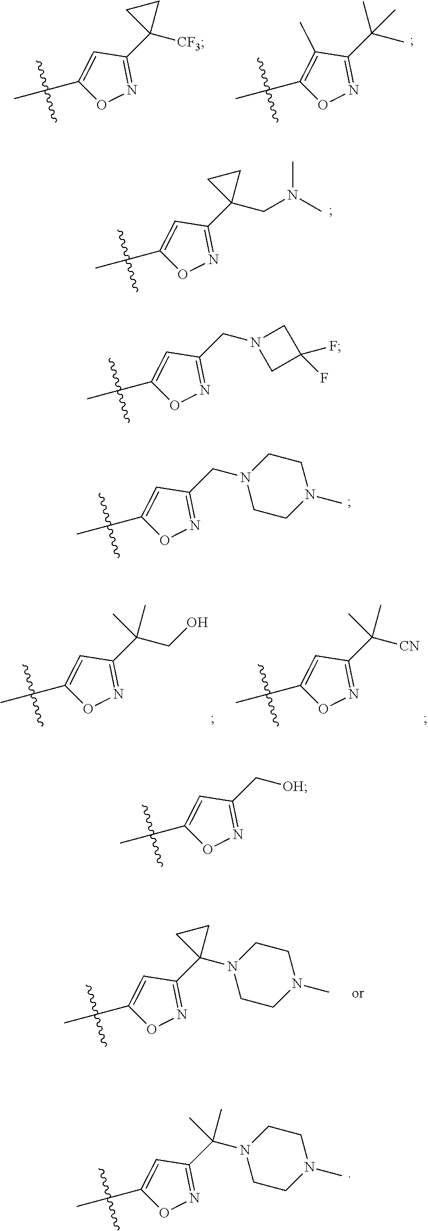

In some embodiments, hydrophobic back pocket group has the following structure:

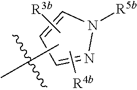

wherein:

R^{3b} and R^{4b} are each independently H, halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl, provided that R^{2a} and R^{2b} are not both H; and

R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy.

In certain embodiments, R^{3b} is tert-butyl, methyl, or cyclopropyl. In certain embodiments. R^{3b} has the following structure:

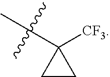 [SMILES: *C1(C(F)(F)F)CC1]

In some embodiments, R^{4b} is H. In certain embodiments, R^{5b} has one of the following structures:

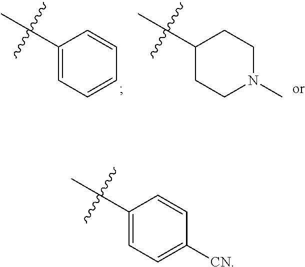

In some embodiments, the hydrophobic back pocket group has one of the following structures:

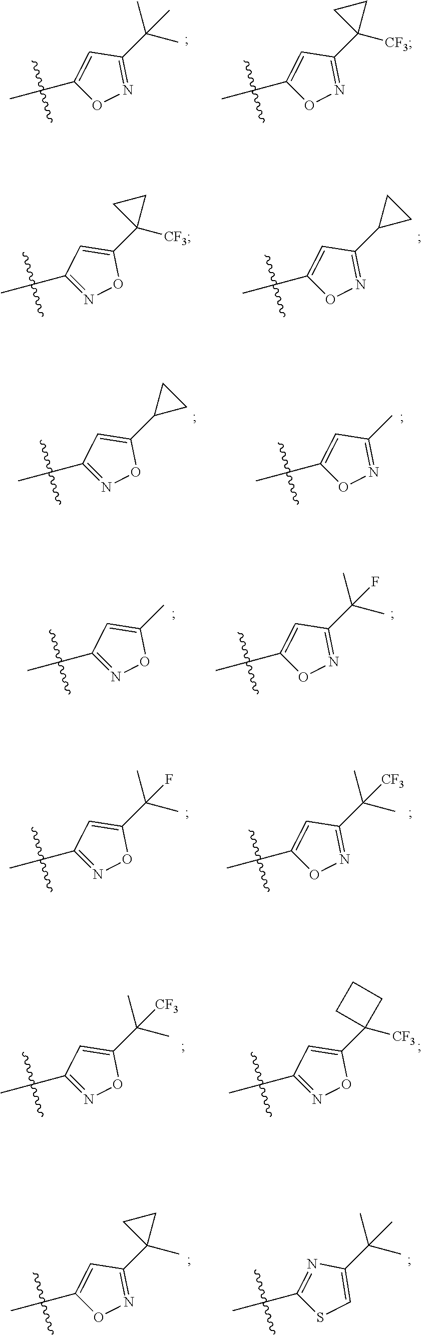

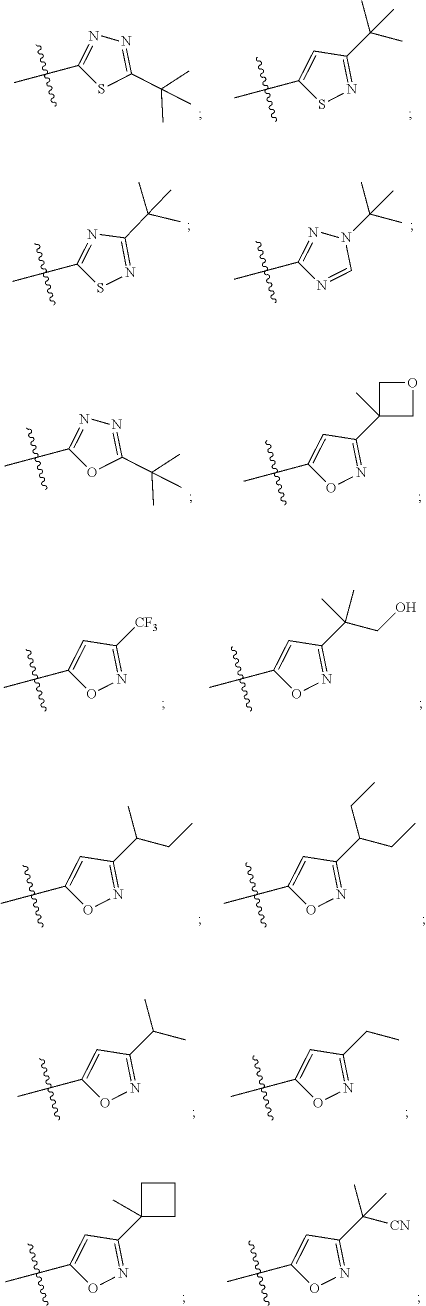

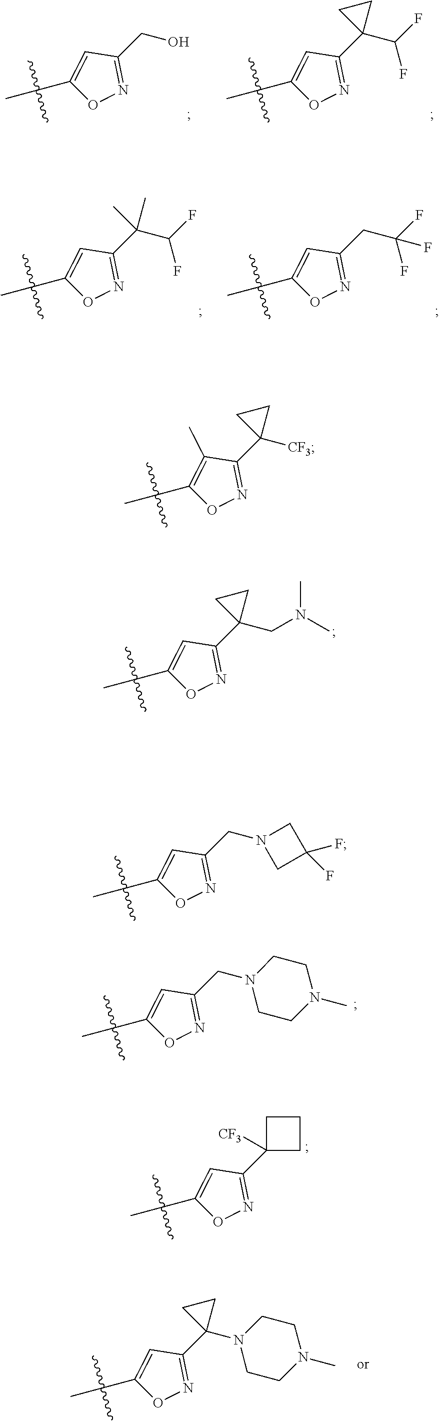

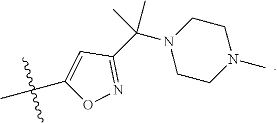 [SMILES: *c1cc(C(C)(C)N2CCN(C)CC2)no1]

In some embodiments, the hydrophobic back pocket group has the following structure:

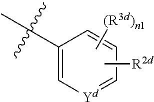

wherein:

Y^{d} is N or CH;

R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;

R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl; and

n1 is 0, 1, 2, 3, or 4.

In some embodiments, Y^{d} is CH. In certain embodiments, Y^{d} is N.

In certain embodiments, R^{2d} has one of the following structures

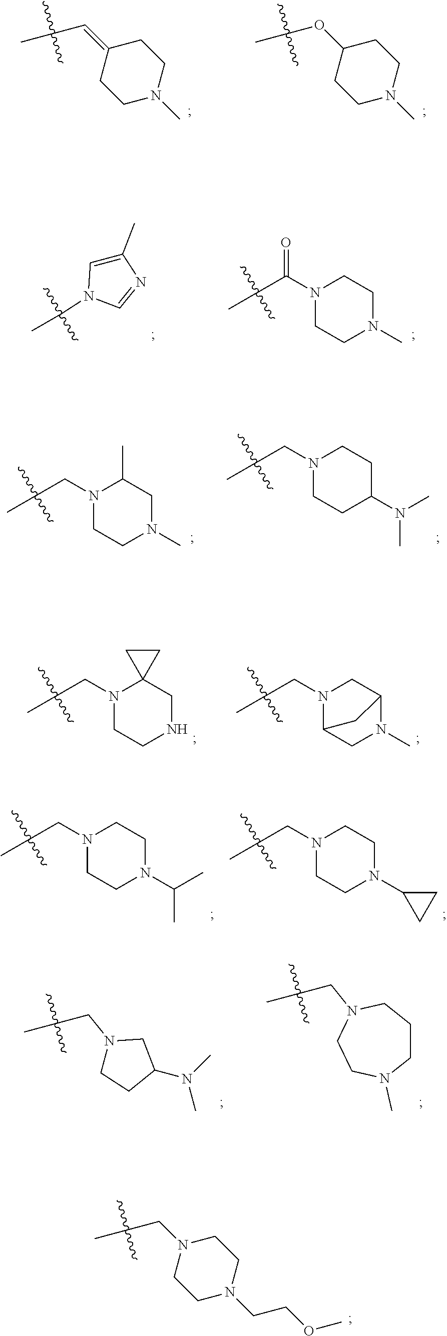

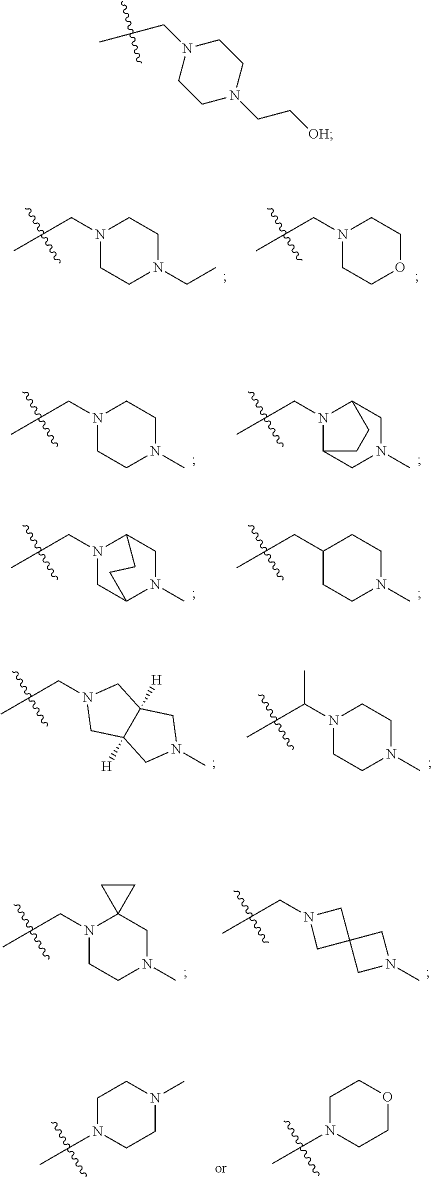

In some specific embodiments, n1 is 0. In certain embodiments, n1 is 1 or 2 and each R^{3d} is independently fluoro, chloro, trifluoromethyl, difluoromethyl, trifluoromethoxy, methyl, methoxy, cyclopropyl, or cyano.

In more specific embodiments, the hydrophobic back pocket group has the following structure:

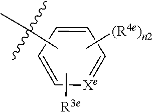

wherein:

X^{c} is N or CH;

R^{3c} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;

R^{4c} is, at each occurrence, independently halo, cyano, C₁-C₆, alkyl, C₁-C₆ haloalkyl, C₁-C₆ alkoxy, C₁-C₆ haloalkoxy, C₃-C₈ halocycloalkyl, or C₃-C₈ cycloalkyl; and

n2 is 0, 1, 2, 3, or 4.

In some embodiments, X^{e} is CH. In certain embodiments, X^{e} is N. In some embodiments, R^{3e} has one of the following structures:

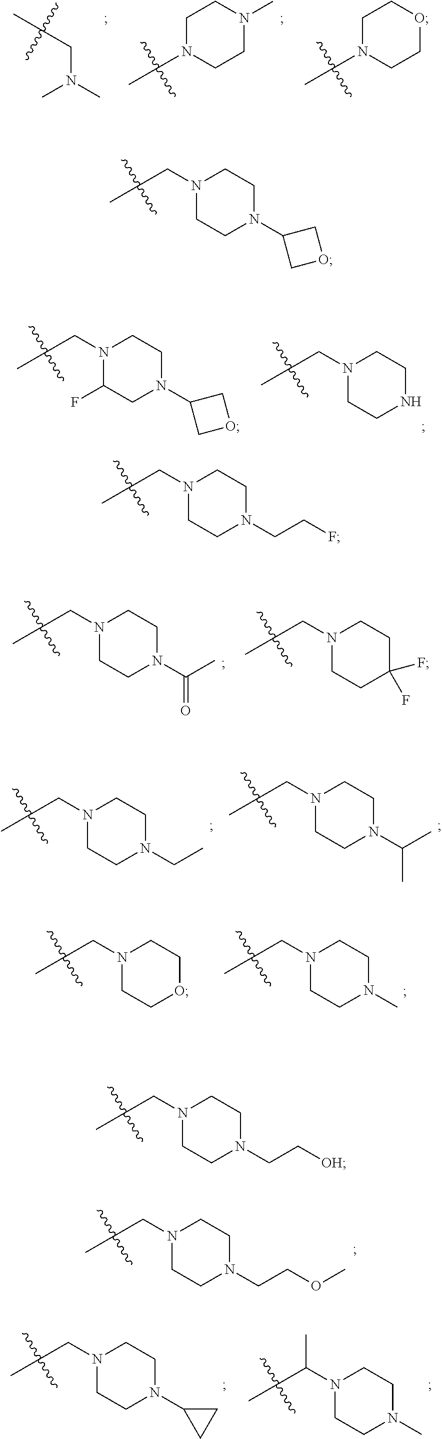

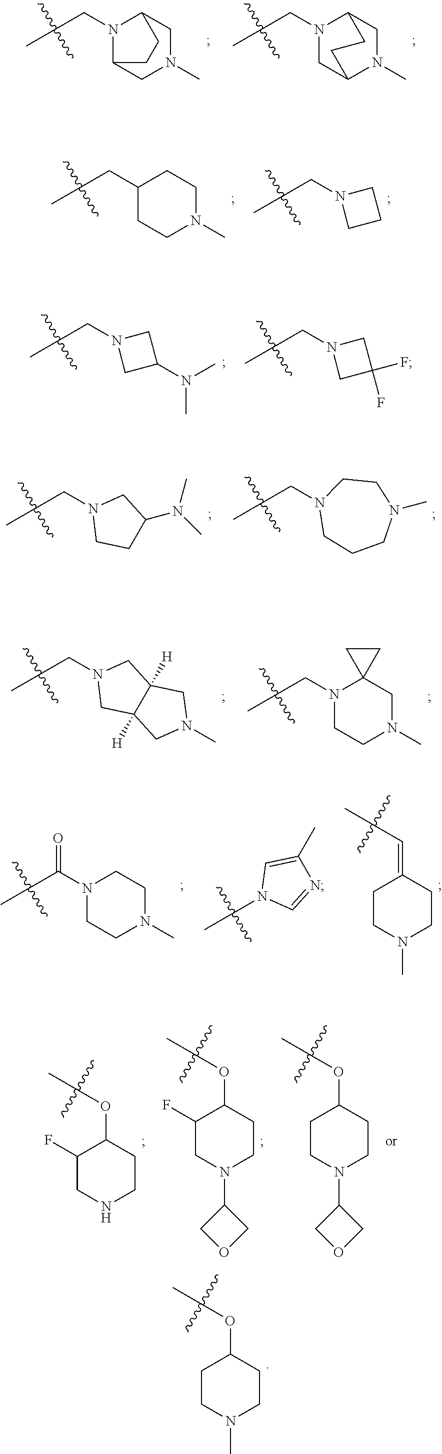

In some embodiments, n2 is 1 and R^{4c} is trifluoromethyl, difluoromethyl, fluoro, chloro, methyl, cyclopropyl, methoxy, or 2,2-difluorocyclopropyl.

In certain embodiments, the NEK7 small molecule inhibitor compound comprises two or more of the following features:
i. a hinge-binding element comprising at least one hydrogen donor and at least one hydrogen acceptor;
ii. a flexible linker;
iii. a urea-type linker, or
iv. a hydrophobic back pocket group.

In some embodiments, the NEK7 small molecule inhibitor compound comprises three or more of the following features:
i. a hinge-binding element comprising at least one hydrogen donor and at least one hydrogen acceptor;
ii. a flexible linker;
iii. a urea-type linker; or
iv. a hydrophobic back pocket group.

In more specific embodiments, the NEK7 small molecule inhibitor compound comprises each of the following features:
i. a hinge-binding element comprising at least one hydrogen donor and at least one hydrogen acceptor.
ii. a flexible linker;
iii. a urea-type linker; or
iv. a hydrophobic back pocket group.

In some embodiments, the NEK7 small molecule inhibitor compound is non-peptidic. In some embodiments, the NEK7 small molecule inhibitor compound is synthetic.

In some embodiments, the NEK7 small molecule inhibitor compound has the following Structure (I):

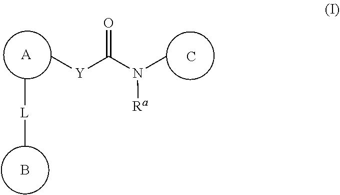

wherein:

A is cycloalkyl, heterocyclyl, aryl, or heteroaryl:

B is a heteroaryl ring;

C is aryl or heteroaryl;

L is a direct bond or —O—:

Y is C(R^{c})(R^{d}), or NR^{b};

R^{a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl;

R^{b} is H, C₁-C₆ alkyl, C₂-C₆ alkenyl, C₂-C₆ alkynyl, C₃-C₈ cycloalkyl, or C₁-C₆ hydroxylalkyl;

R^{c} is H, C₁-C₆ alkyl, C₂-C₆ alkenyl, C₂-C₆ alkynyl, or C₁-C₆ hydroxylalkyl; and

R^{d} is H, OH, C₁-C₆ alkyl, C₂-C₆ alkenyl, C₂-C₆ alkynyl, or C₁-C₆ hydroxylalkyl.

In some embodiments, A has one of the following structures:

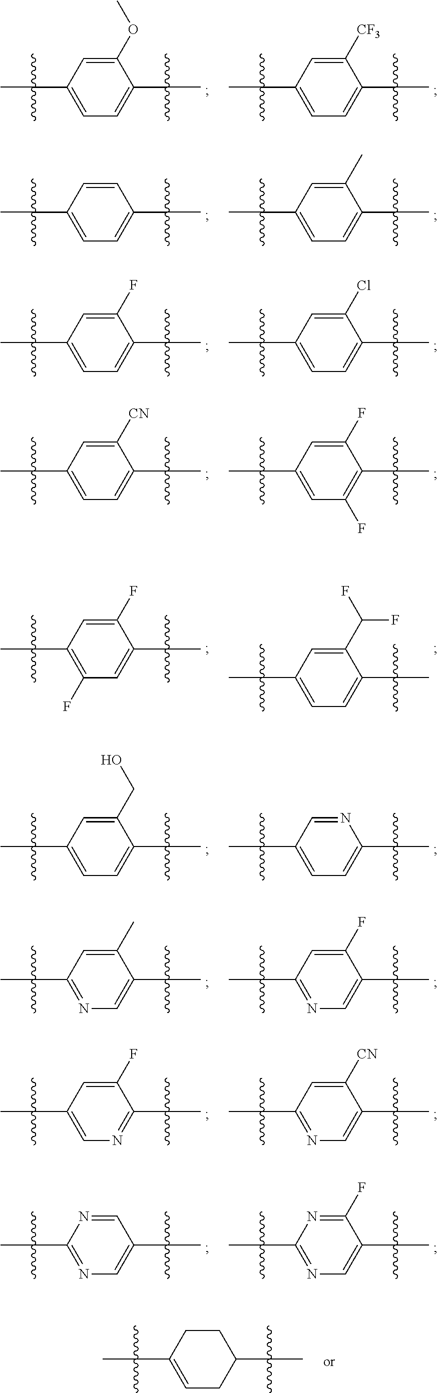

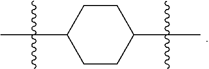 [SMILES: *C1CCC(*)CC1]

In some embodiments, B is has the following structure:

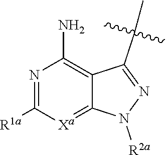

wherein:

X^{a} is N or CH;

R^{1a} is H or C₁-C₆ alkyl; and

R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl.

In some embodiments, X^{a} is N. In certain embodiments. R^{1a} is H or methyl. In some embodiments, R^{2a} has one of the following structures:

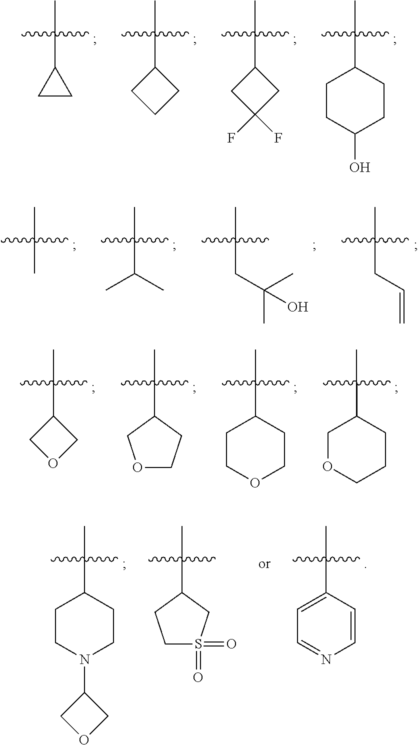

In some embodiments, B has the following structure:

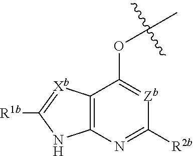

wherein:

X^{b} is N or CR^{10b};

Z^{b} is N or CR^{11b}; and

R^{1b}, R^{2b}, R^{10b}, and R^{11b} are each independently H, halo, C₁-C₆ alkyl, or C₃-C₈ cycloalkyl.x. The method of claim x, wherein X^{b} is N or CH.

In some embodiments, X^{b} is CR^{10b} and R^{11b} is chloro, methyl, or cyclopropyl. In certain embodiments, Z^{b} is N or CH. In certain embodiments, Z^{b} is CR^{11b} and R^{11b} is halo, C₁-C₆ alkyl, or C₃-C₈ cycloalkyl. In more specific embodiments, R^{1b} is H or methyl. In some embodiments, R^{2b} is H.

In certain embodiments, B has the following structure:

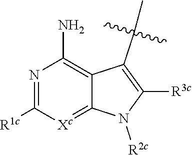

wherein:

X^{e} is CH or N;

R^{1c} is H or C₁-C₆ alkyl;

R^{2c} is C₁-C₆ alkyl, C₁-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl; and

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl and C₁-C₆ alkoxy.

In some embodiments, X^{c} is N. In more specific embodiments, X^{c} is CH. In some embodiments, R^{1c} is H or methyl. In certain embodiments, R^{2c} has one of the following structures:

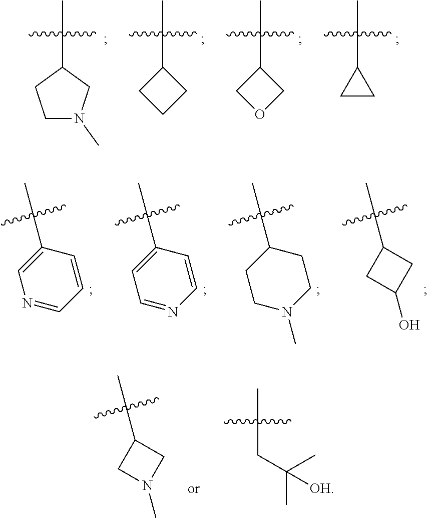

In certain embodiments, R^{3c} is H or methyl. In some embodiments, B has the following structure:

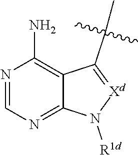

wherein:

X^{d} is N or CR^{4d};

R^{1d} is C₁-C₆ alkyl, C₁-C₆ hydroxylalkyl, C₃-C₁₀ cycloalkyl, or 3-10 membered heterocyclyl; and

R^{4d} is H, C₁-C₆ alkyl, C₁-C₆ haloalkyl, or C₃-C₈ cycloalkyl.

In certain embodiments, X^{d} is N. In some embodiments, X^{d} is CH. In some embodiments, R^{1d} has one of the following structures:

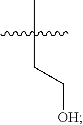 [SMILES: *CCO]

### —CH₃;

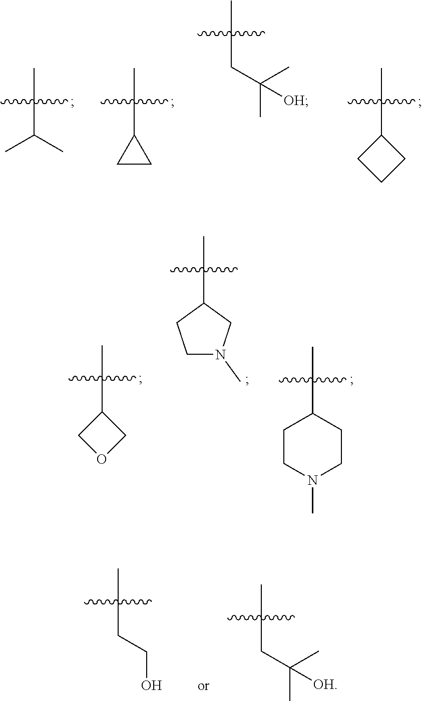

In certain embodiments, B has the following structure:

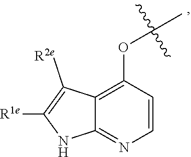

wherein:

R^{1e} is H, halo, C₁-C₆ alkyl, C₃-C₁₀ cycloalkyl, or 3-10 membered heterocyclyl, and

R^{2e} is H, halo, C₁-C₆ alkyl, C₁-C₆ haloalkyl, C₃-C₁₀ cycloalkyl, or 3-10 membered heterocyclyl.

In some embodiments, R^{1e} is H, methyl, fluoro, or chloro. In some embodiments, R^{2e} is chloro, fluoro, cyclopropyl, or methyl.

In certain embodiments, C has one of the following structures:

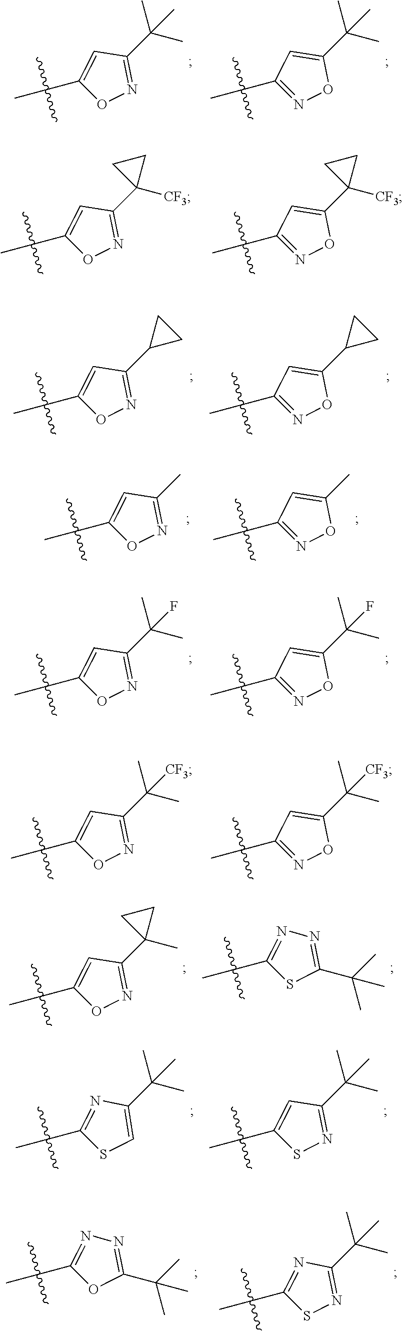

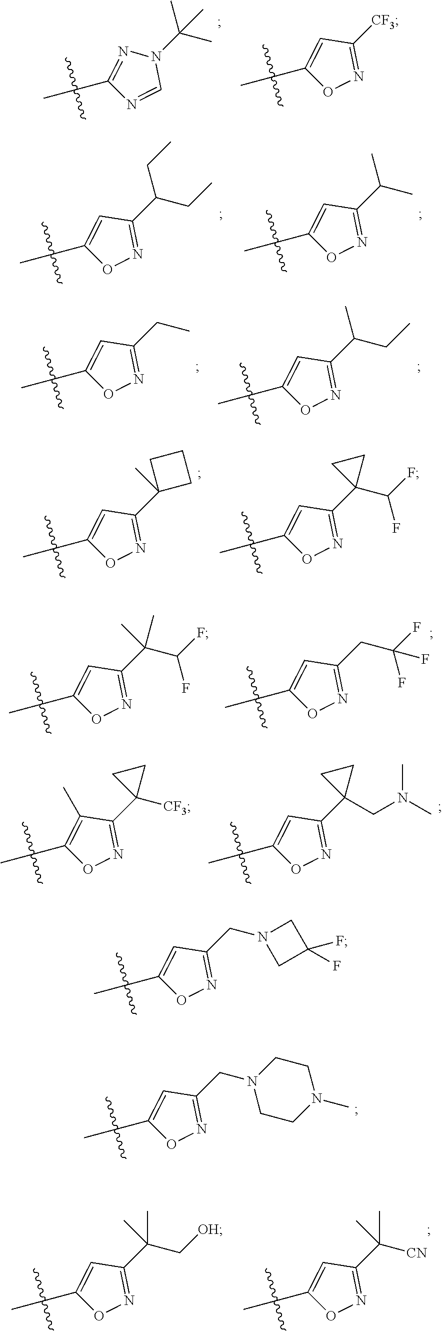

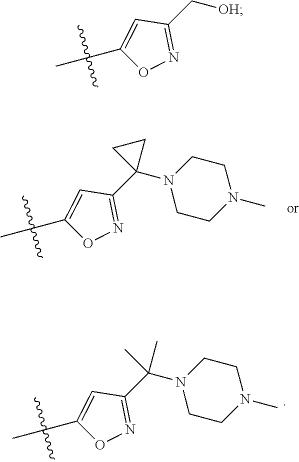 [SMILES: *c1cc(CO)no1 ; *c1cc(C2(N3CCN(C)CC3)CC2)no1 ; *c1cc(C(C)(C)N2CCN(C)CC2)no1]

In some embodiments, C has the following structure:

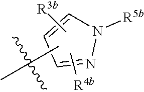

wherein:

R^{3b} and R^{4b} are each independently H, halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl, provided that R^{2a} and R^{2b} are not both H; and

R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy.

In some embodiments, R^{3b} is tert-butyl, methyl, or cyclopropyl. In some more specific embodiments, R^{3b} has the following structure:

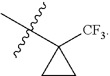 [SMILES: *C1(C(F)(F)F)CC1]

In some embodiments, R^{4b} is 1. In more specific embodiments, R^{5b} has one of the following structures:

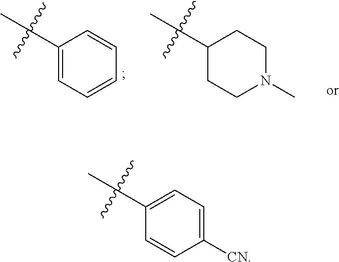

In some embodiments, C has one of the following structures:

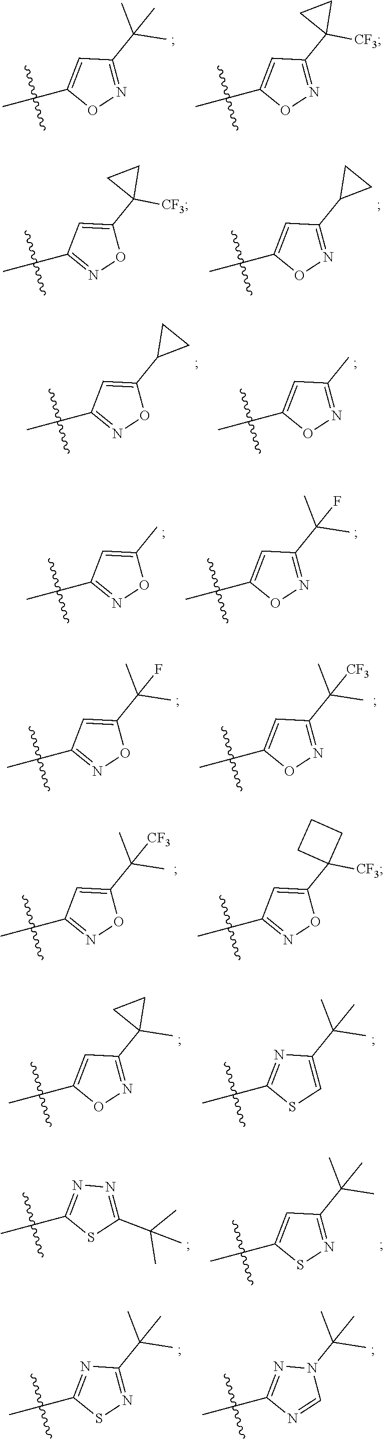

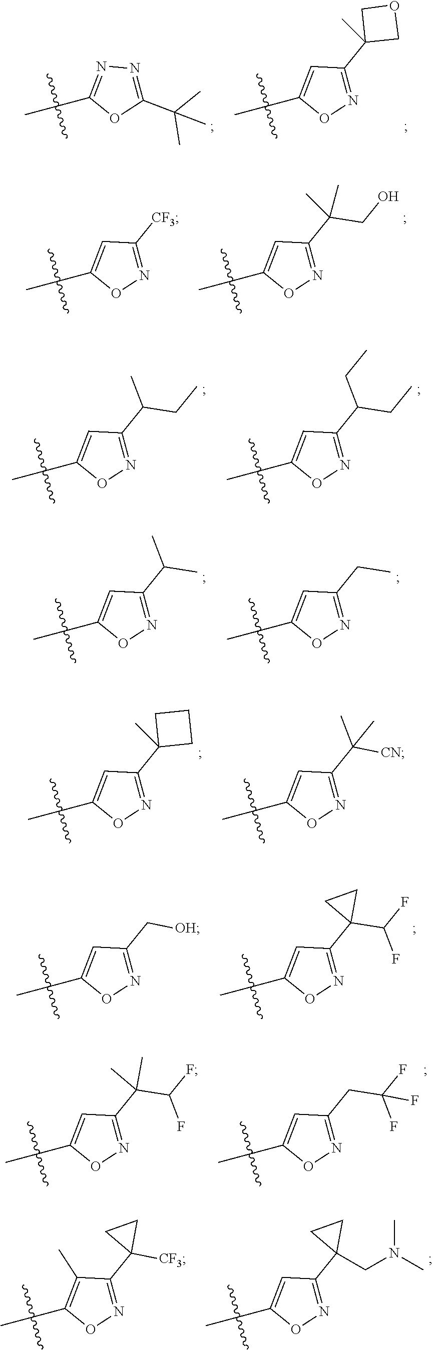

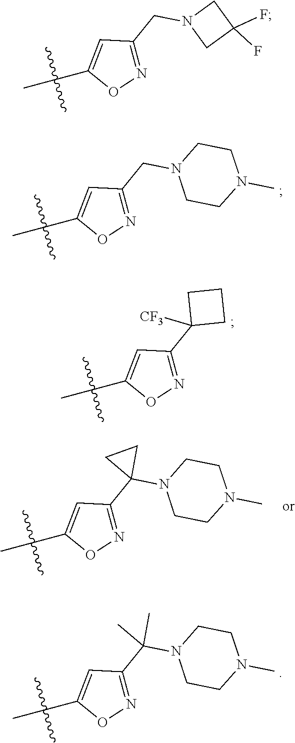

In certain embodiments, C has the following structure:

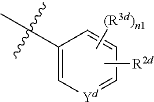

wherein:

Y^{d} is N or CH;

R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;

R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl; and

n1 is 0, 1, 2, 3, or 4.

In some embodiments, Y^{d} is CH. In more specific embodiments, Y^{d} is N.

In some specific embodiments, R^{2d} has one of the following structures:

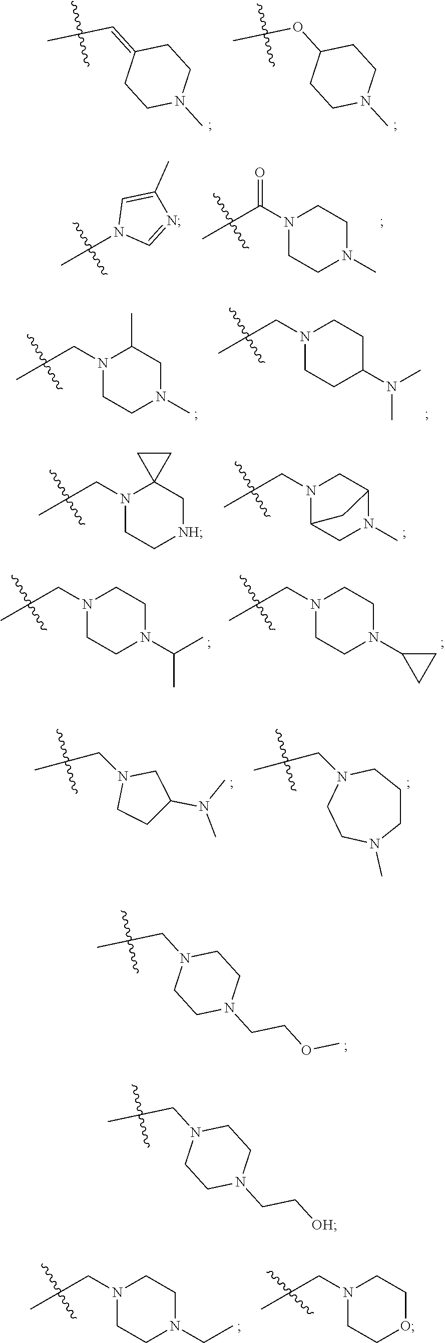

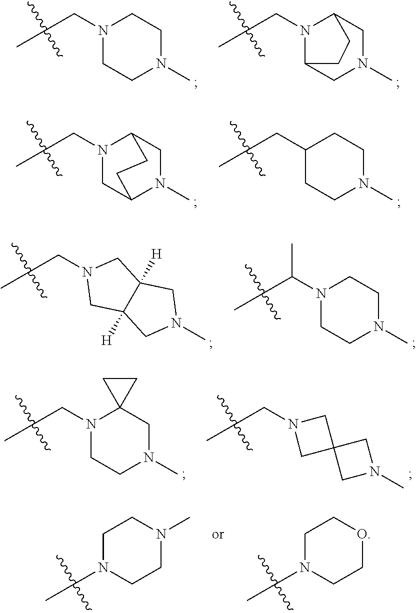

In certain embodiments, n1 is 0. In some embodiments, n1 is 1 or 2 and each R^{3d} is independently fluoro, chloro, trifluoromethyl, difluoromethyl, trifluoromethoxy, methyl, methoxy, cyclopropyl, or cyano. In some embodiments, C has the following structure:

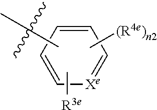

wherein:

X^{e} is N or CH;

R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl:

R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and

n2 is 0, 1, 2, 3, or 4.

In certain embodiments, X^{e} is CH. In some embodiments, X^{e} is N. In certain embodiments, R^{3e} has one of the following structures:

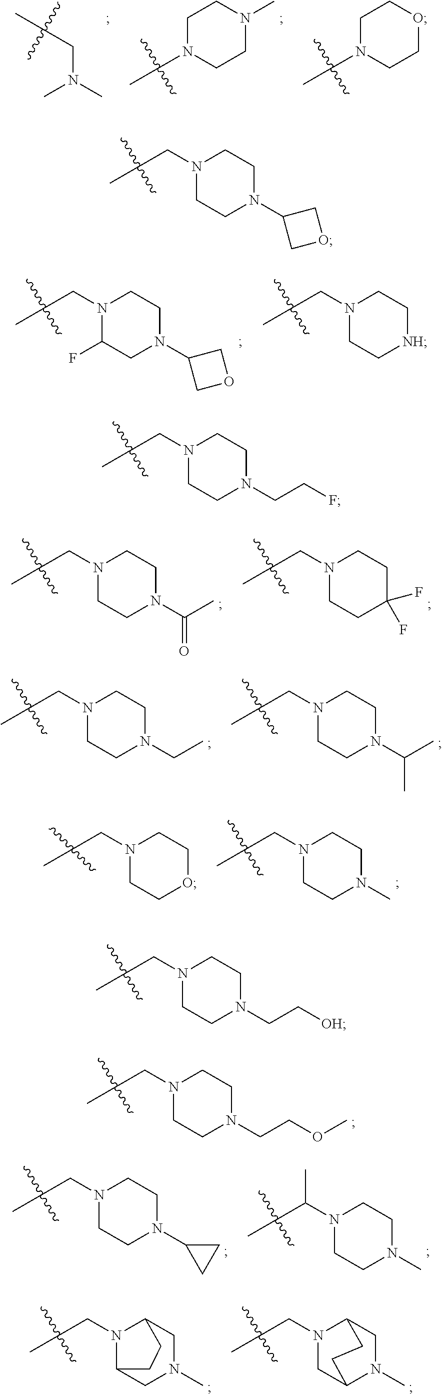

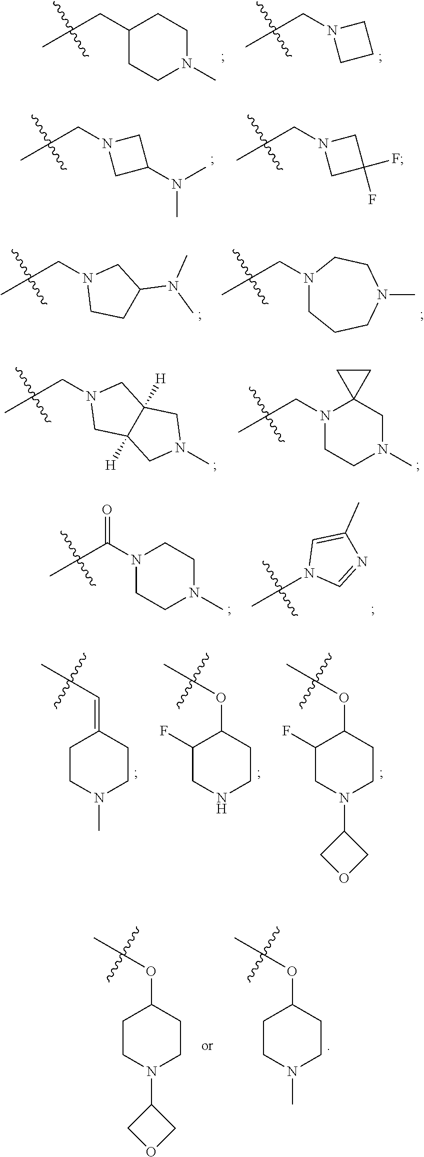

In certain embodiments, n2 is 1 and R^{4e} is trifluoromethyl, difluoromethyl, fluoro, chloro, methyl, cyclopropyl, methoxy, or 2,2-difluorocyclopropyl.

In some embodiments, the NEK7 small molecule inhibitor compound has the following Structure (Ia):

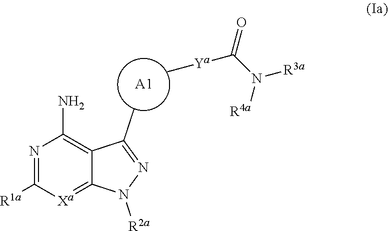

or a pharmaceutically acceptable salt, stereoisomer or prodrug thereof, wherein:

A1 is C₆-C₁₀ aryl, C₃-C₁₀ cycloalkyl, 3-10 membered heterocyclyl or 5-6 membered monocyclic heteroaryl, each of which is optionally substituted with one or more R⁵;

X^{a} is N or CH;

Y^{a} is CHOH or NH;

R^{1a} is H or C₁-C₆ alkyl;

R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₁-C₆ alkynyl, C₁-C₆ alkoxy and 3-8 membered heterocyclyl;

R^{3a} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from amino, halo, cyano, C₁-C₆, alkyl, C₂-C₆ alkenyl, C₂-C₆ alkynyl, C₁-C₆ haloalkyl, C₃-C₈ cycloalkyl, C₃-C₈ alkylcycloalkyl, C₃-C₈ haloalkylcycloalkyl, C₃-C₈ aminylalkylcycloalkyl, C₁-C₆ cyanoalkyl, C₁-C₆ aminyl, C₁-C₆ hydroxylalkyl, 3-8 membered heterocyclyl, 3-8 membered heterocyclylalkyl, 3-8 membered heterocyclylcycloalkyl, 3-8 membered haloheterocyclyl, 3-8 membered haloheterocyclylalkyl, C₃-C₈ halocycloalkyl and C₃-C₈ halocycloalkylalkyl, and combinations thereof;

R^{4a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl or 5- or 6-membered heteroaryl, each of which is optionally substituted with one more substituents selected from halo, hydroxyl, cyano, aminyl. C₁-C₆ alkyl, C₂-C₆ alkenyl, C₂-C₆ alkynyl and C₁-C₆ alkoxy; and

R^{5a} is, at each occurrence, independently halo, cyano, C₁-C₆ alkyl, C₁-C₆ hydroxylalkyl or C₁-C₆ haloalkyl.

In certain embodiment, R^{1a} is H. In other embodiments, R^{1a} is C₁-C₆ alkyl, such as methyl.

In one embodiment, compounds of Structure (Ia) are provided, where R^{2a} is branched C₄-C₆ alkyl, C₃-C₄ cycloalkyl, C₃-C₈ heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In another embodiment, compounds of Structure (Ia) are provided, where Rₐ is branched C₄-C₆ alkyl, C₃-C₄ cycloalkyl, or C₃-C₈ heterocyclyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In specific embodiments, R^{2a} is cyclopropyl, cyclobutyl, cyclopentyl or cyclohexyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In different embodiments, R^{2a} is methyl, isopropyl, 2-methylpropyl or allyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In other embodiments, R^{2a} is oxetanyl, tetrahydrofuranyl, tetrahydropyranyl, piperidinyl or dioxidotetrahydrothiophenyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In other embodiments, R^{2a} is oxetanyl, tetrahydrofuranyl, tetrahydropyranyl, piperidinyl, azetidinyl or dioxidotetrahydrothiophenyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy, and 3- to 8-membered heterocyclyl.

In still more embodiments, R^{2a} is pyridinyl optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In any of the foregoing embodiments, R^{2a} is unsubstituted. In other of the foregoing embodiments, R^{2a} is substituted with one or more of hydroxyl and fluoro.

In more specific embodiments, R^{2a} has one of the following structures:

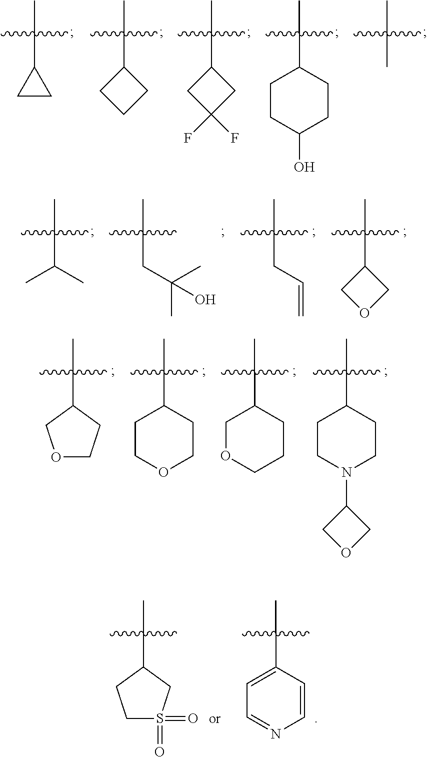

In further specific embodiments, R^{2a} has one of the following structures:

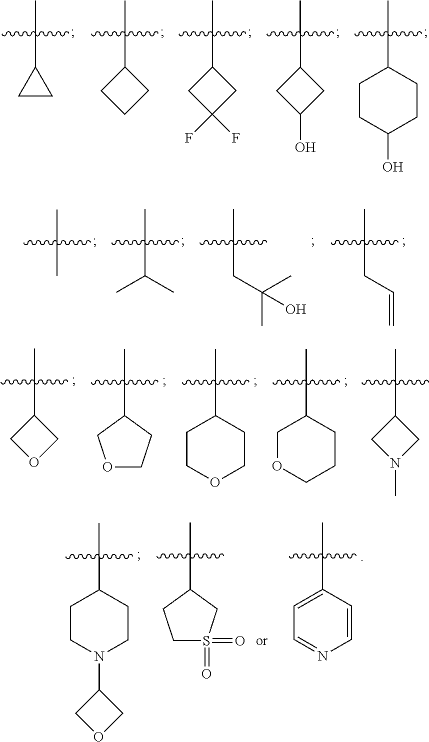

In any of the foregoing embodiments, R^{3a} is oxazolyl, isoxazolyl, 1, 2, 3-oxadiazolyl or 1, 3, 4-oxadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl and C₃-C₈ halocycloalkyl. For example, in certain embodiments, R^{3a} is isoxazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl and C₃-C₈ halocycloalkyl. In further specific embodiments, R^{3a} is substituted with C₁-C₆ alkyl, C₁-C₆ haloalkyl, C₃-C₈ cycloalkyl or C₃-C₈ halocycloalkyl.

In further embodiments, R^{3a} is oxazolyl, isoxazolyl, 1, 2, 3-oxadiazolyl, 1, 3, 4-oxadiazolyl, thiazolyl, isothiazolyl, 1, 2, 4-thiadiazolyl, 1, 3, 4-thiadiazolyl or 1, 2, 4-triazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In certain embodiments, R^{3a} is isoxazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In certain embodiments, R^{3a} is thiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In certain embodiments, R^{3a} is isothiazolyl optionally substituted with one more substituents selected from halo, C₁-C₆ alkyl, C₂-C₆ alkenyl, C₁-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In certain embodiments, R^{3a} is 1,2,4-thiadiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In certain embodiments, R^{3a} is 1,3,4-thiadiazolyl optionally substituted with one more substituents selected from halo, C₁-C₆ alkyl, C₂-C₆ alkenyl, C₁-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In certain embodiments, R^{3a} is 1,3,4-oxadiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, C₁-C₆ aminyl, C₁-C₆ hydroxylalkyl-N, 3-8 membered heterocyclyl and C₃-C₈ halocycloalkyl, or combinations thereof.

In certain embodiments, R^{3a} is 1,2,4-triazolyl optionally substituted with one more substituents selected from halo, C₁-C₆ alkyl, C₂-C₆ alkenyl, C₁-C₆ alkynyl, C₁-C₆ haloalkyl, C₃-C₈ cycloalkyl, cyano, C₁-C₆ aminyl, C₁-C₆ hydroxylalkyl, 3-8 membered heterocyclyl and C₃-C₈ halocycloalkyl, or combinations thereof.

In further embodiments, R^{3a} is substituted with C₁-C₆ alkyl, C₁-C₆ haloalkyl, C₃-C₈ cycloalkyl, cyano, C₁-C₆ aminyl, C₁-C₆ hydroxylalkyl, 3-8 membered heterocyclyl or C₃-C₈ halocycloalkyl, or combinations thereof.

In various embodiments. R^{3a} has one of the following structures:

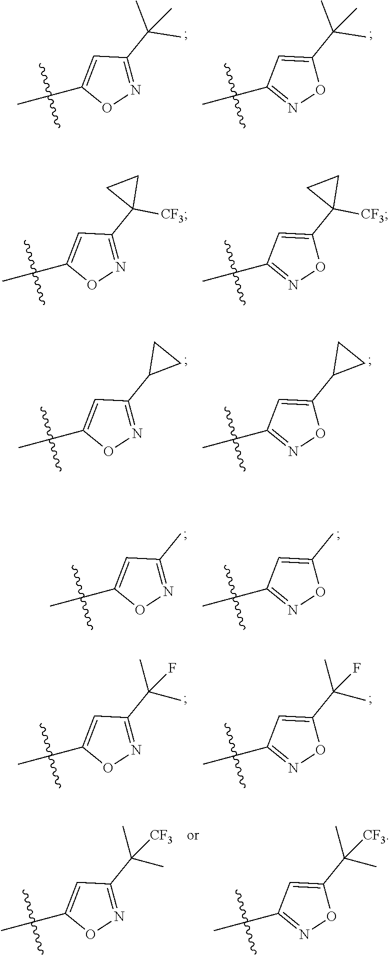

In further embodiments, R^{3a} has one of the following structures:

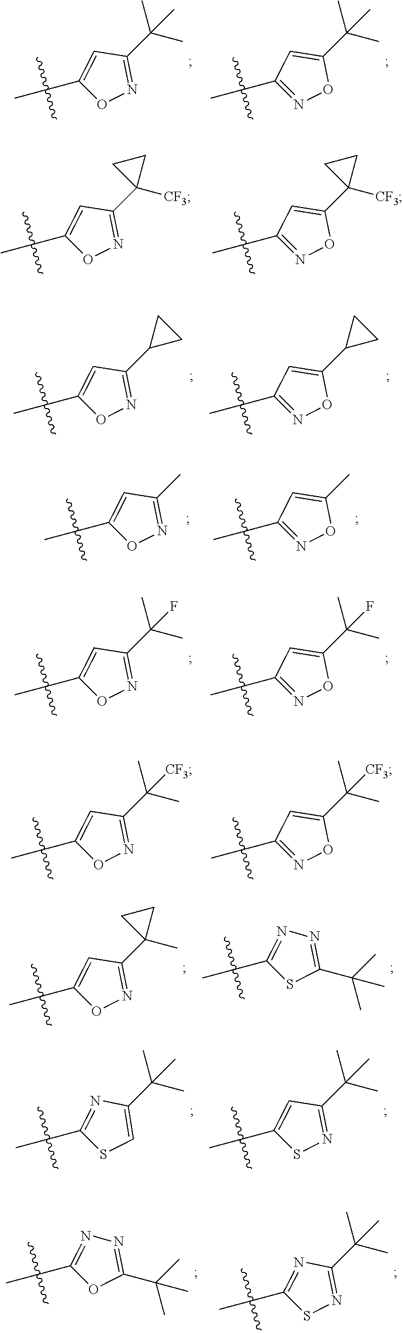

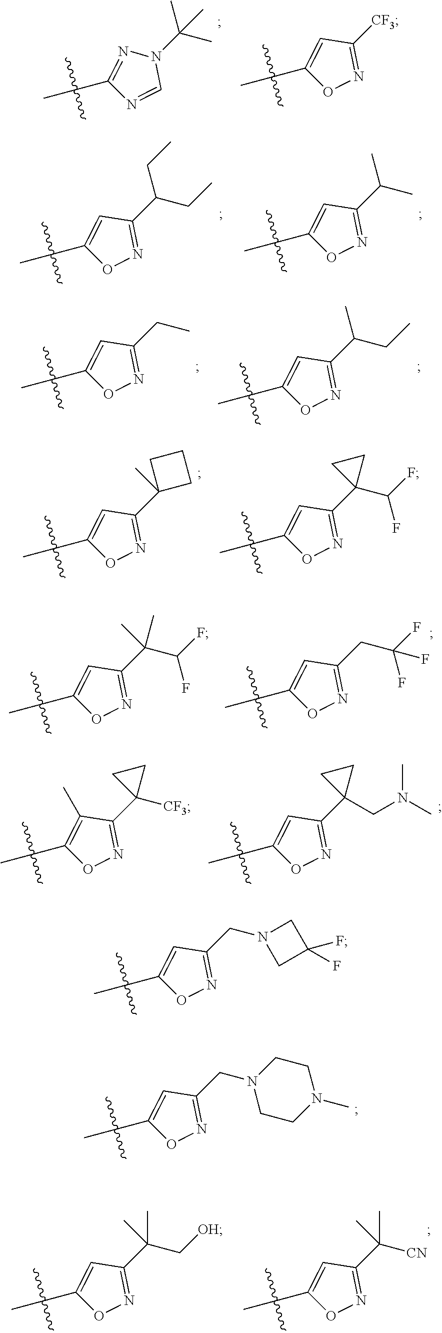

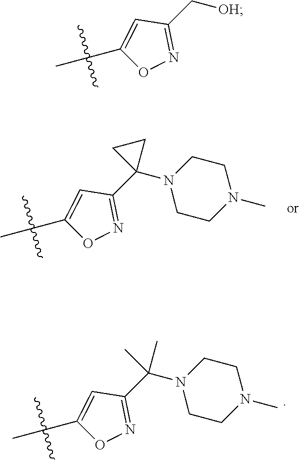 [SMILES: *c1cc(CO)no1 ; *c1cc(C2(N3CCN(C)CC3)CC2)no1 ; *c1cc(C(C)(C)N2CCN(C)CC2)no1]

In other embodiments, R^{4a} is H. In other embodiments, R^{4a} C₁-C₆ alkyl, such as methyl.

In certain embodiments, Y^{a} is CHOH. In other embodiments, Y^{a} is NH.

In other embodiments, X^{a} is N. In more embodiments, X^{a} is CH.

In various embodiments, A1 is C₆-C₁₀ aryl, C₃-C₁₀ cycloalkyl or 5-6 membered monocyclic heteroaryl, each of which is optionally substituted with one or more R^{5a}. It is understood that A1 is a divalent radical.

In certain embodiments, A1 is a divalent optionally substituted C₆₋₁₀ aryl. In certain embodiments, A1 is a divalent optionally substituted 3-8 membered saturated or partially unsaturated carbocyclic ring. In certain embodiments, A1 is a divalent optionally substituted 3-10 membered heterocylic ring having 1-4 heteroatoms independently selected from nitrogen, oxygen, or sulfur. In certain embodiments, A1 is a divalent optionally substituted 5-6 membered monocyclic heteroaryl ring having 1-4 heteroatoms independently selected from nitrogen, oxygen, or sulfur.

In certain embodiments, A1 is a divalent group selected from phenyl, pyridinyl, cyclohexyl, and cyclohexenyl; each of which is optionally substituted.

In other embodiments, A1 is phenyl. In different embodiments, A1 is saturated or unsaturated cyclohexyl. In more embodiments, A1 is pyridinyl.

In further embodiments, A1 is pyrimidinyl, which is optionally substituted. In any of the foregoing embodiments, A1 is unsubstituted. In different of the foregoing embodiments. A1 is substituted with one or more R^{5a}. For example, in some embodiments R^{5a} is halo. In other embodiments, R^{5a} is fluoro. In other different embodiments, R^{5a} is chloro.

In some embodiments, R^{5a} is cyano. In some embodiments, R^{5a} is C₁-C₆ alkyl. In certain embodiments, R^{5a} is methyl. In some embodiments, R^{5a} is C₁-C₆ haloalkyl. In certain embodiments R^{5a} is difluoromethyl. In further embodiments, R^{5a} is C₁-C₆ hydroxylalkyl. In certain embodiments R^{5a} is —CH₂OH.

In certain embodiments, A1 is a divalent group selected from phenyl, naphthyl, cyclopropyl, cyclobutyl, cyclopentyl, cyclohexyl, cyclohexenyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl. NH-carbazolyl, carbolinyl, chromanyl, chromenyl, cinnolinyl, decahydroquinolinyl, dithiazinyl, tetrahydrofuranyl, furanyl, furazanyl, imidazolidinyl, imidazolinyl, imidazolyl, 1H-indazolyl, indolenyl, indolinyl, indolizinyl, indolyl, 3-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2-pyrrolyl, pyrrolyl, quinazolinyl, quinolinyl, 4H-quinolizinyl, quinoxalinyl, quinuclidinyl, tetrahydro furanyl, tetrahydroisoquinolinyl, tetrahydroquinolinyl, thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, and xanthenyl; each of which is optionally substituted.

In specific embodiments, A1 has one of the following structures:

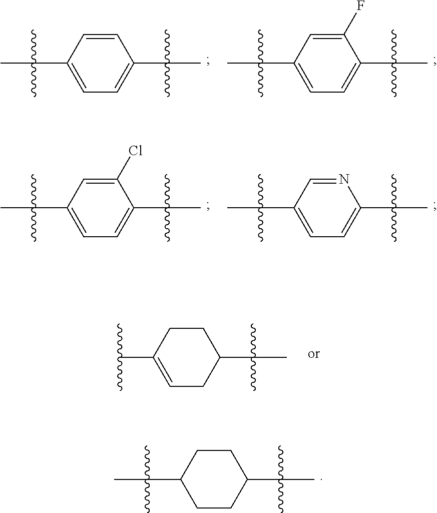 [SMILES: *c1ccc(*)cc1 ; *c1ccc(*)c(F)c1 ; *c1ccc(*)c(Cl)c1 ; *c1ccc(*)nc1 ; *C1=CCC(*)CC1 ; *C1CCC(*)CC1]

In other specific embodiments, A1 has one of the following structures:

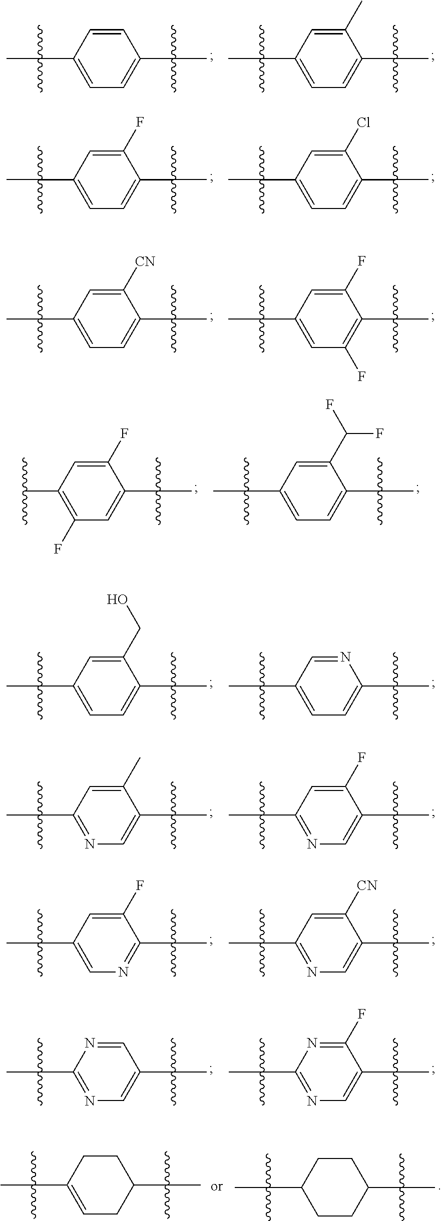

In a certain embodiment, the compound of Structure (Ia) is a modulator of the NLRP3 inflammasome.

In a specific embodiment, the compound of Structure (Ia) is an inhibitor of NEK7 in a patient or in a biological sample.

In various different embodiments, the compound has one of the structures set forth in Table 1a below, or a pharmaceutically acceptable salt, stereoisomer or prodrug thereof. Compounds in Table 1a were prepared as described in the Examples or methods known in the art and analyzed by mass spectrometry and/or ¹H NMR.

TABLE 1a
Representative Compounds of Structure (Ia)

| No. | Structure | Name |
|---|---|---|
| Ia-1 | 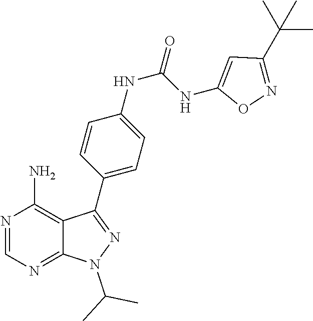 [SMILES: CC(C)n1nc(-c2ccc(NC(=O)Nc3cc(C(C)(C)C)no3)cc2)c2c(N)ncnc21] | 1-(4-(4-amino-1- isopropyl-1H- pyrazolo[3,4- d]pyrimidin-3- yl)phenyl)-3-(3-(tert- butyl)isoxazol-5-yl)urea |
| Ia-2 | 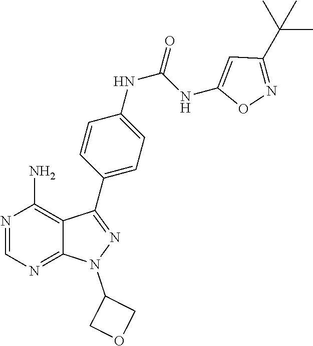 [SMILES: CC(C)(C)c1cc(NC(=O)Nc2ccc(-c3nn(C4COC4)c4ncnc(N)c34)cc2)on1] | 1-(4-(4-amino-1-(oxetan- 3-yl)-1H-pyrazolo(3,4- d]pyrimidin-3- yl)phenyl)-3-(3-(tert- butyl)isoxazol-5-yl)urea |
| Ia-3 | 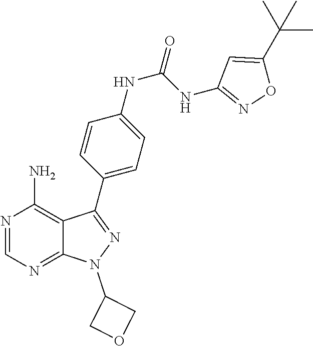 [SMILES: CC(C)(C)c1cc(NC(=O)Nc2ccc(-c3nn(C4COC4)c4ncnc(N)c34)cc2)no1] | 1-(4-(4-amino-(oxetan- 3-yl)-1H-pyrazolo[3,4- d]pyrimidin-3- yl)phenyl)-3-(5-(tert- butyl)isoxazol-3-yl)urea |
| Ia-4 | 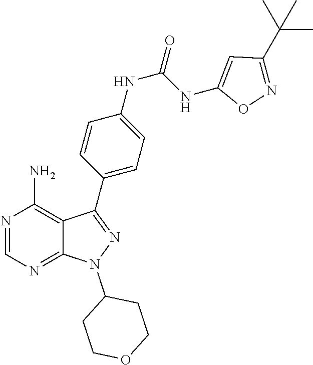 [SMILES: CC(C)(C)c1cc(NC(=O)Nc2ccc(-c3nn(C4CCOCC4)c4ncnc(N)c34)cc2)on1] | 1-(4-(4-amino-1- (tetrahydro-2H-pyran-4- yl)-1H-pyrazolo[3,4- d]pyrimidin-3- yl)phenyl)-3-(3-(tert- butyl)isoxazol-5-yl)urea |
| Ia-5 | 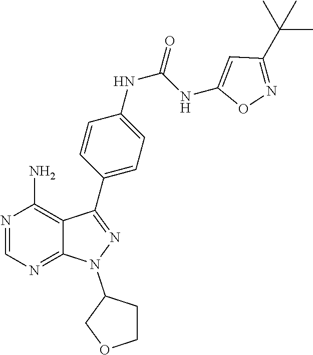 [SMILES: CC(C)(C)c1cc(NC(=O)Nc2ccc(-c3nn(C4CCOC4)c4ncnc(N)c34)cc2)on1] | 1-(4-(4-amino-1- (tetrahydrofuran-3-yl)- 1H-pyrazolo[3,4- d]pyrimidin-3- yl)phenyl)-3-(3-(tert- butyl)isoxazol-5-yl)urea |
| Ia-6 | 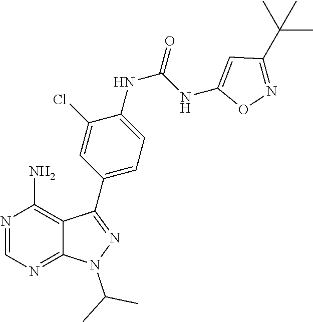 [SMILES: CC(C)n1nc(-c2ccc(NC(=O)Nc3cc(C(C)(C)C)no3)c(Cl)c2)c2c(N)ncnc21] | 1-(4-(4-amino-1- isopropyl-1H- pyrazolo[3,4- d]pyrimidin-3-yl)-2- chlorophenyl)-3-(3-(tert- butyl)isoxazol-5-yl)urea |
| Ia-7 | 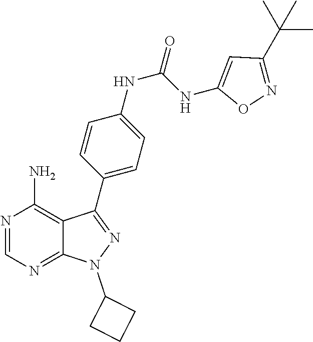 [SMILES: CC(C)(C)c1cc(NC(=O)Nc2ccc(-c3nn(C4CCC4)c4ncnc(N)c34)cc2)on1] | 1-(4-(4-amino-1- cyclobutyl-1H- pyrazolo[3,4- d]pyrimidin-3- yl)phenyl)-3-(3-(tert- butyl)isoxazol-5-yl)urea |
| Ia-8 | 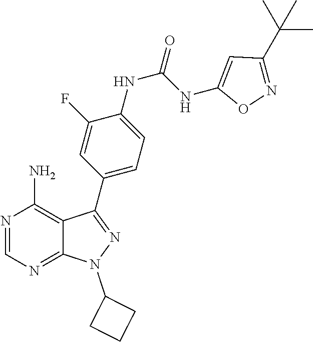 [SMILES: CC(C)(C)c1cc(NC(=O)Nc2ccc(-c3nn(C4CCC4)c4ncnc(N)c34)cc2F)on1] | 1-(4-(4-amino-1- cyclobutyl-1H- pyrazolo[3,4- d]pyrimidin-3-yl)-2- fluorophenyl)-3-(3-(tert- butyl)isoxazol-5-yl)urea |
| Ia-9 | 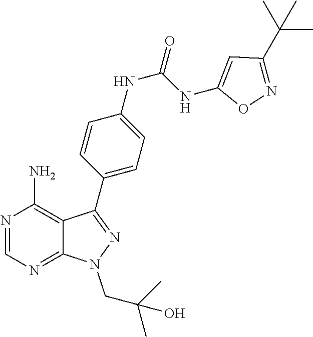 [SMILES: CC(C)(O)Cn1nc(-c2ccc(NC(=O)Nc3cc(C(C)(C)C)no3)cc2)c2c(N)ncnc21] | 1-(4-(4-amino-1-(2- hydroxy-2- methylpropyl)-1H- pyrazolo[3,4- d]pyrimidin-3- yl)phenyl)-3-(3-(tert- butyl)isoxazol-5-yl)urea |
| Ia-10 | 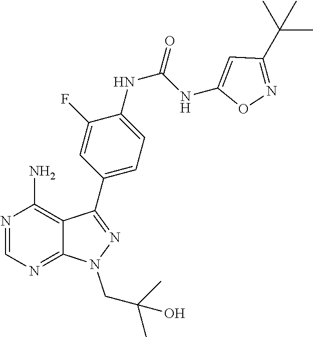 [SMILES: CC(C)(O)Cn1nc(-c2ccc(NC(=O)Nc3cc(C(C)(C)C)no3)c(F)c2)c2c(N)ncnc21] | 1-(4-(4-amino-1-(2- hydroxy-2- methylpropyl)-1H- pyrazolo[3,4- d]pyrimidin-3-yl)-2- fluorophenyl)-3-(3-(tert- butyl)isoxazol-5-yl)urea |
| Ia-11 | 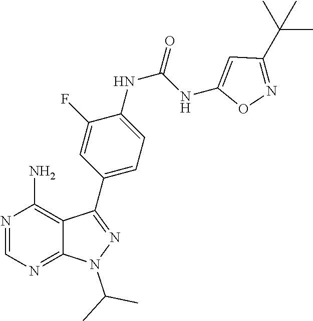 [SMILES: CC(C)n1nc(-c2ccc(NC(=O)Nc3cc(C(C)(C)C)no3)c(F)c2)c2c(N)ncnc21] | 1-(4-(4-amino-1- isopropyl-1H- pyrazolo[3,4- d]pyrirnidin-3-yl)-2- fluorophenyl)-3-(3-(tert- butyl)isoxazol-5-yl)urea |
| Ia-12 | 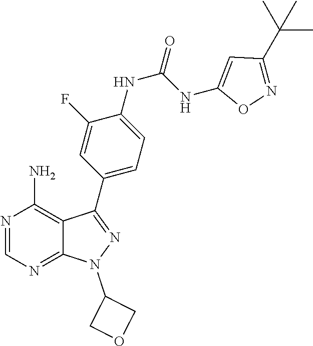 [SMILES: CC(C)(C)c1cc(NC(=O)Nc2ccc(-c3nn(C4COC4)c4ncnc(N)c34)cc2F)on1] | 1-(4-(4-amino-1-(oxetan- 3-yl)-1H-pyrazolo[3,4- d]pyrimidin-3-yl)-2- fluorophenyl)-3-(3-(tert- butyl)isoxazol-5-yl)urea |
| Ia-13 | 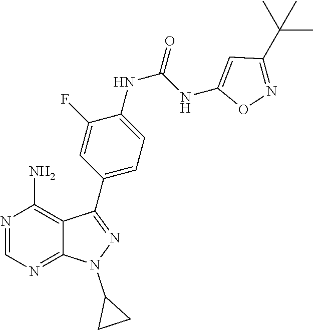 [SMILES: CC(C)(C)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3-(tert- butyl)isoxazol-5-yl)urea |
| Ia-14 | 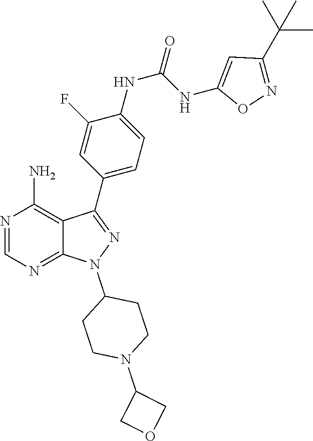 [SMILES: CC(C)(C)c1cc(NC(=O)Nc2ccc(-c3nn(C4CCN(C5COC5)CC4)c4ncnc(N)c34)cc2F)on1] | 1-(4-(4-amino-1-(1- (oxetan-3-yl)piperidin-4- yl)-1H-pyrazolo[3,4- d]pyrimidin-3-yl)-2- fluorophenyl)-3-(3-(tert- butyl)isoxazol-5-yl)urea |
| Ia-15 | 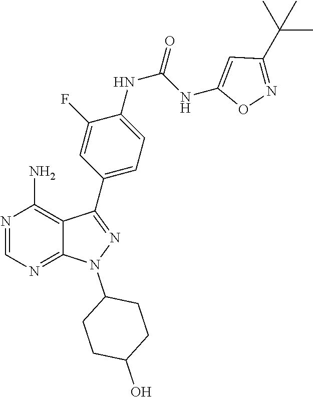 [SMILES: CC(C)(C)c1cc(NC(=O)Nc2ccc(-c3nn(C4CCC(O)CC4)c4ncnc(N)c34)cc2F)on1] | 1-(4-(4-amino-1-(4- hydroxycyclohexyl)-1H- pyrazolo[3,4- d]pyrimidin-3-yl)-2- fluorophenyl)-3-(3-(tert- butyl)isoxazol-5-yl)urea |
| Ia-16 | 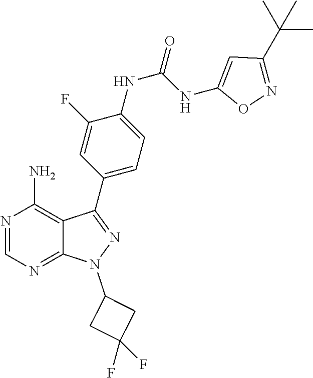 [SMILES: CC(C)(C)c1cc(NC(=O)Nc2ccc(-c3nn(C4CC(F)(F)C4)c4ncnc(N)c34)cc2F)on1] | 1-(4-(4-amino-1-(3,3- difluorocyclobutyl)-1H- pyrazolo[3,4- d]pyrimidin-3-yl)-2- fluorophenyl)-3-(3-(tert- butyl)isoxazol-5-yl)urea |
| Ia-17 | 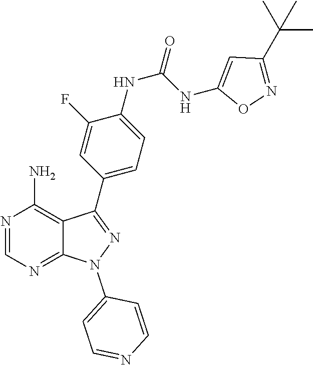 [SMILES: CC(C)(C)c1cc(NC(=O)Nc2ccc(-c3nn(-c4ccncc4)c4ncnc(N)c34)cc2F)on1] | 1-(4-(4-amino-1- (pyridin-4-yl)-1H- pyrazolo[3,4- d]pyrimidin-3-yl)-2- fluorophenyl)-3-(3-(tert- butyl)isoxazol-5-yl)urea |
| Ia-18 | 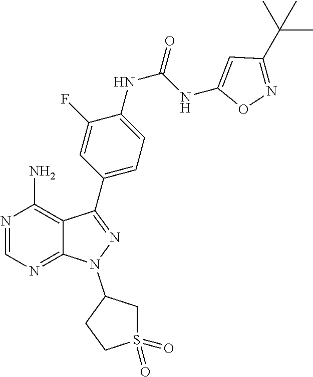 [SMILES: CC(C)(C)c1cc(NC(=O)Nc2ccc(-c3nn(C4CCS(=O)(=O)C4)c4ncnc(N)c34)cc2F)on1] | 1-(4-(4-amino-1-(1,1- dioxidotetrahydrothiophen- 3-yl)-1H-pyrazolo[3,4- d]pyrimidin-3-yl)-2- fluorophenyl)-3-(3-(tert- butyl)isoxazol-5-yl)urea |
| Ia-19 | 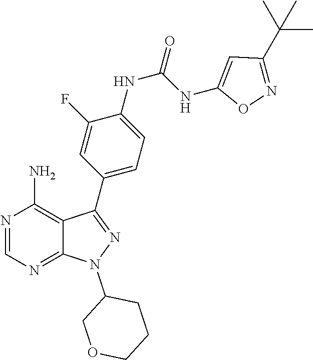 [SMILES: CC(C)(C)c1cc(NC(=O)Nc2ccc(-c3nn(C4CCCOC4)c4ncnc(N)c34)cc2F)on1] | 1-(4-(4-amino-1- (tetrahydro-2H-pyran-3- yl)-1H-pyrazolo[3,4- d]pyrimidin-3-yl)-2- fluorophenyl)-3-(3-(tert- butyl)isoxazol-5-yl)urea |
| Ia-20 | 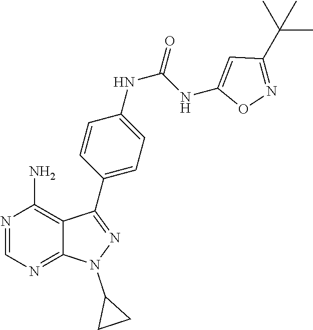 [SMILES: CC(C)(C)c1cc(NC(=O)Nc2ccc(-c3nn(C4CC4)c4ncnc(N)c34)cc2)on1] | 1-(4-(4-amino-1- cyclcpropyl-1H- pyrazolo[3,4- d]pyrimidin-3- yl)phenyl)-3-(3-(tert- butyl)isoxazol-5-yl)urea |
| Ia-21 | 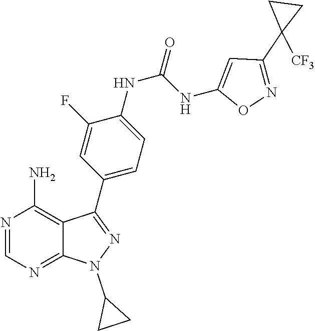 [SMILES: Nc1ncnc2c1c(-c1ccc(NC(=O)Nc3cc(C4(C(F)(F)F)CC4)no3)c(F)c1)nn2C1CC1] | 1-(4-(4-amino-1- cyclopropyl-1H- pyrazolo[3,4- d]pyrimidin-3-yl)-2- fluorophenyl)-3-(3-(1- (trifluoromethyl) cyclopropyl) isoxazol-5-yl)urea |
| Ia-22 | 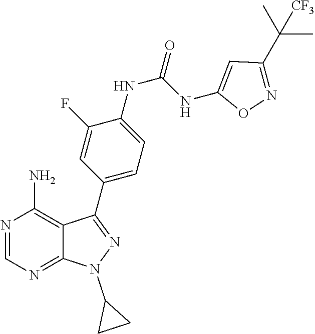 [SMILES: CC(C)(c1cc(NC(=O)Nc2ccc(-c3nn(C4CC4)c4ncnc(N)c34)cc2F)on1)C(F)(F)F] | 1-(4-(4-amino-1- cyclopropyl-1H- pyrazolo[3,4- d]pyrimidin-3-yl)-2- fluorophenyl)-3-(3- (1,1,1-trifluoro-2- methylpropan-2- yl)isoxazol-5-yl)urea |
| Ia-23 | 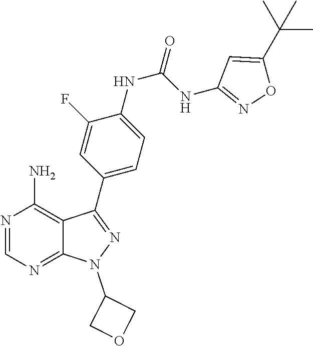 [SMILES: CC(C)(C)c1cc(NC(=O)Nc2ccc(-c3nn(C4COC4)c4ncnc(N)c34)cc2F)no1] | 1-(4-(4-amino-1-(oxetan- 3-yl)-1H-pyrazolo[3,4- d]pyrimidin-3-yl)-2- fluorophenyl)-3-(5-(tert- butyl)isoxazol-3-yl)urea |
| Ia-24 | 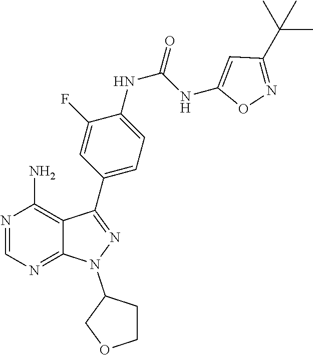 [SMILES: CC(C)(C)c1cc(NC(=O)Nc2ccc(-c3nn(C4CCOC4)c4ncnc(N)c34)cc2F)on1] | 1-(4-(4-amino-1- (tetrahydrofuran-3-yl)- 1H-pyrazolo[3,4- d]pyrimidin-3-yl)-2- fluorophenyl)-3-(3-(tert- butyl)isoxazol-5-yl)urea |
| Ia-25 | 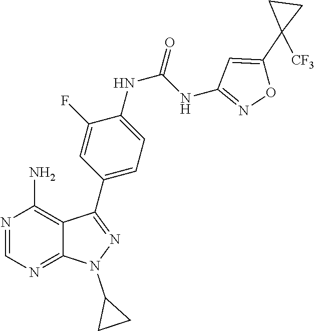 [SMILES: Nc1ncnc2c1c(-c1ccc(NC(=O)Nc3cc(C4(C(F)(F)F)CC4)on3)c(F)c1)nn2C1CC1] | 1-(4-(4-amino-1- cyclopropyl-1H- pyrazolo[3,4- d]pyrimidin-3-yl)-2- fIuorophenyl)-3-(5-(1- (trifluoromethyl) cyclopropyl) isoxazol-3-yl)urea |
| Ia-26 | 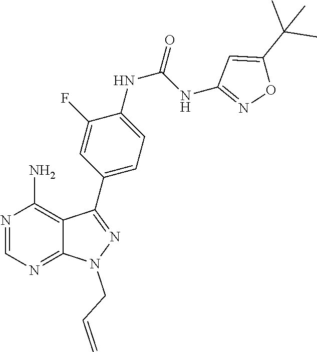 [SMILES: C=CCn1nc(-c2ccc(NC(=O)Nc3cc(C(C)(C)C)on3)c(F)c2)c2c(N)ncnc21] | 1-(4-(1-allyl-4-amino- 1H-pyrazolo[3,4- d]pyrimidin-3-yl)-2- fluorophenyl)-3-(5-(tert- butyl)isoxazol-3-yl)urea |
| Ia-27 | 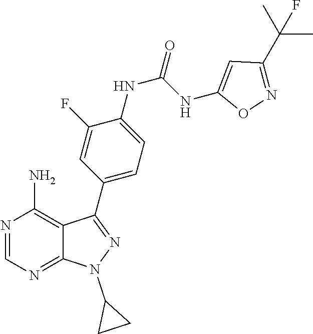 [SMILES: CC(C)(F)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3-(2- fluoropropan-2- yl)isoxazol-5-yl)urea |
| Ia-28 | 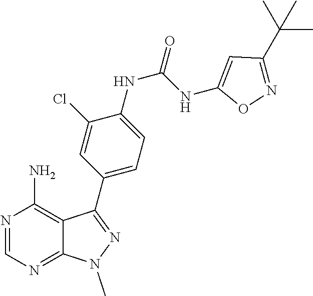 [SMILES: Cn1nc(-c2ccc(NC(=O)Nc3cc(C(C)(C)C)no3)c(Cl)c2)c2c(N)ncnc21] | 1-(4-(4-amino-1-methyl- 1H-pyrazolo[3,4- d]pyrimidin-3-yl)-2- chlorophenyl)-3-(3-(tert- butyl)isoxazol-5-yl)urea |
| Ia-29 | 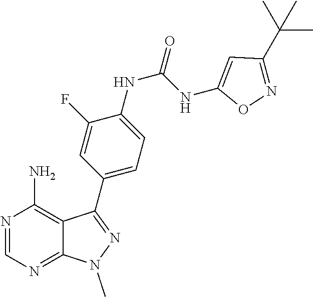 [SMILES: Cn1nc(-c2ccc(NC(=O)Nc3cc(C(C)(C)C)no3)c(F)c2)c2c(N)ncnc21] | 1-(4-(4-amino-1-methyl- 1H-pyrazolo[3,4- d]pyrimidin-3-yl)-2- fluorophenyl)-3-(3-(tert- butyl)isoxazol-5-yl)urea |
| Ia-30 | 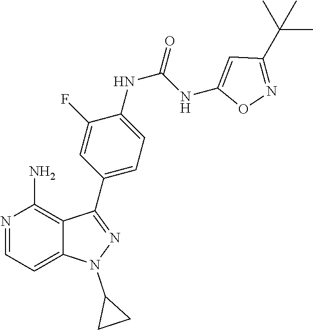 [SMILES: CC(C)(C)c1cc(NC(=O)Nc2ccc(-c3nn(C4CC4)c4ccnc(N)c34)cc2F)on1] | 1-(4-(4-amino-1- cyclopropyl-1H- pyrazolo[4,3-c]pyridin-3- yl)-2-fluorophenyl)-3-(3- (tert-butyl)isoxazol-5- yl)urea |
| Ia-31 | 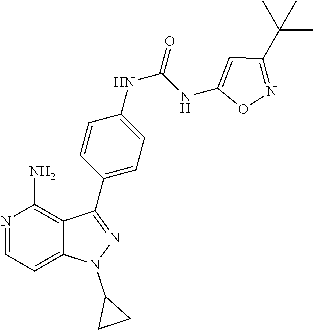 [SMILES: CC(C)(C)c1cc(NC(=O)Nc2ccc(-c3nn(C4CC4)c4ccnc(N)c34)cc2)on1] | 1-(4-(4-amino-1- cyclopropyl-1H- pyrazolo[4,3-c]pyridin-3- yl)phenyl)-3-(3-(tert- butyl)isoxazo1-5-yl)urea |
| Ia-32 | 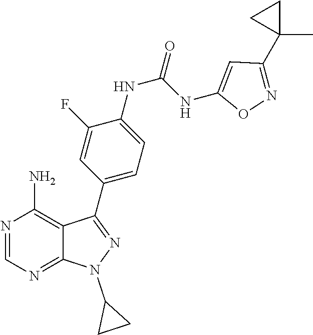 [SMILES: CC1(c2cc(NC(=O)Nc3ccc(-c4nn(C5CC5)c5ncnc(N)c45)cc3F)on2)CC1] | 1-(4-(4-amino-1- cyclopropyl-1H- pyrazolo[3,4- d]pyrimidin-3-yl)-2- fluorophenyl)-3-(3-(1- methylcyclopropyl) isoxazol-5-yl)urea |
| Ia-33 | 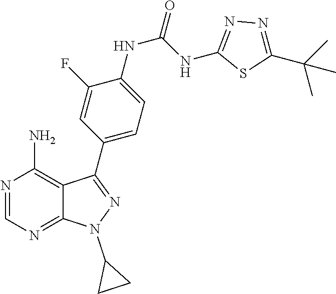 [SMILES: CC(C)(C)c1nnc(NC(=O)Nc2ccc(-c3nn(C4CC4)c4ncnc(N)c34)cc2F)s1] | 1-(4-(4-amino-1- cyclopropyl-1H- pyrazolo[3,4- d]pyrimidin-3-yl)-2- fluoropbenyl)-3-(5-(tert- butyl)-1,3,4-thiadiazol-2- yl)urea |
| Ia-34 | 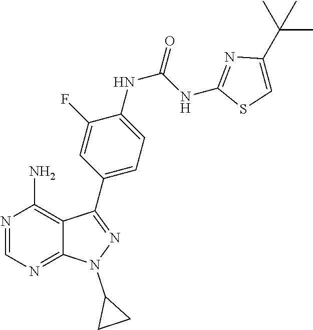 [SMILES: CC(C)(C)c1csc(NC(=O)Nc2ccc(-c3nn(C4CC4)c4ncnc(N)c34)cc2F)n1] | 1-(4-(4-amino-1- cyclopropyl-1H- pyrazolo[3,4- d]pyrimidin-3-yl)-2- fluorophenyl)-3-(4-tert- butyl)thiazol-2-yl)urea |
| Ia-35 | 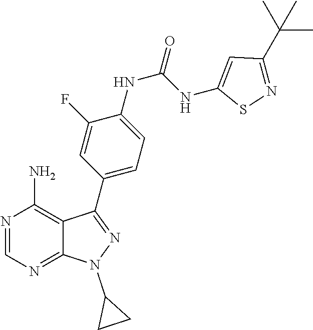 [SMILES: CC(C)(C)c1cc(NC(=O)Nc2ccc(-c3nn(C4CC4)c4ncnc(N)c34)cc2F)sn1] | 1-(4-(4-amino-1- cyclopropyl-1H- pyrazolo[3,4- d]pyrimidin-3-yl)-2- fluorophenyl)-3-(3-(tert- butyl)isothiazol-5- yl)urea |
| Ia-36 | 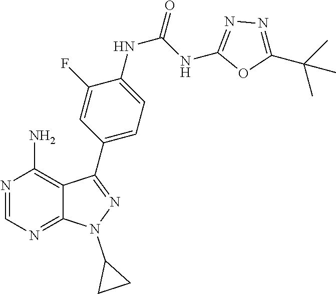 [SMILES: CC(C)(C)c1nnc(NC(=O)Nc2ccc(-c3nn(C4CC4)c4ncnc(N)c34)cc2F)o1] | 1-(4-(4-amino-1- cyclopropyl-1H- pyrazolo[3,4- d]pyrimidin-3-yl)-2- fluorophenyl)-3-(5-(tert- butyl)-1,3,4-oxadiazol-2- yl)urea |
| Ia-37 | 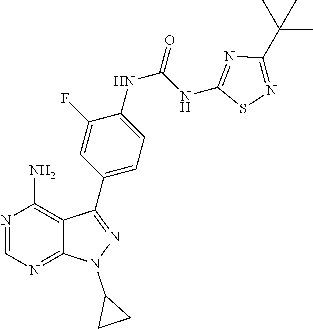 [SMILES: CC(C)(C)c1nsc(NC(=O)Nc2ccc(-c3nn(C4CC4)c4ncnc(N)c34)cc2F)n1] | 1-(4-(4-amino-1- cyclopropyl-1H- pyrazolo[3,4- d]pyrimidin-3-yl)-2- fluorophenyl)-3-(3-(tert- butyl)-1,2,4-thiadiazol-5- yl)urea |
| Ia-38 | 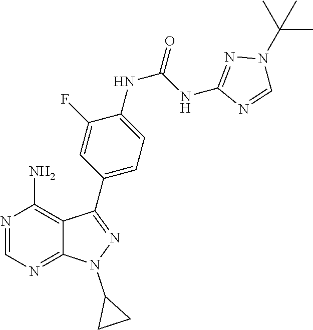 [SMILES: CC(C)(C)n1cnc(NC(=O)Nc2ccc(-c3nn(C4CC4)c4ncnc(N)c34)cc2F)n1] | 1-(4-(4-amino-1- cyclopropyl-1H- pyrazolo[3,4- d]pyrimidin-3-yl)-2- fluorophenyl)-3-(1-(tert- butyl)-1H-1,2,4-triazol- 3-yl)urea |
| Ia-39 | 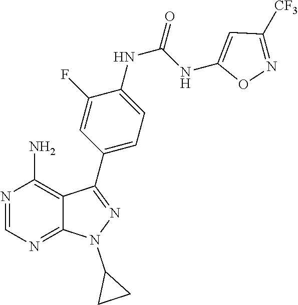 [SMILES: Nc1ncnc2c1c(-c1ccc(NC(=O)Nc3cc(C(F)(F)F)no3)c(F)c1)nn2C1CC1] | 1-(4-(4-amino-1- cyclopropyl-1H- pyrazolo[3,4- d]pyrimidin-3-yl)-2- fluorophenyl)-3-(3- (trifluoromethyl) isoxazol-5-yl)urea |
| Ia-40 | 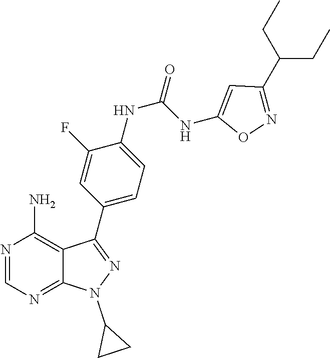 [SMILES: CCC(CC)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3- (pentan-3-yl)isoxazol-5- yl)urea |
| Ia-41 | 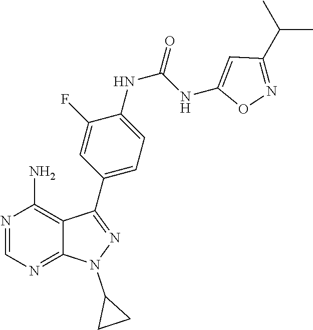 [SMILES: CC(C)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3- isopropylisoxazo1-5- yl)urea |
| Ia-42 | 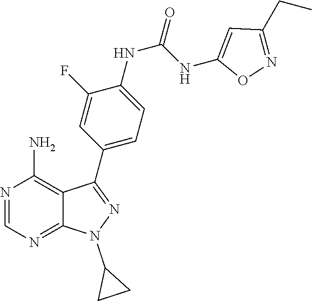 [SMILES: CCc1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3- ethylisoxazol-5- yl)urea |
| Ia-43 | 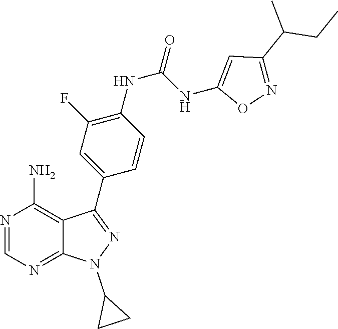 [SMILES: CCC(C)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3-(sec- butyl)isoxazol-5-yl)urea |
| Ia-44 | 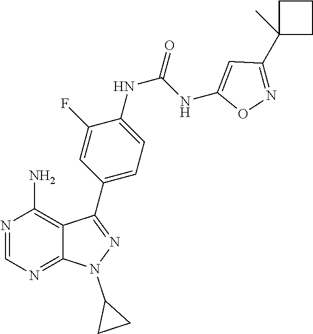 [SMILES: CC1(c2cc(NC(=O)Nc3ccc(-c4nn(C5CC5)c5ncnc(N)c45)cc3F)on2)CCC1] | 1-(4-(4-amino-1- cyclopropyl-1H- pyrazolo[3,4- d]pyrimidin-3-yl)-2- fluorophenyl)-3-(3-(1- methylcyclobutyl) isoxazol-5-yl)urea |
| Ia-45 | 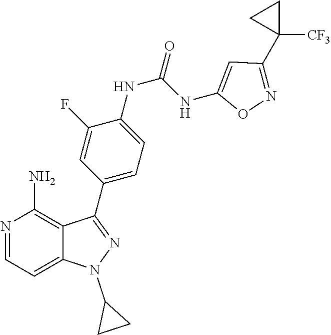 [SMILES: Nc1nccc2c1c(-c1ccc(NC(=O)Nc3cc(C4(C(F)(F)F)CC4)no3)c(F)c1)nn2C1CC1] | 1-(4-(4-amino-1- cyclopropyl-1H- pyrazolo[4,3-c]pyridin-3- yl)-2-fluorophenyl)-3-(3- (1-(trifluoromethyl) cyclopropyl) isoxazol-5-yl)urea |
| Ia-46 | 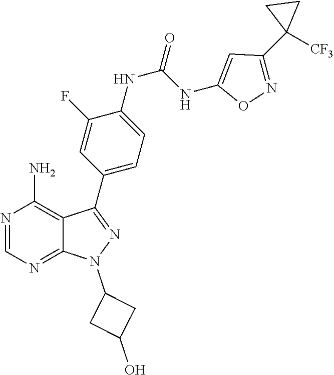 [SMILES: Nc1ncnc2c1c(-c1ccc(NC(=O)Nc3cc(C4(C(F)(F)F)CC4)no3)c(F)c1)nn2C1CC(O)C1] | 1-(4-(4-amino-1-(3- hydroxycyclobutyl)-1H- pyrazolo[3,4- d]pyrimidin-3-yl)-2- fluorophenyl)-3-(3-(1- (trifluoromethyl) cyclopropyl)isoxazol- 5-yl)urea |
| Ia-47 | 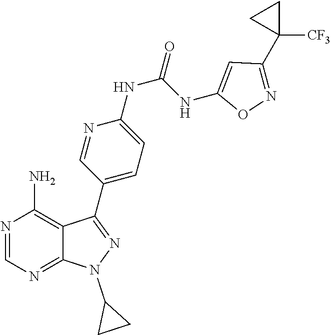 [SMILES: Nc1ncnc2c1c(-c1ccc(NC(=O)Nc3cc(C4(C(F)(F)F)CC4)no3)nc1)nn2C1CC1] | 1-(5-(4-amino-1- cyclopropyl-1H- pyrazolo[3,4- d]pyrimidin-3 yl)pyridin-2-yl)-3-(3-(1- (trifluoromethyl) cyclopropyl)isoxazol-5- yl)urea |
| Ia-48 | 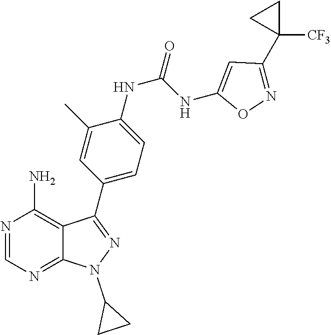 [SMILES: Cc1cc(-c2nn(C3CC3)c3ncnc(N)c23)ccc1NC(=O)Nc1cc(C2(C(F)(F)F)CC2)no1] | 1-(4-(4-amino-1- cyclopropyl-1H- pyrazolo[3,4- d]pyrimidin-3-yl)-2- methylphenyl)-3-(3-(1- (trifluoromethyl) cyclopropyl)isoxazol- 5-yl)urea |
| Ia-49 | 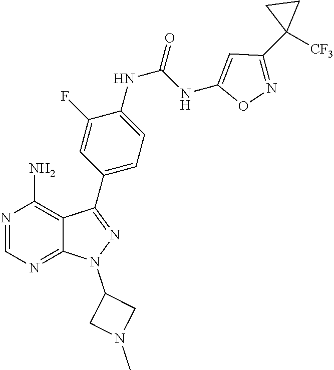 [SMILES: CN1CC(n2nc(-c3ccc(NC(=O)Nc4cc(C5(C(F)(F)F)CC5)no4)c(F)c3)c3c(N)ncnc32)C1] | 1-(4-(4-amino-1-(1- methylazetidin-3-yl)-1H- pyrazolo[3,4- d]pyrimidin-3-yl)-2- flourophenyl)-3-(3-(1- (trifluoromethyl) cyclopropyl) isoxazol-5-yl)urea |
| Ia-50 | 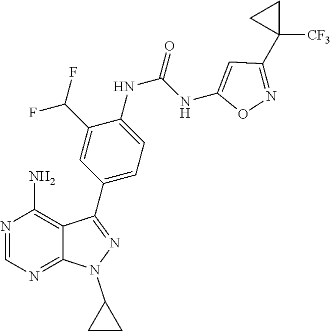 [SMILES: Nc1ncnc2c1c(-c1ccc(NC(=O)Nc3cc(C4(C(F)(F)F)CC4)no3)c(C(F)F)c1)nn2C1CC1] | 1-(4-(4-amino-1- cyclopropyl-1H- pyrazolo[3,4- d]pyrimidin-3-yl)-2- (difluoromethy)phenyl)- 3-(3-1- (trifluoromethyl) cyclopropyl) isoxazol-5-yl)urea |
| Ia-51 | 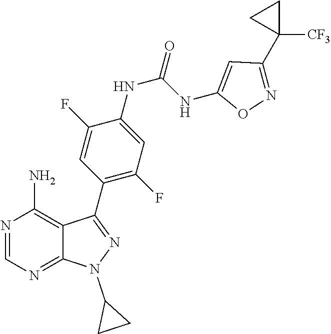 [SMILES: Nc1ncnc2c1c(-c1cc(F)c(NC(=O)Nc3cc(C4(C(F)(F)F)CC4)no3)cc1F)nn2C1CC1] | 1-(4-(4-amino-1- cyclopropyl-1H- pyrazolo[3,4- d]pyrimidin-3-yl)-2,5- difluorophenyl)-3-(3-(1- (trifluoromethyl) cyclopropyl) isoxazol-5-yl)urea |
| Ia-52 | 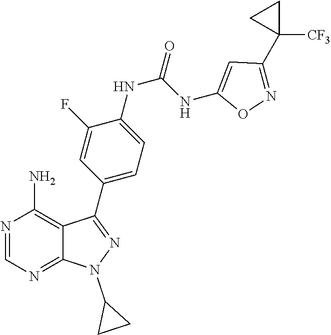 [SMILES: Nc1ncnc2c1c(-c1ccc(NC(=O)Nc3cc(C4(C(F)(F)F)CC4)no3)c(F)c1)nn2C1CC1] | 1-(5-(4-amino-1- cyclopropyl-1H- pyrazolo[3,4- d]pyrimidin-3-yl)-3- flucropyridin-2-yl)-3-(3- (1- (trifluoromethyl) cyclopropyl) isoxazol-5-yl)urea |
| Ia-53 | 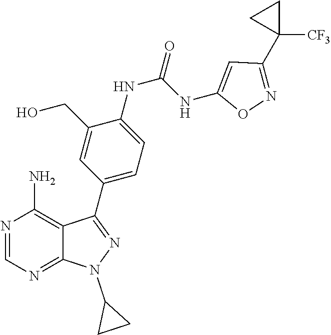 [SMILES: Nc1ncnc2c1c(-c1ccc(NC(=O)Nc3cc(C4(C(F)(F)F)CC4)no3)c(CO)c1)nn2C1CC1] | 1-(4-(4-amino-1- cyclopropyl-1H- pyrazolo[3,4- d]pyrimidin-3-yl)-2- (hydroxymethy)phenyl)- 3-(3-(1- (trifluoromethyl) cyclopropyl) isoxazol-5-yl)urea |
| Ia-54 | 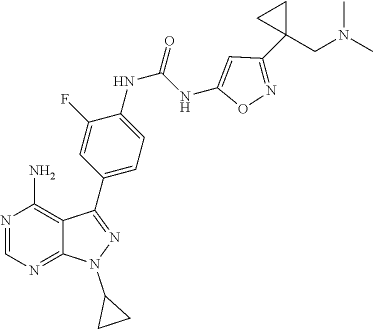 [SMILES: CN(C)CC1(c2cc(NC(=O)Nc3ccc(-c4nn(C5CC5)c5ncnc(N)c45)cc3F)on2)CC1] | 1-(4-(4-amino-1- cyclopropyl-1H- pyrazolo[3,4- d]pyrimidin-3-yl)-2- fluorophenyl)-3-(3-(1- ((dimethylamino)methyl) cyclopropyl)isoxazol-5- yl)urea |
| Ia-55 | 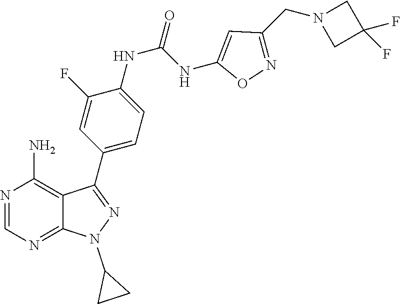 [SMILES: Nc1ncnc2c1c(-c1ccc(NC(=O)Nc3cc(CN4CC(F)(F)C4)no3)c(F)c1)nn2C1CC1] | 1-(4-(4-amino-1- cyclopropyl-1H- pyrazolo[3,4- d]pyrimidin-3-yl)-2- fluorophenyl)-3-(3-((3,3- difluoroazetidin-1- yl)methyl)isoxazol-5- yl)urea |
| Ia-56 | 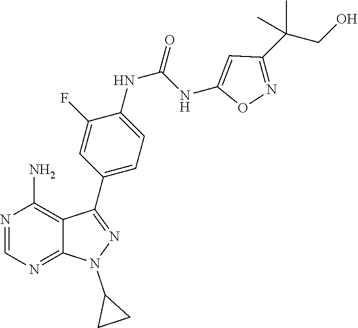 [SMILES: CC(C)(CO)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3-(1- hydroxy-2- methylpropan-2- yl)isoxazol-5-yl)urea |
| Ia-57 | 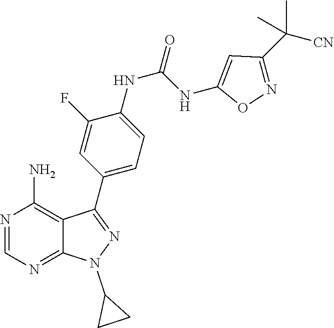 [SMILES: CC(C)(C#N)c1cc(NC(=O)Nc2ccc(-c3nn(C4CC4)c4ncnc(N)c34)cc2F)on1] | 1-(4-(4-amino-1- cyclopropyl-1H- pyrazolo[3,4- d]pyrimidin-3-yl)-2- fluorophenyl)-3-(3-(2- cyanopropan-2- yl)isoxazol-5-yl)urea |
| Ia-58 | 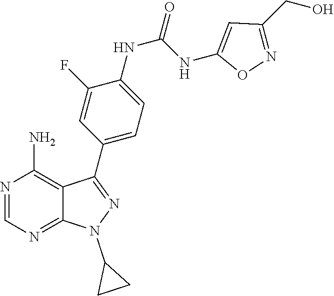 [SMILES: Nc1ncnc2c1c(-c1ccc(NC(=O)Nc3cc(CO)no3)c(F)c1)nn2C1CC1] | 1-(4-(4-amino-1- cyclopropyl-1H- pyrazolo[3,4- d]pyrimidin-3-yl)-2- fluorophenyl)-3-(3- (hydroxymethyl)isoxazol- 5-yl)urea |
| Ia-59 | 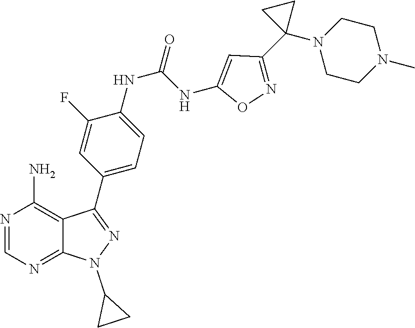 [SMILES: CN1CCN(C2(c3cc(NC(=O)Nc4ccc(-c5nn(C6CC6)c6ncnc(N)c56)cc4F)on3)CC2)CC1] | 1-(4-(4-amino-1- cyclopropyl-1H- pyrazolo[3,4- d]pyrimidin-3-yl)-2- fluorophenyl)-3-(3-(1-(4- methylpiperazin-1- yl)cyclopropyl)isoxazol- 5-yl)urea |
| Ia-60 | 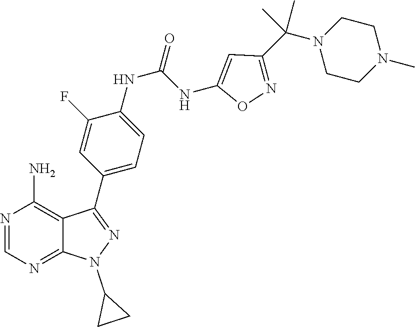 [SMILES: CN1CCN(C(C)(C)c2cc(NC(=O)Nc3ccc(-c4nn(C5CC5)c5ncnc(N)c45)cc3F)on2)CC1] | 1-(4-(4-amino-1- cyclopropyl-1H- pyrazolo[3,4- d]pyrimidin-3-yl)-2- fluorophenyl)-3-(3-(2-(4- methylpiperazin-1- yl)propan-2-yl)isoxazol- 5-yl)urea |
| Ia-61 | 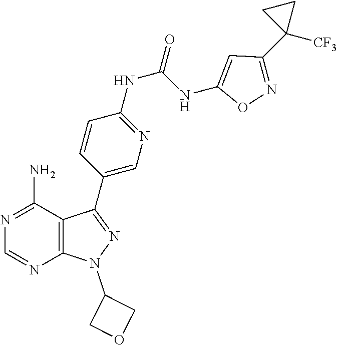 [SMILES: Nc1ncnc2c1c(-c1ccc(NC(=O)Nc3cc(C4(C(F)(F)F)CC4)no3)nc1)nn2C1COC1] | 1-(5-(4-amino-1- cyclopropyl-1H- pyrazolo[4,3-c]pyridin-3- yl)pyridin-2-yl)-3-3-(1- (trifluoromethyl) cyclopropyl) isoxazol-5-yl)urea |

In some embodiments, the NEK7 small molecule inhibitor compound has the following Structure (Ib):

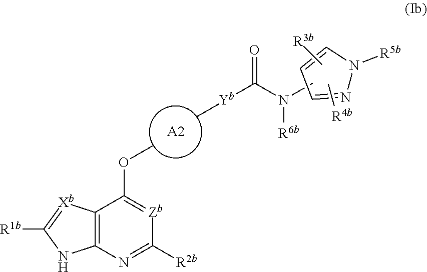

or a pharmaceutically acceptable salt, stereoisomer or prodrug thereof, wherein:

A2 is C₆-C₁₀ aryl, C₃-C₁₀ cycloalkyl, 3-10 membered heterocyclyl, or 5-6 membered monocyclic heteroaryl, each of which is optionally substituted with one or more R^{7b};

X^{b} is N or CR^{10b};

Y^{b} is C(R^{8b})(R^{9b}) or NR^{8b};

Z^{b} is N or CR^{11b};

R^{1b}, R^{2b}, R^{10b}, and R^{11b} are each independently H, halo, C₁-C₆ alkyl, or C₃-C₈ cycloalkyl;

R^{3b} and R^{4b} are each independently H, halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl, provided that R^{2a} and R^{2b} are not both H;

R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy;

R^{6b} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl, each of which is optionally substituted with one more substituents selected from halo, hydroxyl, cyano, aminyl, C₁-C₆ alkyl, C₁-C₆ alkenyl, C₂-C₆ alkynyl, and C₁-C₆ alkoxy;

R^{7b} is, at each occurrence, independently halo, C₁-C₆ alkyl, C₂-C₆ alkenyl, C₂-C₆ alkynyl, or C₁-C₆ haloalkyl;

R^{8b} is H, C₁-C₆ alkyl, C₂-C₆ alkenyl, C₂-C₆ alkynyl, or C₁-C₆ hydroxylalkyl; and

R^{9b} is H, OH, C₁-C₆ alkyl, C₂-C₆ alkenyl, C₂-C₆ alkynyl, or C₁-C₆ hydroxylalkyl.

In some embodiments, A2 is C₆-C₁₀ aryl. In certain embodiments, A2 is C₃-C₁₀ cycloalkyl. In some specific embodiments, A2 is 3-10 membered heterocyclyl. In certain specific embodiments, A2 is 5-6 membered monocyclic heteroaryl. In some more specific embodiments, A2 is substituted with one or more occurrences of R^{7b}. In certain more specific embodiments, A2 is substituted with one or two occurrences of R^{7b}. In some embodiments, A2 is substituted with substituents selected from the group consisting of halo, C₁-C₆ haloalkyl, and combinations thereof. In certain embodiments, A2 is substituted with substituents selected from the group consisting of fluoro, trifluoromethyl, and combinations thereof. In more specific embodiments, A2 is unsubstituted.

In some embodiments, X^{b} is N. In certain embodiments, X^{b} is CR^{10b}. In some specific embodiments, R^{10b} is 1H. In certain more specific embodiments, R^{10b} is halo (e.g., R^{10b} is chloro). In some other embodiments, R^{10b} is C₁-C₆ alkyl (e.g., R^{10b} is methyl). In certain embodiments, R^{10b} is C₃-C₈ cycloalkyl (e.g., R^{10b} is cyclopropyl).

In some embodiments, Z^{b} is N. In other embodiments, Z^{b} is CR^{10b}. In some embodiments, R^{10b} is H. In certain specific embodiments, R^{11b} is halo, C₁-C₆ alkyl, or C₃-C₈ cycloalkyl.

In some embodiments, Y^{b} is C(R^{8b})(R^{9b}). In more specific embodiments, R^{8b} is H. In some embodiments, R^{9b} is —OH, C₁-C₆ alkyl, C₂-C₆ alkenyl, C₂-C₆ alkynyl, or C₁-C₆ hydroxylalkyl. In certain specific embodiments, R^{8b} and R^{9b} are both H.

In some other embodiments, Y^{b} is NR^{8b}. In certain embodiments, R^{8b} is H. In certain other embodiments, R^{8b} is C₁-C₆ alkyl, C₂-C₆ alkenyl, C₂-C₆ alkynyl, or C₁-C₆ hydroxylalkyl.

In certain embodiments, R^{1b} is H. In some embodiments, R^{1b} is C₁-C₆ alkyl (e.g., R^{1b} is methyl). In some embodiments, R^{1b} is halo or C₃-C₈ cycloalkyl.

In some embodiments, R^{2b} is H. In certain embodiments, R^{2b} is halo, C₁-C₆ alkyl, or C₃-C₈ cycloalkyl.

In certain embodiments, R^{3b} is C₁-C₆ alkyl (e.g., R^{3b} is tert-butyl or methyl). In some embodiments, R^{3b} is C₃-C₈ cycloalkyl. In more specific embodiments, R^{3b} is cyclopropyl. In some embodiments, the cyclopropyl is unsubstituted. In some embodiments, the cyclopropyl is substituted with at least one haloalkyl (e.g., trifluoromethyl). In certain more specific embodiments, R^{3b} has the following structure:

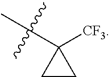 [SMILES: *C1(C(F)(F)F)CC1]

In some embodiments, R^{4b} is H. In certain embodiments, R⁴ is halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl.

In certain specific embodiments, R^{5b} is C₁-C₆ alkyl (e.g., R^{5b} is methyl).

In some embodiments, R^{5b} is aryl. For example, in some embodiments, R^{5b} is phenyl. In certain embodiments, the phenyl is substituted with one or more substituents selected from halo, hydroxyl, cyano, aminyl, C₁-C₆ alkyl, C₂-C₆ alkenyl, C₂-C₆ alkynyl, and C₁-C₆ alkoxy. In certain other embodiments, the phenyl is unsubstituted.

In some other embodiments, R^{5b} is 3-10 membered heterocyclyl. In more specific embodiments, R^{5b} is piperidinyl. In certain specific embodiments, the piperidinyl is substituted with one or more substituents selected from halo, hydroxyl, cyano, aminyl, C₁-C₆ alkyl, C₂-C₆ alkenyl, C₂-C₆ alkynyl, and C₁-C₆ alkoxy. In certain embodiments, R^{5b} has the following structure:

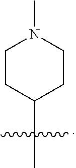 [SMILES: *C1CCN(C)CC1]

In some embodiments, R^{6b} is H. In more specific embodiments, R^{6b} is C₁-C₆ alkyl, C₂-C₆, alkenyl, C₂-C₆ alkynyl, C₃-C₈ cycloalkyl, 3-8 membered heterocyclyl, C₆-C₁₀ aryl, or 5- or 6-membered heteroaryl.

In various different embodiments, the compound has one of the structures set forth in Table 1b below, or a pharmaceutically acceptable salt, stereoisomer, or prodrug thereof. Compounds in Table 1b were prepared as described in the Examples or methods known in the art and analyzed by mass spectrometry and/or ¹H NMR.

Representative compounds of Structure (Ib)

| Compound | Structure | Name |
|---|---|---|
| Ib-1 | 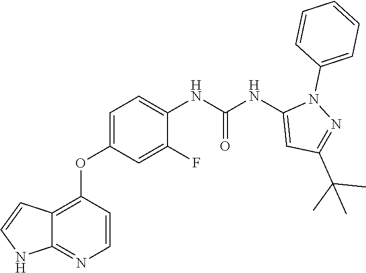 [SMILES: CC(C)(C)c1cc(NC(=O)Nc2ccc(Oc3ccnc4[nH]ccc34)cc2F)n(-c2ccccc2)n1] | 1-(4-((1H-pyrrolo[2,3- b]pyridin-4-yl)oxy)-2- fluorophenyl)-3-(3-(tert- butyl)-1-phenyl-1H-pyrazol- 5-yl)urea |
| Ib-2 | 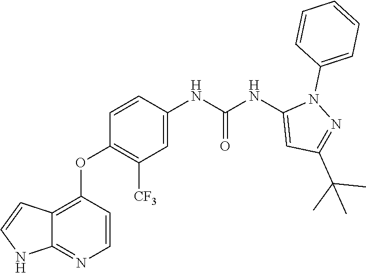 [SMILES: CC(C)(C)c1cc(NC(=O)Nc2ccc(Oc3ccnc4[nH]ccc34)c(C(F)(F)F)c2)n(-c2ccccc2)n1] | 1-(4-((1H-pyrrolo[2,3- b]pyridin-4-yl)oxy)-3- (trifluoromethyl)phenyl)-3-(3- (tert-butyl)-1-phenyl-1H- pyrazol-5-yl)urea |
| Ib-3 | 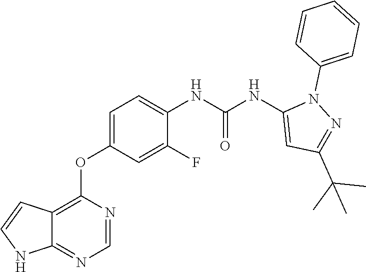 [SMILES: CC(C)(C)c1cc(NC(=O)Nc2ccc(Oc3ncnc4[nH]ccc34)cc2F)n(-c2ccccc2)n1] | 1-(4-((7H-pyrrolo[2,3- d]pyrimidin-4-yl)oxy)-2- fluorophenyl)-3-(3-(tert- butyl)-1-phenyl-1H-pyrazol- 5-yl)urea |
| Ib-4 | 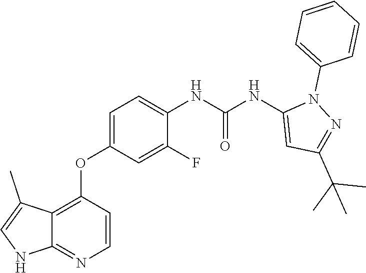 [SMILES: Cc1c[nH]c2nccc(Oc3ccc(NC(=O)Nc4cc(C(C)(C)C)nn4-c4ccccc4)c(F)c3)c12] | 1-(3-(tert-butyl)-1-phenyl-1H- pyrazol-5-yl)-3-(2-fluoro-4- ((3-methyl-1H-pyrrolo[2,3- b]pyridin-4- yl)oxy)phenyl)urea |
| Ib-5 | 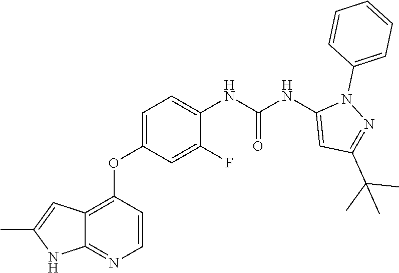 [SMILES: Cc1cc2c(Oc3ccc(NC(=O)Nc4cc(C(C)(C)C)nn4-c4ccccc4)c(F)c3)ccnc2[nH]1] | 1-(3-(tert-butyl)-1-phenyl-1H- pyrazol-5-yl)-3-(2-fluoro-4- ((2-methyl-1H-pyrrolo[2,3- b]pyridin-4- yl)oxy)phenyl)urea |
| Ib-6 | 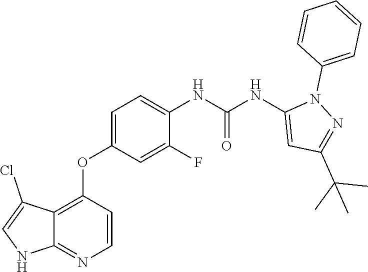 [SMILES: CC(C)(C)c1cc(NC(=O)Nc2ccc(Oc3ccnc4[nH]cc(Cl)c34)cc2F)n(-c2ccccc2)n1] | 1-(3-(tert-butyl)-1-phenyl-1H- pyrazol-5-yl)-3-(4-((3-chloro- 1H-pyrrolo[2,3-b]pyridin-4- yl)oxy)-2-fluorophenyl)urea |
| Ib-7 | 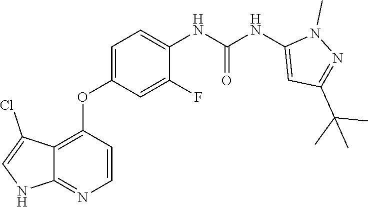 [SMILES: Cn1nc(C(C)(C)C)cc1NC(=O)Nc1ccc(Oc2ccnc3[nH]cc(Cl)c23)cc1F] | 1-(3-(tert-butyl)-1-methyl-1H- pyrazol-5-yl)-3-(4-((3-chloro- 1H-pyrrolo[2,3-b]pyridin-4- yl)oxy)-2-fluorophenyl)urea |
| Ib-8 | 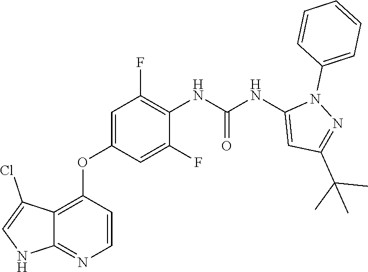 [SMILES: CC(C)(C)c1cc(NC(=O)Nc2c(F)cc(Oc3ccnc4[nH]cc(Cl)c34)cc2F)n(-c2ccccc2)n1] | 1-(3-(tert-butyl)-1-phenyl-1H- pyrazol-5-yl)-3-(4-((3-chloro- 1H-pyrrolo(2,3-b]pyridin-4- yl)oxy)-2,6- difluorophenyl)urea |
| Ib-9 | 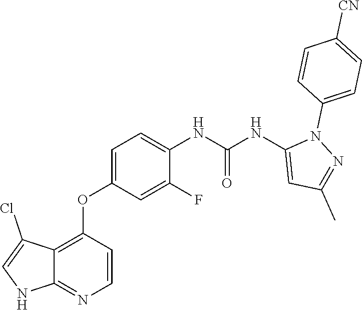 [SMILES: Cc1cc(NC(=O)Nc2ccc(Oc3ccnc4[nH]cc(Cl)c34)cc2F)n(-c2ccc(C#N)cc2)n1] | 1-(4-((3-chloro-1H- pyrrolo[2,3-b]pyridin-4- yl)oxy)-2-fluorophenyl)-3-(1- (4-cyanophenyl)-3-methyl- 1H-pyrazol-5-yl)urea |
| Ib-10 | 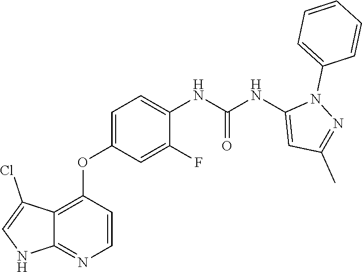 [SMILES: Cc1cc(NC(=O)Nc2ccc(Oc3ccnc4[nH]cc(Cl)c34)cc2F)n(-c2ccccc2)n1] | 1-(4-((3-chloro-1H- pyrrolo[2,3-b]pyridin-4- yd)oxy)-2-fluorophenyl)-3-(3- methyl-1-phenyl-1H-pyrazol- 5-yl)urea |
| Ib-11 | 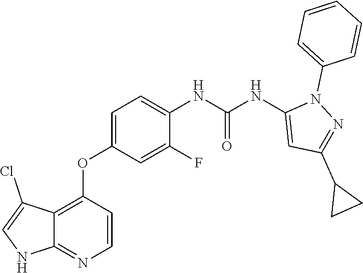 [SMILES: O=C(Nc1ccc(Oc2ccnc3[nH]cc(Cl)c23)cc1F)Nc1cc(C2CC2)nn1-c1ccccc1] | 1-(4-((3-chloro-1H- pyrrolo[2,3-b]pyridin-4- yl)oxy)-2-fluorophenyl)-3-(3- cyclopropyl-1-phenyl-1H- pyrazol-5-yl)urea |
| Ib-12 | 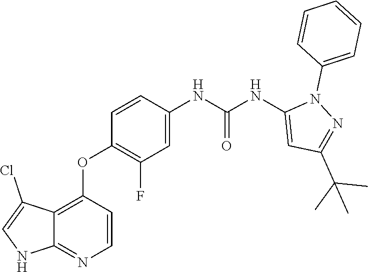 [SMILES: CC(C)(C)c1cc(NC(=O)Nc2ccc(Oc3ccnc4[nH]cc(Cl)c34)c(F)c2)n(-c2ccccc2)n1] | 1-(3-(tert-butyl)-1-phenyl-1H- pyrazol-5-yl)-3-(4-((3-chloro- 1H-pyrrolo[2,3-b]pyridin-4- yl)oxy)-3-fluorophenyl)urea |
| Ib-13 | 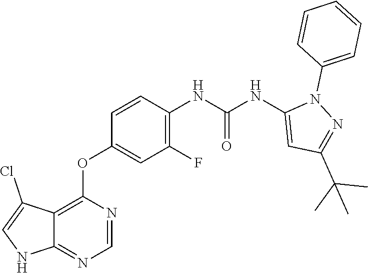 [SMILES: CC(C)(C)c1cc(NC(=O)Nc2ccc(Oc3ncnc4[nH]cc(Cl)c34)cc2F)n(-c2ccccc2)n1] | 1-(3-(tert-butyl)-1-phenyl-1H- pyrazol-5-yl)-3-(4-((5-chloro- 7H-pyrrolo[2,3-d]pyrimidin- 4-yl)oxy)-2-fluorophenyl)urea |
| Ib-14 | 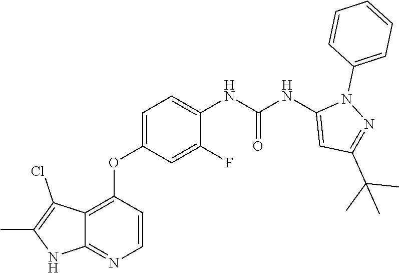 [SMILES: Cc1[nH]c2nccc(Oc3ccc(NC(=O)Nc4cc(C(C)(C)C)nn4-c4ccccc4)c(F)c3)c2c1Cl] | 1-(3-(tert-butyl)-1-phenyl-1H- pyrazol-5-yl)-3-(4-((3-chloro- 2-methyl-1H-pynolo(2,3- b]pyridin-4-yl)oxy)-2- fluorophenyl)urea |
| Ib-15 | 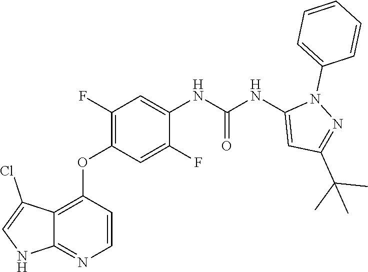 [SMILES: CC(C)(C)c1cc(NC(=O)Nc2cc(F)c(Oc3ccnc4[nH]cc(Cl)c34)cc2F)n(-c2ccccc2)n1] | 1-(3-(tert-butyl)-1-phenyl-1H- pyrazol-5-yl)-3-(4-((3-chloro- 1H-pyrrolo[2,3-b]pyridin-4- yl)oxy)-2,5- difluorophenyl)urea |
| Ib-16 | 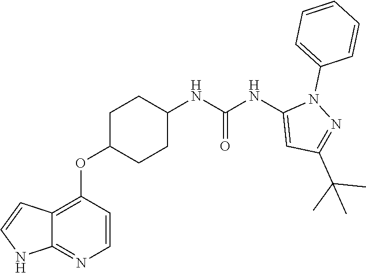 [SMILES: CC(C)(C)c1cc(NC(=O)NC2CCC(Oc3ccnc4[nH]ccc34)CC2)n(-c2ccccc2)n1] | 1-(4-((1H-pyrrolo[2,3- b]pyridin-4- y))oxy)cyclohexyl)-3-(3-(tert- butyl)-1-phenyl-1H-pyrazol- 5-yl)urea |
| Ib-17 | 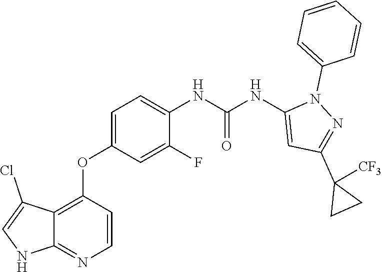 [SMILES: O=C(Nc1ccc(Oc2ccnc3[nH]cc(Cl)c23)cc1F)Nc1cc(C2(C(F)(F)F)CC2)nn1-c1ccccc1] | 1-(4-((3-chloro-1H- pyrrolo[2,3-b]pyridin-4- yl)oxy)-2-fluorophenyl)-3-(1- phenyl-3-(1- (trifluoromethyl)cyclopropyl)- 1H-pyrazol-5-yl)urea |
| Ib-18 | 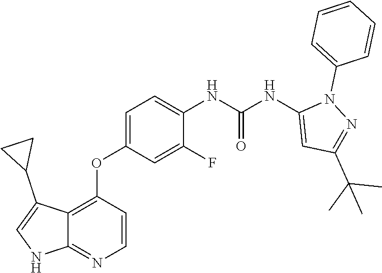 [SMILES: CC(C)(C)c1cc(NC(=O)Nc2ccc(Oc3ccnc4[nH]cc(C5CC5)c34)cc2F)n(-c2ccccc2)n1] | 1-3-(tert-butyl)-1-phenyl-1H- pyrazol-5-yl)-3-(4-((3- cyclopropyl-1H-pyrrolo[2,3- b]pyridin-4-yl)oxy)-2- fluorophenyl)urea |
| Ib-19 | 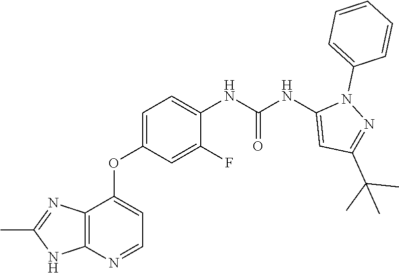 [SMILES: Cc1nc2c(Oc3ccc(NC(=O)Nc4cc(C(C)(C)C)nn4-c4ccccc4)c(F)c3)ccnc2[nH]1] | 1-(3-(tert-butyl)-1-phenyl-1H- pyrazol-5-yl)-3-(2-fluoro-4- ((2-methyl-3H-imidazo[4,5- b]pyridin-7- yl)oxy)phenyl)urea |
| Ib-20 | 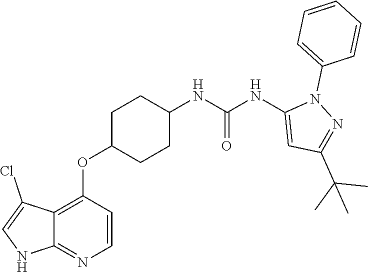 [SMILES: CC(C)(C)c1cc(NC(=O)NC2CCC(Oc3ccnc4[nH]cc(Cl)c34)CC2)n(-c2ccccc2)n1] | 1-(3-tert-butyl)-1-phenyl-1H- pyrazol-5-yl)-3-(4-((3-chloro- 1H-pyrrolo[2,3-b]pyridin-4- yl)oxy)cyclohexyl)urea |
| Ib-21 | 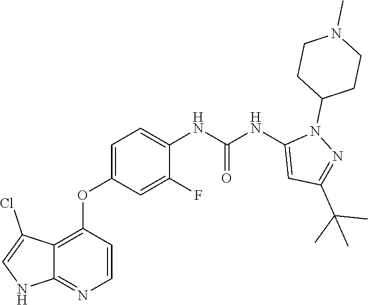 [SMILES: CN1CCC(n2nc(C(C)(C)C)cc2NC(=O)Nc2ccc(Oc3ccnc4[nH]cc(Cl)c34)cc2F)CC1] | 1-(3-(tert-butyl)-1-(1- methylpiperidin-4-yl)-1H- pyrazol-5-yl)-3-(4-((3-chloro- 1H-pyrrolo[2,3-b]pyridin-4- yl)oxy)-2-fluorophenyl)urea |
| Ib-22 | 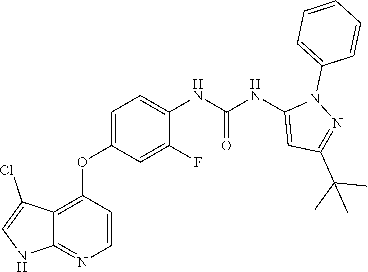 [SMILES: CC(C)(C)c1cc(NC(=O)Nc2ccc(Oc3ccnc4[nH]cc(Cl)c34)cc2F)n(-c2ccccc2)n1] | N-(3-(tert-butyl)-1-phenyl- 1H-pyrazol-5-yl)-2-(4-((3- chloro-1H-pyrrolo[2,3- b]pyridin-4-yl)oxyl-2- fluorophenyl)acetamide |

In some embodiments, the NEK7 small molecule inhibitor compound has the following Structure (Ic):

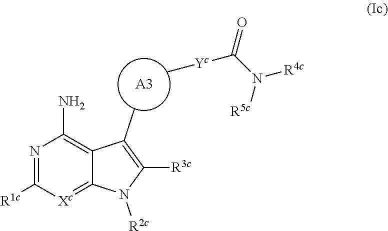

or a pharmaceutically acceptable salt, stereoisomer or prodrug thereof, wherein:

A3 is C₆-C₁₀ aryl, C₃-C₁₀ cycloalkyl, 3-10 membered heterocyclyl or 5-6 membered monocyclic heteroaryl, each of which is optionally substituted with one or more R^{6c};

X^{c} is CH or N;

Y^{c} is CHOH or NH;

R^{1c} is H or C₁-C₆ alkyl;

R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl and C₁-C₆ alkoxy;

R^{4c} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl;

R^{5c} is H, C₁-C₆ alkyl, C₂-C₆ alkenyl, C₂-C₆ alkynyl, C₃-C₈ cycloalkyl, 3- to 8-membered heterocyclyl, C₆-C₁₀ aryl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl and C₁-C₆ alkoxy; and

R^{6c} is, at each occurrence, independently halo, C₁-C₆ alkyl, C₁-C₆ alkoxy, cyano, C₁-C₆ hydroxylalkyl or C₁-C₆ haloalkyl.

In certain embodiment, R^{1c} is H. In other embodiments, R^{1c} C₁-C₆ alkyl, such as methyl.

In one embodiment, compounds of Structure (Ic) are provided, where R^{2e} is branched C₄-C₆ alkyl, C₃-C₄ cycloalkyl, C₃-C₈ heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl.

In another embodiment, compounds of Structure (Ic) are provided, where R^{2c} is branched C₄-C₆ alkyl, C₃-C₄ cycloalkyl, or C₃-C₈ heterocyclyl, each of which is optionally substituted with one more substituent selected from halo, hydroxyl, cyano, aminyl, C₁-C₆ alkyl, C₁-C₆ alkenyl, C₁-C₆ alkynyl, C₁-C₆ alkoxy and 3- to 8-membered heterocyclyl.

In specific embodiments, R^{2c} is cyclopropyl or oxetanyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl. In some embodiments. R^{2c} is cyclopropyl. In other embodiments, R^{2c} is oxetanyl. In some embodiments, R^{2c} is unsubstituted cyclopropyl or oxetanyl.

In specific embodiments, R^{2c} is cyclopropyl, cyclobutyl, pyrrolidinyl, piperidinyl, or oxetanyl, each of which is optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl. In some embodiments, R^{2c} is cyclopropyl.

In other embodiments, R^{2c} is oxetanyl. In some embodiments, R^{2c} is unsubstituted cyclopropyl or oxetanyl. In some embodiments, R^{2c} is N-methyl substituted pyrrolidinyl.

In certain specific embodiments, R^{2c} is unsubstituted cyclobutyl.

In different embodiments, R^{2c} is branched C₄-C₆ alkyl optionally substituted with one more substituent selected from halo, hydroxyl, cyano, aminyl, C₁-C₆ alkyl, C₂-C₆ alkenyl, C₂-C₆ alkynyl, C₁-C₆ alkoxy and 3- to 8-membered heterocyclyl. For example, in some embodiments R^{2c} is 2-methylpropyl optionally substituted with hydroxyl.

In more specific embodiments, R^{2c} has one of the following structures:

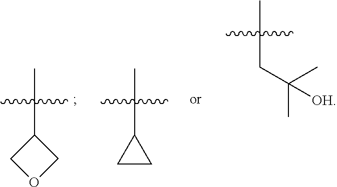

In some specific embodiments, R^{2c} has one of the following structures:

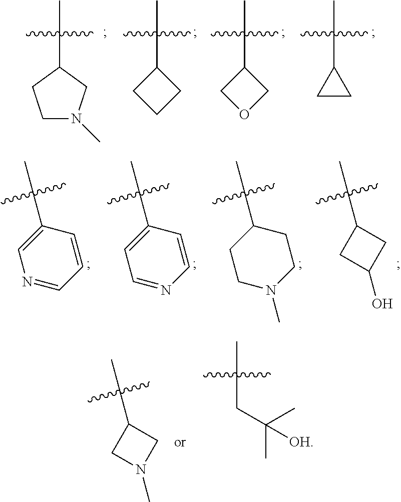

In other embodiments, R^{3c} is H. In other embodiments, R^{3c} C₁-C₆ alkyl, such as methyl.

In any of the foregoing embodiments, R^{4C} is oxazolyl, isoxazolyl, 1, 2, 3-oxadiazolyl or 1, 3, 4-oxadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof. For example, in certain embodiments, R^{4c} is isoxazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl and C₃-C₈ halocycloalkyl. In further specific embodiments, R^{4c} is substituted with C₁-C₆ alkyl, C₁-C₆ haloalkyl, C₃-C₈ cycloalkyl or C₃-C₈ halocycloalkyl.

In certain embodiments, R^{4c} is oxazolyl, isoxazolyl, 1, 2, 3-oxadiazolyl, thiazolyl, isothiazolyl, 1,2,4-thiadiazolyl, 1,3,4-thiadiazolyl, 1,2,4-triazolyl or 1, 3, 4-oxadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₁-C₈ aminylalkylcycloalkyl, C₁-C₆ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments, R^{4c} is isoxazolyl optionally substituted with one more substituents selected from halo, C₁-C₆ alkyl, C₂-C₆ alkenyl, C₁-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments, R^{4c} is thiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₁-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments. R^{4c} is isothiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments, R^{4c} is 1,2,4-thiadiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments, R^{4c} is 1,3,4-thiadiazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments, R^{4c} is 1,2,4-triazolyl optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In certain embodiments, R^{4c} is 1, 3, 4-oxadiazolyl optionally substituted with one more substituents selected from halo, C₁-C₆ alkyl, (C-G alkenyl, C₂-C₆ alkynyl, C₁-C₆ haloalkyl, C₃-C₈ cycloalkyl, cyano, aminyl, C₁-C₆ hydroxylalkyl, 3- to 8-membered heterocyclyl and C₃-C₈ halocycloalkyl, or combinations thereof.

In certain embodiments, R^{4c} is substituted with C₁-C₆ alkyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl, and combinations thereof.

In various embodiments, R^{4c} has one of the following structures:

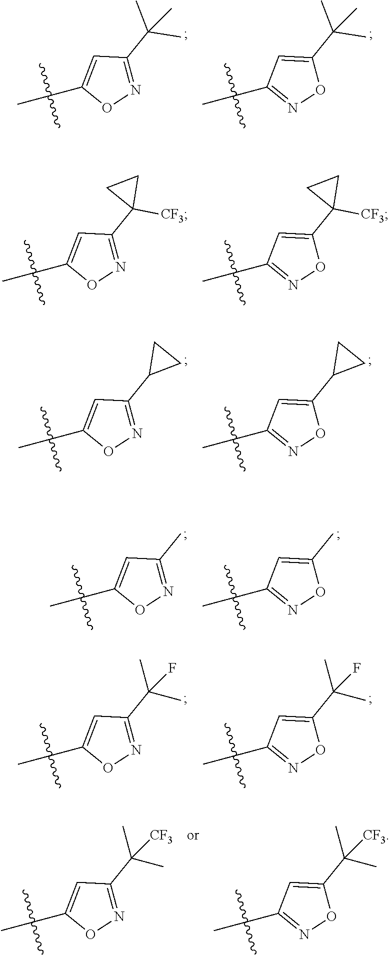

In other various embodiments, R^{4c} has one of the following structures:

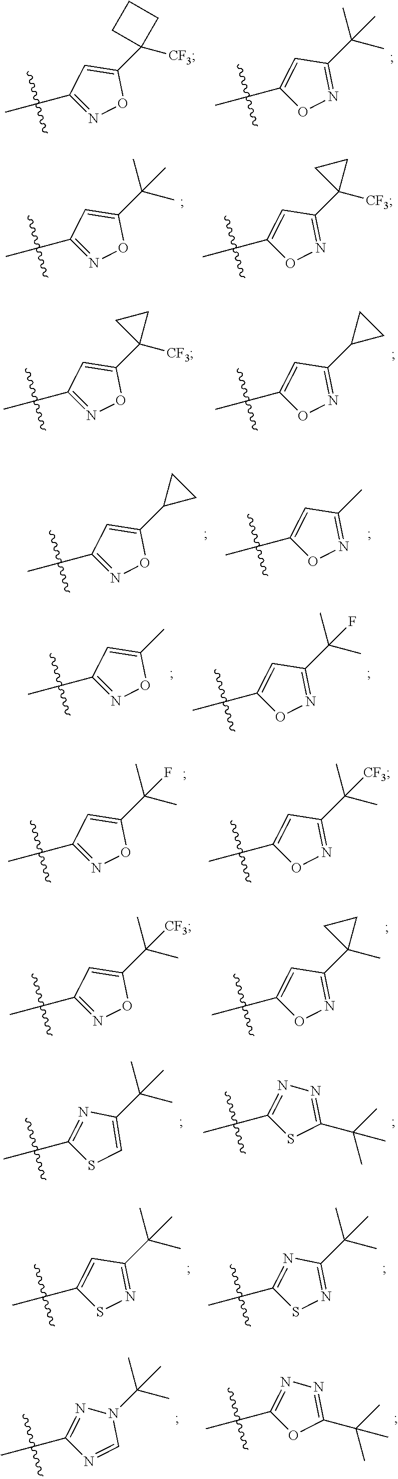

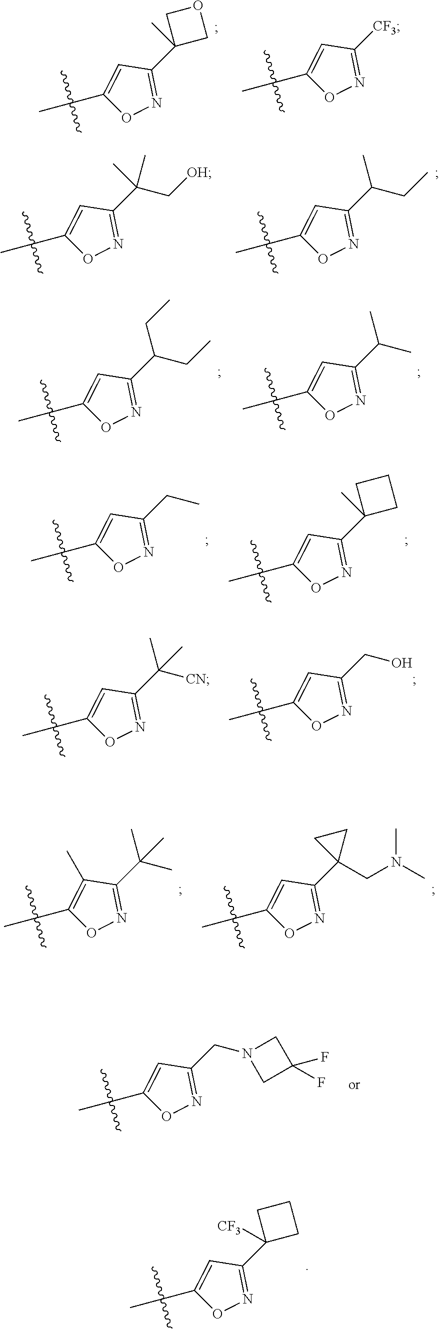

In other various embodiments, R^{4c} has one of the following structures:

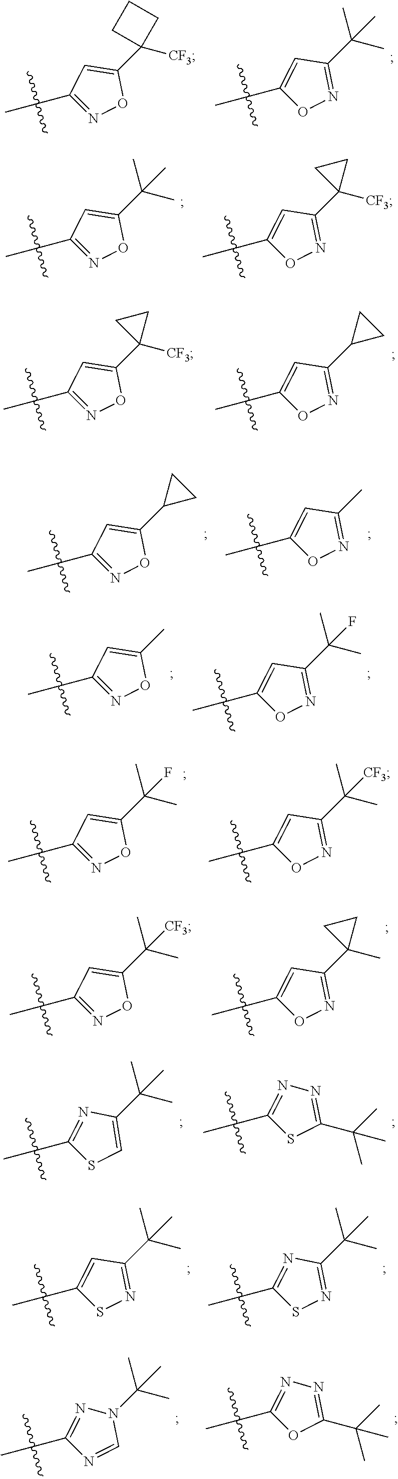

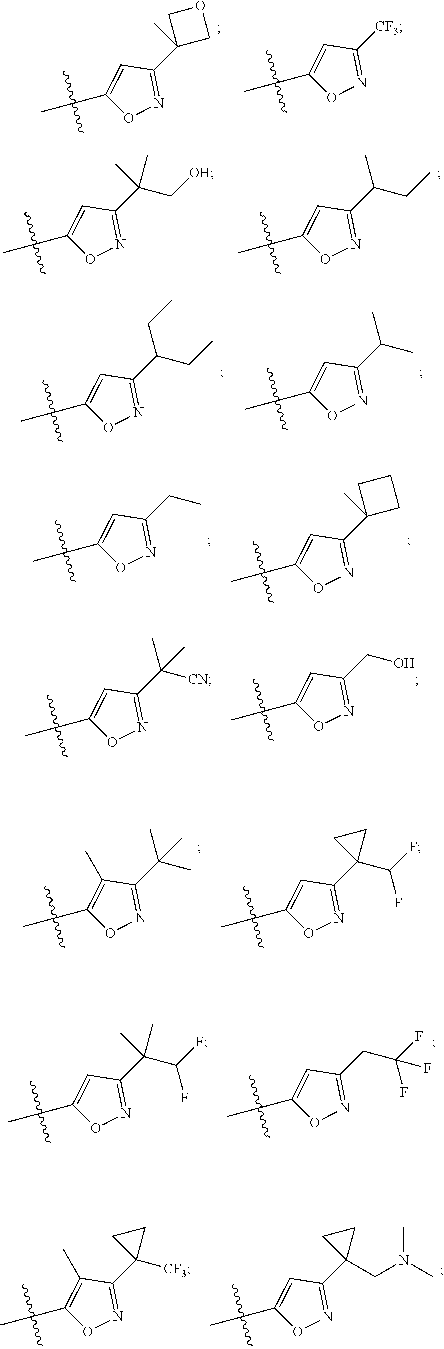

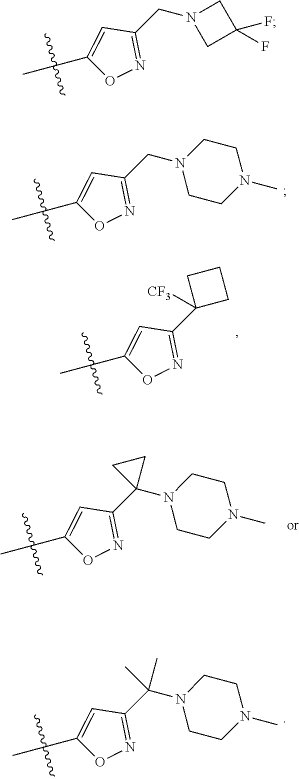 [SMILES: *c1cc(CN2CC(F)(F)C2)no1 ; *c1cc(CN2CCN(C)CC2)no1 ; *c1cc(C2(C(F)(F)F)CCC2)no1 ; *c1cc(C2(N3CCN(C)CC3)CC2)no1 ; *c1cc(C(C)(C)N2CCN(C)CC2)no1]

In certain specific embodiments. R^{2c} C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₃-C₈ alkoxy and 3-8 membered heterocyclyl, and

R^{4c} has one of the following structures:

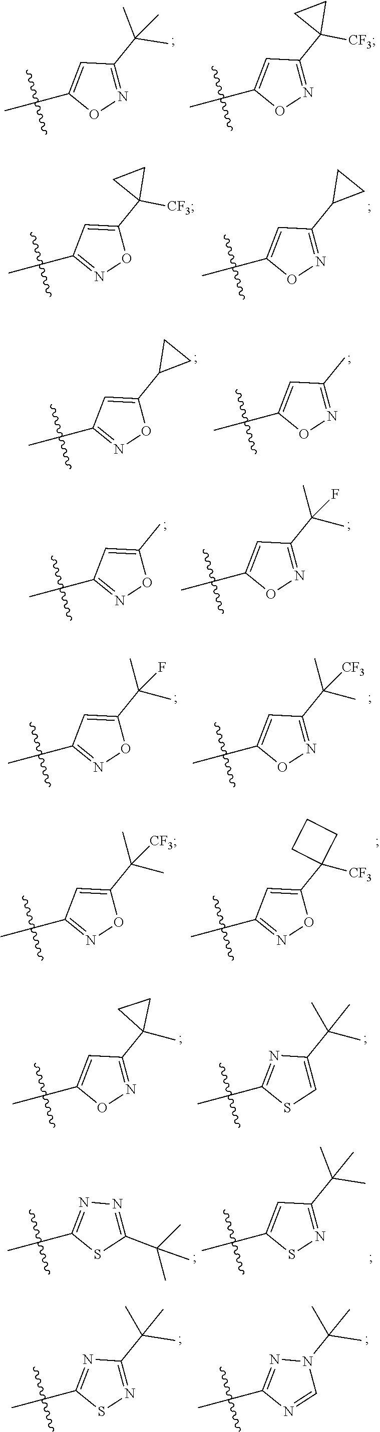

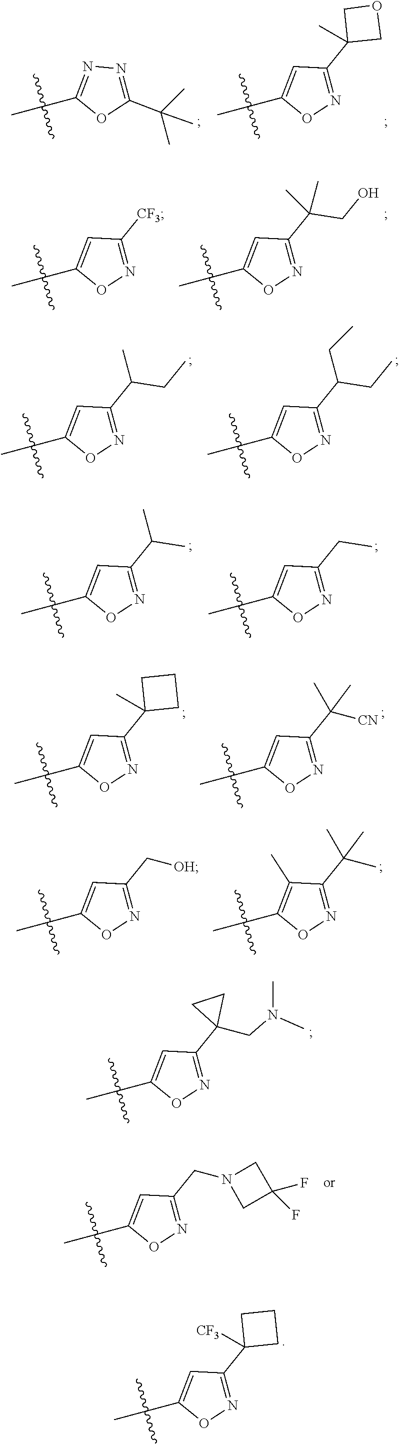

In certain specific embodiments, R^{2c} C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl; cyano, aminyl, C₁-C₆ alkyl, C₁-C₆ alkenyl, C₂-C₆ alkynyl, C₁-C₆ alkoxy and 3-8 membered heterocyclyl; and

R^{4c} has one of the following structures:

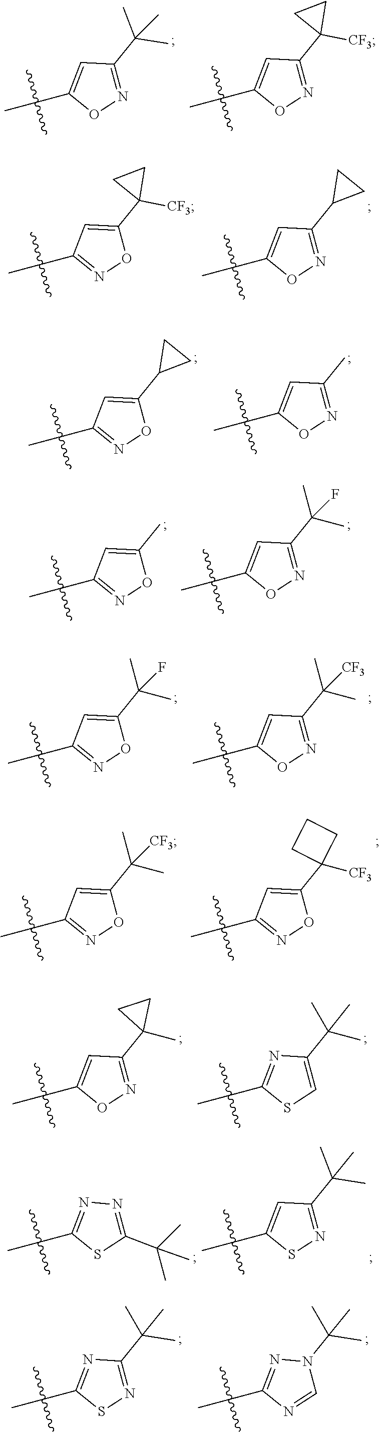

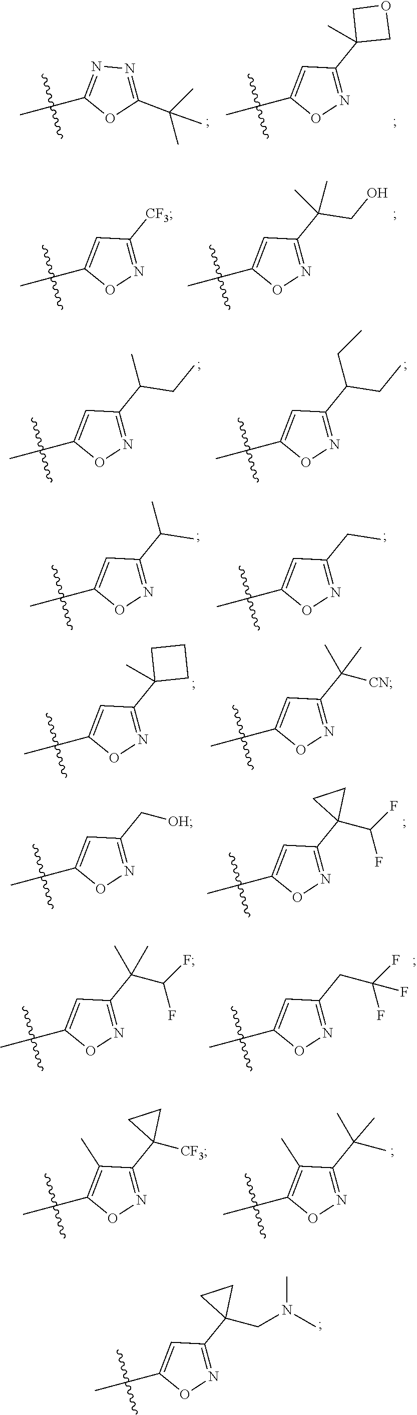

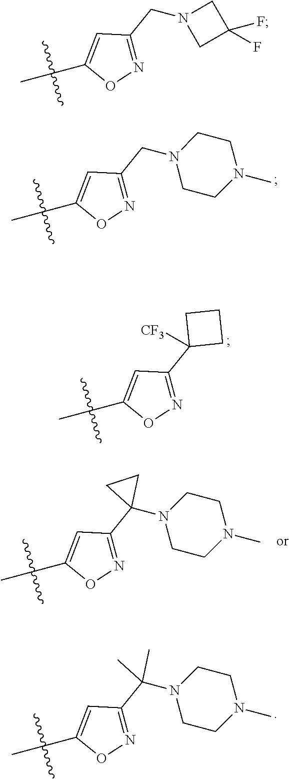 [SMILES: *c1cc(CN2CC(F)(F)C2)no1 ; *c1cc(CN2CCN(C)CC2)no1 ; *c1cc(C2(C(F)(F)F)CCC2)no1 ; *c1cc(C2(N3CCN(C)CC3)CC2)no1 ; *c1cc(C(C)(C)N2CCN(C)CC2)no1]

In more specific embodiments, R^{2c} is C₁-C₆ alkyl substituted with hydroxyl or C₁-C₆ alkoxy. In some embodiments, R^{2c} has one of the following structures:

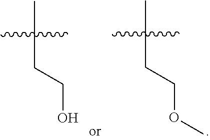

In other embodiments, R^{5c} is H. In other embodiments, R^{5c} C₁-C₆ alkyl, such as methyl.

In certain embodiments, Y^{c} is C(H)(OH). In other embodiments, Y^{c} is NH.

In various embodiments, A3 is C₆-C₁₀ aryl, C₃-C₁₀ cycloalkyl or 5-6 membered monocyclic heteroaryl, each of which is optionally substituted with one or more R^{6c}. It is understood that A3 is a divalent radical.

In certain embodiments, A3 is a divalent optionally substituted C₆₋₁₀ aryl. In certain embodiments, A3 is a divalent optionally substituted 3-8 membered saturated or partially unsaturated carbocyclic ring. In certain embodiments, A3 is a divalent optionally substituted 3-10 membered heterocylic ring having 1-4 heteroatoms independently selected from nitrogen, oxygen, or sulfur. In certain embodiments, A3 is a divalent optionally substituted 5-6 membered monocyclic heteroaryl ring having 1-4 heteroatoms independently selected from nitrogen, oxygen, or sulfur.

In certain embodiments, A3 is a divalent group selected from phenyl, pyridinyl, cyclohexyl, and cyclohexenyl; each of which is optionally substituted. In other embodiments, A3 is phenyl. In different embodiments, A3 is saturated or unsaturated cyclohexyl. In more embodiments, A3 is pyridinyl.

In certain embodiments, A3 is pyrimidinyl, which is optionally substituted.

In any of the foregoing embodiments, A3 is unsubstituted. In different of the foregoing embodiments, A3 is substituted with one or more R^{6c}. For example, in some embodiments R^{6c} is halo. In some embodiments, R^{6c} is chloro or fluoro. In other embodiments, R^{6c} is fluoro.

In some embodiments, R^{6c} is C₁-C₆ hydroxylalkyl. In some embodiments, C₁-C₆ hydroxylalkyl is —CH₂CH₂OH. In other embodiments, R^{6c} is cyano. In some embodiments, R^{6c} is C₁-C₆ alkoxy. In more specific embodiments, the C₁-C₆ alkoxy is methoxy.

In certain embodiments, A3 is a divalent group selected from phenyl, naphthyl, cyclopropyl, cyclobutyl, cyclopentyl, cyclohexyl, cyclohexenyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, dithiazinyl, tetrahydrofuranyl, furanyl, furazanyl, imidazolidinyl, imidazolinyl, imidazolyl, 11-indazolyl, indolenyl, indolinyl, indolizinyl, indolyl, 3-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2-pyrrolyl, pyrrolyl, quinazolinyl, quinolinyl, 4H-quinolizinyl, quinoxalinyl, quinuclidinyl, tetrahydro furanyl, tetrahydroisoquinolinyl, tetrahydroquinolinyl, thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, and xanthenyl; each of which is optionally substituted.

In specific embodiments, A3 has one of the following structures:

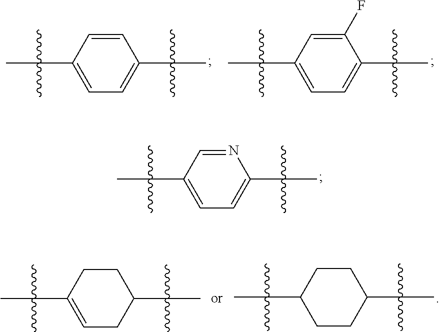 [SMILES: *c1ccc(*)cc1 ; *c1ccc(*)c(F)c1 ; *c1ccc(*)nc1 ; *C1=CCC(*)CC1 ; *C1CCC(*)CC1]

In some specific embodiments, A3 has one of the following structures:

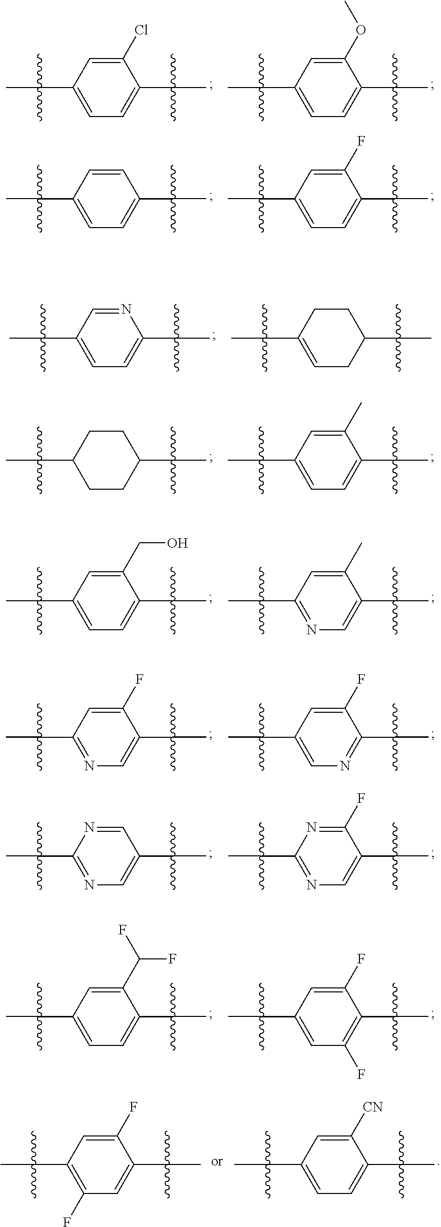

In certain embodiments, the compound has the following Structure (Ic-IA):

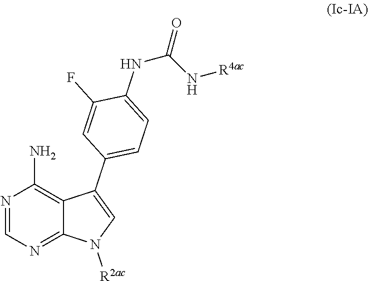

or a pharmaceutically acceptable salt, stereoisomer or prodrug thereof, wherein:

R^{2ac} is C₁-C₆ alkyl or C₃-C₈ cycloalkyl optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;

R^{4ac} is isoxazolyl optionally substituted with one more substituents selected from C₁-C₆ haloalkyl, C₃-C₈ cycloalkyl, or C₃-C₈ haloalkylcycloalkyl.

In more specific embodiments, R^{2ac} is a branched C₁-C₆ alkyl substituted with hydroxyl. In some embodiments, R^{2ac} is C₃-C₈ cycloalkyl. In more specific embodiments, R^{2ac} has one of the following structures:

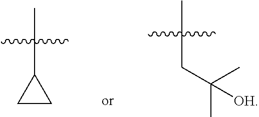

In certain embodiments, R^{4ac} is isoxazolyl substituted with C₃-C₈ haloalkylcycloalkyl. In some embodiments, R^{4ac} is C₃-C₈ fluoroalkylcycloalkyl. In still more specific embodiments, R^{4ac} is fluoroalkylcyclopropyl or fluoroalkylcyclobutyl. In more specific embodiments, R^{4ac} has one of the following structures:

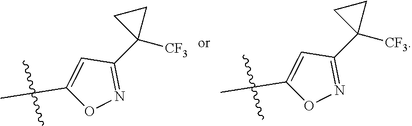 [SMILES: *c1cc(C2(C(F)(F)F)CC2)no1 ; *c1cc(C2(C(F)(F)F)CC2)no1]

In some embodiments, X^{x} is CH. In some more specific embodiments, the compound has the following Structure (Ic-IB):

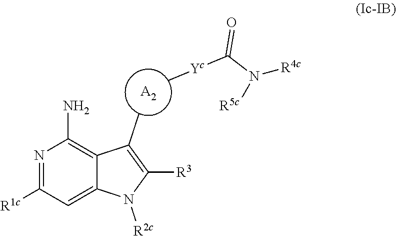

or a pharmaceutically acceptable salt, stereoisomer or prodrug thereof, wherein:

A2 is C₆-C₁₀ aryl, C₃-C₁₀ cycloalkyl, 3-10 membered heterocyclyl or 5-6 membered monocyclic heteroaryl, each of which is optionally substituted with one or more R^{6c};

X^{c} is CH or N;

Y^{c} is CHOH or NH;

R^{1c} is H or C₁-C₆ alkyl;

R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₁-C₆ alkenyl, C₂-C₆ alkynyl and C₁-C₆ alkoxy;

R^{4c} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl;

R^{5c} is H, C₁-C₆ alkyl, C₂-C₆ alkenyl, C₂-C₆ alkynyl, C₃-C₈ cycloalkyl, 3- to 8-membered heterocyclyl, C₁-C₆ to aryl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl and C₁-C₆ alkoxy; and

R^{6c} is, at each occurrence, independently halo, C₁-C₆ alkyl, C₁-C₆ alkoxy, cyano, C₁-C₆ hydroxylalkyl or C₁-C₆ haloalkyl.

In a certain embodiment, the compound of Structure (Ic) is a modulator of the NLRP3 inflammasome.

In a specific embodiment, the compound of Structure (Ic) is an inhibitor of NEK7 in a patient or in a biological sample.

In various different embodiments, the compound has one of the structures set forth in Table 1c below, or a pharmaceutically acceptable salt, stereoisomer or prodrug thereof. Compounds in Table 1c were prepared as described in the Examples or methods known in the art and analyzed by mass spectrometry and/or ¹H NMR.

TABLE 1c
Representative Compounds of Structure (Ic)

| No. | Structure | Name |
|---|---|---|
| Ic-1 | 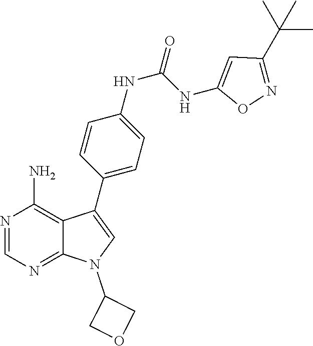 [SMILES: CC(C)(C)c1cc(NC(=O)Nc2ccc(-c3cn(C4COC4)c4ncnc(N)c34)cc2)on1] | 1-(4-(4-amino-7- (oxetan-3-yl)-7H- pyrrolo[2,3- d]pyrimidin-5- yl)phenyl)-3-(3-(tert- butyl)isoxazol-5- yl)urea |
| Ic-2 | 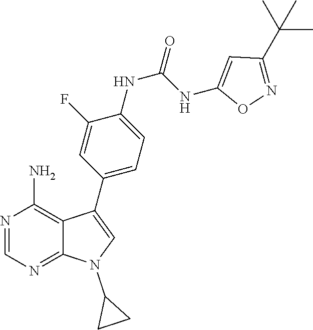 [SMILES: CC(C)(C)c1cc(NC(=O)Nc2ccc(-c3cn(C4CC4)c4ncnc(N)c34)cc2F)on1] | 1-(4-(4-amino-7- cycloplopyl-7H- pyrrolo[2,3- d]pyrimidin-5-yl)-2- fluorophenyl)-3-(3- (tert-butyl)isoxazol-5- yl)urea |
| Ic-3 | 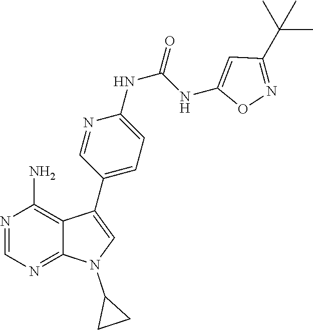 [SMILES: CC(C)(C)c1cc(NC(=O)Nc2ccc(-c3cn(C4CC4)c4ncnc(N)c34)cn2)on1] | 1-(5-(4-amino-7- cyclopropyl-7H- pyrrolo[2,3- d]pyrimidin-5- yl)pyridin-2-yl)-3-(3- (tert-butyl)isoxazol-5- yl)urea |
| Ic-4 | 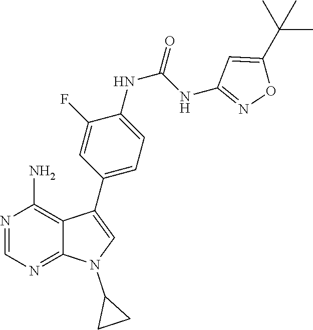 [SMILES: CC(C)(C)c1cc(NC(=O)Nc2ccc(-c3cn(C4CC4)c4ncnc(N)c34)cc2F)no1] | 1-(4-(4-amino-7- cyclopropyl-7H- pyrrolo[2,3- d]pyrimidin-5-yl)-2- fluorophenyl)-3-(5- (tert-butyl)isoxazol-3- yl)urea |
| Ic-5 | 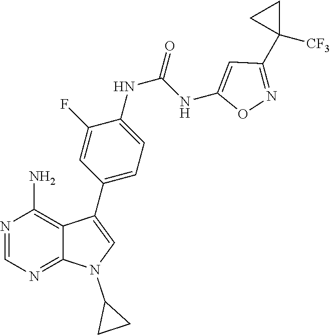 [SMILES: Nc1ncnc2c1c(-c1ccc(NC(=O)Nc3cc(C4(C(F)(F)F)CC4)no3)c(F)c1)cn2C1CC1] | 1-(4-(4-amino-7- cyclopropyl-7H- pynolo[2,3- d]pyrimidin-5-yl)-2- fluorophenyl)-3-(3-(1- (trifluoromethyl) cyclopropyl) isoxazol-5- yl)urea |
| Ic-6 | 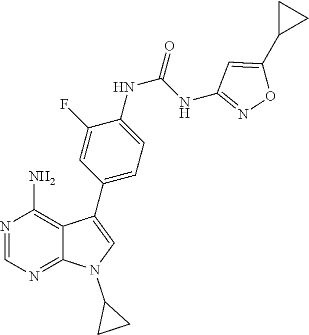 [SMILES: Nc1ncnc2c1c(-c1ccc(NC(=O)Nc3cc(C4CC4)on3)c(F)c1)cn2C1CC1] | 1-(4-(4-amino-7- cyclopropyl-7H- pyrrolo[2,3- d]pyrimidin-5-yl)-2- fluorophenyl)-3-(5- cyclopropylisoxazol-3- yl)urea |
| Ic-7 | 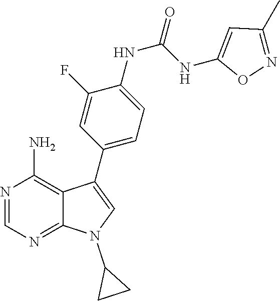 [SMILES: Cc1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3- methylisoxazol-5- yl)urea |
| Ic-8 | 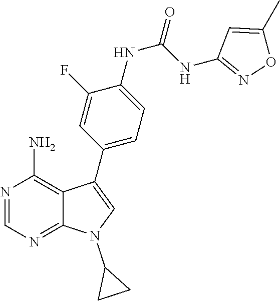 [SMILES: Cc1cc(NC(=O)Nc2ccc(-c3cn(C4CC4)c4ncnc(N)c34)cc2F)no1] | 1-(4-(4-amino-7- cyclopropyl-7H- pyrrolo[2,3- d]pyrimidin-5-yl)-2- fluorophenyl)-3-(5- methylisoxazol-3- yl)urea |
| Ic-9 | 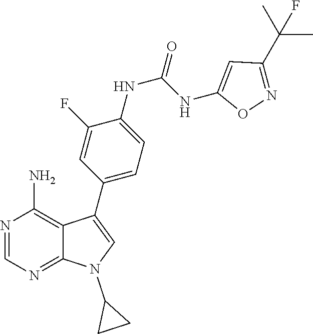 [SMILES: CC(C)(F)c1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3-(2- fluoropropan-2- yl)isoxazol-5-yl)urea |
| Ic-10 | 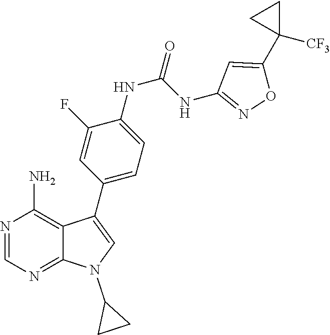 [SMILES: Nc1ncnc2c1c(-c1ccc(NC(=O)Nc3cc(C4(C(F)(F)F)CC4)on3)c(F)c1)cn2C1CC1] | 1-(4-(4-amino-7- cyclopropyl-7H- pyrrolo[2,3- d]pyrimidin-5-yl)-2- fluorophenyl)-3-(5-(1- (trifluoromethyl) cyclopropyl) isoxazol-3-yl)urea |
| Ic-11 | 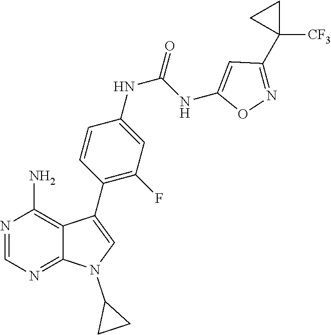 [SMILES: Nc1ncnc2c1c(-c1ccc(NC(=O)Nc3cc(C4(C(F)(F)F)CC4)no3)cc1F)cn2C1CC1] | 1-(4-(4-amino-7- cyclopropyl-7H- pyrrolo[2,3- d]pyrimidin-5-yl)-3- fluorophenyl)-3-(3-(1- (trifluoromethyl) cyclopropyl) isoxazol-5-yl)urea |
| Ic-12 | 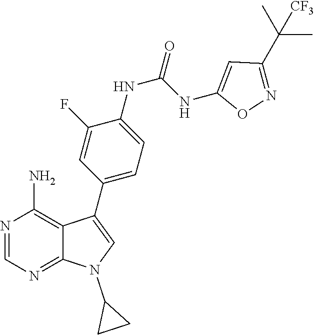 [SMILES: CC(C)(c1cc(NC(=O)Nc2ccc(-c3cn(C4CC4)c4ncnc(N)c34)cc2F)on1)C(F)(F)F] | 1-(4-(4-amino-7- cyclopropyl-7H- pyrrolo[2,3- d]pyrimidin-5-yl)-2- fluorophenyl)-3-(3- (1,1,1-trifluoro-2- methylpropan-2- yl)isoxazol-5-yl)urea |
| Ic-13 | 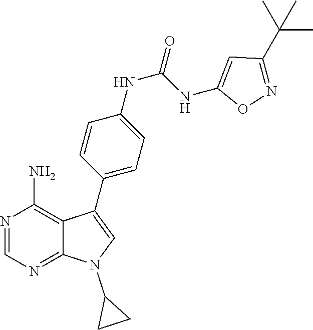 [SMILES: CC(C)(C)c1cc(NC(=O)Nc2ccc(-c3cn(C4CC4)c4ncnc(N)c34)cc2)on1] | 1-(4-(4-amino7 cyclopropy-7H- pyrrolo[2,3- d]pyrimidin-5- yl)phenyl)-3-(3-(tert- butyl)isoxazol-5- yl)urea |
| Ic-14 | 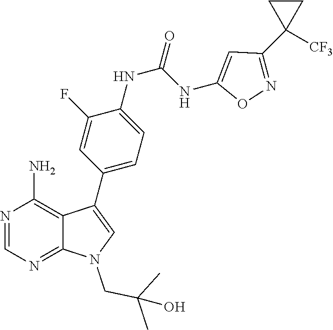 [SMILES: CC(C)(O)Cn1cc(-c2ccc(NC(=O)Nc3cc(C4(C(F)(F)F)CC4)no3)c(F)c2)c2c(N)ncnc21] | 1-(4-(4-amino-7-(2- hydroxy-2- methylpropyl)-7H- pyrrolo[2,3- d]pyrimidin-5-yl)-2- fluorophenyl)-3-(3-(1- (trifluoromethyl) cyclopropyl) isoxazol-5-yl)urea |
| Ic-15 | 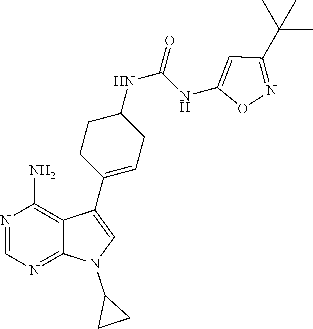 [SMILES: CC(C)(C)c1cc(NC(=O)NC2CC=C(c3cn(C4CC4)c4ncnc(N)c34)CC2)on1] | 1-(4-(4-amino-7- cyclopropyl-7H- pyrrolo[2,3- d]pyrimidin-5- yl)cyclohex-3-en-1-yl)- 3-(3-(tert- butyl)isoxazol-5- yl)urea |
| Ic-16 | 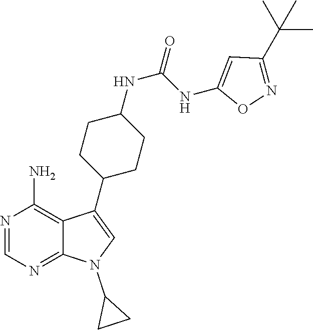 [SMILES: CC(C)(C)c1cc(NC(=O)NC2CCC(c3cn(C4CC4)c4ncnc(N)c34)CC2)on1] | 1-(4-(4-amino-7- cyclopropyl-7H- pyrrolo[2,3- d]pyrimidin-5- yl)cyclohexyl)-3-(3- (tert-butyl)isoxazol-5- yl)urea |
| Ic-17 | 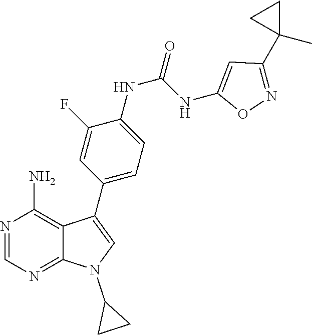 [SMILES: CC1(c2cc(NC(=O)Nc3ccc(-c4cn(C5CC5)c5ncnc(N)c45)cc3F)on2)CC1] | 1-(4-(4-amino-7- cyclopropyl-7H- pyrrolo[2,3- d]pyrimidin-5-yl)-2- fluorophenyl)-3-(3-(1- methylcyclopropyl) isoxazol-5-yl)urea |
| Ic-18 | 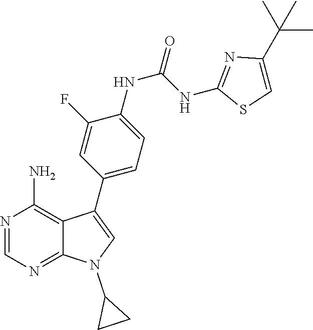 [SMILES: CC(C)(C)c1csc(NC(=O)Nc2ccc(-c3cn(C4CC4)c4ncnc(N)c34)cc2F)n1] | 1-(4-(4-amino-7- cyclopropyl-7H- pyrrolo[2,3- d]pyrimidin-5-yl)-2- fluorophenyl)-3-(-4- (tert-butyl)thiazol-2- yl)urea |
| Ic-19 | 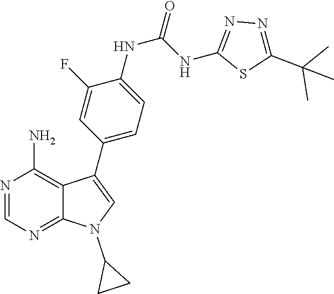 [SMILES: CC(C)(C)c1nnc(NC(=O)Nc2ccc(-c3cn(C4CC4)c4ncnc(N)c34)cc2F)s1] | 1-(4-(4-amino-7- cyclopropyl-7H- pyrrolo[2,3- d]pyrimidin-5-yl)-2- fluorophenyl)-3-(5- (tert-butyl)-1,3,4- thiadiazol-2-yl)urea |
| Ic-20 | 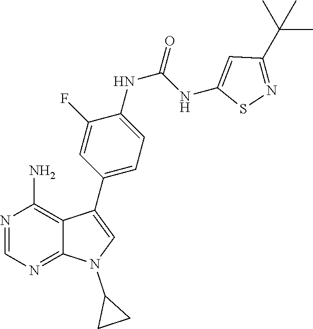 [SMILES: CC(C)(C)c1cc(NC(=O)Nc2ccc(-c3cn(C4CC4)c4ncnc(N)c34)cc2F)sn1] | 1-(4-(4-amino-7- cyclopropyl-7H- pyrrolo[2,3- d]pyrimidin-5-yl)-2- fluorophenyl)-3-(3- (tert-butyl)isothiazol-5- yl)urea |
| Ic-21 | 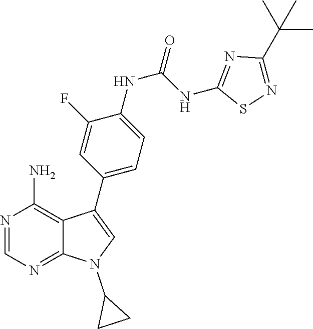 [SMILES: CC(C)(C)c1nsc(NC(=O)Nc2ccc(-c3cn(C4CC4)c4ncnc(N)c34)cc2F)n1] | 1-(4-(4-amino-7 cyclopropyl-7H- pyrrolo[2,3- d]pyrimidin-5-yl)-2- fluorophenyl)-3-(3- (tert-butyl)-1,2,4- thiadiazol-5-yl)urea |
| Ic-22 | 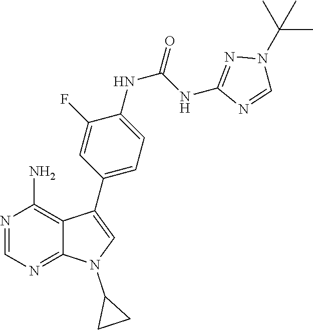 [SMILES: CC(C)(C)n1cnc(NC(=O)Nc2ccc(-c3cn(C4CC4)c4ncnc(N)c34)cc2F)n1] | 1-(4-(4-amino-7- cyclopropyl-7H- pyrrolo[2,3- d]pyrimidin-5-yl)-2- fluorophenyl)-3-(3- (tert-butyl)-1,2,4 thiadiazol-5-yl)urea |
| Ic-23 | 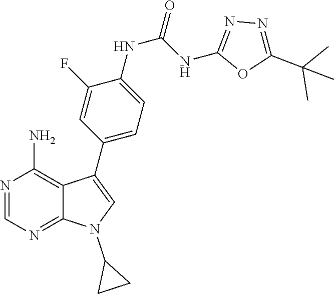 [SMILES: CC(C)(C)c1nnc(NC(=O)Nc2ccc(-c3cn(C4CC4)c4ncnc(N)c34)cc2F)o1] | 1-(4-(4-amino-7- cyclopropyl-7H- pyrrolo[2,3- d]pyrimidin-5-yl)-2- fluorophenyl)-3-(5- (tert-butyl)-1,3,4- oxadiazol-2-yl)urea |
| Ic-24 | 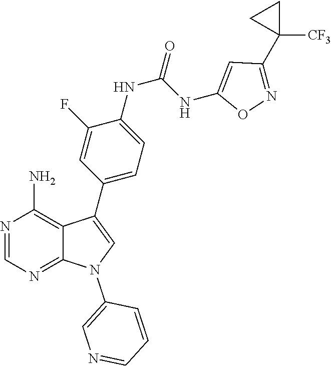 [SMILES: Nc1ncnc2c1c(-c1ccc(NC(=O)Nc3cc(C4(C(F)(F)F)CC4)no3)c(F)c1)cn2-c1cccnc1] | 1-(4-(4-amino-7- (pyridin-3-yl)-7H- pyrrolo[2,3- d]pyrimidin-5-yl)-2- fluorophenyl)-3-(3-(1- (trifluoromethyl) cyclopropyl) isoxazol-5-yl)urea |
| Ic-25 | 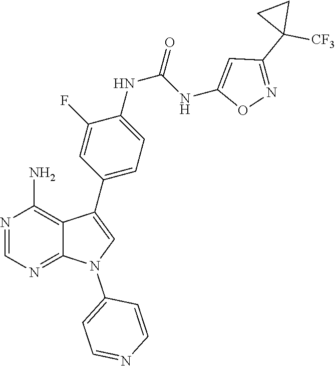 [SMILES: Nc1ncnc2c1c(-c1ccc(NC(=O)Nc3cc(C4(C(F)(F)F)CC4)no3)c(F)c1)cn2-c1ccncc1] | 1-(4-(4-amino-7- (pyridin-4-yl)-7H- pyrrolo[2,3- d]pyrimidin-5-yl)-2- fluorophenyl)-3-(3-(1- (trifluoromethyl) cyclopropyl) isoxazol-5-yl)urea |
| Ic-26 | 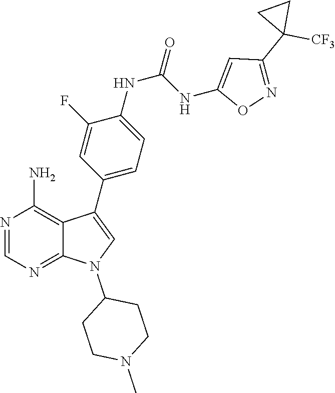 [SMILES: CN1CCC(n2cc(-c3ccc(NC(=O)Nc4cc(C5(C(F)(F)F)CC5)no4)c(F)c3)c3c(N)ncnc32)CC1] | 1-(4-(4-amino-7-(1- methylpiperidin-4-yl)- 7H-pyrrolo[2,3- d]pyrimidin-5-yl)-2- fluorophenyl)-3-(3-(1- (trifluoromethyl) cyclopropyl) isoxazol-5-yl)urea |
| Ic-27 | 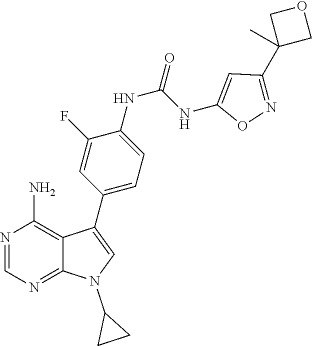 [SMILES: CC1(c2cc(NC(=O)Nc3ccc(-c4cn(C5CC5)c5ncnc(N)c45)cc3F)on2)COC1] | 1-(4-(4-amino-7- cyclopropyl-7H- pyrrolo[2,3- d]pyrimidin-5-yl)-2- fluorophenyl)-3-(3-(3- methyloxetan-3- yl)isoxazol-5-yl)urea |
| Ic-28 | 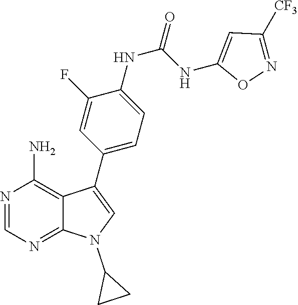 [SMILES: Nc1ncnc2c1c(-c1ccc(NC(=O)Nc3cc(C(F)(F)F)no3)c(F)c1)cn2C1CC1] | 1-(4-(4-amino-7- cyclopropyl-7H- pyrrolo[2,3- d]pyrimidin-5-yl)-2- fluorophenyl)-3-(3- (trifluoromethyl) isoxazol-5-yl)urea |
| Ic-29 | 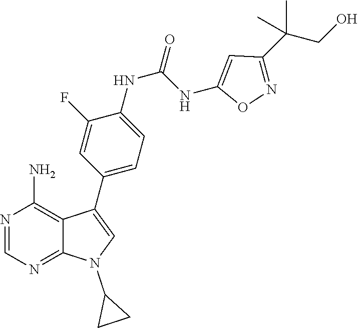 [SMILES: CC(C)(CO)c1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3-(1- hydroxy-2- methylpropan-2- yl)isoxazol-5-yl)urea |
| Ic-30 | 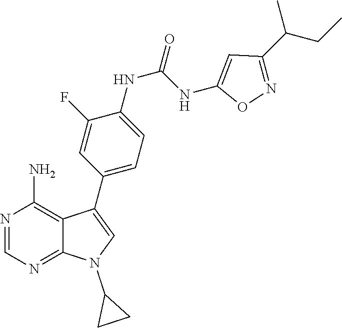 [SMILES: CCC(C)c1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3- (sec-butyl)isoxazol-5- yl)urea |
| Ic-31 | 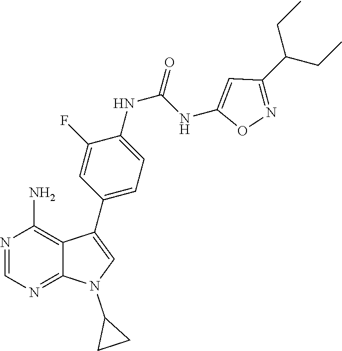 [SMILES: CCC(CC)c1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3- (pentan-3-yl)isoxazol- 5-yl)urea |
| Ic-32 | 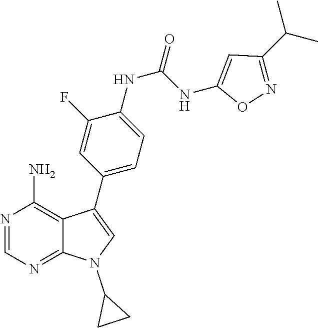 [SMILES: CC(C)c1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3- isopropylisoxazol-5- yl)urea |
| Ic-33 | 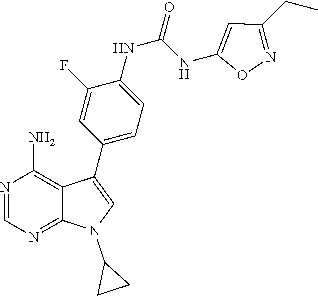 [SMILES: CCc1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3- ethylisoxazol-5-yl)urea |
| Ic-34 | 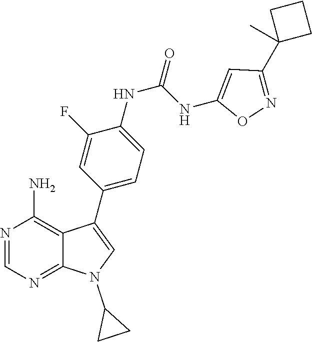 [SMILES: CC1(c2cc(NC(=O)Nc3ccc(-c4cn(C5CC5)c5ncnc(N)c45)cc3F)on2)CCC1] | 1-(4-(4-amino-7- cyclopropyl-7H- pyrrolo[2,3- d]pyrimidin-5-yl)-2- fluorophenyl)-3-(3-(1- methylcyclobutyl) isoxazol-5-yl)urea |
| Ic-35 | 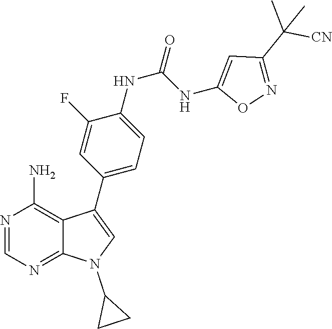 [SMILES: CC(C)(C#N)c1cc(NC(=O)Nc2ccc(-c3cn(C4CC4)c4ncnc(N)c34)cc2F)on1] | 1-(4-(4-amino-7- cyclopropyl-7H- pyrrolo[2,3- d]pyrimidin-5-yl)-2- fluorophenyl)-3-(3-(2- cyanopropan-2- yl)isoxazol-5-yl)urea |
| Ic-36 | 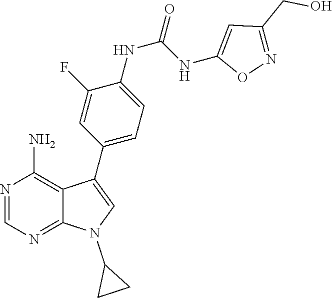 [SMILES: Nc1ncnc2c1c(-c1ccc(NC(=O)Nc3cc(CO)no3)c(F)c1)cn2C1CC1] | 1-(4-(4-amino-7- cyclopropyl-7H- pyrrolo[2,3- d]pyrimidin-5-yl)-2- fluorophenyl)-3-(3- (hydroxymethyl) isoxazol-5-yl)urea |
| Ic-37 | 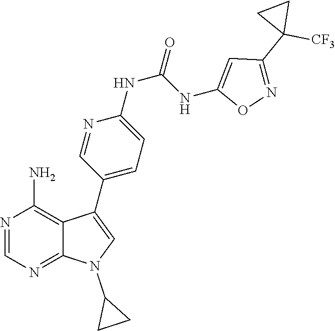 [SMILES: Nc1ncnc2c1c(-c1ccc(NC(=O)Nc3cc(C4(C(F)(F)F)CC4)no3)nc1)cn2C1CC1] | 1-(5-(4-amino-7- cyclopropyl-7H- pyrrolo[2,3- d]pyrimidin-5- yl)pyridin-2-yl)-3-(3- (1-(trifluoromethyl) cyclopropyl) isoxazol-5-yl)urea |
| Ic-38 | 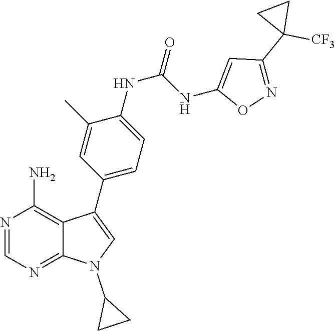 [SMILES: Cc1cc(-c2cn(C3CC3)c3ncnc(N)c23)ccc1NC(=O)Nc1cc(C2(C(F)(F)F)CC2)no1] | 1-(4-(4-amino-7- cyclopropyl-7H- pyrrolo[2,3- d]pyrimidin-5-yl)-2- methylphenyl)-3-(3-(1- (trifluoromethyl) cyclopropyl) isoxazol-5-yl)urea |
| Ic-39 | 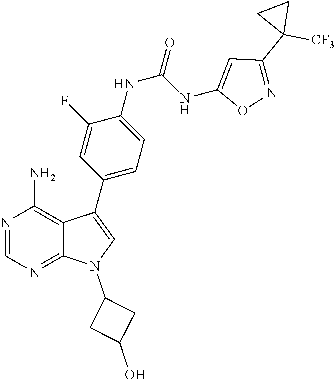 [SMILES: Nc1ncnc2c1c(-c1ccc(NC(=O)Nc3cc(C4(C(F)(F)F)CC4)no3)c(F)c1)cn2C1CC(O)C1] | 1-(4-(4-amino-7-(3- hydroxycyclobutyl)- 7H-pyrrolo[2,3- d]pyrimidin-5-yl)-2- fluorophenyl)-3-(3-(1- (trifluoromethyl) cyclopropyl) isoxazol-5-yl)urea |
| Ic-40 | 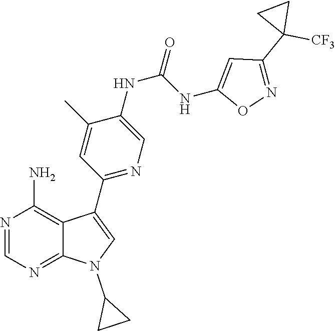 [SMILES: Cc1cc(-c2cn(C3CC3)c3ncnc(N)c23)ncc1NC(=O)Nc1cc(C2(C(F)(F)F)CC2)no1] | 1-(6-(4-amino-7- cyclopropyl-7H- pyrrolo[2,3- d]pyrimidin-5-yl)-4- methylpyridin-3-yl)-3- (3-(1-(trifluoromethyl) cyclopropyl) isoxazol-5-yl)urea |
| Ic-41 | 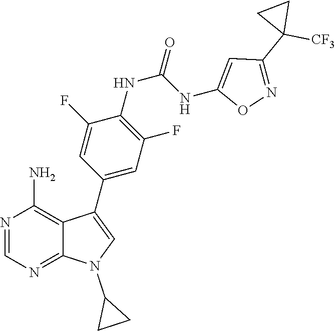 [SMILES: Nc1ncnc2c1c(-c1cc(F)c(NC(=O)Nc3cc(C4(C(F)(F)F)CC4)no3)c(F)c1)cn2C1CC1] | 1-(4-(4-amino-7- cyclopropyl-7H pyrrolo[2,3- d]pyrimidin-5-yl)-2,6- difluorophenyl)-3-(3- (1-(trifluoromethyl) cyclopropyl) isoxazol-5-yl)urea |
| Ic-42 | 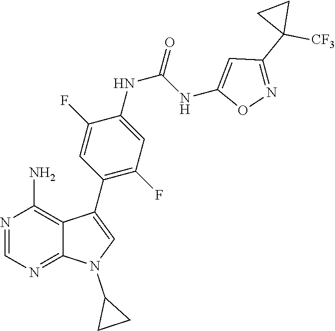 [SMILES: Nc1ncnc2c1c(-c1cc(F)c(NC(=O)Nc3cc(C4(C(F)(F)F)CC4)no3)cc1F)cn2C1CC1] | 1-(4-(4-amino-7- oyclopropyl-7H- pyrrolo[2,3- d]pyrimidin-5-yl)2,5- difluorophenyl)-3-(3- (1-(trifluoromethyl) cyclopropyl) isoxazol-5-yl)urea |
| Ic-43 | 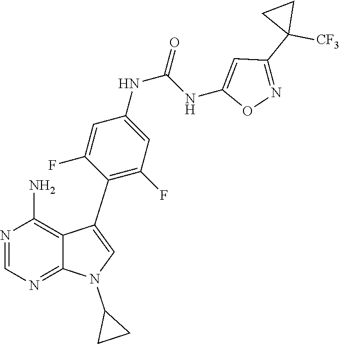 [SMILES: Nc1ncnc2c1c(-c1c(F)cc(NC(=O)Nc3cc(C4(C(F)(F)F)CC4)no3)cc1F)cn2C1CC1] | 1-(4-(4-amino-7- cyclopropyl-7H- pyrrolo[2,3- d]pyrimidin-5-yl)-3,5- difluorophenyl)-3-(3- (1-(trifluoromethyl) cyclopropyl) isoxazol-5-yl)urea |
| Ic-44 | 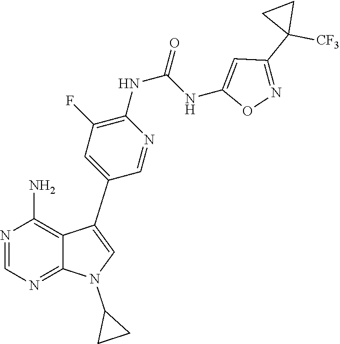 [SMILES: Nc1ncnc2c1c(-c1cnc(NC(=O)Nc3cc(C4(C(F)(F)F)CC4)no3)c(F)c1)cn2C1CC1] | 1-(5-(4-amino-7- cyclopropyl-7H- pyrrolo[2,3- d]pyrimidin-5-yl)-3- fluoropyridin-2-yl)-3- (3-(1-(trifluoromethyl) cyclopropyl) isoxazol-5-yl)urea |
| Ic-45 | 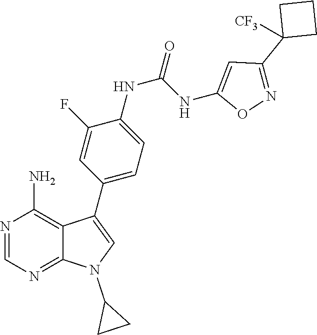 [SMILES: Nc1ncnc2c1c(-c1ccc(NC(=O)Nc3cc(C4(C(F)(F)F)CCC4)no3)c(F)c1)cn2C1CC1] | 1-(4-(4-amino-7- cyclopropyl-7H- pyrrolo[2,3- d]pyrimidin-5-yl)-2- fluorophenyl)-3-(3-(1- (trifluoromethyl) cyclobutyl)isoxazol- 5-yl)urea |
| Ic-46 | 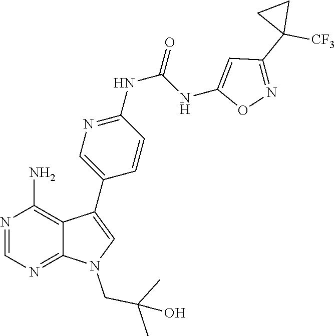 [SMILES: CC(C)(O)Cn1cc(-c2ccc(NC(=O)Nc3cc(C4(C(F)(F)F)CC4)no3)nc2)c2c(N)ncnc21] | 1-(5-(4-amino-7-(2- hydroxy-2- methylpropyl)-7H- pyrrolo[2,3- d]pyrimidin-5- yl)pyridin-2-yl)-3-(3- (1-(trifluoromethyl) cyclopropyl)isoxazol- 5-yl)urea |
| Ic-47 | 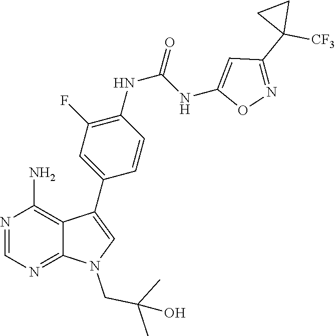 [SMILES: CC(C)(O)Cn1cc(-c2ccc(NC(=O)Nc3cc(C4(C(F)(F)F)CC4)no3)c(F)c2)c2c(N)ncnc21] | 1-(4-(4-amino-7-(2- hydroxy-2- methylpropyl)-7H- pyrrolo[2,3- d]pyrimidin-5-yl)-2- fluorophenyl)-3-(3-(1- (trifluoromethyl) cyclopropyl)isoxazol- 5-yl)urea |
| Ic-48 | 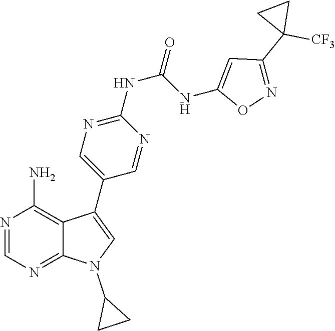 [SMILES: Nc1ncnc2c1c(-c1cnc(NC(=O)Nc3cc(C4(C(F)(F)F)CC4)no3)nc1)cn2C1CC1] | 1-(5-(4-amino-7- cyclopropyl-7H- pyrrolo[2,3- d]pyrimidin-5- yl)pyrimidin-2-yl)-3-(3- (1-(trifluoromethyl) cyclopropyl)isoxazol- 5-yl)urea |
| Ic-49 | 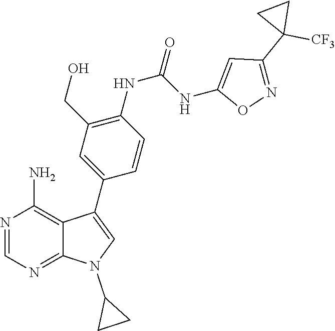 [SMILES: Nc1ncnc2c1c(-c1ccc(NC(=O)Nc3cc(C4(C(F)(F)F)CC4)no3)c(CO)c1)cn2C1CC1] | 1-(4-(4-amino-7- cyclopropyl-7H- pyrrolo[2,3- d]pyrimidin-5-yl)-2- (hydroxymethylphenyl)- 3-(3-(1- (trifluoromethyl) cyclopropyl)isoxazol- 5-yl)urea |
| Ic-50 | 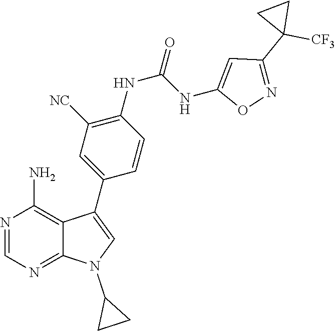 [SMILES: N#Cc1cc(-c2cn(C3CC3)c3ncnc(N)c23)ccc1NC(=O)Nc1cc(C2(C(F)(F)F)CC2)no1] | 1-(4-(4-amino-7- cyclopropyl-7H- pyrrolo[2,3- d]pyrimidin-5-yl)-2- cyanophenyl)-3-(3-(1- (trifluoromethyl) cyclopropyl)isoxazol- 5-yl)urea |
| Ic-51 | 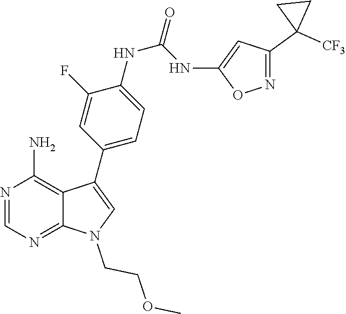 [SMILES: COCCn1cc(-c2ccc(NC(=O)Nc3cc(C4(C(F)(F)F)CC4)no3)c(F)c2)c2c(N)ncnc21] | 1-(4-(4-amino-7-(2- methoxyethyl)-7H- pyrrolo[2,3- d]pyrimidin-5-yl)-2- fluorophenyl)-3-(3-(1- (trifluoromethyl) cyclopropyl)isoxazol- 5-yl)urea |
| Ic-52 | 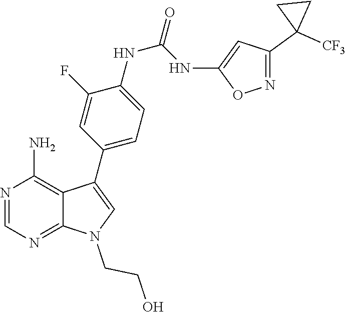 [SMILES: Nc1ncnc2c1c(-c1ccc(NC(=O)Nc3cc(C4(C(F)(F)F)CC4)no3)c(F)c1)cn2CCO] | 1-(4-(4-amino-7-(2- hydroxyethyl)-7H- pyrrolo[2,3- d]pyrimidin-5-yl)-2- fluorophenyl)-3-(3-(1- (trifluoromethyl) cyclopropyl) isoxazol-5-yl)urea |
| Ic-53 | 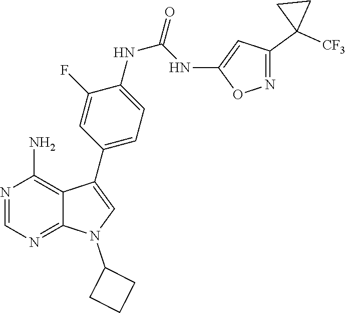 [SMILES: Nc1ncnc2c1c(-c1ccc(NC(=O)Nc3cc(C4(C(F)(F)F)CC4)no3)c(F)c1)cn2C1CCC1] | 1-(4-(4-amino-7- cyclobutyl-7H- pyrrolo[2,3- d]pyrimidin-5-yl)-2- fluorophenyl)-3-(3-(1- (trifluoromethyl) cyclopropyl) isoxazol-5-yl)urea |
| Ic-54 | 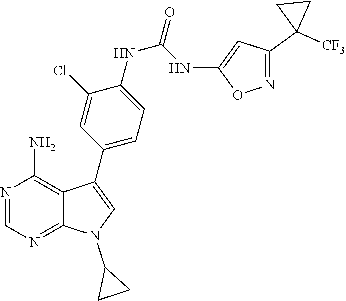 [SMILES: Nc1ncnc2c1c(-c1ccc(NC(=O)Nc3cc(C4(C(F)(F)F)CC4)no3)c(Cl)c1)cn2C1CC1] | 1-(4-(4-amino-7- cyclopropyl-7H- pyrrolo[2,3- d]pyrimidin-5-yl)-2- chlorophenyl)-3-(3-(1- (trifluoromethyl) cyclopropyl)isoxazol- 5-yl)urea |
| Ic-55 | 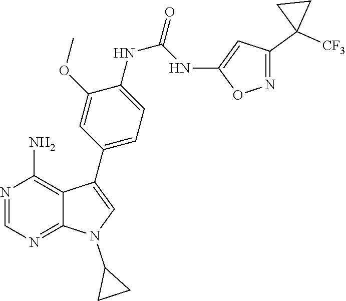 [SMILES: COc1cc(-c2cn(C3CC3)c3ncnc(N)c23)ccc1NC(=O)Nc1cc(C2(C(F)(F)F)CC2)no1] | 1-(4-(4-amino-7- cyclopropyl-7H- pyrrolo[2,3- d]pyrimidin-5-yl)-2- methoxyphenyl)-3-(3- (1-(trifluoromethyl) cyclopropyl)isoxazol- 5-yl)urea |
| Ic-56 | 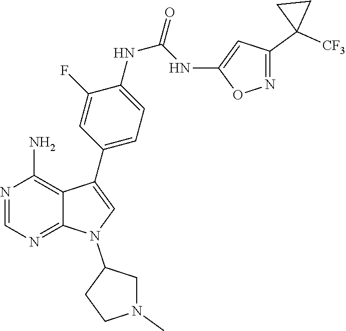 [SMILES: CN1CCC(n2cc(-c3ccc(NC(=O)Nc4cc(C5(C(F)(F)F)CC5)no4)c(F)c3)c3c(N)ncnc32)C1] | 1-(4-(4-amino-7-(1- methylpyrrolidin-3-yl)- 7H-pyrrolo[2,3- d]pyrimidin-5-yl)-2- fluorophenyl)-3-(3-(1- (trifluoromethyl) cyclopropyl)isoxazol- 5-yl)urea |
| Ic-57 | 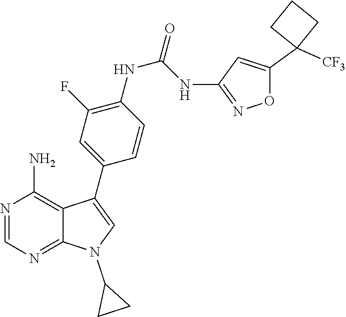 [SMILES: Nc1ncnc2c1c(-c1ccc(NC(=O)Nc3cc(C4(C(F)(F)F)CCC4)on3)c(F)c1)cn2C1CC1] | 1-(4-(4-amino-7- cyclopropyl-7H- pyrrolo[2,3- d]pyrimidin-5-yl)-2- fluorophenyl)-3-(5-(1- (trifluoromethyl) cyclobutyl)isoxazol- 3-yl)urea |
| Ic-58 | 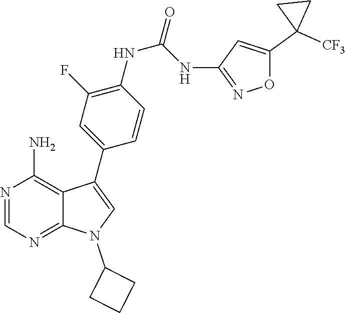 [SMILES: Nc1ncnc2c1c(-c1ccc(NC(=O)Nc3cc(C4(C(F)(F)F)CC4)on3)c(F)c1)cn2C1CCC1] | 1-(4-(4-amino-7- cyclobutyl-7H- pyrrolo[2,3- d]pyrimidin-5-yl)-2- fluorophenyl)-3-(5-(1- (trifluoromethyl) cyclopropyl)isoxazol- 3-yl)urea |
| Ic-59 | 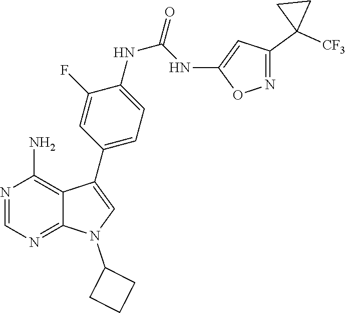 [SMILES: Nc1ncnc2c1c(-c1ccc(NC(=O)Nc3cc(C4(C(F)(F)F)CC4)no3)c(F)c1)cn2C1CCC1] | 1-(6-(4-amino-7- cyclopropyl-7H- pyrrolo[2,3- d]pyrimidin-5- yl)pyridin-3-yl)-3- (3-(1- (trifluoromethyl) cyclopropyl) isoxazol-5-yl)urea |
| Ic-60 | 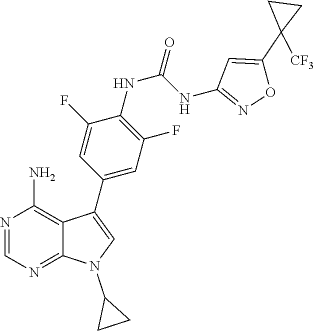 [SMILES: Nc1ncnc2c1c(-c1cc(F)c(NC(=O)Nc3cc(C4(C(F)(F)F)CC4)on3)c(F)c1)cn2C1CC1] | 1-(4-(4-amino-7- cyclopropy-7H- pyrrolo[2,3- d]pyrimidin-5-yl)-2,6- difluorophenyl)-3-(5- (1-(trifluoromethyl) cyclopropyl) isoxazol-3-yl)urea |
| Ic-61 | 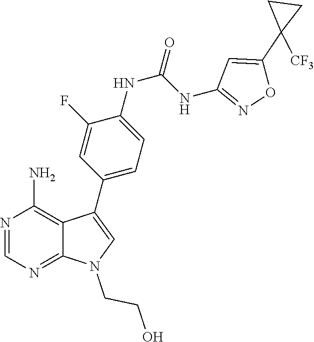 [SMILES: Nc1ncnc2c1c(-c1ccc(NC(=O)Nc3cc(C4(C(F)(F)F)CC4)on3)c(F)c1)cn2CCO] | 1-(4-(4-amino-7-(2- hydroxyethyl)-7H- pyrrolo[2,3- d]pyrimidin-5-yl)-2- fluorophenyl)-3-(5- (1-(trifluoromethyl) cyclopropyl)isoxazol- 3-yl)urea |
| Ic-62 | 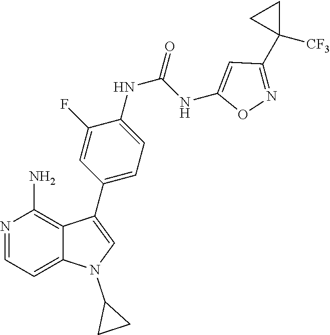 [SMILES: Nc1nccc2c1c(-c1ccc(NC(=O)Nc3cc(C4(C(F)(F)F)CC4)no3)c(F)c1)cn2C1CC1] | 1-(4-(4-amino-1- cyclopropyl-1H- pyrrolo[3,2-c]pyridin- 3-yl)-2-fluorophenyl)- 3-(3-(1- (trifluoromethyl) cyclopropyl)isoxazol- 5-yl)urea |
| Ic-63 | 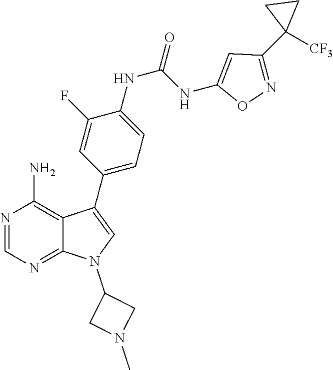 [SMILES: CN1CC(n2cc(-c3ccc(NC(=O)Nc4cc(C5(C(F)(F)F)CC5)no4)c(F)c3)c3c(N)ncnc32)C1] | 1-(4-(4-amino-7-(1- methylazetidin-3-yl)- 7H-pyrrolo[2,3- d]pyrimidin-5-yl)-2- fluorophenyl)-3-(3-(1- (trifluoromethyl) cyclopropyl)isoxazol- 5-yl)urea |
| Ic-64 | 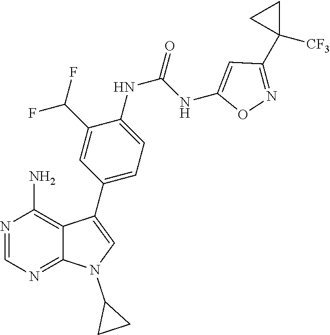 [SMILES: Nc1ncnc2c1c(-c1ccc(NC(=O)Nc3cc(C4(C(F)(F)F)CC4)no3)c(C(F)F)c1)cn2C1CC1] | 1-(4-(4-amino-7- cyclopropyl-7H- pyrrolo[2,3- d]pyrimidin-5-yl)-2- (difluoromethyl) phenyl)-3-(3-(1- (trifluoromethyl) cyclopropyl)isoxazol- 5-yl)urea |
| Ic-65 | 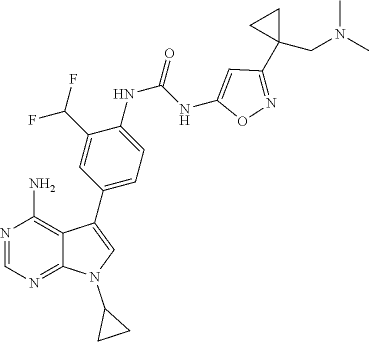 [SMILES: CN(C)CC1(c2cc(NC(=O)Nc3ccc(-c4cn(C5CC5)c5ncnc(N)c45)cc3C(F)F)on2)CC1] | 1-(4-(4-amino-7- cyclopropyl-7H- pyrrolo[2,3- d]pyrimidin-5-yl)-2- fluorophenyl)-3-(3-(1- ((dimethylamino) methyl)cyclopropyl) isoxazol-5-yl)urea |
| Ic-66 | 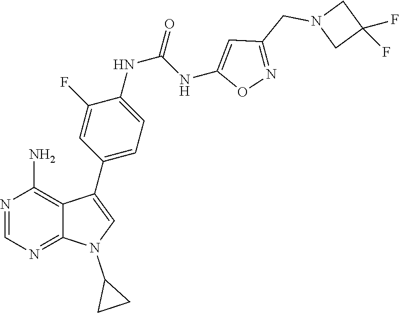 [SMILES: Nc1ncnc2c1c(-c1ccc(NC(=O)Nc3cc(CN4CC(F)(F)C4)no3)c(F)c1)cn2C1CC1] | 1-(4-(4-amino-7- cyclopropyl-7H- pyrrolo[2,3- d]pyrimidin-5-yl)-2- fluorophenyl)-3-(3- ((3,3-difluoroazetidin- 1-yl)methyl)isoxazol-5 yl)urea |

In some embodiments, the NEK7 small molecule inhibitor compound has the following Structure (Id):

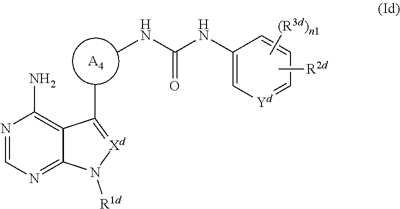

or a pharmaceutically acceptable salt, stereoisomer, or prodrug thereof, wherein:

A4 is C₆-C₁₀ arylene, C₃-C₁₀ cycloalkylene, 3-10 membered heterocyclylene, or 5-6 membered heteroarylene;

X^{d} is N or CR^{4d};

Y^{d} is N or CH;

R^{1d} is C₁-C₆ alkyl, C₁-C₆ hydroxylalkyl, C₃-C₁₀ cycloalkyl, or 3-10 membered heterocyclyl;

R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;

R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl;

R^{4d} is H, C₁-C₆ alkyl, C₁-C₆ haloalkyl, or C₃-C₈ cycloalkyl; and

n1 is 0, 1, 2, 3, or 4.

In some embodiments, A4 is C₆-C₁₀ arylene. In some specific embodiments, A4 is phenylene. In certain embodiments, A4 is 5-6 membered heteroarylene. In certain specific embodiments, A4 is pyridiylene. In some embodiments, A4 is C₃-C₁₀ cycloalkylene or 3-10 membered heterocyclylene. In more specific embodiments, A4 is substituted with one or more substituents selected from halo, cyano, C₁-C₆ alkyl, C₁-C₆ haloalkyl, C₁-C₆ alkoxy, C₁-C₆ haloalkoxy, or C₃-C₈ cycloalkyl. In certain more specific embodiments, A4 is substituted with one or more halo substituents. In other embodiments, A4 is unsubstituted.

In certain embodiments, X^{d} is CR^{4d}. In more specific embodiments, R^{4d} is H or C₁-C₆ alkyl. In some embodiments, R^{4d} is H. In certain specific embodiments, X^{d} is N.

In some embodiments, R^{1d} is C₁-C₆ alkyl. In some more specific embodiments, R^{1d} is methyl or iso-propyl. In certain embodiments, R^{1d} is C₁-C₆ hydroxylalkyl. In certain more specific embodiments, R^{1d} has one of the following structures:

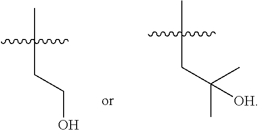

In some embodiments, R^{1d} is C₃-C₁₀ cycloalkyl. In more specific embodiments, R^{1d} is cyclopropyl or cyclobutyl. In certain embodiments, R^{1d} is 3-10 membered heterocyclyl. In certain embodiments, R^{1d} is oxetanyl, pyrrolidinyl, or piperidinyl.

In some embodiments, R^{1d} is substituted with one or more substituents selected from halo, cyano, C₁-C₆ alkyl, C₁-C₆ haloalkyl, C₁-C₆ alkoxy, C₁-C₆ haloalkoxy, or C₃-C₈ cycloalkyl. In more specific embodiments, R^{1d} is substituted with one or more C₁-C₆ alkyl substituents. In other embodiments, R^{1d} is unsubstituted.

In certain specific embodiments, R^{1d} has one of the following structures:

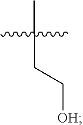 [SMILES: *CCO]

### —CH₃;

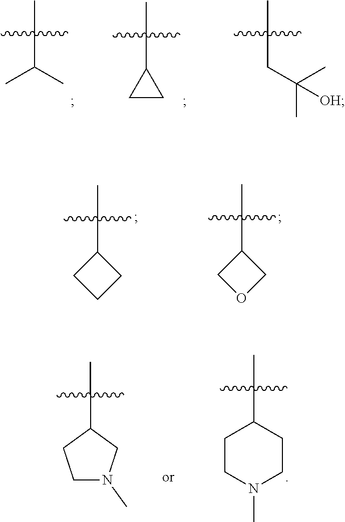

In some specific embodiments, the compound has the following Structure (Id-Ia):

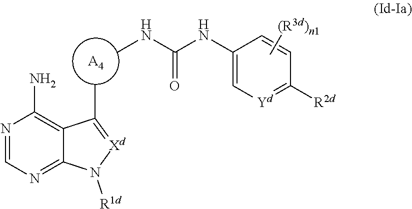

or a pharmaceutically acceptable salt, stereoisomer, or prodrug thereof.

In some embodiments, Y^{d} is N. In other embodiments, Y^{d} is CH.

In certain specific embodiments, the compound has the following Structure (Id-Ib):

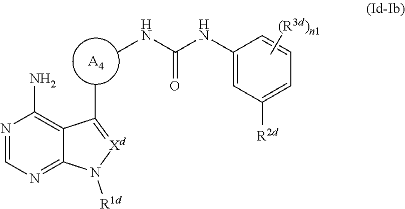

or a pharmaceutically acceptable salt, stereoisomer, or prodrug thereof.

In some embodiments, R^{2d} is a 3-10 membered heterocyclyl. In some specific embodiments, R^{2d} is morpholino. In other specific embodiments, R^{2d} is piperazinyl. In some more specific embodiments, R^{2d} has one of the following structures:

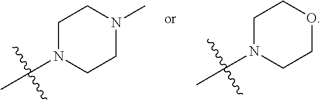

In some embodiments, R^{2d} is a 3-10 membered heterocyclylalkyl. In certain more specific embodiments, R^{2d} has one of the following structures:

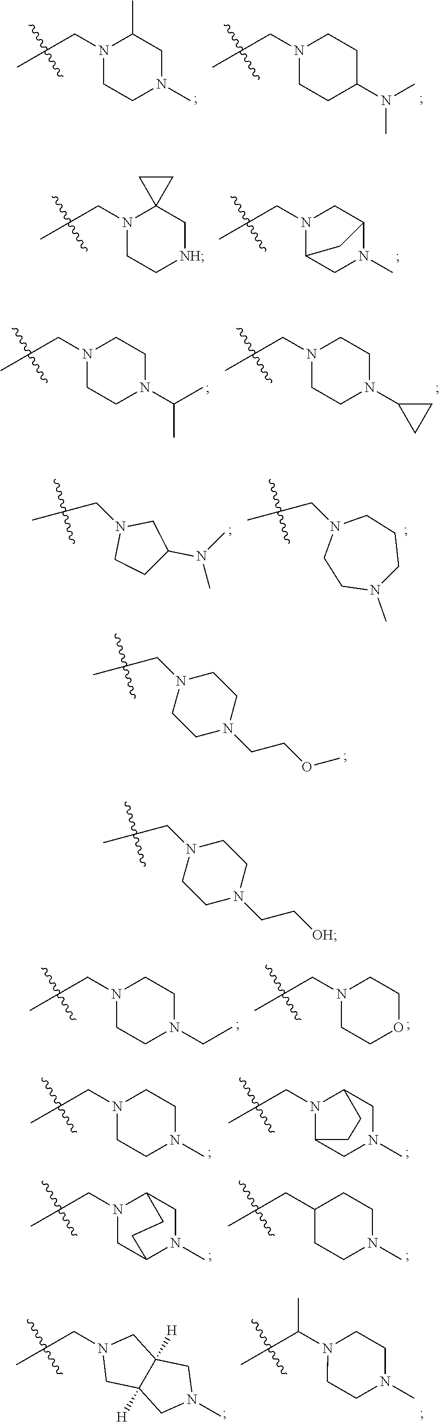

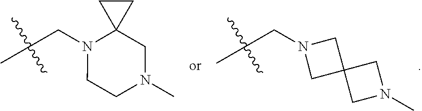

In some embodiments, R^{2d} is 3-10 membered heterocyclylcarbonyl. In certain more specific embodiments, R^{2d} has the following structure:

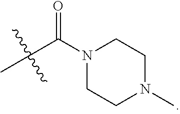 [SMILES: *C(=O)N1CCN(C)CC1]

In some embodiments, R^{2d} is a 5-6 membered heteroaryl. In some more specific embodiments, R^{2d} has the following structure:

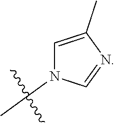 [SMILES: *n1cnc(C)c1]

In some embodiments, R^{2d} is 3-10 membered heterocyclyloxy. In certain more specific embodiments, R^{2d} has the following structure:

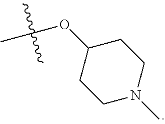 [SMILES: *OC1CCN(C)CC1]

In some more specific embodiments, R^{2d} has the following structure:

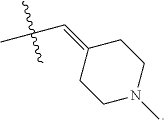 [SMILES: *C=C1CCN(C)CC1]

In some embodiments, n1 is 0. In some embodiments, n1 is 1 or 2. In certain embodiments, n1 is 1. In some embodiments, R^{3d} is halo, cyano, C₁-C₆ alkyl, C₁-C₆ haloalkyl, C₁-C₆ alkoxy, C₁-C₆ haloalkoxy, or C₃-C₈ cycloalkyl. In certain specific embodiments, R^{3d} is methyl, chloro, fluoro, cyano, difluoromethyl, trifluoromethyl, methoxy, trifluoromethoxy, or cyclopropyl. In some specific embodiments, n1 is 1 or 2 and R^{3d} is halo, C₁-C₆ alkyl, C₂-C₆ haloalkyl, C₁-C₆ alkoxy, C₁-C₆ haloalkoxy, or C₃-C₈ cycloalkyl. In some embodiments, n1 is 1 and R^{3d} is halo, C₁-C₆ alkyl, C₁-C₆ haloalkyl, C₁-C₆ alkoxy, C₁-C₆ haloalkoxy, or C₃-C₈ cycloalkyl. In some embodiments, n1 is 1 and R^{3d} is methyl, chloro, fluoro, trifluoromethyl, methoxy, trifluoromethoxy, or cyclopropyl.

In various different embodiments, the compound has one of the structures set forth in Table 1d below, or a pharmaceutically acceptable salt, stereoisomer, or prodrug thereof. Compounds in Table 1d were prepared as described in the Examples or methods known in the art and analyzed by mass spectrometry and/or ¹H NMR.

TABLE 1d
Representative compounds of Structure (Id)

| Cmp | Structure | Name |
|---|---|---|
| Id-1 | 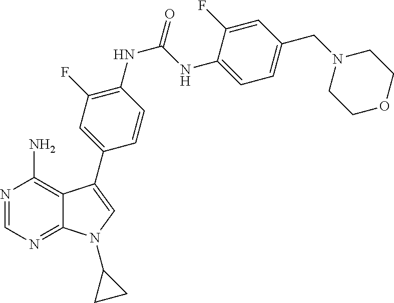 [SMILES: Nc1ncnc2c1c(-c1ccc(NC(=O)Nc3ccc(CN4CCOCC4)cc3F)c(F)c1)cn2C1CC1] | 1-(4-(4-amino-7- cyclopropyl-7H- pyrrolo[2,3- d]pyrimidin-5-yl)-2- fluorophenyl)-3-(2- fluoro-4- (morpholinomethyl) phenyl)urea |
| Id-2 | 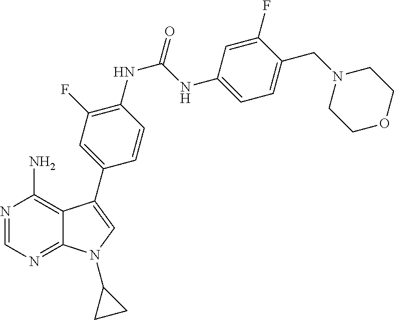 [SMILES: Nc1ncnc2c1c(-c1ccc(NC(=O)Nc3ccc(CN4CCOCC4)c(F)c3)c(F)c1)cn2C1CC1] | 1-(4-(4-amino-7- cyclopropyl-7H- pyrrolo[2,3- dlpyrimidin-5-yl)-2- fluorophenyl)-3-(3- fluoro-4- (morpholinomethyl) phenyl)urea |
| Id-3 | 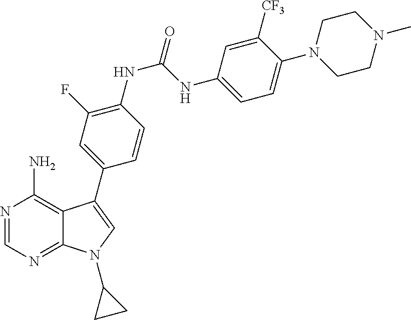 [SMILES: CN1CCN(c2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-(4- methylpiperazin-1-yl)- 3-(trifluoromethyl) phenyl)urea |
| Id-4 | 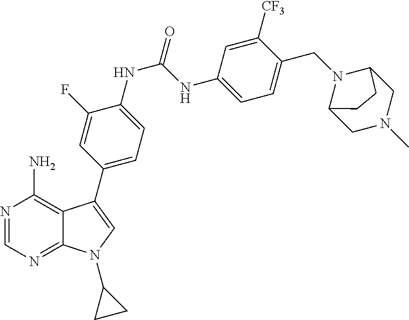 [SMILES: CN1CC2CCC(C1)N2Cc1ccc(NC(=O)Nc2ccc(-c3cn(C4CC4)c4ncnc(N)c34)cc2F)cc1C(F)(F)F] | 1-(4-(4-amino-7- cyclopropyl-7H- pyrrolo[2,3- d]pyrimidin-5-yl)-2- floorophenyl))-3-(4-((3- methyl-3,8- diazablcyclo[3.2.1] octan-8-yl)methyl)-3- (trifluoromethyl) phenyl)urea |
| Id-5 | 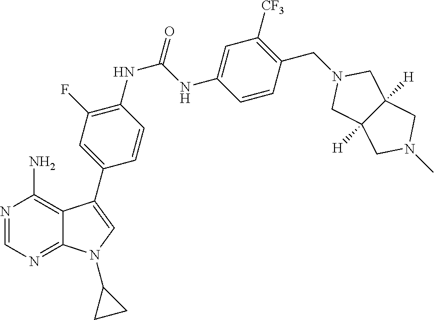 [SMILES: CN1C[C@@H]2CN(Cc3ccc(NC(=O)Nc4ccc(-c5cn(C6CC6)c6ncnc(N)c56)cc4F)cc3C(F)(F)F)C[C@@H]2C1] | 1-(4-(4-amino-7- cyclopropyl-7H- pyrrolo[2,3- d]pyrimidin-5-yl)-2- fluorophenyl)-3-(4- (((3aR,6aS)-5- methylhexahydropyrrolo [3,4-c]pyrrol-2(1H)- yl)methyl)-3- (trifluoromethyl) phenyl)urea |
| Id-6 | 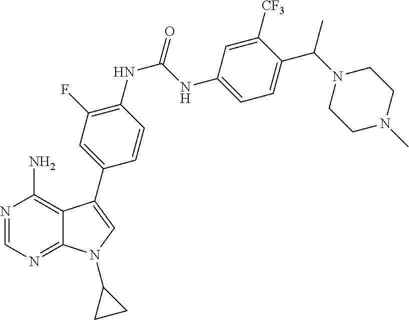 [SMILES: CC(c1ccc(NC(=O)Nc2ccc(-c3cn(C4CC4)c4ncnc(N)c34)cc2F)cc1C(F)(F)F)N1CCN(C)CC1] | 1-(4-(4-amino-7- cyclopropyl-7H- pyrrolo[2,3- d]pyrimidin-5-yl)-2- fluorophenyl)-3-(4- (1-(4-methylpiperazin-1- yl)ethyl)-3- (trifluoromethyl) phenyl)urea |
| Id-7 | 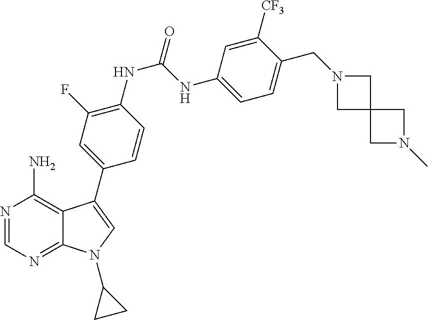 [SMILES: CN1CC2(C1)CN(Cc1ccc(NC(=O)Nc3ccc(-c4cn(C5CC5)c5ncnc(N)c45)cc3F)cc1C(F)(F)F)C2] | 1-(4-(4-amino-7- cyclopropyl-7H- pyrrolo[2,3- d]pyrimidin-5-yl)-2- fluorophenyl)-3-(4- ((6-methyl-2,6- diazaspiro[3.3]heptan- 2-yl)methyl)-3- (trifluoromethyl) phenyl)urea |
| Id-8 | 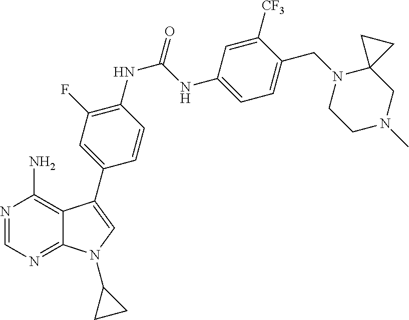 [SMILES: CN1CCN(Cc2ccc(NC(=O)Nc3ccc(-c4cn(C5CC5)c5ncnc(N)c45)cc3F)cc2C(F)(F)F)C2(CC2)C1] | 1-(4-(4-amino-7- cyclopropyl-7H- pyrrolo[2,3- d]pyrimidin-5-yl)-2- fluorophenyl)-3-(4-((7- methyl-4,7- diazaspiro[2.5]octan-4- yl)methyl)-3- (trifluoromethyl) phenyl)urea |
| Id-9 | 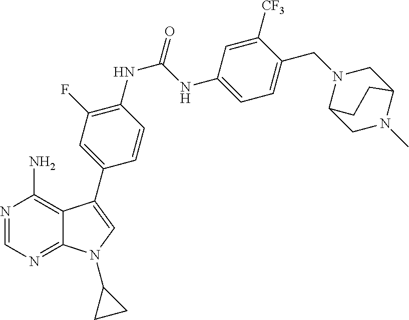 [SMILES: CN1CC2CCC1CN2Cc1ccc(NC(=O)Nc2ccc(-c3cn(C4CC4)c4ncnc(N)c34)cc2F)cc1C(F)(F)F] | 1-(4-(4-amino-7- cyclopropyl-7H- pyrrolo[2,3- d]pyrimidin-5-yl)-2- fluorophenyl)-3-(4-((5- methyl-2,5- diazaspiro[2.2.2]octan- 2-yl)methyl)-3- (trifluoromethyl) phenyl)urea |
| Id-10 | 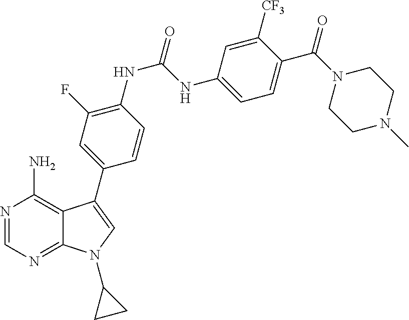 [SMILES: CN1CCN(C(=O)c2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-(4- methylpiperazine-1- carbonyl)-3- (trifluoromethyl) phenyl)urea |
| Id-11 | 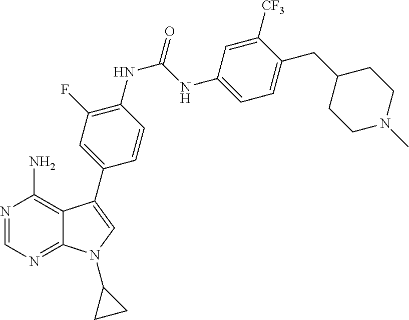 [SMILES: CN1CCC(Cc2ccc(NC(=O)Nc3ccc(-c4cn(C5CC5)c5ncnc(N)c45)cc3F)cc2C(F)(F)F)CC1] | 1-(4-(4-amino-7- cyclopropy]-7H- pyrrolo[2,3- d]pyrimidin-5-yl)-2- fluorophenyl)-3-(4-((1- methylpiperidin-4- yl)methyl)-3- (trifluoromethyl) phenyl)urea |
| Id-12 | 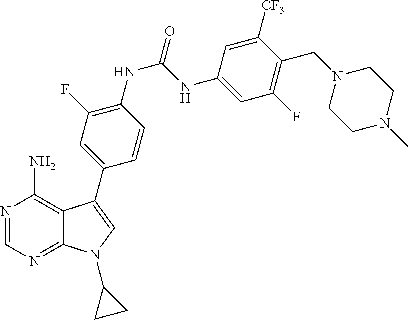 [SMILES: CN1CCN(Cc2c(F)cc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(3- fluoro-4-((4- methylpiperazin-1- yl)methyl)-5- (trifluoromethyl) phenyl)urea |
| Id-13 | 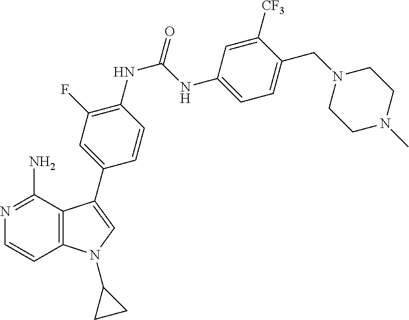 [SMILES: CN1CCN(Cc2ccc(NC(=O)Nc3ccc(-c4cn(C5CC5)c5ccnc(N)c45)cc3F)cc2C(F)(F)F)CC1] | 1-(4-(4-amino-1- cyclopropyl-1H- pyrrolo[3,2-c]pyridin-3- yl)-2-fluorophenyl)-3- (4-((4-methylpiperazin- 1-yl)methyl)-3- (trifluoromethyl) phenyl)urea |
| Id-14 | 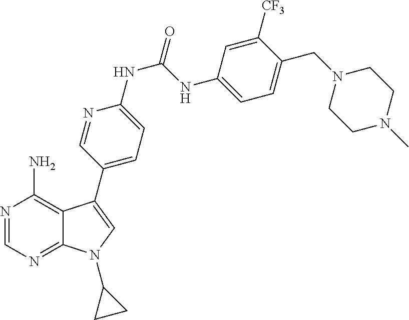 [SMILES: CN1CCN(Cc2ccc(NC(=O)Nc3ccc(-c4cn(C5CC5)c5ncnc(N)c45)cn3)cc2C(F)(F)F)CC1] | 1-(5-(4-amino-7- cyclopropyl-7H- pyrrolo[2,3- d]pyrimidin-5- yl)pyridin-2-yl)-3-(4- ((4-methylpiperazin-1- yl)methyl)-3- (trifluoromethyl) phenyl)urea |
| Id-15 | 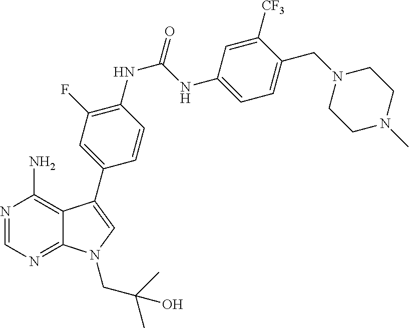 [SMILES: CN1CCN(Cc2ccc(NC(=O)Nc3ccc(-c4cn(CC(C)(C)O)c5ncnc(N)c45)cc3F)cc2C(F)(F)F)CC1] | 1-(4-(4-amino-7-(2- hydroxy-2- methylpropyl)-7H- pyrrolo[2,3- d]pyrimidin-5-yl)-2- fluorophenyl)-3-(4-((4- methylpiperazin-1- yl)methyl)-3- (trifluoromethyl) phenyl)urea |
| Id-16 | 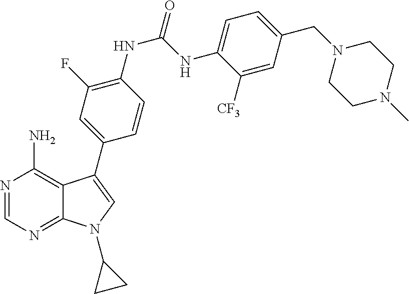 [SMILES: CN1CCN(Cc2ccc(NC(=O)Nc3ccc(-c4cn(C5CC5)c5ncnc(N)c45)cc3F)c(C(F)(F)F)c2)CC1] | 1-(4-(4-amino-7- cyclopropyl-7H- pyrrolo[2,3- d]pyrimidin-5-yl)-2- fluorophenyl)-3-(4-((4- methylpiperazin-1- yl)methyl)-2- (trifluoromethyl) phenyl)urea |
| Id-17 | 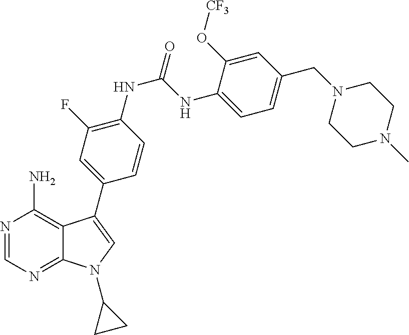 [SMILES: CN1CCN(Cc2ccc(NC(=O)Nc3ccc(-c4cn(C5CC5)c5ncnc(N)c45)cc3F)c(OC(F)(F)F)c2)CC1] | 1-(4-(4-amino-7- cyclopropyl-7H- pyrrolo[2,3- d]pyrimidin-5-yl)-2- fluorophenyl)-3-(4-((4- methylpiperazin-1- yl)methyl)-2- (trifluoromethyl) phenyl)urea |
| Id-18 | 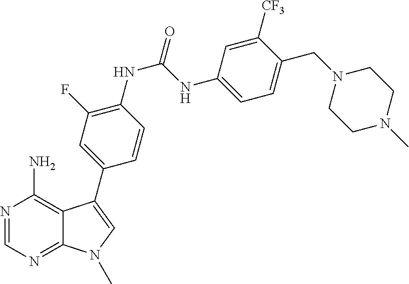 [SMILES: CN1CCN(Cc2ccc(NC(=O)Nc3ccc(-c4cn(C)c5ncnc(N)c45)cc3F)cc2C(F)(F)F)CC1] | 1-(4-(4-amino-7- methyl-7H-pyrrolo[2,3- d]pyrimidin-5-yl)-2- fluorophenyl)-3-(4-((4- methylpiperazin-1- yl)methyl)-3- (trifluoromethyl) phenyl)urea |
| Id-19 | 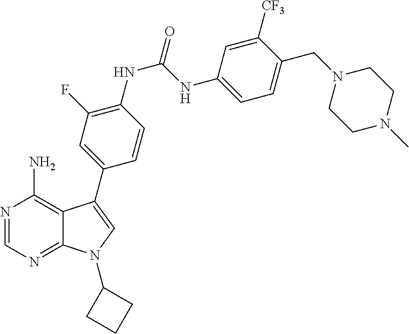 [SMILES: CN1CCN(Cc2ccc(NC(=O)Nc3ccc(-c4cn(C5CCC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((4- methylpiperazin-1- yl)methyl)-3- (trifluoromethyl) phenyl)urea |
| Id-20 | 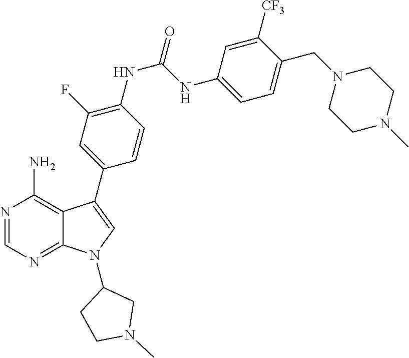 [SMILES: CN1CCN(Cc2ccc(NC(=O)Nc3ccc(-c4cn(C5CCN(C)C5)c5ncnc(N)c45)cc3F)cc2C(F)(F)F)CC1] | 1-(4-(4-amino-7-(1- methylpyrrolidin-3-yl)- 7H-pyrrolo[2,3- d]pyrimidin-5-yl)-2- fluorophenyl)-3-(4-((4- methylpiperazin-1- yl)methyl)-2- (trifluoromethyl) phenyl)urea |
| Id-21 | 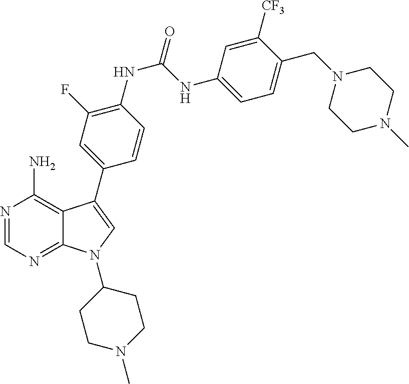 [SMILES: CN1CCC(n2cc(-c3ccc(NC(=O)Nc4ccc(CN5CCN(C)CC5)c(C(F)(F)F)c4)c(F)c3)c3c(N)ncnc32)CC1] | 1-(4-(4-amino-7-(1- methylpiperidin-4-yl)- 7H-pyrrolo[2,3- d]pyrimidin-5-yl)-2- fluorophenyl))-3-(4-((4- methylpiperazin-1- yl)methyl)-3- (trifluoromethyl) phenyl)urea |
| Id-22 | 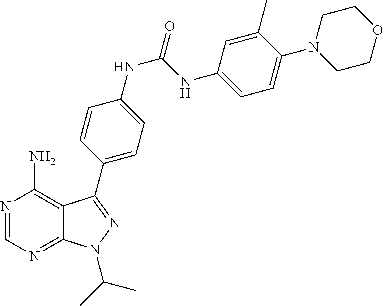 [SMILES: Cc1cc(NC(=O)Nc2ccc(-c3nn(C(C)C)c4ncnc(N)c34)cc2)ccc1N1CCOCC1] | 1-(4-(4-amino-1- isopropyl-1H- pyrazolo[3,4- d]pyrimidin-3- yl)phenyl)-3-(3-methyl-4- morpholinophenyl)urea |
| Id-23 | 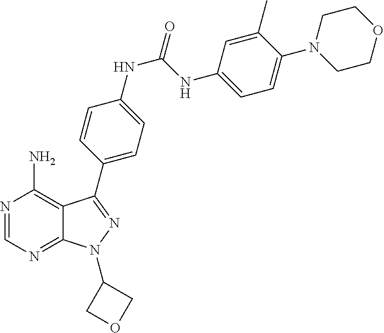 [SMILES: Cc1cc(NC(=O)Nc2ccc(-c3nn(C4COC4)c4ncnc(N)c34)cc2)ccc1N1CCOCC1] | 1-(4-(4-amino-1- (oxetan-3-yl)-1H- pyrazolo[3,4- d]pyrimidin-3- yl)phenyl)-3-(3-methyl-4- morpholinophenyl)urea |
| Id-24 | 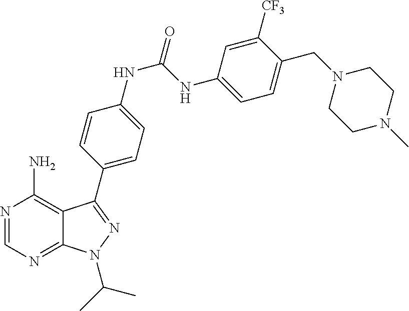 [SMILES: CC(C)n1nc(-c2ccc(NC(=O)Nc3ccc(CN4CCN(C)CC4)c(C(F)(F)F)c3)cc2)c2c(N)ncnc21] | 1-(4-(4-amino-1- isopropyl-1H- pyrazolo[3,4- d]pyrimidin-3- yl)phenyl)-3-(4-((4- methylpiperazin-1- yl)methyl)-3- (trifluoromethyl) phenyl)urea |
| Id-25 | 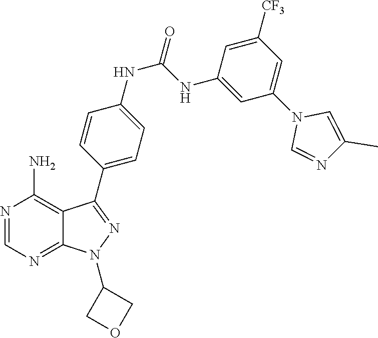 [SMILES: Cc1cn(-c2cc(NC(=O)Nc3ccc(-c4nn(C5COC5)c5ncnc(N)c45)cc3)cc(C(F)(F)F)c2)cn1] | 1-(4-(4-amino-1- (oxetan-3-yl)-1H- pyrazolo[3,4- d]pyrimidin-3- yl)phenyl)-3-(3-(4- methyl-1H-imidazol-1- yl)-5- (trifluoromethyl) phenyl)urea |
| Id-26 | 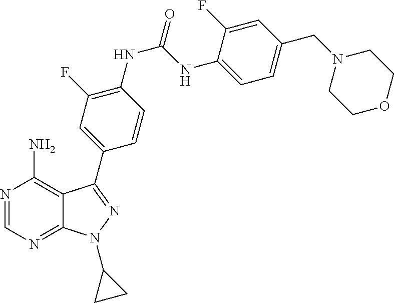 [SMILES: Nc1ncnc2c1c(-c1ccc(NC(=O)Nc3ccc(CN4CCOCC4)cc3F)c(F)c1)nn2C1CC1] | 1-(4-(4-amino-1- cyclopropyl-1H- pyrazolo[3,4- d]pyrimidin-3-yl)-2- fluorophenyl)-3-(2- fluoro-4- (morpholinomethyl) phenyl)urea |
| Id-27 | 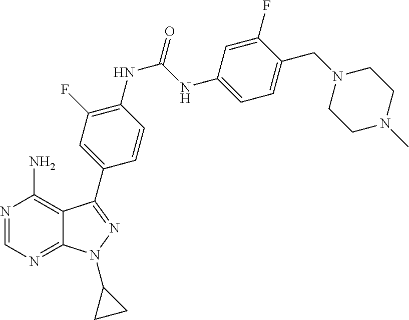 [SMILES: CN1CCN(Cc2ccc(NC(=O)Nc3ccc(-c4nn(C5CC5)c5ncnc(N)c45)cc3F)cc2F)CC1] | 1-(4-(4-amino-1- cyclopropyl-1H- pyrazolo[3,4- d]pyrimidin-3-yl)-2- fluorophenyl)-3-(3- fluoro-4-((4- methylpiperazin-1- yl)methyl)phenyl)urea |
| Id-28 | 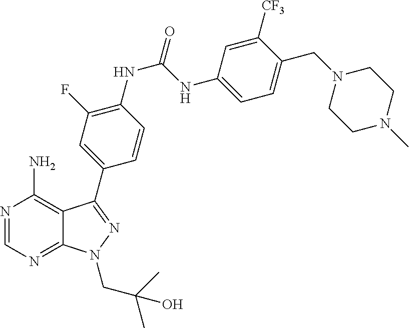 [SMILES: CN1CCN(Cc2ccc(NC(=O)Nc3ccc(-c4nn(CC(C)(C)O)c5ncnc(N)c45)cc3F)cc2C(F)(F)F)CC1] | 1-(4-(4-amino-1-(2- hydroxy-2- methylpropyl)-1H- pyrazolo[3,4- d]pyrimidin-3-yl)-2- fluorophenyl)-3-(4-((4- methylpiperazin-1- yl)methyl)-3- (trifluoromethyl) phenyl)urea |
| Id-29 | 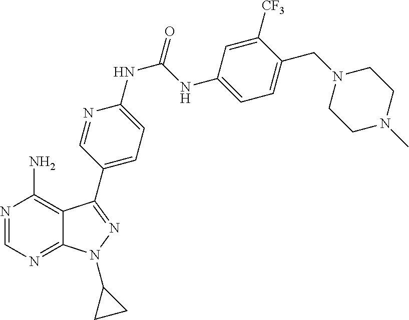 [SMILES: CN1CCN(Cc2ccc(NC(=O)Nc3ccc(-c4nn(C5CC5)c5ncnc(N)c45)cn3)cc2C(F)(F)F)CC1] | 1-(5-(4-amino-1- cyclopropyl-1H- pyrazolo[3,4- d]pyrimidin-3- yl)pyridin-2-yl)-3-(4- ((4-methylpiperazin-1- yl)methyl)-3- (trifluoromethyl) phenyl)urea |
| Id-30 | 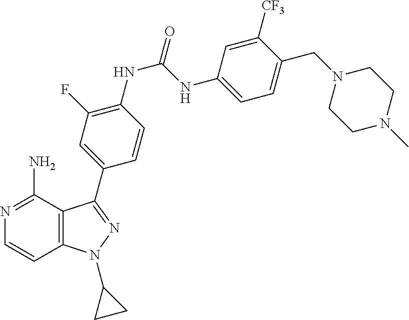 [SMILES: CN1CCN(Cc2ccc(NC(=O)Nc3ccc(-c4nn(C5CC5)c5ccnc(N)c45)cc3F)cc2C(F)(F)F)CC1] | 1-(4-(4-amino-1- cyclopropyl-1H- pyrazolo[4,3-c]pyridin- 3-yl)-2-fluorophenyl)- 3-(4-((4- methylpiperazin-1- yl)methyl)-3- (trifluoromethyl) phenyl)urea |
| Id-31 | 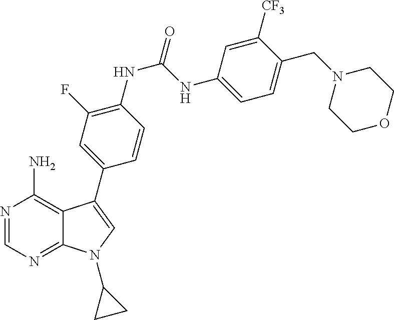 [SMILES: Nc1ncnc2c1c(-c1ccc(NC(=O)Nc3ccc(CN4CCOCC4)c(C(F)(F)F)c3)c(F)c1)cn2C1CC1] | 1-(4-(4-amino-7- cyclopropyl-7H- pyrrolo[2,3- d]pyrimidin-5-yl)-2- fluorophenyl)-3-(4- (morpholinomethyl)-3- (trifluoromethyl) phenyl)urea |
| Id-32 | 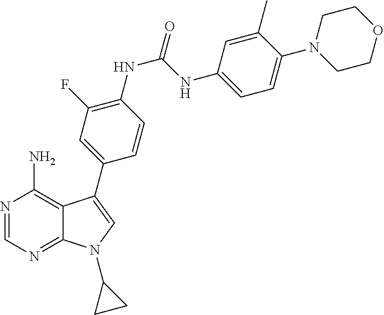 [SMILES: Cc1cc(NC(=O)Nc2ccc(-c3cn(C4CC4)c4ncnc(N)c34)cc2F)ccc1N1CCOCC1] | 1-(4-(4-amino-7- cyclopropyl-7H- pyrrolo[2,3- d]pyrimidin-5-yl)-2- fluorophenyl)-3-(3- methyl-4- morpholinophenyl)urea |
| Id-33 | 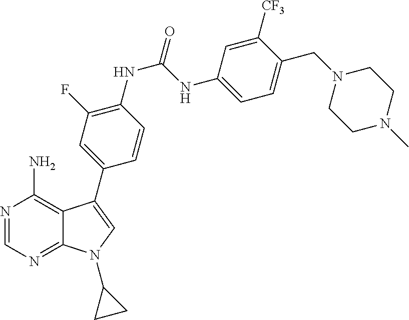 [SMILES: CN1CCN(Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((4- methylpiperazin-1- yl)methyl)-3- (trifluoromethyl) phenyl)urea |
| Id-34 | 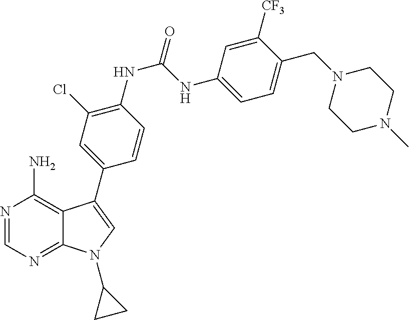 [SMILES: CN1CCN(Cc2ccc(NC(=O)Nc3ccc(-c4cn(C5CC5)c5ncnc(N)c45)cc3Cl)cc2C(F)(F)F)CC1] | 1-(4-(4-amino-7- cyclopropyl-7H- pyrrolo[2,3- d]pyrimidin-5-yl)-2- chlorophenyl)-3-(4-((4- methylpiperazin-1- yl)methyl)-3- (trifluoromethyl) phenyl)urea |
| Id-35 | 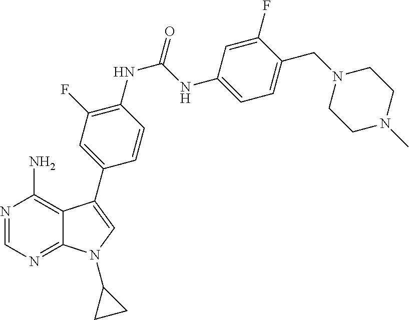 [SMILES: CN1CCN(Cc2ccc(NC(=O)Nc3ccc(-c4cn(C5CC5)c5ncnc(N)c45)cc3F)cc2F)CC1] | 1-(4-(4-amino-7- cyclopropyl-7H- pyrrolo[2,3- d]pyrimidin-5-yl)-2- fluorophenyl)-3-(3- fluoro-4-((4- methylpiperazin-1- yl)methyl)phenyl)urea |
| Id-36 | 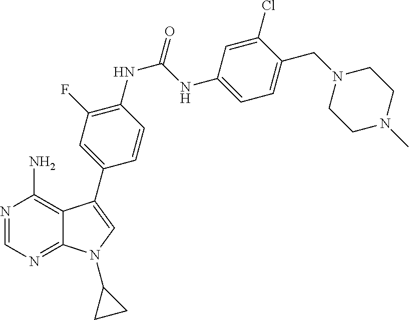 [SMILES: CN1CCN(Cc2ccc(NC(=O)Nc3ccc(-c4cn(C5CC5)c5ncnc(N)c45)cc3F)cc2Cl)CC1] | 1-(4-(4-amino-7- cyclopropyl-7H- pyrrolo[2,3- d]pyrimidin-5-yl)-2- fluorophenyl)-3-(3- chloro-4-((4- methylpiperazin-1- yl)methyl)phenyl)urea |
| Id-37 | 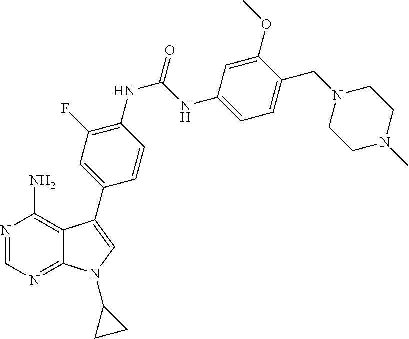 [SMILES: COc1cc(NC(=O)Nc2ccc(-c3cn(C4CC4)c4ncnc(N)c34)cc2F)ccc1CN1CCN(C)CC1] | 1-(4-(4-amino-7- cyclopropyl-7H- pyrrolo[2,3- d]pyrimidin-5-yl)-2- fluorophenyl)-3-(3- methoxy-4-((4- methylpiperazin-1- yl)methyl)phenyl)urea |
| Id-38 | 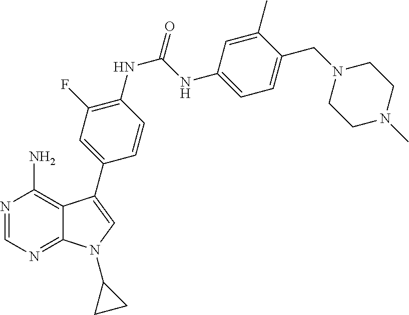 [SMILES: Cc1cc(NC(=O)Nc2ccc(-c3cn(C4CC4)c4ncnc(N)c34)cc2F)ccc1CN1CCN(C)CC1] | 1-(4-(4-amino-7- cyclopropyl-7H- pyrrolo[2,3- d]pyrimidin-5-yl)-2- fluorophenyl)-3-(3- methoxy-4-((4- methylpiperazin-1- yl)methyl)phenyl)urea |
| Id-39 | 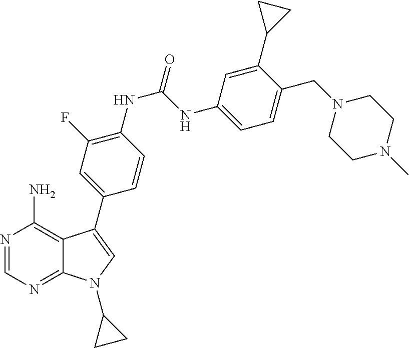 [SMILES: CN1CCN(Cc2ccc(NC(=O)Nc3ccc(-c4cn(C5CC5)c5ncnc(N)c45)cc3F)cc2C2CC2)CC1] | 1-(4-(4-amino-7- cyclopropyl-7H- pyrrolo[2,3- d]pyrimidin-5-yl)-2- fluorophenyl)-3-(3- cyclopropyl-4-((4- methylpiperazin-1- yl)methyl)phenyl)urea |
| Id-40 | 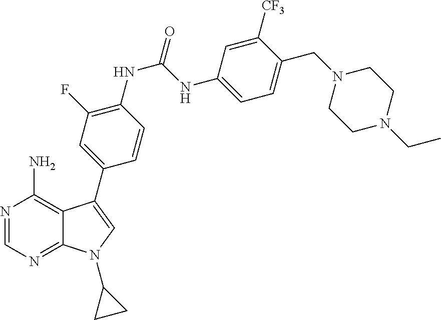 [SMILES: CCN1CCN(Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((4- ethylpiperazin-1- yl)methyl)-3- (trifluoromethyl) phenyl)urea |
| Id-41 | 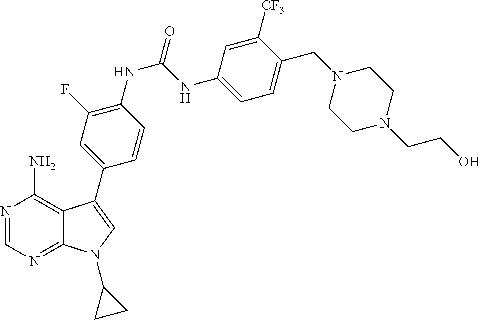 [SMILES: Nc1ncnc2c1c(-c1ccc(NC(=O)Nc3ccc(CN4CCN(CCO)CC4)c(C(F)(F)F)c3)c(F)c1)cn2C1CC1] | 1-(4-(4-amino-7- cyclopropyl-7H- pyrrolo[2,3- d]pyrimidin-5-yl)-2- fluorophenyl)-3-(4-((4- (2-hydroxyethyl)piperazin- 1-yl)methyl)-3- (trifluoromethyl) phenyl)urea |
| Id-42 | 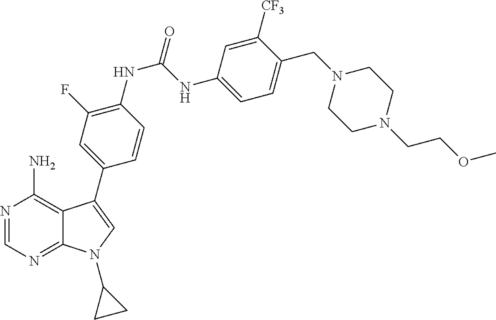 [SMILES: COCCN1CCN(Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((4- (2-methoxyethyl)piperazin- 1-yl)methyl)-3- (trifluoromethyl) phenyl)urea |
| Id-43 | 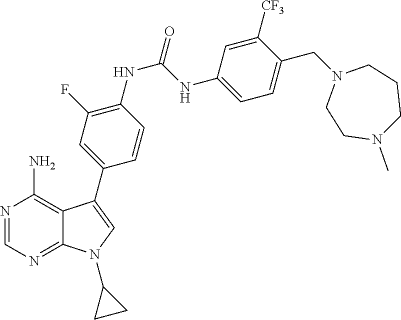 [SMILES: CN1CCCN(Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((4- methyl-1,4-diazepan-1- yl)methyl)-3- (trifluoromethyl) phenyl)urea |
| Id-44 | 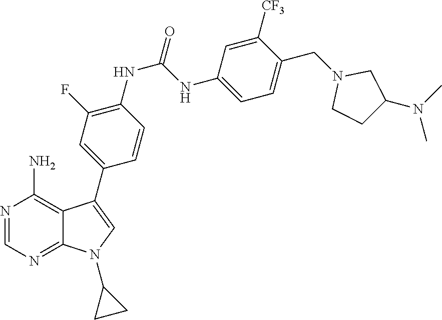 [SMILES: CN(C)C1CCN(Cc2ccc(NC(=O)Nc3ccc(-c4cn(C5CC5)c5ncnc(N)c45)cc3F)cc2C(F)(F)F)C1] | 1-(4-(4-amino-7- cyclopropyl-7H- pyrrolo[2,3- d]pyrimidin-5-yl)-2- fluorophenyl)-3-(4-((3- (dimethylamino)pyrrolidin- 1-yl)methyl)-3- (trifluoromethyl) phenyl)urea |
| Id-45 | 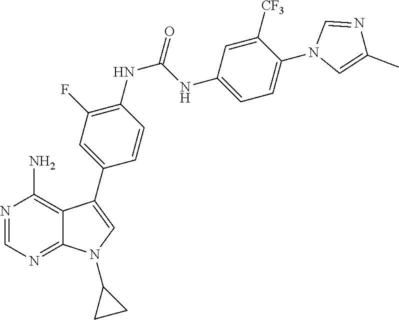 [SMILES: Cc1cn(-c2ccc(NC(=O)Nc3ccc(-c4cn(C5CC5)c5ncnc(N)c45)cc3F)cc2C(F)(F)F)cn1] | 1-(4-(4-amino-7- cyclopropyl-7H- pyrrolo[2,3- d]pyrimidin-5-yl)-2- fluorophenyl)-3-(4-(4- methyl-1H-imidazol-1- yl)-3- (trifluoromethyl) phenyl)urea |
| Id-46 | 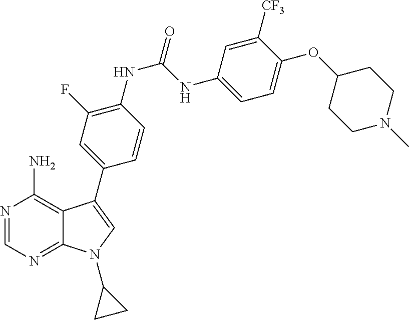 [SMILES: CN1CCC(Oc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((1- methylpiperidin-4- yl)oxy)-3- (trifluoromethyl) phenyl)urea |
| Id-47 | 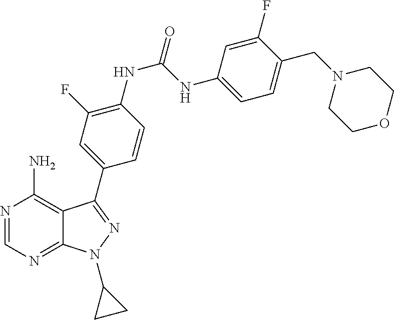 [SMILES: Nc1ncnc2c1c(-c1ccc(NC(=O)Nc3ccc(CN4CCOCC4)c(F)c3)c(F)c1)nn2C1CC1] | 1-(4-(4-amino-1- cyclopropyl-1H- pyrazolo[3,4- d]pyrimidin-3-yl)-2- fluorophenyl)-3-(3- fluoro-4- (morpholinomethyl) phenyl)urea |
| Id-48 | 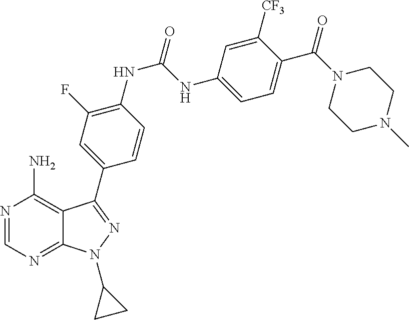 [SMILES: CN1CCN(C(=O)c2ccc(NC(=O)Nc3ccc(-c4nn(C5CC5)c5ncnc(N)c45)cc3F)cc2C(F)(F)F)CC1] | 1-(4-(4-amino-1- cyclopropyl-1H- pyrazolo[3,4- d]pyrimidin)-3-yl)-2- fluorophenyl)-3-(4-(4- methylpiperazine-1- carbonyl)-3- (trifluoromethyl) phenyl)urea |
| Id-49 | 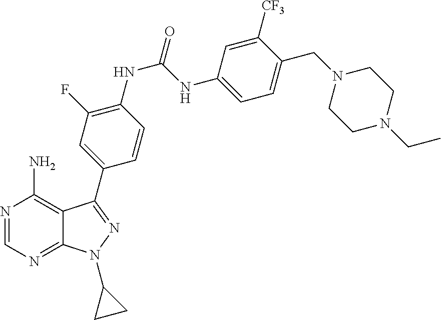 [SMILES: CCN1CCN(Cc2ccc(NC(=O)Nc3ccc(-c4nn(C5CC5)c5ncnc(N)c45)cc3F)cc2C(F)(F)F)CC1] | 1-(4-(4-amino-1- cyclopropyl-1H- pyrazolo[3,4- d]pyrimidin)-3-yl)-2- fluorophenyl)-3-(4-((4- ethylpiperazin-1- yl)methyl)-3- (trifluoromethyl) phenyl)urea |
| Id-50 | 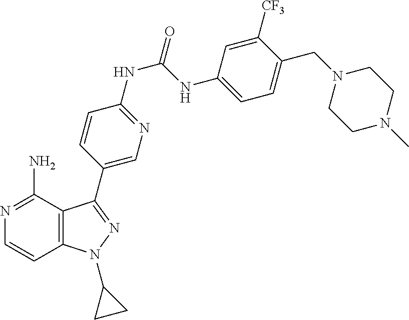 [SMILES: CN1CCN(Cc2ccc(NC(=O)Nc3ccc(-c4nn(C5CC5)c5ccnc(N)c45)cn3)cc2C(F)(F)F)CC1] | 1-(5-(4-amino-1- cyclopropyl-1H- pyrazolo[4,3-c]pyridin- 3-yl)pyridin-2-yl)-3-(4- ((4-methylpiperazin-1- yl)methyl)-3- (trifluoromethyl) phenyl)urea |
| Id-51 | 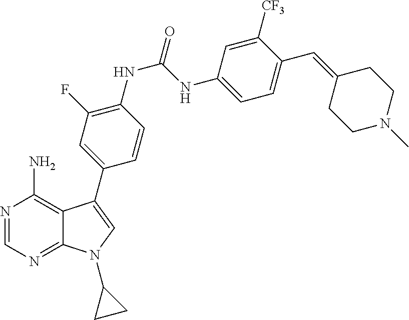 [SMILES: CN1CCC(=Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin-5-yl)-2- fluorophenyl)-3-(4-((1- methylpiperidin-4- ylidene)methyl)-3- (trifluoromethyl) phenyl)urea |
| Id-52 | 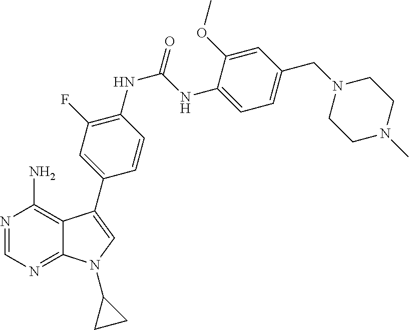 [SMILES: COc1cc(CN2CCN(C)CC2)ccc1NC(=O)Nc1ccc(-c2cn(C3CC3)c3ncnc(N)c23)cc1F] | 1-(4-(4-amino-7- cyclopropyl-7H- pyrrolo[2,3- d]pyrimidin-5-yl)-2- fluorophenyl)-3-(2- methoxy-4-((4- methylpiperazin-1- yl)methyl)phenyl)urea |
| Id-53 | 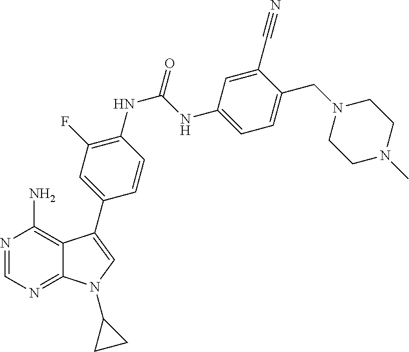 [SMILES: CN1CCN(Cc2ccc(NC(=O)Nc3ccc(-c4cn(C5CC5)c5ncnc(N)c45)cc3F)cc2C#N)CC1] | 1-(4-(4-amino-7- cyclopropyl-7H- pyrrolo[2,3- d]pyrimidin-5-yl)-2- fluorophenyl)-3-(3- cyano-4-((4- methylpiperazin-1- yl)methyl)phenyl)urea |
| Id-54 | 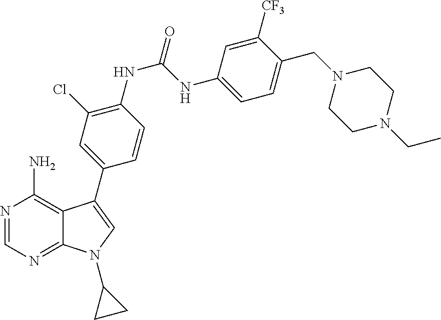 [SMILES: CCN1CCN(Cc2ccc(NC(=O)Nc3ccc(-c4cn(C5CC5)c5ncnc(N)c45)cc3Cl)cc2C(F)(F)F)CC1] | 1-(4-(4-amino-7- cyclopropyl-7H- pyrrolo[2,3- d]pyrimidin-5-yl)-2- chlorophenyl)-3-(4-((4- ethylpiperazin-1- yl)methyl)-3- (trifluoromethyl) phenyl)urea |
| Id-55 | 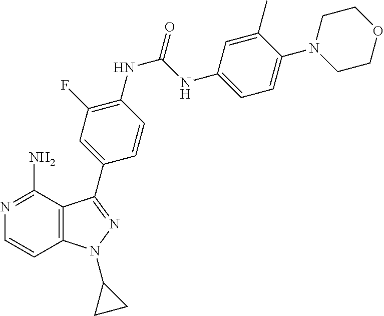 [SMILES: Cc1cc(NC(=O)Nc2ccc(-c3nn(C4CC4)c4ccnc(N)c34)cc2F)ccc1N1CCOCC1] | 1-(4-(4-amino-1- cyclopropyl-1H- pyrazolo[4,3-c] pyridin-3-yl)-2- fluorophenyl)-3-(3- methyl-4-morpholino- phenyl)urea |
| Id-56 | 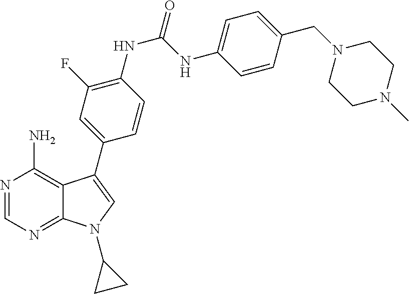 [SMILES: CN1CCN(Cc2ccc(NC(=O)Nc3ccc(-c4cn(C5CC5)c5ncnc(N)c45)cc3F)cc2)CC1] | 1-(4-(4-amino-7- cyclopropyl-7H- pyrrolo[2,3- d]pyrimidin)-5-yl)-2- fluorophenyl)-3-(4-((4- methylpiperazin-1- yl)methyl)phenyl)urea |
| Id-57 | 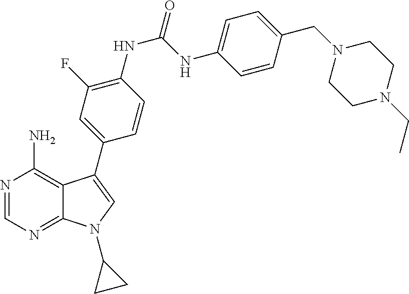 [SMILES: CCN1CCN(Cc2ccc(NC(=O)Nc3ccc(-c4cn(C5CC5)c5ncnc(N)c45)cc3F)cc2)CC1] | 1-(4-(4-amino-7- cyclopropyl-7H- pyrrolo[2,3- d]pyrimidin)-5-yl)-2- fluorophenyl)-3-(4-((4- ethylpiperazin-1- yl)methyl)phenyl)urea |
| Id-58 | 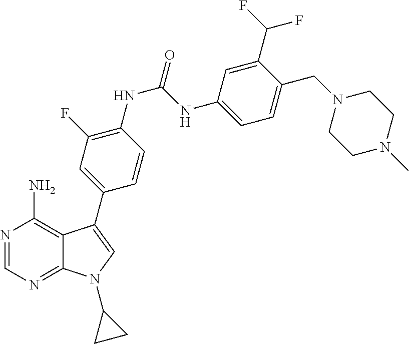 [SMILES: CN1CCN(Cc2ccc(NC(=O)Nc3ccc(-c4cn(C5CC5)c5ncnc(N)c45)cc3F)cc2C(F)F)CC1] | 1-(4-(4-amino-7- cyclopropyl-7H- pyrrolo[2,3- d]pyrimidin)-5-yl)-2- fluorophenyl)-3-(3- (difluoromethyl)-4-((4- methylpiperazin-1- yl)methyl)phenyl)urea |
| Id-59 | 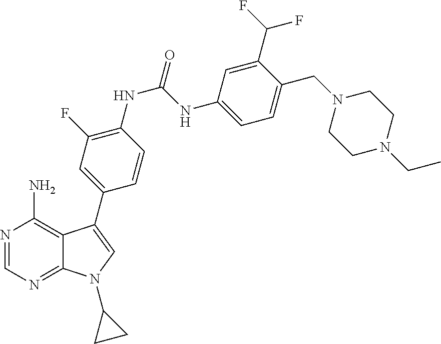 [SMILES: CCN1CCN(Cc2ccc(NC(=O)Nc3ccc(-c4cn(C5CC5)c5ncnc(N)c45)cc3F)cc2C(F)F)CC1] | 1-(4-(4-amino-7- cyclopropyl-7H- pyrrolo[2,3- d]pyrimidin)-5-yl)-2- fluorophenyl)-3-(3- (difluoromethyl)-4-((4- ethylpiperazin-1- yl)methyl)phenyl)urea |
| Id-60 | 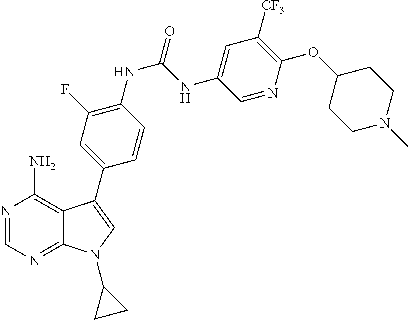 [SMILES: CN1CCC(Oc2ncc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin)-5-yl)-2- fluorophenyl)-3-(6-((1- methylpiperidin-4- yl)oxy)-5- (trifluoromethyl) pyridin-3-yl)urea |
| Id-61 | 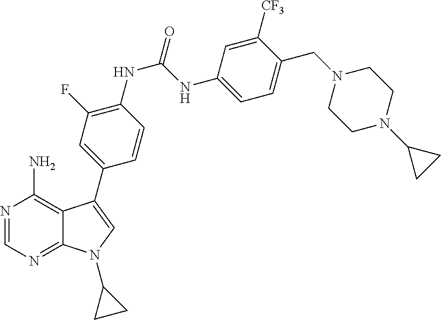 [SMILES: Nc1ncnc2c1c(-c1ccc(NC(=O)Nc3ccc(CN4CCN(C5CC5)CC4)c(C(F)(F)F)c3)c(F)c1)cn2C1CC1] | 1-(4-(4-amino-7- cyclopropyl-7H- pyrrolo[2,3- d]pyrimidin)-5-yl)-2- fluorophenyl)-3-(4-((4- cyclopropylpiperazin-1- yl)methyl)-3- (trifluoromethyl) phenyl)urea |
| Id-62 | 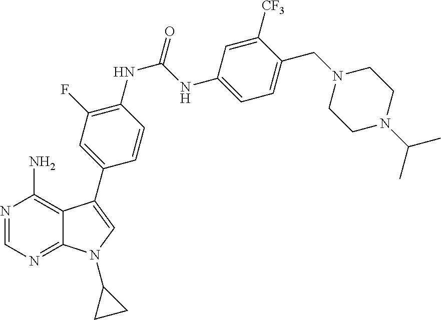 [SMILES: CC(C)N1CCN(Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin)-5-yl)-2- fluorophenyl)-3-(4-((4- isopropylpiperazin-1- yl)methyl)-3- (trifluoromethyl) phenyl)urea |
| Id-63 | 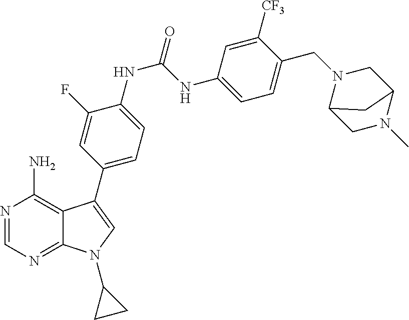 [SMILES: CN1CC2CC1CN2Cc1ccc(NC(=O)Nc2ccc(-c3cn(C4CC4)c4ncnc(N)c34)cc2F)cc1C(F)(F)F] | 1-(4-(4-amino-7- cyclopropyl-7H- pyrrolo[2,3- d]pyrimidin)-5-yl)-2- fluorophenyl)-3-(4-((5- methyl-2,5- diazabicyclo[2.2.1]heptan- 2-yl)methyl)-3- (trifluoromethyl) phenyl)urea |
| Id-64 | 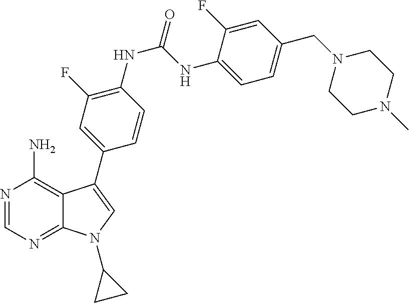 [SMILES: CN1CCN(Cc2ccc(NC(=O)Nc3ccc(-c4cn(C5CC5)c5ncnc(N)c45)cc3F)c(F)c2)CC1] | 1-(4-(4-amino-7- cyclopropyl-7H- pyrrolo[2,3- d]pyrimidin)-5-yl)-2- fluorophenyl)-3-(2- fluoro-4-((4- methylpiperazin-1- yl)methyl)phenyl)urea |
| Id-65 | 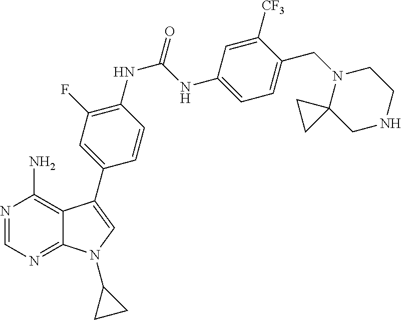 [SMILES: Nc1ncnc2c1c(-c1ccc(NC(=O)Nc3ccc(CN4CCNCC45CC5)c(C(F)(F)F)c3)c(F)c1)cn2C1CC1] | 1-(4-((4,7- diazaspiro[2,5]octan-4- yl)methyl)-3- (trifluoromethyl)phenyl)- 3-(4-(4-amino-7- cyclopropyl-7H- pyrrolo[2,3- d]pyrimidin-5-yl)-2- fluorophenyl)urea |
| Id-66 | 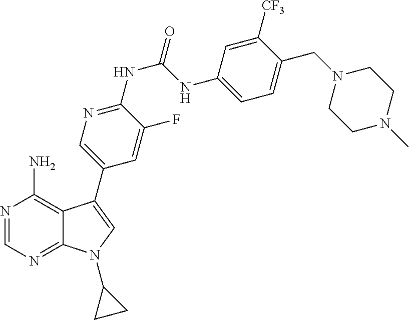 [SMILES: CN1CCN(Cc2ccc(NC(=O)Nc3ncc(-c4cn(C5CC5)c5ncnc(N)c45)cc3F)cc2C(F)(F)F)CC1] | 1-(5-(4-amino-7- cyclopropyl-7H- pyrrolo[2,3- d]pyrimidin)-5-yl)-3- fluorophenyl)-2-yl)-3- (4-((4-methylpiperazin- 1-yl)methyl)-3- (trifluoromethyl) phenyl)urea |
| Id-67 | 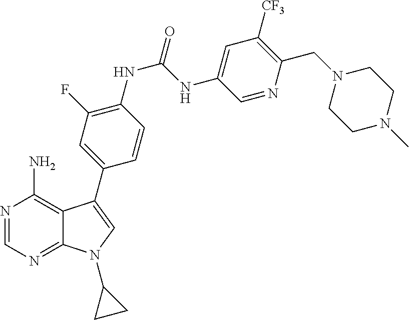 [SMILES: CN1CCN(Cc2ncc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin)-5-yl)-2- fluorophenyl)-3-(6-((4- methylpiperazin-1- yl)methyl)-5- (trifluoromethyl) pyridin-3-yl)urea |
| Id-68 | 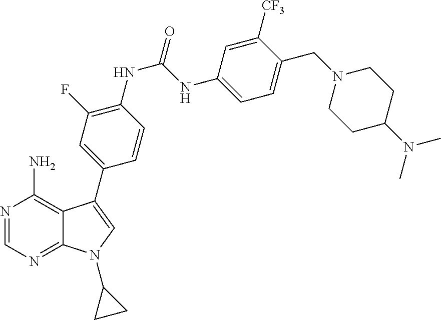 [SMILES: CN(C)C1CCN(Cc2ccc(NC(=O)Nc3ccc(-c4cn(C5CC5)c5ncnc(N)c45)cc3F)cc2C(F)(F)F)CC1] | 1-(4-(4-amino-7- cyclopropyl-7H- pyrrolo[2,3- d]pyrimidin)-5-yl)-2- fluorophenyl)-3-(4-((4- (dimethylamino) piperidin-1-yl)methyl)-3- (trifluoromethyl) phenyl)urea |
| Id-69 | 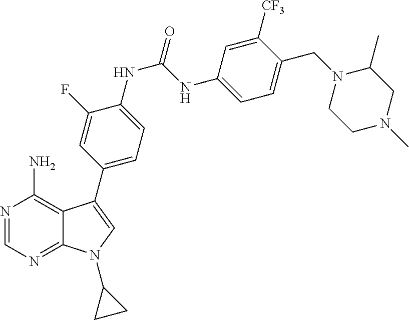 [SMILES: CC1CN(C)CCN1Cc1ccc(NC(=O)Nc2ccc(-c3cn(C4CC4)c4ncnc(N)c34)cc2F)cc1C(F)(F)F] | 1-(4-(4-amino-7- cyclopropyl-7H- pyrrolo[2,3- d]pyrimidin)-5-yl)-2- fluorophenyl)-3-(4- ((2,4-dimethylpiperazin- 1-yl)methyl)-3- (trifluoromethyl) phenyl)urea |
| Id-70 | 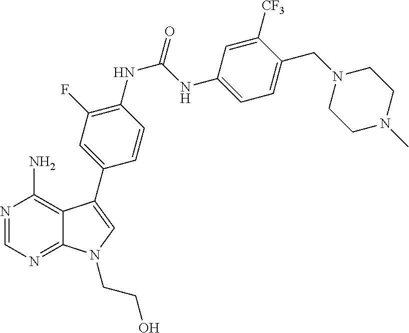 [SMILES: CN1CCN(Cc2ccc(NC(=O)Nc3ccc(-c4cn(CCO)c5ncnc(N)c45)cc3F)cc2C(F)(F)F)CC1] | 1-(4-(4-amino-7-(2- hydroxyethyl)-7H- pyrrolo[2,3- d]pyrimidin)-5-yl)-2- fluorophenyl)-3-(4-((4- methylpiperazin-1- yl)methyl)-3- (trifluoromethyl) phenyl)urea |
| Id-71 | 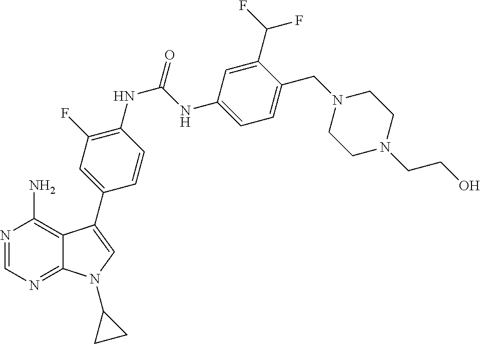 [SMILES: Nc1ncnc2c1c(-c1ccc(NC(=O)Nc3ccc(CN4CCN(CCO)CC4)c(C(F)F)c3)c(F)c1)cn2C1CC1] | 1-(4-(4-amino-7- cyclopropyl-7H- pyrrolo[2,3- d]pyrimidin)-5-yl)-2- fluorophenyl)-3-(3- (difluoromethyl)-4- ((4-(2-hydroxyethyl) piperazin-1-yl) methyl)phenyl)urea |

In some embodiments, the NEK7 small molecule inhibitor compound has the following Structure (Ie):

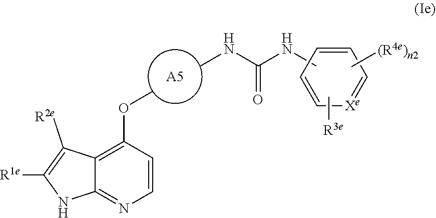

or a pharmaceutically acceptable salt, stereoisomer, or prodrug thereof, wherein:

X^{c} is N or CH;

A5 is C₆-C₁₀ arylene, C₃-C₁₀ cycloalkylene, 3-10 membered heterocyclylene, or 5-6 membered heteroarylene;

R^{1e} is H, halo, C₁-C₆ alkyl, C₃-C₁₀ cycloalkyl, or 3-10 membered heterocyclyl;

R^{2e} is H, halo, C₁-C₆ alkyl, C₁-C₆ haloalkyl, C₃-C₁₀ cycloalkyl, or 3-10 membered heterocyclyl;

R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;

R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and

n2 is 0, 1, 2, 3, or 4.

In some embodiments, A5 is C₁-C₁₀ arylene. In certain embodiments, A5 is phenylene. In some specific embodiments, A5 is 5-6 membered heteroarylene. In certain specific embodiments, A5 is pyridiylene. In some more specific embodiments, A5 is C₃-C₁₀ cycloalkylene or 3-10 membered heterocyclylene.

In certain embodiments, A5 is substituted with one or more substituents selected from halo, cyano, C₁-C₆ alkyl, C₁-C₆ haloalkyl, C₁-C₆ alkoxy, C₁-C₆ haloalkoxy, or C₃-C₈ cycloalkyl. In some embodiments, A5 is substituted with one or more halo substituents. In some specific embodiments, A5 is unsubstituted.

In some embodiments, R^{1e} is H. In certain embodiments, R^{1e} is C₁-C₆ alkyl. In some specific embodiments, R^{1e} is methyl. In certain embodiments, R^{1e} is C₃-C₁₀ cycloalkyl, or 3-10 membered heterocyclyl. In some embodiments, R^{1e} is halo (e.g., fluoro, chloro, bromo, etc.).

In certain embodiments, R^{2e} is H. In some more specific embodiments, R^{2e} is halo. In more specific embodiments, R^{2e} is chloro or fluoro. In some other embodiments, R^{2e} is C₁-C₆ cycloalkyl (e.g., cyclopropyl). In some embodiments, R² is C₁-C₆ alkyl, C₁-C₆ haloalkyl, or 3-10 membered heterocyclyl. In some embodiments, R^{2e} is C₁-C₆ alkyl (e.g., methyl).

In some embodiments, X^{e} is CH. In some specific embodiments, the compound has the following Structure (Ie-Ia):

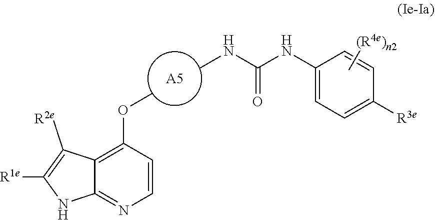

or a pharmaceutically acceptable salt, stereoisomer, or prodrug thereof.

In some embodiments, the compound has the following structure (Ie-Ib):

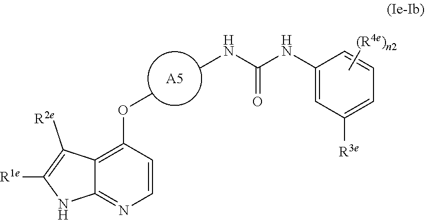

or a pharmaceutically acceptable salt, stereoisomer, or prodrug thereof.

In certain embodiments, X^{e} is N. In certain more specific embodiments, the compound has the following structure (Ie-Ic):

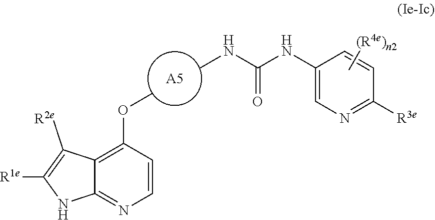

or a pharmaceutically acceptable salt, stereoisomer, or prodrug thereof.

In some embodiments, R^{3e} is substituted. In some embodiments, R^{3e} is unsubstituted. In some embodiments, R^{3e} is substituted with alkyl (e.g., C₁-C₆ alkyl), heterocyclyl, halo, haloalkyl (e.g., C₁-C₆ haloalkyl), alkylcarbonyl (e.g., —C(═O)CH₃), hydroxyalkyl, alkoxyalkyl, cycloalkyl (e.g., C₃-C₈ cycloalkyl), alkylamino, or combinations thereof.

In some of the above embodiments, R^{3e} is aminylalkyl. In certain embodiments, R^{3e} has the following structure:

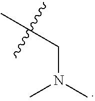

In some embodiments, R^{3e} is a 3-10 membered heterocyclyl. In some embodiments, R^{3e} is morpholino. In certain embodiments, R^{3e} is piperazinyl. In some specific embodiments, R^{3e} has one of the following structures:

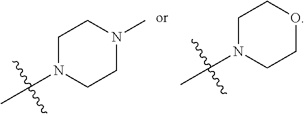

In certain embodiments, R^{3e} is a 3-10 membered heterocyclylalkyl. In certain more specific embodiments, R^{3e} has one of the following structures:

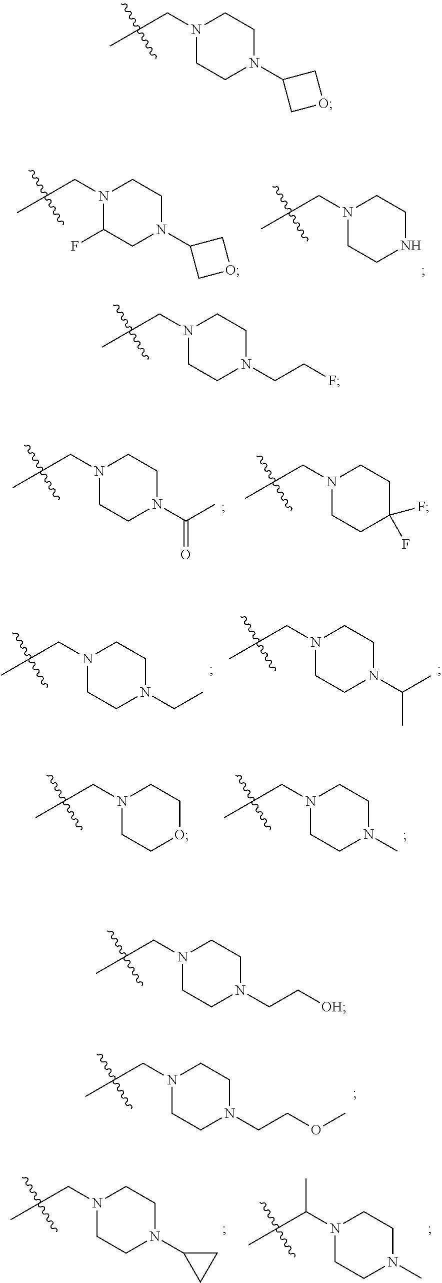

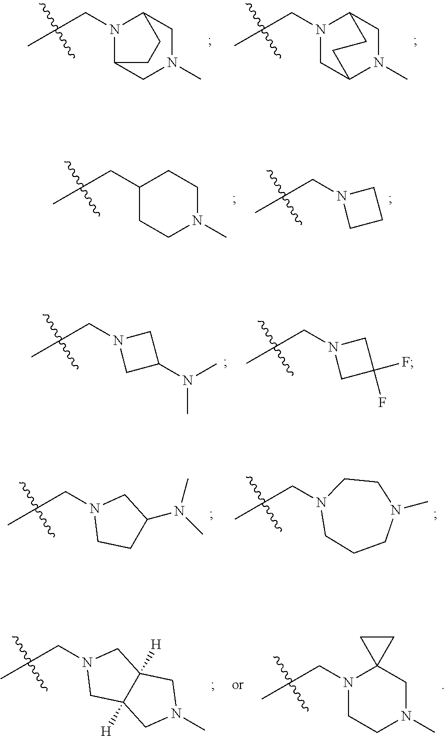

In some embodiments, R^{3e} is 3-10 membered heterocyclylcarbonyl. In a more specific embodiment R^{3e} has the following structure

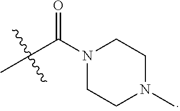 [SMILES: *C(=O)N1CCN(C)CC1]

In some embodiments, R^{3e} is a 5-6 membered heteroaryl. For example, in some embodiments, R^{3e} has the following structure:

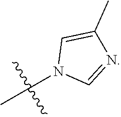 [SMILES: *n1cnc(C)c1]

In some embodiments, R^{3e} is a 3-10 membered heterocyclylalkenyl. In more specific embodiments, R^{3e} has the following structure:

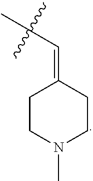 [SMILES: *C=C1CCN(C)CC1]

In some embodiments, R^{3e} is a 3-10 membered N-heterocyclyloxy. In more specific embodiments, R^{3e} has one of the following structures:

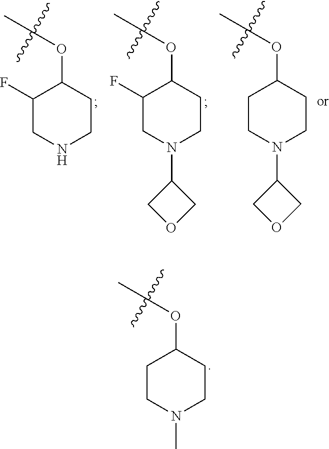

In some embodiments, n2 is 1 or 2. In some embodiments, n2 is 2. In some other embodiments, n2 is 1. In some specific embodiments, R^{4e} is, at each occurrence, independently chloro, fluoro, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl. In certain embodiments, R^{4e} is, at each occurrence, independently methyl, chloro, fluoro, cyano, trifluoromethyl, methoxy, trifluoromethoxy, 2,2-difluorocyclopropyl, or cyclopropyl.

In some embodiments, X^{e} is N and n2 is 3. In some embodiments, X^{e} is N and n2 is 2. In more specific embodiments, X^{e} is N and n2 is 1. In some other embodiments, X^{e} is N and n2 is 0.

In some embodiments, X^{e} is CH and n2 is 4. In some other embodiments, X^{e} is CH and n2 is 3. In some embodiments, X^{e} is CH and n2 is 2. In more specific embodiments, X^{e} is CH and n2 is 1. In some other embodiments, X^{e} is CH and n2 is 0. In various different embodiments, the compound has one of the structures set forth in Table 1e below, or a pharmaceutically acceptable salt, stereoisomer, or prodrug thereof. Compounds in Table 1e were prepared as described in the Examples or methods known in the art and analyzed by mass spectrometry and/or ¹H NMR.

TABLE 1e
Representative compounds of Structure (Ie)

| Cmp | Structure | Name |
|---|---|---|
| Ie-1 | 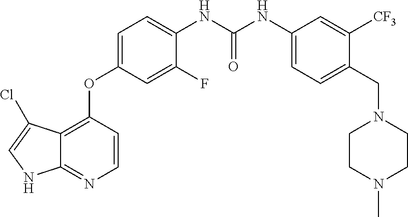 [SMILES: CN1CCN(Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((4-methylpiperazin- 1-yl)methyl)-3- (trifluoromethyl) phenyl)urea |
| Ie-2 | 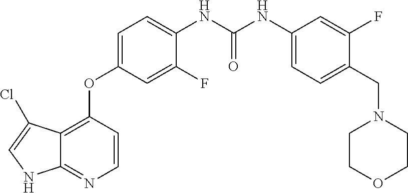 [SMILES: O=C(Nc1ccc(CN2CCOCC2)c(F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(3- fluoro-4- (morpholinomethyl) phenyl)urea |
| Ie-3 | 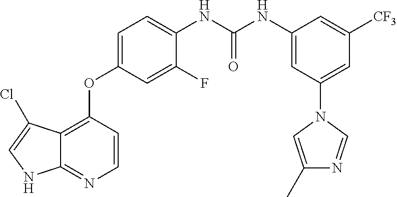 [SMILES: Cc1cn(-c2cc(NC(=O)Nc3ccc(Oc4ccnc5[nH]cc(Cl)c45)cc3F)cc(C(F)(F)F)c2)cn1] | 1-(4-((3-chloro-1H- pyrrolo[2,3- b]pyridin-4-yl)oxy)- 2-fluorophenyl)-3-(3- (4-methyl-1H- imidazol-1-yl)-5- (trifluoromethyl) phenyl)urea |
| Ie-4 | 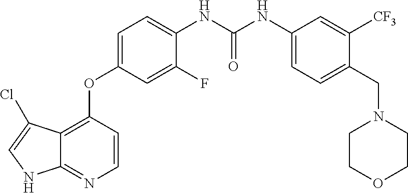 [SMILES: O=C(Nc1ccc(CN2CCOCC2)c(C(F)(F)F)c1)Nc1ccc(Oc2ccnc3[nH]cc(Cl)c23)cc1F] | 1-(4-(3-chloro-1H- pyrrolo[2,3- b]pyridin-4-yl)oxy)- 2-fluorophenyl)-3-(4- (morpholinmethyl)- 3-(trifluoromethyl) phenyl)urea |
| Ie-5 | 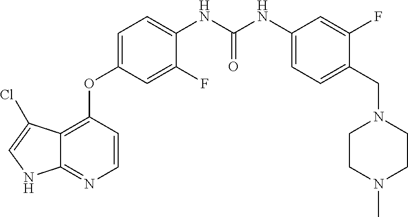 [SMILES: CN1CCN(Cc2ccc(NC(=O)Nc3ccc(Oc4ccnc5[nH]cc(Cl)c45)cc3F)cc2F)CC1] | 1-(4-(3-chloro-1H- pyrrolo[2,3- b]pyridin-4-yl)oxy)- 2-fluorophenyl)-3-(3- fluoro-4-((4- methylpiperazin-1- yl)methyl)phenyl)urea |
| Ie-6 | 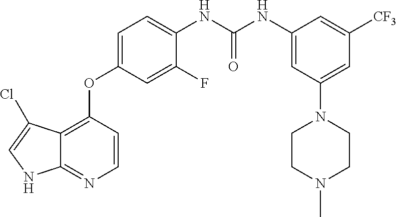 [SMILES: CN1CCN(c2cc(NC(=O)Nc3ccc(Oc4ccnc5[nH]cc(Cl)c45)cc3F)cc(C(F)(F)F)c2)CC1] | 1-(4-((3-chloro-1H- pyrrolo[2,3- b]pyridin-4-yl)oxy)- 2-fluorophenyl)-3-(3- (4-methylpiperazin- 1-yl)-5- (trifluoromethyl) phenyl)urea |
| Ie-7 | 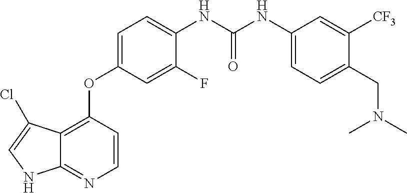 [SMILES: CN(C)Cc1ccc(NC(=O)Nc2ccc(Oc3ccnc4[nH]cc(Cl)c34)cc2F)cc1C(F)(F)F] | 1-(4-((3-chloro-1H- pyrrolo[2,3- b]pyridin-4-yl)oxy)- 2-fluorophenyl)-3-(4- ((dimethylamino) methyl)-3- (trifluoromethyl) phenyl)urea |
| Ie-8 | 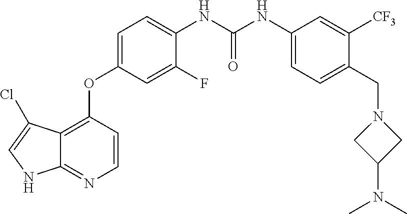 [SMILES: CN(C)C1CN(Cc2ccc(NC(=O)Nc3ccc(Oc4ccnc5[nH]cc(Cl)c45)cc3F)cc2C(F)(F)F)C1] | 1-(4-((3-chloro-1H- pyrrolo[2,3- b]pyridin-4-yl)oxy)- 2-fluorophenyl)-3-(4- ((3-(dimethylamino) azetidin-1-yl)methyl)- 3-(trifluoromethyl) phenyl)urea |
| Ie-9 | 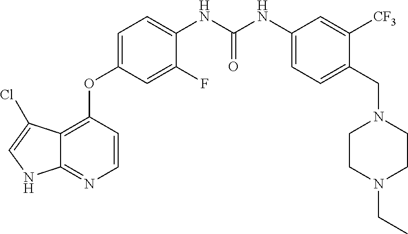 [SMILES: CCN1CCN(Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((4-ethylpiperazin-1- yl)methyl)-3- (trifluoromethyl) phenyl)urea |
| Ie-10 | 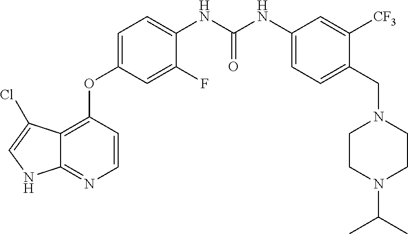 [SMILES: CC(C)N1CCN(Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((4-isopropyl- piperazin-1- yl)methyl)-3- (trifluoromethyl) phenyl)urea |
| Ie-11 | 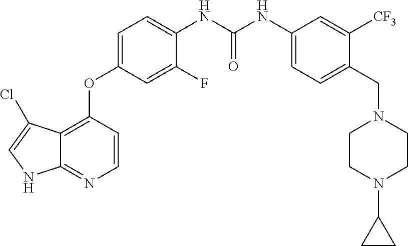 [SMILES: O=C(Nc1ccc(CN2CCN(C3CC3)CC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((4-cyclopropylpiperazin- 1-yl)methyl)-3- (trifluoromethyl) phenyl)urea |
| Ie-12 | 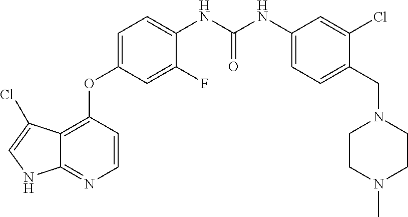 [SMILES: CN1CCN(Cc2ccc(NC(=O)Nc3ccc(Oc4ccnc5[nH]cc(Cl)c45)cc3F)cc2Cl)CC1] | 1-(4-((3-chloro-1H- pyrrolo[2,3- b]pyridin-4-yl)oxy)- 2-fluorophenyl)-3-(3- chloro-4-((4- methylpiperazin-1- yl)methyl)phenyl)urea |
| Ie-13 | 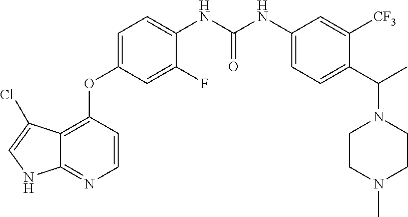 [SMILES: CC(c1ccc(NC(=O)Nc2ccc(Oc3ccnc4[nH]cc(Cl)c34)cc2F)cc1C(F)(F)F)N1CCN(C)CC1] | 1-(4-((3-chloro-1H- pyrrolo[2,3- b]pyridin-4-yl)oxy)- 2-fluorophenyl)-3-(4- (1-(4- methylpiperazin-1- yl)ethyl)-3- (trifluoromethyl) phenyl)urea |
| Ie-14 | 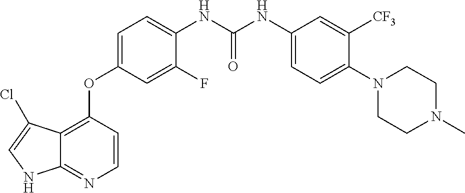 [SMILES: CN1CCN(c2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- (4-methylpiperazin- 1-yl)-3- (trifluoromethyl) phenyl)urea |
| Ie-15 | 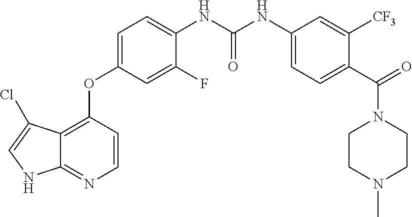 [SMILES: CN1CCN(C(=O)c2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- (4-methylpiperazine- 1-carbonyl)-3- (trifluoromethyl) phenyl)urea |
| Ie-16 | 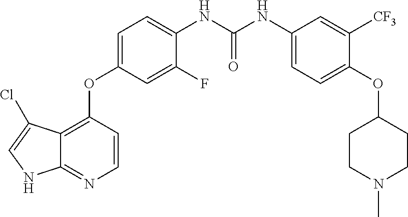 [SMILES: CN1CCC(Oc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pynidin-4-yl)oxy)- 2-fluorophenyl)-3-(4- ((1-methylpiperidin- 4-yl)oxy)-3- (trifluoromethyl) phenyl)urea |
| Ie-17 | 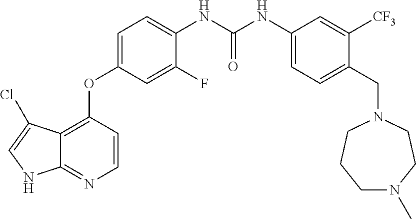 [SMILES: CN1CCCN(Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((4-methyl-1,4- diazepan-1- yl)methyl)-3- (trifluoromethyl) phenyl)urea |
| Ie-18 | 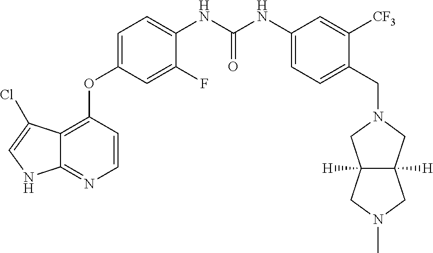 [SMILES: CN1C[C@@H]2CN(Cc3ccc(NC(=O)Nc4ccc(Oc5ccnc6[nH]cc(Cl)c56)cc4F)cc3C(F)(F)F)C[C@@H]2C1] | 1-(4-((3-chloro-1H- pyrrolo[2,3- b]pyridin-4-yl)oxy)- 2-fluorophenyl)-3-(4- (((3aR,6aS)-5- methylhexahydro- pyrrolo[3,4-c]pyrrol- 2(1H)-yl)methyl)-3- (trifluoromethyl) phenyl)urea |
| Ie-19 | 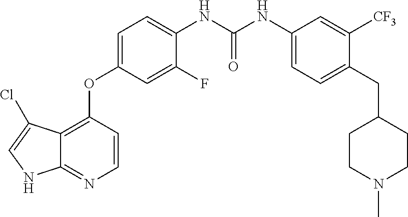 [SMILES: CN1CCC(Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((1-methylpiperidin- 4-yl)methyl)-3- (trifluoromethyl) phenyl)urea |
| Ie-20 | 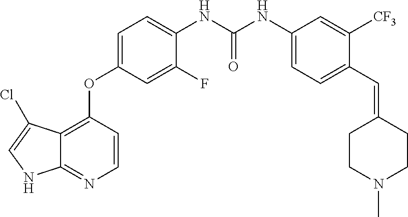 [SMILES: CN1CCC(=Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((1-methylpiperidin- 4-ylidene)methyl)-3- (trifluoromethyl) phenyl)urea |
| Ie-21 | 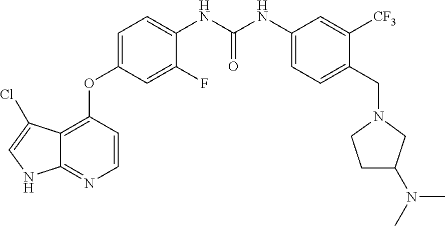 [SMILES: CN(C)C1CCN(Cc2ccc(NC(=O)Nc3ccc(Oc4ccnc5[nH]cc(Cl)c45)cc3F)cc2C(F)(F)F)C1] | 1-(4-((3-chloro-1H- pyrrolo[2,3- b]pyridin-4-yl)oxy)- 2-fluorophenyl)-3-(4- ((3-(dimethylamino) pyrrolidin-1-yl) methyl)-3- (trifluoromethyl) phenyl)urea |
| Ie-22 | 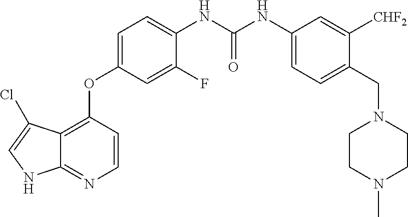 [SMILES: CN1CCN(Cc2ccc(NC(=O)Nc3ccc(Oc4ccnc5[nH]cc(Cl)c45)cc3F)cc2C(F)F)CC1] | 1-(4-((3-chloro-1H- pyrrolo[2,3- b]pyridin-4-yl)oxy)- 2-fluorophenyl)-3-(3- (difluoromethyl)-4- ((4-methylpiperazin- 1-yl)methyl) phenyl)urea |
| Ie-23 | 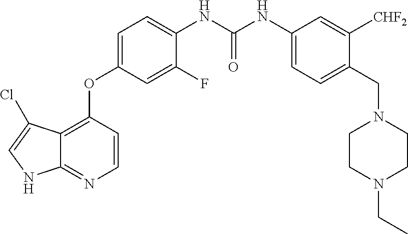 [SMILES: CCN1CCN(Cc2ccc(NC(=O)Nc3ccc(Oc4ccnc5[nH]cc(Cl)c45)cc3F)cc2C(F)F)CC1] | 1-(4-((3-chloro-1H- pyrrolo[2,3- b]pyridin-4-yl)oxy)- 2-fluorophenyl)-3-(3- (difluoromethyl)-4- ((4-ethylpiperazin-1- yl)methyl) phenyl)urea |
| Ie-24 | 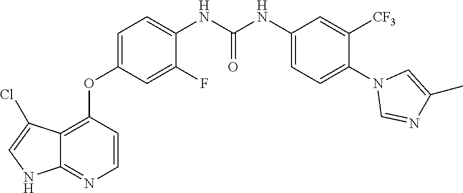 [SMILES: Cc1cn(-c2ccc(NC(=O)Nc3ccc(Oc4ccnc5[nH]cc(Cl)c45)cc3F)cc2C(F)(F)F)cn1] | 1-(4-((3-chloro-1H- pyrrolo[2,3- b]pynidin-4-yl)oxy)- 2-fluorophenyl)-3-(4- (4-methyl-1H- imidazol-1-yl)-3- (trifluoromethyl) phenyl)urea |
| Ie-25 | 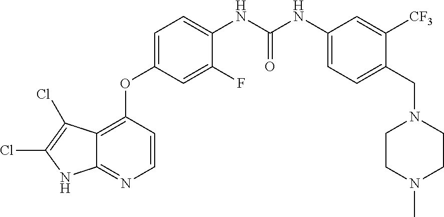 [SMILES: CN1CCN(Cc2ccc(NC(=O)Nc3ccc(Oc4ccnc5[nH]c(Cl)c(Cl)c45)cc3F)cc2C(F)(F)F)CC1] | 1-(4-((2,3-dichloro- 1H-pyrrolo[2,3- b]pyridin-4-yl)oxy)- 2-fluorophenyl)-3-(4- ((4-methylpiperazin- 1-yl)methyl)-3- (trifluoromethyl) phenyl)urea |
| Ie-26 | 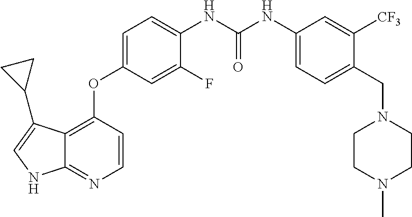 [SMILES: CN1CCN(Cc2ccc(NC(=O)Nc3ccc(Oc4ccnc5[nH]cc(C6CC6)c45)cc3F)cc2C(F)(F)F)CC1] | 1-(4-((3-cyclopropyl- 1H-pyrrolo[2,3- b]pyridin-4-yl)oxy)- 2-fluorophenyl)-3-(4- ((4-methylpiperazin- 1-yl)methyl)-3- (trifluoromethyl) phenyl)urea |
| Ie-27 | 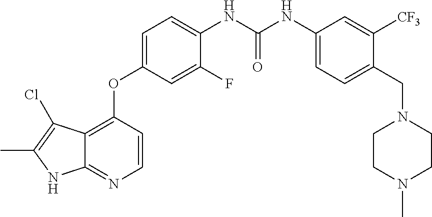 [SMILES: Cc1[nH]c2nccc(Oc3ccc(NC(=O)Nc4ccc(CN5CCN(C)CC5)c(C(F)(F)F)c4)c(F)c3)c2c1Cl] | 1-(4-((3-chloro-2- methyl-1H- pyrrolo[2,3- b]pyridin-4-yl)oxy)- 2-fluorophenyl)-3-(4- ((4-methylpiperazin- 1-yl)methyl)-3- (trifluoromethyl) phenyl)urea |
| Ie-28 | 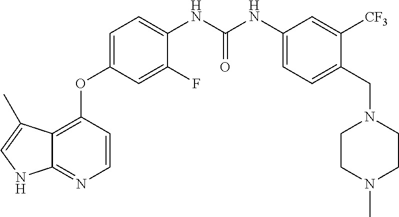 [SMILES: Cc1c[nH]c2nccc(Oc3ccc(NC(=O)Nc4ccc(CN5CCN(C)CC5)c(C(F)(F)F)c4)c(F)c3)c12] | 1-(2-fluoro-4-((3- methyl-1H- pyrrolo[2,3- b]pyridin-4- yl)oxy)phenyl)-3-(4- ((4-methylpiperazin- 1-yl)methyl)-3- (trifluoromethyl) phenyl)urea |
| Ie-29 | 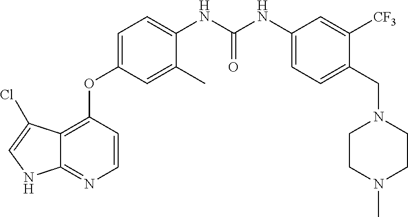 [SMILES: Cc1cc(Oc2ccnc3[nH]cc(Cl)c23)ccc1NC(=O)Nc1ccc(CN2CCN(C)CC2)c(C(F)(F)F)c1] | 1-(4-((3-chloro-1H- pyrrolo[2,3- b]pyridin-4-yl)oxy)- 2-methylphenyl)-3- (4-((4- methylpiperazin-1- yl)methyl)-3- (trifluoromethyl) phenyl)urea |
| Ie-30 | 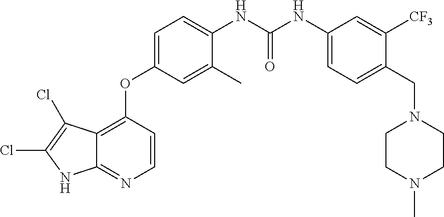 [SMILES: Cc1cc(Oc2ccnc3[nH]c(Cl)c(Cl)c23)ccc1NC(=O)Nc1ccc(CN2CCN(C)CC2)c(C(F)(F)F)c1] | 1-(4-((2,3-dichloro- 1H-pyrrolo[2,3- b]pyridin-4-yl)oxy)- 2-methylphenyl)-3- (4-((4- methylpiperazin-1- yl)methyl)-3- (trifluoromethyl) phenyl)urea |
| Ie-31 | 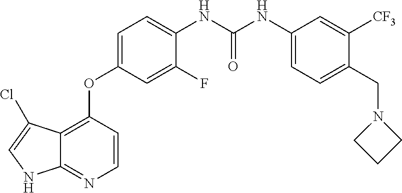 [SMILES: O=C(Nc1ccc(CN2CCC2)c(C(F)(F)F)c1)Nc1ccc(Oc2ccnc3[nH]cc(Cl)c23)cc1F] | 1-(4-(azetidin-1- ylmethyl)-3- (trifluoromethyl) phenyl)-3-(4-((3- chloro-1H-pyrrolo[2,3- b]pyridin-4-yl)oxy)- 2-fluorophenyl)urea |
| Ie-32 | 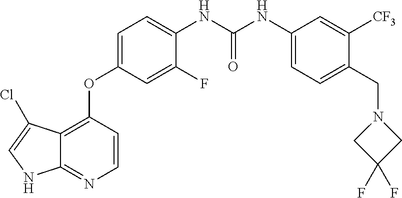 [SMILES: O=C(Nc1ccc(CN2CC(F)(F)C2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((3,3- difluoroazetidin-1- yl)methyl)-3- (trifluoromethyl) phenyl)urea |
| Ie-33 | 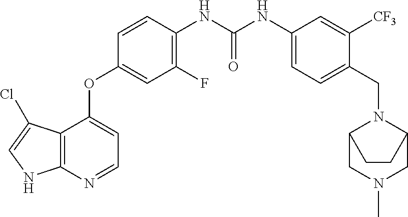 [SMILES: CN1CC2CCC(C1)N2Cc1ccc(NC(=O)Nc2ccc(Oc3ccnc4[nH]cc(Cl)c34)cc2F)cc1C(F)(F)F] | 1-(4-((3-chloro-1H- pyrrolo[2,3- b]pyridin-4-yl)oxy)- 2-fluorophenyl)-3-(4- (((1R,5S)-3-methyl- 3,8-diazabicyclo[3.2.1] octan-8-yl)methyl)-3- (trifluoromethyl) phenyl)urea |
| Ie-34 | 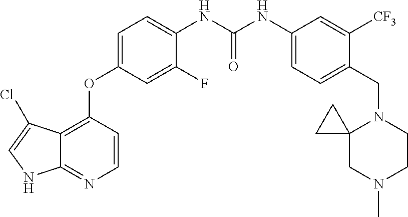 [SMILES: CN1CCN(Cc2ccc(NC(=O)Nc3ccc(Oc4ccnc5[nH]cc(Cl)c45)cc3F)cc2C(F)(F)F)C2(CC2)C1] | 1-(4-((3-chloro-1H- pyrrolo[2,3- b]pyridin-4-yl)oxy)- 2-fluorophenyl)-3-(4- ((7-methyl-4,7- diazaspiro[2.5]octan- 4-yl)methyl)-3- (trifluoromethyl) phenyl)urea |
| Ie-35 | 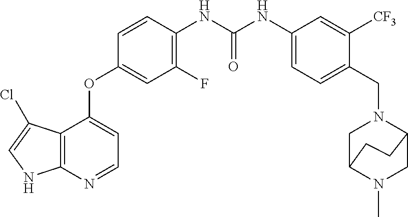 [SMILES: CN1CC2CCC1CN2Cc1ccc(NC(=O)Nc2ccc(Oc3ccnc4[nH]cc(Cl)c34)cc2F)cc1C(F)(F)F] | 1-(4-((3-chloro-1H- pyrrolo[2,3- b]pyridin-4-yl)oxy)- 2-fluorophenyl)-3-(4- ((5-methyl-2,5- diazabicyclo[2.2.2] octan-2-yl)methyl)-3- (trifluoromethyl) phenyl)urea |
| Ie-36 | 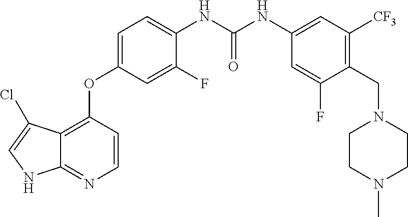 [SMILES: CN1CCN(Cc2c(F)cc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridm-4-yl)oxy)- 2-fluorophenyl)-3-(3- fluoro-4-((4- methylpiperazin-1- yl)methyl)-5- (trifluoromethyl) phenyl)urea |
| Ie-37 | 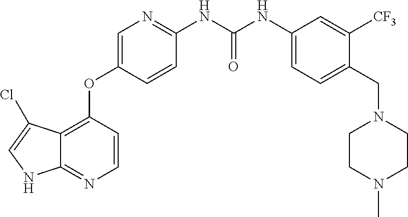 [SMILES: CN1CCN(Cc2ccc(NC(=O)Nc3ccc(Oc4ccnc5[nH]cc(Cl)c45)cn3)cc2C(F)(F)F)CC1] | 1-5-((3-chloro-1H- pyrrolo[2,3- b]pyridin-4- yl)oxy)pyridin-2-yl)- 3-(4-((4- methylpiperazin-1- yl)methyl)-3- (trifluoromethyl) phenyl)urea |
| Ie-38 | 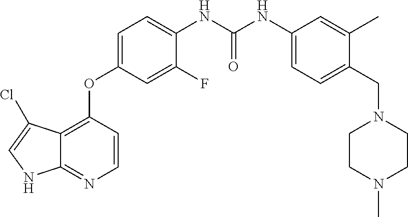 [SMILES: Cc1cc(NC(=O)Nc2ccc(Oc3ccnc4[nH]cc(Cl)c34)cc2F)ccc1CN1CCN(C)CC1] | 1-(4-((3-chloro-1H- pyrrolo[2,3- b]pyridin-4-yl)oxy)- 2-fluorophenyl)-3-(3- methyl-4-((4- methylpiperazin-1- yl)methyl)phenyl)urea |
| Ie-39 | 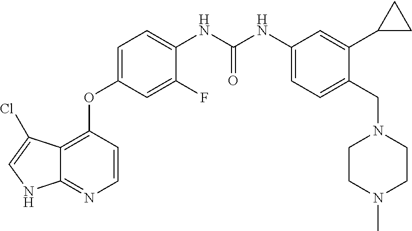 [SMILES: CN1CCN(Cc2ccc(NC(=O)Nc3ccc(Oc4ccnc5[nH]cc(Cl)c45)cc3F)cc2C2CC2)CC1] | 1-(4-((3-chloro-1H- pyrrolo[2,3- b]pyridin-4-yl)oxy)- 2-fluorophenyl)-3-(3- cyclopropyl-4-((4- methylpiperazin-1- yl)methyl)phenyl)urea |
| Ie-40 | 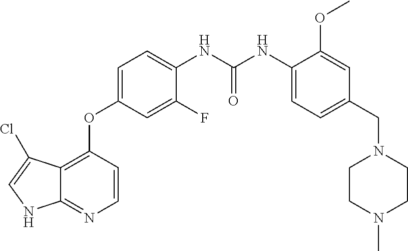 [SMILES: COc1cc(CN2CCN(C)CC2)ccc1NC(=O)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(2- methoxy-4-((4- methylpiperazin-1- yl)methyl)phenyl)urea |
| Ie-41 | 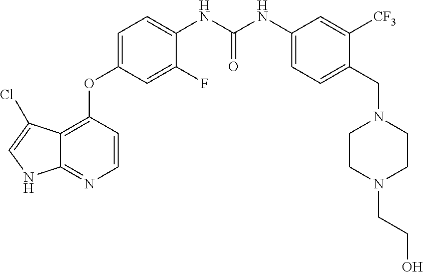 [SMILES: O=C(Nc1ccc(CN2CCN(CCO)CC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((4-(2- hydroxyethyl)piperazin- 1-yl)methyl)-3- (trifluoromethyl) phenyl)urea |
| Ie-42 | 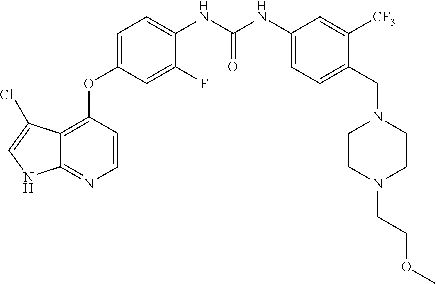 [SMILES: COCCN1CCN(Cc2ccc(NC(=O)Nc3ccc(Oc4ccnc5[nH]cc(Cl)c45)cc3F)cc2C(F)(F)F)CC1] | 1-(4-((3-chloro-1H- pyrrolo[2,3- b]pyridin-4-yl)oxy)- 2-fluorophenyl)-3-(4- ((4-(2- methoxyethyl)piperazin- 1-yl)methyl)-3- (trifluoromethyl) phenyl)urea |
| Ie-43 | 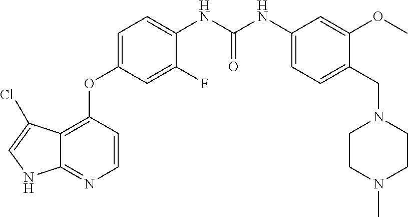 [SMILES: COc1cc(NC(=O)Nc2ccc(Oc3ccnc4[nH]cc(Cl)c34)cc2F)ccc1CN1CCN(C)CC1] | 1-(4-((3-chloro-1H- pyrrolo[2,3- b]pyridin-4-yl)oxy)- 2-fluorophenyl)-3-(3- methoxy-4-((4- methylpiperazin-1- yl)methyl)phenyl)urea |
| Ie-44 | 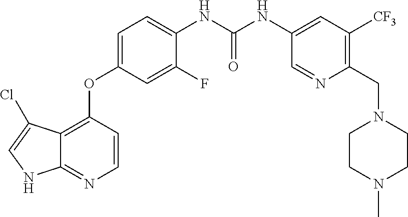 [SMILES: CN1CCN(Cc2ncc(NC(=O)Nc3ccc(Oc4ccnc5[nH]cc(Cl)c45)cc3F)cc2C(F)(F)F)CC1] | 1-(4-(3-chloro-1H- pyrrolo[2,3- b]pyridin-4-yl)oxy)- 2-fluorophenyl)-3-(6- ((4-methylpiperazin- 1-yl)methyl)-5- (trifluoromethyl) pyridin-3-yl)urea |
| Ie-45 | 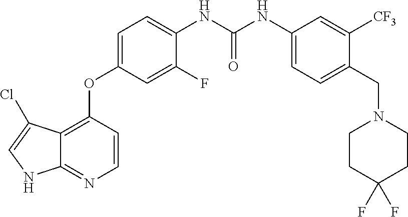 [SMILES: O=C(Nc1ccc(CN2CCC(F)(F)CC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((4,4- difluoropiperidin-1- yl)methyl)-3- (trifluoromethyl) phenyl)urea |
| Ie-46 | 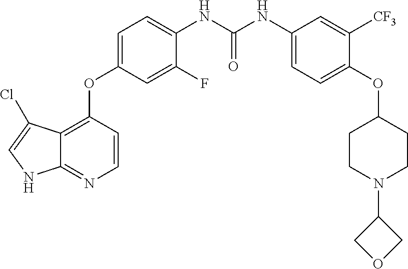 [SMILES: O=C(Nc1ccc(OC2CCN(C3COC3)CC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((1-(oxetan-3- yl)piperidin-4- yl)oxy)-3- (trifluoromethyl) phenyl)urea |
| Ie-47 | 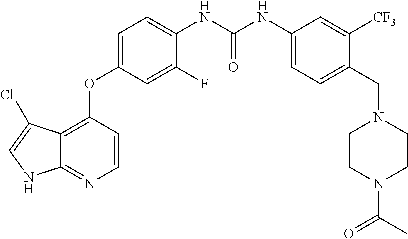 [SMILES: CC(=O)N1CCN(Cc2ccc(NC(=O)Nc3ccc(Oc4ccnc5[nH]cc(Cl)c45)cc3F)cc2C(F)(F)F)CC1] | 1-(4-((4- acetylpiperazin-1- yl)methyl)-3- (trifluoromethyl) phenyl)-3-(4-((3-chloro- 1H-pyrrolo[2,3- b]pyridin-4-yl)oxy)- 2-fluorophenyl)urea |
| Ie-48 | 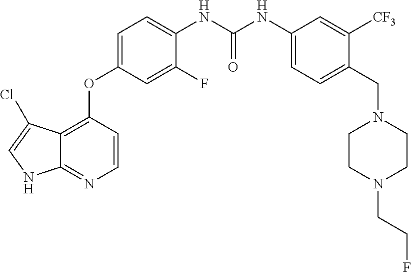 [SMILES: O=C(Nc1ccc(CN2CCN(CCF)CC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((4-(2- fluoroethyl)piperazin- 1-yl)methyl)-3- (trifluoromethyl) phenyl)urea |
| Ie-49 | 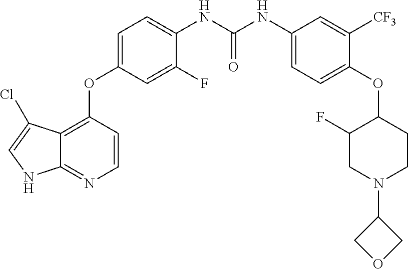 [SMILES: O=C(Nc1ccc(OC2CCN(C3COC3)CC2F)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((3-fluoro-1-(oxetan- 3-yl)piperidin-4- yl)oxy)-3- (trifluoromethyl) phenyl)urea |
| Ie-50 | 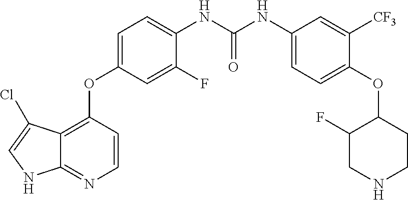 [SMILES: O=C(Nc1ccc(OC2CCNCC2F)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((3-fluoropiperidin- 4-yl)oxy)-3- (trifluoromethyl) phenyl)urea |
| Ie-51 | 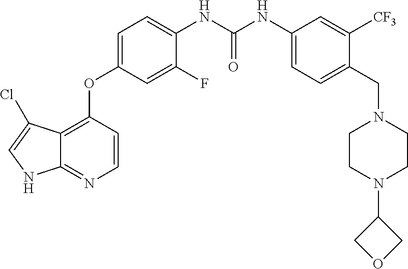 [SMILES: O=C(Nc1ccc(CN2CCN(C3COC3)CC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((4-(oxetan-3- yl)piperazin-1- yl)methyl)-3- (trifluoromethyl) phenyl)urea |
| Ie-52 | 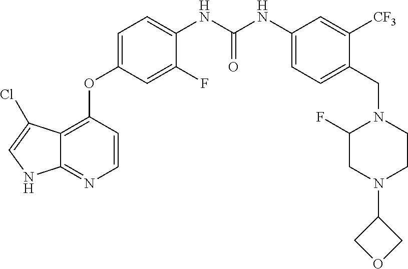 [SMILES: O=C(Nc1ccc(CN2CCN(C3COC3)CC2F)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- ((2-fluoro-4-(oxetan- 3-yl)piperazin-1- yl)methyl)-3- (trifluoromethyl) phenyl)urea |
| Ie-53 | 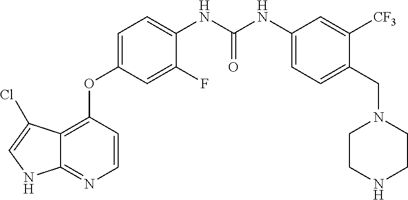 [SMILES: O=C(Nc1ccc(CN2CCNCC2)c(C(F)(F)F)c1)Nc1ccc(Oc2ccnc3[nH]cc(Cl)c23)cc1F] | 1-(4-((3-chloro-1H- pyrrolo[2,3- b]pyridin-4-yl)oxy)- 2-fluorophenyl)-3-(4- (piperazin-1- ylmethyl)-3- (trifluoromethyl) phenyl)urea |
| Ie-54 | 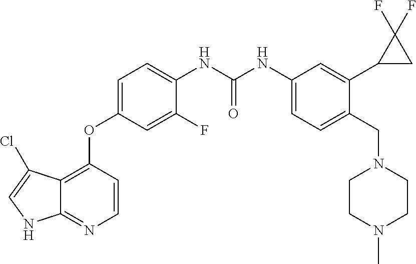 [SMILES: CN1CCN(Cc2ccc(NC(=O)Nc3ccc(Oc4ccnc5[nH]cc(Cl)c45)cc3F)cc2C2CC2(F)F)CC1] | 1-(4-((3-chloro-1H- pyrrolo[2,3- b]pyridin-4-yl)oxy)- 2-fluorophenyl)-3-(3- (2,2-difluorocyclopropyl)- 4-((4- methylpiperazin-1- yl)methyl)phenyl)urea |

In some embodiments, the NEK7 small molecule inhibitor compound is disclosed in PCT Publication No. WO 2019/192962, the entirety of which is hereby incorporated by reference.

Accordingly, in some embodiments, the NEK7 small molecule inhibitor compound has the following Structure (If).

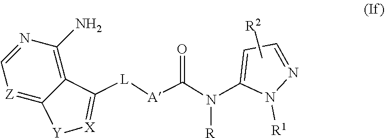

or a pharmaceutically acceptable salt, stereoisomer, or prodrug thereof, wherein:

A′ is C═O, C(R)₂ or NR;

L is a divalent group selected from C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted;

X is CR or N;

Y is NR or S;

Z is CR or N;

R¹ is C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted:

R² is —R, halogen, -haloalkyl, —OR, —SR, —CN, —NO₂, —SO₂R, —SOR, —C(═O)R, —CO₂R,

—C(═O)N(R)₂, —NRC(═O)R₂, —NRC(═O)N(R)₂, —NRSO₂R, or —N(R)₂;

each R is independently hydrogen, C₁₋₆ aliphatic, C₃₋₁₀ aryl, a 3-8 membered saturated or partially unsaturated carbocyclic ring, a 3-7 membered heterocylic ring having 1-4 hetero atoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or

two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted.

In various different embodiments, the compound has one of the structures set forth in Table 1f below, or a pharmaceutically acceptable salt, stereoisomer, or prodrug thereof.

TABLE If
Representative compounds of Structure (If)

| Compound No | Compound Structure |
|---|---|
| If-1 | 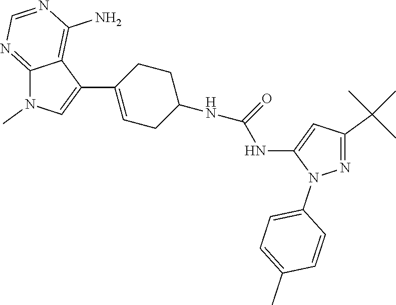 [SMILES: Cc1ccc(-n2nc(C(C)(C)C)cc2NC(=O)NC2CC=C(c3cn(C)c4ncnc(N)c34)CC2)cc1] |
| If-2 | 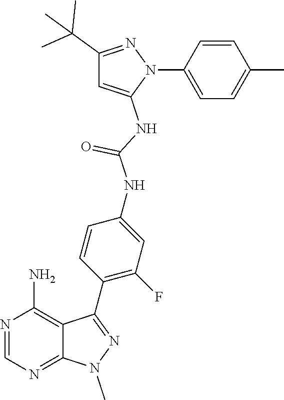 [SMILES: Cc1ccc(-n2nc(C(C)(C)C)cc2NC(=O)Nc2ccc(-c3nn(C)c4ncnc(N)c34)c(F)c2)cc1] |
| If-3 | 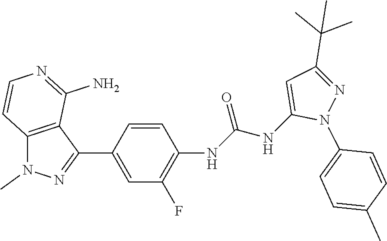 [SMILES: Cc1ccc(-n2nc(C(C)(C)C)cc2NC(=O)Nc2ccc(-c3nn(C)c4ccnc(N)c34)cc2F)cc1] |
| If-4 | 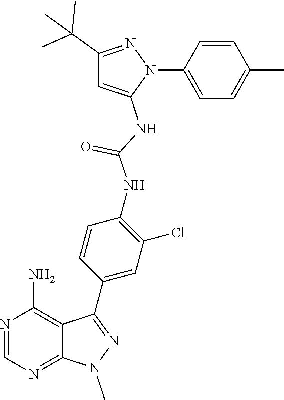 [SMILES: Cc1ccc(-n2nc(C(C)(C)C)cc2NC(=O)Nc2ccc(-c3nn(C)c4ncnc(N)c34)cc2Cl)cc1] |
| If-5 | 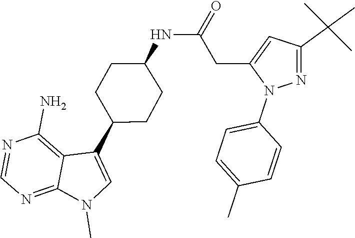 [SMILES: Cc1ccc(-n2nc(C(C)(C)C)cc2CC(=O)N[C@H]2CC[C@@H](c3cn(C)c4ncnc(N)c43)CC2)cc1] |
| If-6 | 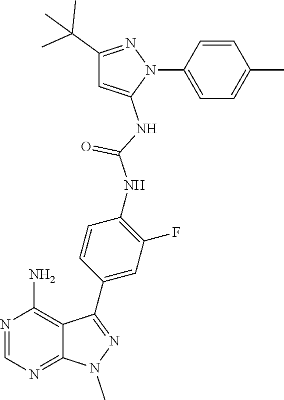 [SMILES: Cc1ccc(-n2nc(C(C)(C)C)cc2NC(=O)Nc2ccc(-c3nn(C)c4ncnc(N)c34)cc2F)cc1] |
| If-7 | 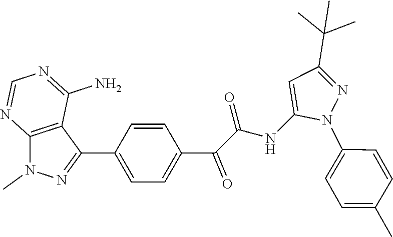 [SMILES: Cc1ccc(-n2nc(C(C)(C)C)cc2NC(=O)C(=O)c2ccc(-c3nn(C)c4ncnc(N)c34)cc2)cc1] |
| If-8 | 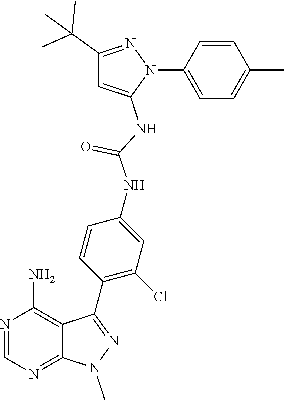 [SMILES: Cc1ccc(-n2nc(C(C)(C)C)cc2NC(=O)Nc2ccc(-c3nn(C)c4ncnc(N)c34)c(Cl)c2)cc1] |
| If-9 | 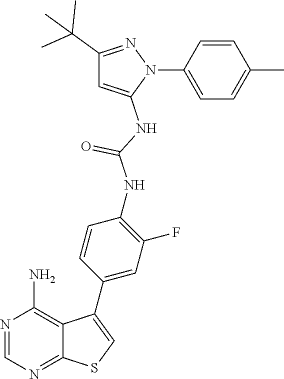 [SMILES: Cc1ccc(-n2nc(C(C)(C)C)cc2NC(=O)Nc2ccc(-c3csc4ncnc(N)c34)cc2F)cc1] |
| If-10 | 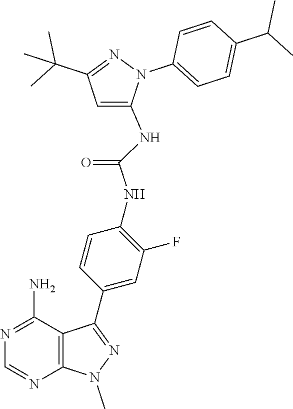 [SMILES: CC(C)c1ccc(-n2nc(C(C)(C)C)cc2NC(=O)Nc2ccc(-c3nn(C)c4ncnc(N)c34)cc2F)cc1] |
| If-11 | 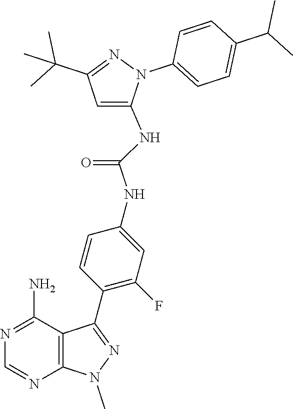 [SMILES: CC(C)c1ccc(-n2nc(C(C)(C)C)cc2NC(=O)Nc2ccc(-c3nn(C)c4ncnc(N)c34)c(F)c2)cc1] |
| If-12 | 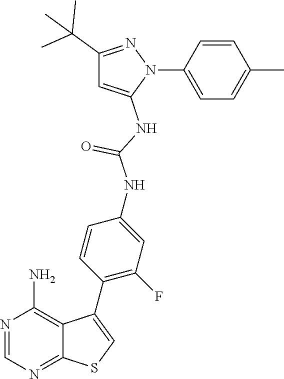 [SMILES: Cc1ccc(-n2nc(C(C)(C)C)cc2NC(=O)Nc2ccc(-c3csc4ncnc(N)c34)c(F)c2)cc1] |
| If-13 | 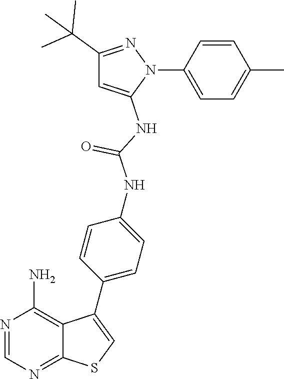 [SMILES: Cc1ccc(-n2nc(C(C)(C)C)cc2NC(=O)Nc2ccc(-c3csc4ncnc(N)c34)cc2)cc1] |
| If-14 | 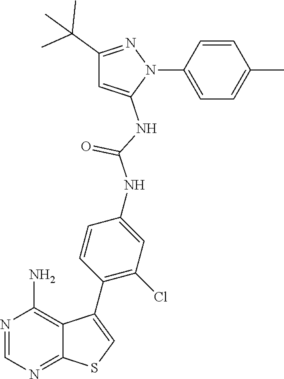 [SMILES: Cc1ccc(-n2nc(C(C)(C)C)cc2NC(=O)Nc2ccc(-c3csc4ncnc(N)c34)c(Cl)c2)cc1] |
| If-15 | 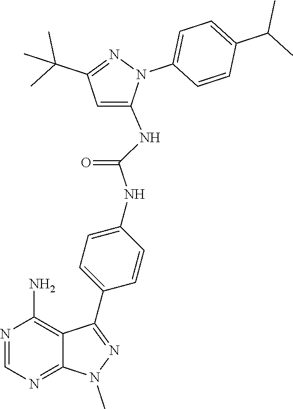 [SMILES: CC(C)c1ccc(-n2nc(C(C)(C)C)cc2NC(=O)Nc2ccc(-c3nn(C)c4ncnc(N)c34)cc2)cc1] |
| If-16 | 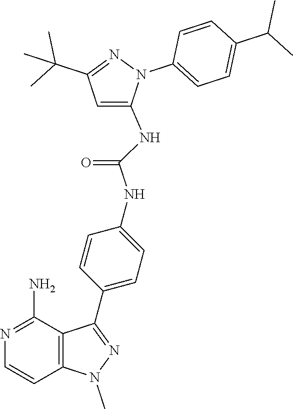 [SMILES: CC(C)c1ccc(-n2nc(C(C)(C)C)cc2NC(=O)Nc2ccc(-c3nn(C)c4ccnc(N)c34)cc2)cc1] |
| If-17 | 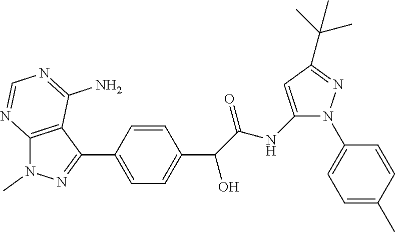 |
| If-18 | 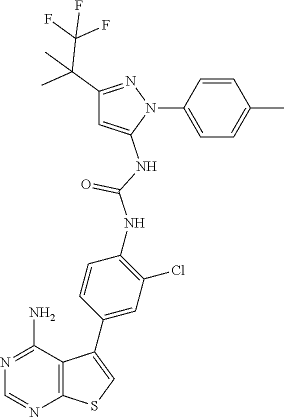 [SMILES: Cc1ccc(-n2nc(C(C)(C)C(F)(F)F)cc2NC(=O)Nc2ccc(-c3csc4ncnc(N)c34)cc2Cl)cc1] |
| If-19 | 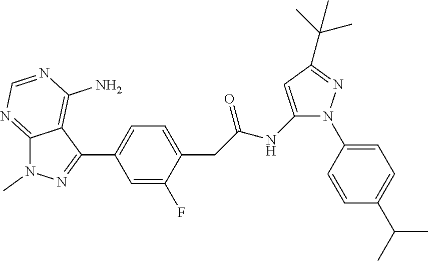 [SMILES: CC(C)c1ccc(-n2nc(C(C)(C)C)cc2NC(=O)Cc2ccc(-c3nn(C)c4ncnc(N)c34)cc2F)cc1] |
| If-20 | 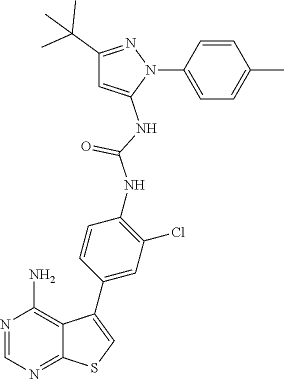 [SMILES: Cc1ccc(-n2nc(C(C)(C)C)cc2NC(=O)Nc2ccc(-c3csc4ncnc(N)c34)cc2Cl)cc1] |
| If-21 | 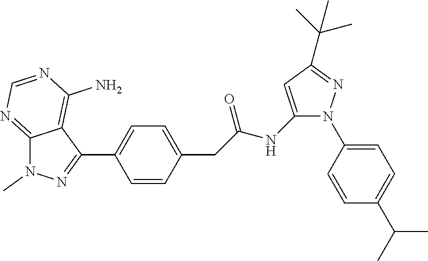 [SMILES: CC(C)c1ccc(-n2nc(C(C)(C)C)cc2NC(=O)Cc2ccc(-c3nn(C)c4ncnc(N)c34)cc2)cc1] |
| If-22 | 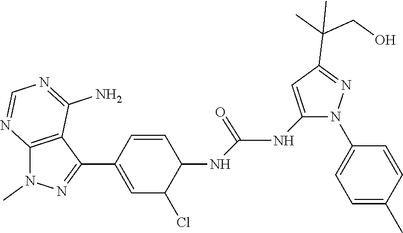 |
| If-23 | 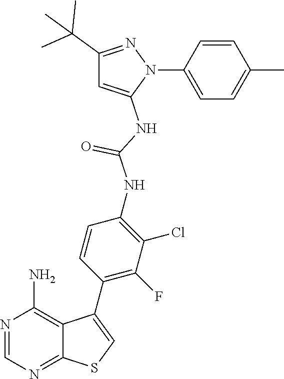 [SMILES: Cc1ccc(-n2nc(C(C)(C)C)cc2NC(=O)Nc2ccc(-c3csc4ncnc(N)c34)c(F)c2Cl)cc1] |
| If-24 | 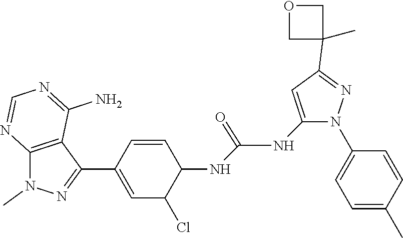 |
| If-25 | 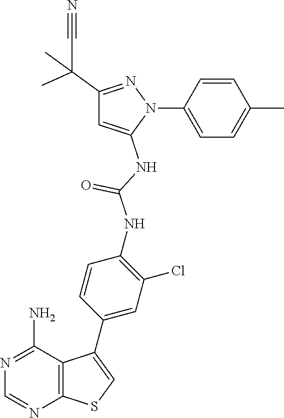 [SMILES: Cc1ccc(-n2nc(C(C)(C)C#N)cc2NC(=O)Nc2ccc(-c3csc4ncnc(N)c34)cc2Cl)cc1] |
| If-26 | 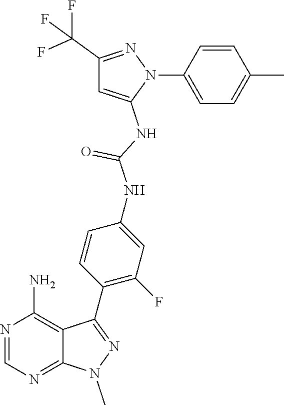 [SMILES: Cc1ccc(-n2nc(C(F)(F)F)cc2NC(=O)Nc2ccc(-c3nn(C)c4ncnc(N)c34)c(F)c2)cc1] |
| If-27 | 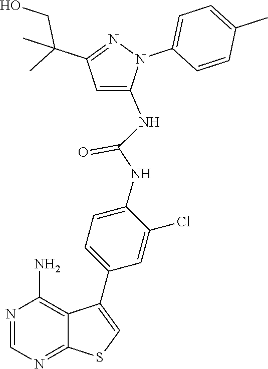 [SMILES: Cc1ccc(-n2nc(C(C)(C)CO)cc2NC(=O)Nc2ccc(-c3csc4ncnc(N)c34)cc2Cl)cc1] |
| If-28 | 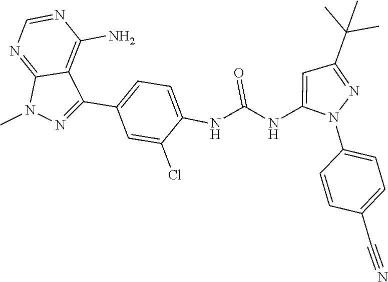 [SMILES: Cn1nc(-c2ccc(NC(=O)Nc3cc(C(C)(C)C)nn3-c3ccc(C#N)cc3)c(Cl)c2)c2c(N)ncnc21] |
| If-29 | 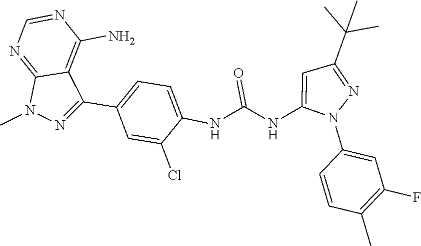 [SMILES: Cc1ccc(-n2nc(C(C)(C)C)cc2NC(=O)Nc2ccc(-c3nn(C)c4ncnc(N)c34)cc2Cl)cc1F] |

It is understood that in the present description, combinations of substituents and/or variables of the depicted formulae are permissible only if such contributions result in stable compounds.

In some embodiments, the composition is in vivo. In certain more specific embodiments, the composition is in vitro.

In certain specific embodiments, NEK7 small molecule inhibitor compound is in contact with at least one NEK7 protein in a type 2 binding mode. In certain embodiments, NEK7 small molecule inhibitor compound is a modulator of the NLRP3 inflammasome. In certain embodiments,

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

The following General Reaction Schemes illustrate exemplary methods for preparation of compounds of Structure (Ia):

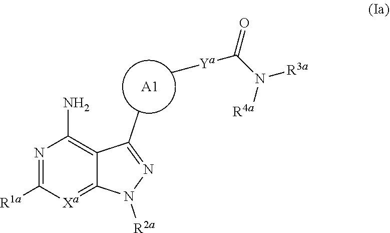

or pharmaceutically acceptable salts, stereoisomers or prodrug thereof, wherein each of A1, X^{a}, Y^{a}, R^{1a}, R^{2a}, R^{3a} and R^{4a} are as defined herein.

### General Reaction Scheme Ia-1

The following General Reaction Scheme, wherein X^{1a} is halogen, and X^{a}, R^{1a}, R^{2a}, and A1 have the meanings described herein, illustrates examples of methods of making the amine Intermediates Ia-B:

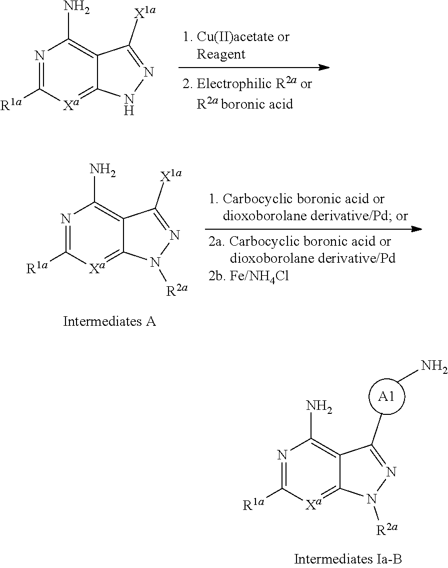

As shown in General Reaction Scheme Ia-1, functionalization of the pyrazolopyrimidine with an alkyl or aryl boronic acid or an appropriate electrophile in presence of a base affords Intermediate Ia-A which can then be subject to palladium catalyzed arylation (followed as needed by a reduction step) to form amine Intermediate Ia-B.

### General Reaction Scheme Ia-2

The following General Reaction Scheme illustrates examples of methods of making the carbamate Intermediates Ia-C:

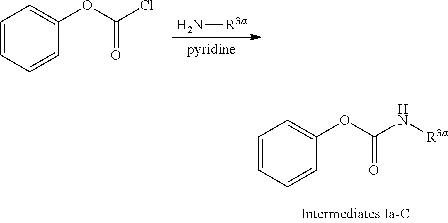

As shown in General Reaction Scheme Ia-2, Intermediates Ia-C can be prepared in the presence of a base by reaction of phenyl chloroformates and the indicated heteroaryl amine (an amine-substituted analogue of R^{3a}). General Reaction Scheme Ia-2 depicts preparation of compounds wherein R^{4a} is H; however, compounds wherein R^{4a} is other than H can be prepared by similar methods by installing R^{4a} after preparation of Intermediate Ia-C or by using an appropriately substituted heteroaryl amine.

### General Reaction Scheme Ia-3

The following General Reaction Scheme illustrates examples of methods of making the compounds of Structure (Ia):

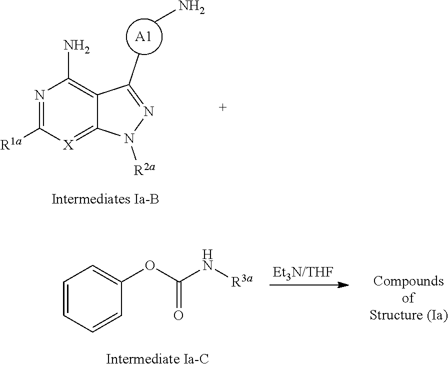

Intermediates Ia-B and Intermediates Ia-C are treated with a base, such as trimethylamine, in THF to afford the compounds of Structure (Ia).

Any of the above reaction schemes can be modified at any step to add and/or modify a substituent or change the order of the steps as appropriate during any stage of the overall synthesis of desired compounds. For example, one of ordinary skill in the art will readily understand that a carbamate analogue of Intermediate Ia-B could alternatively be prepared and reacted with an amine analogue of R^{3a} to prepare compounds of Structure (Ia).

The following General Reaction Schemes illustrate exemplary methods for preparation of compounds of Structure (Ib):

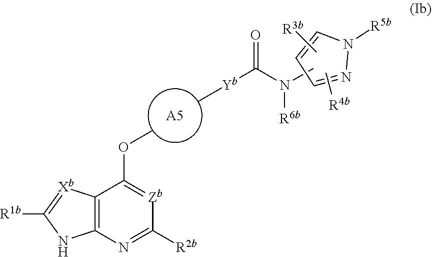

or pharmaceutically acceptable salts, stereoisomers or prodrug thereof, wherein each of A2, X^{b}, Y^{b}, Z^{b}, R^{1b}, R^{2b}, R^{3b}, R^{4b}, R^{5b}, and R^{6b} are as defined herein.

### General Reaction Scheme Ib-1

The following General Reaction Scheme, wherein X^{1b} is halogen, and X^{b}, Z^{b}, R^{1b}, R^{2b}, R^{3b}, R^{4b}, R^{5b}, R^{6b}, and R^{8b} have the meanings described herein, illustrate examples of methods of making the compounds of Structure (Ib) where Y^{b} is NR^{8b}:

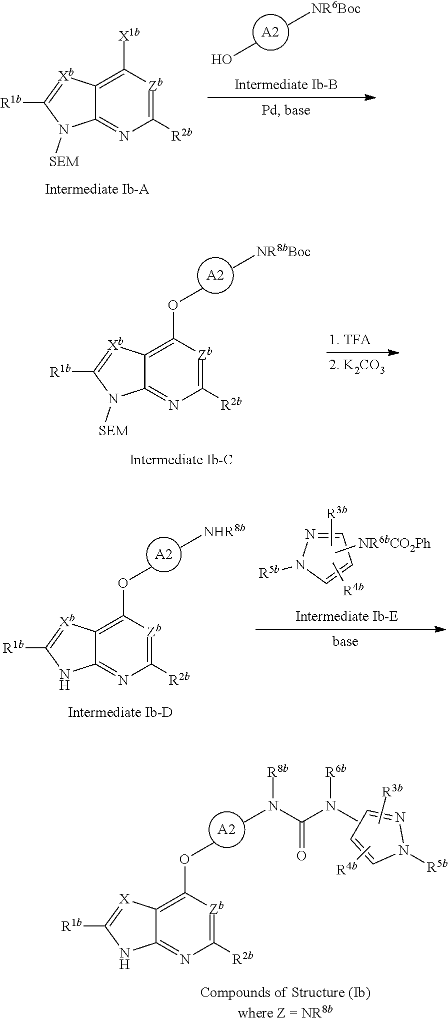

As shown in General Reaction Scheme Ib-1, palladium-catalyzed coupling between the halopyri(mi)dine of the bicyclic system of Intermediate Ib-A and an appropriate alcohol (Intermediate Ib-B) affords aryl ether Intermediate Ib-C. Subsequent de-protection of the amino group with trifluoroacetic acid (which also removes the SEM protecting group to unmask the pyrrole or imidazole NH of the bicyclic core) provides amine Intermediate Ib-D) which is coupled with carbamate Intermediate Ib-E in the presence of a base to afford compounds of Structure (Ib) in which Z^{b} is NR^{8b}.

### General Reaction Scheme Ib-2

The following General Reaction Scheme, wherein X^{1b} is halogen, and X^{b}, Z^{b}, R^{1b}, R^{2b}, R^{3b}, R^{4b}, R^{5b}, R^{6b}, R^{8b}, and R^{9b} have the meanings described herein, illustrate examples of methods of making the compounds of Structure (Ib) where Y^{b} is C(R^{8b})(R^{9b}):

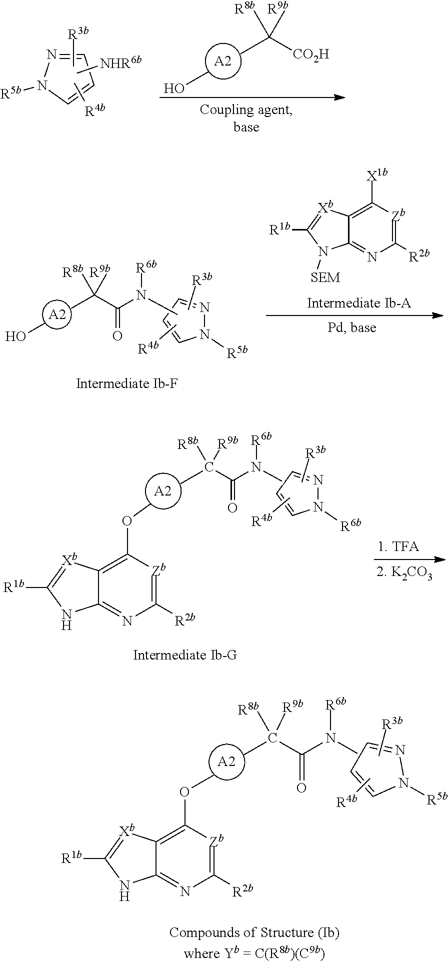

As shown in General Reaction Scheme Ib-2, reaction of the appropriate pyrazole-amine and carboxylic acid in the presence of a coupling agent and a base yields alcohol Intermediate Ib-F which can be coupled with the halopyri(mi)dine of the bicyclic system) of Intermediate Ib-A under palladium-catalyzed conditions to afford aryl ether Intermediate Ib-G. Removal of the SEM protecting group with TFA affords compounds of Structure (Ib) in which Z^{b} is C(R^{8b})(R^{9b}).

The following General Reaction Schemes illustrate examples of the disclosure of compounds of Structure (Ic):

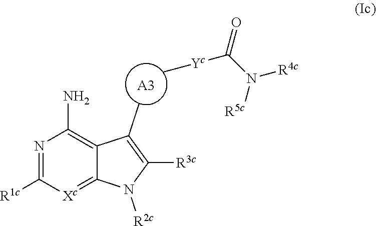

or pharmaceutically acceptable salts, stereoisomers or prodrug thereof, wherein each of A3, X^{c}, Y^{c}, R^{1c}, R^{2c}, R^{3c}, R^{4c} and R^{5c} are as defined herein.

### General Reaction Scheme Ic-1

The following General Reaction Scheme, wherein X^{1c} and X^{2c} are independently halogens, and X^{c}, R^{1c}, R^{2c}, R^{3c} and A3 have the meanings described herein, illustrates examples of methods of making the amine Intermediate Ic-D:

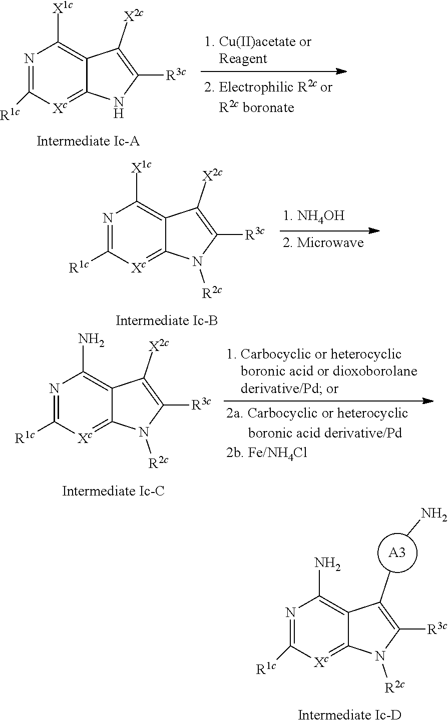

As shown in General Reaction Scheme Ic-1, alkylation of the pyrimidine/pyridine pyrrole (i.e., Intermediate Ic-A) with a cycloalkyl boronate or an appropriate electrophile in presence of base affords the Intermediate Ic-B. This precursor is treated with ammonium hydroxide to form the pyrrolopyrimidine/pyridine-4-amine derivative Intermediate Ic-C. The resulting Intermediate Ic-C can then be subject to palladium catalyzed arylation to form Intermediate Ic-D.

### General Reaction Scheme Ic-2

The following General Reaction Scheme illustrates examples of methods of making the carbamate Intermediate Ic-E:

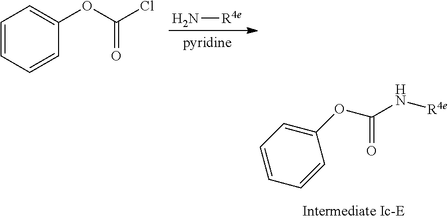

As shown in General Reaction Scheme 2, Intermediate Ic-E can be prepared the in presence of base by reaction of phenyl chloroformates and the indicated heteroaryl amine (an amine-substituted analogue of R^{4c}). General Reaction Scheme Ic-2 depicts preparation of compounds wherein R^{5e} is H; however, compounds wherein R^{5e} is other than H can be prepare by similar methods by instilling R^{5e} after preparation of Intermediate Ic-E, or by using an appropriately substituted heteroaryl amine

### General Reaction Scheme Ic-3

The following General Reaction Scheme illustrates examples of methods of making the compounds of Structure (Ic):

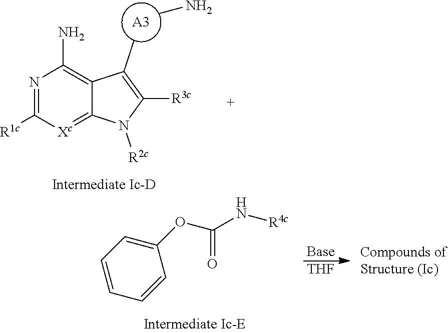

Intermediate Ic-D and Intermediate Ic-E are treated with a base (e.g., triethylamine, DIPEA, DMAP, and the like) in THF to afford the compounds of Structure (Ic).

### General Reaction Scheme Ic-4

The following General Reaction Scheme illustrates examples of methods of making the compounds of Structure (I):

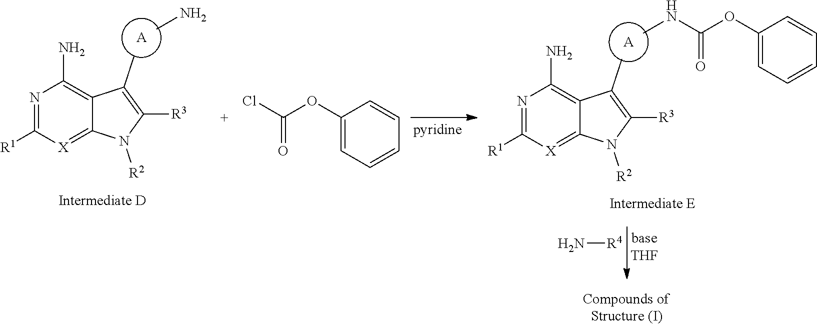

Intermediate D is reacted with the phenyl carbonochloridate shown under appropriate conditions to yield Intermediate E. Intermediate E is then coupled with the amine using a suitable base (e.g., trimethylamine, DIPEA, DMAP, and the like) in THF to afford the compounds of Structure (I).

The following General Reaction Schemes illustrate exemplary methods for preparation of compounds of Structure (Ie):

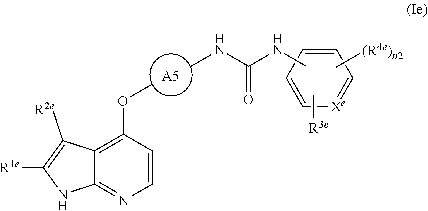

or pharmaceutically acceptable salts, stereoisomers or prodrug thereof, wherein each of X^{e}, A5, R^{1e}, R^{2e}, R^{3e}, R^{4e}, and n2 are as defined herein.

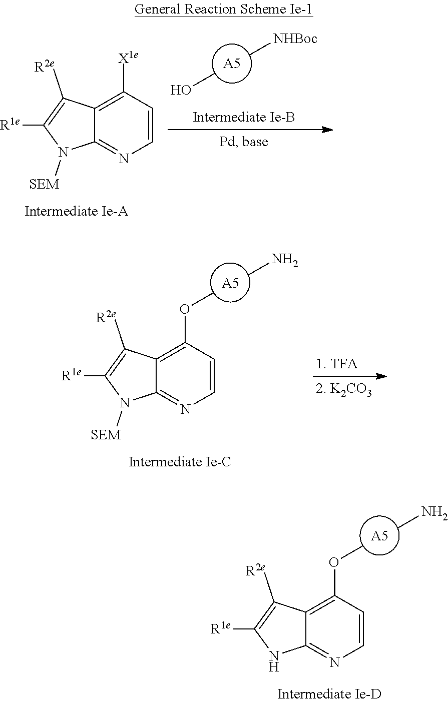

As shown in General Reaction Scheme Ie-1, palladium-catalyzed coupling between the halopyridine (i.e., X^{1e} is a suitable halo substituent) of the bicyclic system of Intermediate Ie-A and an appropriate alcohol (Intermediate Ie-B) affords aryl ether Intermediate Ie-C. Subsequent de-protection of the amino group with trifluoroacetic acid (which also removes the SEM protecting group to unmask the NH of the bicyclic core) provides amine Intermediate Ie-D.

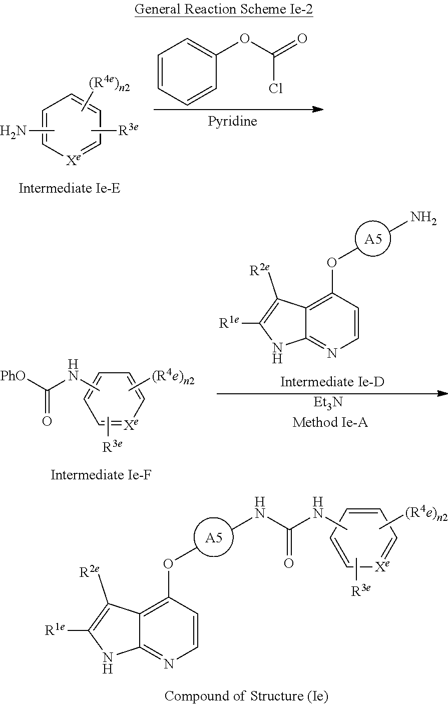

As shown in General Reaction Scheme Ie-2, conversion of amine Intermediate Ie-E to carbamate Intermediate Ie-F in the presence of a base followed by coupling to amine Intermediate Ie-D in the presence of a base affords compounds of Structure (Ie).

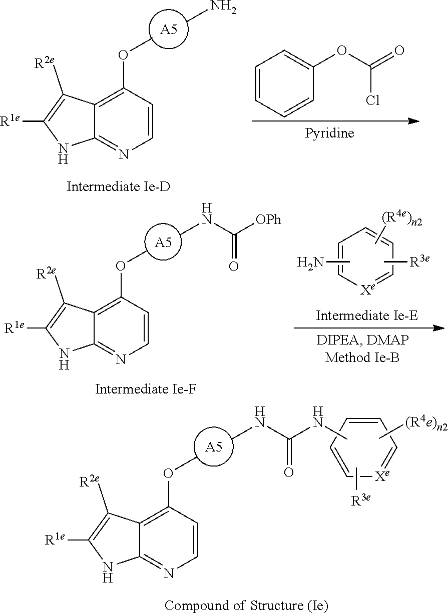

As shown in General Reaction Scheme Ie-3, conversion of amine Intermediate Ie-D to carbamate Intermediate Ie-F in the presence of a base followed by coupling to amine Intermediate Ie-E in the presence of a base affords compounds of Structure (Ie)

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. Prodrugs of compounds of this disclosure are included within the scope of embodiments of the disclosure.

### Pharmaceutical Compositions

Other embodiments are directed to pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection in still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Structure (I), (Ia), (Ib), (Ic), (Id), (Ie), or (If) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses in some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to inter-subject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically Proper formulation is dependent upon the route of administration chosen. Any pharmaceutically acceptable techniques, carriers, and excipients are used as suitable to formulate the pharmaceutical compositions described herein. Remington. The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I), (Ia), (Ib), (Ic), (Id), (Ie), or (If), and a pharmaceutically acceptable carrier.

Provided herein are pharmaceutical compositions comprising one or more compounds selected from compounds of Structure (I), (Ia), (Ib), (Ic), (Id), (Ie), or (If) and pharmaceutically acceptable diluent(s), excipient(s), and carrier(s). In certain embodiments, the compounds described are administered as pharmaceutical compositions in which one or more compounds selected from compounds of Structure (I), (Ia), (Ib), (Ic), (Id), (Ie), or (If) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I), (Ia), (Ib), (Ic), (Id), (Ie), or (If)

In a certain embodiment, pharmaceutical compositions of the compounds of Structure (I), (Ia), (Ib), (Ic), (Id), (Ie), or (If) are modulators of the NLRP3 inflammasome.

In a specific embodiment, pharmaceutical compositions of the compounds of Structure (I), (Ia), (Ib), (Ic), (Id), (Ie), or (If) inhibit NEK7 when administered to a patient or a biological sample.

A pharmaceutical composition, as used herein, refers to a mixture of one or more compounds selected from compounds of Structure (I), (Ia), (Ib), (Ic), (Id), (Ie), or (If) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I), (Ia), (Ib), (Ic), (Id), (Ie), or (If) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I), (Ia), (Ib), (Ic), (Id), (Ie), or (If) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I), (Ia), (Ib), (Ic), (Id), (Ie), or (If) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I), (Ia), (Ib), (Ic), (Id), (Ie), or (If) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Structure (I), (Ia), (b), (Ic), (Id), (Ie), or (If), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I), (Ia), (Ib), (Ic), (Id), (Ie), or (If) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I), (Ia), (Ib), (Ic), (Id), (Ie), or (If). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (l) pentosan polysulfate and other heparinoids, (m) divalent cations such as magnesium and zinc, or (n) combinations thereof.

In some embodiments, the concentration of one or more compounds selected from compounds of Structure (I), (Ia), (Ib), (Ic), (Id), (Ie), or (If) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25%, 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I), (Ia), (Ib), (Ic), (Id), (Ie), or (If) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I), (Ia), (Ib), (Ic), (Id), (Ie), or (If) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I), (Ia), (Ib), (Ic), (Id), (Ie), or (If) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods

Embodiments of the present disclosure are useful as modulators of the NLRP3 inflammasome via the inhibition of NEK7 in a host species. Therefore, the compounds of Structure (I), (Ia), (Ib), (Ic), (Id), (Ie), or (If) are also useful in the treatment of conditions mediated by effector signaling molecules like IL-1β and IL-18.

The host or patient can belong to any mammalian species, for example a primate species, particularly humans, rodents, including mice, rats and hamsters; rabbits, horses, cows, dogs, cats, etc. Animal models are of interest for experimental investigations, providing a model for treatment of human disease.

In one embodiment, the present disclosure is useful as an inhibitor of the NLRP3 inflammasome activation mechanism. Therefore, the compounds of Structure (I), (Ia), (Ib), (Ic), (Id), (Ie), or (If) are also useful in the treatment of conditions resulting from that activation in a host species.

In another embodiment, the compounds of Structure (I), (Ia), (Ib), (Ic), (Id), (Ie), or (If) are useful as inhibitors of the NLRP3 (protein)-NEK7 (protein) interaction. Therefore, the compounds are also useful in the treatment of conditions resulting from the association of NLRP3-NEK7 in a host species.

In certain embodiments, the compounds of Structure (I), (Ia), (Ib), (Ic), (Id), (Ie), or (If) are useful in treating human conditions mediated by effectors selected from the group consisting of IL-1β, IL-18, and caspase-1.

Embodiments of the disclosure also relate to the use of compounds according to Structure (I), (Ia), (Ib), (Ic), (Id), (Ie), or (If) and/or physiologically acceptable salts thereof for the prophylactic or therapeutic treatment and/or monitoring of diseases that are caused, mediated and/or modulated by the NLRP3 inflammasome activity. Furthermore, embodiments of the disclosure relate to the use of compounds according to Structure (I), (Ia), (Ib), (Ic), (Id), (Ie), or (If) and/or physiologically acceptable salts thereof for the production of a medicament for the prophylactic or therapeutic treatment and/or monitoring of diseases that are caused, mediated and/or modulated by NLRP3 inflammasome activity. In certain embodiments, the disclosure provides the use of a compound according to Structure (I), (Ia), (Ib), (Ic), (Id), (Ie), or (If) or physiologically acceptable salts thereof, for the production of a medicament for the prophylactic or therapeutic treatment of a NLRP3-mediated disorder.

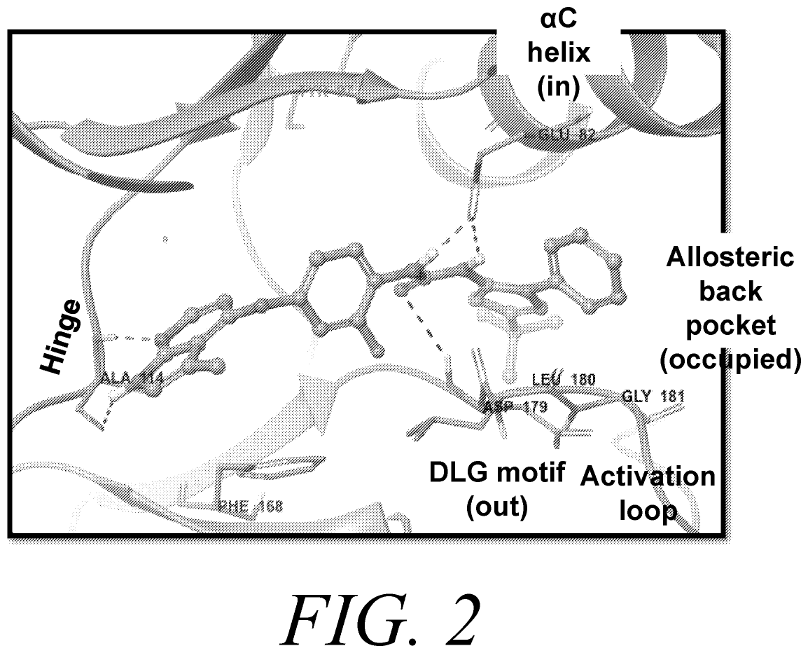

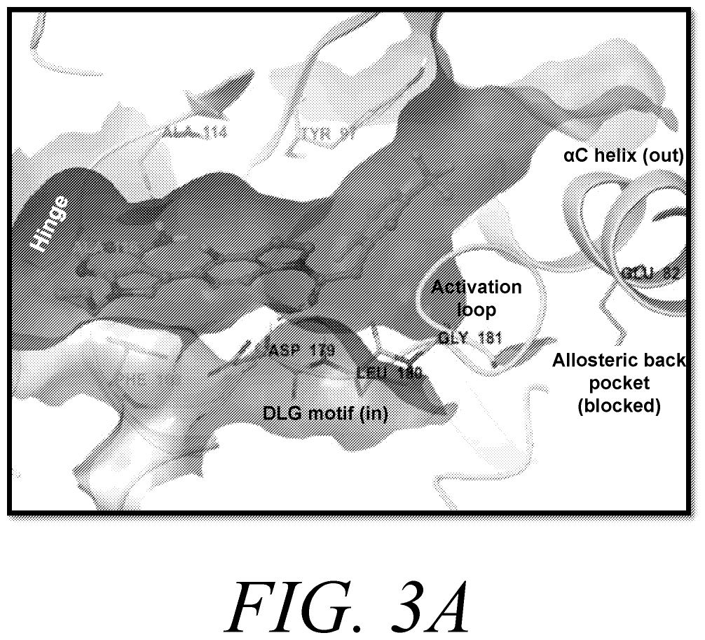

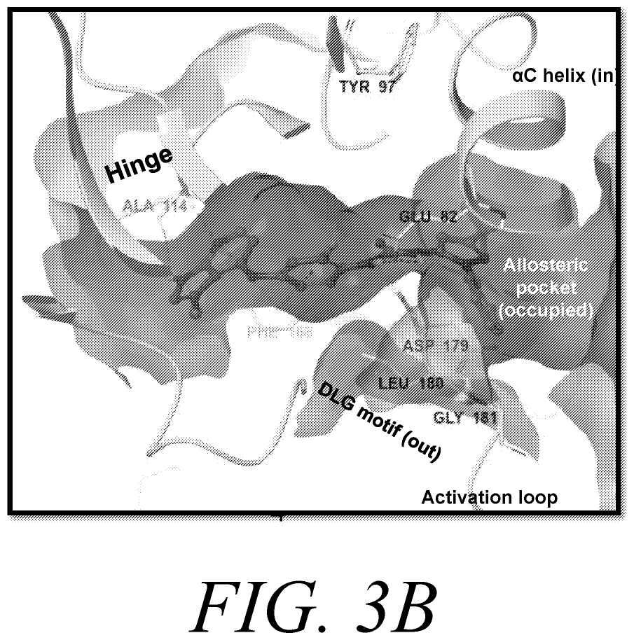

Without wishing to be bound by theory, inventors of the present disclosure have discovered that compounds of the present disclosure bind via a specific binding mode in which the hinge-binding element interacts with one or two hinge residues of NEK7 (ALA114 in all cases, ALA114 and ALA112 in others) via hydrogen bonding. Additionally, in some embodiments, two NH groups of the urea-type linker interact in a bidentate fashion via direct hydrogen bonds with GLU82 (see, e.g., FIG. 2). This interaction helps to stabilize the αC helix in an inward position. NEK7 small molecule inhibitors of this type assist in the formation of a network of direct or water-mediated hydrogen bonding between the compounds and ASP179 of NEK7. The hydrogen bonding stabilizes the DLG loop in the “out” position (i.e., distal from the hinge-binding element) allowing access to the allosteric back pocket which is occupied by the hydrophobic back pocket group of the NEK7 small molecule inhibitor. These features are typical of a type-2 binding mode in which the catalytically inactive conformation of NEK7 is stabilized upon binding the small molecule inhibitor. This conformation is expected to display increased rigidity as a consequence of an extensive network of hydrogen bonds connecting the small molecule inhibitor with various structural features of NEK7.

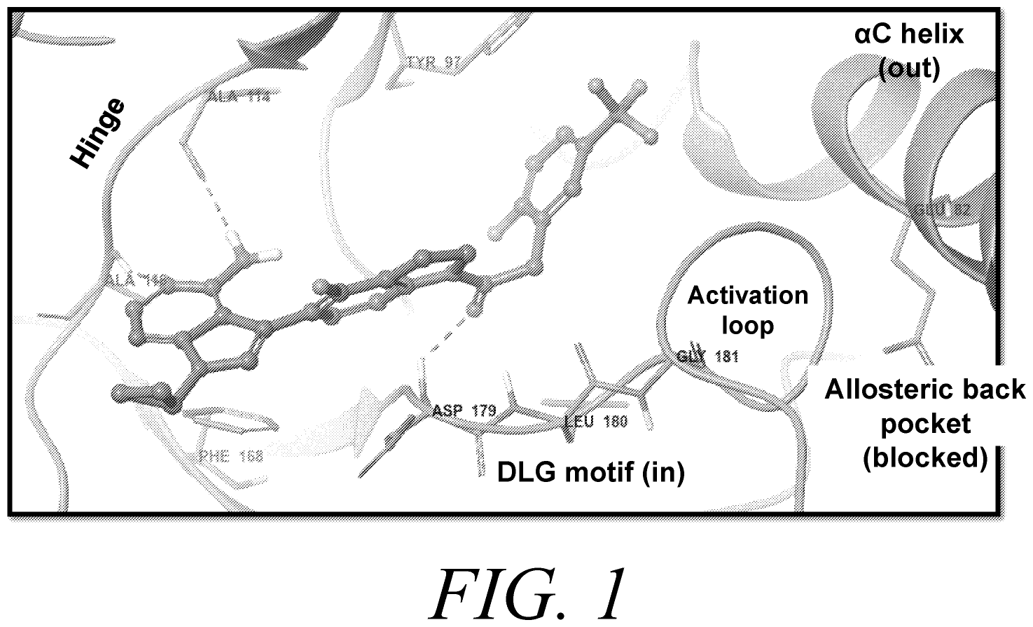

Conversely, compounds having similar structural features, such as compound of Structure (II), likely bind via a different binding mode (see, e.g., Biological Example 4 and FIG. 1). A small molecule compound that exhibits a type 1.5 binding mode interacts with (i) ALA116 and ALA114 via a pair of hydrogen bonds (dashed lines); (ii) ASP179 of the DLG loop (dashed lines) stabilizing the loop in the “in” position and restricting access to the allosteric back pocket. Accordingly, no interactions are observed between such a small molecule compound and the αC helix that is positioned outward. This type-1.5 binding also results in the stabilization of an inactive conformation of NEK7, albeit one that is expected to display lesser rigidity compared to the type-2 conformation described above, a consequence of a less extensive network of hydrogen bonds characterized by a conformation which features a weaker network of hydrogen bonds connecting the small molecule inhibitor with various structural features of NEK7.

e.g.i.e.e.g.e.g. Accordingly, one embodiment provides a method of treating or preventing a disease or disorder, the method comprising administering a NEK7 small molecule inhibitor compound to a subject in need thereof, the NEK7 small molecule inhibitor compound comprising at least one of the following features:
i. a hinge-binding element comprising at least one hydrogen donor and at least one hydrogen acceptor;
ii. a flexible linker;
iii. a urea-type linker, or
iv. a hydrophobic back pocket group.

In some embodiments, hinge-binding element comprises a hydrogen attached to a nitrogen as a hydrogen donor. In certain embodiments, the hinge-binding element comprises a nitrogen with a lone electron pair as a hydrogen acceptor. In some more specific embodiments, the hinge-binding element comprises a heteroaryl. In more specific embodiments, the hinge-binding element comprises a bicyclic heteroaryl. In more specific embodiments, the hinge-binding element comprises a fused bicyclic heteroaryl in certain embodiments, the hinge-binding element has the following structure:

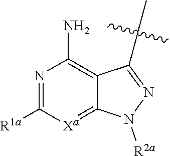

wherein:

X^{a} is N or CH;

R^{1a} is H or C₁-C₆ alkyl; and

R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl.

In more specific embodiments, X^{a} is N. In some embodiments. R^{1a} is H or methyl. In certain embodiments, R^{2a} has one of the following structures:

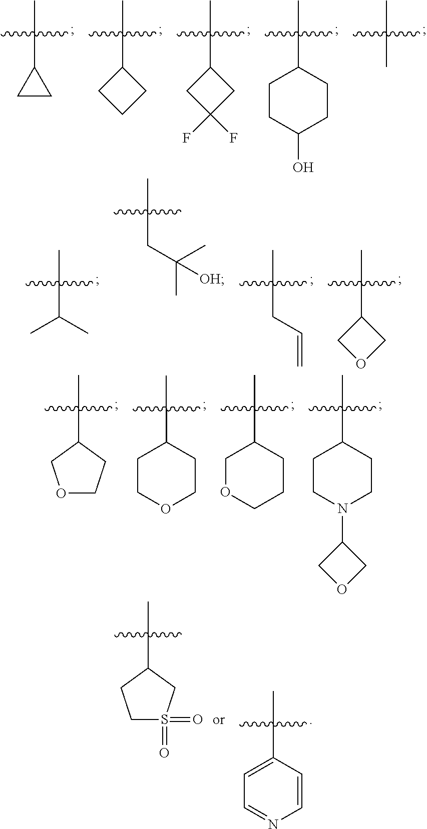

In certain embodiments, the hinge-binding element has the following structure:

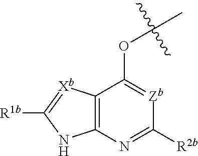

wherein:

X^{b} is N or CR^{10b};

Z^{b} is N or CR^{11b}; and

R^{1b}, R^{2b}, R^{10b}, and R^{11b} are each independently H, halo, C₁-C₆ alkyl, or C₃-C₈ cycloalkyl.

In certain embodiments, X^{b} is N or CH. In some embodiments, X^{b} is CR^{10b} and R^{11b} is chloro, methyl, or cyclopropyl. In certain embodiments, Z^{b} is N or CH. In more specific embodiments, Z^{b} is CR^{11b} and R^{11b} is halo, C₁-C₆ alkyl, or C₃-C₈ cycloalkyl. In some embodiments, R^{1b} is H or methyl. In some embodiments, R^{2b} is H. In some embodiments, the hinge-binding element has the following structure:

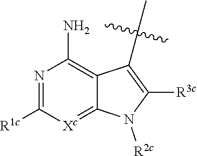

wherein:

X^{c} is CH or N;

R^{1c} is H or C₁-C₆ alkyl:

R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl; and

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl and C₁-C₆ alkoxy.

In some embodiments, X^{c} is N. In certain embodiments, X^{c} is CH In some more specific embodiments, R^{1c} is H or methyl.

In some embodiments, R^{2c} has one of the following structures:

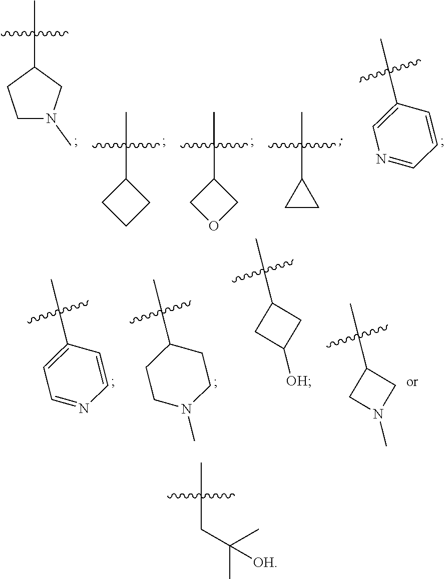

In certain embodiments, R^{3c} is H or methyl. In some embodiments, the hinge-binding element has the following structure:

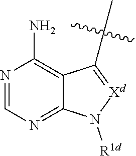

wherein:

X^{d} is N or CR^{4d};

R^{1d} is C₁-C₆ alkyl, C₁-C₆ hydroxylalkyl, C₃-C₈ cycloalkyl, or 3-10 membered heterocyclyl; and

R^{4d} is H, C₁-C₆ alkyl, C₁-C₆ haloalkyl, or C₃-C₈ cycloalkyl.

In some embodiments, X^{d} is N In more specific embodiments, X^{d} is CH. In some embodiments, R^{1d} has one of the following structures:

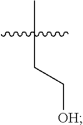 [SMILES: *CCO]

### —CH₃;

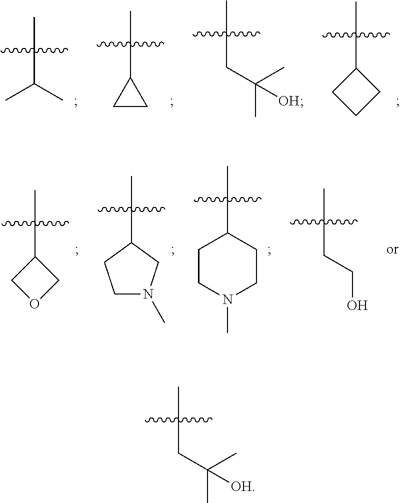

In some embodiments, the hinge-binding element has the following structure:

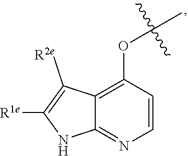

wherein:

R^{1e} is H, halo, C₁-C₆ alkyl, C₃-C₁₀ cycloalkyl, or 3-10 membered heterocyclyl; and

R^{2e} is H, halo, C₁-C₆ alkyl, C₁-C₆ haloalkyl, C₃-C₁₀ cycloalkyl, or 3-10 membered heterocyclyl.

In certain embodiments, R^{1e} is H, methyl, fluoro, or chloro. In more specific embodiments, R^{2e} is chloro, fluoro, cyclopropyl, or methyl.

In some embodiments, the flexible linker comprises at least one cycloalkyl, heterocyclyl, aryl, or heteroaryl. In some embodiments, the flexible linker is monocyclic or bicyclic. In more embodiments, the flexible linker is a fused bicyclic. In certain embodiments, the flexible linker has one of the following structures:

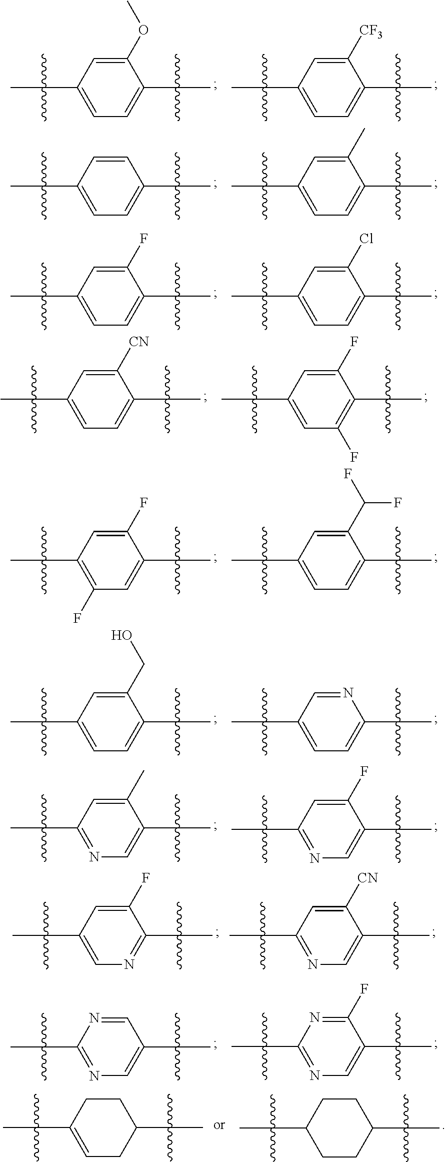

In some embodiments, the urea-type linker comprises the following structure:

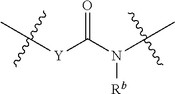

wherein:

Y is C(R^{c})(R^{d}), or NR^{b};

R^{a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl;

R^{b} is H, C₁-C₆ alkyl, C₂-C₆ alkenyl, C₂-C₆ alkynyl, C₃-C₈ cycloalkyl, or C₁-C₆ hydroxylalkyl;

R^{c} is H, C₁-C₆ alkyl, C₂-C₆ alkenyl, C₂-C₆ alkynyl, or C₁-C₆ hydroxylalkyl; and

R^{d} is H, OH, C₁-C₆ alkyl, C₂-C₆ alkenyl, C₂-C₆ alkynyl, or C₁-C₆ hydroxylalkyl.

In some embodiments, the urea-type linker has one of the following structures:

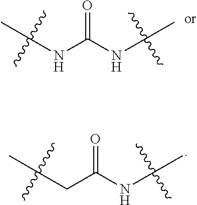 [SMILES: *NC(=O)N* ; *CC(=O)N*]

In some embodiments, the hydrophobic back pocket group comprises an aryl or heteroaryl. In certain embodiments, the hydrophobic back pocket group has one of the following structures:

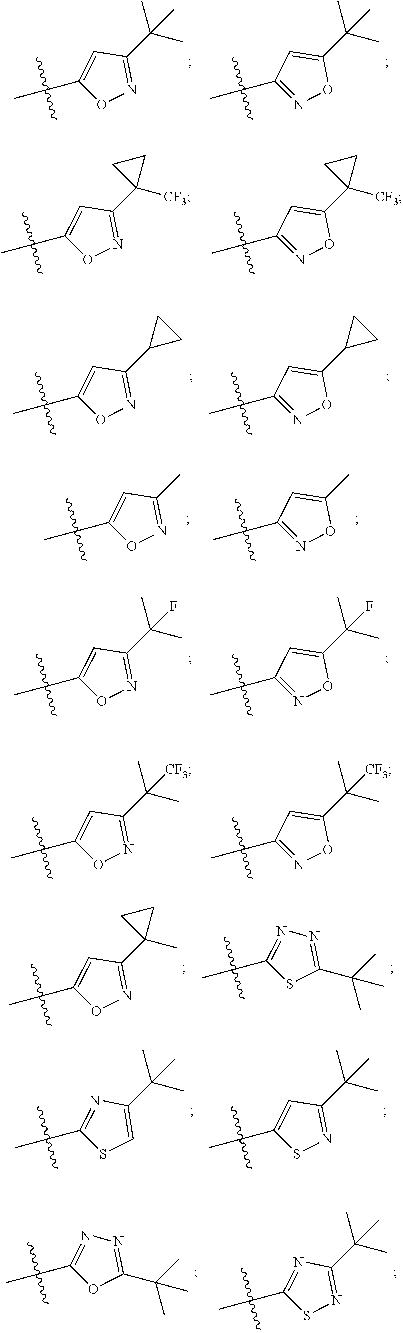

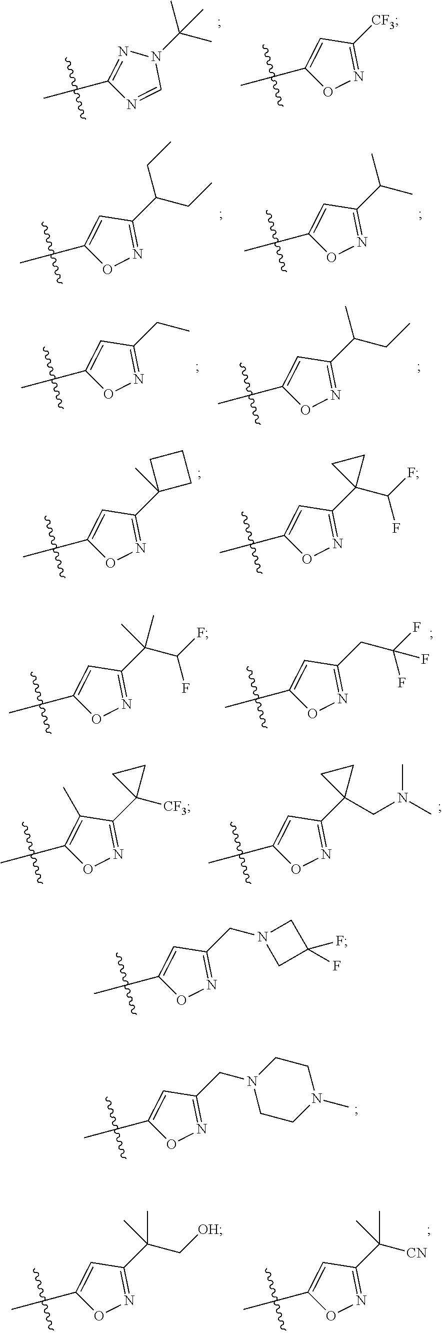

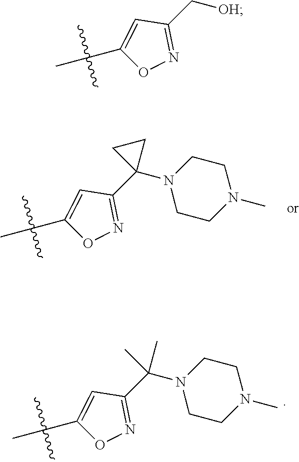 [SMILES: *c1cc(CO)no1 ; *c1cc(C2(N3CCN(C)CC3)CC2)no1 ; *c1cc(C(C)(C)N2CCN(C)CC2)no1]

In some embodiments, hydrophobic back pocket group has the following structure:

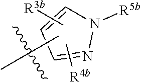

wherein:

R^{3b} and R^{4b} are each independently H, halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl, provided that R^{2a} and R^{2b} are not both H, and

R^{5b} is C₁-C₆ alkyl, C₂-C₆ alkenyl, C₂-C₆ alkynyl, C₁-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy.

In certain embodiments. R^{3b} is tert-butyl, methyl, or cyclopropyl. In certain embodiments, R^{3b} has the following structure:

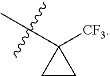 [SMILES: *C1(C(F)(F)F)CC1]

In some embodiments, R^{4b} is H. In certain embodiments, R^{5b} has one of the following structures:
—CH₃;

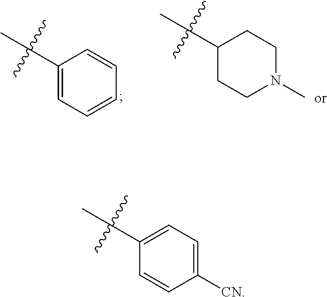

In some embodiments, the hydrophobic back pocket group has one of the following structures:

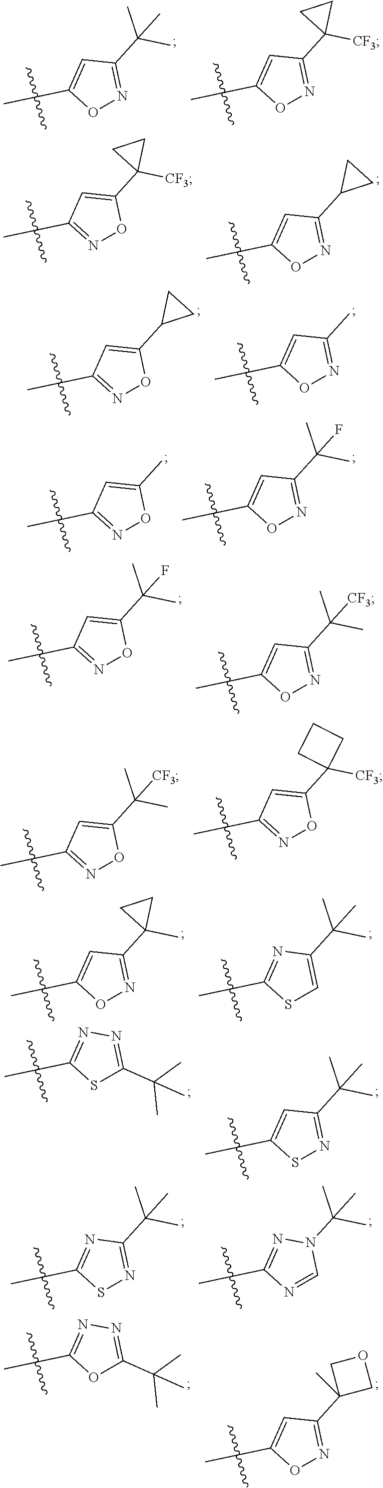

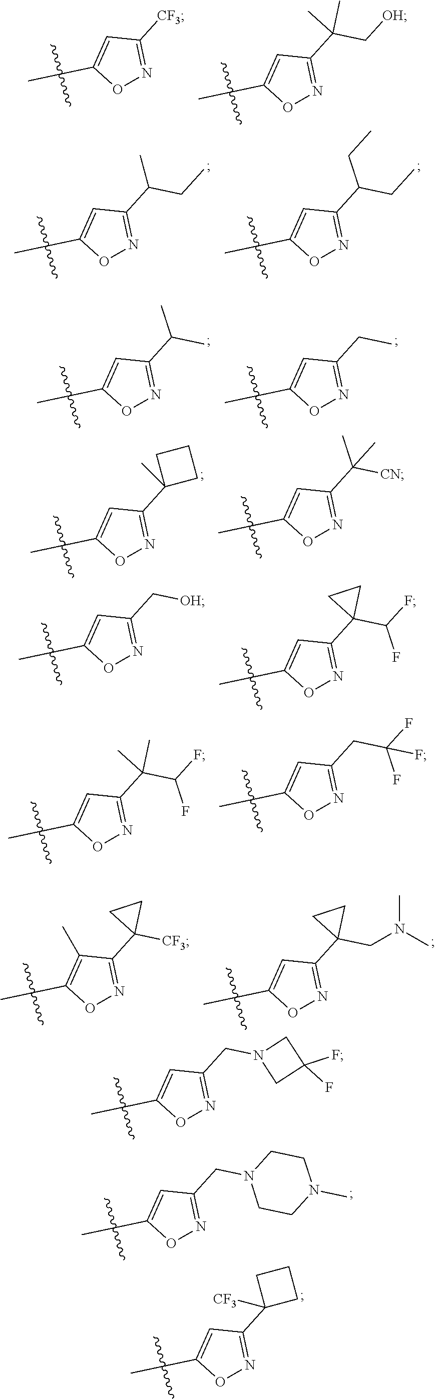

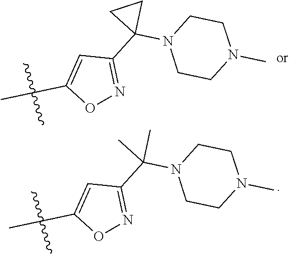

In some embodiments, the hydrophobic back pocket group has the following structure:

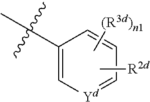

wherein:

Y^{d} is N or CH;

R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;

R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl; and

n1 is 0, 1, 2, 3, or 4.

In some embodiments, Y^{d} is CH. In certain embodiments, Y^{d} is N.

In certain embodiments, R^{2d} has one of the following structures:

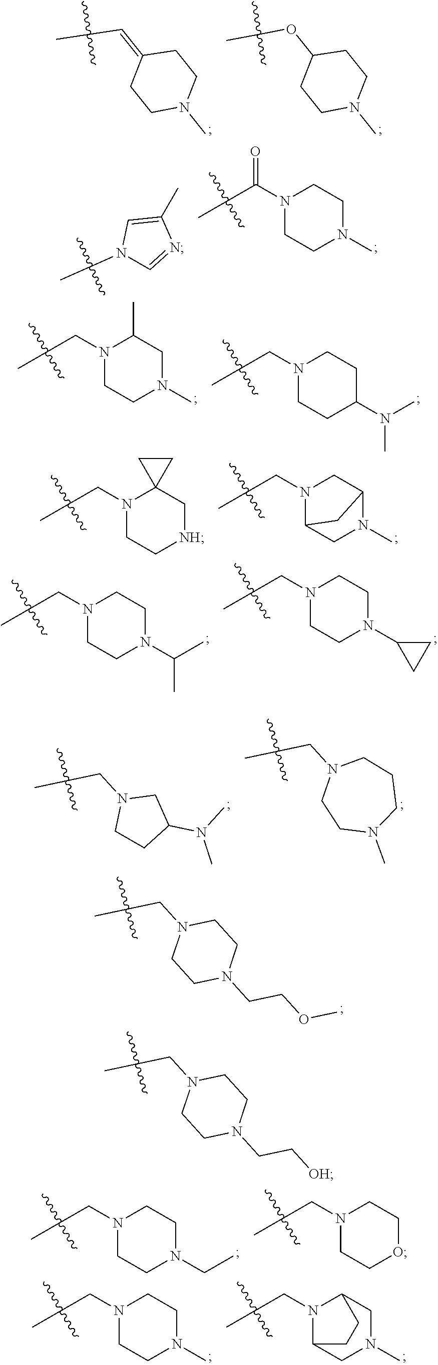

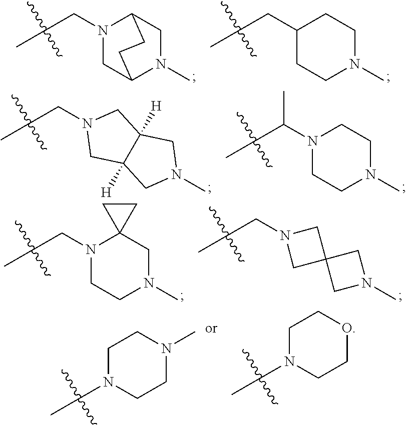

In some specific embodiments, n1 is 0. In certain embodiments, n1 is 1 or 2 and each R^{3d} is independently fluoro, chloro, trifluoromethyl, difluoromethyl, trifluoromethoxy, methyl, methoxy, cyclopropyl, or cyano.

In more specific embodiments, the hydrophobic back pocket group has the following structure:

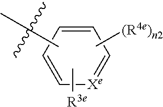

wherein:

X^{e} is N or CH;

R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;

R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and

n2 is 0, 1, 2, 3, or 4.

In some embodiments, X^{e} is CH. In certain embodiments, X^{e} is N. In some embodiments, R^{3e} has one of the following structures:

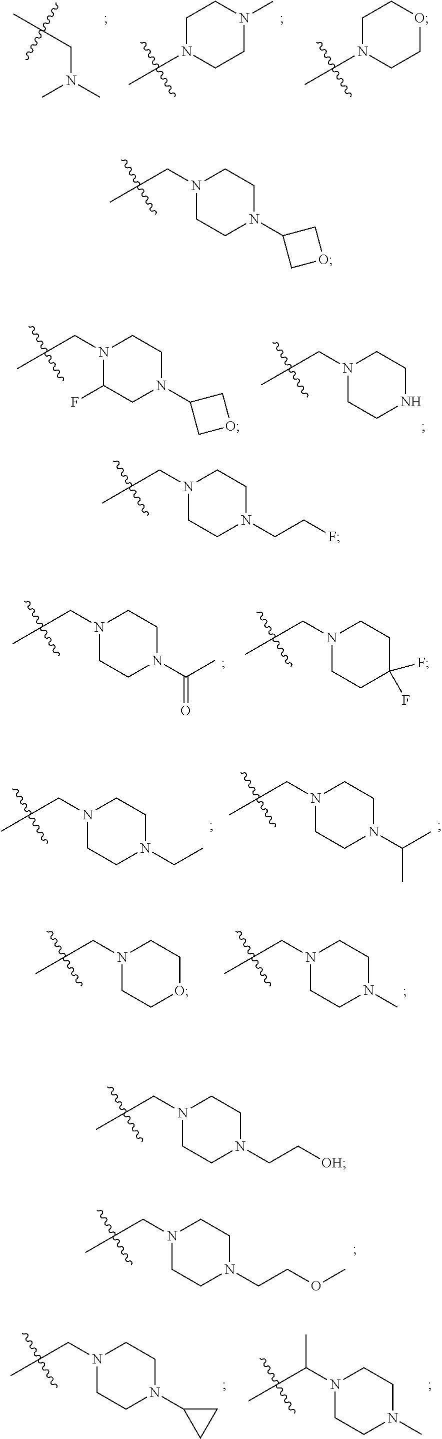

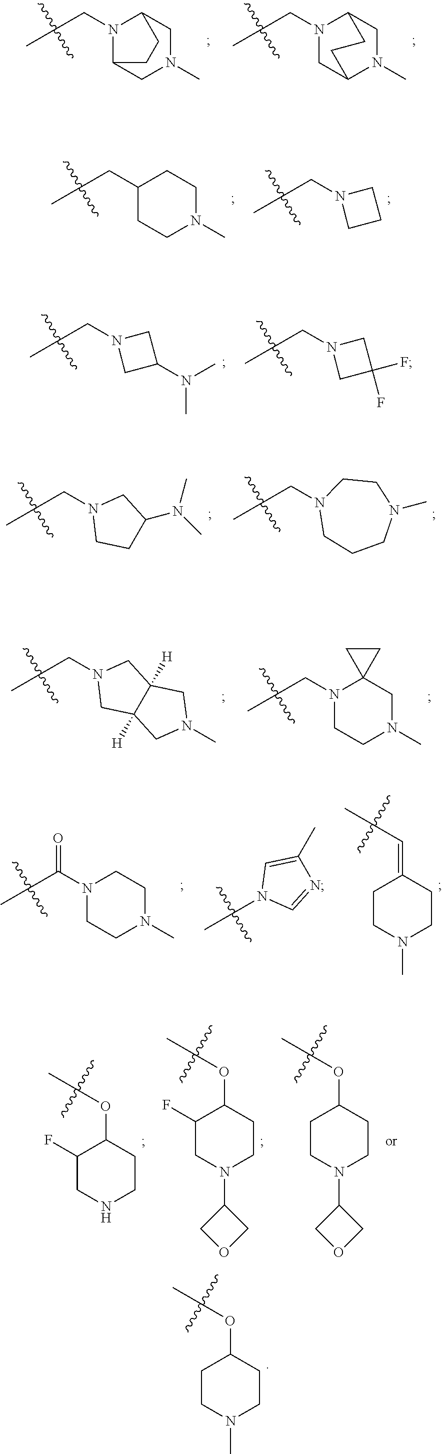

In some embodiments, n2 is 1 and R^{4e} is trifluoromethyl, difluoromethyl, fluoro, chloro, methyl, cyclopropyl, methoxy, or 2,2-difluorocyclopropyl.

In certain embodiments, the NEK7 small molecule inhibitor compound comprises two or more of the following features:
i. a hinge-binding element comprising at least one hydrogen donor and at least one hydrogen acceptor;
ii. a flexible linker;
iii. a urea-type linker; or
iv a hydrophobic back pocket group.

In some embodiments, the NEK7 small molecule inhibitor compound comprises three or more of the following features:
i. a hinge-binding element comprising at least one hydrogen donor and at least one hydrogen acceptor;
ii. a flexible linker,
iii. a urea-type linker; or
iv. a hydrophobic back pocket group.

In more specific embodiments, the NEK7 small molecule inhibitor compound comprises each of the following features:
i. a hinge-binding element comprising at least one hydrogen donor and at least one hydrogen acceptor;
ii. a flexible linker;
iii. a urea-type linker, or
iv. a hydrophobic back pocket group.

In some embodiments, the NEK7 small molecule inhibitor compound is non-peptidic. In some embodiments, the NEK7 small molecule inhibitor compound is synthetic.

In some embodiments, the NEK7 small molecule inhibitor compound has the following Structure (I):

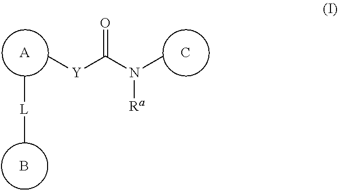

wherein:

A is cycloalkyl, heterocyclyl, aryl, or heteroaryl;

B is a heteroaryl ring;

C is aryl or heteroaryl;

L is a direct bond or —O—:

Y is C(R^{c})(R^{d}), or NR^{b};

R^{a} is H, C₁-C₆, alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl;

R^{b} is H, C₁-C₆ alkyl, C₂-C₆ alkenyl, C₂-C₆ alkynyl, C₃-C₈ cycloalkyl, or C₁-C₆ hydroxylalkyl.

R^{c} is H, C₁-C₆ alkyl, C₂-C₆ alkenyl, C₂-C₆ alkynyl, or C₁-C₆ hydroxylalkyl; and

R^{d} is H, OH, C₁-C₆ alkyl, C₂-C₆ alkenyl, C₂-C₆ alkynyl, or C₁-C₆ hydroxylalkyl.

In some embodiments, A has one of the following structures:

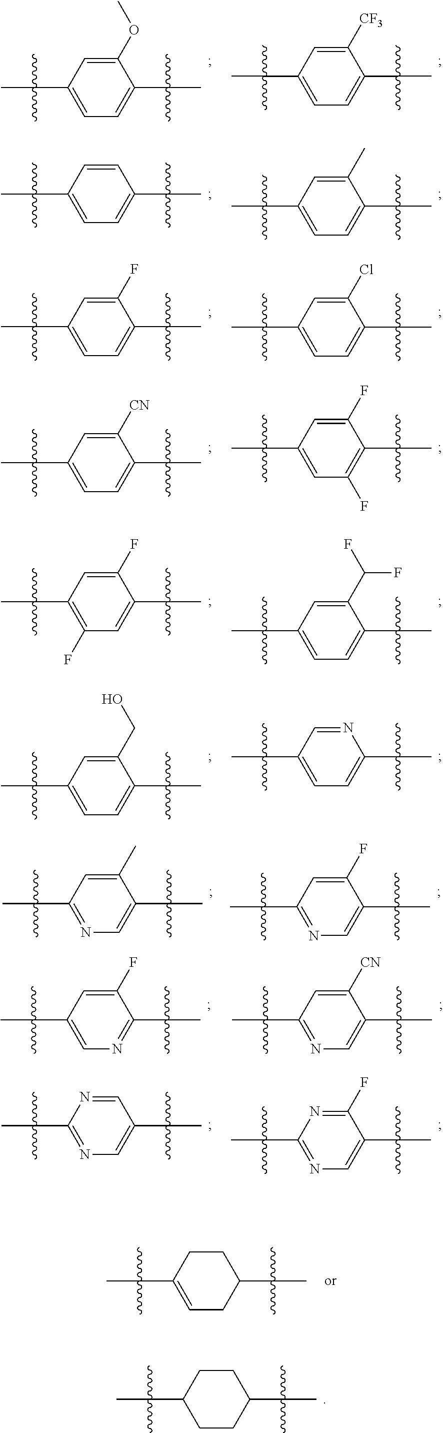

In some embodiments, B is has the following structure:

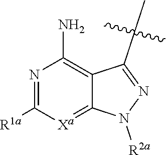

wherein:

X^{a} is N or CH;

R^{1a} is H or C₁-C₆ alkyl; and

R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with JO one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl.

In some embodiments, X^{a} is N. In certain embodiments, R^{1a} is H or methyl. In some embodiments, R^{2a} has one of the following structures:

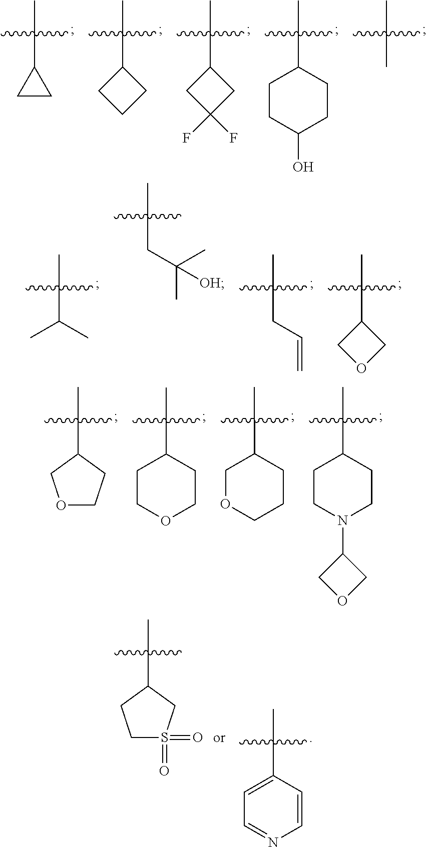

In some embodiments, B has the following structure:

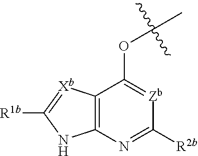

wherein:

X^{b} is N or CR^{10b};

Z^{b} is N or CR^{11b}; and

R^{1b}, R^{2b}, R^{10b}, and R^{11b} are each independently H, halo, C₁-C₆ alkyl, or C₃-C₈ cycloalkyl.x. The method of claim x, wherein X^{b} is N or CH.

In some embodiments, X^{b} is CR^{10b} and R^{11b} is chloro, methyl, or cyclopropyl. In certain embodiments, Z^{b} is N or CH. In certain embodiments, Z^{b} is CR^{11b} and R^{11b} is halo, C₁-C₆ alkyl, or C₃-C₈ cycloalkyl. In more specific embodiments, R^{1b} is H or methyl. In some embodiments, R^{2b} is H.

In certain embodiments, B has the following structure:

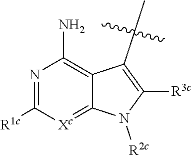

wherein:

X^{c} is CH or N;

R^{1c} is H or C₁-C₆ alkyl;

R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₁-C₆ alkenyl, C₂-C₆ alkynyl, C₁-C₆ alkoxy and 3-8 membered heterocyclyl; and

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₁-C₆ alkynyl and C₁-C₆ alkoxy.

In some embodiments, X^{c} is N. In more specific embodiments, X^{c} is CH. In some embodiments, R^{1c} is H or methyl. In certain embodiments, R^{2c} has one of the following structures:

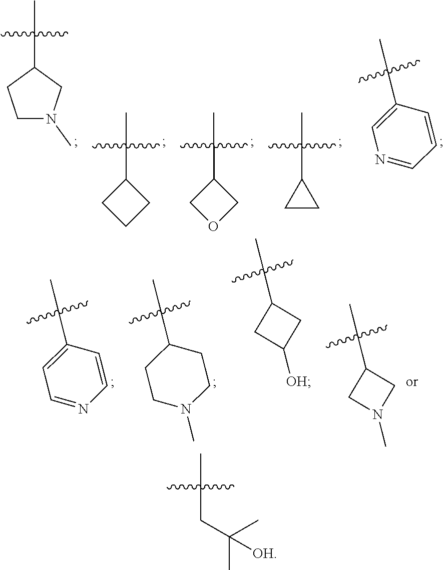

In certain embodiments, R^{3c} is H or methyl. In some embodiments, B has the following structure:

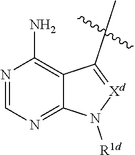

wherein:

X^{d} is N or CR^{4d};

R^{1d} is C₁-C₆ alkyl, C₁-C₆ hydroxylalkyl, C₃-C₁₀ cycloalkyl, or 3-10 membered heterocyclyl; and

R^{4d} is H, C₁-C₆ alkyl, C₁-C₆ haloalkyl, or C₃-C₈ cycloalkyl.

In certain embodiments, X^{d} is N. In some embodiments, X^{d} is CH. In some embodiments, R^{1d} has one of the following structures:

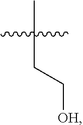 [SMILES: *CCO]

### —CH₃;

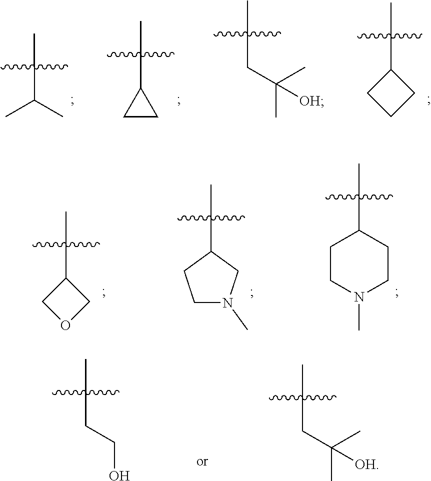

In certain embodiments, B has the following structure:

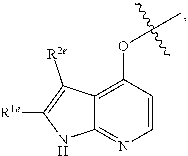

wherein:

R^{1c} is 1, halo, C₁-C₆ alkyl, C₃-C₁₀ cycloalkyl, or 3-10 membered heterocyclyl, and

R^{2c} is H, halo, C₁-C₆ alkyl, C₁-C₆ haloalkyl, C₃-C₁₀ cycloalkyl, or 3-10 membered heterocyclyl.

In some embodiments, R^{1c} is H, methyl, fluoro, or chloro. In some embodiments, R^{2c} is chloro, fluoro, cyclopropyl, or methyl.

In certain embodiments, C has one of the following structures:

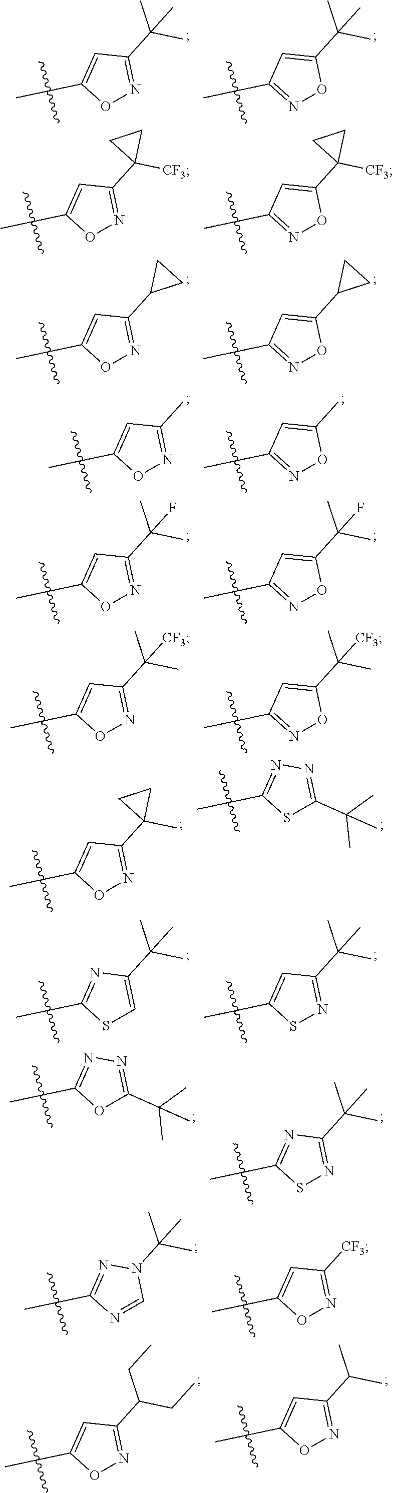

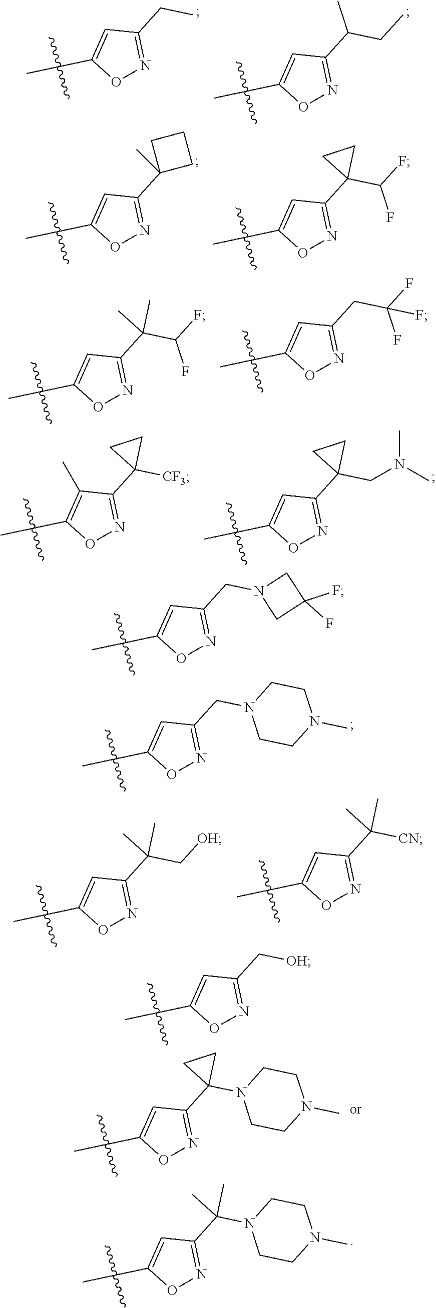

In some embodiments, C has the following structure:

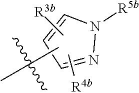

wherein:

R^{3b} and R^{4b} are each independently H, halo, cyano, C₁-C₆ alkyl, C₂-C₆ alkenyl, C₂-C₆ alkenyl, C₁-C₆ haloalkyl, C₁-C₆ alkoxy, C₁-C₆ haloalkoxy, C₃-C₈ cycloalkyl, or C₃-C₈ halocycloalkyl, provided that R^{2a} and R^{2b} are not both H; and

R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy.

In some embodiments, R^{3b} is tert-butyl, methyl, or cyclopropyl. In some more specific embodiments, R^{3b} has the following structure:

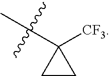 [SMILES: *C1(C(F)(F)F)CC1]

In some embodiments, R^{4b} is H. In more specific embodiments, R^{5b} has one of the following structures:
—CH₃;

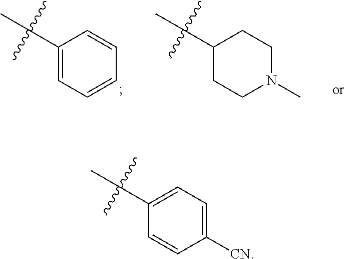

In some embodiments, C has one of the following structures:

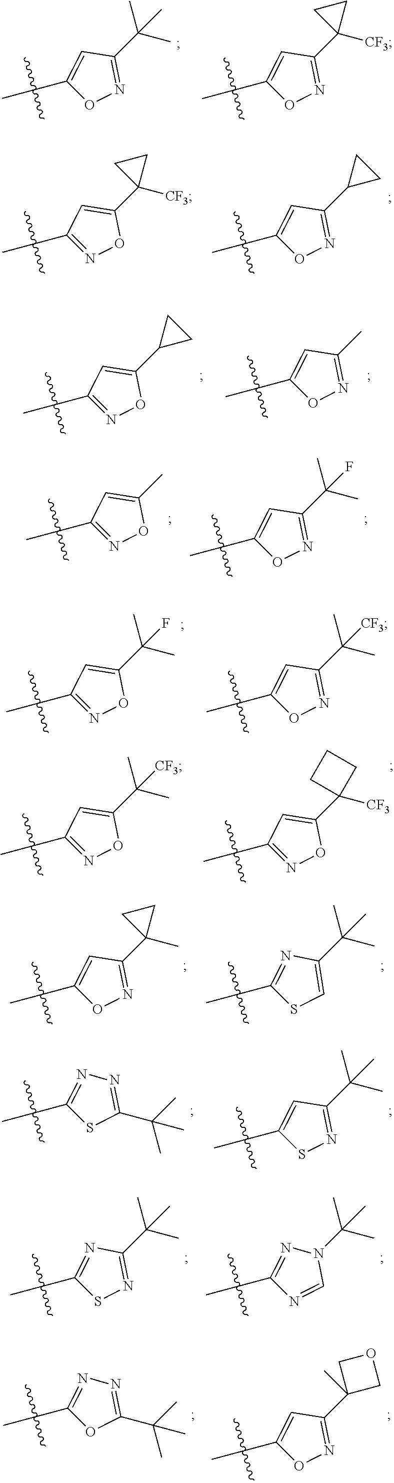

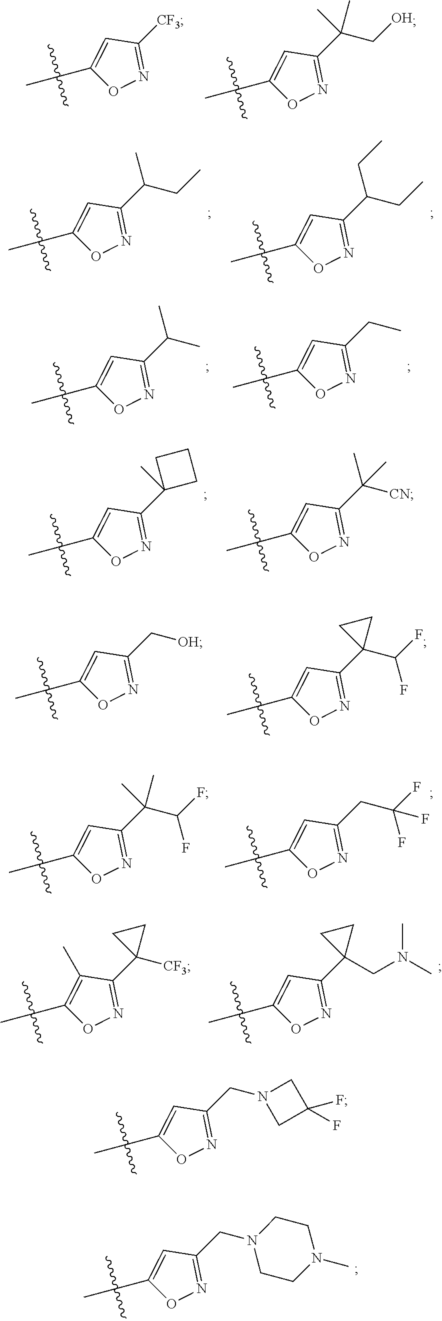

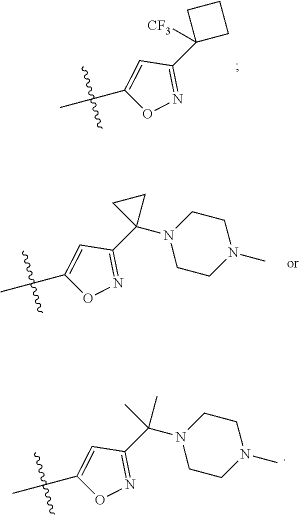 [SMILES: *c1cc(C2(C(F)(F)F)CCC2)no1 ; *c1cc(C2(N3CCN(C)CC3)CC2)no1 ; *c1cc(C(C)(C)N2CCN(C)CC2)no1]

In certain embodiments, C has the following structure:

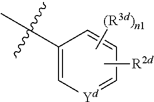

wherein:

Y^{d} is N or CH:

R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;

R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl; and

n1 is 0, 1, 2, 3, or 4.

In some embodiments, Y^{d} is CH. In more specific embodiments, Y^{d} is N.

In some specific embodiments, R^{2d} has one of the following structures:

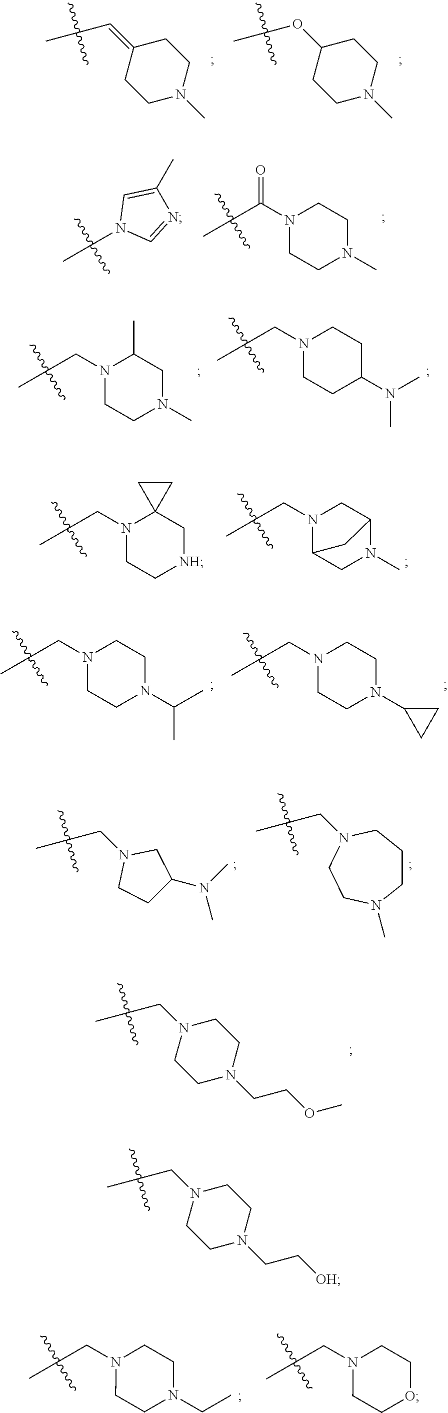

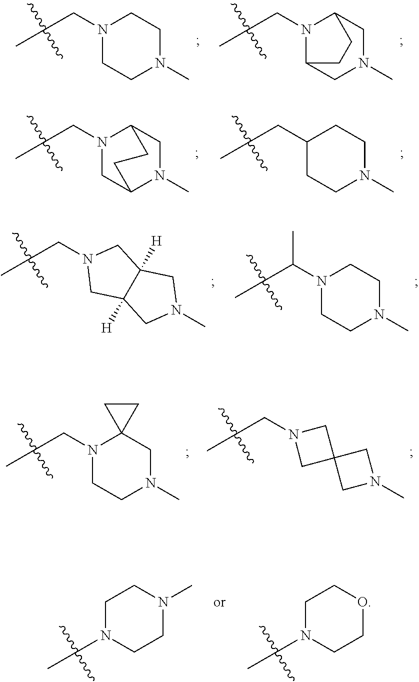

In certain embodiments, n1 is 0. In some embodiments, n1 is 1 or 2 and each R^{3d} is independently fluoro, chloro, trifluoromethyl, difluoromethyl, trifluoromethoxy, methyl, methoxy, cyclopropyl, or cyano. In some embodiments, C has the following structure:

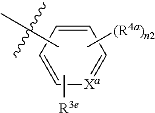

wherein:

X^{e} is N or CH;

R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;

R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and

n2 is 0, 1, 2, 3, or 4.

In certain embodiments, X^{e} is CH. In some embodiments, X^{e} is N. In certain embodiments, R^{3e} has one of the following structures:

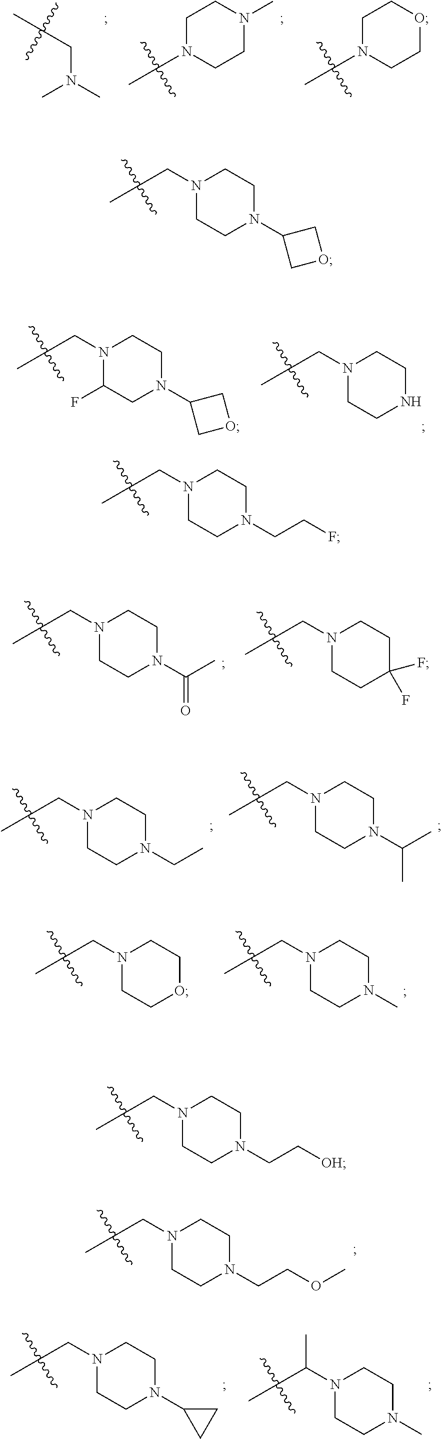

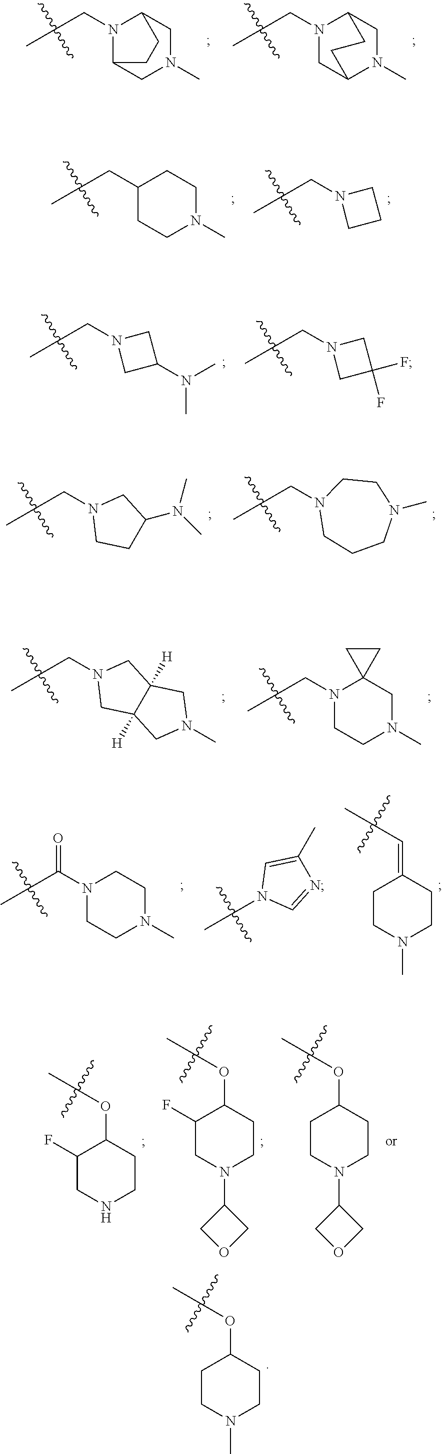

In certain embodiments, n2 is 1 and R^{4e} is trifluoromethyl, difluoromethyl, fluoro, chloro, methyl, cyclopropyl, methoxy, or 2,2-difluorocyclopropyl.

In more specific embodiments, the NEK7 small molecule inhibitor compound comprises two or more of the following features:
i. a hinge-binding element comprising at least one hydrogen donor and at least one hydrogen acceptor;
ii. a flexible linker;
iii. a urea-type linker, or
iv. a hydrophobic back pocket group.

In more specific embodiments, the NEK7 small molecule inhibitor compound comprises three or more of the following features:
i. a hinge-binding element comprising at least one hydrogen donor and at least one hydrogen acceptor;
ii. a flexible linker;
iii. a urea-type linker; or
iv. a hydrophobic back pocket group.

In some embodiments, the NEK7 small molecule inhibitor compound comprises each of the following features
i. a hinge-binding element comprising at least one hydrogen donor and at least one hydrogen acceptor.
ii. a flexible linker;
iii. a urea-type linker; or
iv. a hydrophobic back pocket group.

In certain embodiments, the disorder is a NLRP3-mediated disorder. In more specific embodiments, the disorder is selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions.

In some embodiments, the disorder is selected from type II diabetes, atherosclerosis, Alzheimer's disease, aging, fatty liver, metabolic syndrome, asthma, psoriasis, obesity, acute and chronic tissue damage caused by infection, gout, arthritis, macular degeneration, enteritis, hepatitis, peritonitis, silicosis, UV-induced skin sunburn, contact hypersensitivity, sepsis, cancer, neurodegenerative disease, multiple sclerosis, and Muckle-Wells syndrome. In some embodiments, the disorder is myelodysplastic syndrome (MDS).

In certain specific embodiments, the NEK7 small molecule inhibitor compound has the following Structure (Ia):

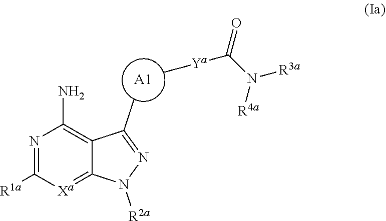

or a pharmaceutically acceptable salt, stereoisomer or prodrug thereof, wherein:

A1 is C₆-C₁₀ aryl, C₃-C₁₀ cycloalkyl, 3-10 membered heterocyclyl or 5-6 membered monocyclic heteroaryl, each of which is optionally substituted with one or more R⁵;

X^{a} is N or CH;

Y^{a} is CHOH or NH;

R^{1a} is H or C₁-C₆ alkyl;

R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;

R^{3a} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from amino, halo, cyano, C₁-C₆ alkyl, C₂-C₆ alkenyl, C₂-C₆ alkynyl, C₁-C₆ haloalkyl, C₃-C₈ cycloalkyl, C₃-C₈ alkylcycloalkyl, C₃-C₈ haloalkylcycloalkyl, C₃-C₈ aminylalkylcycloalkyl, C₁-C₆ cyanoalkyl, C₁-C₆ aminyl, C₁-C₆ hydroxylalkyl, 3-8 membered heterocyclyl, 3-8 membered heterocyclylalkyl, 3-8 membered heterocyclylcycloalkyl, 3-8 membered haloheterocyclyl, 3-8 membered haloheterocyclylalkyl, C₃-C₈ halocycloalkyl and C₃-C₈ halocycloalkylalkyl, and combinations thereof;

R⁴, is H, C₁-C₆ alkyl, C₂-C₆ alkenyl, C₂-C₆ alkynyl, C₃-C₈ cycloalkyl, 3-8 membered heterocyclyl, C₆-C₁₀ aryl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl and C₁-C₆ alkoxy; and

R^{5a} is, at each occurrence, independently halo, cyano, C₁-C₆ alkyl, C₁-C₆ hydroxylalkyl or C₁-C₆ haloalkyl.

In some embodiments, the NEK7 small molecule inhibitor compound has a structure of Table 1a.

In more specific embodiments, the NEK7 small molecule inhibitor compound has the following Structure (Ib):

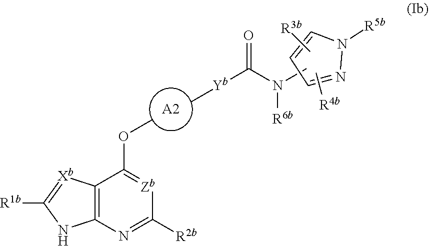

or a pharmaceutically acceptable salt, stereoisomer or prodrug thereof, wherein:

A2 is C₆-C₁₀ aryl, C₃-C₁₀ cycloalkyl, 3-10 membered heterocyclyl, or 5-6 membered monocyclic heteroaryl, each of which is optionally substituted with one or more R^{7b};

X^{b} is N or CR^{10b};

Y^{b} is C(R^{8b})(R^{9b}) or NR^{8b};

Z^{b} is N or CR^{11b};

R^{1b}, R^{2b}, R^{10b}, and R^{11b} are each independently H, halo, C₁-C₆ alkyl, or C₃-C₈ cycloalkyl;

R^{3b} and R^{4b} are each independently H, halo, cyano, C₁-C₆ alkyl, C₂-C₆ alkenyl, C₂-C₆ alkynyl, C₁-C₆ haloalkyl, C₂-C₆ alkoxy, C₁-C₆ haloalkoxy, C₃-C₈ cycloalkyl, or C₃-C₈ halocycloalkyl, provided that R^{2a} and R^{2b} are not both H;

R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy;

R^{6b} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl, each of which is optionally substituted with one more substituents selected from halo, hydroxyl, cyano, aminyl, C₁-C₆ alkyl, C₁-C₆ alkenyl, C₂-C₆ alkynyl, and C₁-C₆ alkoxy;

R^{7b} is, at each occurrence, independently halo, C₁-C₆ alkyl, C₂-C₆ alkenyl, C₂-C₆ alkynyl, or C₁-C₆ haloalkyl;

R^{8b} is H, C₁-C₆ alkyl, C₁-C₆ alkenyl, C₂-C₆ alkynyl, or C₁-C₆ hydroxylalkyl; and

R^{9b} is H, OH, C₁-C₆ alkyl, C₂-C₆ alkenyl, C₂-C₆ alkynyl, or C₁-C₆ hydroxylalkyl.

In certain embodiments, the NEK7 small molecule inhibitor compound has a structure of Table 1b.

In some embodiments, the NEK7 small molecule inhibitor compound has the following Structure (Ic):

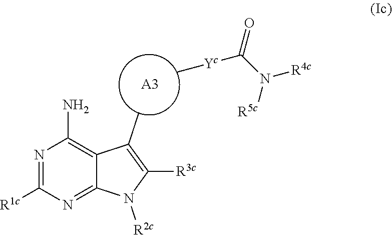

or a pharmaceutically acceptable salt, stereoisomer or prodrug thereof, wherein:

A3 is C₆-C₁₀ aryl, C₁-C₁₀ cycloalkyl, 3-10 membered heterocyclyl or 5-6 membered monocyclic heteroaryl, each of which is optionally substituted with one or more R^{6c};

Y^{c} is CHOH or NH;

R^{1c} is H or C₁-C₆ alkyl;

R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;

R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₁-C₆ alkynyl and C₁-C₆ alkoxy;

R^{4c} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl;

R^{5c} is H, C₁-C₆ alkyl, C₂-C₆ alkenyl, C₂-C₆ alkynyl, C₃-C₈ cycloalkyl, 3- to 8-membered heterocyclyl, C₆-C₁₀ aryl, or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl and C₁-C₆ alkoxy, and

R^{6c} is, at each occurrence, independently halo, C₁-C₆ alkyl, C₁-C₆ alkoxy, cyano, C₁-C₆ hydroxylalkyl or C₁-C₆ haloalkyl.

In some embodiments, the NEK7 small molecule inhibitor compound has a structure of Table 1c.

In some embodiments, the NEK7 small molecule inhibitor compound has the following Structure (Id):

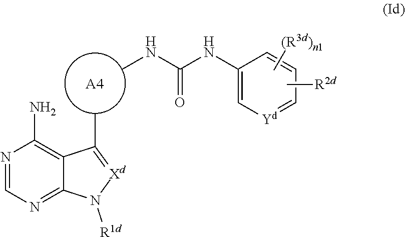

or a pharmaceutically acceptable salt, stereoisomer, or prodrug thereof, wherein:

A4 is C₆-C₁₀ aryl, C₃-C₁₀ cycloalkyl, 3-10 membered heterocyclyl, or 5-6 membered heteroaryl;

X^{d} is N or CR^{4d};

Y^{d} is N or CH:

R^{1d} is C₁-C₆ alkyl, C₁-C₆ hydroxylalkyl, C₃-C₁₀ cycloalkyl, or 3-10 membered heterocyclyl;

R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;

R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl;

R^{4d} is H, C₁-C₆ alkyl, C₁-C₆ haloalkyl, or C₃-C₈ cycloalkyl; and

n1 is 0, 1, 2, 3, or 4.

In certain specific embodiments, the NEK7 small molecule inhibitor has a structure of Table 1d.

S In certain embodiments, the NEK7 small molecule inhibitor compound has the following Structure (Ie):

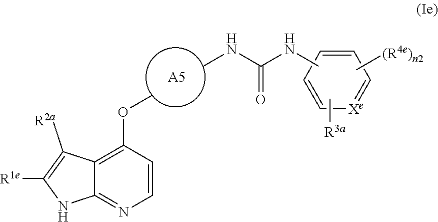

or a pharmaceutically acceptable salt, stereoisomer, or prodrug thereof, wherein:

X^{e} is N or CH, A5 is C₁-C₁₀ arylene, C₃-C₁₀ cycloalkylene, 3-10 membered heterocyclylene, or 5-6 membered heteroarylene;

R^{1e} is H, halo, C₁-C₆ alkyl, C₁-C₁₀ cycloalkyl, or 3-10 membered heterocyclyl;

R^{2e} is H, halo, C₁-C₆ alkyl, C₁-C₆ haloalkyl, C₃-C₁₀ cycloalkyl, or 3-10 membered heterocyclyl;

R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;

R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and

n2 is 0, 1, 2, 3, or 4.

x. The method of claim x, wherein the NEK7 small molecule inhibitor compound has a structure of Table 1e.

In another embodiment, the present disclosure relates to a method of treating inflammatory diseases or conditions mediated by NLRP3 inflammasome by administering to a patient in need thereof a therapeutically effective amount of the compound of Structure (I), (Ia), (Ib), (Ic), (Id), (Ie), or (If).

In some embodiments, the disorders associated with NEK7 which are treatable are selected from rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, type I diabetes, type II diabetes, inflammatory bowel disease (Crohn's Disease and ulcerative colitis), hyperimmunoglobulinemia D and periodic fever syndrome, cryopyrin associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, DIRA (Deficiency of IL-1 Receptor Antagonist), Alzheimer's disease, Parkinson's disease, and Cancer.

Also included herein are methods of treatments in combination with administration of an anti-inflammatory or a therapeutic agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The disclosure also includes embodiments in which the anti-inflammatory agent is a metabolic inhibitor such as a dihydrofolate reductase inhibitor, such as methotrexate or a dihydroorotate dehydrogenase inhibitor, such as leflunomide.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukouienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Other embodiments of the disclosure pertain to combinations in which at least one anti-inflammatory compound is an anti-monoclonal antibody (such as eculizumab or pexelizumab), a TNF antagonist, such as entanercept, or infliximab, which is an anti-TNF alpha monoclonal antibody.

Therapeutic agents used in combination with the compounds of Structure (I), (Ia), (Ib), (Ic), (Id), (Ie), or (If) can also include small molecule compounds that inhibit the activation of NLRP3 inflammasomes, such as MCC950, sulforaphane, isoliquiritigenin, β-hydroxybutyrate, flufenamic acid, mefenamic acid, 3,4-methylenedioxy-p-nitrostyrene (MNS), and parthenolide.

Still other embodiments of the disclosure pertain to combinations in which at least one active agent is an inumunosuppressant compound such as an immunosuppressant compound chosen from methotrexate, leflunomide, cyclosporine, tacrolimus, azathioprine, and mycophenolate mofetil.

The disclosed compounds can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories-Alkylating agents; such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-3024, VAL-0834;

Platinum Compounds such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents, such as amrubicin, bisantrene, decitabine, mitoxantrone, procarbazine, trabectedin, clofarabine; amsacrine, brostallicin, pixantrone, laromustine 1,3;
Topoisomerase Inhibitors: such as etoposide, irinotecan, razoxane, sobuzoxane, teniposide, topotecan; amonafide, belotecan, elliptinium acetate, voreloxin; Microtubule modifiers, such as cabazitaxel, docetaxel, eribulin, ixabepilone, paclitaxel, vinblastine, vincristine, vinorelbine, vindesine, vinflunine; fosbretabulin, tesetaxel; Antirmetabolites: such as asparaginase3, azacitidine, calcium levofolinate, capecitabine, cladribine, cytarabine, enocitabine, floxuridine, fludarabine, fluorouracil, gerncitabine, mercaptopurine, methotrexate, nelarabine, pemetrexed, pralatrexate, azathioprine, thioguanine, carmofur, doxifluridine, elacytarabine, raltitrexed, sapacitabine, tegafur2,3 trimetrexate;
Anticancer antibiotics, such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide 1,3;
Aromatase inhibitors: such as aminoglutethimide, anastrozole, exemestane, fadrozole, letrozole, testolactone; formestane;
Small molecule kinase inhibitors, such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil, anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium, hormones, e.g., cortisone, hydrocortisone or prednisolone: xanthines, e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the compounds of Structure (I), (Ia), (Ib), (Ic), (Id), (Ie), or (If) are administered as a mono-therapy.

For identification of a signal transduction or a mechanistic pathway and for detection of interactions between various signal transduction pathways, various scientists have developed suitable models or model systems, for example cell culture models and models of transgenic animals. For the determination of certain stages in the signal transduction cascade, interacting compounds can be utilized in order to modulate the signal. The compounds of embodiments of the disclosure can also be used as reagents for testing NEK7-dependent signal transduction pathways in animals and/or cell culture models or in the clinical diseases mentioned in this application.

The methods of the disclosure can be performed either in vitro or in vivo. The susceptibility of a particular cell to treatment with the compounds of Structure (I), (Ia), (Ib), (Ic), (Id), (Ie), or (If) can be particularly determined by in-vitro tests, whether in the course of research or clinical application. Typically, a culture of the cell is combined with a compound at various concentrations for a period of time which is sufficient to allow the active agents to inhibit NEK7 activity, usually between about one hour and one week. In-vitro treatment can be carried out using cultivated cells from a biopsy sample or cell line.

In some embodiments, the IC₅₀ of the compounds of Structure (I), (Ia), (Ib), (Ic), (Id), (Ie), or (If) to inhibit NEK7 was determined by the concentration of the compound required to inhibit 50% of the activity of the NEK kinase. The compounds of Structure (I), (Ia), (Ib), (Ic), (Id), (Ie), or (If) exhibited potency values of IC₅₀ of less than about 5 mM, preferably less than about 1 mM and even more preferably less than about 0.100 mM as described in further detail in the Examples.

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

### ExampleS

The following examples are provided for exemplary purposes.

### General Procedures

All proton NMR experiments were recorded on a Bruker NEO Spectrometer equipped with a BBFO probe at 400 MHz. Deuterated solvents contained less than 0.05% v/v tetramethylsilane which was used as the reference signal (set at 0.00 ppm). When deuterated solvents did not contain tetramethylsilane, the residual nondeuterated solvent peaks were used as a reference signal, as per published guidelines (J. Org. Chem. 1997, 62(2H), 7512-7515). Chemical shifts are expressed in parts per million (ppm, 8 units). Coupling constants are in hertz (Hz). Splitting patterns describe apparent multiplicities and are designated as s (singlet), d (doublet), t (triplet), q (quartet), m (multiplet), qt (quintuplet) or brs (broad singlet).

LC/MS analyses were performed on an Agilent Technologies UHPLC 1290 Infinity II with a G6125 MS detector.

Microwave reactions were conducted with a Monowave 300 by Anton Paar GmbH using standard protocols.

### NEK7 Enzymatic Assay

Casein substrate (from bovine milk, hydrolyzed and partially dephosphorylated mixture of α, β and κ caseins, obtained from Sigma Aldrich, catalogue #C4765, diluted in distilled water to a final concentration of 1 mg/mL) and full-length recombinant human NEK7 (expressed by baculovirus in Sf9 insect cells using a N-terminal GST tag, obtained from SignalChem, catalogue #N09-10G, 0.1 μg/μL) were mixed in assay buffer (20 mM Hepes pH 7.5, 10 mM MgCl₂, 1 mM EGTA, 0.02% Brij35, 0.02 mg/ml BSA, 0.1 mM Na₃ VO₄, 2 mM DTT, 1% DMSO). Compounds of interest (serial 3-fold dilution in DMSO from 10 μM to 0.5 nM) or vehicle (1% DMSO) were dispensed into the kinase reaction mixture by Acoustic technology (Echo550, nanoliter range). After incubation at room temperature for 20 minutes, the kinase reaction was initiated by addition of [³³P]-ATP (specific activity 10 μCi/μl) and the mixture was incubated at room temperature for 2 hours. The reaction was then stopped by spotting the reaction mixture on strips of phosphocellulose P81 paper. Following washing, the radioactivity of the P81 paper was measured and kinase activity data were expressed as the percent remaining kinase activity in test samples compared to vehicle reactions. IC₅₀ values and curve fits were obtained using Prism (GraphPad Software).

### IL-1β Release Assay

Approximately 1.5 million THP-1 cells were plated in each well of a 6-well TC plate and incubated with 40 nM PMA in RPMI (10% FBS, 1% Penstrep) for 24 hours. The media was then removed and cells were rested in RPMI (10% FBS, 1% Penstrep) for 24 hours after which time the media was removed and cells were pre-treated for 2 hours with various concentrations of compounds of interest (typically serial 3-fold dilution in RPMI +5% FBS, concentrations ranging from 1 μM to 0.5 nM) in RPMI (5% FBS). The media was again removed and cells were incubated with 250 ng/mL LPS and compounds of interest (concentrations as above) in RMPI (5% FBS) for 2 hours. The media was removed for a last time and cells were incubated with 20 μM nigericin and compounds of interest (concentrations as above) in Opti-MEM for 30 minutes. Cell media was then collected and the amount of cleaved IL-1β was determined using a JESS instrument (Protein Simple) and standard protocols. Cleaved 1-PD antibody was obtained from Cell Signaling (catalogue #83186S) and was used at 1:20 dilution in antibody diluent 2. Protein Simple 1× anti-Rabbit HRP secondary antibody was used along with Protein Simple luminol and peroxide for chemiluminescent detection. Primary antibody incubation time was increased from 30 minutes to 60 minutes.

### Abbreviations:

° C. (degree Celsius); ¹H NMR (proton Nuclear Magnetic Resonance); ACN (acetonitrile); Boc (tert-butyloxycarbonyl); DCM (dichloromethane); DIPEA (N,N-diisopropylethylamine); DMAP (4-dimethylaminopyridine); DMF (N,N-dimethylformamide); DMSO-d₆ (deuterated dimethylsulfoxide); eq (equivalent), EtOAc (ethyl acetate); g (gram); h (hour); HPLC (High Performance Liquid Chromatography); LCMS (Liquid Chromatography Mass Spectrometry), LDA (lithium diisopropylamide). M (molar); MeOH (methanol); mg (milligram); min (minute); mL (milliliter); mmol (millimole); n-BuOH (1-butanol); Pd(PPh₃)₄ (palladium-tetrakis(triphenylphosphine)); PdCl₂(dppf) ([1,1′-bis(diphenylphosphino)ferrocene]palladium(II) dichloride); NBS(N-bromosuccinimide); NCS (N-chlorosuccinimide); Pd₂(dba)₃ (tris(dibenzylideneacetone)dipalladium(0)); RP-HPLC (Reverse-Phase High Performance Liquid Chromatography); SEM-Cl (2-(trimethylsilyl)ethoxymethyl chloride); T3P (1-propanephosphonic anhydride); TBAF (tetra-n-butylammonium fluoride), TBDMS (tert-butyldimethylsilyl), TFA (trifluoroacetic acid); THF (tetrahydrofuran); TLC (Thin Layer Chromatography); UPLC (Ultra Performance Liquid Chromatography); XPhos (2-dicyclohexylphosphino-2′,4′,6′-triisopropylbiphenyl)

### PREPARATION OF SYNTHETIC Intermediate S

### Intermediate IA-A1

### 1-cyclopropyl-3-iodo-1H-pyrazolo[3,4-d]pyrimidin-4-amine

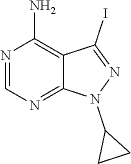 [SMILES: Nc1ncnc2c1c(I)nn2C1CC1]

Copper (II) acetate (0.348 g, 1.916 mmol), 2,2′-bipyridine (0.299 g, 1.916 mmol), and sodium bicarbonate (0.322 g, 3.830 mmol) were added to a stirred solution of 3-iodo-1H-pyrazolo[3,4-d]pyrimidin-4-amine (0.500 g, 1.916 mmol) and cyclopropylboronic acid (0.329 g, 3.830 mmol) in dichloroethane (10 mL). The resulting mixture was stirred at 70° C. under oxygen atmosphere for 12 h. Following completion of the reaction (as indicated by TLC), the reaction mixture was filtered through a pad of of diatomaceous earth which was then rinsed with DCM (20 mL×2). The combined filtrates were washed with water (20 mL) and brine (25 mL), the organic layer separated, dried over Na₂SO₄, filtered, and concentrated under reduced pressure to yield crude material which was purified by flash chromatography (silica gel 230-400 mesh, eluting with EtOAc:petroleum ether 20:80), giving the title compound as an off-white solid (0.24 g, 36% yield). ¹H NMR (400 MHz, DMSO-d₆) δ=8.21 (s, 1H), 3.74-3.79 (m, 1H), 1.11-1.15 (m, 2H), 1.04-1.09 (m, 2H); LCMS: 301.8 [M+H].

### Intermediate IA-A2

### 3-iodo-1-isopropyl-1H-pyrazolo[3,4-d]pyrimidin-4-amine

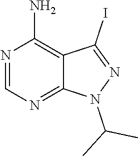

Cs₂CO₃ (12.38 g, 38.31 mmol) and 2-iodopropane (3.60 g, 21.16 mmol) were added to a stirred solution of 3-iodo-1H-pyrazolo[3,4-d]pyrimidin-4-amine (5.00 g, 19.15 mmol) in DMF (25 mL) in a sealed 25 mL tube. The reaction mixture was stirred at 90° C. for 16 h and, following completion of the reaction (as indicated by TLC), was poured into crushed ice (50 g) and stirred for 15 min. The resulting solid was filtered, washed with water (2 mL), and dried to afford the title compound as an off-white solid (3.25 g, 56% yield). ¹H NMR (400 MHz, DMSO-d₆) δ=8.18 (s, 1H), 4.93-4.99 (m, 1H), 1.42 (d, J=6.8 Hz, 6H); LCMS: 303.8 [M+H].

### Intermediates IA-A3 to IA-A12

The following intermediates were prepared via a similar procedure described for Intermediate Ia-A2, replacing 2-iodopropane with the appropriate reagent (alkyl halide or tosylate shown below:

| | | | ¹HNMR (400 MHz, | |
|---|---|---|---|---|
| Intermediate | Structure | Reagent | DMSO-d₆) | LCMS |
| Ia-A3 | 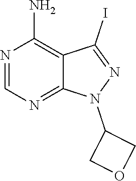 [SMILES: Nc1ncnc2c1c(I)nn2C1COC1] | 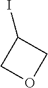 | δ = 8.21 (s, 1H), 5.89-5.93 (m, 1H), 4.93-5.00 (m, 4H). | 318.0 [M + H] |
| Ia-A4 | 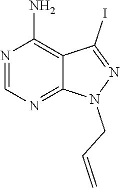 | 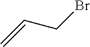 | δ = 8.21 (s, 1H), 5.93-6.03 (m, 1H), 5.16-5.20 (m, 1H), 5.03-5.09 (m, 1H), 4.89- 4.91 (m, 2H). | 301.8 [M + H] |
| Ia-A5 | 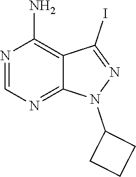 [SMILES: Nc1ncnc2c1c(I)nn2C1CCC1] | 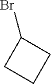 | δ = 8.19 (s, 1H), 5.20-5.26 (m, 1H), 2.28-2.73 (m, 4H), 1.84-1.86 (m, 2H). | 315.8 [M + H] |
| Ia-A6 | 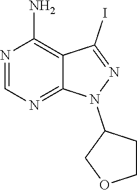 [SMILES: Nc1ncnc2c1c(I)nn2C1CCOC1] | 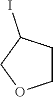 | δ = 8.21 (s, 1H), 5.38-5.44 (m, 1H), 4.00-4.07 (m, 2H), 3.83-3.88 (m, 2H), 2.32- 2.43 (m, 2H). | 332.0 [M + H] |
| Ia-A7 | 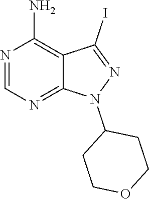 [SMILES: Nc1ncnc2c1c(I)nn2C1CCOCC1] | 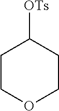 [SMILES: Cc1ccc(S(=O)(=O)OC2CCOCC2)cc1] | δ = 8.20 (s, 1H), 4.82-4.89 (m, 1H), 3.96-4.00 (m, 2H), 3.48-3.54 (m, 2H), 2.05- 2.16 (m, 2H), 1.82-1.86 (m, 2H). | 345.9 [M + H] |
| Ia-A8 | 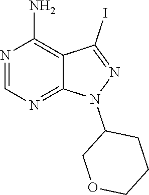 [SMILES: Nc1ncnc2c1c(I)nn2C1CCCOC1] | 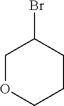 | δ = 8.21 (s, 1H), 4.65-4.71 (m, 1H), 3.85-3.92 (m, 2H), 3.40-3.44 (m, 2H), 2.08- 2.20 (m, 2H), 1.74-1.79 (m, 2H). | 346.0 [M + H] |
| Ia-A9 | 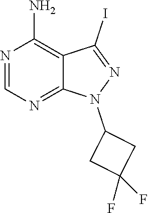 [SMILES: Nc1ncnc2c1c(I)nn2C1CC(F)(F)C1] | 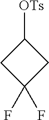 [SMILES: Cc1ccc(S(=O)(=O)OC2CC(F)(F)C2)cc1] | δ = 8.27 (s, 1H), 5.19-5.21 (m, 1H), 3.35 (bs, 4H). | 352.0 [M + H] |
| Ia-A10 | 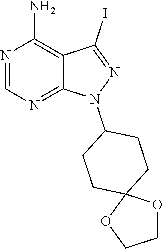 [SMILES: Nc1ncnc2c1c(I)nn2C1CCC2(CC1)OCCO2] | 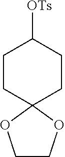 [SMILES: Cc1ccc(S(=O)(=O)OC2CCC3(CC2)OCCO3)cc1] | Crude material was taken as such to the next step. | 402.0 [M + H] |
| Ia-A11 | 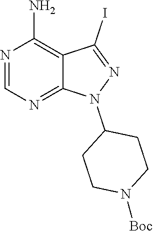 [SMILES: CC(C)(C)OC(=O)N1CCC(n2nc(I)c3c(N)ncnc32)CC1] | 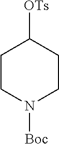 [SMILES: Cc1ccc(S(=O)(=O)OC2CCN(C(=O)OC(C)(C)C)CC2)cc1] | Crude material was taken as such to the next step. | 444.8 [M + H] |
| Ia-A12 | 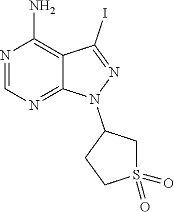 [SMILES: Nc1ncnc2c1c(I)nn2C1CCS(=O)(=O)C1] | 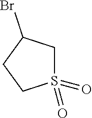 | δ = 8.24 (s, 1H), 5.59-5.67 (m, 1H), 3.70-3.75 (m, 2H), 3.44-3.52 (m, 2H), 2.56- 2.64 (m, 2H). | 379.7 [M + H] |

### Intermediate IA-A13

### 1-(4-amino-3-iodo-1H-pyrazolo[3,4-d]pyrimidin-1-yl)-2-methylpropan-2-ol

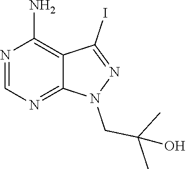 [SMILES: CC(C)(O)Cn1nc(I)c2c(N)ncnc21]

NaH₂PO₄ (0.044 g, 0.372 mmol) was added to a mixture of 3-iodo-1H-pyrazolo[3,4-d]pyrimidin-4-amine (0.100 g, 0.380 mmol), 2,2-dimethyloxirane (0.055 g, 0.760 mmol), and K₂CO₃ (0.050 g, 0.372 mmol) in acetonitrile (3 mL) and water (1 mL) and the resulting solution subjected to microwave irradiation at 150° C. for 1 h. After completion of the reaction (monitored by TLC), the solvents were removed under reduced pressure to yield crude material which was purified by flash chromatography (silica gel 230-400 mesh, eluting with EtOAc:petroleum ether 25:75), giving the title compound as a pale brown solid (0.064 g, 51% yield). ¹H NMR (400 MHz, DMSO-d₆) δ=8.20 (s, 1H), 4.19 (s, 2H), 1.09 (s, 6H); LCMS: 334.0 [M+H].

### Intermediate IA-A14

### 3-iodo-1-(pyridin-4-yl)-1H-pyrazolo[3,4-d]pyrimidin-4-amine

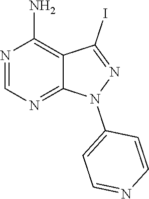 [SMILES: Nc1ncnc2c1c(I)nn2-c1ccncc1]

The title compound was prepared via a similar procedure described for Intermediate Ia-A1, starting from 3-iodo-11H-pyrazolo[3,4-d]pyrimidin-4-amine (1.00 g, 3.83 mmol) and pyridin-4-ylboronic acid (0.94 g, 7.66 mmol), and was obtained as a pale brown solid (0.27 g, 21% yield). LCMS: 338.8 [M+H].

### Intermediate IA-A15

### 3-iodo-1-methyl-1H-pyrazolo[3,4-d]pyrimidin-4-amine

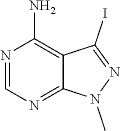

Cs₂CO₃ (0.780 g, 2.394 mmol) and methyl iodide (0.138 mL, 2.203 mmol) were added to a solution of 3-iodo-1H-pyrazolo[3,4-d]pyrimidin-4-amine (0.500 g, 1.916 mmol) in DMF (3 mL) at 0° C. in a sealed 25 mL tube. The reaction mixture was stirred at 25° C. for 1 h and, following completion of the reaction (as indicated by TLC), was poured into crushed ice (50 g) and stirred for 30 min. The resulting solid was filtered, washed with water (2.5 mL), and dried to afford the title compound as a yellow solid (0.380 g, 67% yield). ¹H NMR (400 MHz, DMSO-d₆) δ=8.22 (s, 1H), 6.79 (s, 2H), 3.89 (s, 3H); LCMS: 276.0 [M+H].

### Intermediate IA-A16

### 1-cyclopropyl-3-iodo-1H-pyrazolo[4,3-c]pyridin-4-amine

### Step 1: Synthesis of 4-chloro-3-iodo-1H-pyrazolo[4,3-c]pyridine

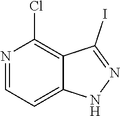 [SMILES: Clc1nccc2[nH]nc(I)c12]

KOH (1.320 g, 23.0 mmol) and iodine (1.620 g, 12.8 mmol) were added to a solution of 4-chloro-1H-pyrazolo[4,3-c]pyridine (1.000 g, 6.4 mmol) in dioxane (10 mL) and the resulting mixture was stirred at 75° C. for 4 h. Following completion of the reaction (as indicated by TLC), the reaction mixture was filtered over a pad of of diatomaceous earth and the filtrate was concentrated under reduced pressure to give crude material which was purified by reverse-phase column chromatography, giving the title compound as a white solid (0.633 g, 63% yield). ¹H NMR (400 MHz, DMSO-d₆) δ=14.12 (bs, 1H), 8.14 (d, J=6.0 Hz, 1H), 7.66 (d, J=5.6 Hz, 1H); LCMS: 279.9 [M+H].

### Step 2: 4-chloro-1-cyclopropyl-3-iodo-1H-pyrazolo[4,3-c]pyridine

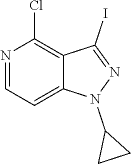 [SMILES: Clc1nccc2c1c(I)nn2C1CC1]

The title compound was prepared via a similar procedure described for Intermediate Ia-A1, starting from 4-chloro-3-iodo-1H-pyrazolo[4,3-c]pyridine (0.630 g, 2.20 mmol) and cyclopropylboronic acid (0.329 g, 3.83 mmol), and was obtained as a white solid (0.430 g, 60% yield). ¹H NMR (400 MHz, DMSO-d₆) δ=8 21 (d, J=6.0 Hz, 1H), 7.81 (d, J=5.6 Hz, 1H), 3.84-3.89 (m, 1H), 1.14-1.17 (m, 4H); LCMS: 319.7 [M+H].

### Step 3: Synthesis of 1-cyclopropyl-3-iodo-1H-pyrazolo[4,3-c]pyridin-4-amine

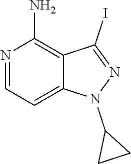 [SMILES: Nc1nccc2c1c(I)nn2C1CC1]

A mixture of 4-chloro-1-cyclopropyl-3-iodo-1 μl-pyrazolo[4,3-c]pyridine (0.20 g) and aqueous ammonium hydroxide (25% in water, 8 mL) was subjected to microwave irradiation at 150° C. for 2 h. Following completion of the reaction (as indicated by TLC), the reaction mixture was concentrated under reduced pressure to yield the title compound as an off-white solid (0.19 g, quantitative yield). ¹H NMR (400 MHz, DMSO-d₆) δ=8.11 (s, 1H), 7.38 (s, 1H), 6.68 (bs, 2H), 3.48-3.54 (m, 1H), 0.97-0.99 (m, 4H); LCMS: 301.0 [M+H].

### Intermediate IA-B1

### 3-(4-amino-3-fluorophenyl)-1-cyclopropyl-1H-pyrazolo[3,4-d]pyrimidin-4-amine

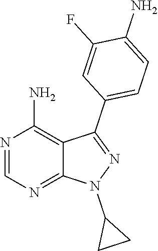 [SMILES: Nc1ccc(-c2nn(C3CC3)c3ncnc(N)c23)cc1F]

A mixture of 1-cyclopropyl-3-iodo-1H-pyrazolo[3,4-d]pyrimidin-4-amine (Ia-A1, 0.500 g, 1.66 mmol), 2-fluoro-4-(4,4,5,5-tetramethyl-1,3,2-dioxaborolan-2-yl)aniline (0.433 g, 1.82 mmol), and K₂CO₃ (0.688 g, 4.98 mmol) in 1,4-dioxane (25 mL) and water (2.5 mL) was purged with N₂ for 10 min. Pd(PPh₃)₄ (0.092 g, 0.08 mmol) was then added and the reaction mixture was stirred at 100° C. for 16 h. After completion of the reaction IC (monitored by TLC), the mixture was filtered through a pad of diatomaceous earth which was then rinsed with EtOAc (2×10 mL). The combined filtrates were concentrated under reduced pressure to yield crude material which was purified by flash chromatography (silica gel 230-400 mesh, eluting with MeOH:DCM 98:2), giving the title compound as a yellow solid (0.46 g, 98% yield). ¹H NMR (400 MHz, DMSO-d₆) δ=8.23 (s, 1H), 7.15-7.24 (m, 2H), 6.87-6.91 (m, 1H), 5.47 (bs, 2H), 3.80-3.84 (m, 1H), 1.18-1.19 (m, 2H), 1.05-1.08 (m, 2H); LCMS: 285.0 [M+H].

### Intermediate IA-B2

### 3-(4-aminophenyl)-1-isopropyl-1H-pyrazolo[3,4-d]pyrimidin-4-amine

### Step 1: Synthesis of 1-isopropyl-3-(4-nitrophenyl)-1H-pyrazolo[3,4-d]pyrimidin-4-amine

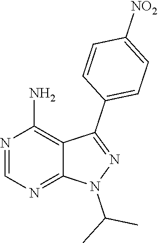 [SMILES: CC(C)n1nc(-c2ccc([N+](=O)[O-])cc2)c2c(N)ncnc21]

The title compound was prepared via a similar procedure described for Intermediate Ia-B1, starting from 3-iodo-1-isopropyl-1H-pyrazolo[3,4-d]pyrimidin-4-amine (Ia-A2, 1.887 g, 6.23 mmol) and (4-nitrophenyl)boronic acid (1.56 g, 9.34 mmol), and was obtained as a yellow solid (1.242 g, 67% yield). ¹H NMR (400 MHz, DMSO-d₆) δ=8.38-8.40 (m, 2H), 8.28 (s, 1H), 7.92-7.95 (m, 2H), 5.07-5.14 (m, 1H), 1.51 (d, J=6.8 Hz, 6H); LCMS: 299.1 [M+H].

### Step 2: Synthesis of 3-(4-aminophenyl)-1-isopropyl-1H-pyrazolo[3,4-d]pyrimidin-4-amine

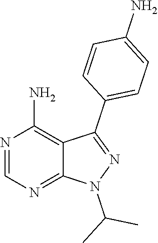 [SMILES: CC(C)n1nc(-c2ccc(N)cc2)c2c(N)ncnc21]

Iron powder (2.320 g, 41.60 mmol) and ammonium chloride (2.220 g, 41.60 mmol) were added to a stirred solution of 1-isopropyl-3-(4-nitrophenyl)-1H-pyrazolo[3,4-d]pyrimidin-4-amine (1.242 g, 4.16 mmol) in ethanol (50 mL) and water (20 mL) and the resulting mixture was heated to 80° C. for 3 h. After completion of the reaction (monitored by TLC), the reaction mixture was filtered through a pad of of diatomaceous earth which was then rinsed with EtOAc (2-25 mL). The combined filtrates were concentrated under reduced pressure, the residue was dissolved in EtOAc (100 mL), washed with brine (25 mL), dried over Na₂SO₄, filtered, and evaporated under reduced pressure to give the title compound as a pale yellow solid (1.042 g, quantitative yield) which was taken forward without further purification.

### Intermediate IA-B3

### 3-(4-amino-3-fluorophenyl)-1-isopropyl-1H-pyrazolo[3,4-d]pyrimidin-4-amine

### Step 1: Synthesis of 3-(3-fluoro-4-nitrophenyl)-1-isopropyl-1H-pyrazolo[3,4-d]pyrimidin-4-amine

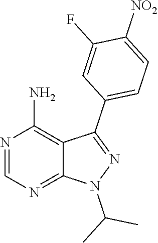 [SMILES: CC(C)n1nc(-c2ccc([N+](=O)[O-])c(F)c2)c2c(N)ncnc21]

The title compound was prepared via a similar procedure described for Intermediate Ia-B1, starting from 3-iodo-1-isopropyl-1H-pyrazolo[3,4-d]pyrimidin-4-amine, (Ia-A2, 0.10 g, 0.32 mmol) and (3-fluoro-4-nitrophenyl)boronic acid (0.71 g, 0.39 mmol), and was obtained as a yellow solid (0.07 g, 67% yield). LCMS: 315.1 [M−H].

### Step 2: Synthesis of 3-(4-amino-3-fluorophenyl)-1-isopropyl-1H-pyrazolo[3,4-d]pyridin-4-amine

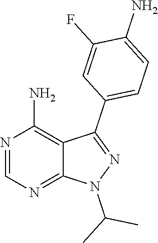 [SMILES: CC(C)n1nc(-c2ccc(N)c(F)c2)c2c(N)ncnc21]

The title compound was prepared via a similar procedure described for step 2 of Intermediate Ia-B2, starting from 3-(3-fluoro-4-nitrophenyl)-1-isopropyl-11-pyrazolo[3,4-d]pyrimidin-4-amine (0.07 g, 0.22 mmol) and Fe/NH₄Cl, and was obtained as a pale-yellow solid (0.09 g, quantitative yield) which was taken forward without further purification. LCMS: 287.1 [M+H].

### Intermediate IA-B4

### 3-(4-aminophenyl)-1-(oxetan-3-yl)-1H-pyrazolo[3,4-d]pyrimidin-4-amine

### Step 1: Synthesis of 3-(4-nitrophenyl)-1-(oxetan-3-yl)-1H-pyrazolo[3,4-d]pyrimidin-4-amine

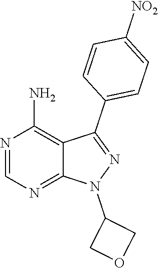 [SMILES: Nc1ncnc2c1c(-c1ccc([N+](=O)[O-])cc1)nn2C1COC1]

The title compound was prepared via a similar procedure described for Intermediate Ia-B1, starting from 3-iodo-1-(oxetan-3-yl)-1H-pyrazolo[3,4-d]pyrimidin-4-amine (Ia-A3, 0.800 g, 2.522 mmol) and (4-nitrophenyl)boronic acid (0.632 g, 3.78 mmol), and was obtained as a yellow solid (0.596 g, 76% yield). ¹H NMR (400 MHz, DMSO-d₆) δ=8.41-8.43 (m, 2H), 8.30 (s, 1H), 7.99-8.01 (m, 2H), 6.05-6.08 (m, 1H), 4.97-5.12 (m, 4H); LCMS: 311.0 [M−H].

### Step 2: Synthesis of 3-(4-aminophenyl)-1-(oxetan-3-yl)-1H-pyrazolo[3,4-d]pyrimidin-4-amine

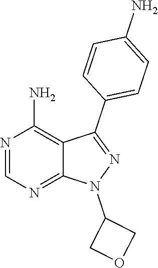 [SMILES: Nc1ccc(-c2nn(C3COC3)c3ncnc(N)c23)cc1]

The title compound was prepared via a similar procedure described for step 2 of Intermediate Ia-B2, starting from 3-(4-nitrophenyl)-1-(oxetan-3-yl)-1H-pyrazolo[3,4-d]pyrimidin-4-amine (0.596 g, 1.91 mmol) and Fe/NH₄Cl, and was obtained as a pale yellow solid (0.42 g, quantitative yield) which was taken forward without further purification. LCMS: 283.0 [M+H].

### Intermediate IA-B5

### 3-(4-amino-3-fluorophenyl)-1-(oxetan-3-yl)-1H-pyrazolo[3,4-d]pyrimidin-4-amine

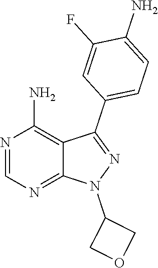 [SMILES: Nc1ccc(-c2nn(C3COC3)c3ncnc(N)c23)cc1F]

The title compound was prepared via a similar procedure described for Intermediate Ia-B1, starting from 3-iodo-1-(oxetan-3-yl)-1H-pyrazolo[3,4-d]pyrimidin-4-amine (Ia-A3, 0.110 g, 0.346 mmol) and 2-fluoro-4-(4,4,5,5-tetramethyl-1,3,2-dioxaborolan-2-yl)aniline (0.099 g, 0.420 mmol), and was obtained as a pale yellow gum (0.077 g, 74% yield). LCMS: 317.1 [M+H].

### Intermediate IA-B6

### 1-allyl-3-(4-amino-3-fluorophenyl)-1H-pyrazolo[3,4-d]pyrimidin-4-amine

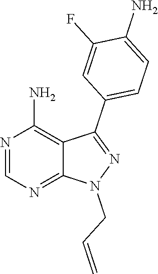 [SMILES: C=CCn1nc(-c2ccc(N)c(F)c2)c2c(N)ncnc21]

The title compound was prepared via a similar procedure described for Intermediate Ia-B1, starting from 1-allyl-3-iodo-1H-pyrazolo[3,4-d]pyrimidin-4-amine (Ia-A4, 0.15 g, 0.49 mmol) and 2-fluoro-4-(4,4,5,5-tetramethyl-1,3,2-dioxaborolan-2-yl)aniline (0.13 g, 0.54 mmol), and was obtained as a yellow gum (0.13 g, 92% yield). LCMS: 285.0 [M+H].

### Intermediate IA-B7

### 3-(4-aminophenyl)-1-cyclopropyl-1H-pyrazolo[3,4-d]pyrimidin-4-amine

### Step 1: Synthesis of 1-cyclopropyl-3-(4-nitrophenyl)-1H-pyrazolo[3,4-d]pyrimidin-4-amine

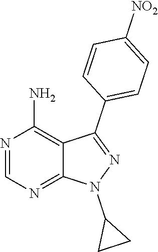 [SMILES: Nc1ncnc2c1c(-c1ccc([N+](=O)[O-])cc1)nn2C1CC1]

The title compound was prepared via a similar procedure described for Intermediate Ia-B1, starting from 1-cyclopropyl-3-iodo-1H-pyrazolo[3,4-d]pyrimidin-4-amine (Ia-A1 0.110 g, 0.36 mmol) and (4-nitrophenyl)boronic acid (0.067 g, 0.40 mmol), and was obtained as a pale yellow solid (0.060 g, 56% yield). ¹H NMR (400 MHz, DMSO-d₆) δ=8.37 (d, J=88 Hz, 2H), 8.30 (s, 1H), 7.91 (d, J=8.8 Hz, 2H), 3.91-3.94 (m, 1H), 1.23-1.24 (m, 2H), 1.11-1.14 (m, 2H); LCMS: 297.0 [M+H].

### Step 2: Synthesis of 3-(4-aminophenyl)-1-cyclopropyl-1H-pyrazolo[3,4-d]pyrimidin-4-amine

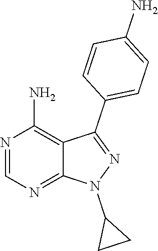 [SMILES: Nc1ccc(-c2nn(C3CC3)c3ncnc(N)c23)cc1]

The title compound was prepared via a similar procedure described for step 2 of Intermediate Ia-B2, starting from 1-cyclopropyl-3-(4-nitrophenyl)-1H-pyrazolo[3,4-d]pyrimidin-4-amine (0.060 g, 0.2 mmol) and Fe/NH₄Cl, and was obtained as a pale yellow solid (0.047 g, 89% yield) which was taken forward without further purification. LCMS: 266.9 [M+H].

### Intermediate IA-B8

### 3-(4-aminophenyl)-1-cyclobutyl-1H-pyrazolo[3,4-d]pyrimidin-4-amine

### Step 1: Synthesis of 1-cyclobutyl-3-(4-nitrophenyl)-1H-pyrazolo[3,4-d]pyrimidin-4-amine

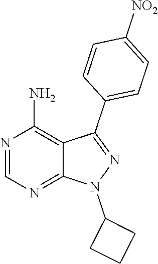 [SMILES: Nc1ncnc2c1c(-c1ccc([N+](=O)[O-])cc1)nn2C1CCC1]

The title compound was prepared via a similar procedure described for Intermediate Ia-B1, starting from 1-cyclobutyl-3-iodo-1H-pyrazolo[3,4-d]pyrimidin-4-amine (Ia-A5, 0.150 g, 0.47 mmol) and (4-nitrophenyl)boronic acid (0.087 g, 0.52 mmol), and was obtained as as pale yellow solid (0.160 g, quantitative yield). ¹H NMR (400 MHz, DMSO-d₆) δ=8.34-8.41 (m, 2H), 8.28 (s, 1H), 7.94-7.97 (m, 2H), 5.32-5.43 (m, 1H), 2.65-2.75 (m, 2H), 1.86-1.94 (m, 4H); LCMS: 311.2 [M+H].

### Step 2: Synthesis of 3-(4-aminophenyl)-1-cyclobutyl-1H-pyrazolo[3,4-d]pyrimidin-4-amine

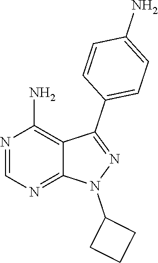 [SMILES: Nc1ccc(-c2nn(C3CCC3)c3ncnc(N)c23)cc1]

The title compound was prepared via a similar procedure described for step 2 of Intermediate Ia-B2, starting from 1-cyclobutyl-3-(4-nitrophenyl)-1H-pyrazolo[3,4-d]pyrimidin-4-amine (0.16 g, 0.51 mmol) and Fe/NH₄Cl, and was obtained as a pale yellow solid (0.140 g, quantitative yield) which was taken forward without further purification. LCMS: 281.0 [M+H].

### Intermediate IA-B9

### 3-(4-amino-3-fluorophenyl)-1-cyclobutyl-1H-pyrazolo[3,4-d]pyrimidin-4-amine

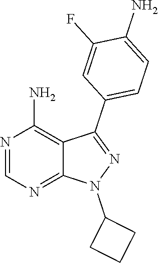 [SMILES: Nc1ccc(-c2nn(C3CCC3)c3ncnc(N)c23)cc1F]

The title compound was prepared via a similar procedure described for Intermediate Ia-B1, starting from 1-cyclobutyl-3-iodo-1H-pyrazolo[3,4-d]pyrimidin-4-amine (Ia-A5, 0.250 g, 0.793 mmol) and 2-fluoro-4-(4,4,5,5-tetramethyl-1,3,2-dioxaborolan-2-yl)aniline (0.225 g, 0.952 mmol), and was obtained as a pale yellow solid (0.100 g, 32% yield). LCMS: 299.1 [M+H].

### Intermediate IA-B10

### 3-(4-aminophenyl)-1-(tetrahydrofuran-3-yl)-1H-pyrazolo[3,4-d]pyrimidin-4-amine

### Step 1: Synthesis of 3-(4-nitrophenyl)-1-(tetrahydrofuran-3-yl)-1H-pyrazolo[3,4-d]pyrimidin-4-amine

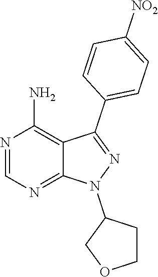 [SMILES: Nc1ncnc2c1c(-c1ccc([N+](=O)[O-])cc1)nn2C1CCOC1]

The title compound was prepared via a similar procedure described for Intermediate Ia-B1, starting from 3-iodo-1-(tetrahydrofuran-3-yl)-1H-pyrazolo[3,4-d]pyrimidin-4-amine (Ia-A6, 0.165 g, 0.49 mmol) and (4-nitrophenyl)boronic acid (0.091 g, 0.54 mmol), and was obtained as pale yellow solid (0.086 g, 53% yield). ¹H NMR (400 MHz, DMSO-d₆) δ=8.38-8.40 (m, 2H), 8.30 (s, 1H), 7.93-7.95 (m, 2H), 5.52-5.58 (m, 1H), 4.06-4.15 (m, 2H), 3.88-3.99 (m, 2H), 2.50-2.51 (m, 2H); LCMS: 327.2 [M+H].

### Step 2: Synthesis of 3-(4-aminophenyl)-1-(tetrahydrofuran-3-yl)-1H-pyrazolo[3,4-d]pyrimidin-4-amine

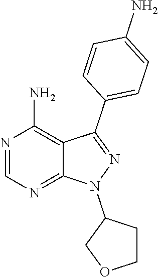 [SMILES: Nc1ccc(-c2nn(C3CCOC3)c3ncnc(N)c23)cc1]

The title compound was prepared via a similar procedure described for step 2 of Intermediate Ia-B2, starting from 3-(4-nitrophenyl)-1-(tetrahydrofuran-3-yl)-1H-pyrazolo[3,4-d]pyrimidin-4-amine (0.115 g, 0.35 mmol) and Fe/NH₄Cl, and was obtained as a pale yellow solid (0.083 g, 80% yield) which was taken forward without further purification. ¹H NMR (400 MHz, DMSO-d₆) δ=8.22 (s, 1H), 7.33 (dd, J=2.0, 6.6 Hz, 2H), 6.71 (dd, J=2.0, 6.4 Hz, 2H), 5.48 (bs, 2H), 5.44-5.47 (m, 1H), 4.04-4.12 (m, 2H), 3.86-3.94 (m, 2H), 2.34-2.41 (m, 2H); LCMS: 296.9 [M+H].

### Intermediate IA-B11

### 3-(4-amino-3-fluorophenyl)-1-(tetrahydrofuran-3-yl)-1H-pyrazolo[3,4-d]pyrimidin-4-amine

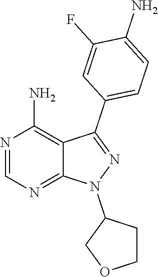 [SMILES: Nc1ccc(-c2nn(C3CCOC3)c3ncnc(N)c23)cc1F]

The title compound was prepared via a similar procedure described for Intermediate Ia-B1, starting from 3-iodo-1-(tetrahydrofuran-3-yl)-1H-pyrazolo[3,4-d]pyrimidin-4-amine (Ia-A6, 0.351 g, 1.00 mmol) and 2-fluoro-4-(4,4,5,5-tetramethyl-1,3,2-dioxaborolan-2-yl)aniline (0.301 g, 1.27 mmol), and was obtained as a pale yellow solid (0.200 g, 60% yield). ¹H NMR (400 MHz, DMSO-d₆) δ=8 23 (s, 1H), 7.24-7.28 (m, 1H), 7.18-7.21 (m, 1H), 6.88-6.92 (m, 1H), 5.46-5.50 (m, 3H), 4.04-4.12 (m, 2H), 3.87-3.94 (m, 2H), 2.33-2.41 (m, 2H); LCMS: 315.1 [M+H].

### Intermediate IA-B12

### 3-(4-aminophenyl)-1-(tetrahydro-2H-pyran-4-yl)-1H-pyrazolo[3,4-d]pyrimidin-4-amine

### Step 1: Synthesis of 3-(4-nitrophenyl)-1-(tetrahydro-2H-pyran-4-yl)-1H-pyrazolo[3,4-d]pyrimidin-4-amine

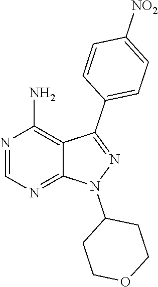 [SMILES: Nc1ncnc2c1c(-c1ccc([N+](=O)[O-])cc1)nn2C1CCOCC1]

The title compound was prepared via a similar procedure described for Intermediate Ia-B1, starting from 3-iodo-1-(tetrahydrofuran-3-yl)-1H-pyrazolo[3,4-d]pyrimidin-4-amine (Ia-A7, 0.094 g, 0.273 mmol) and (4-nitrophenyl)boronic acid (0.055 g, 0.328 mmol), and was obtained as a pale yellow solid (0.078 g, 84% yield). ¹H NMR (400 MHz, DMSO-d₆) δ=8.37-8.40 (m, 2H), 8.29 (s, 1H), 7.93-7.96 (m, 2H), 4.96-5.02 (m, 1H), 4.00-4.04 (m, 2H), 3.54-3.59 (m, 2H), 2.16-2.24 (m, 2H), 1.89-1.94 (m, 2H); LCMS: 340.9 [M+H].

### Step 2: Synthesis of 3-(4-aminophenyl)-1-(tetrahydro-2H-pyran-4-yl)-1H-pyrazolo[3,4-d]pyrimidin-4-amide

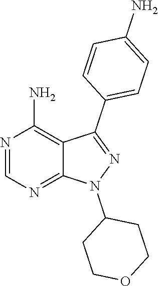 [SMILES: Nc1ccc(-c2nn(C3CCOCC3)c3ncnc(N)c23)cc1]

The title compound was prepared via a similar procedure described for step 2 of Intermediate Ia-B2, starting from 3-(4-nitrophenyl)-1-(tetrahydro-2H-pyran-4-yl)-1H-pyrazolo[3,4-d]pyrimidin-4-amine (0.078 g, 0.23 mmol) and Fe/NH₄Cl, and was obtained as a pale yellow solid (0.052 g, 73% yield) which was taken forward without further purification. LCMS: 311.1 [M+H].

### Intermediate IA-B13

### 3-(4-amino-3-fluorophenyl)-1-(tetrahydro-2H-pyran-3-yl)-1H-pyrazolo[3,4-d]pyrimidin-4-amine

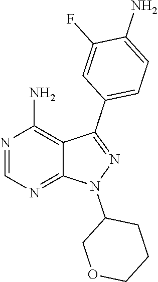 [SMILES: Nc1ccc(-c2nn(C3CCCOC3)c3ncnc(N)c23)cc1F]

The title compound was prepared via a similar procedure described for Intermediate Ia-B1, starting from 3-iodo-1-(tetrahydro-2H-pyran-3-yl)-1H-pyrazolo[3,4-d]pyrimidin-4-amine (Ia-A8, 0.050 g, 0.14 mmol) and 2-fluoro-4-(4,4,5,5-tetramethyl-1,3,2-dioxaborolan-2-yl)aniline (0.040 g, 0.17 mmol), and was obtained as a yellow gum (0.0.15 g, 32% yield) LCMS: 329.2 [M+H].

### Intermediate IA-B14

### 3-(4-amino-3-fluorophenyl)-1-(3,3-difluorocyclobutyl)-1H-pyrazolo[3,4-d]pyrimidin-4-amine

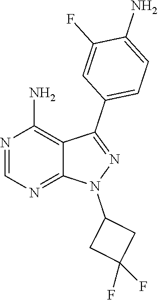 [SMILES: Nc1ccc(-c2nn(C3CC(F)(F)C3)c3ncnc(N)c23)cc1F]

The title compound was prepared via a similar procedure described for Intermediate Ia-B1, starting from 1-(3,3-difluorocyclobutyl)-3-iodo-1H-pyrazolo[3,4-d]pyrimidin-4-amine (Ia-A9, 0.100 g, 0.285 mmol) and 2-fluoro-4-(4,4,5,5-tetramethyl-1,3,2-dioxaborolan-2-yl)aniline (0.810 g, 0.341 mmol), and was obtained as a pale yellow solid (0.088 g, 93% yield). LCMS: 334.9 [M+H].

### Intermediate IA-B15

### 4-(4-amino-3-(4-amino-3-fluorophenyl)-1H-pyrazolo[3,4-d]pyrimidin-1-yl)cyclohexan-1-ol

### Step 1: Synthesis of 4-(4-amino-3-iodo-1H-pyrazolo[3,4-d]pyrimidin-1-yl)cyclohexan-1-ol

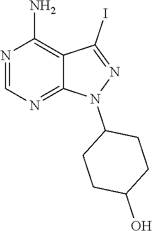 [SMILES: Nc1ncnc2c1c(I)nn2C1CCC(O)CC1]

Aqueous HCl (1.5 N, 5 mL) was added to a solution of 3-iodo-1-(1,4-dioxaspiro[4.5]decan-8-yl)-1H-pyrazolo[3,4-d]pyrimidin-4-amine (Ia-A10, 0.313 g, 0.780 mmol) and the resulting mixture was stirred at room temperature for 12 h. After completion of the reaction (monitored by UPLC), the solvent was removed under reduced pressure to yield as residue (0.232 g, LCMS: 358.0 [M+H]) which was dissolved in THF (5 mL). The resulting solution was cooled to 0° C., NaBH₄ (0.050 g, 1.322 mmol) was added, and the mixture was stirred at room temperature for 1 h. After completion of the reaction (monitored by UPLC), aqueous HCl (1.5 N, 5 mL) was added and the mixture was extracted with EtOAc (2×10 mL). The combined organic layers were dried over Na₂SO₄, filtered, and concentrated under reduced pressure to yield the title product as a pale brown gum (0.196 g) which was taken forward without further purification. LCMS: 359.8 [M+H].

### Step 2: Synthesis of 4-(4-amino-3-(4-amino-3-fluorophenyl)-1H-pyrazolo[3,4-d]pyrimidin-1-yl)cyclohexan-1-ol

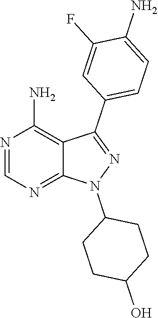 [SMILES: Nc1ccc(-c2nn(C3CCC(O)CC3)c3ncnc(N)c23)cc1F]

The title compound was prepared via a similar procedure described for Intermediate Ia-B1, starting from 4-(4-amino-3-iodo-1H-pyrazolo[3,4-d]pyrimidin-1-yl)cyclohexan-1-ol (0.196 g, 0.550 mmol) and 2-fluoro-4-(4,4,5,5-tetramethyl-1,3,2-dioxaborolan-2-yl)aniline (0.160 g, 0.655 mmol), and was obtained as a yellow gum (0.120 g, 64% yield). LCMS: 343.0 [M+H].

### Intermediate IA-B16

### 3-(4-amino-3-fluorophenyl)-1-(1-(oxetan-3-yl)piperidin-4-yl)-1H-pyrazolo[3,4-d]pyrimidin-4-amine

### Step 1: Synthesis of tert-butyl 4-(4-amino-3-(3-fluoro-4-nitrophenyl)-1H-pyrazolo[3,4-d]pyrimidin-1-yl)piperidine-1-carboxylate

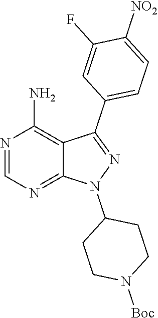 [SMILES: CC(C)(C)OC(=O)N1CCC(n2nc(-c3ccc([N+](=O)[O-])c(F)c3)c3c(N)ncnc32)CC1]

The title compound was prepared via a similar procedure described for Intermediate Ia-B1, starting from tert-butyl 4-(4-amino-3-iodo-1H-pyrazolo[3,4-d]pyrimidin-1-yl)piperidine-1-carboxylate (Ia-A11, 0.10 g, 0.23 mmol) and (3-fluoro-4-nitrophenyl)boronic acid (0.05 g, 0.27 mmol), and was obtained as a yellow solid (0.04 g, 40% yield). LCMS: 458.1 [M−H].

### Step 2: Synthesis of 3-(3-fluoro-4-nitrophenyl)-1-(piperidin-4-yl)-1H-pyrazolo[3,4-d]pyridin-4-amine

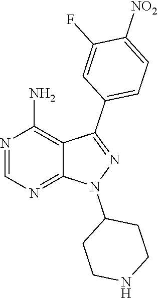 [SMILES: Nc1ncnc2c1c(-c1ccc([N+](=O)[O-])c(F)c1)nn2C1CCNCC1]

TFA (0.5 mL) was added to a solution of tert-butyl 4-(4-amino-3-(3-fluoro-4-nitrophenyl)-1H-pyrazolo[3,4-d]pyrimidin-1-yl)piperidine-1-carboxylate (100 mg, 0.218 mmol) in dry DCM (5 mL) and the resulting mixture was stirred at room temperature for 2 h. Following completion of the reaction (as indicated by UPLC), the solvent was evaporated under reduced pressure to yield the title product as a pale brown gum (110 mg) which was taken forward without further purification. LCMS: 358.1 [M−H].

### Step 3: Synthesis of 3-(3-fluoro-4-nitrophenyl)-1-(1-(oxetan-3-yl)piperidin-4-yl)-1H-pyrazolo[3,4-d]pyrimidin-4-amine

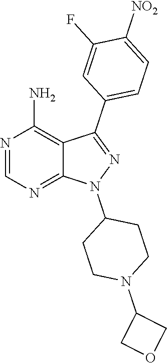 [SMILES: Nc1ncnc2c1c(-c1ccc([N+](=O)[O-])c(F)c1)nn2C1CCN(C2COC2)CC1]

Oxetan-3-one (0.020 g, 0.277 mmol) and glacial acetic acid (catalytic amount) were added to a solution of 3-(3-fluoro-4-nitrophenyl)-1-(piperidin-4-yl)-1H-pyrazolo[3,4-d]pyrimidin-4-amine (0.100 g, 0.280 mmol) in DCM (5 mL) and the resulting mixture was stirred at room temperature for 4 h. Sodium triacetoxyborohydride (0.178 g, 0.840 mmol) was then added and the resulting mixture was stirred at room temperature for 12 h. Following completion of the reaction (as indicated by UPLC), the solution was diluted with DCM (5 mL) and washed with aqueous 10% NaHCO₃ (5 mL) and brine (5 mL). The organic layer was dried over Na₂SO₄, filtered, and the solvents were evaporated under reduced pressure to yield the title product (110 mg) which was taken forward without further purification. LCMS: 414.2 [M−H].

### Step 4: Synthesis of 3-(4-amino-3-fluorophenyl)-1-(1-(oxetan-3-yl)piperidin-4-yl)-1H-pyrazolo[3,4-d]pyrimidin-4-amine

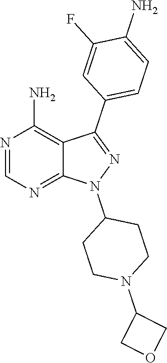 [SMILES: Nc1ccc(-c2nn(C3CCN(C4COC4)CC3)c3ncnc(N)c23)cc1F]

Iron powder (0.135 g, 2.417 mmol) and ammonium chloride (0.142 g, 2.655 mmol) were added to a solution of 3-(3-fluoro-4-nitrophenyl)-1-(1-(oxetan-3-yl)piperidin-4-yl)-1H-pyrazolo[3,4-d]pyrimidin-4-amine (0.110 g, 0.266 mmol) in ethanol (5 mL) and water (2 mL) and the resulting mixture was stirred at 80° C. for 2 h. Following completion of the reaction (as indicated by TLC), the reaction mixture was filtered through a pad of of diatomaceous earth which was then rinsed with EtOAc (5 mL×2). The combined filtrates were concentrated under reduced pressure to give a residue which was dissolved in EtOAC (10 mL) then washed with brine (5 mL). The organic layer separated, dried over Na₂SO₄, filtered, and concentrated under reduced pressure to yield the title compound as an off-white solid (0.05 g, 50% yield) which was taken forward without further purification. LCMS: 383.9 [M+H].

### Intermediate IA-B17

### 3-(4-amino-3-(4-amino-3-fluorophenyl)-1H-pyrazolo[3,4-d]pyrimidin-1-yl)tetrahydrothiophene 1,1-dioxide

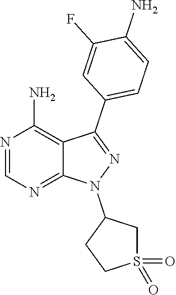 [SMILES: Nc1ccc(-c2nn(C3CCS(=O)(=O)C3)c3ncnc(N)c23)cc1F]

The title compound was prepared via a similar procedure described for Intermediate Ia-B, starting from 3-(4-amino-3-iodo-11H-pyrazolo[3,4-d]pyrimidin-1-yl)tetrahydrothiophene 1,1-dioxide (Ia-A12, 0.08 g, 0.21 mmol) and 2-fluoro-4-(4,4,5,5-tetramethyl-1,3,2-dioxaborolan-2-yl)aniline (0.06 g, 0.25 mmol), and was obtained as a yellow gum (0.03 g, 40% yield). ¹H NMR (400 MHz, DMSO-d₆) δ=8 26 (s, 1H), 7.27-7.31 (m, 1H), 7.20-7.23 (m, 1H), 6.88-6.93 (m, 1H), 5.69 (bs, 2H), 5.53 (bs, 2H), 3.72-3.78 (m, 2H), 3.51-3.56 (m, 2H), 2.61-2.70 (m, 2H); LCMS: 362.8 [M+H].

### Intermediate IA-B18

### 1-(4-amino-3-(4-aminophenyl)-1H-pyrazolo[3,4-d]pyrimidin-1-yl)-2-methylpropan-2-ol

### Step 1: Synthesis of 1-(4-amino-3-(4-nitrophenyl)-1H-pyrazolo[3,4-d]pyrimidin-1-yl-2-methylpropan-2-ol

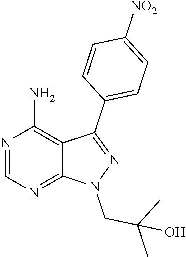 [SMILES: CC(C)(O)Cn1nc(-c2ccc([N+](=O)[O-])cc2)c2c(N)ncnc21]

The title compound was prepared via a similar procedure described for Intermediate Ia-B1, starting from 1-(4-amino-3-iodo-1H-pyrazolo[3,4-d]pyrimidin-1-yl)-2-methylpropan-2-ol (Ia-A13, 0.067 g, 0.201 mmol) and (4-nitrophenyl)boronic acid (0.054 g, 0.302 mmol), and was obtained as a yellow solid (0.059 g, 90% yield). ¹H NMR (400 MHz, DMSO-d₆) δ=8 40 (d, J=7.6 Hz, 2H), 8.30 (s, 1H), 7.94 (d, J=6.8 Hz, 2H), 4.32 (bs, 2H), 1.16 (bs, 6H); LCMS: 328.9 [M−H].

### Step 2: Synthesis of 1-(4-amino-3-(4-aminophenyl)-1H-pyrazolo[3,4-d]pyrimidin-1-yl)-2-methylpropan-2-ol

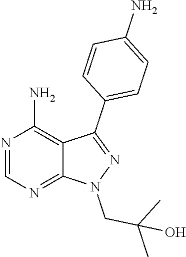 [SMILES: CC(C)(O)Cn1nc(-c2ccc(N)cc2)c2c(N)ncnc21]

The title compound was prepared via a similar procedure described for step 2 of Intermediate Ia-B2, starting from 1-(4-amino-3-(4-nitrophenyl)-1H-pyrazolo[3,4-d]pyrimidin-1-yl)-2-methylpropan-2-ol (0.072 g, 0.219 mmol) and Fe/NH₄Cl, and was obtained as a pale yellow solid (0.07 g, quantitative yield) which was taken forward without further purification. LCMS: 299.0 [M+H].

### Intermediate IA-B19

### 1-(4-amino-3-(4-amino-3-fluorophenyl)-1H-pyrazolo[3,4-d]pyrimidin-1-yl)-2-methylpropan-2-ol

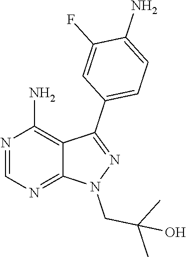 [SMILES: CC(C)(O)Cn1nc(-c2ccc(N)c(F)c2)c2c(N)ncnc21]

The title compound was prepared via a similar procedure described for Intermediate Ia-B1, starting from 1-(4-amino-3-iodo-1H-pyrazolo[3,4-d]pyrimidin-1-yl)-2-methylpropan-2-ol (Ia-A13, 0.110 g, 0.330 mmol) and 2-fluoro-4-(4,4,5,5-tetramethyl-1,3,2-dioxaborolan-2-yl)aniline (0.094 g, 0.396 mmol), and was obtained as a pale yellow solid (0.077 g, 66% yield); LCMS: 317.1 [M+H].

### Intermediate IA-B20

### 3-(4-amino-3-fluorophenyl)-1-(pyridin-4-yl)-1H-pyrazolo[3,4-d]pyrimidin-4-amine

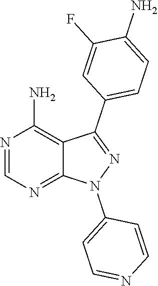 [SMILES: Nc1ccc(-c2nn(-c3ccncc3)c3ncnc(N)c23)cc1F]

The title compound was prepared via a similar procedure described for Intermediate Ia-B1, starting from 3-iodo-1-(pyridin-4-yl)-1H-pyrazolo[3,4-d]pyrimidin-4-amine (Ia-A14, 0.250 g, 0.73 mmol) and 2-fluoro-4-(4,4,5,5-tetramethyl-1,3,2-dioxaborolan-2-yl)aniline (0.193 g, 0.81 mmol), and was obtained as a yellow gum (0.140 g, 59% yield). LCMS: 321.9 [M+H].

### Intermediate IA-B21

### 3-(4-amino-3-chlorophenyl)-1-isopropyl-1H-pyrazolo[3,4-d]pyrimidin-4-amine

### Step 1: Synthesis of 3-(3-chloro-4-nitrophenyl)-1-isopropyl-1H-pyrazolo[3,4-d]pyrimidin-4-amine

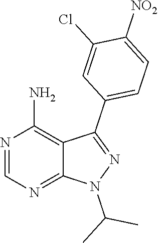 [SMILES: CC(C)n1nc(-c2ccc([N+](=O)[O-])c(Cl)c2)c2c(N)ncnc21]

The title compound was prepared via a similar procedure described for Intermediate Ia-B1, starting from 3-iodo-1-isopropyl-1H-pyrazolo[3,4-d]pyrimidin-4-amine (Ia-A2, 0.115 g, 0.379 mmol) and 2-(3-chloro-4-nitrophenyl)-4,4,5,5-tetramethyl-1,3,2-dioxaborolane (0.129 g, 0.455 mmol), and was obtained as an off-white solid (0.083 g, 66% yield). ¹H NMR (400 MHz, DMSO-d₆) δ=8.28 (s, 1H), 8.21-8.23 (m, 1H), 7.95-7.95 (m, 1H), 7.82-7.85 (m, 1H), 5.07-5.13 (m, 1H), 1.51 (d, J=6.4 Hz, 6H); LCMS: 332.9 [M+H].

### Step 2: Synthesis of 3-(4-amino-3-chlorophenyl)-1-isopropyl-1H-pyrazolo[3,4-d]pyrimidin-4-amine

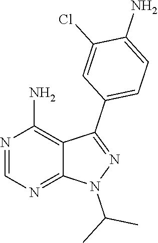 [SMILES: CC(C)n1nc(-c2ccc(N)c(Cl)c2)c2c(N)ncnc21]

The title compound was prepared via a similar procedure described for step 2 of Intermediate Ia-B2, starting from 3-(3-chloro-4-nitrophenyl)-1-isopropyl-1H-pyrazolo[3,4-d]pyrimidin-4-amine (0.083 g, 0.25 mmol) and Fe/NH₄Cl, and was obtained as a pale yellow solid (0.900 g, quantitative yield) which was taken forward without further purification. LCMS: 302.9 [M+H].

### Intermediate IA-B22

### 3-(4-amino-3-chlorophenyl)-1-methyl-1H-pyrazolo[3,4-d]pyrimidin-4-amine

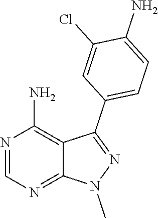 [SMILES: Cn1nc(-c2ccc(N)c(Cl)c2)c2c(N)ncnc21]

The title compound was prepared via a similar procedure described for Intermediate Ia-B1, starting from 3-iodo-1-methyl-1H-pyrazolo[3,4-d]pyrimidin-4-amine (Ia-A15, 0.100 g, 0.364 mmol) and 2-chloro-4-(4,4,5,5-tetramethyl-1,3,2-dioxaborolan-2-yl)aniline (0.092 g, 0.364 mmol), and was obtained as a yellow solid (0.094 g, 94% yield). ¹H NMR (400 MHz, DMSO-d₆) δ=8.23 (s, 1H), 7.45-7.46 (m, 1H), 7.31-7.33 (m, 1H), 6.91-6.93 (m, 1H), 5.68 (bs, 2H), 3.91 (s, 3H), LCMS: 275.0[M+H].

### Intermediate IA-B23

### 3-(4-amino-3-fluorophenyl)-1-methyl-1H-pyrazolo[3,4-d]pyrimidin-4-amine

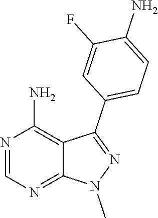 [SMILES: Cn1nc(-c2ccc(N)c(F)c2)c2c(N)ncnc21]

The title compound was prepared via a similar procedure described for Intermediate Ia-B1, starting from 3-iodo-1-methyl-1H-pyrazolo[3,4-d]pyrimidin-4-amine (Ia-B15, 0.110 g, 0.400 mmol) and 2-fluoro-4-(4,4,5,5-tetramethyl-1,3,2-dioxaborolan-2-yl)aniline (0.104 g, 0.440 mmol), and was obtained as a brown gum (0.130 g, 97% yield). LCMS: 259.1[M+H].

### Intermediate IA-B24

### 3-(4-amino-3-fluorophenyl)-1-cyclopropyl-1H-pyrazolo[4,3-c]pyridin-4-amine

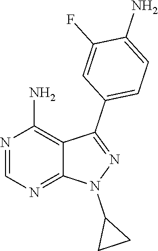 [SMILES: Nc1ccc(-c2nn(C3CC3)c3ncnc(N)c23)cc1F]

The title compound was prepared via a similar procedure described for Intermediate Ia-B1, starting from 1-cyclopropyl-3-iodo-1H-pyrazolo[4,3-c]pyridin-4-amine (Ia-A16, 0.190 g, 0.63 mmol) and 2-fluoro-4-(4,4,5,5-tetramethyl-1,3,2-dioxaborolan-2-yl)aniline (0.180 g, 0.75 mmol), and was obtained as a yellow gum (0.070 g, 39% yield). ¹H NMR (400 MHz, DMSO-d₆) δ=7.75-7.76 (m, 1H), 7.21-7.24 (m, 1H), 7.14-7.16 ((m, 2H), 6.85-6.87 (m, 1H), 5.78 (bs, 2H), 5.46 (bs, 2H), 3.67-3.69 (m, 1H), 1.09-1.10 (m, 4H); LCMS: 317.1 [M+H].

### Intermediate IA-B25

### 3-(4-aminophenyl)-1-cyclopropyl-1H-pyrazolo[4,3-c]pyridin-4-amine

### Step 2: Synthesis of 3-(4-cyclopropyl-3-(4-nitrophenyl)-1H-pyrazolo[4,3-c]pyridin-4-amine

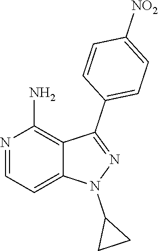 [SMILES: Nc1nccc2c1c(-c1ccc([N+](=O)[O-])cc1)nn2C1CC1]

The title compound was prepared via a similar procedure described for Intermediate Ia-B1, starting from 1-cyclopropyl-3-iodo-11-pyrazolo[4,3-c]pyridin-4-amine (Ia-A16, 0.190 g, 0.63 mmol) and (4-nitrophenyl)boronic acid (0.126 g, 0.75 mmol), and was obtained as a pale yellow solid (0.090 g, 50% yield). ¹H NMR (400 MHz, DMSO-d₆) δ=8.36-8.39 (m, 2H), 7.93-7.96 (m, 2H), 7.84 (d, J=6.0 Hz, 1H), 6.95 (d, J=6.0 Hz, 1H), 5.98 (bs, 2H), 3.78-3.81 (m, 1H), 1.13-1.16 (m, 4H); LCMS: 296.1 [M+H].

### Step 2: Synthesis of 3-(4-aminophenyl)-1-cyclopropyl-1H-pyrazolo[4,3-c]pyridin-4-amine

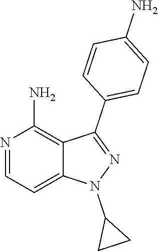 [SMILES: Nc1ccc(-c2nn(C3CC3)c3ccnc(N)c23)cc1]

The title compound was prepared via a similar procedure described for step 2 of Intermediate Ia-B2, starting from 1-cyclopropyl-3-(4-nitrophenyl)-1H-pyrazolo[4,3-c]pyridin-4-amine (0.093 g, 031 mmol) and Fe/NH₄Cl, and was obtained as a pale yellow solid (0.052 g, 63% yield) which was taken forward without further purification. LCMS: 266.0 [M+H].

### General Procedure for the Synthesis of Carbamates Intermediates Ia-C

Pyridine (1.2 eq) and phenyl chloroformate (1.5 eq) were added to a solution of amine (1.0 eq) in THF (10 vol) at 0° C. The reaction mixture was allowed to warm to 25° C. and was stirred for 12 h. After completion of the reaction (monitored by TLC), the mixture was diluted with EtOAc (10 mL) and washed with brine (5 mL). The organic layer was dried over Na₂SO₄, filtered, and concentrated under reduced pressure to yield crude material which was purified by flash chromatography (silica gel 230-400 mesh, eluting with EtOAc:petroleum ether 10:90 to 20:80), giving the desired carbamate. The following carbamates were prepared using the above general procedure.

| Carbamates | Structure | Starting material | ¹H NMR/LCMS |
|---|---|---|---|
| Ia-C1 | 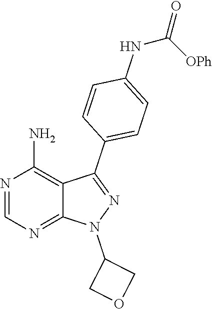 [SMILES: Nc1ncnc2c1c(-c1ccc(NC(=O)Oc3ccccc3)cc1)nn2C1COC1] | 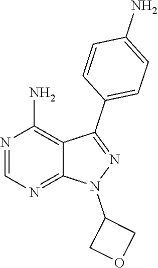 [SMILES: Nc1ccc(-c2nn(C3COC3)c3ncnc(N)c23)cc1] | 403.1 [M + H] |
| Ia-C2 | 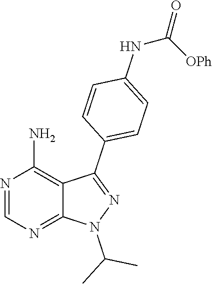 [SMILES: CC(C)n1nc(-c2ccc(NC(=O)Oc3ccccc3)cc2)c2c(N)ncnc21] | 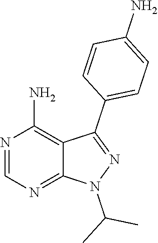 | 389.1 [M + H] |
| Ia-C3 | 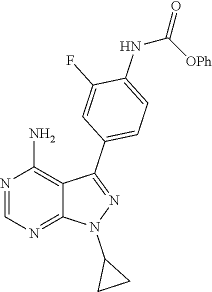 [SMILES: Nc1ncnc2c1c(-c1ccc(NC(=O)Oc3ccccc3)c(F)c1)nn2C1CC1] | 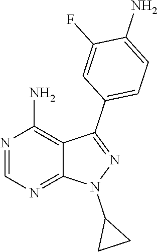 [SMILES: Nc1ccc(-c2nn(C3CC3)c3ncnc(N)c23)cc1F] | 405.2 [M + H] |
| Ia-C4 | 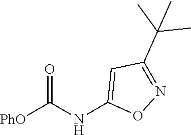 [SMILES: CC(C)(C)c1cc(NC(=O)Oc2ccccc2)on1] | 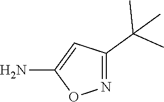 | 261.0 [M + H] |
| Ia-C5 | 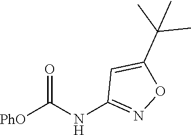 [SMILES: CC(C)(C)c1cc(NC(=O)Oc2ccccc2)no1] | 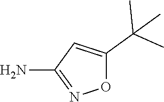 | 261.0 [M + H] |
| Ia-C6 | 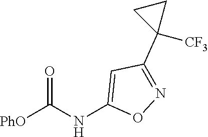 [SMILES: O=C(Nc1cc(C2(C(F)(F)F)CC2)no1)Oc1ccccc1] | 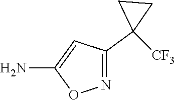 [SMILES: Nc1cc(C2(C(F)(F)F)CC2)no1] | 312.9 [M + H] |
| Ia-C7 | 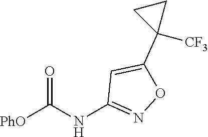 [SMILES: O=C(Nc1cc(C2(C(F)(F)F)CC2)on1)Oc1ccccc1] | 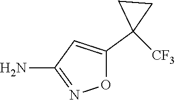 [SMILES: Nc1cc(C2(C(F)(F)F)CC2)on1] | 312.9 [M + H] |
| Ia-C8 | 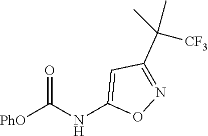 [SMILES: CC(C)(c1cc(NC(=O)Oc2ccccc2)on1)C(F)(F)F] | 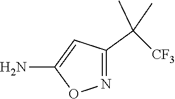 | 315.1 [M + H] |
| Ia-C9 | 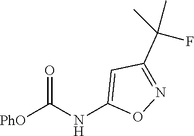 [SMILES: CC(C)(F)c1cc(NC(=O)Oc2ccccc2)on1] | 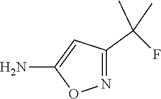 | 265.1 [M + H] |

Note:
Amines used for the synthesis of carbamates are either commercially available or were synthesized using literature procedures as follows:
3-(1-(trifluoromethyl)cyclopropyl)isoxazol-5-amine (precursor to Ia-C6) and 5-(1-(trifluoromethyl)cyclopropyl)isoxazol-3-amine (precursor to Ia-C7) were synthesized from 1-(trifluoromethyl)cyclopropane-1-carboxylic acid methyl ester as reported in Synthesis 2013, 45, 171-173.
3-(1,1,1-trifluoro-2-methylpropan-2-yl)isoxazol-5-amine (precursor to Ia-C8) and 3-(2-fluoropropan-2-yl)isoxazol-5-amine (precursor to Ia-C9) were synthesized from methyl 3,3,3-trifluoro-2,2-dimethylpropanote and methyl 2-fluoro-2-methylpropionate, respectively, followed by the procedure reported in Synthesis 2013, 45, 171-173.

### General Procedure for the Synthesis of Examples Ia-1 Through Ia-B1

Triethylamine (2.0 eq) was added to a mixture of amine Intermediate (Ia-B1 through Ia-B24, 1.0 eq) & carbamate Intermediate (Ia-C1 through Ia-C9, 1.0 eq) in THF (5 mL) and the resulting solution was heated to 60° C. for 12 h.

After completion of the reaction (monitored by TLC), the solvent was evaporated under reduced pressure to yield crude material which was purified by reverse phase preparative HPLC (mass-based, aqueous ammonium acetate:ACN gradient) to give the title compounds.

The following compounds were prepared using the above general procedure.

### Example IA-1

### 1-(4-(4-amino-1-isopropyl-1H-pyrazolo[3,4-d]pyrimidin-3-yl)phenyl)-3-(3-(tert-butyl)isoxazol-5-yl)urea

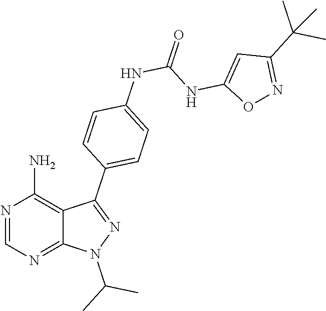 [SMILES: CC(C)n1nc(-c2ccc(NC(=O)Nc3cc(C(C)(C)C)no3)cc2)c2c(N)ncnc21]

The title compound was prepared following the general procedure described above, starting from 3-(4-aminophenyl)-1-isopropyl-1H-pyrazolo[3,4-d]pyrimidin-4-amine (Ia-B2, 0.250 g, 0.93 mmol) and phenyl (3-(tert-butyl)isoxazol-5-yl)carbamate (Ia-C4, 0.242 g, 0.93 mmol), and was obtained as an off-white solid (0.150 g, 37% yield). ¹H NMR (400 MHz, DMSO-d₆) δ 10.15 (bs, 1H), 9.09 (bs, 1H), 8.24 (bs, 1H), 7.55-7.66 (m, 4H), 6.10 (s, 1H), 5.03-5.10 (m, 1H), 1.49 (d, J=6.4 Hz, 6H), 1.27 (s, 9H), LCMS: 435.3 [M+H].

### Example IA-2

### 1-(4-(4-amino-1-(oxetan-3-yl)-1H-pyrazolo[3,4-d]pyrimidin-3-yl)phenyl)-3-(3-(tert-butyl)isoxazol-5-yl)urea

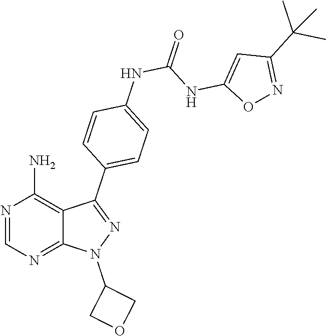 [SMILES: CC(C)(C)c1cc(NC(=O)Nc2ccc(-c3nn(C4COC4)c4ncnc(N)c34)cc2)on1]

The title compound was prepared following the general procedure described for Example Ia-1, starting from 3-(4-aminophenyl)-1-(oxetan-3-yl)-1H-pyrazolo[3,4-d]pyrimidin-4-amine (Ia-B4, 0.210 g, 0.744 mmol) and phenyl (3-(tert-butyl)isoxazol-5-yl)carbamate (Ia-C4, 0.193 g, 0.744 mmol), and was obtained as an off-white solid (0.528 g, 16% yield). ¹H NMR (400 MHz, DMSO-d₆) δ=10.23 (bs, 1H), 9.15 (bs, 1H), 8.31 (s, 1H), 7.74 (s, 4H), 6.16 (s, 1H), 6.06-6.10 (m, 1H), 5.16 (t, J=6.40 Hz, 2H), 5.05 (t, J=6.80 Hz, 2H), 1.33 (s, 9H): LCMS: 449.2[M+H].

### Example IA-3

### 1-(4-(4-amino-1-(oxetan-3-yl)-1H-pyrazolo[3,4-d]pyrimidin-3-yl)phenyl)-3-(5-(tert-butyl)isoxazol-3-yl)urea

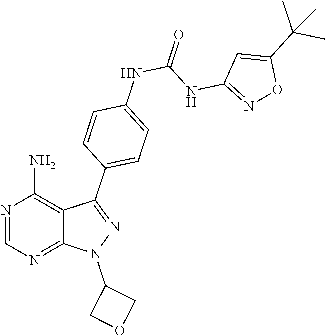 [SMILES: CC(C)(C)c1cc(NC(=O)Nc2ccc(-c3nn(C4COC4)c4ncnc(N)c34)cc2)no1]

The title compound was prepared following the general procedure described for Example Ia-1, starting from 3-(4-aminophenyl)-1-(oxetan-3-yl)-1H-pyrazolo[3,4-d]pyrimidin-4-amine (Ia-B4, 0.070 g, 0.248 mmol) and phenyl (5-(tert-butyl)isoxazol-3-yl)carbamate (Ia-C5, 0.065 g, 0.248 mmol), and was obtained as an off-white solid (0.356 mg, 32% yield). ¹H NMR (400 MHz, DMSO-d₆) δ=9.59 (bs, 1H), 9.07 (bs, 1H), 8.26 (s, 1H), 7.66 (bs, 4H), 6.53 (s, 1H), 5.98-6.04 (m, 1H), 5.09 (t, J=6.4 Hz, 2H), 5.00 (t, J=6.8 Hz, 2H), 1.31 (s, 9H): LCMS: 449.2[M+H].

### Example IA-4

### 1-(4-(4-amino-1-(tetrahydro-2H-pyran-4-yl)-1H-pyrazolo[3,4-d]pyrimidin-3-yl)phenyl)-3-(3-(tert-butyl)isoxazol-5-yl)urea

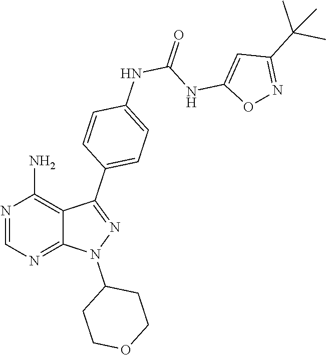 [SMILES: CC(C)(C)c1cc(NC(=O)Nc2ccc(-c3nn(C4CCOCC4)c4ncnc(N)c34)cc2)on1]

The title compound was prepared following the general procedure described for Example Ia-1, starting from 3-(4-aminophenyl)-1-(tetrahydro-2H-pyran-4-yl)-1H-pyrazolo[3,4-d]pyrimidin-4-amine (Ia-B12, 0.05 g, 0.16 mmol) and phenyl (3-(tert-butyl)isoxazol-5-yl)carbamate (Ia-C4, 0.042 g, 0.16 mmol) as an off-white solid (13 mg, 20% yield). ¹H NMR (400 N4 Hz, DMSO-d₆) δ=10.13 (bs, 1H), 9.08 (bs, 1H), 8.25 (s, 1H), 7.61-7.67 (m, 4H), 6.10 (s, 1H), 4.90-4.98 (m, 1H), 4.00-4.03 (m, 2H), 3.53-3.58 (m, 2H), 2.19-2.26 (m, 2H), 1.87-1.91 (m, 2H), 1.27 (s, 9H), LCMS: 477.1[M+H].

### Example IA-5

### 1-(4-(4-amino-1-(tetrahydrofuran-3-yl)-1H-pyrazolo[3,4-d]pyrimidin-3-yl)phenyl)-3-(3-(tert-butyl)isoxazol-5-yl)urea

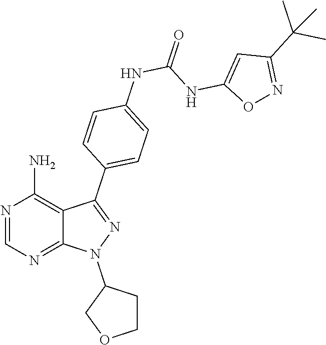 [SMILES: CC(C)(C)c1cc(NC(=O)Nc2ccc(-c3nn(C4CCOC4)c4ncnc(N)c34)cc2)on1]

The title compound was prepared following the general procedure described for Example Ia-1, starting from 3-(4-aminophenyl)-1-(tetrahydrofuran-3-yl)-1H-pyrazolo[3,4-d]pyrimidin-4-amine (Ia-B10, 0.083 g, 0.28 mmol) and phenyl (3-(tert-butyl)isoxazol-5-yl)carbamate (Ia-C4, 0.072 g, 0.28 mmol) as white solid (47.59 mg, 36% yield). ¹H NMR (400 MHz, DMSO-d₆) δ=10.17 (bs, 1H), 9.08 (bs, 1H), 8.26 (s, 1H), 7.60-7.67 (m, 4H), 6.10 (s, 1H), 5.49-5.52 (m, 1H), 4.07-4.14 (m, 2H), 3.89-3.97 (m, 2H), 2.38-2.43 (m, 2H), 1.27 (s, 9H); LCMS: 463.0[M+H].

### Example IA-6

### 1-(4-(4-amino-1-isopropyl-1H-pyrazolo)[3,4-d]pyrimidin-3-yl)-2-chlorophenyl)-3-(3-(tert-butyl)isoxazol-5-yl)urea

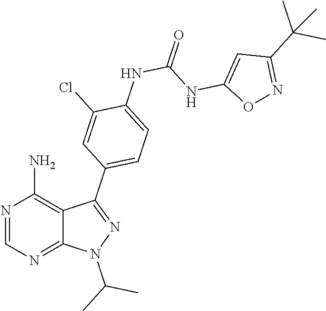 [SMILES: CC(C)n1nc(-c2ccc(NC(=O)Nc3cc(C(C)(C)C)no3)c(Cl)c2)c2c(N)ncnc21]

The title compound was prepared following the general procedure described for Example Ia-1, starting from 3-(4-amino-3-chlorophenyl)-1-isopropyl-1H-pyrazolo[3,4-d]pyrimidin-4-amine (Ia-B21, 0.080 g, 0.264 mmol) and phenyl (3-(tert-butyl)isoxazol-5-yl)carbamate (Ia-C4, 0.067 g, 0.264 mmol), and was obtained as an off-white solid (0.003 g, 3% yield). ¹H NMR (400 MHz, DMSO-d₆) δ=10.85 (bs, 1H), 8.66 (bs, 1H), 8.35 (d, J=8.4 Hz, 1H), 8.25 (s, 1H), 7.72 (d, J=2.0 Hz, 1H), 7.61-7.64 (m, 1H), 6.12 (s, 1H), 5.04-5.10 (m, 1H), 1.50 (d, J=6.8 Hz, 6H), 1.28 (s, 9H); LCMS: 469.1[M+H].

### Example IA-7

### 1-(4-(4-amino-1-cyclobutyl-1H-pyrazolo[3,4-d]pyrimidin-3-yl)phenyl)-3-(3-(tert-butyl)isoxazol-5-yl)urea

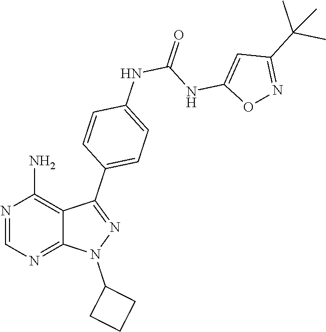 [SMILES: CC(C)(C)c1cc(NC(=O)Nc2ccc(-c3nn(C4CCC4)c4ncnc(N)c34)cc2)on1]

The title compound was prepared following the general procedure described for Example Ia-1, starting from 3-(4-aminophenyl)-1-cyclobutyl-1H-pyrazolo[3,4-d]pyrimidin-4-amine (Ia-B8, 0.140 g, 0.49 mmol) and phenyl (3-(tert-butyl)isoxazol-5-yl)carbamate (Ia-C4, 0.130 g, 0.49 mmol), and was obtained as an off-white solid (0.667 g, 29% yield). ¹H NMR (400 MHz, DMSO-d₆) δ=10.15 (bs, 1H), 9.09 (bs, 1H), 8.24 (s, 1H), 7.62-7.68 (m, 4H), 6.10 (s, 1H), 5.32-5.36 (m, 1H), 2.69-2.74 (m, 2H), 2.39-2.42 (m, 2H), 1.87-1.89 (m, 2H), 1.27 (s, 9H); LCMS: 447.2[M+H].

### Example IA-8

### 1-(4-(4-amino-1-cyclobutyl-1H-pyrazolo[3,4-d]pyrimidin-3-yl)-2-fluorophenyl)-3-(3-(tert-butyl)isoxazol-5-yl)urea

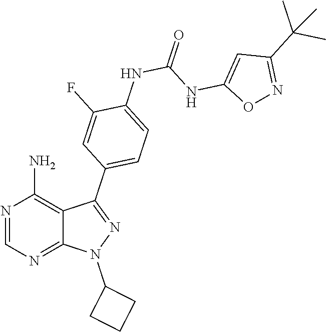 [SMILES: CC(C)(C)c1cc(NC(=O)Nc2ccc(-c3nn(C4CCC4)c4ncnc(N)c34)cc2F)on1]

The title compound was prepared following the general procedure described for Example Ia-1, starting from 3-(4-aminophenyl)-1-cyclobutyl-1H-pyrazolo[3,4-d]pyrimidin-4-amine (Ia-B9, 0.100 g, 0.33 mmol), and phenyl (3-(tert-butyl)isoxazol-5-yl)carbamate (Ia-C4, 0.087 g, 0.33 mmol), and was obtained as a pale yellow solid (0.005 g, 4% yield). ¹H NMR (400 MHz, DMSO-d₆) δ=9.10 (bs, 1H), 8.23-8.28 (m, 2H), 7.47-7.53 (m, 2H), 6.90 (bs, 1H), 6.09 (s, 1H), 5.29-5.37 (m, 1H), 2.67-2.73 (m, 2H), 2.38-2.41 (m, 2H), 1.85-1.89 (m, 2H), 1.26 (s, 9H); LCMS: 465.1 [M+H].

### Example IA-9

### 1-(4-(4-amino-1-(2-hydroxy-2-methylpropyl)-1H-pyrazolo[3,4-d]pyrimidin-3-yl)phenyl)-3-(3-(tert-butyl)isoxazol-5-yl)urea

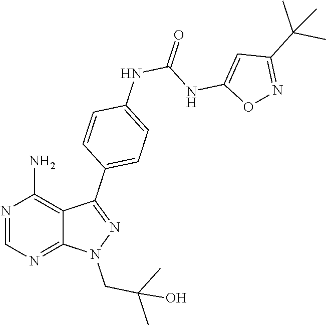 [SMILES: CC(C)(O)Cn1nc(-c2ccc(NC(=O)Nc3cc(C(C)(C)C)no3)cc2)c2c(N)ncnc21]

The title compound was prepared following the general procedure described for Example Ia-1, starting from 1-(4-amino-3-(4-aminophenyl)-1H-pyrazolo[3,4-d]pyrimidin-1-yl)-2-methylpropan-2-ol (Ia-318, 0.119 g, 0.4 mmol) and phenyl (3-(tert-butyl)isoxazol-5-yl)carbamate (Ia-C4, 0.103 g, 0.4 mmol), and was obtained as an off-white solid (0.017 g, 9% yield). ¹H NMR (400 MHz, DMSO-d₆) δ=9.44 (bs, 1H), 8.24 (s, 1H), 7.59-7.68 (m, 4H), 6.07 (s, 1H), 4.81 (bs, 1H), 4.27 (bs, 2H), 1.26 (s, 9H), 1.15 (s, 6H); LCMS 465.1 [M+H].

### Example IA-10

### 1-(4-(4-amino-1-(2-hydroxy-2-methylpropyl)-1H-pyrazolo[3,4-d]pyrimidin-3-yl)-2-fluorophenyl-3-(3-(tert-butyl)isoxazol-5-yl)urea

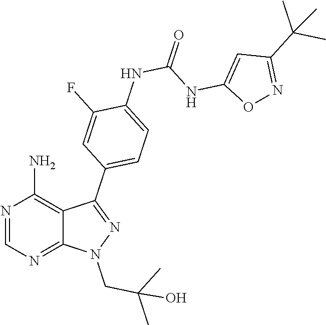 [SMILES: CC(C)(O)Cn1nc(-c2ccc(NC(=O)Nc3cc(C(C)(C)C)no3)c(F)c2)c2c(N)ncnc21]

The title compound was prepared following the general procedure described for Example Ia-1, starting from 1-(4-amino-3-(4-amino-3-fluorophenyl)-1H-1-pyrazolo[3,4-d]pyrimidin-1-yl)-2-methylpropan-2-ol (Ia-B19, 0.077 g, 0.243 mmol) and phenyl (3-(tert-butyl)isoxazol-5-yl)carbamate (Ia-C4, 0.063 g, 0.243 mmol), and was obtained as an off-white solid (0.013 g, 11% yield). ¹H NMR (400 MHz, CD₃OD) δ=8.33-8.37 (m, 1H), 8.29 (s, 1H), 7.53-7.59 (m, 2H), 6.19 (s, 1H), 4.42 (s, 2H), 1.35 (s, 9H), 1.29 (s, 6H); LCMS: 483.3[M+H].

### Example IA-11

### 1-(4-(4-amino-1-isopropyl-1H-pyrazolo[3,4-d]pyrimidin-3-yl)-2-fluorophenyl)-3-(3-(tert-butyl)isoxazol-5-yl)urea

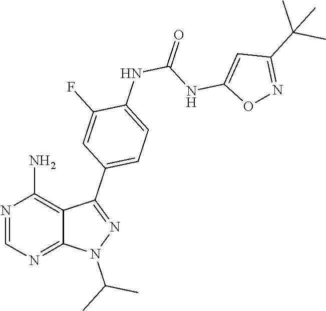 [SMILES: CC(C)n1nc(-c2ccc(NC(=O)Nc3cc(C(C)(C)C)no3)c(F)c2)c2c(N)ncnc21]

The title compound was prepared following the general procedure described for Example Ia-1, starting from 3-(4-amino-3-fluorophenyl)-1-isopropyl-1H-pyrazolo[3,4-d]pyrimidin-4-amine (Ia-B3, 0.200 g, 0.69 mmol) and phenyl (3-(tert-butyl)isoxazol-5-yl)carbamate (Ia-C4, 0.182 g, 0.69 mmol), and was obtained as an off-white solid (0.011 g, 3% yield). ¹H NMR (400 MHz, DMSO-d₆) δ=10.40 (bs, 1H), 8.93 (bs, 1H), 8.24-8.30 (m, 2H), 7.47-7.52 (m, 2H), 6.95 (bs, 2H), 6.11 (s, 1H), 5.03-5.10 (m, 1H), 1.49 (d, J=6.4 Hz, 6H), 1.27 (s, 9H); LCMS: 453.2[M+H].

### Example IA-12

### 1-(4-(4-amino-1-(oxetan-3-yl)-1H-pyrazolo[3,4-d]pyrimidin-3-yl)-2-fluorophenyl)-3-(3-(tert-butyl)isoxazol-5-yl)urea

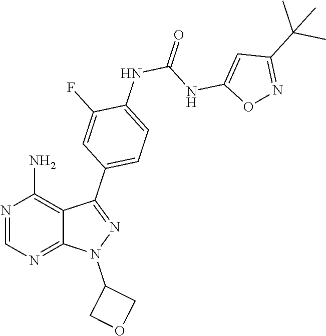 [SMILES: CC(C)(C)c1cc(NC(=O)Nc2ccc(-c3nn(C4COC4)c4ncnc(N)c34)cc2F)on1]

The title compound was prepared following the general procedure described for Example Ia-1, starting from 3-(4-amino-3-fluorophenyl)-1-(oxetan-3-yl)-1H-pyrazolo[3,4-d]pyrimidin-4-amine (Ia-B5, 0.200 g, 0.66 mmol) and phenyl (3-(tert-butyl)isoxazol-5-yl)carbamate (Ia-C4, 0.173 g, 0.66 mmol), and was obtained as an off-white solid (0.027 g, 10% yield). ¹H NMR (400 MHz, DMSO-d₆) δ=8.97 (bs, 1H), 8.29-8.33 (m, 1H), 8.25 (s, 1H), 7.52-7.59 (m, 2H), 6.11 (s, 1H), 5.98-6.05 (m, 1H), 5.01-5.11 (m, 2H), 4.98-4.99 (m, 2H), 1.27 (s, 9H); LCMS: 467.1[M+H].

### Example IA-13

### 1-(4-(4-amino-1-cyclopropyl-1H-pyrazolo[3,4-d]pyrimidin-3-yl)-2-fluorophenyl)-3-(3-(tert-butyl)isoxazol-5-yl)urea

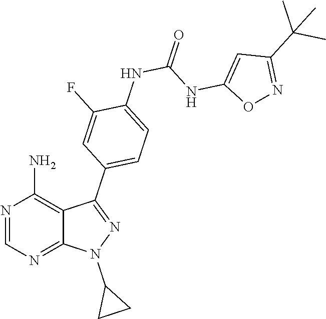 [SMILES: CC(C)(C)c1cc(NC(=O)Nc2ccc(-c3nn(C4CC4)c4ncnc(N)c34)cc2F)on1]

The title compound was prepared following the general procedure described for Example Ia-1, starting from 3-(4-amino-3-fluorophenyl)-1-cyclopropyl-1H-pyrazolo[3,4-d]pyrimidin-4-amine (Ia-B1, 0.150 g, 0.52 mmol) and phenyl (3-(tert-butyl)isoxazol-5-yl)carbamate (Ia-C4, 0.137 g, 0.52 mmol), and was obtained as a white solid (0.035 g, 15% yield). ¹H NMR (400 MHz, DMSO-d₆) δ=10.42 (bs, 1H), 8.98 (bs, 1H), 8.25-8.29 (m, 2H), 7.44-7.50 (m, 2H), 6.95 (bs, 2H), 6.10 (s, 1H), 3.85-3.87 (m, 1H), 1.27 (s, 9H), 1.20-1.23 (m, 2H), 1.08-1.10 (m, 21H); LCMS: 451.2[M+H].

### Example IA-14

### 1-(4-(4-amino-1-(1-(oxetan-3-yl)piperidin-4-yl)-1H-pyrazolo[3,4-d]pyrimidin-3-yl)-2-fluorophenyl)-3-(3-(tert-butyl)isoxazol-5-yl)urea

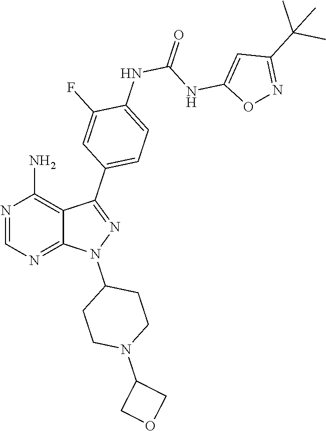 [SMILES: CC(C)(C)c1cc(NC(=O)Nc2ccc(-c3nn(C4CCN(C5COC5)CC4)c4ncnc(N)c34)cc2F)on1]

The title compound was prepared following the general procedure described for Example Ia-1, starting from 3-(4-amino-3-fluorophenyl)-1-(1-(oxetan-3-yl)piperidin-4-yl)-1H-pyrazolo[3,4-d]pyrimidin-4-amine (Ia-B16, 0.050 g, 0.13 mmol) and phenyl (3-(tert-butyl)isoxazol-5-yl)carbamate (Ia-C4, 0.034 g, 0.13 mmol), and was obtained as an off-white solid (0.006 g, 8% yield). ¹H NMR (400 MHz, CD₃OD) δ=8.32-8.36 (m, 1H), 8.27 (s, 1H), 7.51-7.57 (m, 2H), 6.19 (s, 1H), 4.65-4.82 (m, 5H), 3.61-3.64 (m, 1H), 2.98-3.01 (m, 2H), 2.37-2.44 (m, 2H), 2.13-2.19 (m, 2H), 2.04-2.07 (m, 2H), 1.35 (s, 9H); LCMS: 550.0[M+H].

### Example IA-15

### 1-(4-(4-amino-1-(4-hydroxycyclohexyl)-1H-pyrazolo[3,4-d]pyrimidin-3-yl)-2-fluorophenyl-3-(3-(tert-butyl)isoxazol-5-yl)urea

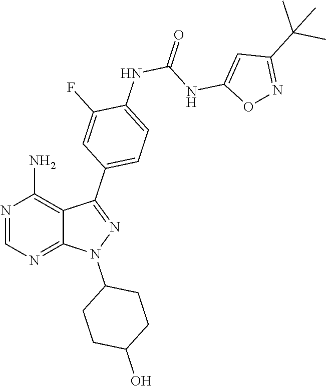 [SMILES: CC(C)(C)c1cc(NC(=O)Nc2ccc(-c3nn(C4CCC(O)CC4)c4ncnc(N)c34)cc2F)on1]

The title compound was prepared following the general procedure described for Example Ia-1, starting from 4-(4-amino-3-(4-amino-3-fluorophenyl)-1H-pyrazolo[3,4-d]pyrimidin-1-yl)cyclohexan-1-ol (Ia-B15, 0.075 g, 0.22 mmol) and phenyl (3-(tert-butyl)isoxazol-5-yl)carbamate (Ia-C4, 0.057 g, 0.22 mmol), and was obtained as an off-white solid (0.007 g, 6% yield). ¹H NMR (400 MHz, CD₃OD) δ=8.32-8.36 (m, 1H), 8.27 (s, 1H), 7.50-7.55 (m, 2H), 6.19 (s, 1H), 4.74-4.80 (m, 1H), 3.72-3.73 (m, 1H), 2.14-2.21 (m, 4H), 2.03-2.06 (m, 2H), 1.55-1.59 (m, 2H), 1.35 (s, 9H); LCMS: 509.2[M+H].

### Example IA-16

### 1-(4-(4-amino-1-(3,3-difluorocyclobutyl)-1H-pyrazolo[3,4-d]pyrimidin-3-yl)-2-fluorophenyl)-3-(3-(tert-butyl)isoxazol-5-yl)urea

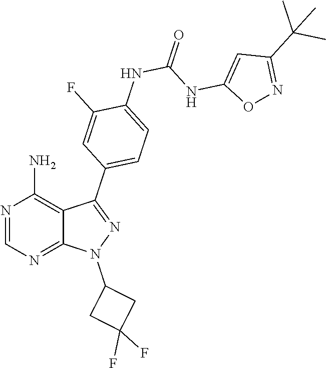 [SMILES: CC(C)(C)c1cc(NC(=O)Nc2ccc(-c3nn(C4CC(F)(F)C4)c4ncnc(N)c34)cc2F)on1]

The title compound was prepared following the general procedure described for Example Ia-1, starting from 3-(4-amino-3-fluorophenyl)-1-(3,3-difluorocyclobutyl)-1H-pyrazolo[3,4-d]pyrimidin-4-amine (Ia-B14, 0.110 g, 0.33 mmol) and phenyl (3-(tert-butyl)isoxazol-5-yl)carbamate (Ia-C4, 0.086 g, 0.33 mmol), and was obtained as an off-white solid (0.020 g, 13% yield). ¹H NMR (400 MHz, DMSO-d₆) δ=9.28 (bs, 1H), 8.26-8.30 (m, 2H), 7.48-7.55 (m, 2H), 7.08 (bs, 1H), 6.07 (s, 1H), 5.28-5.33 (m, 1H), 3.25-3.34 (m, 4H), 1.27 (s, 9H); LCMS: 501.1[M+H].

### Example IA-17

### 1-(4-(4-amino-1-(pyridin-4-yl)-1H-pyrazolo[3,4-d]pyrimidin-3-yl)-2-fluorophenyl)-3-(3-(tert-butyl)isoxazol-5-yl)urea

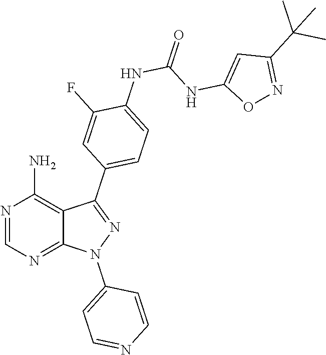 [SMILES: CC(C)(C)c1cc(NC(=O)Nc2ccc(-c3nn(-c4ccncc4)c4ncnc(N)c34)cc2F)on1]

The title compound was prepared following the general procedure described for Example Ia-1, starting from 3-(4-amino-3-fluorophenyl)-1-(pyridin-4-yl)-1H-pyrazolo[3,4-d]pyrimidin-4-amine (Ia-B20, 0.140 g, 0.43 mmol) and phenyl (3-(tert-butyl)isoxazol-5-yl)carbamate (Ia-C4, 0.113 g, 0.43 mmol), and was obtained as an off-white solid (0.006 g, 3% yield). H NMR (400 MHz, DMSO-d₆) δ=10.44 (bs, 1H), 9.00 (bs, 1H), 8.73-8.74 (m, 2H), 8.34-8.47 (m, 4H), 7.59-7.68 (m, 2H), 6.13 (s, 1H), 1.28 (s, 9H), LCMS: 488.2[M+H].

### Example IA-18

### 1-(4-(4-amino-1-(1,1-dioxdotetrahydrothiophen-3-yl)-1H-pyrazolo[3,4-d]pyrimidin-3-yl)-2-fluorophenyl)-3-(3-(tert-butyl)isoxazol-5-yl)urea

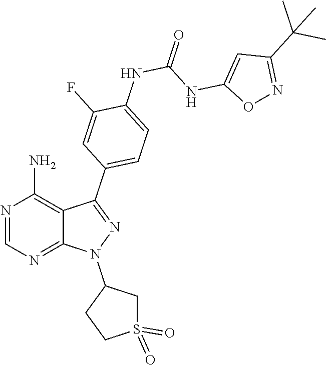 [SMILES: CC(C)(C)c1cc(NC(=O)Nc2ccc(-c3nn(C4CCS(=O)(=O)C4)c4ncnc(N)c34)cc2F)on1]

The title compound was prepared following the general procedure described for Example Ia-1, starting from 3-(4-amino-3-(4-amino-3-fluorophenyl)-1H-pyrazolo[3,4-d]pyrimidin-1-yl)tetrahydrothiophene 1,1-dioxide (Ia-B17, 0.030 g, 0082 mmol) and phenyl (3-(tert-butyl)isoxazol-5-yl)carbamate (Ia-C4, 0.021 g, 0.082 mmol), and was obtained as a white solid (0.003 g, 7% yield). ¹H NMR (400 MHz, CD₃OD) δ=8.31-8.36 (m, 2H), 7.54-7.60 (m, 2H), 6.19 (s, 1H), 5.78-5.80 (m, 1H), 3.58-3.63 (m, 3H), 2.78-2.89 (m, 2H), 1.32 (s, 9H); LCMS: 529.2[M+H].

### Example IA-19

### 1-(4-(4-amino-1-(tetrahydro-2H-pyran-3-yl)-1H-pyrazolo[3,4-d]pyrimidin-3-yl)-2-fluorophenyl)-3-(3-(tert-butyl)isoxazol-5-yl)urea

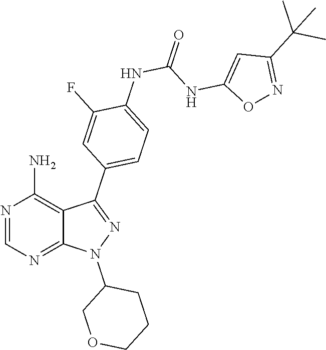 [SMILES: CC(C)(C)c1cc(NC(=O)Nc2ccc(-c3nn(C4CCCOC4)c4ncnc(N)c34)cc2F)on1]

The title compound was prepared following the general procedure described for Example Ia-1, starting from 3-(4-amino-3-fluorophenyl)-1-(tetrahydro-2H-pyran-3-yl)-1H-pyrazolo[3,4-d]pyrimidin-4-amine (Ia-B13, 0.015 g, 0.045 mmol) and phenyl (3-(tert-butyl)isoxazol-5-yl)carbamate (Ia-C4, 0.011 g, 0.045 mmol), and was obtained as an off-white solid (0.002 g, 9% yield). ¹H NMR (400 MHz, CD₃OD) δ=8.54 (s, 1H), 8.35 (d, J=7.2 Hz, 1H), 8.28 (s, 1H), 7.50-7.56 (m, 2H), 6.19 (s, 1H), 4.60 (s, 1H), 3.87-4.05 (m, 3H), 3.50-3.58 (m, 1H), 2.38-2.42 (m, 1H), 2.21-2.23 (m, 1H), 1.92-2.03 (m, 2H), 1.35 (s, 9H); LCMS: 495.1[M+H].

### Example IA-20

### 1-(4-(4-amino-1-cyclopropyl-1H-pyrazolo[3,4-d]pyrimidin-3-yl)phenyl)-3-(3-(tert-butyl)isoxazol-5-yl)urea

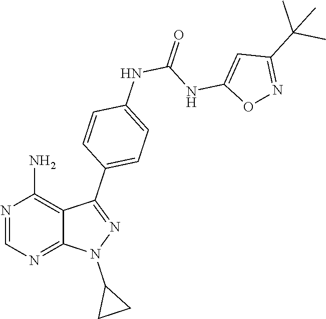 [SMILES: CC(C)(C)c1cc(NC(=O)Nc2ccc(-c3nn(C4CC4)c4ncnc(N)c34)cc2)on1]

The title compound was prepared following the general procedure described for Example Ia-1, starting from 3-(4-aminophenyl)-1-cyclopropyl-1H-pyrazolo[3,4-d]pyrimidin-4-amine (Ia-B7, 0.100 g, 0.38 mmol) and phenyl (3-(tert-butyl)isoxazol-5-yl)carbamate (Ia-C4, 0.100 g, 0.38 mmol), and was obtained as a white solid (0.045 g, 27% yield). ¹H NMR (400 MHz, DMSO-d₆) δ=10.11 (bs, 1H), 9.10 (bs, 1H), 8.26 (s, 1H), 7.58-7.65 (m, 4H), 6.09 (s, 1H), 3.83-3.89 (m, 1H), 1.24 (s, 9H), 1.19-1.23 (m, 2H), 1.06-1.11 (m, 2H), LCMS: 433.2[M+H].

### Example IA-21

### 1-(4-(4-amino-1-cyclopropyl-1H-pyrazolo[3,4-d]pyrimidin-3-yl)-2-fluorophenyl)-3-(4-(1-(Trifluoromethyl)cyclopropyl)isoxazol-5-yl)urea

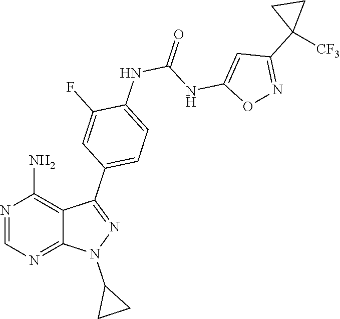 [SMILES: Nc1ncnc2c1c(-c1ccc(NC(=O)Nc3cc(C4(C(F)(F)F)CC4)no3)c(F)c1)nn2C1CC1]

The title compound was prepared following the general procedure described for Example Ia-1, starting from 3-(4-amino-3-fluorophenyl)-1-cyclopropyl-1H-pyrazolo[3,4-d]pyrimidin-4-amine (Ia-B1, 0.100 g, 0.31 mmol) and phenyl (3-(1-(trifluoromethyl)cyclopropyl)isoxazol-5-yl)carbamate (Ia-C6, 0.091 g, 0.31 mmol), and was obtained as an off-white solid (0.020 g, 12% yield). ¹H NMR (400 MHz, DMSO-d₆) δ=10.63 (bs, 1H), 8.92 (bs, 1H), 8.25-8.29 (m, 2H), 7.44-7.51 (m, 2H), 6.87 (bs, 2H), 6.20 (s, 1H), 3.84-3.89 (m, 1H), 1.45-1.76 (m, 2H), 1.36-1.41 (m, 2H), 1.19-1.23 (m, 2H), 1.06-1.11 (m, 2H): LCMS: 503.1[M+H].

### Example IA-22

### 1-(4-(4-amino-1-cyclopropyl-1H-pyrazolo[3,4-d]pyrimidin-3-yl)-2-fluorophenyl)-3-(3-(1,1,1-trifluoro-2-methylpropan-2-yl)isoxazol-5-yl)urea

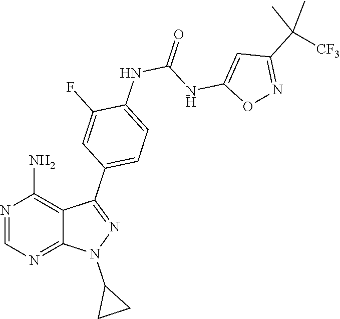 [SMILES: CC(C)(c1cc(NC(=O)Nc2ccc(-c3nn(C4CC4)c4ncnc(N)c34)cc2F)on1)C(F)(F)F]

The title compound was prepared following the general procedure described for Example Ia-1, starting from 3-(4-amino-3-fluorophenyl)-1-cyclopropyl-1H-pyrazolo[3,4-d]pyrimidin-4-amine (Ia-B1, 0.050 g, 0.17 mmol) and phenyl (3-(1,1,1-trifluoro-2-methylpropan-2-yl)isoxazol-5-yl)carbamate (Ia-C8, 0.045 g, 0.17 mmol), and was obtained as an off-white solid (0.004 g, 4% yield). ¹H NMR (400 MHz, DMSO-d₆) δ=10.62 (bs, 1H), 8.96 (bs, 1H), 8.25-8.28 (m, 2H), 7.45-7.52 (m, 2H), 6.99 (bs, 2H), 6.25 (s, 1H), 3.84-3.90 (m, 1H), 1.53 (s, 6H), 1.19-1.23 (m, 2H), 1.10-1.12 (m, 2H), LCMS: 505.1 [M+H].

### Example IA-23

### 1-(4-(4-amino-1-(oxetan-3-yl)-1H-pyrazolo[3,4-d]pyrimidin-3-yl)-2-fluorophenyl)-3-(5-(tert-butyl)isoxazol-3-yl)urea

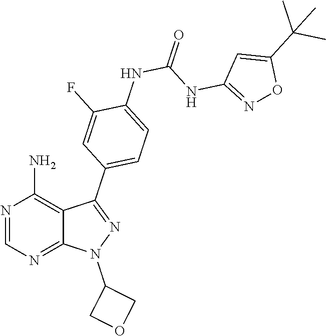 [SMILES: CC(C)(C)c1cc(NC(=O)Nc2ccc(-c3nn(C4COC4)c4ncnc(N)c34)cc2F)no1]

The title compound was prepared following the general procedure described for Example Ia-1, starting from 3-(4-amino-3-fluorophenyl)-1-(oxetan-3-yl)-1H-pyrazolo[3,4-d]pyrimidin-4-amine (Ia-B5, 0.150 g, 0.5 mmol) and phenyl (5-(tert-butyl)isoxazol-3-yl)carbamate (Ia-C5, 0.130 g, 0.5 mmol), and was obtained as an off-white solid (0.015 g, 6% yield). ¹H NMR (400 MHz, DMSO-d₆) δ=10.00 (bs, 1H), 9.09 (bs, 1H), 8.32-8.37 (m, 1H), 8.26 (s, 1H), 7.51-7.59 (m, 2H), 6.80 (bs, 2H), 6.53 (s, 1H), 5.99-6.04 (m, 1H), 5.10 (t, J=6.4 Hz, 2H), 5.00 (t, J=6.4 Hz, 2H), 1.31 (s, 9H), LCMS: 467.2[M+H].

### Example IA-24

### 1-(4-(4-amino-1-(tetrahydrofuran-3-yl)-1H-pyrazolo[3,4-d]pyrimidin-3-yl)-2-fluorophenyl)-3-(3-(tert-butyl)isoxazol-5-yl)urea

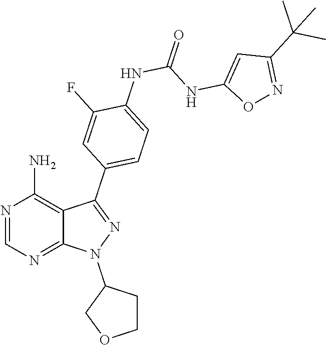 [SMILES: CC(C)(C)c1cc(NC(=O)Nc2ccc(-c3nn(C4CCOC4)c4ncnc(N)c34)cc2F)on1]

The title compound was prepared following the general procedure described for Example Ia-1, starting from 3-(4-amino-3-fluorophenyl)-1-(tetrahydrofuran-3-yl)-11H-pyrazolo[3,4-d]pyrimidin-4-amine (Ia-B11, 0.200 g, 0.59 mmol) and phenyl (3-(tert-butyl)isoxazol-5-yl)carbamate (Ia-C4, 0.154 g, 0.59 mmol), and was obtained as a white solid (0.030 g, 10% yield). H NMR (400 MHz, DMSO-d₆) δ=10.40 (bs, 1H), 8.92 (bs, 1H), 8.26-8.31 (m, 2H), 7.47-7.54 (m, 2H), 6.80 (bs, 2H), 6.11 (s, 1H), 5.47-5.53 (m, 1H), 4.05-4.13 (m, 2H), 3.87-3.96 (m, 2H), 2.40-2.41 (m, 2H), 1.27 (s, 9H), LCMS: 481.2[M+H].

### Example IA-25

### 1-(4-(4-amino-1-cyclopropyl-1H-pyrazolo[3,4-d]pyrimidin-3-yl)-2-fluorophenyl)-3-(5-(1-(trifluoromethyl)cyclopropyl)isoxazol-3-yl)urea

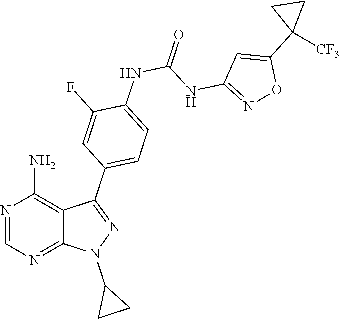 [SMILES: Nc1ncnc2c1c(-c1ccc(NC(=O)Nc3cc(C4(C(F)(F)F)CC4)on3)c(F)c1)nn2C1CC1]

The title compound was prepared following the general procedure described for Example Ia-1, starting from 3-(4-amino-3-fluorophenyl)-1-cyclopropyl-1H-pyrazolo[3,4-d]pyrimidin-4-amine (Ia-B1, 0.070 g, 0.24 mmol) and phenyl (5-(1-(trifluoromethyl)cyclopropyl)isoxazol-3-yl)carbamate (Ia-C7, 0.077 g, 0.24 mmol), and was obtained as an off-white solid; (0.013 mg, 11% yield). ¹H NMR (400 MHz, DMSO-d₆) δ=11.78 (bs, 1H), 8.76 (s, 1H), 7.43-7.45 (m, 2H), 6.84-6.88 (m, 2H), 5.46 (s, 2H), 3.91-3.97 (m, 1H), 1.49-1.54 (m, 4H), 1.19-1.22 (m, 2H), 1.10-1.15 (m, 2H); LCMS: 503.1[M+H].

### Example IA-26

### 1-(4-(4-amino-1H-pyrazolo[3,4-d]pyridin-3-yl)-2-fluorophenyl)-3-(5-(tert-butyl)isoxazol-3-yl)urea

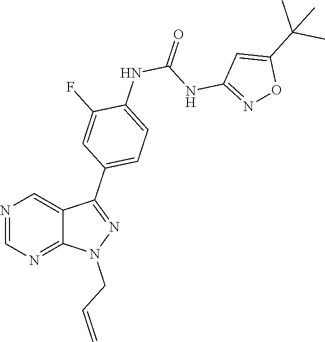 [SMILES: C=CCn1nc(-c2ccc(NC(=O)Nc3cc(C(C)(C)C)on3)c(F)c2)c2cncnc21]

The title compound was prepared following the general procedure described for Example Ia-1, starting from 1-allyl-3-(4-amino-3-fluorophenyl)-1H-pyrazolo[3,4-d]pyrimidin-4-amine (Ia-B6, 0.130 g, 0.45 mmol) and phenyl (5-(tert-butyl)isoxazol-3-yl)carbamate (Ia-C5, 0.119 g, 0.45 mmol), and was obtained as an off-white solid; (0.012 g, 6% yield). ¹H NMR (400 MHz, DMSO-d₆) δ=10.46 (bs, 1H), 9.52 (bs, 1H), 8.24-8.28 (m, 2H), 7.45-7.50 (m, 2H), 6.95 (bs, 2H), 6.52 (s, 1H), 6.00-6.10 (m, 1H), 5.18-5.21 (m, 2H), 5.13 (d, J=1.6 Hz, 2H), 1.31 (s, 9H); LCMS: 451.2[M+H].

### Example IA-27

### 1-(4-(4-amino-1-cyclopropyl-1H-pyrazolo[3,4-d]pyrimidin-3-yl)-2-fluorophenyl)-3-(3-(2-fluoropropan-2-yl)isoxazol-5-yl)urea

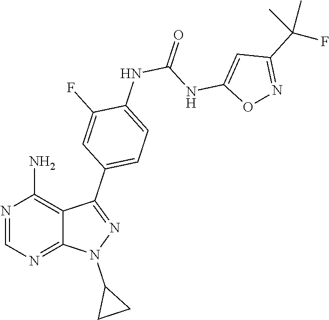 [SMILES: CC(C)(F)c1cc(NC(=O)Nc2ccc(-c3nn(C4CC4)c4ncnc(N)c34)cc2F)on1]

The title compound was prepared following the general procedure described for Example Ia-1, starting from 3-(4-amino-3-fluorophenyl)-1-cyclopropyl-1H-pyrazolo[3,4-d]pyrimidin-4-amine (Ia-B1, 0.050 g, 0.176 mmol) and phenyl (3-(2-fluoropropan-2-yl)isoxazol-5-yl)carbamate (Ia-C9, 0.047 g, 0.176 mmol), and was obtained as an off-white solid (0.007 mg, 9% yield). ¹H NMR (400 MHz, DMSO-d₆) δ8.32-8.35 (m, 1H), 8.25 (s, 1H), 7.32-7.37 (m, 2H), 5.92 (s, 1H), 3.83-3.89 (m, 1H), 1.66 (s, 3H), 1.61 (s, 3H), 1.20-1.24 (m, 2H), 1.07-1.09 (m, 2H); LCMS: 455.1[M+H].

### Example IA-28

### 1-(4-(4-amino-1-methyl-1H-pyrazolo[3,4-d]pyrimidin-3-yl)-2-chlorophenyl)-3-(3-(tert-butyl)isoxazol-5-yl)urea

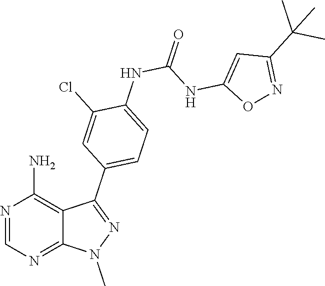 [SMILES: Cn1nc(-c2ccc(NC(=O)Nc3cc(C(C)(C)C)no3)c(Cl)c2)c2c(N)ncnc21]

The title compound was prepared following the general procedure described for Example Ia-1, starting from 3-(4-amino-3-chlorophenyl)-1-methyl-1H-pyrazolo[3,4-d]pyrimidin-4-amine (Ia-B22, 0.068 g, 0.248 mmol and phenyl (3-(tert-butyl)isoxazol-5-yl)carbamate (Ia-C4, 0.064 g, 0.248 mmol), and was obtained as an off-white solid; (0.005 g, 4% yield). ¹H NMR (400 MHz, CD₃OD) δ=8.70-8.68 (m, 1H), 7.47-7.67 (m, 2H), 7.00-7.02 (m, 1H), 6.20 (s, 1H), 4.10 (s, 3H), 1.34 (s, 9H), LCMS: 441.1[M+H].

### Example IA-29

### 1-(4-(4-amino-1-methyl-1H-pyrazolo[3,4-d]pyrimidin-3-yl)-2-fluorophenyl)-3-(3-(tert-butyl)isoxazol-5-yl)urea

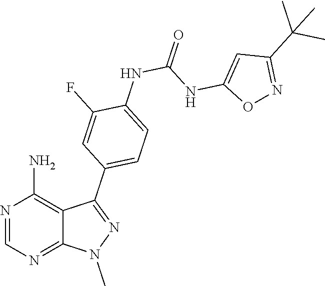 [SMILES: Cn1nc(-c2ccc(NC(=O)Nc3cc(C(C)(C)C)no3)c(F)c2)c2c(N)ncnc21]

The title compound was prepared following the general procedure described for Example Ia-1, starting from 3-(4-amino-3-fluorophenyl)-1-methyl-1H-pyrazolo[3,4-d]pyrimidin-4-amine (Ia-B23, 0.052 g, 0.201 mmol) and phenyl (3-(tert-butyl)isoxazol-5-yl)carbamate (Ia-C4, 0.052 g, 0.201 mmol), and was obtained as an off-white solid; (0.005 g, 6% yield). ¹H NMR (400 MHz, CD₃OD) δ=10.60 (bs, 1H), 9.05 (bs, 1H), 8.25-8.30 (m, 2H), 7.45-7.51 (m, 2H), 6.90 (s, 2H), 6.09 (s, 1H), 3.95 (s, 3H), 1.27 (s, 9H); LCMS: 423.1[M−H].

### Example IA-30

### 1-(4-(4-amino-1-cyclopropyl-1H-pyrazolo[4,3-c]pyridin-3-yl)-2-fluorophenyl)-3-(3-(tert-butyl)isoxazol-5-yl)urea

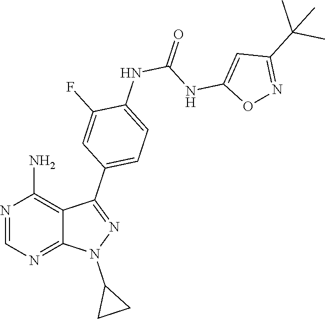 [SMILES: CC(C)(C)c1cc(NC(=O)Nc2ccc(-c3nn(C4CC4)c4ncnc(N)c34)cc2F)on1]

The title compound was prepared following the general procedure described for Example Ia-1, starting from 3-(4-amino-3-fluorophenyl)-1-cyclopropyl-1H-pyrazolo[4,3-c]pyridin-4-amine (Ia-B24, 0.070 g, 0.24 mmol) & phenyl (3-(tert-butyl)isoxazol-5-yl)carbamate (Ia-C4, 0.064 g, 0.24 mmol), and was obtained as an off-white solid (0.004 g, 4% yield). ¹H NMR (400 MHz, DMSO-d₆) δ=10.52 (bs, 1H), 9.09 (bs, 1H), 8.23-8.27 (m, 1H), 7.80 (s, 1H), 7.44-7.53 (m, 2H), 6.90 (d, J=6.0 Hz, 1H), 6.10 (s, 1H), 5.86 (bs, 2H), 3.72-3.74 (m, 1H), 1.27 (s, 9H), 1.12-1.13 (m, 4H), LCMS: 450.2[M+H].

### Example IA-31

### 1-(4-(4-amino-1-cyclopropyl-1H-pyrazolo[4,3-c]pyridin-3-yl)phenyl)-3-(3-(tert-butyl)isoxazol-5-yl)urea

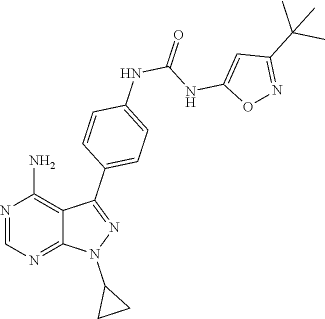 [SMILES: CC(C)(C)c1cc(NC(=O)Nc2ccc(-c3nn(C4CC4)c4ncnc(N)c34)cc2)on1]

The title compound was prepared following the general procedure described for Example Ia-1, starting from 3-(4-aminophenyl)-1-cyclopropyl-1H-pyrazolo[4,3-c]pyridin-4-amine (Ia-B25, 0.052 g, 0.2 mmol) and phenyl (3-(tert-butyl)isoxazol-5-yl)carbamate (Ia-C4, 0.05 g, 0.2 mmol), and was obtained as a white solid (0.007 g, 8% yield). ¹H NMR (400 MHz, DMSO-di) δ=10.85 (bs, 1H), 9.68 (bs, 1H), 7.78 (bs, 1H), 7.66 (d, J=8.4 Hz, 2H), 7.56 (d, J=8.4 Hz, 2H), 6.90 (d, J=6.0 Hz, 1H), 6.08 (s, 1H), 5.75 (bs, 2H), 3.68-3.73 (m, 1H), 1.26 (s, 9H), 1.11-1.12 (m, 4H); LCMS: 432.3[M+H].

### Intermediate IB-A1

### 4-chloro-1-((2-(trimethylsilyl)ethoxy)methyl)-1H-pyrrolo[2,3-b]pyridine

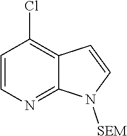 [SMILES: C[Si](C)(C)CCOCn1ccc2c(Cl)ccnc21]

### Method Ib-A:

NaH (60% dispersion in mineral oil, 0.118 g, 4.92 mmol) was added in portions to a stirred solution of 4-chloro-1H-pyrrolo[2,3-b]pyridine (0.500 g, 3.28 mmol) in DMF (5 mL) at 0° C. and the resulting suspension was stirred at 0° C. for 20 min. SEM-Cl (0.697 mL, 3.93 mmol) was then added and the reaction mixture was allowed to warm to 25° C. and was stirred for 5 h. Following completion of the reaction (as indicated by TLC), the mixture was quenched with ice-cold water (25 mL) and extracted with EtOAc (2×25 mL). The combined organic phases were dried over Na₂SO₄, filtered, and concentrated under reduced pressure to give crude material which was purified by Isolera (silica gel 230-400 mesh, eluting with 4% EtOAc in petroleum ether), affording the title compound as a yellow liquid (0.77 g, 82% yield). ¹H NMR (400 MHz, DMSO-d₆) δ=8.36 (d, J=6.8 Hz, 1H), 7.89 (d, J=4.8 Hz, 1H), 7.40 (s, 1H), 6.72 (d, J=4.8 Hz, 1H), 5.75 (s, 2H), 3.62 (t, J=10.8 Hz, 2H), 0.92 (t, J=10.4 Hz, 2H), −0.011 (s, 9H). LC-MS: 283.0 [M+H].

### Method Ib-B.

DIPEA (11.45 mL, 65.5 mmol) and SEM-Cl (8.14 mL, 45.9 mmol) were added to a stirred solution of 4-chloro-1H-pyrrolo[2,3-b]pyridine (5.00 g, 32.8 mmol) in acetonitrile (25 mL) at 0° C. and the resulting mixture was allowed to warm to 25° C. and was stirred for 12 h. Following completion of the reaction (as indicated by UPLC and TLC), the reaction mixture was concentrated under reduced pressure to yield crude material which was purified by Isolera (silica gel 230-400 mesh, eluting with 4% EtOAc in petroleum ether), affording the title compound as a yellow liquid (9.24 g, 99% yield). ¹H NMR (400 MHz, DMSO-d₆) δ=836 (d, J=6.8 Hz, 1H), 7.89 (d, J=4.8 Hz, 1H), 7.40 (s, 1H), 6.72 (d, J=4.8 Hz, 1H), 5.75 (s, 2H), 3.62 (t, J=10.8 Hz, 2H), 0.92 (t, J=10.4 Hz, 2H), −0.011 (s, 9H). LC-MS: 283.0 [M+H].

### Intermediate IB-A2

### 4-bromo-1-((2-(trimethylsilyl)ethoxy)methyl)-1H-pyrrolo[2,3-b]pyridine

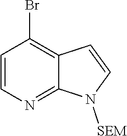 [SMILES: C[Si](C)(C)CCOCn1ccc2c(Br)ccnc21]

### Method Ib-A:

NaH (60% dispersion in mineral oil, 0.609 g, 15.23 mmol) was added in portions to a stirred solution of 4-bromo-1H-pyrrolo[2,3-b]pyridine (2.000 g, 10.15 mmol) in DMF (20 mL) at 0° C. and the resulting suspension was stirred at 0° C. for 20 min. SEM-Cl (2.031 g, 12.18 mmol) was then added and the reaction mixture was allowed to warm to 25° C. and was stirred for 3 h. Following completion of the reaction (as indicated by TLC), the mixture was quenched with ice-cold water (25 mL) and extracted with EtOAc (2×25 mL). The combined organic phases were dried over Na₂SO₄, filtered, and concentrated under reduced pressure to give crude material which was purified by Isolera (silica gel 230-400 mesh, eluting with 4% EtOAc in petroleum ether), affording the title compound as a colorless liquid (2.640 g, 79% yield). ¹H NMR (400 MHz, DMSO-d₆) δ=8.16 (d, J=6.8 Hz, 1H), 7.80 (d, J=4.80 Hz, 1H), 7.43-7.44 (m, 1H), 6.52-6.53 (m, 1H), 5.64 (bs, 2H), 3.51 (t, J=10.8 Hz, 2H), 0.81 (t, J=10.4 Hz, 2H), −0.11 (s, 9H) LC-MS: 327.0 [M+H].

### Method Ib-B:

DIPEA (0.886 mL, 5.08 mmol) and SEM-Cl (0.630 mL, 3.55 mmol) were added to a stirred solution of 4-bromo-1H-pyrrolo[2,3-b]pyridine (0.500 g, 2.54 mmol) in acetonitrile (10 mL) at 0° C. and the resulting mixture was allowed to warm to 25° C. and was stirred for 12 h. Following completion of the reaction (as indicated by UPLC and TLC), the reaction mixture was concentrated under reduced pressure to yield crude material which was purified by Isolera (silica gel 230-400 mesh, eluting with 4% EtOAc in petroleum ether), affording the title compound as a colorless liquid (0.680 g, 81% yield). ¹H NMR (400 MHz, DMSO-d₆) δ=8.16 (d, J=6.8 Hz, 1H), 7.80 (d, J=4.8 Hz, 1H), 7.43-7.44 (m, 1H), 6.52-6.53 (m, 1H), 5.64 (bs, 2H), 3.51 (t, J=10.8 Hz, 2H), 0.81 (t, J=10.4 Hz, 2H), −0.11 (s, 9H). LC-MS: 327.0 [M+H].

### Intermediate IB-A3

### 4-chloro-3-methyl-1-((2-(trimethylsilyl)ethoxy)methyl)-1H-pyrrolo[2,3-b]pyridine

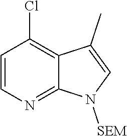 [SMILES: Cc1cn(COCC[Si](C)(C)C)c2nccc(Cl)c12]

The title compound was obtained following a similar procedure described for Intermediate Ib-A1 (Method Ib-A), starting from 4-chloro-3-methyl-1H-pyrrolo[2,3-b]pyridine (0.20 g, 0.60 mmol), NaH (60%, 0.04 g, 0.90 mmol) and SEM-Cl (0.24 g, 0.72 mmol), and was obtained as a colorless liquid (0.23 g, 90% yield). ¹H NMR (400 MHz, DMSO-d₆) δ=8.18 (d, J=5.2 Hz, 1H), 7.50 (s, 1H), 7.19 (d, J=5.2 Hz, 1H), 5.57 (s, 2H), 3.48 (t, J=8.0 Hz, 2H), 2.45 (s, 3H), 0.82 (t, J=8.0 Hz, 2H), 0.01 (s, 9H). LC-MS: 297.1 [M+H].

### Intermediate IB-B1

### tert-butyl (2-fluoro-4-hydroxyphenyl)carbamate

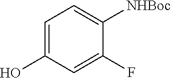 [SMILES: CC(C)(C)OC(=O)Nc1ccc(O)cc1F]

di-Tert-butyl dicarbonate (5.67 g, 26.0 mmol) was added to 4-amino-3-fluorophenol (3.00 g, 23.6 mmol) and indium (111) chloride (0.052 g, 0.236 mmol) in DCM (30 mL) and the resulting mixture was stirred at 40° C. for 3 h. Following completion of the reaction (as indicated by TLC), the reaction mixture was quenched with water (30 mL) and extracted with EtOAc (2×10 mL). The combined organic phases were dried over Na₂SO₄, filtered, and concentrated under reduced pressure to give crude material which was purified by Isolera (eluting with 15% EtOAc in petroleum ether), affording the title compound as a brown solid (4.93 g, 91% yield). ¹H NMR (400 MHz, DMSO-d₆) δ=9.70 (bs, 1H), 8.52 (bs, 1H), 7.13-7.19 (m, 1H), 6.51-6.58 (m, 2H), 1.42 (s, 9H). LC-MS: 226.1 [M−H].

### Intermediate IB-B2

### tert-butyl (3-fluoro-4-hydroxyphenyl)carbamate

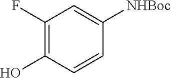 [SMILES: CC(C)(C)OC(=O)Nc1ccc(O)c(F)c1]

The title compound was obtained following a similar procedure described for Intermediate Ib-B1, starting from 4-amino-2-fluorophenol (0.300 g, 2.36 mmol), indium (III) chloride (0.0052 g, 0.0236 mmol), and di-tert-butyl dicarbonate (0.567 g, 2.60 mmol), and was obtained as a brown solid (0.390 g, 73% yield). ¹H NMR (400 MHz, DMSO-d₆) δ=9.39 (s, 1H), 9.21 (bs, 1H), 7.27-7.31 (m, 1H), 6.97-7.00 (m, 1H), 6.78-6.85 (m, 1H), 1.46 (s, 9H). LC-MS: 126.1 [M-Boc].

### Intermediate IB-B3

### tert-butyl (2,6-difluoro-4-hydroxyphenyl)carbamate

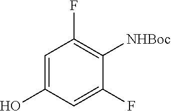 [SMILES: CC(C)(C)OC(=O)Nc1c(F)cc(O)cc1F]

The title compound was obtained following a similar procedure described for Intermediate Ib-B1, starting from 4-amino-3,5-difluorophenol (0.25 g, 1.722 mmol), indium (III) chloride (0.0038 mg, 0.017 mmol) and di-tert-butyl dicarbonate (0.41 g, 1.895 mmol), and was obtained as a brown solid (0.32 g, 76% yield). ¹H NMR (400 MHz, DMSO-d₆) δ=10.28 (s, 1H), 8.38 (bs, 1H), 6.45-6.50 (m, 2H), 1.42 (s, 9H). LC-MS: 244.1 [M+H].

### Intermediate IB-C1

### tert-butyl (2-fluoro-4-((1-((2-(trimethylsilyl)ethoxy)methyl)-1H-pyrrolo[2,3-b]pyridin-4-yl)oxy)phenyl)carbamate

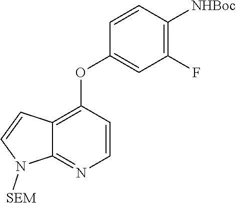 [SMILES: CC(C)(C)OC(=O)Nc1ccc(Oc2ccnc3c2ccn3COCC[Si](C)(C)C)cc1F]

### Method Ib-A:

K₂CO₃ (2.452 g, 17.750 mmol) and XPhos (0.385 g, 0.807 mmol) were added to a solution of 4-bromo-1-((2-(trimethylsilyl)ethoxy)methyl)-1H-pyrrolo[2,3-b]pyridine (Ib-A2, 2.640 g, 8.070 mmol) and tert-butyl (2-fluoro-4-hydroxyphenyl)carbamate (Ib-B1, 1.943 g, 8.550 mmol) in toluene (40 mL) and the resulting suspension was purged with N₂ for 10 min. Pd₂(dba)₃ (0.369 g, 0.403 mmol) was then added and the reaction mixture was stirred at 100° C. for 12 h. Following completion of the reaction (as indicated by TLC), the reaction mixture was filtered through a pad of diatomaceous earth which was then rinsed with EtOAc (2×15 mL). The combined filtrates were concentrated under reduced pressure to yield crude material which was purified by Isolera (eluting with 20% EtOAc in petroleum ether), affording the title compound as a brown gum (2.70 g, 70% yield). ¹H NMR (400 MHz, DMSO-d₆) δ=9.70 (bs, 1H), 8.16-8.18 (m, 1H), 7.55-7.64 (m, 2H), 7.15-7.20 (m, 1H), 6.96-6.99 (m, 1H), 6.53-6.56 (m, 1H), 6.29-6.31 (m, 1H), 5.61 (s, 2H), 3.51 (t, J=10.4 Hz, 2H), 1.46 (s, 9H), 0.81 (t, J=10.8 Hz, 2H), 0.11 (s, 9H). LC-MS: 474.2 [M−H].

### Method Ib-B:

K₂CO₃ (1.075 g, 7.780 mmol) and XPhos (0.169 g, 0.354 mmol) were added to a solution of 4-chloro-1-((2-(trimethylsilyl)ethoxy)methyl)-1H-pyrrolo[2,3-b]pyridine (Ib-A1, 1.000 g, 3.540 mmol) and tert-butyl (2-fluoro-4-hydroxyphenyl)carbamate (Ib-B1, 0.852 g, 3.75 mmol) in toluene (20 mL) and the resulting suspension was purged with N₂ for 10 min. Pd₂(dba)₃ (0.162 g, 0.177 mmol) was then added and the reaction mixture was stirred at 100° C. for 12 h. Following completion of the reaction (as indicated by TLC), the reaction mixture was filtered through a pad of diatomaceous earth which was then rinsed with EtOAc (10 mL×2). The combined filtrates were concentrated under reduced pressure to yield crude material which was purified by Isolera (eluting with 20% EtOAc in petroleum ether), affording the title compound as a brown gum (1.30 g, 80% yield). ¹H NMR (400 MHz, DMSO-d₆) J=9.70 (bs, 1H), 8.16-8.18 (m, 1H), 7.55-7.64 (m, 2H), 7.15-7.20 (m, 1H), 6.96-6.99 (m, 1H), 6.53-6.56 (m, 1H), 6.29-6.31 (m, 1H), 5.61 (s, 2H), 3.51 (t, J=10.4 Hz, 2H), 1.46 (s, 9H), 0.81 (t, J=10.8 Hz, 2H), 0.11 (s, 9H). LC-MS: 474.2 [M−H].

### Intermediate IB-C2

### tert-butyl (4-((3-chloro-1-((2-(trimethylsilyl)ethoxy)methyl)-1H-pyrrolo[2,3-H]pyridin-4-yl)oxy)-2-fluorophenyl)carbamate

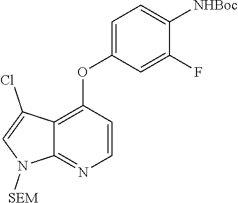 [SMILES: CC(C)(C)OC(=O)Nc1ccc(Oc2ccnc3c2c(Cl)cn3COCC[Si](C)(C)C)cc1F]

NCS (0.792 g, 5.93 mmol) was added to a solution of tert-butyl (2-fluoro-4-((1-((2-(trimethylsilyl)ethoxy)methyl)-1H-pyrrolo[2,3-b]pyridin-4-yl)oxy)phenyl)carbamate (Ib-C1, 2.700 g, 5.70 mmol) in acetonitrile (30 mL) and the resulting mixture was stirred at 60° C. overnight. Following completion of the reaction (as indicated by UPLC), the reaction mixture was concentrated under reduced pressure to yield a residue which was taken in EtOAc (100 mL) and washed with brine (10 mL). The organic phase was dried over Na₂SO₄, filtered, and concentrated under reduced pressure to yield crude material which was purified by Isolera (eluting with 8% EtOAc in petroleum ether), affording the title compound as a colorless gum (2.20 g, 73% yield). ¹H NMR (400 MHz, DMSO-d₆) 3=9.04 (bs, 1H), 8.22 (d, J=5.6 Hz, 1H), 7.82 (s, 1H), 7.62-7.66 (m, 1H), 7.19-7.22 (m, 1H), 6.97-7.00 (m, 1H), 6.51 (d, J=5.6 Hz, 1H), 5.61 (s, 2H), 3.54 (t, J=8.0 Hz, 2H), 1.47 (s, 9H), 0.84 (t, J=7.6 Hz, 2H), −0.08 (s, 9H). LC-MS: 508.1 [M−H].

### Intermediate IB-C3

### tert-butyl (2-fluoro-4-((3-methyl-1-((2-(trimethylsilyl)ethoxy)methyl)-1H-pyrrolo[2,3-b]pyridin-4-yl)oxy)phenyl)carbamate

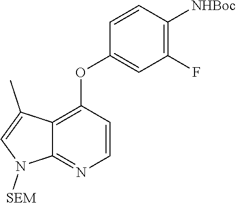 [SMILES: Cc1cn(COCC[Si](C)(C)C)c2nccc(Oc3ccc(NC(=O)OC(C)(C)C)c(F)c3)c12]

The title compound was obtained following a similar procedure described for Intermediate Ib-C1 (Method Ib-B), starting from 4-chloro-3-methyl-1-((2-(trimethylsilyl)ethoxy)methyl)-1H-pyrrolo[2,3-b]pyridine (Ib-A3, 0.23 g, 0.77 mmol), tert-butyl (2-fluoro-4-hydroxyphenyl)carbamate (Ib-B1, 0.187 g, 0.82 mmol), K₂CO₃ (0.037 g, 0.07 mmol), XPhos (0.234 g, 1.69 mmol) and Pd₂(dba)₃ (0.035 g, 0.04 mmol) as brown gum (0.29 g, 76% yield). ¹H NMR (400 MHz, DMSO-d₆) δ=8.99 (bs, 1H), 8.11 (d, J=5.20 Hz, 1H), 7.32 (s, 1H), 7.11-7.15 (m, 1H), 6.94 (d, J=8.80 Hz, 1H), 6.52-6.57 (m, 1H), 6.43 (d, J=5.60 Hz, 1H), 5.55 (s, 2H), 3.50 (t, J=8.00 Hz, 2H), 2.32 (s, 3H), 1.47 (s, 9H), 0.83 (t, =8.40 Hz, 2H), −0.08 (s, 9H). LC-MS: 488.1 [M+H].

### Intermediate IB-C4

### tert-butyl (4-((3-cyclopropyl-1-((2-(trimethylsilyl)ethoxy)methyl)-1H-pyrrolo[2,3-b]pyridin-4-yl)oxy)-2-fluorophenyl)carbamate

### Step 1: Synthesis of tert-butyl (4-((3-chloro-1-((2-(trimethylsilyl)ethoxy)methyl)-1H-pyrrolo[2,3-b]pyridin-4-yl)oxy)-2-fluorophenyl)carbamate

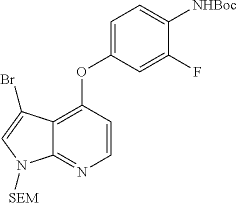 [SMILES: CC(C)(C)OC(=O)Nc1ccc(Oc2ccnc3c2c(Br)cn3COCC[Si](C)(C)C)cc1F]

NBS (0.217 g, 1.217 mmol) was added to a solution of tert-butyl (2-fluoro-4-((1-((2-(trimethylsilyl)ethoxy)methyl)-1H-pyrrolo[2,3-b]pyridin-4-yl)oxy)phenyl)carbamate (Ib-C1, 0.524 g, 1.106 mmol) in DMF (7 mL) and the resulting mixture was stirred at 25° C. overnight. Following completion of the reaction (as indicated by UPLC), the reaction mixture was poured into crushed ice (70 g), stirred at 25° C. for 15 minutes, and extracted with EtOAc (3×50 mL). The combined organic phases were washed with brine (2×10 mL), dried over Na₂SO₄ filtered, and concentrated under reduced pressure to give crude material which was purified by flash chromatography (silica gel 230-400 mesh, eluting with 30% EtOAc in petroleum ether), affording the title compound as an off-white solid (0.514 g, 59% yield). ¹NMR (400 MHz, DMSO-d₆) δ=903 (bs, 1H), 8.17-8.22 (m, 1H), 7.85 (s, 1H), 7.57-7.66 (m, 1H), 7.16-7.21 (m, 1H), 6.96-6.99 (m, 1H), 6.51-6.57 (m, 1H), 5.61 (s, 2H), 3.51 (t, J=6.8 Hz, 2H), 1.47 (s, 9H), 0.86 (t, J=4.8 Hz, 2H), 0.00 (s, 9H). LC-MS: 552.2 (M+H).

### Step 2: Synthesis of tert-butyl (4-((3-cyclopropyl-1-((2-(trimethylsilyl)ethoxy)methyl)-1H-pyrrolo[2,3-b]pyridin-4-yl)ethoxy)-2-fluorophenyl)carbamate

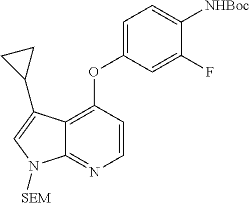 [SMILES: CC(C)(C)OC(=O)Nc1ccc(Oc2ccnc3c2c(C2CC2)cn3COCC[Si](C)(C)C)cc1F]

Potassium cyclopropyl trifluoroborane (0.074 g, 0.498 mmol), tricyclohexylphosphine (0.005 g, 0.018 mmol), palladium (II) acetate (0.002 g, 0.009 mmol), and K₂CO₃ (0.125 g, 0.905 mmol) were added to a solution of tert-butyl (4-((3-bromo-1-((2-(trimethylsilyl)ethoxy)methyl)-1H-pyrrolo[2,3-b]pyridin-4-yl)oxy)-2-fluorophenyl)carbamate (0.250 g, 0.452 mmol) in toluene (3 mL) and water (1 mL). The resulting suspension was degassed for 15 minutes then stirred at 90° C. in a sealed tube overnight. Following completion of the reaction (as indicated by UPLC and TLC), the reaction mixture was filtered through a pad of diatomaceous earth which was then rinsed with EtOAc. The combined filtrates were concentrated under reduced pressure to give crude material which was purified by RP-HPLC (using 0.1% TFA in water. ACN) to afford the title compound as a colorless gum (0.070 g, 27.9% Yield). LC-MS: 514.2 (M+H).

### Intermediate IB-C5

### tert-butyl (3-fluoro-4-((1-((2-(trimethylsilyl)ethoxy)methyl)-1H-pyrrolo[2,3-b]pyridin-4-yl)oxy)phenyl)carbamate

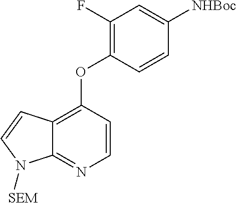 [SMILES: CC(C)(C)OC(=O)Nc1ccc(Oc2ccnc3c2ccn3COCC[Si](C)(C)C)c(F)c1]

The title compound was obtained following a similar procedure described for Intermediate Ib-C1 (Method Ib-A), starting from 4-bromo-1-((2-(trimethylsilyl)ethoxy)methyl)-1H-pyrrolo[2,3-b]pyridine (Ib-A2, 0.200 g, 0.61 mmol), tert-butyl (3-fluoro-4-hydroxyphenyl)carbamate (Ib-B2, 0.147 g, 0.64 mmol), K₂CO₃ (0.195 g, 1.40 mmol), XPhos (0.031 g, 0.06 mmol), and Pd₂(dba)₃ (0.028 g, 0.03 mmol), and was obtained as a yellow gum (0.075 g, 26% yield). LC-MS: 474.2 [M+H].

### Intermediate IB-C6

### tert-butyl (4-((3-chloro-1-((2-(trimethylsilyl)ethoxy)methyl)-1H-pyrrolo[2,3-b]pyridin-4-yl)oxy)-3-fluorophenyl)carbamate

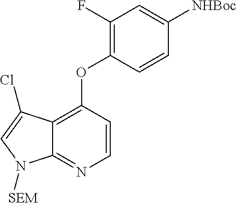 [SMILES: CC(C)(C)OC(=O)Nc1ccc(Oc2ccnc3c2c(Cl)cn3COCC[Si](C)(C)C)c(F)c1]

The title compound was obtained following a similar procedure described for Intermediate Ib-C₂, starting from tert-butyl (3-fluoro-4-((1-((2-(trimethylsilyl)ethoxy)methyl)-1H-pyrrolo[2,3-b]pyridin-4-yl)oxy)phenyl)carbamate (Ib-C5, 0.075 g, 0.15 mmol) and NCS (0.022 g, 0.16 mmol), and was obtained as a yellow gum (0.10 g, 37% yield). LC-MS: 508.1 [M−H].

### Intermediate IB-C7

### tert-butyl (2,6-difluoro-4-((1-((2-(trimethylsilyl)ethoxy)methyl)-1H-pyrrolo[2,3-b]pyridin-4-yl)oxy)phenyl)carbamate

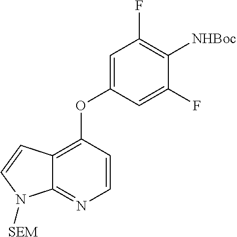 [SMILES: CC(C)(C)OC(=O)Nc1c(F)cc(Oc2ccnc3c2ccn3COCC[Si](C)(C)C)cc1F]

The title compound was obtained following a similar procedure described for Intermediate Ib-C1 (Method Ib-A), starting from 4-bromo-1-((2-(trimethylsilyl)ethoxy)methyl)-1H-pyrrolo[2,3-b]pyridine (Ib-A2, 0.100 g, 0.30 mmol), tert-butyl (2,6-difluoro-4-hydroxyphenyl)carbamate (Ib-B3, 0.079 g, 0.32 mmol), K₂CO₃ (0.091 g, 0.66 mmol), XPhos (0.014 g, 0.03 mmol), and Pd₂(dba)₃ (0.014 g, 0.015 mmol), and was obtained as a yellow gum (0.09 g, 60% yield). ¹H NMR (400 MHz, DMSO-d₆) δ=8.80 (bs, 1H), 8.24 (d, J=5.6 Hz, 1H), 7.61 (d, J=3.6 Hz, 1H), 7.07 (d, J=8.4 Hz, 2H), 6.71 (d, J=5.6 Hz, 1H), 6.32 (s, 1H), 5.64 (s, 2H), 3.53 (t, J==8.0 Hz, 2H), 1.44 (s, 9H), 0.82 (t, J=7.6 Hz, 2H), −0.11 (s, 9H). LC-MS: 492.2 [M+H].

### Intermediate IB-C8

### tert-butyl (4-((3-chloro-1-((2-(trimethylsilyl)ethoxy)methyl)-1H-pyrrolo[2,3-b]pyridin-4-yl)oxy)-2,6-difluorophenyl)carbamate

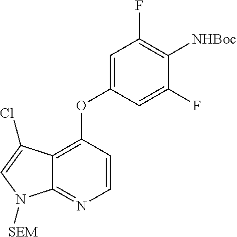 [SMILES: CC(C)(C)OC(=O)Nc1c(F)cc(Oc2ccnc3c2c(Cl)cn3COCC[Si](C)(C)C)cc1F]

The title compound was obtained following a similar procedure described for Intermediate Ib-C2, starting from tert-butyl (2,6-difluoro-4-((1-((2-(trimethylsilyl)ethoxy)methyl)-1H-pyrrolo[2,3-b]pyridin-4-yl)oxy)phenyl)carbamate (Ib-C7, 0.100 g, 0.20 mmol) and NCS (0.028 g, 0.21 mmol), and was obtained as a brown gum (0.16 g, quantitative yield). LC-MS: 526.2 [M−H].

### Intermediate IB-C9

### tert-butyl (4-((1-((2-(trimethylsilyl)ethoxy)methyl)-1H-pyrrolo[2,3-b]pyridin-4-yl)oxy)cyclohexyl)carbamate

### Step 1: Synthesis 4-((tert-butoxycarbonyl)amino)cyclohexyl 4-methylbenzenesulfonate

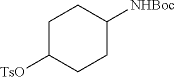 [SMILES: Cc1ccc(S(=O)(=O)OC2CCC(NC(=O)OC(C)(C)C)CC2)cc1]

DMAP (0.057 g, 0.464 mmol) and 4-toluenesulfonyl chloride (1.018 g, 5.340 mmol) were added to tert-butyl (4-hydroxycyclohexyl)carbamate (1.000 g, 4.640 mmol) in pyridine (15 ml) at 0° C. and the resulting solution was stirred at 25° C. for 5 days. Following completion of the reaction (as indicated by TLC), the reaction mixture was concentrated under reduced pressure to yield a residue which was diluted with water (30 mL) and extracted with EtOAc (2×30 mL). The combined organic phases were washed with 0.1 M HCl (10 mL), water (20 mL) and brine (20 mL), dried over Na₂SO₄, filtered, and concentrated under reduced pressure to give crude material which was purified by flash chromatography (silica gel 230-400 mesh, eluting with 15% EtOAc in petroleum ether), affording the title compound as a white solid (0.50 g, 29% yield). ¹H NMR (400 MHz, DMSO-d₆) δ=7.80 (d, J=10.8 Hz, 2H), 7.47 (d, 1=11.2 Hz, 2H), 6.71-6.74 (m, 1H), 4.30-4.38 (m, 1H), 3.16-3.23 (m, 1H), 2.42 (s, 3H), 1.70-1.78 (m, 4H), 1.41-1.56 (m, 2H), 1.39 (s, 9H), 1.11-1.24 (m, 2H). LC-MS: 270.0 [M-Boc].

### Step 2: Synthesis of tert-butyl (4-((1H-pyrrolo[2,3-b]pyridin-4-yl)oxy)cyclohexyl)carbamate

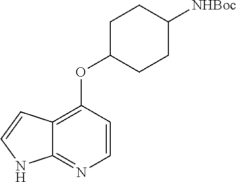 [SMILES: CC(C)(C)OC(=O)NC1CCC(Oc2ccnc3[nH]ccc23)CC1]

Cesium carbonate (0.364 g, 1.118 mmol) was added to a solution of 1H-pyrrolo[2,3-b]pyridin-4-ol (0.150 g, 1.118 mmol) and 4-((tert-butoxycarbonyl)amino)cyclohexyl 4-methylbenzenesulfonate (0.413 g, 1.118 mmol) in DMF (5 mL) and the resulting mixture was stirred at 90° C. for 16 h. Following completion of the reaction (as indicated by TLC), the reaction mixture was poured into crushed ice (50 g) and extracted with EtOAc (2×25 mL). The combined organic phases were washed with brine (20 mL), dried over Na₂SO₄, filtered, and concentrated under reduced pressure to give crude material which was purified by flash chromatography (silica gel 230-400 mesh, eluting with 20% EtOAc in petroleum ether), affording the title compound as a white solid (0.10 g, 25% yield). LC-MS: 270.0 [M-Boc].

### Step 3: tert-butyl (4-((1-((2-(trimethylsilyl)ethoxy)methyl)-1H-pyrrolo[2,3-b]pyridin-4-yl)oxy)cyclohexylcarbamate

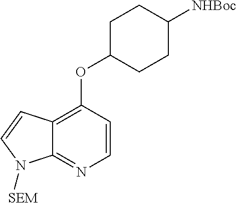 [SMILES: CC(C)(C)OC(=O)NC1CCC(Oc2ccnc3c2ccn3COCC[Si](C)(C)C)CC1]

DIPEA (0.042 ml, 0.241 mmol) and SEM-Cl (0.043 ml, 0.241 mmol) were added to a solution of tert-butyl (4-((1H-pyrrolo[2,3-b]pyridin-4-yl)oxy)cyclohexyl)carbamate (0.080 g, 0.241 mmol) in acetonitrile (5 mL) at 0° C. and the resulting mixture was stirred at 25° C. for 12 h. Following completion of the reaction (as indicated by UPLC and TLC), the reaction mixture was concentrated under reduced pressure to give crude material which was purified by Isolera (silica gel 230-400 mesh, eluting with 20% EtOAc in petroleum ether), affording the title compound as a brown solid (0.08 g, 62% yield). LC-MS: 462.2 [M+H].

### Intermediate IB-C10

### tert-butyl (4-((3-chloro-1-((2-(trimethylsilyl)ethoxy)methyl)-1H-pyrrolo[2,3-b]pyridin-4-yl)oxy)cyclohexyl)carbamate

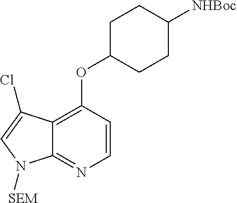 [SMILES: CC(C)(C)OC(=O)NC1CCC(Oc2ccnc3c2c(Cl)cn3COCC[Si](C)(C)C)CC1]

The title compound was obtained following a similar procedure described for step 3 of Intermediate Ib-D8 starting from (4-((1-((2-(trimethylsilyl)ethoxy)methyl)-1H-pyrrolo[2,3-b]pyridin-4-yl)oxy)cyclohexyl)carbamate (0.08 g, 0.173 mmol) and NCS (0.026 g), and was obtained as a yellow gum (0.08 g, 78% yield). LC-MS: 496.3 [M+H].

### Intermediate IB-C11

### tert-butyl (2-fluoro-4-((2-methyl-1-((2-(trimethylsilyl)ethoxy)methyl)-1H-pyrrolo[2,3-b]pyridin-4-yl)oxy)phenyl)carbamate

### Step 1: Synthesis of 4-chloro-1-(phenylsulfonyl-1H-pyrrolo[2,3-b]pyridine

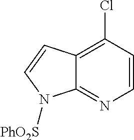

DMAP (0.080 g, 0.655 mmol) and benzenesulfonyl chloride (1.389 g, 7.860 mmol) were added to a solution of 4-chloro-1H-pyrrolo[2,3-b]pyridine (1.000 g, 6.550 mmol) in dry DCM (15 mL) at 0° C. and the resulting mixture was stirred at 25° C. for 15 h. Following completion of the reaction (as indicated by TLC), the reaction mixture was diluted with DCM (30 mL) then washed with 1 M HCl (20 mL), aqueous NaHCO₃(20 mL), and brine (20 mL). The organic layer was dried over Na₂SO₄, filtered, and concentrated under reduced pressure to give crude material which was purified by flash chromatography (silica gel 230-400 mesh, eluting with 5% EtOAc in petroleum ether), affording the title compound as a white solid (1.20 g, 62% yield). ¹H NMR (400 MHz, DMSO-d₆) δ=8.34 (d, J=5.2 Hz, 1H), 8.12-8.15 (m, 2H), 8.05 (d, J=4.0 Hz, 1H), 7.71-7.75 (m, 1H), 7.61-7.65 (m, 2H), 7.45 (d, J=5.2 Hz, 1H), 6.87 (d, J=4.0 Hz, 1H), LC-MS: 293.0 [M+H].

### Step 2: Synthesis of 4-chloro-2-methyl-1-(phenylsulfonyl)-1H-pyrrolo[2,3-b]pyridine

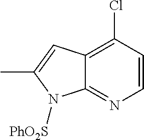

LDA (2M in THF, 3.42 ml, 6.83 mmol) was slowly to a solution of 4-chloro-1-(phenylsulfonyl)-1H-pyrrolo[2,3-b]pyridine (1.00 g, 3.42 mmol) in dry THF (15 mL) under N2 atmosphere at −78° C. in a 50 mL sealed tube. The resulting mixture was stirred at 78° C. for 1 h then Mel (6.38 ml, 102 mmol) was added and the solution was allowed to slowly warm to 25° C. and was stirred for another 16 h. Following completion of the reaction (as indicated by LC-MS), the reaction mixture was quenched with a saturated NH₄Cl solution (15 mL) and extracted with EtOAc (2.30 mL). The combined organic phases were dried over Na₂SO₄, filtered, and concentrated under reduced pressure to give the title compound (1.0 g, 57% yield) as a pale yellow gum which was used without further purification. ¹H NMR (400 MHz, DMSO-d₆) δ=8.25 (d, J=5.6 Hz, 1H), 8.11-8.13 (m, 2H), 7.70-7.74 (m, 1H), 7.60-7.64 (m, 2H), 7.40 (d, J=5.2 Hz, 1H), 6.67-6.67 (m, 1H), 2.50 (s, 3H). LC-MS: 307.0 [M+H].

### Step 3: Synthesis of 4-chloro-2-methyl-1H-pyrrolo[2,3-b]pyridine

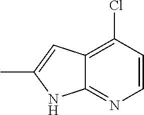

K₂CO₃ (2.253 g, 16.30 mmol) was added to a solution of 4-chloro-2-methyl-1-(phenylsulfonyl)-1H-pyrrolo[2,3-b]pyridine (1.000 g, 3.26 mmol) in MeOH (10 mL) and water (3.33 mL) and the resulting mixture was stirred at 60° C. for 16 h. Following completion of the reaction (as indicated by LC-MS), the mixture was cooled to 25° C. and the resulting solid was filtered, washed with water (2-5 mL), and dried to give the title compound as an off-white solid (0.50 g, 85% yield). ¹H NMR (400 MHz, DMSO-d₆) δ=11.86 (bs, 1H), 8.05 (d, J=6.8 Hz, 1H), 7.11 (d, J=6.8 Hz, 1H), 6.21 (d, J=1.21 Hz, 1H), 2.42 (s, 3H). LC-MS: 167.1 [M+H].

### Step 4: Synthesis of 4-chloro-2-methyl-1-((2-(trimethylsilyl)ethoxy)methyl)-1H-pyrrolo[2,3-b]pyridine

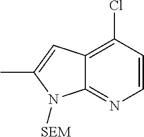 [SMILES: Cc1cc2c(Cl)ccnc2n1COCC[Si](C)(C)C]

The title compound was obtained following a similar procedure described for step 3 of Intermediate Ib-C9, starting from 4-chloro-2-methyl-1H-pyrrolo[2,3-b]pyridine (0.75 g, 4.50 mmol), DIPEA (1.455 mL, 8.10 mmol), and SEM-Cl (1.118 ml, 6.30 mmol), and was obtained as a pale yellow liquid (0.75 g, 53% yield). ¹H NMR (400 MHz, DMSO-d₆) δ=8.39 (d, =5.2 Hz, 1H), 7.63 (d, J=5.2 Hz, 1H), 6.99 (d, J=1.2 Hz, 1H), 4.67 (s, 2H), 3.54-3.59 (m, 2H), 2.59 (s, 3H), 0.85-0.89 (m, 2H), 0.00 (s, 9H). LC-MS: 267.1 [M+H].

### Step 5: Synthesis of tert-butyl (2-fluoro-4-((2-methyl-1-((2-(trimethylsilyl)ethoxy)methyl)-1H-pyrrolo[2,3-b]pyridin-4-yl)oxy)phenyl)carbamate

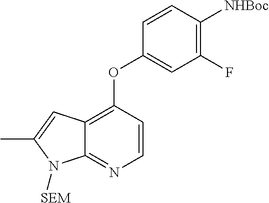 [SMILES: Cc1cc2c(Oc3ccc(NC(=O)OC(C)(C)C)c(F)c3)ccnc2n1COCC[Si](C)(C)C]

The title compound was obtained following a similar procedure described for Intermediate Ib-C1 (Method Ib-B), starting from 4-chloro-2-methyl-1-((2-(trimethylsilyl)ethoxy)methyl)-1H-pyrrolo[2,3-b]pyridine (0.700 g, 2.358 mmol), tert-butyl (2-fluoro-4-hydroxyphenyl)carbamate (Ib-B1, 0.643 g, 2.830 mmol)), K₂CO₃ (0.717 g, 5.190 mmol), XPhos (0.112 g, 0.236 mmol), and Pd₂(dba)₃ (0.108 g, 0.118 mmol), and was obtained as a brown gum (0.47 g, 38% yield). LC-MS: 488.2 [M+H].

### Intermediate IB-C12

### tert-butyl (4-((3-chloro-2-methyl-1-((2-(trimethylsilyl)ethoxy)methyl(yl)-1H-pyrrolo[2,3-b]pyridin-4-yl)oxy)-2-fluorophenyl)carbamate

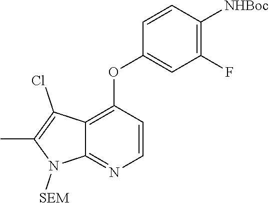 [SMILES: Cc1c(Cl)c2c(Oc3ccc(NC(=O)OC(C)(C)C)c(F)c3)ccnc2n1COCC[Si](C)(C)C]

The title compound was obtained following a similar procedure described for Intermediate Ib-C2, starting from tert-butyl (2-fluoro-4-((2-methyl-1-((2-(trimethylsilyl)ethoxy)methyl)-1H-pyrrolo[2,3-b]pyridin-4-yl)oxy)phenyl)carbamate (Ib-C11, 0.280 g, 0.460 mmol) and NCS (0.055 g, 0.413 mmol), and was obtained as a yellow gum (0.11 g, 33% yield). LC-MS: 522.2 [M−H].

### Intermediate IB-C13

### tert-butyl (2-fluoro-4-((7-((2-(trimethylsilyl)ethoxy)methyl)-7H-pyrrolo[2,3-d]pyrimidin-4-yl)oxy)phenyl)carbamate

### Step 1: Synthesis of 4-chloro-7-((2-(trimethylsilyl)ethoxy)methyl)-7H-pyrrolo[2,3-d]pyrimidine

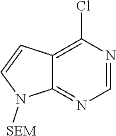 [SMILES: C[Si](C)(C)CCOCn1ccc2c(Cl)ncnc21]

The title compound was obtained following a similar procedure described for Intermediate Ib-A1 (Method Ib-A), starting from 4-chloro-7H-pyrrolo[2,3-d]pyrimidine (0.250 g, 1.62 mmol), NaI (60% in mineral oil, 0.098 g, 2.44 mmol), and SEM-Cl (0.34 mL, 1.95 mmol), and was obtained as a pale brown gum (0.22 g, 48% yield). ¹H NMR (400 MHz, DMSO-d₆) δ=8.69 (s, 1H), 7.89 (d, J=4.8 Hz, 1H), 6.73 (d, J=4.8 Hz, 1H), 5.65 (s, 2H), 3.52 (t, J=10.8 Hz, 2H), 0.82 (t, J=10.8 Hz, 2H), −0.10 (s, 9H). LC-MS: 284.0 [M+H].

### Step 2: Synthesis of tert-butyl (2-fluoro-4-((7-((2-(trimethylsilyl)ethoxy)methyl)-7H-pyrrolo[2,3-d]pyrimidin-4-yl)oxy)phenyl))carbamate

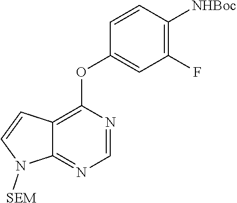 [SMILES: CC(C)(C)OC(=O)Nc1ccc(Oc2ncnc3c2ccn3COCC[Si](C)(C)C)cc1F]

The title compound was obtained following a similar procedure described for Intermediate Ib-C1 (Method Ib-B), starting from 4-chloro-7-((2-(trimethylsilyl)ethoxy)methyl)-7H-pyrrolo[2,3-d]pyrimidine (0.115 g, 0.40 mmol), tert-butyl (2-fluoro-4-hydroxyphenyl)carbamate (Ib-B1, 0.098 g 0.43 mmol), K₂CO₃ (0.124 g, 0.89 mmol), XPhos (0.020 g, 0.04 mmol), and Pd₂(dba)₃ (0.019 g, 0.02 mmol), and was obtained as a white solid (0.16 g, 84% yield). ¹H NMR (400 MHz, DMSO-d₆) δ=9.09 (bs, 1H), 8.48 (s, 1H), 7.75 (d, J=5.2 Hz, 1H), 7.65-7.71 (m, 1H), 7.34-7.39 (m, 1H), 7.14-7.17 (m, 1H), 6.71 (d, J=4.8 Hz, 1H), 5.70 (s, 2H), 3.61 (t, J=10.8 Hz, 2H), 1.55 (s, 9H), 0.92 (t, J=10.8 Hz, 2H), 0.00 (s, 9H) LC-MS: 475.2 [M+H].

### Intermediate IB-C14

### tert-butyl (4-((5-chloro-7-((2-(trimethylsilyl)ethoxy)methyl)-7H-pyrrolo[2,3-d]pyrimidin-4-yl)oxy)-2-fluorophenyl)carbamate

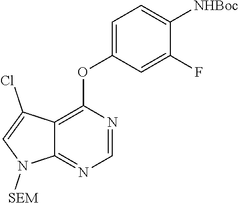 [SMILES: CC(C)(C)OC(=O)Nc1ccc(Oc2ncnc3c2c(Cl)cn3COCC[Si](C)(C)C)cc1F]

The title compound was obtained following a similar procedure described for Intermediate Ib-C2, starting from tert-butyl (2-fluoro-4-((7-((2-(trimethylsilyl)ethoxy)methyl)-7H-pyrrolo[2,3-d]pyrimidin-4-yl)oxy)phenyl)carbamate (Ib-C13, 0.25 g, 0.527 mmol) and NCS (0.07 g, 0.527 mmol), and was obtained as a colorless gum (0.10 g, 37% yield). LC-MS: 509.2 [M−H].

### Intermediate IB-D1

### 4-((3-chloro-1H-pyrrolo[2,3-b]pyridin-4-yl)oxy)-2-fluoroaniline

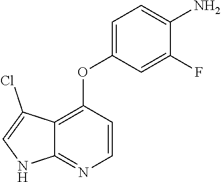 [SMILES: Nc1ccc(Oc2ccnc3[nH]cc(Cl)c23)cc1F]

TFA (10 mL, 4.33 mmol) was added to a solution of tert-butyl (4-((3-chloro-1-((2-(trimethylsilyl)ethoxy)methyl)-1H-pyrrolo[2,3-b]pyridin-4-yl)oxy)-2-fluorophenyl)carbamate (Ib-C2, 2.200 g, 4.33 mmol) in DCM (30 mL) at 0° C. and the resulting mixture was stirred at 25° C. for 12 h. Following completion of the reaction (as indicated by UPLC), the reaction mixture was concentrated under reduced pressure to give a crude residue. This brown gum was taken in MeOH (20 mL) and water (5 mL) then K₂CO₃ (1.795 g, 12.99 mmol) was added and the resulting mixture was stirred at 25° C. for 3 h. Following completion of the reaction (as indicated by UPLC), the reaction mixture was concentrated under reduced pressure to give crude material which was taken in DCM (100 mL) then washed with water (25 mL) and brine (25 mL). The organic layer was dried over Na₂SO₄, filtered, and concentrated under reduced pressure to give crude product which was purified flash chromatography (silica gel 230-400 mesh, eluting with 30% EtOAc in petroleum ether), affording the title compound as a pale brown solid (0.78 g, 59.0% yield). ¹H NMR (400 MHz, DMSO-d₆) δ=1.1.98 (s, 1H), 8.05-8.07 (m, 1H), 7.53-7.54 (m, 1H), 6.99-7.09 (m, 1H), 6.80-6.88 (m, 2H), 6.33-6.40 (m, 1H), 5.16 (s, 2H). LC-MS: 278.1 [M−H].

### Intermediate IB-D2

### 4-((1H-pyrrolo[2,3-b]pyridin-4-yl)oxy)-2-fluoroaniline

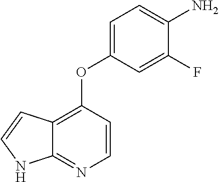 [SMILES: Nc1ccc(Oc2ccnc3[nH]ccc23)cc1F]

The title compound was obtained following a similar procedure described for Intermediate Ib-D1, starting from (2-fluoro-4-((1-((2-(trimethylsilyl)ethoxy)methyl)-1H-pyrrolo[2,3-b]pyridin-4-yl)oxy)phenyl)carbamate (Ib-C1, 0.100 g, 0.21 mmol), TFA (0.5 mL), and K₂CO₃ (0.087 g, 0.63 mmol), and was obtained as a brown gum (0.055 g) which was used without further purification. LC-MS: 244.1 [M+H].

### Intermediate IB-D3

### 2-fluoro-4-((3-methyl-1H-pyrrolo[2,3-b]pyridin-4-yl)oxy)aniline

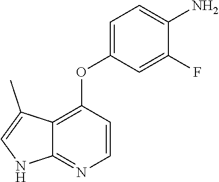 [SMILES: Cc1c[nH]c2nccc(Oc3ccc(N)c(F)c3)c12]

The title compound was obtained following a similar procedure described for Intermediate Ib-D1, starting from tert-butyl (2-fluoro-4-((3-methyl-1-((2-(trimethylsilyl)ethoxy)methyl)-1H-pyrrolo[2,3-b]pyridin-4-yl)oxy)phenyl)carbamate (Ib-C3, 0.30 g, 0.6 mmol), TFA (5 mL), and K₂CO₃ (0.25 g, 1.8 mmol), and was obtained as a brown gum (0.20 g) which was used without further purification. LC-MS: 258.1 [M+H].

### Intermediate IB-D4

### 4-((3-cyclopropyl-1H-pyrrolo[2,3-b]pyridin-4-yl)oxy)-2-fluoroaniline

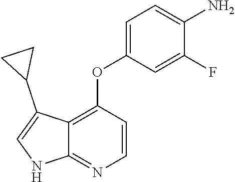 [SMILES: Nc1ccc(Oc2ccnc3[nH]cc(C4CC4)c23)cc1F]

The title compound was obtained following a similar procedure described for Intermediate Ib-D1, starting from tert-butyl (4-((3-cyclopropyl-1-((2-(trimethylsilyl)ethoxy)methyl)-1H-pyrrolo[2,3-b]pyridin-4-yl)oxy)-2-fluorophenyl)carbamate (Ib-C4, 0.070 g, 0.136 mmol), TFA (1 mL), and K₂CO₃ (0.019 g, 0.136 mmol), and was obtained as a brown gum (0.035 g) which was used without further purification. LC-MS: 284.0 [M+H].

### Intermediate IB-D5

### 4-((3-chloro-1H-pyrrolo[2,3-b]pyridin-4-yl)oxy)-3-fluoroaniline

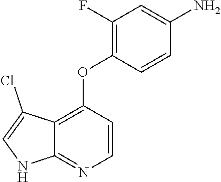 [SMILES: Nc1ccc(Oc2ccnc3[nH]cc(Cl)c23)c(F)c1]

The title compound was obtained following a similar procedure described for Intermediate Ib-D1, starting from tert-butyl (4-((3-chloro-1-((2-(trimethylsilyl)ethoxy)methyl)-1H-pyrrolo[2,3-b]pyridin-4-yl)oxy)-3-fluorophenyl)carbamate (Ib-C6, 0.110 g, 0.216 mmol). TFA (2 mL), and K₂CO₃ (0.089 g, 0.650 mmol), and was obtained as a brown gum (0.065 g) which was used without further purification. LC-MS: 277.9 [M+H].

### Intermediate IB-D6

### 4-((1H-pyrrolo[2,3-b]pyridin-4-yl)oxy)-3-(trifluoromethyl)aniline

### Step 1: Synthesis of 4-(4-nitro-2-(trifluoromethyl)phenoxy)-1H-pyrrolo[2,3-b]pyridine

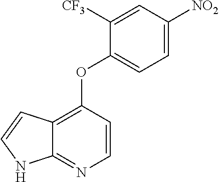 [SMILES: O=[N+]([O-])c1ccc(Oc2ccnc3[nH]ccc23)c(C(F)(F)F)c1]

K₂CO₃ (4.11 g, 29.8 mmol) was added to a solution of 1-fluoro-4-nitro-2-(trifluoromethyl)benzene (1.69 g, 8.12 mmol) and 1H-pyrrolo[2,3-b]pyridin-4-ol (1.00 g, 7.45 mmol) in DMSO (5 mL) and the resulting mixture was stirred at 25° C. for 1 h. Following completion of the reaction (as indicated by TLC), the reaction mixture was poured into ice-cold water (50 mL) and extracted with EtOAc (3×25 mL). The combined organic phases were washed with water (20 mL) and brine (20 mL), dried over Na₂SO₄, filtered, and concentrated under reduced pressure to give crude material which was purified by flash chromatography (silica gel 230-400 mesh), affording the title compound as a yellow solid (0.72 g, 30% yield). ¹H NMR (400 MHz, DMSO-d₆) δ 12.05 (bs, 1H), 8.59 (s, 1H), 8.45-8.59 (m, 1H), 8.29 (d, J=5.2 Hz, 1H), 7.48-7.50 (m, 1H), 7.26 (d, J=9.2 Hz, 1H), 6.93 (d, J=5.2 Hz, 1H), 6.11-6.12 (m, 1H). LC-MS: 324.0 [M+H].

### Step 2: Synthesis of 4-((1H-pyrrolo[2,3-b]pyridin-4-yl)oxy-3-(trifluoromethyl)aniline

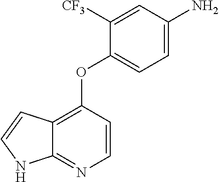 [SMILES: Nc1ccc(Oc2ccnc3[nH]ccc23)c(C(F)(F)F)c1]

Iron powder (1.20 g, 21.65 mmol) and ammonium chloride (1.15 g, 21.65 mmol) were added to a solution of 4-(4-nitro-2-(trifluoromethyl)phenoxy)-1H-pyrrolo[2,3-b]pyridine (0.70 g, 2.165 mmol) in ethanol (20 mL) and water (8 mL) and the resulting suspension was stirred at 80° C. for 3 h. Following completion of the reaction (as indicated by TLC), the reaction mixture was filtered through a pad of of diatomaceous earth which was then rinsed with EtOAc (2.15 mL). The combined filtrates were concentrated under reduced pressure to give a residue which was dissolved in EtOAc (25 mL), washed with brine (5 mL), dried over Na₂SO₄, filtered, and concentrated under reduced pressure to yield the title compound as a brown solid (0.65 g) which was used without further purification. ¹H NMR (400 MHz, DMSO-d₆) δ=11.70 (bs, 1H), 8.05 (d, J=5.6 Hz, 1H), 7.32-7.33 (n, 1H), 6.97-7.04 (m, 2H), 6.85 (d, J=11.2 Hz, 1H), 6.32-6.34 (m, 1H), 6.12 (d, J=5.6 Hz, 1H), 5.59 (bs, 2H) LC-MS: 294.1 [M+H].

### Intermediate IB-D7

### 4-((3-chloro-1H-pyrrolo[2,3-b]pyridin-4-yl)oxy)-2,6-difluoroaniline

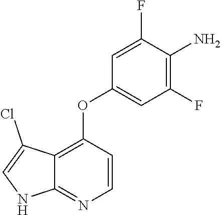 [SMILES: Nc1c(F)cc(Oc2ccnc3[nH]cc(Cl)c23)cc1F]

The title compound was obtained following a similar procedure described for intermediate Ib-D1, starting from tert-butyl (4-((3-chloro-1-((2-(trimethylsilyl)ethoxy)methyl)-1H-pyrrolo[2,3-b]pyridin-4-yl)oxy)-2,6-difluorophenyl)carbamate (Ib-C8, 0.16 g, 0.3 mmol). TFA (1.5 mL), and K₂CO₃ (0.20 g), and was obtained as a brown gum (0.05 g) which was used without further purification. LC-MS: 296.0 [M+H].

### Intermediate IB-D8

### 4-((3-chloro-1H-pyrrolo[2,3-b]pyridin-4-yl)oxy)-2,5-difluoroaniline

### Step 1: Synthesis of 4-(2,5-difluoro-4-nitrophenoxy)-1H-pyrrolo[2,3-b]pyridine

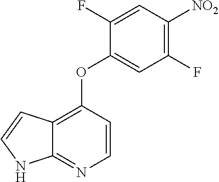 [SMILES: O=[N+]([O-])c1cc(F)c(Oc2ccnc3[nH]ccc23)cc1F]

K₂CO₃ (0.515 g, 3.730 mmol) was added to a solution of 1H-pyrrolo[2,3-b]pyridin-4-ol (0.250 g, 1.864 mmol) and 1,2,4-trifluoro-5-nitrobenzene (0.660 g, 3.730 mmol) in DMF (5 mL) and the resulting mixture was stirred at 25° C. for 12 h. Following completion of the reaction (as indicated by TLC), the reaction mixture was poured into crushed ice (50 g) and extracted with DCM (2×20 mL). The combined organic phases were washed with water (20 mL), dried over Na₂SO₄, filtered, and concentrated under reduced pressure to give crude material which was purified by flash chromatography (silica gel 230-400 mesh, eluting with 60% EtOAc in petroleum ether) to yield the title compound as a yellow solid (0.10 g, 14% yield). ¹H NMR (400 MHz, DMSO-d₆) δ=11.98 (bs, 1H), 8.43-8.47 (m, 1H), 8.21 (d, J=5.2 Hz, 1H), 7.58-7.63 (m, 1H), 7.48-7.50 (m, 1H), 6.79 (d, J=5.2 Hz, 1H), 6.30-6.32 (m, 1H). LC-MS: 292.1 [M+H].

### Step 2: Synthesis of 4-(2,5-difluoro-4-nitrophenoxy)-1-((2-(trimethylsilyl)ethoxy)methyl)-1H-pyrrolo[2,3-]pyridine

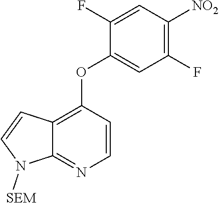 [SMILES: C[Si](C)(C)CCOCn1ccc2c(Oc3cc(F)c([N+](=O)[O-])cc3F)ccnc21]

DIPEA (0.108 mL, 0.618 mmol) and SEM-Cl (0.085 mL, 0.481 mmol) were added to a solution of 4-(2,5-difluoro-4-nitrophenoxy)-1H-pyrrolo[2,3-b]pyridine (0.10 g, 0.343 mmol) in acetonitrile (5 mL) at 0° C. and the resulting mixture was stirred at 25° C. for 12 h. Following completion of the reaction (as indicated by UPLC and TLC), the reaction mixture was directly concentrated under reduced pressure to yield crude material which was purified by Isolera (silica gel 230-400 mesh, eluting with 3% EtOAc in petroleum ether) to yield the title compound as a colorless liquid (0.08 g, 54% yield). ¹H NMR (400 MHz, DMSO-d₆) δ=8.44-8.49 (m, 1H), 8.28 (d, J=5.2 Hz, 1H), 7.63-7.68 (m, 2H), 6.86 (d, J=5.2 Hz, 1H), 6.43 (d, 1=3.6 Hz, 1H), 5.66 (s, 2H), 3.54 (t, J=8.0 Hz, 2H), 0.83 (t, J=8.0 Hz, 2H), −0.09 (s, 9H). LC-MS: 422.1 [M+H].

### Step 3: Synthesis of 3-chloro-4-(2,5-difluoro-4-nitrophenoxy)-1-((2-(trimethylsilyl)ethoxy)methyl)-1H-pyrrolo[2,3-b]pyridine

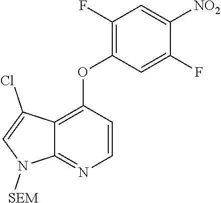 [SMILES: C[Si](C)(C)CCOCn1cc(Cl)c2c(Oc3cc(F)c([N+](=O)[O-])cc3F)ccnc21]

NCS (0.075 g, 0.561 mmol) was added to a solution of 4-(2,5-difluoro-4-nitrophenoxy)-1-((2-(trimethylsilyl)ethoxy)methyl)-1H-pyrrolo[2,3-b]pyridine (0.215 g, 0.510 mmol) in DMF (10 mL) and the resulting mixture was stirred at 25° C. for 12 h. Following completion of the reaction (as indicated by TLC), the reaction mixture was poured into crushed ice (100 g) and extracted with DCM (2.25 mL). The combined organic phases were washed with water (5 mL) and brine (5 mL), dried over Na₂SO₄, filtered, and concentrated under reduced pressure to yield the title compound as an off-white solid (0.3 g) which was used without further purification LC-MS: 456.0 [M+H].

### Step 4: Synthesis of 4-((3-chloro-1-((2-(trimethylsilyl)ethoxy)methyl)-1H-pyrrolo[2,3-b]pyridin-4-yl)oxy)-2,5-difluoroamine

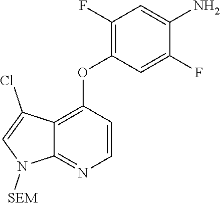 [SMILES: C[Si](C)(C)CCOCn1cc(Cl)c2c(Oc3cc(F)c(N)cc3F)ccnc21]

Iron powder (0.388 g, 6.95 mmol) and ammonium chloride (0.372 g, 6.95 mmol) were added to a solution of 3-chloro-4-(2,5-difluoro-4-nitrophenoxy)-1-((2-(trimethylsilyl)ethoxy)methyl)-1H-pyrrolo[2,3-b]pyridine (0.317 g, 0.695 mmol) in ethanol (5 mL) and water (2 mL) and the resulting suspension was stirred at 80° C. for 3 h. Following completion of the reaction (as indicated by TLC), the reaction mixture was filtered through a pad of diatomaceous earth which was then rinsed with EtOAc (2×5 mL). The combined filtrates were concentrated under reduced pressure to give a residue which was taken in EtOAc (25 mL), washed with brine (5 mL), dried over Na₂SO₄, filtered, and concentrated under reduced pressure, affording the title compound as a brown gum (0.26 g) which was used without further purification. LC-MS: 426.0 [M+H].

### Step 5: Synthesis of 4-((3-chloro-1H-pyrrolo[2,3-b]pyridin-4-yl)oxy)-2,5-difluoroaniline

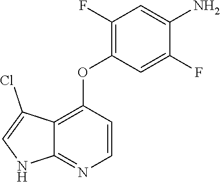 [SMILES: Nc1cc(F)c(Oc2ccnc3[nH]cc(Cl)c23)cc1F]

The title compound was obtained following a similar procedure described for Intermediate Ib-D1, starting from 4-((3-chloro-1-((2-(trimethylsilyl)ethoxy)methyl)-1H-pyrrolo[2,3-b]pyridin-4-yl)oxy)-2,5-difluoroaniline (0.26 g, 0.610 mmol), TFA (0.8 mL), and K₂CO₃ (0.25 g), and was obtained as a brown solid (0.07 g) which was used without further purification. LC-MS: 296.0 [M+H].

### Intermediate IB-D9

### 4-((3-chloro-1H-pyrrolo[2,3-b]pyridin-4-yl)oxy)cyclohexan-1-amine

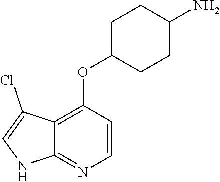 [SMILES: NC1CCC(Oc2ccnc3[nH]cc(Cl)c23)CC1]

The title compound was obtained following a similar procedure described for Intermediate Ib-D1, starting from tert-butyl (4-(3-chloro-1-((2-(trimethylsilyl)ethoxy)methyl)-1H-pyrrolo[2,3-b]pyridin-4-yl)oxy)cyclohexyl)carbamate (Ib-C10, 0.080 g, 0.161 mmol), TFA (0.24 mL), and K₂CO₃ (0.067 g), and was obtained as a pale brown solid (0.035 g, 70% yield). LC-MS: 266.2 [M+H].

### Intermediate IB-D10

### 4-((1H-pyrrolo[2,3-b]pyridin-4-yl)oxy)cyclohexan-1-amine

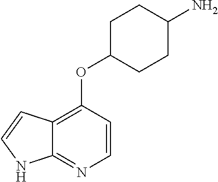 [SMILES: NC1CCC(Oc2ccnc3[nH]ccc23)CC1]

TFA (0.035 mL, 0.453 mmol) was added dropwise to a stirred solution of tert-butyl (4-((1H-pyrrolo[2,3-b]pyridin-4-yl)oxy)cyclohexyl)carbamate (step 2 of Intermediate Ib-C9, 0.150 g, 0.453 mmol) in DCM (5 mL) at 0° C. and the resulting mixture was stirred at 25° C. for 3 h. Following completion of the reaction (as indicated by UPLC), the reaction mixture was concentrated under reduced pressure to afford the title compound (0.20 g) which was used without further purification. LC-MS: 232.1 [M+H].

### Intermediate IB-D11

### 4-((3-chloro-2-methyl-1H-pyrrolo[2,3-b]pyridin-4-yl)oxy)-2-fluoroaniline

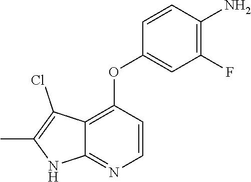 [SMILES: Cc1[nH]c2nccc(Oc3ccc(N)c(F)c3)c2c1Cl]

The title compound was obtained following a similar procedure described for Intermediate Ib-D1, starting from tert-butyl (4-((3-chloro-2-methyl-1-((2-(trimethylsilyl)ethoxy)methyl)-1H-pyrrolo[2,3-b]pyridin-4-yl)oxy)-2-fluorophenyl)carbamate (Ib-C12, 0.110 g, 0.211 mmol), TFA (0.5 mL), and K₂CO₃ (0.087 g), and was obtained as an off-white solid (0.07 g, 86% yield). LC-MS: 292.0 [M+H].

### Intermediate IB-D12

### 2-fluoro-4-((2-methyl-1H-pyrrolo[2,3-b]pyridin-4-yl)oxy)aniline

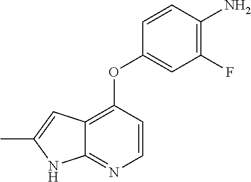 [SMILES: Cc1cc2c(Oc3ccc(N)c(F)c3)ccnc2[nH]1]

The title compound was obtained following a similar procedure described for Intermediate Ib-D1, starting from tert-butyl (2-fluoro-4-((2-methyl-1-((2-(trimethylsilyl)ethoxy)methyl)-1H-pyrrolo[2,3-b]pyridin-4-yl)oxy)phenyl)carbamate (Ib-C11, 0.151 g, 0.3 mmol), TFA (0.6 mL), and K₂CO₃ (0.120 g), and was obtained as an off-white solid (0.057 g) which was used without further purification LC-MS: 258.1 [M+H].

### Intermediate IB-D13

### 4-((5-chloro-7H-pyrrolo[2,3-b]pyrimidin-4-yl)oxy)-2-fluoroaniline

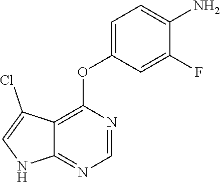 [SMILES: Nc1ccc(Oc2ncnc3[nH]cc(Cl)c23)cc1F]

The title compound was obtained following a similar procedure described for Intermediate Ib-D1, starting from tert-butyl (4-((5-chloro-7-((2-(trimethylsilyl)ethoxy)methyl)-7H-pyrrolo[2,3-d]pyrimidin-4-yl)oxy)-2-fluorophenyl)carbamate (Ib-C14, 0.100 g, 0.196 mmol), TFA (0.5 mL), and K₂CO₃ (0.081 g, 0.590 mmol), and was obtained as a brown gum (0.050 g) which was used without further purification. LC-MS: 278.9 [M+H].

### Intermediate D14

### 4-((7H-pyrrolo[2,3-d]pyrimidin-4-yl)oxy)-2-fluoroaniline

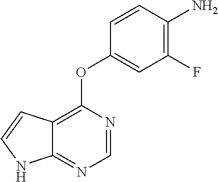 [SMILES: Nc1ccc(Oc2ncnc3[nH]ccc23)cc1F]

The title compound was obtained following a similar procedure described for Intermediate Ib-D1, starting from tert-butyl (2-fluoro-4-((7-((2-(trimethylsilyl)ethoxy)methyl)-7H-pyrrolo[2,3-d]pyrimidin-4-yl)oxy)phenyl)carbamate (Ib-C13, 0.20 g, 0.42 mmol), TFA (1 mL), and K₂CO₃ (0.17 g, 1.26 mmol), and was obtained as a brown gum (0.10 g) which was used without further purification. LC-MS: 245.1 [M+H].

### Intermediate IB-D15

### 2-fluoro-4-((2-methyl-3H-imidazo[4,5-b]pyrimidin-7-yl)oxy)aniline

### Step 1: Synthesis of tert-butyl (4-((2-amino-3-nitropyridin-4-yl)oxy)-2-fluorophenyl)carbamate

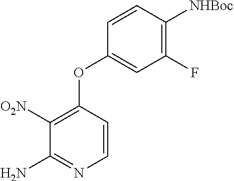 [SMILES: CC(C)(C)OC(=O)Nc1ccc(Oc2ccnc(N)c2[N+](=O)[O-])cc1F]

Potassium tert-butoxide (0.194 g, 1.729 mmol) was added to a stirred solution of tert-butyl (2-fluoro-4-hydroxyphenyl)carbamate (Ib-B1, 0.360 g, 1.585 mmol) in DMF (7 mL) at 25° C. and the resulting mixture was purged with N₂ for 1 h. A solution of 4-chloro-3-nitropyridin-2-amine (0.250 g, 1.440 mmol) in DMF was then added and the resulting mixture was stirred at 70° C. for 20 h. Following completion of the reaction (as indicated by UPLC and TLC), the reaction mixture was diluted with EtOAc (10 mL) and washed with ice-cold water (10 mL) and brine (10 mL). The organic layer was dried over Na₂SO₄, filtered, and concentrated under reduced pressure to yield crude material which was purified by flash chromatography (silica gel 230-400 mesh, eluting with 30% EtOAc in petroleum ether), affording the title compound as an off white solid (0.326 g, 62% yield). ¹H NMR (400 MHz, DMSO-d₆) δ=9.09 (bs, 1H), 8.03 (d, J=5.6 Hz, 1H), 7.64-7.68 (m, 1H), 7.23-7.26 (m, 3H), 7.01 (d, J=12.4 Hz, 1H), 6.03 (d, J=5.6 Hz, 1H), 1.47 (s, 9H). LC-MS: 365.1 [M+H].

### Step 2: Synthesis of tert-butyl (4-((2,3-diaminopyridin-4-yl)oxy)-2-fluorophenyl)carbamate

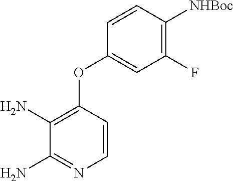 [SMILES: CC(C)(C)OC(=O)Nc1ccc(Oc2ccnc(N)c2N)cc1F]

The title compound was obtained following a similar procedure described for step 4 of Intermediate Ib-D8, starting from tert-butyl (4-((2-amino-3-nitropyridin-4-yl)oxy)-2-fluorophenyl)carbamate (0.326 g, 0.895 mmol), iron powder (0.500 g, 8.950 mmol), and ammonium chloride (0.479 g, 8.950 mmol), and was obtained as an off-white solid (0.29 g) which was used without further purification. ¹H NMR (400 MHz. DMSO-d₆) δ=8.98 (bs, 1H), 7.40-7.56 (m, 1H), 7.30 (d, J=8.4 Hz, 1H), 7.04-7.08 (m, 1H), 6.96 (bs, 2H), 6.88 (d, J=10.4 Hz, 1H), 6.27 (d, J=8.00 Hz, 1H), 5.24 (bs, 2H), 1.45 (s, 9H). LC-MS: 335.2 [M+H].

### Step 3: Synthesis of 2-fluoro-4-((2-methyl-3H-imidazo[4,5-b]pyridin-7-yl)oxy)aniline

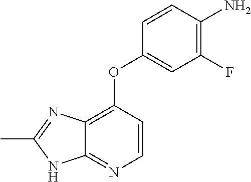 [SMILES: Cc1nc2c(Oc3ccc(N)c(F)c3)ccnc2[nH]1]

Polyphosphoric acid (0.042 ml, 0.888 mmol) was added to a solution of tert-butyl (4-((2,3-diaminopyridin-4-yl)oxy)-2-fluorophenyl)carbamate (0.297 g, 0.888 mmol) in acetic acid (0.076 ml, 1.332 mmol) and the resulting mixture was stirred at 150° C. for 2 h. Following completion of the reaction (as indicated by UPLC), the reaction mixture was concentrated under reduced pressure to give a residue which was basified with NaOH (10% solution) to pH=13) and extracted with EtOAc (2×15 mL). The combined organic phases were washed with brine (10 mL), dried over Na₂SO₄, filtered, and concentrated under reduced pressure to afford the title compound (0.085 g) which was used without further purification. LC-MS: 259.0 [M+H].

### General Procedure for the Synthesis of Carbamate Intermediates Ib-E:

Pyridine (1.2 eq) and phenyl chloroformate (1.5 eq) were added to a solution of aminopyrazole (1.0 eq) in THF (10 vol) at 0° C. The reaction mixture was allowed to warm to 25° C. and was stirred for 12 h. After completion of the reaction (monitored by TLC), the mixture was diluted with EtOAc (10 mL) and washed with brine (5 mL). The organic layer was dried over Na₂SO₄, filtered, and concentrated under reduced pressure to yield crude material which was purified by Isolera (silica gel 230-400 mesh, eluting with 10% to 20% EtOAc in petroleum ether), affording the desired carbamate which was used without further purification.

The following carbamates were prepared using the above general procedure.

| | Carbamate structure | Amine structure | |
|---|---|---|---|
| Intermediate | (product) | (starting material) | LC-MS |
| Ib-E1 | 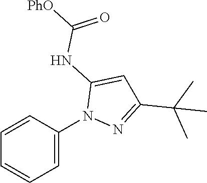 [SMILES: CC(C)(C)c1cc(NC(=O)Oc2ccccc2)n(-c2ccccc2)n1] | 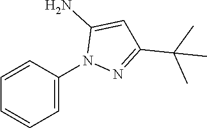 | 336.1 [M + H] |
| Ib-E2 | 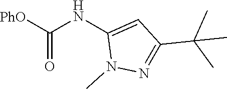 [SMILES: Cn1nc(C(C)(C)C)cc1NC(=O)Oc1ccccc1] | 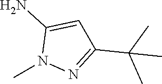 | 274.1 [M + H] |
| Ib-E3 | 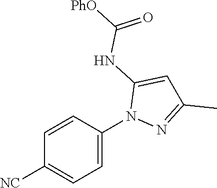 [SMILES: Cc1cc(NC(=O)Oc2ccccc2)n(-c2ccc(C#N)cc2)n1] | 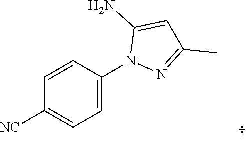 | 319.1 [M + H] |
| Ib-E4 | 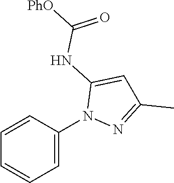 [SMILES: Cc1cc(NC(=O)Oc2ccccc2)n(-c2ccccc2)n1] | 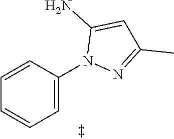 | 294.0 [M + H] |
| Ib-E5 | 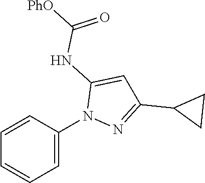 [SMILES: O=C(Nc1cc(C2CC2)nn1-c1ccccc1)Oc1ccccc1] | 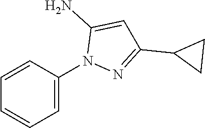 [SMILES: Nc1cc(C2CC2)nn1-c1ccccc1] | 320.2 [M + H] |
| Ib-E6 | 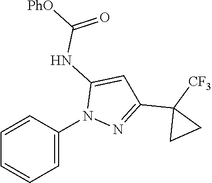 [SMILES: O=C(Nc1cc(C2(C(F)(F)F)CC2)nn1-c1ccccc1)Oc1ccccc1] | 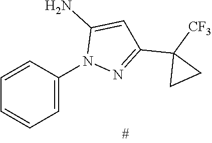 [SMILES: Nc1cc(C2(C(F)(F)F)CC2)nn1-c1ccccc1] | 388.1 [M + H] |
| Ib-E7 | 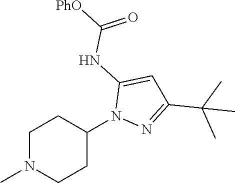 [SMILES: CN1CCC(n2nc(C(C)(C)C)cc2NC(=O)Oc2ccccc2)CC1] | 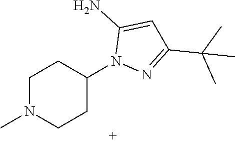 | 357.4 [M + H] |

Note:
Amines used for the synthesis of carbamates are either commercially available or were synthesized using literature procedures.
† compound synthesized as reported in Tetrahedron Lett. 2019, 60(1), 72-74
‡ compound synthesized as reported in Bioorg. Med Chem. Lett 2019, 29(16), 2124-2128.
# compound synthesized as synthesized as reported in PCT Publication No. WO 2009/117080 A1.
+ compound synthesized as reported in US Pub. No. 2015/0141601 A1.

### General Procedure for the Synthesis of Examples Ib-1 Through Ib-21

Triethylamine (2.0 eq) was added to a mixture of amine intermediate (Ib-D1 through Ib-D15, 1.0 eq) and carbamate intermediate (Ib-E1 through Ib-E7, 1.0 eq) in THF (5 mL) and the resulting solution was heated to 60° C. for 12 h. Following completion of the reaction (as indicated by LC-MS), the reaction mixture was concentrated under reduced pressure to yield crude material which was purified by RP-HPLC, affording the title compounds.

### Example IB-1

### 1-(4-((1H-pyrrolo[2,3-b]pyridin-4-yl)oxy)-2-fluorophenyl)-3-(3-(tert-butyl)-1-phenyl-1H-pyrazol-5-yl)urea

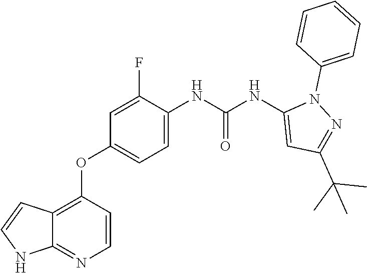 [SMILES: CC(C)(C)c1cc(NC(=O)Nc2ccc(Oc3ccnc4[nH]ccc34)cc2F)n(-c2ccccc2)n1]

The title compound was obtained following the general procedure described above, starting from 4-((1H-pyrrolo[2,3-b]pyridin-4-yl)oxy)-2-fluoroaniline (Ib-D2, 0.055 g, 0.23 mmol) and phenyl (3-(tert-butyl)-1-phenyl-1H-pyrazol-5-yl)carbamate (Ib-E1, 0.076 g, 0.23 mmol), and was obtained as an off-white solid (14 mg, 13% yield). ¹H NMR (400 MHz, DMSO-d₆) δ=11.77 (s, 1H), 9.14 (s, 1H), 9.05 (s, 1H), 8.07-8.12 (m, 2H), 7.53-7.58 (m, 4H), 7.36-7.49 (m, 2H), 7.19-7.23 (m, 1H), 6.98-7.01 (m, 1H), 6.46-6.47 (m, 1H), 6.39 (s, 1H), 6.20-6.22 (m, 1H), 1.28 (s, 9H). LC-MS: 485.1 [M+H].

### Example IB-2

### 1-(4-((1H-pyrrolo[2,3-]pyridin-4-yl)oxy)-3-(TRIFLUOROMETHYL)phenyl)-3-(3-(tert-butyl)-1-phenyl-1H-pyrazol-5-yl)urea

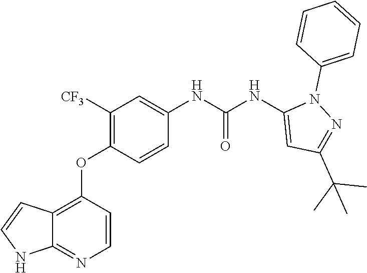 [SMILES: CC(C)(C)c1cc(NC(=O)Nc2ccc(Oc3ccnc4[nH]ccc34)c(C(F)(F)F)c2)n(-c2ccccc2)n1]

The title compound was obtained following the general procedure described for Example Ib-1, starting from 4-((1H-pyrrolo[2,3-b]pyridin-4-yl)oxy)-3-(trifluoromethyl)aniline (Ib-D6, 0.100 g, 0.34 mmol) and phenyl (3-(tert-butyl)-1-phenyl-1H-pyrazol-5-yl)carbamate (Ib-E1, 0.114 g, 0.34 mmol), and was obtained as a reddish-brown solid (35 mg, 19% yield). ¹H NMR (400 MHz, DMSO-d₆) δ=11.80 (bs, 1H), 9.72 (bs, 1H), 8.84 (bs, 1H), 8.08-8.10 (m, 2H), 7.51-7.60 (m, 5H), 7.36-7.43 (m, 2H), 7.23 (d, J=9.2 Hz, 1H), 6.46 (d, J=5.6 Hz, 1H), 6.39 (s, 1H), 6.19-6.22 (m, 1H), 1.30 (s, 9H). LC-MS: 535.2 [M+H].

### Example IB-3

### 1-(4-((7H-pyrrolo[2,3-d]pyrimidin-4-yl)oxy)-2-fluorophenyl)-3-(3-(tert-butyl)-1-phenyl-1H-pyrazol-5-yl)urea

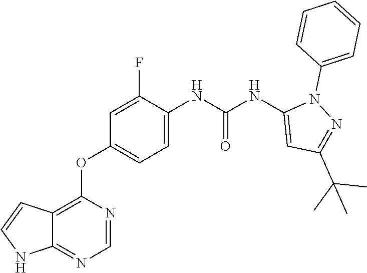 [SMILES: CC(C)(C)c1cc(NC(=O)Nc2ccc(Oc3ncnc4[nH]ccc34)cc2F)n(-c2ccccc2)n1]

The title compound was obtained following the general procedure described for Example Ib-1, starting from 4-(7H-pyrrolo[2,3-d]pyrimidin-4-yl)oxy)-2-fluoroaniline (Ib-D14, 0.100 g, 0.41 mmol) and phenyl (3-(tert-butyl)-1-phenyl-1H-pyrazol-5-yl)carbamate (Ib-E1, 0.137 g, 0.41 mmol), and was obtained as an off-white solid (46 mg, 23% yield). ¹H NMR (400 MHz, DMSO-d₆) δ=12.28 (bs, 1H), 9.63 (bs, 1H), 8.31 (s, 1H), 7.97-8.02 (m, 1H), 7.47-7.59 (m, 61), 7.37-7.41 (m, 1H), 7.25-7.29 (m, 1H), 7.03-7.05 (m, 1H), 6.49 (s, 1H), 6.35-6.35 (m, 1H), 1.29 (s, 9H). LC-MS: 486.2 [M+H].

### Example IB-4

### 1-(3-(tert-butyl)-1-phenyl-1H-pyrazol-5-yl)-3-(2-fluoro-4-((3-methyl-1H-pyrrolo[2,3-b]pyridin-4-yl)oxy)phenyl)urea

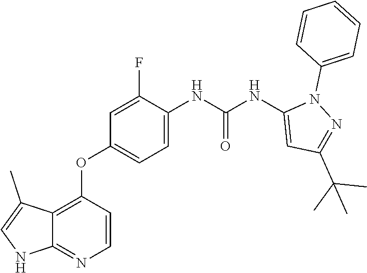 [SMILES: Cc1c[nH]c2nccc(Oc3ccc(NC(=O)Nc4cc(C(C)(C)C)nn4-c4ccccc4)c(F)c3)c12]

The title compound was obtained following the general procedure described for Example Ib-1, starting from 2-fluoro-4-((3-methyl-1H-pyrrolo[2,3-b]pyridin-4-yl)oxy)aniline (Ib-D3, 0.126 g, 0.49 mmol) and phenyl (3-(tert-butyl)-1-phenyl-1H-pyrazol-5-yl)carbamate (Ib-E1, 0.160 g, 0.49 mmol), and was obtained as an off-white solid (27 mg, 11% yield). ¹H NMR (400 MHz, DMSO-d₆) δ=11.40 (bs, 1H), 8.99 (bs, 1H), 8.88 (bs, 1H), 8.07-8.12 (m, 1H), 8.02-8.03 (m, 1H), 7.52-7.58 (m, 4H), 7.42-7.45 (m, H), 7.14-7.19 (m, 2H), 6.94-6.97 (m, 1H), 6.40 (s, 1H), 6.32 (d, J=5.2 Hz, 1H), 2.32 (s, 3H), 1.28 (s, 9H). LC-MS: 499.1 [M+H].

### Example IB-5

### 1-(3-(tert-butyl)-1-phenyl-1H-pyrazol-5-yl)-3-(2-fluoro-4-((2-methyl-1H-pyrrolo[2,3-b]pyridin-4-yl)oxy)phenyl)urea

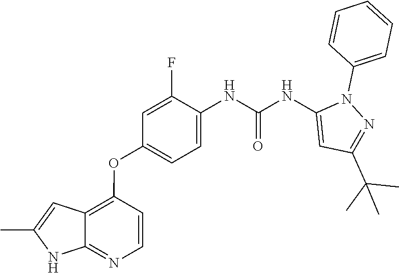 [SMILES: Cc1cc2c(Oc3ccc(NC(=O)Nc4cc(C(C)(C)C)nn4-c4ccccc4)c(F)c3)ccnc2[nH]1]

The title compound was obtained following the general procedure described for Example Ib-1, starting from 2-fluoro-4-((2-methyl-1H-pyrrolo[2,3-b]pyridin-4-yl)oxy)aniline (Ib-D12, 0.057 g, 0.22 mmol) and phenyl (3-(tert-butyl)-1-phenyl-1H-pyrazol-5-yl)carbamate (Ib-E1, 0.074 g, 0.22 mmol), and was obtained as an off-white solid (7 mg, 6% yield). ¹H NMR (400 MHz, DMSO-d₆) δ=11.60 (bs, 1H), 10.56 (bs, 1H), 10.32 (bs, 1H), 7.99 (d, J=5.6 Hz, 1H), 7.85-7.89 (m, 1H), 7.60-7.62 (m, 2H), 7.46-7.50 (m, 2H), 7.32-7.36 (m, 1H), 7.05-7.09 (m, 1H), 6.87-6.89 (m, 1H), 6.46 (d, J=5.6 Hz, 1H), 6.27 (s, 1H), 5.85-5.86 (m, 1H), 2.34 (s, 3H), 1.29 (s, 9H). LC-MS: 499.3 [M+H].

### Example IB-6

### 1-(3-(tert-butyl)-1-phenyl-1H-pyrazol-5-yl)-3-(4-((3-chloro-1H-pyrrolo[2,3-b]pyridin-4-yl)oxy)-2-fluorophenyl)urea

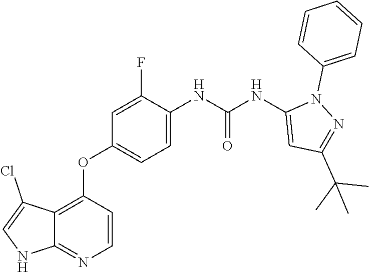 [SMILES: CC(C)(C)c1cc(NC(=O)Nc2ccc(Oc3ccnc4[nH]cc(Cl)c34)cc2F)n(-c2ccccc2)n1]

The title compound was obtained following the general procedure described for Example Ib-1, starting from 4-((3-chloro-1H-pyrrolo[2,3->]pyridin-4-yl)oxy)-2-fluoroaniline (Ib-D1, 0.15 g, 0.54 mmol) and phenyl (3-(tert-butyl)-1-phenyl-1H-pyrazol-5-yl)carbamate (Ib-E1, 0.18 g, 0.54 mmol), and was obtained as an off-white solid (47.74 mg, 27% yield). ¹H NMR (400 MHz, DMSO-d₆) δ=12.08 (s, 1H), 9.02 (s, 1H), 8.90 (s, 1H), 8.11-8.16 (m, 2H), 7.53-7.59 (m, 5H), 7.42-7.46 (m, 1H), 7.22-7.26 (m, 1H), 6.98-7.01 (m, 1H), 6.40-6.41 (m, 2H), 1.29 (s, 9H). LC-MS: 519.2 [M+H].

### Example IB-7

### 1-(3-(tert-butyl)-1-methyl-1H-pyrazol-5-yl)-3-(4-((3-chloro-1H-pyrrolo[2,3-b]pyridin-4-yl)oxy)-2-fluorophenyl)urea

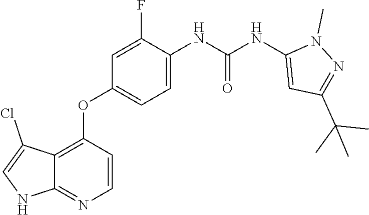 [SMILES: Cn1nc(C(C)(C)C)cc1NC(=O)Nc1ccc(Oc2ccnc3[nH]cc(Cl)c23)cc1F]

The title compound was obtained following the general procedure described for Example Ib-1, starting from 4-((3-chloro-1H-pyrrolo[2,3-b]pyridin-4-yl)oxy)-2-fluoroaniline (Ib-D1, 0.092 g, 0.33 mmol) and phenyl (3-tert-butyl)-1-methyl-1H-pyrazol-5-yl)carbamate (Ib-E2, 0.09 g, 0.33 mmol), and was obtained as an off-white solid (15 mg, 10% yield). ¹H NMR (400 MHz, DMSO-d₆) δ=12.08 (bs, 1H), 8.90 (bs, 1H), 8.83 (bs, 1H), 8.13-8.18 (m, 2H), 7.59 (s, 1H), 7.25-7.29 (m, 1H), 7.00-7.02 (m, 1H), 6.42 (d, J=5.2 Hz, 1H), 6.09 (s, 1H), 3.62 (s, 3H), 1.22 (s, 9H). LC-MS: 457.2 [M+H].

### Example IB-8

### 1-(3-(tert-butyl)-1-phenyl-1H-pyrazol-5-yl)-3-(4-((3-chloro-1H-pyrrolo[2,3-b])pyridin-4-yl)oxy)-2,6-difluorophenyl)urea

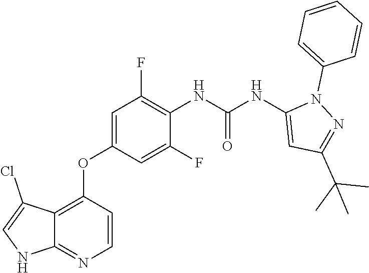 [SMILES: CC(C)(C)c1cc(NC(=O)Nc2c(F)cc(Oc3ccnc4[nH]cc(Cl)c34)cc2F)n(-c2ccccc2)n1]

The title compound was obtained following the general procedure described for Example Ib-1, starting from 4-((3-chloro-1H-pyrrolo[2,3-b]pyridin-4-yl)oxy)-2,6-difluoroaniline (Ib-D7, 0.050 g, 0.16 mmol) and phenyl (3-(tert-butyl)-1-phenyl-1H-pyrazol-5-yl)carbamate (Ib-E1, 0.057 g, 0.16 mmol), and was obtained as an off-white solid (4.4 mg, 7% yield). ¹H NMR (400 MHz, (CD₃OD) δ=8.20 (d, J=5.2 Hz, 1H), 7.54-7.59 (m, 4H), 7.47-7.51 (m, 1H), 7.38 (s, 1H), 6.85-6.88 (m, 2H), 6.68 (d, J=5.6 Hz, 1H), 6.41 (s, 1H), 1.36 (s, 9H). LC-MS: 535.1 [M−H].

### Example IB-9

### 1-(4-((3-chloro-1H-pyrrolo[2,3-b]pyridin-4-yl)oxy)-2-fluorophenyl)-3-(1-(4-cyanophenyl)-3-methyl-1H-pyrazol-5-yl)urea

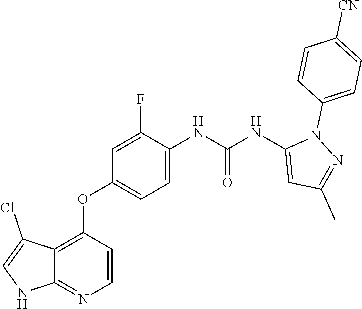 [SMILES: Cc1cc(NC(=O)Nc2ccc(Oc3ccnc4[nH]cc(Cl)c34)cc2F)n(-c2ccc(C#N)cc2)n1]

The title compound was obtained following the general procedure described for Example Ib-1, starting from 4-((3-chloro-1H-pyrrolo[2,3-b]pyridin-4-yl)oxy)-2-fluoroaniline (Ib-D1, 0.075 g, 0.27 mmol) and phenyl (1-(4-cyanophenyl)-3-methyl-1H-pyrazol-5-yl)carbamate (Ib-E3, 0.086 g, 0.27 mmol), and was obtained as an off-white solid (18.7 mg, 14% yield). ¹H NMR (400 MHz, DMSO-d₆) δ=12.08 (bs, 1H), 9.03 (bs, 1H), 8.96 (s, 1H), 8.01-8.14 (m, 4H), 7.79-7.81 (m, 2H), 7.59 (s, 1H), 7.25 (dd, J=2.4, 11.8 Hz, 1H), 6.99-7.02 (m, 1H), 6.41 (d, J=5.2 Hz, 1H), 6.35 (s, 1H), 2.23 (s, 3H). LC-MS: 501.6 [M+H].

### Example IB-10

### 1-(4-((3-chloro-1-pyrrolo[2,3-b]pyridin-4-yl)oxy)-2-fluorophenyl)-3-(3-methyl-1-phenyl-1H-pyrazol-5-yl)urea

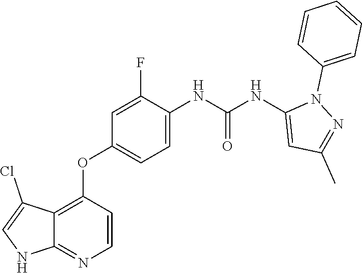 [SMILES: Cc1cc(NC(=O)Nc2ccc(Oc3ccnc4[nH]cc(Cl)c34)cc2F)n(-c2ccccc2)n1]

The title compound was obtained following the general procedure described for Example Ib-1, starting from 4-((3-chloro-1H-pyrrolo[2,3-b]pyridin-4-yl)oxy)-2-fluoroaniline (Ib-D1, 0.07 g, 0.25 mmol) and phenyl (3-methyl-1-phenyl-1H-pyrazol-5-yl)carbamate (Ib-E4, 0.07 g, 0.25 mmol), and was obtained as an off-white solid (4.7 mg, 4% yield). ¹H NMR (400 MHz, CD₃OD) δ=8.10-8.12 (m, 2H), 7.57-7.62 (m, 2H), 7.50-7.53 (m, 3H), 7.34 (s, 1H), 7.03-7.07 (m, 1H), 6.97-7.00 (m, 1H), 6.47 (d, J=5.6 Hz, 1H), 6.41 (s, 1H), 2.31 (s, 3H). LC-MS: 477.1 [M+H].

### Example IB-11

### 1-(4-((3-chloro-1H-pyrrolo[2,3-b]pyridin-4-yl)oxy)-2-fluorophenyl)-3-(3-cyclopropyl-1-phenyl-1H-pyrazol-5-yl)urea

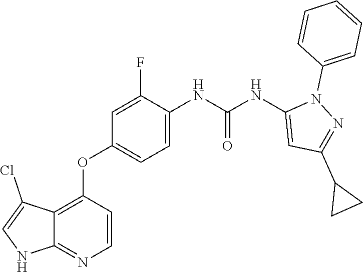 [SMILES: O=C(Nc1ccc(Oc2ccnc3[nH]cc(Cl)c23)cc1F)Nc1cc(C2CC2)nn1-c1ccccc1]

The title compound was obtained following the general procedure described for Example Ib-1, starting from phenyl (4-((3-chloro-1H-pyrrolo[2,3-b]pyridin-4-yl)oxy)-2-fluorophenyl)carbamate (Ib-D1, 0.040 g, 0.144 mmol) and 3-cyclopropyl-1-phenyl-1H-pyrazol-5-amine (Ib-E5, 0.046 g, 0.144 mmol), and was obtained as an off-white solid (3 mg, 4% yield). ¹H NMR (400 MHz, CD₃OD) δ=8.09-8.12 (m, 2H), 7.58-7.61 (m, 2H), 7.50-7.53 (m, 3H), 7.34 (s, 1H), 7.03-7.07 (m, 1H), 6.97-7.00 (m, 1H), 6.46 (d, J=5.6 Hz, 1H), 6.26 (s, 1H), 1.93-1.97 (m, 1H), 0.96-1.00 (m, 2H), 0.78-0.81 (m, 21H). LC-MS: 503.1 [M+H].

### Example IB-12

### 1-(3-(tert-butyl)-1-phenyl-1H-pyrazol-5-yl)-3-(4-((3-chloro-1H-pyrrolo[2,3-b]pyridin-4-yl)oxy)-3-fluorophenyl)urea

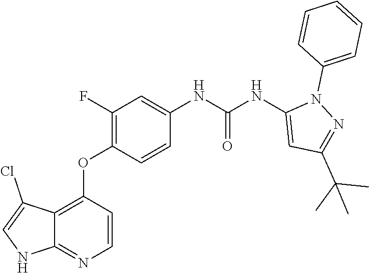 [SMILES: CC(C)(C)c1cc(NC(=O)Nc2ccc(Oc3ccnc4[nH]cc(Cl)c34)c(F)c2)n(-c2ccccc2)n1]

The title compound was obtained following the general procedure described for Example Ib-1, starting from 4-((3-chloro-11H-pyrrolo[2,3-b]pyridin-4-yl)oxy)-3-fluoroaniline (Ib-D5, 0.065 g, 0.23 mmol) and phenyl (3-(tert-butyl)-1-phenyl-1H-pyrazol-5-yl)carbamate (Ib-E1, 0.080 g, 023 mmol), and was obtained as an off-white solid (10.0 mg, 8% yield). ¹H NMR (400 MHz, DMSO-d₆) δ=12.08 (bs, 1H), 9.60 (bs, 1H), 8.80 (bs, 1H), 8.09 (d, J=5.6 Hz, 1H), 7.67-7.71 (m, 1H), 7.52-7.59 (m, 5H), 7.38-7.43 (m, 1H), 7.18-7.34 (m, 2H), 6.38 (s, 1H), 6.24-6.27 (m, 1H), 1.30 (s, 9H). LC-MS: 519.1 [M+H].

### Example IB-13

### 1-(3-(tert-butyl)-1-phenyl-1H-pyrazol-5-yl)-3-(4-((5-chloro-7H-pyrrolo[2,3-b]pyrimidin-4-yl)oxy)-2-fluorophenyl)urea

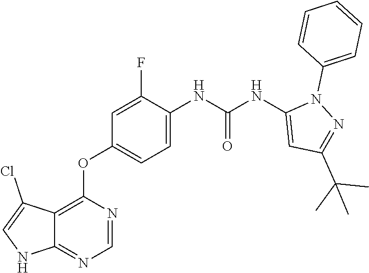 [SMILES: CC(C)(C)c1cc(NC(=O)Nc2ccc(Oc3ncnc4[nH]cc(Cl)c34)cc2F)n(-c2ccccc2)n1]

The title compound was obtained following the general procedure described for Example Ib-1, starting from 4-((5-chloro-7H-pyrrolo[2,3-d]pyrimidin-4-yl)oxy)-2-fluoroaniline (Ib-D13, 0.05 g, 0.179 mmol) and phenyl (3-(tert-butyl)-1-phenyl-1H-pyrazol-5-yl)carbamate (Ib-E1, 0.64 g, 0.179 mmol), and was obtained as an off-white solid (11 mg, 11% yield). ¹H NMR (400 MHz, CD₃OD) δ=8.28 (s, 1H), 8.1.1-815 (m, 1H), 7.58-7.62 (m, 2H), 7.53-7.54 (m, 3H), 7.41 (s, 1H), 7.15-7.18 (m, 1H), 7.05-7.08 (m, 1H), 6.49 (s, 1H), 1.37 (s, 9H). LC-MS: 520.2 [M+H].

### Example IB-14

### 1-(3-tert-butyl)-1-phenyl-1H-pyrazol-5-yl)-3-(4-((3-chloro-2-methyl-1H-pyrrolo[2,3-d]pyridin-4-yl)oxy)-2-fluorophenyl)urea

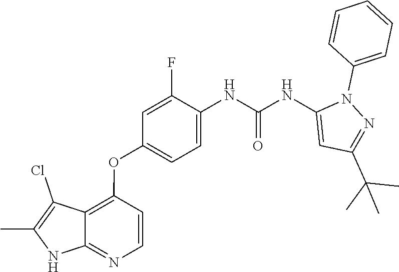 [SMILES: Cc1[nH]c2nccc(Oc3ccc(NC(=O)Nc4cc(C(C)(C)C)nn4-c4ccccc4)c(F)c3)c2c1Cl]

The title compound was obtained following the general procedure described for Example Ib-1, starting from 4-((3-chloro-2-methyl-1H-pyrrolo[2,3-b]pyridin-4-yl)oxy)-2-fluoroaniline (Ib-D11, 0.023 g, 0.079 mmol) and phenyl (3-(tert-butyl)-1-phenyl-1H-pyrazol-5-yl)carbamate (Ib-E1, 0.026 g, 0.079 mmol), and was obtained as an off-white solid (3 mg, 7% yield). ¹H NMR (400 MHz, CD₃OD) δ=8.01-8.08 (m, 2H), 7.52-7.61 (m, 5H), 6.93-7.02 (m, 2H), 6.46-6.48 (m, 2H), 2.42 (s, 3H), 1.37 (s, 9H). LC-MS: 531.1 [M−H].

### Example IB-15

### 1-(3-(methyl)-1-phenyl-1H-pyrazol-5-yl)-3-(4-((3-chloro-1H-pyrrolo[2,3-b]pyridin-4-yl)oxy)-2,5-difluorophenyl)urea

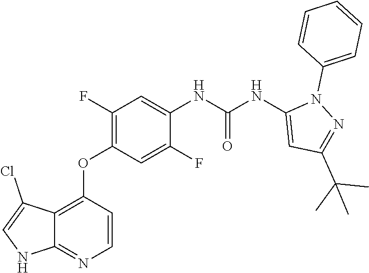 [SMILES: CC(C)(C)c1cc(NC(=O)Nc2cc(F)c(Oc3ccnc4[nH]cc(Cl)c34)cc2F)n(-c2ccccc2)n1]

The title compound was obtained following the general procedure described for Example Ib-1, starting from 4-((3-chloro-1H-pyrrolo[2,3-b]pyridin-4-yl)oxy)-2,5-difluoroaniline (Ib-D8, 0.070 g, 0.237 mmol) and phenyl (3-(tert-butyl)-1-phenyl-1H-pyrazol-5-yl)carbamate (Ib-E1, 0.079 g, 0.237 mmol), and was obtained as an off-white solid (8.3 mg, 6% yield). ¹H NMR (400 MHz, DMSO-d₆) δ=12.10 (bs, 1H), 9.24 (bs, 1H), 8.95 (bs, 1H), 8.20-8.25 (m, 1H), 8.11 (d, J=5.2 Hz, 1H), 7.43-7.60 (m, 6H), 6.43 (s, 1H), 6.36 (d, J=5.2 Hz, 1H), 1.29 (s, 9H). LC-MS: 537.1 [M+H].

### Example IB-16

### 1-(4-((1H-pyrrolo[2,3-b]pyridin-4-yl)oxy)cyclohexyl)-3-(3-(tert-butyl)-1-phenyl-1H-pyrazol-5-yl)urea

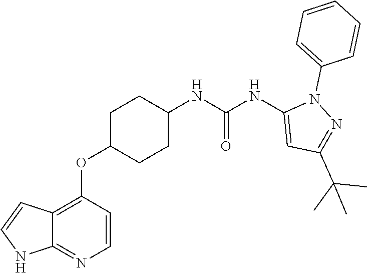 [SMILES: CC(C)(C)c1cc(NC(=O)NC2CCC(Oc3ccnc4[nH]ccc34)CC2)n(-c2ccccc2)n1]

The title compound was obtained following the general procedure described for Example Ib-1, starting from 4-((1H-pyrrolo[2,3-b]pyridin-4-yl)oxy)cyclohexan-1-amine (Ib-D10, 0.100 g, 0.432 mmol) and phenyl (3-(tert-butyl)-1-phenyl-1H-pyrazol-5-yl)carbamate (Ib-E1, 0.145 g, 0.432 mmol), and was obtained as an off-white solid (6.8 mg, 3% yield). ¹H NMR (400 MHz, CD₃OD) δ=8.04-8.05 (m, 1H), 7.46-7.58 (m, 5H), 7.20 (d, J=3.6 Hz, 1H), 6.67 (d, J=5.6 Hz, 1H), 6.50 (d, J=3.6 Hz, 1H), 6.36 (s, 1H), 4.82-4.90 (m, 1H), 3.70-3.74 (m, 1H), 1.92-2.07 (m, 2H), 1.70-1.89 (m, 6H), 1.35 (s, 9H). LC-MS: 473.3 [M+H].

### Example IB-17

### 1-(4-((3-chloro-1H-pyrrolo[2,3-b]pyridin-4-yl)oxy)-2-fluorophenyl)-3-(1-phenyl-3-(1-(trifluoromethyl)cyclopropyl)-1H-pyrazol-5-yl)urea

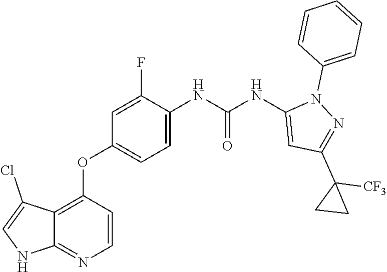 [SMILES: O=C(Nc1ccc(Oc2ccnc3[nH]cc(Cl)c23)cc1F)Nc1cc(C2(C(F)(F)F)CC2)nn1-c1ccccc1]

The title compound was obtained following the general procedure described for Example Ib-1, starting from 4-((3-chloro-1H-pyrrolo[2,3-b]pyridin-4-yl)oxy)-2-fluoroaniline (Ib-D1, 0.073 g, 0.263 mmol) and phenyl (1-phenyl-3-(1-(trifluoromethyl)cyclopropyl)-1H-pyrazol-5-yl)carbamate (Ib-E6, 0.102 g, 0.263 mmol), and was obtained as an off-white solid (25.0 mg, 17% yield). ¹H NMR (400 MHz, DMSO-d₆) δ=9.04 (bs, 1H), 8.99 (bs, 1H), 8.12-8.17 (m, 2H), 7.48-7.61 (m, 6H), 723-7.27 (m, 1H), 6.99-7.02 (m, 1H), 6.61 (s, 1H), 6.40 (d, J=5.6 Hz, 1H), 1.32-1.37 (m, 4H). LC-MS: 569.1 [M+H].

### Example IB-18

### 1-(3-(tert-butyl)-1-phenyl-1H-pyrazol-5-yl)-3-(4-((3-cyclopropyl-1H-pyrrolo[2,3-b]pyridin-4-yl)oxy)-2-fluorophenyl)urea

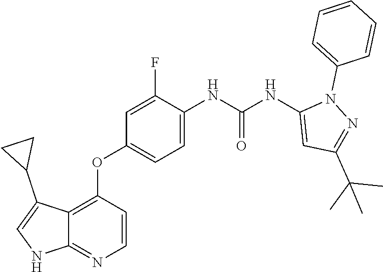 [SMILES: CC(C)(C)c1cc(NC(=O)Nc2ccc(Oc3ccnc4[nH]cc(C5CC5)c34)cc2F)n(-c2ccccc2)n1]

The title compound was obtained following the general procedure described for Example Ib-1, starting from 4-((3-cyclopropyl-1H-pyrrolo[2,3-b]pyridin-4-yl)oxy)-2-fluoroaniline (Ib-D4, 0.040 g, 0.14 mmol) and phenyl (3-(tert-butyl)-1-phenyl-1H-pyrazol-5-yl)carbamate (Ib-E1, 0.047 g, 0.14 mmol), and was obtained as an off-white solid (6.2 mg, 8% yield). ¹H NMR (400 MHz, CD₃OD) δ=8.02-8.08 (m, 2H), 7.57-7.61 (m, 2H), 7.50-7.53 (m, 3H), 6.95-7.03 (m, 3H), 6.48 (s, 1H), 6.45 (d, J=5.6 Hz, 1H), 2.11 (s, 1H), 1.37 (s, 9H), 0.79-0.83 (m, 2H), 0.59-0.62 (m, 2H). LC-MS: 525.2 [M+H].

### Example IB-19

### 1-(3-(tert-butyl)-1-phenyl-1H-pyrazol-5-yl)-3-(2-fluoro-4-((2-methyl-3H-imidazo[4,5-b]pyridin-7-yl)oxy)phenyl)urea

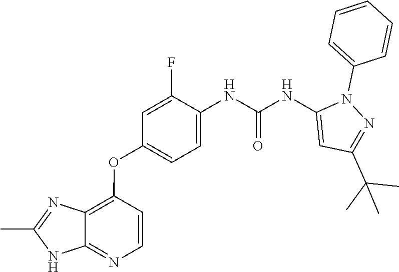 [SMILES: Cc1nc2c(Oc3ccc(NC(=O)Nc4cc(C(C)(C)C)nn4-c4ccccc4)c(F)c3)ccnc2[nH]1]

The title compound was obtained following the general procedure described for Example Ib-1, starting from 2-fluoro-4-((2-methyl-3H-imidazo[4,5-b]pyridin-7-yl)oxy)aniline (Ib-D15, 0.085 g, 0.329 mmol) and phenyl (3-(tert-butyl)-1-phenyl-1H-pyrazol-5-yl)carbamate (Ib-E1, 0.1.10 g, 0.329 mmol), and was obtained as an off-white solid (3.2 mg, 2% yield). ¹H NMR (400 MHz, CD₃OD) δ=8.12-8.19 (m, 2H), 7.50-7.62 (m, 5H), 7.03-7.14 (m, 2H), 6.68 (d, J=5.6 Hz, 1H), 6.48 (s, 1H), 2.66 (s, 3H), 1.37 (s, 9H) LC-MS: 501.1 [M+H].

### Example IB-20

### 1-(3-(tert-butyl)-1-phenyl-1H-pyrazol-5-yl)-3-(4-((3-chloro-1H-pyrrolo[2,3-b]pyridin-4-yl)oxy)cyclohexyl)urea

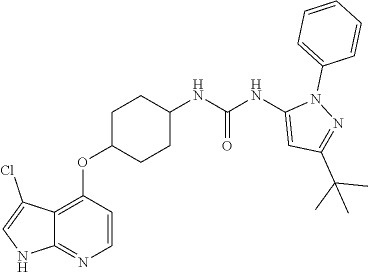 [SMILES: CC(C)(C)c1cc(NC(=O)NC2CCC(Oc3ccnc4[nH]cc(Cl)c34)CC2)n(-c2ccccc2)n1]

The title compound was obtained following the general procedure described for Example Ib-1, starting from 4-((3-chloro-1H-pyrrolo[2,3-b]pyridin-4-yl)oxy)cyclohexan-1-amine (Ib-D9, 0.035 g, 0.132 mmol) and phenyl (3-(tert-butyl)-1-phenyl-1H-pyrazol-5-yl)carbamate (Ib-E1, 0.044 g, 0.132 mmol), and was obtained as an off-white solid (5.9 mg, 9% yield). ¹H NMR (400 MHz, CD₃OD) δ=8.10 (s, 1H), 7.46-7.57 (m, 5H), 7.21 (s, 1H), 6.75 (d, J=6.0 Hz, 1H), 6.36 (s, 1H), 3.67-3.70 (m, 1H), 2.10-2.12 (m, 2H), 1.74-1.85 (m, 6H), 1.34 (s, 9H) LC-MS: 507.1 [M+H].

### Example IB-21

### 1-(3-(tert-butyl)-1-(1-methylpiperidin-4-yl)-1H-pyrazol-5-yl)-3-(4-((3-chloro-1H-pyrrolo[2,3-?]pyridin-4-yl)oxy)-2-fluorophenyl)urea

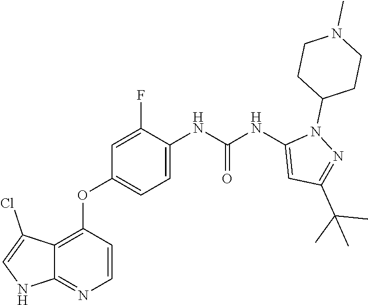 [SMILES: CN1CCC(n2nc(C(C)(C)C)cc2NC(=O)Nc2ccc(Oc3ccnc4[nH]cc(Cl)c34)cc2F)CC1]

The title compound was obtained following the general procedure described for Example Ib-1, starting from 4-((3-chloro-1H-pyrrolo[2,3-b]pyridin-4-yl)oxy)-2-fluoroaniline (Ib-D1, 0.078 g, 0.281 mmol) and phenyl (3-(tert-butyl)-1-(1-methylpiperidin-4-yl)-1H-pyrazol-5-yl)carbamate (Ib-414H, 0.100 g, 0.281 mmol), and was obtained as an off-white solid (4.5 mg, 3% yield). ¹H NMR (400 MHz, CD₃OD) δ=8.04-8.12 (m, 2H), 7.35 (s, 1H), 6.99-7.35 (m, 2H), 6.49 (d, J=5.60 Hz, 1H), 6.09 (s, 1H), 4.24-4.30 (m, 1H), 2.67-2.73 (m, 2H), 2.61 (s, 3H), 2.43-2.43 (m, 2H), 2.37-2.40 (m, 2H), 2.34 (s, 2H), 1.29 (s, 9H). LC-MS: 539.9 [M+H].

### Example IB-22

### N-(3-(tert-butyl)-1-phenyl-1H-pyrazol-5-yl)-2-(4-((3-chloro-1H-pyrrolo[2,3-b]pyridin-4-yl)oxy)-2-fluorophenyl)acetamide

### Step 1: Synthesis of N-(3-(tert-butyl)-1-phenyl-1H-pyrazol-5-yl)-2-(2-fluoro-4-hydroxyphenyl)acetamide

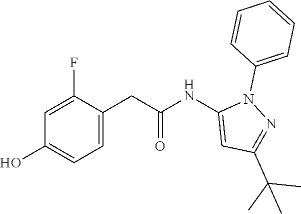 [SMILES: CC(C)(C)c1cc(NC(=O)Cc2ccc(O)cc2F)n(-c2ccccc2)n1]

T3P (50% in EtOAc, 1.132 g, 1.763 mmol) and DIPEA (0.791 mL, 4.410 mmol) were added to a solution of 2-(2-fluoro-4-hydroxyphenyl)acetic acid (0.250 g, 1.469 mmol) and 3-(tert-butyl)-1-phenyl-1H-pyrazol-5-anine (0.380 g, 1.763 mmol) in DCM (2 mL) and the resulting mixture was stirred at 25° C. for 12 h. Following completion of the reaction (as indicated by TLC), the reaction mixture was concentrated under reduced pressure to yield crude material which was purified by flash chromatography (silica gel 230-400 mesh, eluting with 20% EtOAc in petroleum ether), affording the title compound as a yellow gum (0.16 g, 28% yield) LC-MS: 368.1 [M+H].

### Step 2: Synthesis of N-(3-(tert-butyl)-1-phenyl-1H-pyrazol-5-yl)-2-(2-fluoro-4-((1-((2-(trimethylsilyl)ethoxy)methyl)-1H-pyrrolo[2,3-b]pyridin-4-yl)oxy)phenyl)acetamide

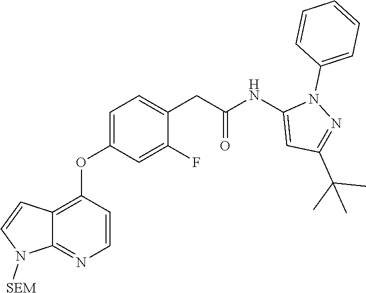 [SMILES: CC(C)(C)c1cc(NC(=O)Cc2ccc(Oc3ccnc4c3ccn4COCC[Si](C)(C)C)cc2F)n(-c2ccccc2)n1]

The title compound was obtained following a similar procedure described for Intermediate Ib-C1 (Method Ib-A), starting from 4-bromo-1-((2-(trimethylsilyl)ethoxy)methyl)-1H-pyrrolo[2,3-b]pyridine (Ib-A2, 0.100 g, 0.306 mmol), N-(3-(tert-butyl)-1-phenyl-1H-pyrazol-5-yl)-2-(2-fluoro-4-hydroxyphenyl)acetamide (0.119 g, 0.324 mmol), K₂CO₃ (0.093 g, 0.672 mmol), XPhos (0.022 g, 0.031 mmol) and Pd₂dba₃ (0.014 g, 0.015 mmol), and was obtained as a yellow gum (0.062 g, 29% yield). LC-MS: 614.3 [M+H].

### Step 3: Synthesis of N-(3-(tert-butyl)-1-phenyl-1H-pyrazol-5-yl)-2-(4-((3-chloro-1-((2-(trimethylsilyl)ethoxy)methyl)-1H-pyrrolo[2,3-b]pyridin-4-yl)oxy)-2-fluorophenyl)cetamide

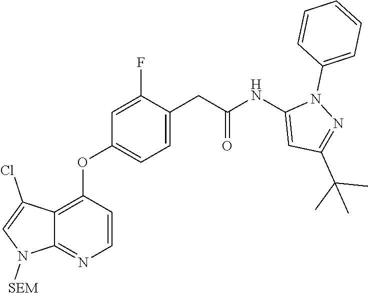 [SMILES: CC(C)(C)c1cc(NC(=O)Cc2ccc(Oc3ccnc4c3c(Cl)cn4COCC[Si](C)(C)C)cc2F)n(-c2ccccc2)n1]

The title compound was obtained following a similar procedure described for step 3 of Intermediate Ib-D8, starting from N-(3-(tert-butyl)-1-phenyl-1H-pyrazol-5-yl)-2-(2-fluoro-4-((1-((2-(trimethylsilyl)ethoxy)methyl)-1H-pyrrolo[2,3-b]pyridin-4-yl)oxy)phenyl)acetamide (0.040 g, 0.065 mmol) and NCS (0.008 g, 0.059 mmol), and was obtained as a yellow gum (0.04 g, 55% yield). LC-MS: 648.3 [M+H].

### Step 4: Synthesis of N-(3-(tert-butyl)-1-phenyl-1H-pyrazol-5-yl)-2-(4-((3-chloro-1H-pyrrolo[2,3-b]pyridin-4-yl)oxy)-2-fluorophenyl)acetamide

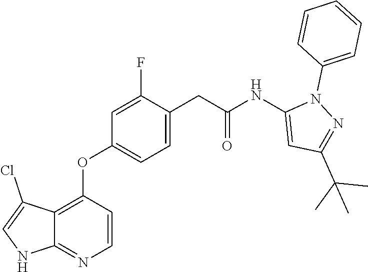 [SMILES: CC(C)(C)c1cc(NC(=O)Cc2ccc(Oc3ccnc4[nH]cc(Cl)c34)cc2F)n(-c2ccccc2)n1]

The title compound was obtained following a similar procedure described for Intermediate Ib-D, starting from N-(3-(tert-butyl)-1-phenyl-1H-pyrazol-5-yl)-2-(4-((3-chloro-1-((2-(trimethylsilyl)ethoxy)methyl)-1H-pyrrolo[2,3-b]pyridin-4-yl)oxy)-2-fluorophenyl)acetamide (0.040 g, 0.062 mmol), TFA (0.2 mL), and K₂CO₃ (0.026 g, 0.185 mmol), and was obtained as a brown gum which was further purified by RP-HPLC (using 10 mM NH₄OAC in water CAN), affording the title compound as an off-white solid (2.2 mg, 7% yield); ¹H NMR (400 MHz. CD₃OD) δ=8.13 (s, 1H), 7.32-7.47 (m, 7H), 6.95-7.00 (m, 2H), 6.62 (d, J=5.6 Hz, 1H), 6.36 (d, J=3.6 Hz, 1H), 3.76 (s, 2H), 1.45 (s, 9H). LC-MS: 518.2 [M+H].

### Intermediate IC-A

### 4-chloro-5-iodo-7H-pyrrolo[2,3-d]pyrimidine

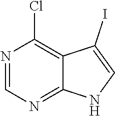 [SMILES: Clc1ncnc2[nH]cc(I)c12]

N-iodosuccinimide (1.465 g, 6.51 mmol) was added to a stirred solution of 4-chloro-7H-pyrrolo[2,3-d]pyrimidine (1.000 g, 6.51 mmol) in DMF (10 mL) at 0° C. and the resulting mixture was stirred at 25° C. for 12 h. Following completion of the reaction (as indicated by TLC), the reaction mixture was poured into ice cold water (100 mL) and stirred at 25° C. for 15 min. The resulting solid was filtered, washed with water (2×25 mL), and dried to afford the title compound as an off-white solid (1.7 g, 93% yield) ¹H NMR (400 MHz, DMSO-d₆) δ=12.96 (bs, 1H), 8.60 (s, 1H), 7.95 (d, J=2.40 Hz, 1H); LCMS: 279.9 [M+H].

### Intermediate IC-B1

### 4-chloro-7-cyclopropyl-5-iodo-7H-pyrrolo[2,3-d]pyrimidine

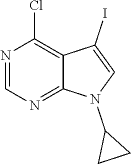 [SMILES: Clc1ncnc2c1c(I)cn2C1CC1]

Copper (II) acetate (0.650 g, 3.58 mmol), 2,2′-bipyridine (0.559 g, 3.58 mmol), and sodium bicarbonate (0.601 g, 7.16 mmol) were added to a solution of 4-chloro-5-iodo-7H-pyrrolo[2,3-d]pyrimidine (Ic-A, 1.000 g, 3.58 mmol) and cyclopropylboronic acid (0.615 g, 7.16 mmol) in dichloroethane (10 mL) and the resulting mixture was stirred at 70° C. under oxygen atmosphere for 12 h. Following completion of the reaction (as indicated by TLC), the reaction mixture was filtered through a pad of of diatomaceous earth which was then rinsed with DCM (2.20 mL). The combined filtrates were washed with water (20 mL) and brine (25 mL), dried over Na₂SO₄, filtered, and concentrated under reduced pressure to give crude material which was purified by flash chromatography (silica gel 230-400 mesh, eluting with 15% EtOAc in petroleum ether), affording the title compound as an off-white solid (0.7 g, 61% yield). ¹H NMR (400 MHz, DMSO-d₆) δ=8.67 (s, 1H), 7.96 (s, 1H), 3.63-3.69 (m, 1H), 1.06-1.10 (m, 4H). LCMS: 319.9 [M+H].

### Intermediate IC-B2

### 4-chloro-5-iodo-7-(oxetan-3-yl)-7H-pyrrolo[2,3-d]pyridine

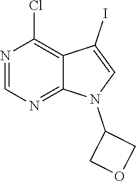 [SMILES: Clc1ncnc2c1c(I)cn2C1COC1]

K₂CO₃ (0.40 g, 2.86 mmol) and 3-iodooxetane (0.32 g, 1.71 mmol) were added to a solution of 4-chloro-5-iodo-7H-pyrrolo[2,3-d]pyrimidine (Ic-A, 0.40 g, 1.43 mmol) in DMF (5 mL) and the resulting mixture was stirred at 90° C. for 16 h in a sealed tube. Following completion of the reaction (as indicated by TLC), the reaction mixture was poured into crushed ice (50 g) and stirred for 15 min. The resulting solid was filtered, washed with water (2×5 mL), and dried to afford the title compound as an off-white solid (0.2 g, 42% yield). LCMS: 335.7 [M+H].

### Intermediate IC-B3

### 1-(4-chloro-5-iodo-7H-pyrrolo[2,3-d]pyrimidin-7-yl)-2-methylpropan-2-ol

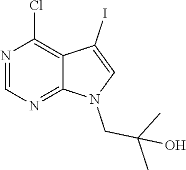 [SMILES: CC(C)(O)Cn1cc(I)c2c(Cl)ncnc21]

NaH₂PO₄ (0.105 g, 0.877 mmol) was added to a mixture of 4-chloro-5-iodo-7H-pyrrolo[2,3-d]pyrimidine (Ic-A, 0.250 g, 0.895 mmol), 2,2-dimethyloxirane (0.157 ml, 1.762 mmol), and K₂CO₃ (0.121 g, 0.877 mmol) in ACN (3 mL) and water (1 mL). The resulting mixture was subjected to microwave irradiation at 150° C. for 1 h in a sealed tube. Following completion of the reaction (as indicated by TLC), the reaction mixture was concentrated under reduced pressure and the resulting crude material was purified by flash chromatography (silica gel 230-400 mesh, eluting with 18% EtOAc in petroleum ether), affording the title compound as a pale brown solid (0.1 g, 17% yield). LCMS: 351.9 [M+H].

### Intermediate IC-B4

### 4-chloro-5-iodo-7-(pyridin-3-yl)-7H-pyrrolo[2,3-d]pyrimidine

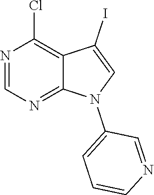 [SMILES: Clc1ncnc2c1c(I)cn2-c1cccnc1]

Triethylamine (0.905 g, 8.95 mmol) and copper (II) acetate (0.975 g, 5.37 mmol) were added to a solution of 4-chloro-5-iodo-7H-pyrrolo[2,3-d]pyrimidine (Ic-A, 1.000 g, 3.58 mmol) and 3-pyridinylboronic acid (0.880 g, 7.16 mmol) in DCM (25 mL) and the resulting mixture was stirred at 40° C. under oxygen atmosphere for 40 h. Following completion of the reaction (as indicated by LCMS), the reaction mixture was filtered through a pad of of diatomaceous earth which was then rinsed with DCM (2×50 mL). The combined filtrates were washed with water (10 mL) and brine (10 mL), dried over Na₂SO₄, filtered, and concentrated under reduced pressure to give crude material. This was stirred with 30% diethyl ether in petroleum ether for 30 minutes at 25° C., filtered, and dried to afford the title compound as a brown solid (0.4 g, 29% yield). ¹H NMR (400 MHz, DMSO-d₆) δ=9.05 (bs, 1H), 8.73 (s, 1H), 8.67 (bs, 1H), 8.48 (s, 1H), 8.26-8.28 (m, 1H), 7.64-7.67 (m, 1H). LCMS: 356.8 [M+H].

### Intermediate IC-B5

### 4-chloro-5-iodo-7-(pyridin-4-yl)-7H-pyrrolo[2,3-b]pyrimidine

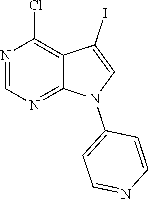 [SMILES: Clc1ncnc2c1c(I)cn2-c1ccncc1]

The title compound was obtained by following a similar procedure described for Ic-B4, starting from 4-chloro-5-iodo-7H-pyrrolo[2,3-d]pyrimidine (Ic-A, 0.50 g, 1.789 mmol) and 4-pyridinylboronic acid (0.44 g, 3.580 mmol), and was obtained as a brown solid (0.21 g, 29% yield). LCMS: 356.9 [M+H].

### Intermediate IC-B6

### 4-chloro-5-iodo-7-(1-methylpiperidin-4-yl)-7H-pyrrolo([2,3-d]pyrimidine

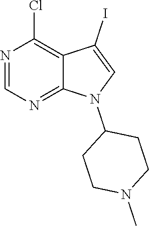 [SMILES: CN1CCC(n2cc(I)c3c(Cl)ncnc32)CC1]

The title compound was prepared as reported in PCT Pub. No. WO 2017/220477.

### Intermediate IC-B7

### 7-(3-(Benzyloxy)cyclobutyl)-A-chloro-5-iodo-7H-pyrrolo[2,3-D]pyrimidine

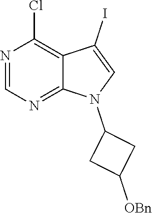 [SMILES: Clc1ncnc2c1c(I)cn2C1CC(OCc2ccccc2)C1]

Cs₂CO₃ (0.583 g, 1.789 mmol) and 3-(benzyloxy)cyclobutyl methanesulfonate (prepared as reported in PCT Pub No. WO 2019/092170, 0.459 g, 1.789 mmol) were added to a solution of 4-chloro-5-iodo-7/1-pyrrolo[2,3-d]pyrimidine (Ic-A, 0.250 g, 0.895 mmol) in DMF (5 mL) and the resulting mixture was stirred at 90° C. for 12 h. Following completion of the reaction (as indicated by TLC), the reaction mixture was poured into ice water (50 mL) and extracted with ethyl acetate (2×30 mL). The combined organic extracts were dried over Na₂SO₄, filtered, and concentrated under reduced pressure to give crude material which was purified by flash chromatography (silica gel 230-400 mesh, eluting with 30% EtOAc in petroleum ether), affording the title compound as a colorless gum (0.14 g, 31% yield). LCMS: 440.0 [M+H].

### Intermediate IC-B8

### 2-(4-chloro-5-iodo-7H-pyrrolo[2,3-d]pyrimidin-7-yl)ethan-1-ol

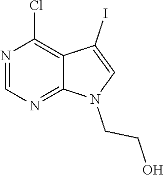 [SMILES: OCCn1cc(I)c2c(Cl)ncnc21]

K₂CO₃ (0.742 g, 5.37 mmol) and 2-bromoethan-1-ol (0.537 g, 4.29 mmol) were added to a solution of 4-chloro-5-iodo-7H-pyrrolo[2,3-d]pyrimidine (Ic-A1.000 g, 3.58 mmol) in DMF (6 mL) and the resulting suspension was stirred at 80° C. for 2 h. Following completion of the reaction (as indicated by TLC), the reaction mixture was poured into crushed ice (25 g). The resulting solid was filtered, washed with water (20 mL), and dried to afford the title compound as a yellow solid (0.84 g, 64% yield). LCMS: 323.9 [M+H].

### Intermediate IC-B9

### 4-chloro-1-cyclopropyl-3-iodo-1H-pyrrolo[3,2-c]pyridine

### Step 1: Synthesis of 4-chloro-1-cyclopropyl-1H-pyrrolo[3,2-c]pyridine

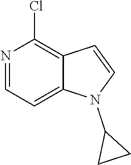 [SMILES: Clc1nccc2c1ccn2C1CC1]

Triethylamine (0.332 g, 3.280 mmol), copper (II) acetate (0.298 g, 1.638 mmol), and molecular sieves (powdered, 0.050 g) were added to a solution of 4-chloro-1H-pyrrolo[3,2-c]pyridine (0.250 g, 1.638 mmol) and cyclopropyl boronic acid (0.279 g, 3.280 mmol) in DMF (10 mL) and the resulting suspension was stirred at 60° C. for 12 h in a sealed tube. Following completion of the reaction (as indicated by TLC), the reaction mixture was filtered through a pad of of diatomaceous earth which was then rinsed with EtOAc. The combined filtrates were concentrated under reduced pressure, giving crude material which was purified by Isolera (silica gel 230-400 mesh, eluting with 20% EtOAc in petroleum ether), affording the title compound as a yellow solid (0.19 g, 59% yield). ¹H NMR (400 MHz, DMSO-d₆) δ=8.04 (d, J=5.6 Hz, 1H), 7.56-7.60 (m, 2H), 6.52-6.53 (m, 1H), 3.55-3.58 (m, 1H), 1.00-1.13 (m, 4H). LCMS: 193.1 [M+H].

### Step 2: Synthesis of 4-chloro-1-cyclopropyl-3-iodo-1H-pyrrolo[3,2-c]pyridine

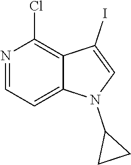 [SMILES: Clc1nccc2c1c(I)cn2C1CC1]

N-iodosuccinimide (0.350 g, 1.557 mmol) was added to a solution of 4-chloro-1-cyclopropyl-1H-pyrrolo[3,2-c]pyridine (0.200 g, 1.038 mmol) in DMF (5 mL) and the resulting mixture was stirred at 80° C. for an hour. Following completion of the reaction (as indicated by LCMS), the reaction mixture was poured into crushed ice (25 g) and extracted with EtOAc (2×25 mL). The combined organic extracts were washed with brine, dried over Na₂SO₄, filtered, and concentrated under reduced pressure to yield the title product (0.2 g) which was used without further purification. LCMS: 319.0 [M+H].

### Intermediate IC-C1

### 7-cyclopropyl-5-iodo-7H-pyrrolo[2,3-d]pyrimidin-4-amine

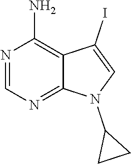 [SMILES: Nc1ncnc2c1c(I)cn2C1CC1]

A mixture of 4-chloro-7-cyclopropyl-5-iodo-7/1-pyrrolo[2,3-d]pyrimidine (Ic-B1, 1.00 g, 2.191 mmol) and ammonium hydroxide (25% in water, 5 mL) was subjected to microwave irradiation at 150° C. for 1 h. Following completion of the reaction (as indicated by TLC), the reaction mixture was concentrated under reduced pressure to afford the title compound as an off-white solid (0.75 g, 80% yield). ¹H NMR (400 MHz, DMSO-d₆) δ=8.12 (s, 1H), 7.39 (s, 1H), 6.57 (bs, 2H), 3.48-3.54 (m, 1H), 0.97-1.01 (m, 4H). LCMS: 301.0 [M+H].

### Intermediate IC-C2

### 5-iodo-7-(oxetan-3-yl)-7H-pyrrolo[2,3-d]pyrimidin-4-amine

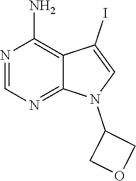 [SMILES: Nc1ncnc2c1c(I)cn2C1COC1]

The title compound was prepared via a similar procedure described for Ic-C1, starting from 4-chloro-5-iodo-7-(oxetan-3-yl)-7H-pyrrolo[2,3-d]pyrimidine (Ic-B2, 0.5 g, 1.49 mmol) and aqueous ammonium hydroxide (25% in water, 2.5 mL), and was obtained as a pale brown solid (0.27 g, 58% yield) LCMS: 316.8 [M+H].

### Intermediate IC-C3

### 1-(4-amino-5-iodo-7H-pyrrolo[2,3-d]pyrimidin-7-yl)-2-methylpropan-2-ol

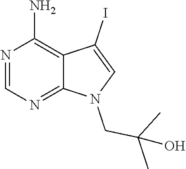 [SMILES: CC(C)(O)Cn1cc(I)c2c(N)ncnc21]

The title compound was obtained by following a similar procedure described for Ic-C1, starting from 1-(4-chloro-5-iodo-71H-pyrrolo[2,3-d]pyrimidin-7-yl)-2-methylpropan-2-ol (Ic-B3, 0.1 g, 0.284 mmol) and ammonium hydroxide (25% in water, 0.5 mL), and was obtained as an off-white solid (0.08 g, 85% yield). ¹H NMR (400 MHz, DMSO-d₆) δ=8.12 (s, 1H), 7.38 (s, 1H), 4.81 (s, 1H), 4.04 (s, 2H), 1.03 (s, 6H). LCMS: 333.0 [M+H].

### Intermediate IC-C4

### 5-iodo-7-(pyridin-3-yl)-7H-pyrrolo[2,3-d]pyrimidin-4-amine

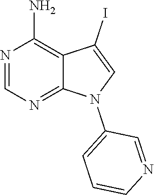 [SMILES: Nc1ncnc2c1c(I)cn2-c1cccnc1]

Ammonium hydroxide (25% in water, 1 mL) was added to a solution of 4-chloro-5-iodo-7-(pyridin-3-yl)-7H-pyrrolo[2,3-d]pyrimidine (Ic-B4, 0.30 g, 0.841 mmol) in dioxane (10 mL) and the resulting mixture was subjected to microwave irradiation at 150° C. for 2 h. Following completion of the reaction (as indicated by LCMS), the reaction mixture was concentrated under reduced pressure to yield crude material which was washed with methyl tert-butyl ether and dried, affording the title compound as an off-white solid (0.21 g, 63% yield). LCMS: 337.8 [M+H].

### Intermediate IC-C5

### 5-iodo-(pyridin-4-yl)-7H-pyrrolo[2,3-d]pyrimidin-4-amine

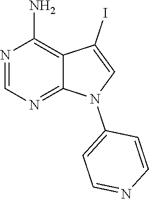 [SMILES: Nc1ncnc2c1c(I)cn2-c1ccncc1]

The title compound was obtained by following a similar procedure described for Ic-C4, starting from 4-chloro-5-iodo-7-(pyridin-4-yl)-7H-pyrrolo[2,3-d]pyrimidine (Ic-B5, 0.21 g, 0.589 mmol) and ammonium hydroxide (25% in water, 1 mL), and was obtained as an off-white solid (0.16 g, 69% yield). LCMS: 337.9 [M+H].

### Intermediate IC-C6

### 5-iodo-7-(1-methylpiperidin-4-yl)-7H-pyrrolo[2,3-d]pyrimidin-4-amine

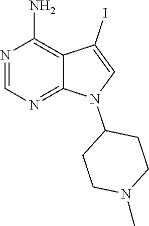 [SMILES: CN1CCC(n2cc(I)c3c(N)ncnc32)CC1]

The title compound was prepared as reported in PCT Pub. No. WO 2017/220477.

### Intermediate IC-C7

### 7-(3-(benzyloxy)cyclobutyl)-5-iodo-7H-pyrrolo[2,3-d]pyrimidin-4-amine

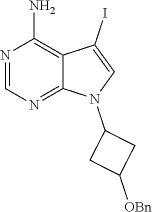 [SMILES: Nc1ncnc2c1c(I)cn2C1CC(OCc2ccccc2)C1]

The title compound was obtained by following a similar procedure described for Ic-C₄, starting from 7-(3-(benzyloxy)cyclobutyl)-4-chloro-5-iodo-7H-pyrrolo[2,3-d]pyrimidine (Ic-B7, 0.140 g, 0.318 mmol) and ammonium hydroxide (25% in water, 1.4 mL), and was obtained as an off-white solid (0.06 g, 45% yield). LCMS: 421.1 [M+H].

### Intermediate IC-C8

### 5-iodo-7-(2-methoxyethyl)-7H-pyrrolo[2,3-d]pyrimidin-4-amine

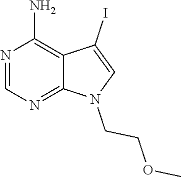

The title compound was prepared as reported in PCT Pub. No. WO 2014/184069A1.

### Intermediate IC-C9

### 2-(4-amino-5-iodo-7H-pyrrolo[2,3-d]pyrimidin-7-yl)ethan-1-ol

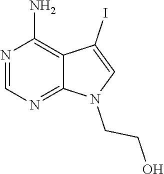 [SMILES: Nc1ncnc2c1c(I)cn2CCO]

The title compound was obtained by following a similar procedure described for Ic-C4, starting from 2-(4-chloro-5-iodo-7H-pyrrolo[2,3-d]pyrimidin-7-yl)ethan-1-ol (Ic-B8, 0.84 g, 2.61 mmol) and ammonium hydroxide (25% in water, 8 mL), and was obtained as an off-white solid (0.94 g, 69% yield). LCMS: 305.0 [M+H].

### Intermediate IC-C10

### 7-cyclobutyl-5-iodo-7H-pyrrolo[2,3-d]pyrimidin-4-amine

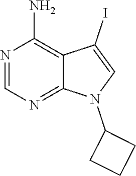 [SMILES: Nc1ncnc2c1c(I)cn2C1CCC1]

The title compound was prepared as reported in PCT Pub. No. WO 2016/075224

### Intermediate IC-C11

### 5-iodo-7-(1-methylpyrrolidin-3-yl)-7H-pyrrolo[2,3-d]pyrimidin-4-amine

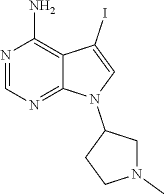 [SMILES: CN1CCC(n2cc(I)c3c(N)ncnc32)C1]

The title compound was prepared as reported in PCT Pub. No. WO 2016/075224.

### Intermediate IC-D12

### 5-(4-amino-3-fluorophenyl)-7-cyclopropyl-7H-pyrrolo[2,3-d]pyrimidin-4-amine

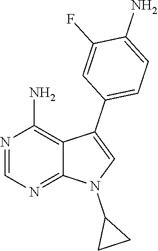 [SMILES: Nc1ccc(-c2cn(C3CC3)c3ncnc(N)c23)cc1F]

A mixture of 7-cyclopropyl-5-iodo-7H-pyrrolo[2,3-d]pyrimidin-4-amine (Ic-C1, 0.160 g, 0.533 mmol), 2-fluoro-4-(4,4,5,5-tetramethyl-1,3,2-dioxaborolan-2-yl)aniline (0.190 g, 0.800 mmol), and K₂CO₃ (0.221 g, 1.599 mmol) in 1,4-dioxane (1 mL) and water (0.3 mL) was purged with N₂ for 10 min. Pd(PPh₃)₄ (0.062 g, 0.053 mmol) was then added and the reaction mixture was stirred at 100° C. for 12 h. Following completion of the reaction (as indicated by TLC), the mixture was filtered through a pad of diatomaceous earth which was then rinsed with EtOAc (2.10 mL). The combined filtrates were concentrated under reduced pressure to yield crude material which was purified by flash chromatography (silica gel 230-400 mesh, eluting with 3% MeOH in DCM), affording the title compound as a yellow solid (0.110 g, 73% yield). ¹H NMR (400 MHz, DMSO-d₆) δ=8.14 (s, 1H), 7.13 (s, 1H), 7.05-7.09 (m, 1H), 6.95-6.98 (m, 1H), 6.82-6.86 (m, 1H), 6.10 (bs, 2H), 5.22 (bs, 2H), 3.52-3.58 (m, 1H), 1.00-1.04 (m, 4H). LCMS: 284.1 [M+H].

### Intermediate IC-D2

### 5-(4-aminophenyl)-7-(oxetan-3-yl)-7H-pyrrolo[2,3-d]pyrimidin-4-amine

### Step 1: Synthesis of 5-(4-nitrophenyl)-7-(oxetan-3-yl)-7H-pyrrolo[2,3-d]pyrimidin-4-amine

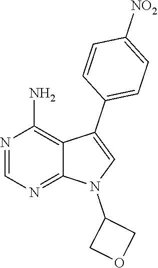 [SMILES: Nc1ncnc2c1c(-c1ccc([N+](=O)[O-])cc1)cn2C1COC1]

The title compound was prepared via a similar procedure described for Ic-D1, starting from 4-chloro-5-iodo-7-(oxetan-3-yl)-7H-pyrrolo[2,3-d]pyrimidine (Ic-C2, 0.252 g, 0.797 mmol) and (4-nitrophenyl)boronic acid (0.200 g, 1.198 mmol), and was obtained as a pale brown solid (0.143 g, 58% yield). LCMS: 312.1 [M+H].

### Step 2: Synthesis of 5-(4-aminophenyl)-7-(oxetan-3-yl)-?H-pyrrolo[2,3-d]pyrimidin-4-amine

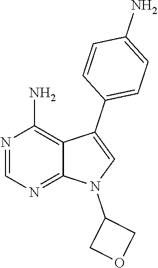 [SMILES: Nc1ccc(-c2cn(C3COC3)c3ncnc(N)c23)cc1]

Iron powder (0.251 g, 4.5 mmol) and ammonium chloride (0.240 g, 4.5 mmol) were added to a solution of 5-(4-nitrophenyl)-7-(oxetan-3-yl)-7H-pyrrolo[2,3-d]pyrimidin-4-amine (0.14 g, 0.45 mmol) in ethanol (5 mL) and water (2 mL) and the resulting mixture was stirred at 80° C. for 3 h. Following completion of the reaction (as indicated by TLC), the reaction mixture was filtered through a pad of of diatomaceous earth which was then rinsed with EtOAc (2×5 mL). The combined filtrates were concentrated under reduced pressure, giving a residue which was dissolved in EtOAc (25 mL), washed with brine (5 mL), dried over Na₂SO₄, filtered, and concentrated under reduced pressure to afford the title compound as a brown solid (0.12 g, quantitative yield) which was used without further purification. LCMS: 281.9 [M+H].

### Intermediate IC-D3

### 1-(4-amino-5-(4-amino-3-fluorophenyl)-7H-pyrrolo[2,3-d]pyrimidin-7-yl)-2-methylpropan-2-ol

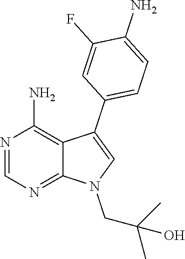 [SMILES: CC(C)(O)Cn1cc(-c2ccc(N)c(F)c2)c2c(N)ncnc21]

The title compound was prepared via a similar procedure described for Ic-D1, starting from 1-(4-amino-5-iodo-7H-pyrrolo[2,3-d]pyrimidin-7-yl)-2-methylpropan-2-ol (Ic-C₃ (0.100 g, 0.301 mmol) and 2-fluoro-4-(4,4,5,5-tetramethyl-1,3,2-dioxaborolan-2-yl)aniline (0.086 g, 0.361 mmol), and was obtained as a pale yellow gum (0.05 g, 53% yield). LCMS: 316.1 [M+H].

### Intermediate IC-D4

### 5-(4-aminophenyl)-7-cyclopropyl-7H-pyrrolo[2,3-d]pyrimidin-4-amine

### Step 1: Synthesis of 7-cyclopropyl-5-(4-nitrophenyl)-7H-pyrrolo[2,3-d]pyrimidin-4-amine

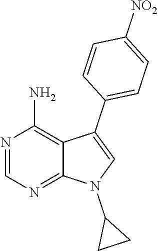 [SMILES: Nc1ncnc2c1c(-c1ccc([N+](=O)[O-])cc1)cn2C1CC1]

The title compound was obtained by following a similar procedure described for Ic-D1, starting from 7-cyclopropyl-5-iodo-7H-pyrrolo[2,3-d]pyrimidin-4-amine (Ic-C1, 0.18 g, 0.60 mmol) and (4-nitrophenyl)boronic acid (0.12 g, 0.72 mmol), and was obtained as a pale brown solid (0.10 g, 56% yield). ¹H NMR (400 MHz, DMSO-d₆)=8.28-8.32 (m, 2H), 8.21 (s, 1H), 7.77 (s, 1H), 7.70-7.73 (m, 2H), 7.55 (s, 1H), 5.69 (bs, 2H), 3.61-3.64 (m, 1H), 1.04-1.09 (m, 4H). LCMS: 296.1 [M+H].

### Step 2: Synthesis of 5-(4-nitrophenyl)-7-cyclopropyl-7H-pyrrolo[2,3-d]pyrimidin-1-amine

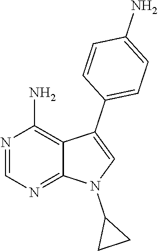 [SMILES: Nc1ccc(-c2cn(C3CC3)c3ncnc(N)c23)cc1]

The title compound was obtained by following a similar procedure described for step 2 of Ic-D2, starting from 7-cyclopropyl-5-(4-nitrophenyl)-7H-pyrrolo[2,3-d]pyrimidin-4-amine (0.10 g, 0.33 mmol) and Fe/NH₄Cl, and was obtained as a brown gum (0.08 g, 90% yield) which was used without further purification. ¹H NMR (400 MHz, DMSO-d₆) δ=8.13 (s, 1H), 7.04-7.11 (m, 2H), 7.04 (s, 1H), 6.63-6.67 (m, 2H), 6.05 (bs, 2H), 5.27 (bs, 2H), 3.51-3.57 (m, 1H), 0.99-1.04 (m, 4H) LCMS: 266.0 [M+H].

### Intermediate IC-D5

### 5-(4-amino-2-fluorophenyl)-7-cyclopropyl-7H-pyrrolo[2,3-d]pyrimidin-4-amine

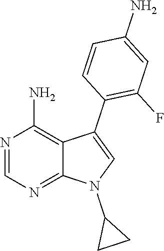 [SMILES: Nc1ccc(-c2cn(C3CC3)c3ncnc(N)c23)c(F)c1]

The title compound was obtained by following a similar procedure described for Ic-D1, starting from 7-cyclopropyl-5-iodo-7H-pyrrolo[2,3-d]pyrimidin-4-amine (Ic-C1, 0.21 g, 0.69 mmol) and 3-fluoro-4-(4,4,5,5-tetramethyl-1,3,2-dioxaborolan-2-yl)aniline (0.20 g, 0.83 mmol), and was obtained as a pale yellow gum (0.15 g, 76% yield) LCMS: 284.1 [M+H].

### Intermediate IC-D6

### 5-(6-aminopyridin-3-yl)-7-cyclopropyl-7H-pyrrolo[2,3-d]pyrimidin-4-amine

### Step 1: Synthesis of 7-cyclopropyl-5-(6-nitropyridin-3-yl)-?H-pyrrolo[2,3-d]pyrimidin-4-amine

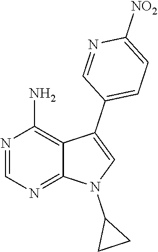 [SMILES: Nc1ncnc2c1c(-c1ccc([N+](=O)[O-])nc1)cn2C1CC1]

The title compound was obtained by following a similar procedure described for Ic-D1, starting from 7-cyclopropyl-5-iodo-7H-pyrrolo[2,3-d]pyrimidin-4-amine (Ic-C1, 0.28 g, 0.94 mmol) and 2-nitro-5-(4,4,5,5-tetramethyl-1,3,2-dioxaborolan-2-yl)pyridine (0.28 g, 1.13 mmol), and was obtained as a pale brown solid (0.16 g, 57% yield). ¹H NMR (400 MHz, DMSO-d₆) δ=8.73 (d, J=2.0 Hz, 1H), 8.39 (d, J=8.8 Hz, 1H), 8.23 (s, 1H), 8.17-8.20 (m, 1H), 7.67 (s, 1H), 6.49 (bs, 2H), 3.61-3.67 (m, 1H), 1.06-1.09 (m, 41). LCMS: 297.1 [M+H].

### Step 2: Synthesis of 5-(6-aminopyridin-3-yl)-7-cyclopropyl-7H-pyrrolo[2,3-d]pyrimidin-5-yl)cyclohex-3-en-1-yl)carbamate

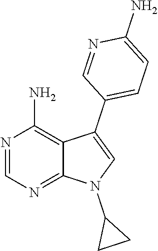 [SMILES: Nc1ccc(-c2cn(C3CC3)c3ncnc(N)c23)cn1]

The title compound was obtained by following a similar procedure described for step 2 of Ic-D2, starting from 7-cyclopropyl-5-(6-nitropyridin-3-yl)-7H-pyrrolo[2,3-d]pyrimidin-4-amine (0.16 g, 0.54 mmol) and Fe/NH₄Cl, and was obtained as a pale brown solid (0.1 g, 70% yield) which was used without further purification. LCMS: 267.0 [M+H].

### Intermediate IC-D7

### 5-(4-aminocyclohex-1-en-1-yl)-7-cyclopropyl-7H-pyrrolo[2,3-d]pyrimidin-4-amine

### Step 1: synthesis of tert-butyl (4-(4-amino-?-cyclopropyl-7H-pyrrolo[2,3-d]pyrimidin-5-yl)cyclohex-3-en-1-yl)carbamate

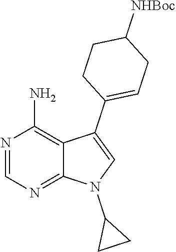 [SMILES: CC(C)(C)OC(=O)NC1CC=C(c2cn(C3CC3)c3ncnc(N)c23)CC1]

K₂CO₃ (0.318 g, 2.299 mmol) was added to a solution of 7-cyclopropyl-5-iodo-711-pyrrolo[2,3-d]pyrimidin-4-amine (Ic-C1, 0.230 g, 0.766 mmol) and tert-butyl (4-(4,4,5,5-tetramethyl-1,3,2-dioxaborolan-2-yl)cyclohex-3-en-1-yl)carbamate (0.372 g, 1.150 mmol) in dioxane (1 mL) and water (0.3 mL). The solution was purged with N₂ for 10 min then Pd(PPh₃)₄ (0.044 g, 0.038 mmol) was added and the resulting mixture was subjected to microwave irradiation at 100° C. for 1 h. Following completion of the reaction (as indicated by TLC), the reaction mixture was filtered through a pad of of diatomaceous earth which was then rinsed with EtOAc (2×10 mL). The combined filtrates were concentrated under reduced pressure, giving crude material which was purified by preparative HPLC (mass-based, eluting with a gradient of ammonium acetate in water and ACN) to afford the title product as a pale-yellow gum (0.18 g, 62% yield). LCMS: 370.2 [M+H].

### Step 2: Synthesis of 5-(4-aminocyclohex-1-en-1-yl)-7-cyclopropyl-7H-pyrrolo[2,3-d]pyrimidin-4-amine

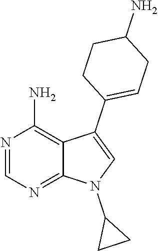 [SMILES: Nc1ncnc2c1c(C1=CCC(N)CC1)cn2C1CC1]

TFA (0.012 g, 0.108 mmol) was added to a solution of tert-butyl (4-(4-amino-7-cyclopropyl-7H-pyrrolo[2,3-d]pyrimidin-5-yl)cyclohex-3-en-1-yl)carbamate (0.040 g, 0.108 mmol) in DCM (2 mL) at 0° C. and the resulting solution was stirred at room temperature for 12 h. Following completion of the reaction (as indicated by TLC), the reaction mixture was concentrated under reduced pressure to afford the title product as a brown gum (0.029 g) which was used without further purification. LCMS: 270.1[M+H].

### Intermediate IC-D8

### 5-(4-amino-3-fluorophenyl)-7-(pyridin-3-yl)-7H-pyrrolo[2,3-d]pyrimidin-4-amine

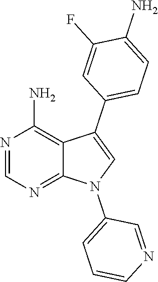 [SMILES: Nc1ccc(-c2cn(-c3cccnc3)c3ncnc(N)c23)cc1F]

A mixture of 5-iodo-7-(pyridin-3-yl)-7H-pyrrolo[2,3-d]pyrimidin-4-amine (Ic-C4, 0.160 g, 0.475 mmol), 2-fluoro-4-(4,4,5,5-tetramethyl-1,3,2-dioxaborolan-2-yl)aniline (0.169 g, 0.712 mmol), and K₂CO₃ (0.131 g, 0.949 mmol) in dioxane (5 mL), water (2 mL), and ethanol (3 mL) was purged with N₂ for 10 minutes. PdCl₂(dppf) (0.017 g, 0.024 mmol) was added and the resulting mixture was subjected to microwave irradiation at 100° C. for 1 h. Following completion of the reaction (as indicated by LCMS), the reaction mixture was filtered through a pad of of diatomaceous earth which was then rinsed with EtOAc (5 mL). The combined filtrates were concentrated under reduced pressure to give a residue which was taken in EtOAc (50 mL), washed with water (5 mL) and brine (5 mL), dried over Na₂SO₄, filtered and concentrated under reduced pressure. The resulting crude material was purified by GRACE (silica gel 230-400 mesh, eluting with 4% MeOH in DCM) to afford the title compound as a brown solid (0.2 g, 70% yield). LCMS: 321.0 [M+H].

### Intermediate IC-D9

### 5-(4-amino-3-fluorophenyl)-7-(pyridin-4-yl)-7H-pyrrolo[2,3-b]pyrimidin-4-amine

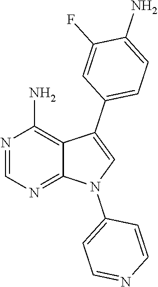 [SMILES: Nc1ccc(-c2cn(-c3ccncc3)c3ncnc(N)c23)cc1F]

The title compound was obtained by following a similar procedure described for Ic-D8, starting from 5-iodo-7-(pyridin-4-yl)-7H-pyrrolo[2,3-d]pyrimidin-4-amine (Ic-C5, 0.160 g, 0.475 mmol) and 2-fluoro-4-(4,4,5,5-tetramethyl-1,3,2-dioxaborolan-2-yl)aniline (0.169 g, 0.712 mmol), and was obtained as a brown solid (0.08 g, 51% yield). LCMS: 321.0 [M+H].

### Intermediate IC-D10

### 5-(4-amino-3-fluorophenyl)-7-(1-methylpiperidin-4-yl)-7H-pyrrolo[2,3-d]pyrimidin-4-amine

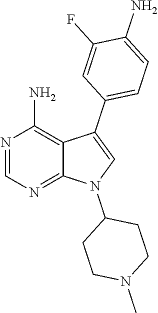 [SMILES: CN1CCC(n2cc(-c3ccc(N)c(F)c3)c3c(N)ncnc32)CC1]

The title compound was obtained by following a similar procedure described for Ic-D8, starting from 5-iodo-7-(1-methylpiperidin-4-yl)-7H-pyrrolo[2,3-d]pyrimidin-4-amine (Ic-C6, 0.180 g, 0.504 mmol) and 2-fluoro-4-(4,4,5,5-tetramethyl-1,3,2-dioxaborolan-2-yl)aniline (0.131 g, 0.554 mmol), and was obtained as a brown gum (0.15 g, 80% yield) LCMS: 341.1 [M+H].

### Intermediate IC-D1

### 5-(4-amino-3-methylphenyl)-7-cyclopropyl-7H-pyrrolo[2,3-d]pyrimidin-4-amine

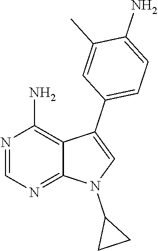 [SMILES: Cc1cc(-c2cn(C3CC3)c3ncnc(N)c23)ccc1N]

The title compound was obtained by following a similar procedure described for Ic-D8, starting from 7-cyclopropyl-5-iodo-7H-pyrrolo[2,3-d]pyrimidin-4-amine (Ic-C1, 0.250 g, 0.833 mmol) and 2-methyl-4-(4,4,5,5-tetramethyl-1,3,2-dioxaborolan-2-yl)aniline (0.214 g, 0.916 mmol), and was obtained as a brown gum (0.13 g, 56% yield). LCMS: 280.1 [M+H].

### Intermediate IC-D12

### 5-(4-amino-3-fluorophenyl)-7-(3-(benzyloxy)cyclobutyl)-7H-pyrrolo[2,3-d]pyrimidin-4-amine

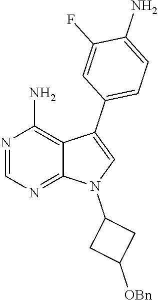 [SMILES: Nc1ccc(-c2cn(C3CC(OCc4ccccc4)C3)c3ncnc(N)c23)cc1F]

The title compound was obtained by following a similar procedure described for Ic-D8, starting from 7-(3-(benzyloxy)cyclobutyl)-5-iodo-7H-pyrrolo[2,3-d]pyrimidin-4-amine (Ic-C7, 0.060 g, 0.143 mmol) and 2-fluoro-4-(4,4,5,5-tetramethyl-1,3,2-dioxaborolan-2-yl)aniline (0.037 g, 0.157 mmol), and was obtained as a brown solid (0.03 g, 53% yield). LCMS: 404.2 [M+H].

### Intermediate IC-D13

### Step 1: Synthesis of 7-cyclopropyl-5-(4,4,5,5-tetramethyl-1,3,2-dioxaborolan-2-yl)-7H-pyrrolo[2,3-d]pyrimidin-4-amine

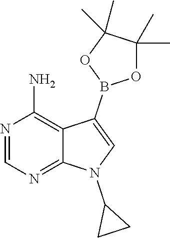 [SMILES: CC1(C)OB(c2cn(C3CC3)c3ncnc(N)c23)OC1(C)C]

Potassium acetate (0.245 g, 2.499 mmol) was added to a solution of 7-cyclopropyl-5-iodo-7H-pyrrolo[2,3-d]pyrimidin-4-amine (Ic-C1, 0.250 g, 0.833 mmol) and bis(pinacolato)diboron (0.317 g, 1.250 mmol) in DMSO (5 mL) and the resulting mixture was purged with N₂ for 10 min. PdCl₂(dppf) (0.030 g, 0.042 mmol) was then added and the reaction mixture was stirred at 85° C. for 2 h. Following completion of the reaction, the reaction mixture was filtered through a pad of of diatomaceous earth which was then rinsed with DCM (2×20 mL). The combined filtrates were concentrated under reduced pressure to yield the title compound as a black residue which was used without further purification. LCMS: 300.9 [M+H].

### Step 2: synthesis of 5-(5-amino-4-methylpyridin-2-yl)-7-cyclopropyl-7H-pyrrolo[2,3-d]pyrimidin-4-amine

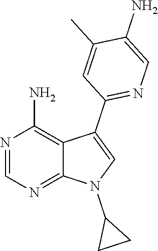 [SMILES: Cc1cc(-c2cn(C3CC3)c3ncnc(N)c23)ncc1N]

The title compound was obtained by following a similar procedure described for Ic-D8, starting from 6-bromo-4-methylpyridin-3-amine (0.142 g, 0.759 mmol) and 7-cyclopropyl-5-(4,4,5,5-tetramethyl-1,3,2-dioxaborolan-2-yl)-7H-pyrrolo[2,3-d]pyrimidin-4-amine (0.251 g, 0.835 mmol), and was obtained as a brown gum (0.05 g, 13% yield). LCMS: 281.0 [M+H].

### Intermediate IC-D14

### 5-(5-aminopyridin-2-yl)-7-cyclopropyl-7H-pyrrolo[2,3-d]pyrimidin-4-amine

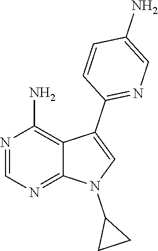 [SMILES: Nc1ccc(-c2cn(C3CC3)c3ncnc(N)c23)nc1]

The title compound was obtained by following a similar procedure described for Ic-D8, starting from 6-bromo-4-methylpyridin-3-amine (0.130 g, 0.751 mmol) and 7-cyclopropyl-5-(4,4,5,5-tetramethyl-1,3,2-dioxaborolan-2-yl)-7H-pyrrolo[2,3-d]pyrimidin-4-amine (step 1 of Intermediate D13, 0.248 g, 0.827 mmol), and was obtained as a brown gum (0.03 g, 4.5% yield) LCMS: 267.0 [M+H].

### Intermediate IC-D15

### 5-(4-amino-3,5-difluorophenyl)-7-cyclopropyl-7H-pyrrolo[2,3-d]pyrimidin-4-amine

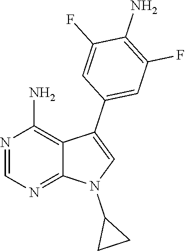 [SMILES: Nc1c(F)cc(-c2cn(C3CC3)c3ncnc(N)c23)cc1F]

The title compound was obtained by following a similar procedure described for Ic-D8, starting from 7-cyclopropyl-5-iodo-7H-pyrrolo[2,3-d]pyrimidin-4-amine (Ic-C1, 0.500 g, 0.751 mmol) and 2,6-difluoro-4-(4,4,5,5-tetramethyl-1,3,2-dioxaborolan-2-yl)aniline (0.425 g, 1.666 mmol), and was obtained as a pale yellow solid (0.10 g, 19% yield). LCMS: 302.1 [M+H].

### Intermediate IC-D16

### 5-(4-amino-2,5-difluorophenyl)-7-cyclopropyl-7H-pyrrolo[2,3-d]pyrimidin-4-amine

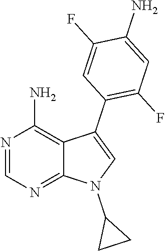 [SMILES: Nc1cc(F)c(-c2cn(C3CC3)c3ncnc(N)c23)cc1F]

The title compound was obtained by following a similar procedure described for Ic-D5, starting from 7-cyclopropyl-5-iodo-7H-pyrrolo[2,3-d]pyrimidin-4-amine (Ic-C1, 0.125 g, 0.417 mmol) and 2,5-difluoro-4-(4,4,5,5-tetramethyl-1,3,2-dioxaborolan-2-yl)aniline (prepared as reported in PCT Pub. No. WO 2017/172093, 0.106 g, 4.17 mmol), and was obtained as a pale yellow solid (0.05 g, 40% yield). LCMS: 302.1 [M+H].

### Intermediate IC-D17

### 5-(4-amino-2,6-difluorophenyl)-7-cyclopropyl-7H-pyrrolo[2,3-d]pyrimidin-4-amine

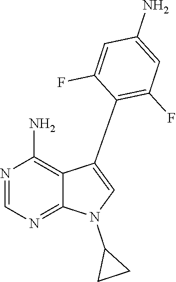 [SMILES: Nc1cc(F)c(-c2cn(C3CC3)c3ncnc(N)c23)c(F)c1]

The title compound was obtained by following a similar procedure described for Ic-D8, starting from 7-cyclopropyl-5-iodo-7H-pyrrolo[2,3-d]pyrimidin-4-amine (Ic-C1, 0.055 g, 0.183 mmol) and 3,5-difluoro-4-(4,4,5,5-tetramethyl-1,3,2-dioxaborolan-2-yl)aniline (prepared as reported in PCT Pub. No. WO 2017/172093, 0.056 g, 0.220 mmol), and was obtained as a pale brown gum (0.046 g) which was used without further purification. LCMS: 301.9 [M+H].

### Intermediate IC-D18

### 1-(4-amino-5-(6-aminopyridin-3-yl)-7H-pyrrolo[2,3-d]pyrimidin-7-yl)-2-methylpropan-2-ol

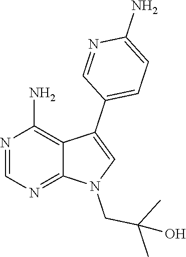 [SMILES: CC(C)(O)Cn1cc(-c2ccc(N)nc2)c2c(N)ncnc21]

The title compound was obtained by following a similar procedure described for Ic-D8, starting from 1-(4-amino-5-iodo-7H-pyrrolo[2,3-<]pyrimidin-7-yl)-2-methylpropan-2-ol (Ic-C3, 0.280 g, 0.733 mmol) and 5-(4,4,5,5-tetramethyl-1,3,2-dioxaborolan-2-yl)pyridin-2-amine (0.161 g, 0.733 mmol), and was obtained as a yellow gum (0.12 g, 50% yield). LCMS: 299.1 [M+H].

### Intermediate IC-D19

### 5-(2-aminopyrimidin-5-yl)-7-cyclopropyl-7H-pyrrolo[2,3-d]pyrimidin-4-amine

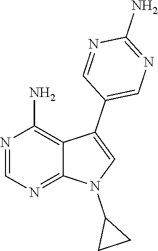 [SMILES: Nc1ncc(-c2cn(C3CC3)c3ncnc(N)c23)cn1]

The title compound was obtained by following a similar procedure described for Ic-D8, starting from 7-cyclopropyl-5-iodo-7H-pyrrolo[2,3-d]pyrimidin-4-amine (Ic-C1, 0.25 g, 0.833 mmol) and 5-(4,4,5,5-tetramethyl-1,3,2-dioxaborolan-2-yl)pyrimidin-2-amine (0.184 g, 0.833 mmol), and was obtained as a colorless gum (0.08 g, 34% yield). LCMS: 268.2 [M+H].

### Intermediate IC-D20

### (2-amino-5-(4-amino-7-cyclopropyl-7H-pyrrolo[2,3-d]pyrimidin-5-yl)phenyl)methanol

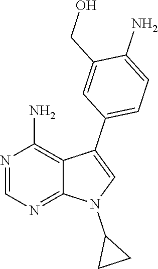 [SMILES: Nc1ccc(-c2cn(C3CC3)c3ncnc(N)c23)cc1CO]

The title compound was obtained by following a similar procedure described for Ic-D8, starting from 7-cyclopropyl-5-iodo-7H-pyrrolo[2,3-d]pyrimidin-4-amine (Ic-C1, 0.200 g, 0.666 mmol) and (2-amino-5-(4,4,5,5-tetramethyl-1,3-dioxolan-2-yl)phenyl)methanol (prepared as reported in PCT Pub. No. WO 2011/130628, 0.184 g, 0.733 mmol), and was obtained as a pale yellow gum (0.025 g, 13% yield). LCMS: 296.0 [M+H].

### Intermediate IC-D21

### 2-amino-5-(4-amino-7-cyclopropyl-7H-pyrrolo[2,3-d]pyrimidin-5-yl)benzonitrile

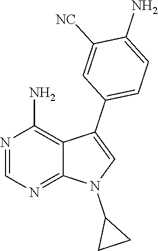 [SMILES: N#Cc1cc(-c2cn(C3CC3)c3ncnc(N)c23)ccc1N]

title compound was obtained by following a similar procedure described for Ic-D8, starting from 7-cyclopropyl-5-iodo-7/N-pyrrolo[2,3-]pyrimidin-4-amine (Ic-C1, 0.250 g, 0.833 mmol) and 2-amino-5-(4,4,5,5-tetramethyl-1,3,2-dioxaborolan-2-yl)benzonitrile (0.203 g, 0.833 mmol), and was obtained as a yellow gum (0.13 g, 34% yield). LCMS: 291.2 [M+H].

### Intermediate IC-D22

### 5-(4-amino-3-fluorophenyl)-7-(2-methoxyethyl)-7H-pyrrolo[2,3-d]pyrimidin-4-amine

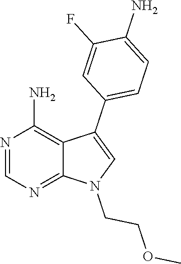 [SMILES: COCCn1cc(-c2ccc(N)c(F)c2)c2c(N)ncnc21]

The title compound was obtained by following a similar procedure described for Ic-D8, starting from 5-iodo-7-(2-methoxyethyl)-7H-pyrrolo[2,3-d]pyrimidin-4-anine (Ic-C8, 0.200 g, 0.629 mmol) and 2-fluoro-4-(4,4,5,5-tetramethyl-1,3,2-dioxaborolan-2-yl)aniline (0.149 g, 0.629 mmol), and was obtained as a pale brown solid (0.12 g, 42% yield). LCMS: 302.2 [M+H].

### Intermediate IC-D23

### 2-(4-amino-5-(4-amine)-3-fluorophenyl)-7H-pyrrolo[2,3-d]pyrimidin-7-yl)ethan-1-ol

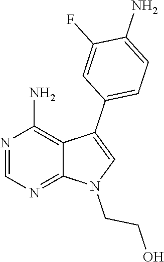 [SMILES: Nc1ccc(-c2cn(CCO)c3ncnc(N)c23)cc1F]

The title compound was obtained by following a similar procedure described for Ic-D8, starting from 2-(4-amino-5-iodo-7H-pyrrolo[2,3-d]pyrimidin-7-yl)ethan-1-ol (Ic-C9, 0.50 g, 1.644 mmol) and 2-fluoro-4-(4,4,5,5-tetramethyl-1,3,2-dioxaborolan-2-yl)aniline (0.39 g, 1.644 mmol), and was obtained as a brown solid (0.3 g, 63% yield). LCMS: 288.1 [M+H].

### Intermediate IC-D24

### 5-(4-amino-3-fluorophenyl)-7-cyclobutyl-7H-pyrrolo[2,3-d]pyrimidin-4-amine

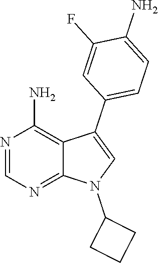 [SMILES: Nc1ccc(-c2cn(C3CCC3)c3ncnc(N)c23)cc1F]

The title compound was obtained by following a similar procedure described for Ic-D8, starting from 7-cyclobutyl-5-iodo-7H-pyrrolo[2,3-<]pyrimidin-4-amine (Ic-C10, 0.410 g, 1.305 mmol) and 2-fluoro-4-(4,4,5,5-tetramethyl-1,3,2-dioxaborolan-2-yl)aniline (0.309 g, 1.305 mmol), and was obtained as a brown solid (0.18 g, 37% yield). ¹H NMR (400 MHz, DMSO-d₆) δ=8 13 (s, 1H), 7.51 (s, 1H), 7.09-7.13 (m, 1H), 6.98-7.01 (m, 1H), 6.84-6.88 (m, 1H), 6.21 (bs, 2H), 5.14-5.26 (m, 3H), 2.67-2.68 (m, 2H), 2.38-2.39 (m, 2H), 1.85-1.86 (m, 2H). LCMS: 298.0 [M+H].

### Intermediate IC-D25

### 5-(4-amino-3-chlorophenyl)-7-cyclopropyl-7H-pyrrolo[2,3-d]pyrimidin-4-amine

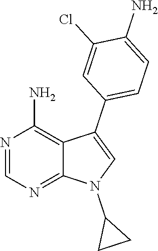 [SMILES: Nc1ccc(-c2cn(C3CC3)c3ncnc(N)c23)cc1Cl]

The title compound was obtained by following a similar procedure described for Ic-D8, starting from 7-cyclopropyl-5-iodo-7H-pyrrolo[2,3-d]pyrimidin-4-amine (Ic-C1, 0.250 g, 0.833 mmol) and 2-chloro-4-(4,4,5,5-tetramethyl-1,3,2-dioxaborolan-2-yl)aniline (0.211 g, 0.833 mmol), and was obtained as a yellow solid (0.03 g, 12% yield). LCMS: 300.1 [M+H].

### Intermediate IC-D26

### 5-(4-amino-3-methoxyphenyl)-7-cyclopropyl-7H-pyrrolo[2,3-d]pyrimidin-4-amine

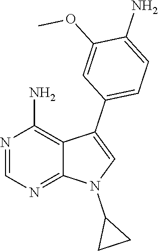 [SMILES: COc1cc(-c2cn(C3CC3)c3ncnc(N)c23)ccc1N]

The title compound was obtained by following a similar procedure described for Ic-D8, starting from 7-cyclopropyl-5-iodo-7H-pyrrolo[2,3-d]pyrimidin-4-amine (Ic-C1, 0.250 g, 0.833 mmol) and 2-methoxy-4-(4,4,5,5-tetramethyl-1,3,2-dioxaborolan-2-yl)aniline (0.208 g, 0.833 mmol), and was obtained as a pale yellow gum (0.04 g, 16% yield). LCMS: 296.1 [M+H].

### Intermediate IC-D27

### 5-(4-amino-3-fluorophenyl)-7-(1-methylpyrrolidin-3-yl)-7H-pyrrolo[2,3-d]pyrimidin-4-amine

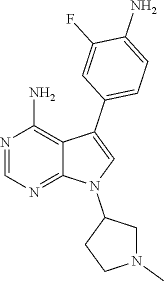 [SMILES: CN1CCC(n2cc(-c3ccc(N)c(F)c3)c3c(N)ncnc32)C1]

The title compound was obtained by following a similar procedure described for Ic-D8, starting from 5-iodo-7-(1-methylpyrrolidin-3-yl)-7H-pyrrolo[2,3-d]pyrimidin-4-amine (Ic-C11, 0.155 g, 0.452 mmol) and 2-fluoro-4-(4,4,5,5-tetramethyl-1,3,2-dioxaborolan-2-yl)aniline (0.107 g, 0.452 mmol), and was obtained as a brown solid (0.084 g, 27% yield). LCMS: 327.2 [M+H].

### Intermediate IC-E1 through IC-E25

### General Procedure for the Synthesis of Carbamate Intermediates Ic-E:

Pyridine (1.2 eq) and phenyl chloroformate (1.5 eq) were added to a solution of amine (1.0 eq) in THF (10 vol) at 0° C. The reaction mixture was allowed to warm to 25° C. and was stirred for 12 h. Following completion of the reaction (as indicated by TLC), the mixture was diluted with EtOAc (10 mL) and washed with brine (5 mL). The organic layer was dried over Na₂SO₄, filtered, and concentrated under reduced pressure to yield crude material which was purified by flash chromatography (silica gel 230-400 mesh, eluting with 10 to 20% EtOAc in petroleum), giving the desired carbamate.

The following carbamates were prepared using the above general procedure:

| Intermediate | Carbamate structure (product) | Amine structure (starting material) | LCMS |
|---|---|---|---|
| Ic-E1 | 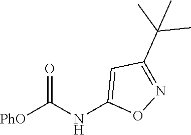 [SMILES: CC(C)(C)c1cc(NC(=O)Oc2ccccc2)on1] | 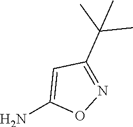 | 261.0 [M + H] |
| Ic-E2 | 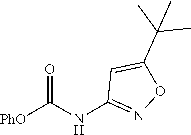 [SMILES: CC(C)(C)c1cc(NC(=O)Oc2ccccc2)no1] | 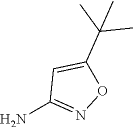 | 261.0 [M + H] |
| Ic-E3 | 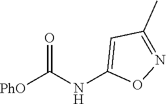 [SMILES: Cc1cc(NC(=O)Oc2ccccc2)on1] | 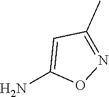 | 219.1 [M + H] |
| Ic-E4 | 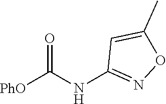 [SMILES: Cc1cc(NC(=O)Oc2ccccc2)no1] | 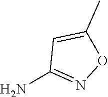 | 219.1 [M + H] |
| Ic-E5 | 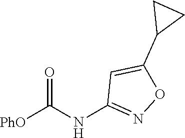 [SMILES: O=C(Nc1cc(C2CC2)on1)Oc1ccccc1] | 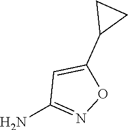 | 245.0 [M + H] |
| Ic-E6 | 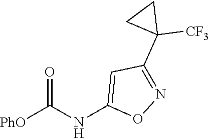 [SMILES: O=C(Nc1cc(C2(C(F)(F)F)CC2)no1)Oc1ccccc1] | 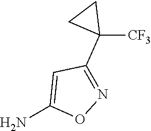 [SMILES: Nc1cc(C2(C(F)(F)F)CC2)no1] | 312.9 [M + H] |
| Ic-E7 | 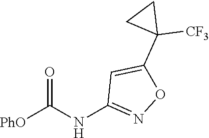 [SMILES: O=C(Nc1cc(C2(C(F)(F)F)CC2)on1)Oc1ccccc1] | 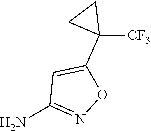 [SMILES: Nc1cc(C2(C(F)(F)F)CC2)on1] | 312.9 [M + H] |
| Ic-E8 | 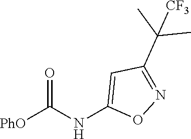 [SMILES: CC(C)(c1cc(NC(=O)Oc2ccccc2)on1)C(F)(F)F] | 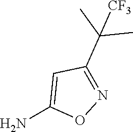 | 315.1 [M + H] |
| Ic-E9 | 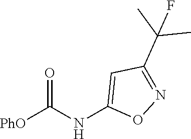 [SMILES: CC(C)(F)c1cc(NC(=O)Oc2ccccc2)on1] | 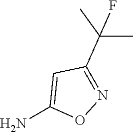 | 265.1 [M + H] |
| Ic-E10 | 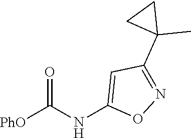 [SMILES: CC1(c2cc(NC(=O)Oc3ccccc3)on2)CC1] | 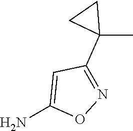 | 259.1 [M + H] |
| Ic-E11 | 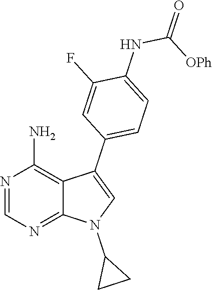 [SMILES: Nc1ncnc2c1c(-c1ccc(NC(=O)Oc3ccccc3)c(F)c1)cn2C1CC1] | 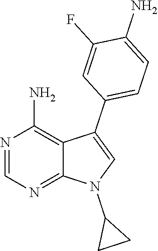 [SMILES: Nc1ccc(-c2cn(C3CC3)c3ncnc(N)c23)cc1F] | 404.0 [M + H] |
| Ic-E12 | 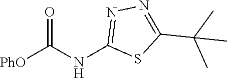 [SMILES: CC(C)(C)c1nnc(NC(=O)Oc2ccccc2)s1] | 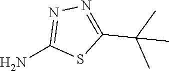 | 278.0 [M + H] |
| Ic-E13 | 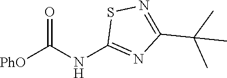 [SMILES: CC(C)(C)c1nsc(NC(=O)Oc2ccccc2)n1] | 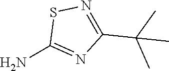 | 277.1 [M] |
| Ic-E14 | 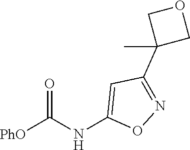 [SMILES: CC1(c2cc(NC(=O)Oc3ccccc3)on2)COC1] | 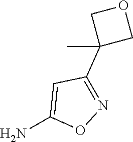 | 273.0 [M − H] |
| Ic-E15 | 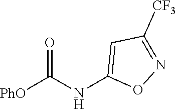 [SMILES: O=C(Nc1cc(C(F)(F)F)no1)Oc1ccccc1] | 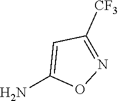 | 270.9 [M − H] |
| Ic-E16 | 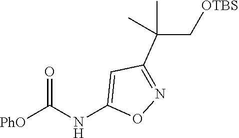 [SMILES: CC(C)(CO[Si](C)(C)C(C)(C)C)c1cc(NC(=O)Oc2ccccc2)on1] | 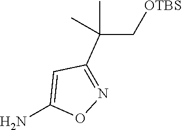 | 391.2 [M + H] |
| Ic-E17 | 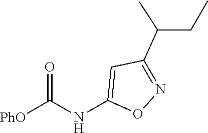 [SMILES: CCC(C)c1cc(NC(=O)Oc2ccccc2)on1] | 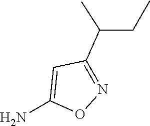 | 261.0 [M + H] |
| Ic-E18 | 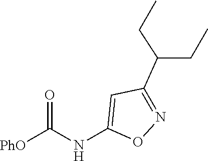 [SMILES: CCC(CC)c1cc(NC(=O)Oc2ccccc2)on1] | 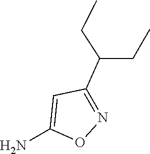 | 273.0 [M − H] |
| Ic-E19 | 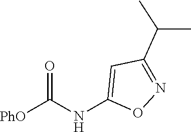 [SMILES: CC(C)c1cc(NC(=O)Oc2ccccc2)on1] | 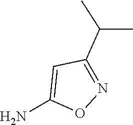 | 247.1 [M + H] |
| Ic-E20 | 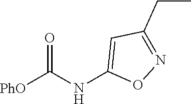 [SMILES: CCc1cc(NC(=O)Oc2ccccc2)on1] | 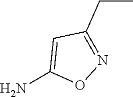 | 233.0 [M + H] |
| Ic-E21 | 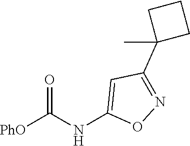 [SMILES: CC1(c2cc(NC(=O)Oc3ccccc3)on2)CCC1] | 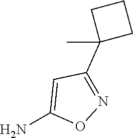 | 272.5 [M + H] |
| Ic-E22 | 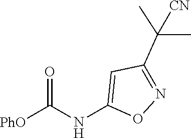 [SMILES: CC(C)(C#N)c1cc(NC(=O)Oc2ccccc2)on1] | 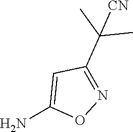 | 272.5 [M + H] |
| Ic-E23 | 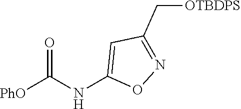 | 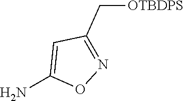 | 349.6 [M + H] |
| Ic-E24 | 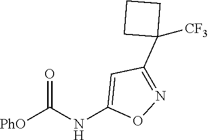 [SMILES: O=C(Nc1cc(C2(C(F)(F)F)CCC2)no1)Oc1ccccc1] | 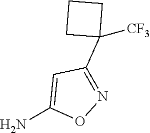 [SMILES: Nc1cc(C2(C(F)(F)F)CCC2)no1] | 327.1 [M + H] |
| Ic-E25 | 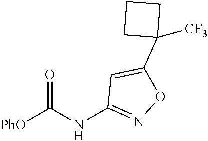 [SMILES: O=C(Nc1cc(C2(C(F)(F)F)CCC2)on1)Oc1ccccc1] | 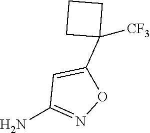 [SMILES: Nc1cc(C2(C(F)(F)F)CCC2)on1] | 327.0 [M + H] |

All amines used for the synthesis of carbamate Intermediates Ic-E are commercially available except for the following:

3-(1-(Trifluoromethyl)cyclopropyl)isoxazol-5-anine (precursor to Ic-E6) and 5-(1-(trifluoromethyl)cyclopropyl)isoxazol-3-amine (precursor to Ic-E7) were synthesized as reported in Synthesis 2013, 45, 171-173.

3-(1,1,1-Trifluoro-2-methylpropan-2-yl)isoxazol-5-amine (precursor to Ic-E8) and 3-(2-fluoropropan-2-yl)isoxazol-5-amine (precursor to Ic-E9) were synthesized from methyl 3,3,3-trifluoro-2,2-dimethylpropanote and methyl 2-fluoro-2-methylpropionate, respectively, followed by the procedure reported in Synthesis 2013, 45, 171-173.

3-(1-((Tert-butyldimethylsilyl)oxy)-2-methylpropan-2-yl)isoxazol-5-anine (precursor to Ic-E16) was synthesized as reported in PCT publication No. WO 2010/036630.

2-(5-Aminoisoxazol-3-yl)-2-methylpropanenitrile (precursor to Ic-E22) and 3-(1-(Trifluoromethyl)cyclobutyl)isoxazol-5-amine (precursor to Ic-E24) were synthesized as reported in J. Med. Chem. 2012, 55(3), 1082-1105.

3-(((Tert-butyldiphenylsilyl)oxy)methyl)isoxazol-5-amine (precursor to Ic-E23) was synthesized as reported in PCT publication No WO 2013/104561.

5-(1-(Trifluoromethyl)cyclobutyl)isoxazol-3-amine (precursor to Ic-E25) was synthesized as reported in PCT publication No. WO 2011/022473.

Synthesis of 3-(3-methyloxetan-3-yl)isoxazol-5-amine (precursor to Ic-E14).

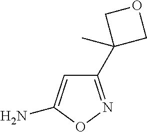

NH₂OH·H₂SO₄ (0.520 g, 3.16 mmol) was added to a solution of 3-(3-methyloxetan-3-yl)-3-oxopropanenitrile (prepared as reported in PCT Pub No. WO 2019/192962, 0.400 g, 2.87 mmol) and sodium hydroxide (0.126 g, 3.16 mmol) in EtOH (10 mL) and water (10 mL). The pH of the resulting mixture was adjusted to 7.5 using aqueous NaOH (1 M) and the reaction mixture was stirred at 80° C. for 15 h. Following completion of the reaction (as indicated by TLC), the reaction mixture was concentrated under reduced pressure, giving a residue which was taken in EtOAc (25 mL), washed with water (10 mL), dried over Na₂SO₄, filtered, and concentrated under reduced pressure. The resulting crude material was purified by flash chromatography (silica gel 230-400 mesh, eluting with 30% EtOAC in petroleum ether to afford the title product as a pale brown solid (0.09 g, 20% yield). ¹H NMR (400 MHz, CDCl₃) δ=5.21 (s, 1H), 4.90-4.93 (m, 2H), 4.56-4.59 (m, 2H), 1.70 (s, 3H). LCMS: 155.1 [M+H].

### Synthesis of 3-(1-methylcyclobutyl)isoxazol-5-amine (precursor to Ic-E2H)

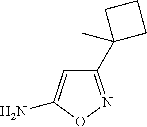

NH₂OH·H₂SO₄ (0.699 g, 4.25 mmol) was added to a solution of 3-(1-methylcyclobutyl)-3-oxopropanenitrile (prepared as reported in PCT Pub No. WO 2017/060874, 0.500 g, 3.86 mmol) and sodium hydroxide (0.170 g, 4.25 mmol) in EtOH (10 mL) and water (10 mL). The pH of the resulting mixture was adjusted to 7.5 using aqueous NaOH (1M) and the reaction mixture was stirred at 80° C. for 15 h. Following completion of the reaction (as indicated by TLC), the reaction mixture was concentrated under reduced pressure, giving a residue which was taken in DCM (25 mL), washed with water (10 mL), dried over Na₂SO₄, filtered, and concentrated under reduced pressure. The resulting crude material was purified by flash chromatography (silica gel 230-400 mesh, eluting with 40% EtOAc in petroleum ether to afford the title product as an off-white solid (0.110 g, 19% yield). ¹H NMR (400 MHz, CDCl₃) δ=5.04 (s, 1H), 2.43-2.49 (m, 2H), 1.96-2.02 (m, 4H), 1.50 (s, 3H). LCMS: 153.2 [M+H].

### Preparation of Examples

General Urea Formation Procedure for the Synthesis of Examples Ic-1 Through Ic-62

Method Ic-A—Triethylamine (2.0 eq.) was added to a mixture of amine Intermediate Ic-D (1.0 eq.) and carbamate intermediate Ic-E (1.0 eq) in THF (10 Vol) and the resulting mixture was stirred at 60° C. for 12 h in a sealed tube. Following completion of the reaction (as indicated by LCMS), the reaction mixture was concentrated under reduced pressure to give crude material which was purified by reverse phase preparative HPLC to afford the desired product.

Method Ic-B— DMAP (0.05 eq.) and DIPEA (1.5 eq.) were added to a solution of amine Intermediate Ic-D (1.0 eq.) and carbamate Intermediate Ic-E (1.0 eq.) in THF (10 Vol) and the resulting mixture was stirred at 60° C. for 12 h in a sealed tube. Following completion of the reaction (as indicated by LCMS), the reaction mixture was concentrated under reduced pressure to yield the crude material which was purified by reverse phase preparative HPLC to afford the desired product.

The following compounds were prepared using the above general procedures.

### Example IC-1

### 1-(4-(4-amino-7-(oxetan-3-yl)-7H-pyrrolo[2,3-d]pyrimidin-5-yl)phenyl)-3-(3-(tert-butyl)isoxazol-5-yl)urea

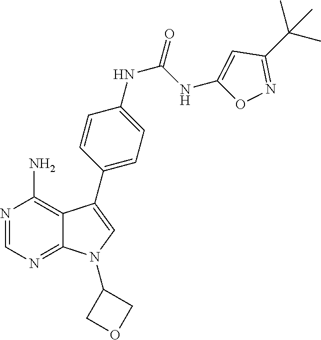 [SMILES: CC(C)(C)c1cc(NC(=O)Nc2ccc(-c3cn(C4COC4)c4ncnc(N)c34)cc2)on1]

The title compound was prepared following the general procedure for urea formation (Method Ic-A), starting from 5-(4-aminophenyl)-7-(oxetan-3-yl)-7H-pyrrolo[2,3-d]pyrimidin-4-amine (Ic-D2, 0.178 g, 0.63 mmol) and phenyl (3-tert-butyl)isoxazol-5-yl)carbamate (Ic-E1, 0.16 g, 0.30 mmol), and was obtained as an off-white solid (0.026 g, 9% yield). ¹H NMR (400 MHz, DMSO-d₄) δ=10.48 (bs, H), 9.34 (bs, 1H), 8.15 (s, 1H), 7.70 (s, 1H), 7.61 (d, J=8.8 Hz, 2H), 7.45 (d, J=8.8 Hz, 2H), 6.08 (bs, 2H), 6.07 (s, 1H), 5.86-5.90 (m, 1H), 4.97-5.05 (m, 4H), 1.27 (s, 9H). LCMS: 448.2 [M+H].

### Example IC-2

### 1-(4-(4-amino-7-cyclopropyl-7H-pyrrolo[2,3-d]pyrimidin-5-yl)-2-fluorophenyl)-3-(3-(tert-butyl)isoxazol-5-yl)urea

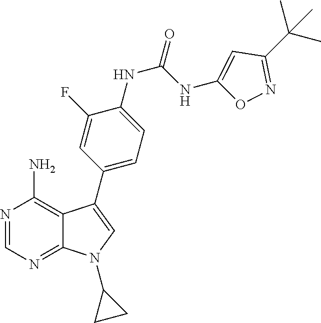 [SMILES: CC(C)(C)c1cc(NC(=O)Nc2ccc(-c3cn(C4CC4)c4ncnc(N)c34)cc2F)on1]

The title compound was prepared following the general procedure for urea formation (Method Ic-A), starting from 5-(4-aminophenyl)-7-(oxetan-3-yl)-7H-pyrrolo[2,3-d]pyrimidin-4-amine (Ic-D2, 0.178 g, 0.63 mmol) and phenyl (3-(tert-butyl)isoxazol-5-yl)carbamate (Ic-E1, 0.16 g, 0.30 mmol), and was obtained as an off-white solid (0.026 g, 9% yield). ¹H NMR (400 MHz, DMSO-d₆) δ=10.48 (bs, 1H), 9.34 (bs, 1H), 8.15 (s, 1H), 7.70 (s, 1H), 7.61 (d, J=8.8 Hz, 2H), 7.45 (d, J=8.8 Hz, 2H), 6.08 (bs, 2H), 6.07 (s, 1H), 5.86-5.90 (m, 1H), 4.97-5.05 (m, 4H), 1.27 (s, 9H). LCMS: 448.2 [M+H].

### Example IC-3

### 1-(5-(4-amino-7-cyclopropyl-7H-pyrrolo[2,3-d]pyrimidin-5-yl)pyridin-2-yl)-3-(3-(tert-butyl)isoxazol-5-yl)urea

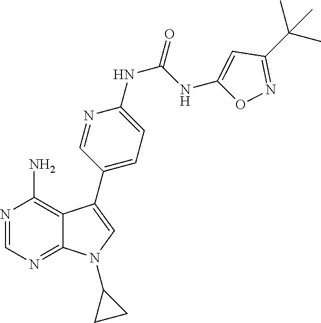 [SMILES: CC(C)(C)c1cc(NC(=O)Nc2ccc(-c3cn(C4CC4)c4ncnc(N)c34)cn2)on1]

The title compound was prepared following the general procedure for urea formation (Method Ic-A), starting from 5-(6-aminopyridin-3-yl)-7-cyclopropyl-7H-pyrrolo[2,3-d]pyrimidin-4-amine (Ic-D6, 0.100 g, 0.376 mmol) and phenyl (3-(tert-butyl)isoxazol-5-yl)carbamate (Ic-E1, 0.098 g, 0.376 mmol), and was obtained as an off-white solid (9.6 mg, 6% yield). ¹H NMR (400 MHz, CD₃OD) δ=8.46-8.47 (m, 1H), 8.22 (s, 1H), 7.89-7.92 (m, 2H), 7.35-7.38 (m, 1H), 7.28 (s, 1H), 6.23 (s, 1H), 3.50-3.57 (m, 1H), 1.27 (s, 9H), 1.07-1.16 (m, 4H); LCMS: 433.2 [M+H].

### Example IC-4

### 1-(4-(4-amino-7-cyclopropyl-7H-pyrrolo[2,3-d]pyrimidin-5-yl)-2-fluorophenyl)-3-(5-(tert-butyl)isoxazol-3-yl)urea

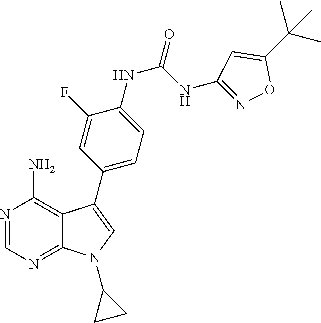 [SMILES: CC(C)(C)c1cc(NC(=O)Nc2ccc(-c3cn(C4CC4)c4ncnc(N)c34)cc2F)no1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.100 g, 0.35 mmol)) and phenyl (5-(tert-butyl)isoxazol-3-yl)carbamate (Ic-E2, 0.091 g, 0.35 mmol), and was obtained as an off-white solid (0.021 g, 13% yield). ¹H NMR (400 MHz, DMSO-d₆) δ=9.86 (bs, 1H), 8.87 (bs, 1H), 8.17-8.21 (m, 2H), 7.24-7.35 (m, 3H), 6.51 (s, 1H), 6.17 (bs, 2H), 3.56-3.60 (m, 1H), 1.31 (s, 9H), 1.02-1.07 (m, 4H). LCMS: 450.2 [M+H].

### Example IC-5

### 1-(4-(4-amino-7-Cyclopropyl-7H-pyrrolo[2,3-d]pyrimidin-5-Yl)-2-fluorophenyl)-3-(3-(1-(trifluoromethyl)cyclopropyl)isoxazol-5-yl)urea

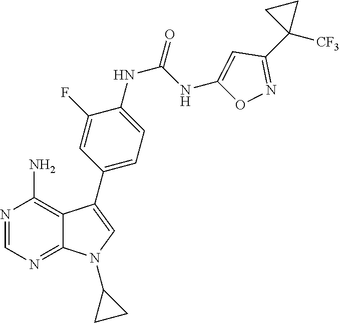 [SMILES: Nc1ncnc2c1c(-c1ccc(NC(=O)Nc3cc(C4(C(F)(F)F)CC4)no3)c(F)c1)cn2C1CC1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.080 g, 0.282 mmol) and phenyl (3-(1-(trifluoromethyl)cyclopropyl)isoxazol-5-yl)carbamate (Ic-E6, 0.088 g, 0.282 mmol), and was obtained as a white solid (0.031 g, 22% yield). ¹H NMR (400 MHz, DMSO-d₆) δ=10.59 (bs, 1H), 8.84 (bs, 1H), 8.11-8.17 (m, 2H), 7.26-7.37 (m, 3H), 6.20 (s, 1H), 6.16 (bs, 2H), 3.55-3.61 (m, 1H), 1.45-1.49 (m, 2H), 1.38-1.43 (m, 2H), 1.03-1.08 (m, 4H). LCMS: 502.1 [M+H].

### Example IC-6

### 1-(4-(4-amino-7-cyclopropyl-7H-pyrrolo[2,3-d]pyrimidin-5-yl)-2-fluorophenyl)-3-(3-(1-(trifluoromethyl)cyclopropyl)isoxazol-5-yl)urea

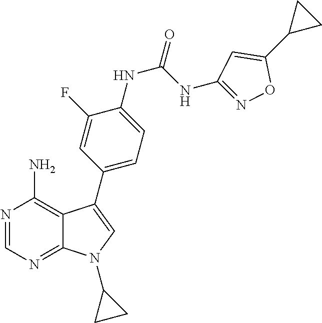 [SMILES: Nc1ncnc2c1c(-c1ccc(NC(=O)Nc3cc(C4CC4)on3)c(F)c1)cn2C1CC1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.060 g, 0.180 mmol) and phenyl (5-cyclopropylisoxazol-3-yl)carbamate (Ic-E5, 0.044 g, 0.282 mmol), and was obtained as an off-white solid (8.4 mg, 9% yield). ¹H NMR (400 MHz, CD₃OD) δ=8.20-8.22 (m, 2H), 7.28-7.33 (m, 2H), 7.22 (s, 1H), 6.35 (s, 1H), 3.49-3.55 (m, 1H), 2.08-2.12 (m, 1H), 1.08-1.16 (m, 6H), 0.97-0.99 (m, 2H). LCMS: 434.2 [M+H].

### Example IC-7

### 1-(4-(4-amino-7-cyclopropyl-7H-pyrrolo[2,3-d]pyrimidin-5-yl)-2-fluorophenyl)-3-(3-methylisoxazol-5-yl)urea

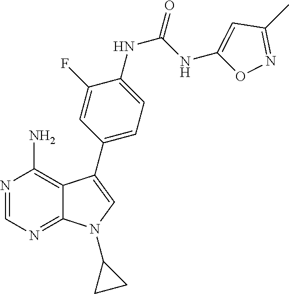 [SMILES: Cc1cc(NC(=O)Nc2ccc(-c3cn(C4CC4)c4ncnc(N)c34)cc2F)on1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.100 g, 0.35 mmol)) and phenyl (3-methylisoxazol-5-yl)carbamate (Ic-E3, 0.077 g, 0.35 mmol), and was obtained as an off-white solid (0.024 g, 17% yield). ¹H NMR (400 MHz, DMSO-d₆) δ=10.35 (bs, 1H), 8.84 (bs, 1H), 8.10-8.18 (m, 2H), 7.25-7.36 (m, 3H), 6.13 (bs, 2H), 5.99 (s, 1H), 3.55-3.61 (m, 1H), 2.18 (s, 3H), 1.00-1.08 (m, 4H). LCMS: 408.1 [M+H].

### Example IC-8

### 1-(4-(4-amino-7-cyclopropyl-7H-pyrrolo[2,3-d]pyrimidin-5-yl)-2-fluorophenyl)-3-(5-methylisoxazol-3-yl)urea

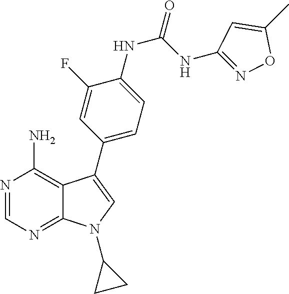 [SMILES: Cc1cc(NC(=O)Nc2ccc(-c3cn(C4CC4)c4ncnc(N)c34)cc2F)no1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.127 g, 0.44 mmol) and phenyl (5-methylisoxazol-3-yl)carbamate (Ic-E4, 0.097 g, 0.44 mmol), and was obtained as a white solid (0.033 g, 18% yield). ¹H NMR (400 MHz, DMSO-d₆) δ=9.88 (bs, 1H), 8.96 (bs, 1H), 8.15-8.19 (m, 2H), 7.24-7.35 (m, 3H), 6.54 (s, 1H), 6.15 (bs, 2H), 3.55-3.61 (m, 1H), 2.38 (s, 3H), 1.00-1.07 (m, 4H). LCMS: 408.2 [M+H].

### Example IC-9

### 1-(4-(4-amino-7-cyclopropyl-7H-pyrrolo[2,3-d]pyrimidin-5-yl)-2-fluorophenyl)-3-(3-(2-fluoropropan-2-yl)isoxazol-5-yl)urea

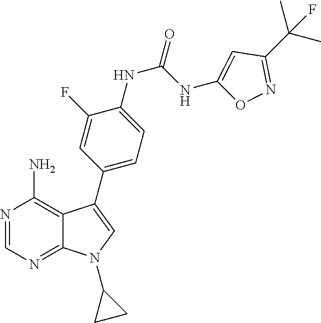 [SMILES: CC(C)(F)c1cc(NC(=O)Nc2ccc(-c3cn(C4CC4)c4ncnc(N)c34)cc2F)on1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.076 g, 0.26 mmol) and phenyl (3-(2-fluoropropan-2-yl)isoxazol-5-yl)carbamate (Ic-E9, 0.070 g, 0.26 mmol), and was obtained as a white solid (0.018 g, 14% yield). ¹H NMR (400 MHz, DMSO-d₆) δ=8.99 (bs, 1H), 8.17 (s, 1H), 8.09-8.14 (m, 1H), 7.25-7.36 (m, 3H), 6.18 (s, 1H), 6.09 (bs, 2H), 3.57-3.61 (m, 1H), 1.71 (s, 3H), 1.66 (s, 3H), 1.02-1.05 (m, 4H). LCMS: 454.2 [M+H].

### Example IC-10

### 1-(4-(4-amino-7-cyclopropyl-7H-pyrrolo[2,3-d]pyrimidin-5-yl)-2-fluorophenyl)-3-(5-(3-(1-(trifluoromethyl)cyclopropyl)isoxazol-3-yl)urea

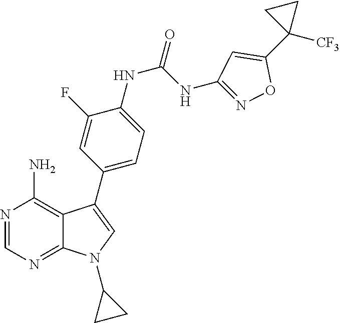 [SMILES: Nc1ncnc2c1c(-c1ccc(NC(=O)Nc3cc(C4(C(F)(F)F)CC4)on3)c(F)c1)cn2C1CC1]

The title compound was prepared following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.10 g, 0.35 mmol) and phenyl (5-(1-(trifluoromethyl)cyclopropyl)isoxazol-3-yl)carbamate (Ic-E7, 0.11 g, 0.35 mmol), and was obtained as an off-white solid (0.010 g, 6% yield). ¹H NMR (400 MHz, DMSO-d₆) δ=9.99 (bs, 1H), 8.86 (bs, 1H), 8.14-8.18 (m, 2H), 7.24-7.36 (m, 3H), 6.90 (s, 1H), 6.15 (bs, 2H), 3.56-3.61 (m, 1H), 1.48-1.57 (m, 4H), 1.02-1.07 (m, 4H). LCMS: 502.2 [M+H].

### Example IC-11

### 1-(4-(4-amino-7-cyclopropyl-7H-pyrrolo[2,3-d]pyrimidin-5-yl)-3-fluorophenyl)-3-(3-(1-(trifluoromethyl)cyclopropyl)isoxazol-5-yl)urea

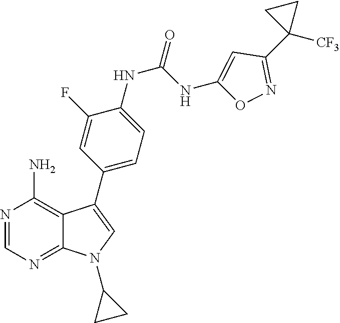 [SMILES: Nc1ncnc2c1c(-c1ccc(NC(=O)Nc3cc(C4(C(F)(F)F)CC4)no3)c(F)c1)cn2C1CC1]

The title compound was prepared following the general procedure for urea formation (Method Ic-A), starting from 5-(4-amino-2-fluorophenyl)-7-cyclopropyl-7H-pyrrolo[2,3-d]pyrimidin-4-amine (Ic-D5, 0.070 g, 0.24 mmol) and phenyl (3-(1-(trifluoromethyl)cyclopropyl)isoxazol-5-yl)carbamate (Ic-E6, 0.082 g, 0.24 mmol), and was obtained as an off-white solid (0.011 g, 9% yield). ¹H NMR (400 MHz, DMSO-d₆) δ=10.56 (bs, 1H), 9.29 (bs, 1H), 8.16 (s, 1H), 7.58-7.62 (m, 1H), 7.27-7.36 (m, 2H), 7.22 (s, 1H), 6.20 (s, 1H), 6.00 (bs, 2H), 3.55-3.60 (m, 1H), 1.38-1.48 (m, 4H), 1.02-1.06 (m, 4H) LCMS: 502.2 [M+H].

### Example IC-12

### 1-(4-(4-amino-7-cyclopropyl-7H-pyrrolo[2,3-d]pyrimidin-5-yl)-2-fluorophenyl)-3-(3-(1,1,1-Trifluoro-2-methylpropan-2-yl)isoxazol-5-yl)urea

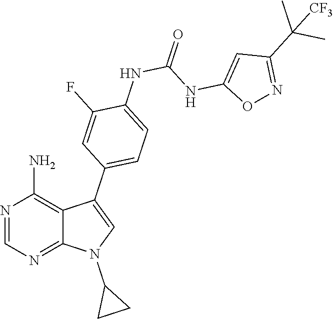 [SMILES: CC(C)(c1cc(NC(=O)Nc2ccc(-c3cn(C4CC4)c4ncnc(N)c34)cc2F)on1)C(F)(F)F]

The title compound was prepared following the general procedure for urea formation (Method Ic-A), starting from 5-(4-amino-2-fluorophenyl)-7-cyclopropyl-7H-pyrrolo[2,3-d]pyrimidin-4-amine (Ic-D1, 0.050 g, 0.176 mmol) and phenyl (3-(1,1,1-trifluoro-2-methylpropan-2-yl)isoxazol-5-yl)carbamate (Ic-E8, 0.055 g, 0.176 mmol), and was obtained as an off-white solid (9.9 mg, 11% yield). ¹H NMR (400 MHz, DMSO-d₆) δ=10.59 (bs, 1H), 8.83 (bs, 1H), 8.18 (s, 1H), 8.12-8.17 (m, 1H), 7.25-7.36 (m, 3H), 6.22 (s, 1H), 6.17 (bs, 2H), 3.55-3.61 (m, 1H), 1.52 (s, 6H), 1.02-1.07 (m, 4H). LCMS: 504.2 [M+H].

### Example IC-13

### 1-(4-(4-amino-7-cyclopropyl-7H-pyrrolo[2,3-d]pyrimidin-5-yl)phenyl)-3-(3-(tert-butyl)isoxazol-5-yl)urea

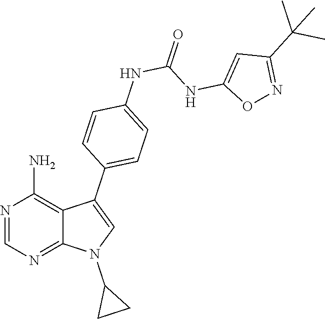 [SMILES: CC(C)(C)c1cc(NC(=O)Nc2ccc(-c3cn(C4CC4)c4ncnc(N)c34)cc2)on1]

The title compound was prepared following the general procedure for urea formation (Method Ic-A), starting from 5-(4-aminophenyl)-7-cyclopropyl-7H-pyrrolo[2,3-<]pyrimidin-4-amine (Ic-D4, 0.080 g, 0.30 mmol) and phenyl (3-(tert-butyl)isoxazol-5-yl)carbamate (Ic-E1, 0.078 g, 0.30 mmol), and was obtained as an off-white solid (0.036 g, 28% yield). ¹H NMR (400 MHz, DMSO-d₆) δ=10.13 (bs, 1H), 8.93 (bs, 1H), 8.16 (s, 1H), 7.56 (d, J=8.4 Hz, 2H), 7.40 (d, J=8.8 Hz, 2H), 7.22 (s, 1H), 6.08 (s, 1H), 6.00 (bs, 2H), 3.56-3.59 (m, 1H), 1.27 (s, 9H), 1.05-1.07 (m, 4H). LCMS: 430.2 [M−H].

### Example IC-14

### 1-(4-(4-amino-7-(2-hydroxy-2-methyl propyl)-7H-pyrrolo[2,3-d]pyrimidin-5-yl)-2-fluorophenyl)-3-(3-(1-(trifluoromethyl)cyclopropyl)isoxazol-5-yl)urea

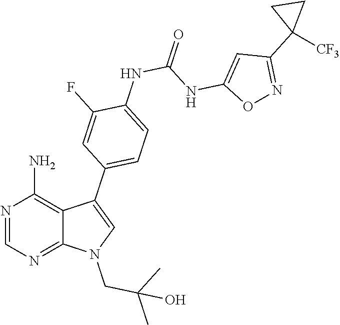 [SMILES: CC(C)(O)Cn1cc(-c2ccc(NC(=O)Nc3cc(C4(C(F)(F)F)CC4)no3)c(F)c2)c2c(N)ncnc21]

The title compound was prepared following the general procedure for urea formation (Method Ic-A), starting from 1-(4-amino-5-(4-amino-3-fluorophenyl)-7H-pyrrolo[2,3-d]pyrimidin-7-yl)-2-methylpropan-2-ol (Ic-D3, 0.086 g, 0.273 mmol) and phenyl (3-(1-(trifluoromethyl)cyclopropyl)isoxazol-5-yl)carbamate (Ic-E6, 0.085 g, 0.273 mmol), and was obtained as pale brown solid (0.011 g, 8% yield). ¹H NMR (400 MHz, DMSO-d₆) δ=10.92 (bs, 1H), 9.06 (bs, 1H), 8.11-8.15 (m, 2H), 7.26-7.35 (m, 3H), 6.18 (s, 1H), 6.14 (bs, 2H), 4.86 (bs, 1H), 4.11 (bs, 2H), 1.37-1.46 (m, 4H), 1.06-1.08 (m, 6H). LCMS: 534.1 [M+H].

### Example IC-15

### 1-(4-(4-amino-7-cyclopropyl-7H-pyrrolo[2,3-d]pyrimidin-5-yl)cyclohex-3-EN-1-yl)-3-(3-(tert-butyl)isoxazol-5-yl)urea

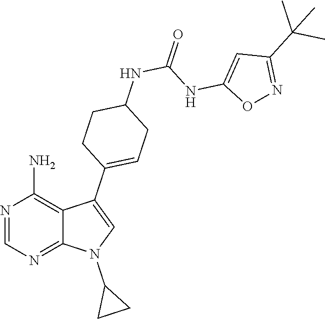 [SMILES: CC(C)(C)c1cc(NC(=O)NC2CC=C(c3cn(C4CC4)c4ncnc(N)c34)CC2)on1]

The title compound was prepared following the general procedure for urea formation (Method Ic-A), starting from 5-(4-aminocyclohex-1-en-1-yl)-7-cyclopropyl-7H-pyrrolo[2,3-d]pyrimidin-4-amine (Ic-D7, 0.120 g, 0.446 mmol) and phenyl (3-tert-butyl)isoxazol-5-yl)carbamate (Ic-E1, 0.116 g, 0.446 mmol), and was obtained as an off-white solid (0.013 g, 7% yield). ¹H NMR (400 MHz, DMSO-d₆) δ=9.83 (bs, 1H), 8.39 (bs, 1H), 7.47 (s, 1H), 6.54 (d, J=7.6 Hz, 1H), 5.93 (s, 1H), 5.67 (bs, 1H), 3.88-3.90 (m, 2H), 3.61-3.67 (m, 2H), 2.08-2.14 (m, 1H), 1.92-1.95 (m, 1H), 1.69-1.73 (m, 1H), 1.24 (s, 9H), 1.05-1.08 (m, 4H). LCMS: 436.2 [M+H].

### Example IC-16

### 1-(4-(4-amino-7-cyclopropyl-7H-pyrrolo[2,3-d]pyrimidin-5-yl)cyclohexyl)-3-(3-(tert-butyl)isoxazol-5-yl)urea

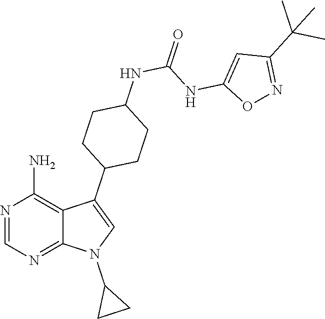 [SMILES: CC(C)(C)c1cc(NC(=O)NC2CCC(c3cn(C4CC4)c4ncnc(N)c34)CC2)on1]

Platinum oxide (0.016 g, 0.069 mmol) was added to a solution of 1-(4-(4-amino-7-cyclopropyl-7H-pyrrolo[2,3-d]pyrimidin-5-yl)cyclohex-3-en-1-yl)-3-(3-(tert-butyl)isoxazol-5-yl)urea (Example Ic-15, 0.100 g, 0.230 mmol) in EtOAc (5 mL) and the resulting suspension was stirred at room temperature under H₂ atmosphere for 12 h. Following completion of the reaction (as indicated by LCMS), the reaction mixture was filtered through a pad of diatomaceous earth which was then rinsed with EtOAc (2-5 mL). The combined filtrates were concentrated under reduced pressure to yield crude material which was purified by preparative HPLC (mass-based, eluting with a gradient of ammonium acetate in water and ACN), giving the title product as an off white solid (2.0 mg, 2% yield). ¹H NMR (400 MHz, CD₃OD) δ=8.12 (s, 1H), 6.92 (s, 1H), 6.02 (s, 1H), 3.50-3.51 (m, 1H), 2.87-2.90 (m, 1H), 2.13-2.18 (m, 6H), 1.51-1.57 (m, 3H), 1.33 (s, 9H), 0.90-1.20 (m, 4H). LCMS: 436.2 [M−H].

### Example IC-17

### 1-(4-(4-amino-7-cyclopropyl-7H-pyrrolo[2,3-d]pyrimidin-5-yl)-2-fluorophenyl)-3-(3-(1-methylcyclopropyl)isoxazol-5-yl)urea

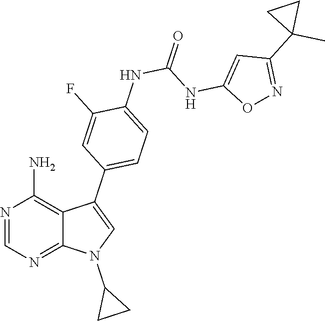 [SMILES: CC1(c2cc(NC(=O)Nc3ccc(-c4cn(C5CC5)c5ncnc(N)c45)cc3F)on2)CC1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.050 g, 0.176 mmol) and phenyl (3-(1-methylcyclopropyl)isoxazol-5-yl)carbamate (Ic-E10, 0.046 g, 0.176 mmol), and was obtained as an off-white solid (0.013 g, 16% yield). ¹H NMR (400 MHz, DMSO-d₆) δ=40 (bs, 1H), 8.84 (bs, 1H), 8.11-8.17 (m, 2H), 7.24-7.36 (m, 3H), 6.16 (bs, 2H), 5.84 (s, 1H), 3.55-3.61 (m, 1H), 1.38 (s, 3H), 1.02-1.07 (m, 4H), 0.94-0.96 (m, 2H), 0.83-0.84 (m, 2H). LCMS: 448.2 [M+H].

### Example IC-18

### 1-(4(4-amino-7-cyclopropyl-7H-pyrrolo[2,3-d]pyrimidin-5-yl)-2-fluorophenyl)-3-(4-(tert-butyl)thiazol-2-yl)urea

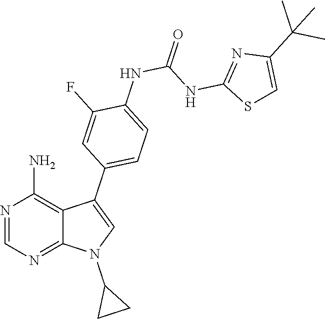 [SMILES: CC(C)(C)c1csc(NC(=O)Nc2ccc(-c3cn(C4CC4)c4ncnc(N)c34)cc2F)n1]

The title compound was obtained following the general procedure for urea formation (Method Ic-A), starting from phenyl (4-(4-amino-7-cyclopropyl-7H-pyrrolo[2,3-d]pyrimidin-5-yl)-2-fluorophenyl)carbamate (Ic-E11, 0.040 g, 0.099 mmol) and 4-(tert-butyl)thiazol-2-amine (0.015 g, 0.099 mmol), and was obtained as an off-white solid (7.0 mg, 15% yield). ¹H NMR (400 MHz, DMSO-d₆) δ=10.99 (bs, 1H), 9.29 (bs, 1H), 8.46 (s, 1H), 8.26-8.30 (m, 1H), 7.67 (s, 1H), 7.38-7.41 (m, 1H), 7.27-7.30 (m, 1H), 6.70 (s, 1H), 3.71 (bs, 1H), 1.27 (s, 9H), 1.10-1.10 (m, 4H). LCMS: 466.0 [M+H].

### Example IC-19

### 1-(4-(4-amino-7-cyclopropyl-7H-pyrrolo[2,3-d]pyrimidin-5-yl)-2-fluorophenyl)-3-(5-(tert-butyl)-1,3,4-thiadiazol-2-yl)urea

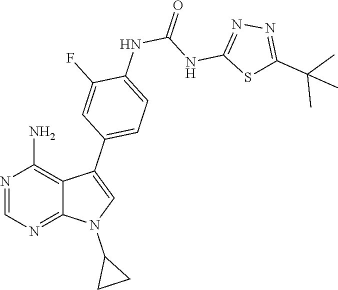 [SMILES: CC(C)(C)c1nnc(NC(=O)Nc2ccc(-c3cn(C4CC4)c4ncnc(N)c34)cc2F)s1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.050 g, 0.176 mmol) and phenyl (5-(tert-butyl)-1,3,4-thiadiazol-2-yl)carbamate (Ic-E12, 0.049 g, 0.176 mmol), and was obtained as an off-white solid (2.0 mg, 2% yield). ¹H NMR (400 MHz, CD₃OD) δ=8.19-8.23 (m, 2H), 7.30-7.35 (m, 2H), 7.23 (s, 1H), 3.50-3.54 (m, 1H), 1.49 (s, 9H), 1.08-1.17 (m, 4H). LCMS: 466.9 [M+H].

### Example IC-20

### 1-(4-(4-amino-7-cyclopropyl-7H-pyrrolo[2,3-d]pyrimidin-5-yl)-2-fluorophenyl)-3-(3-(tert-butyl)isothiazol-5-yl)urea

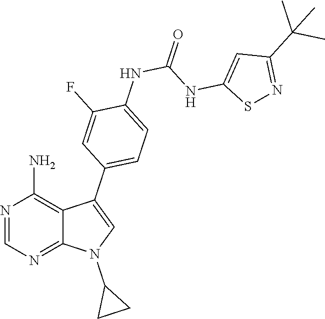 [SMILES: CC(C)(C)c1cc(NC(=O)Nc2ccc(-c3cn(C4CC4)c4ncnc(N)c34)cc2F)sn1]

The title compound was obtained following the general procedure for urea formation (Method Ic-A), starting from phenyl (4-(4-amino-7-cyclopropyl-7H-pyrrolo[2,3-d]pyrimidin-5-yl)-2-fluorophenyl)carbamate (Ic-E11, 0.040 g, 0.099 mmol) and 4-(tert-butyl)thiazol-2-amine (0.015 g, 0.099 mmol), and was obtained as an off-white solid (2.0 mg, 4% yield). ¹H NMR (400 MHz, CD₃OD) δ=8.21 (s, 1H), 8.11-8.17 (m, 1H), 7.30-7.34 (m, 2H), 7.23 (s, 1H), 6.78 (s, 1H), 3.50-3.54 (m, 1H), 1.35 (s, 9H), 1.08-1.30 (m, 4H). LCMS: 466.0 [M+H].

### Example IC-21

### 1-(4-(4-amino-7-cyclopropyl-7H-pyrrolo[2,3-d]pyrimidin-5-yl)-2-fluorophenyl)-3-(3-(tert-butyl)-1,2,4-thiadiazol-5-yl)urea

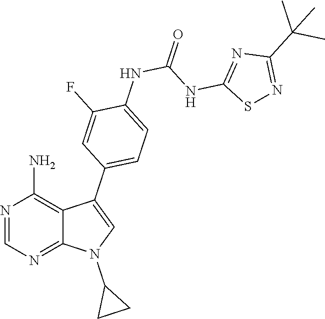 [SMILES: CC(C)(C)c1nsc(NC(=O)Nc2ccc(-c3cn(C4CC4)c4ncnc(N)c34)cc2F)n1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.050 g, 0.176 mmol) and phenyl (3-(tert-butyl)-1,2,4-thiadiazol-5-yl)carbamate (Ic-E13, 0.049 g, 0.176 mmol), and was obtained as an off-white solid (8.0 mg, 10% yield). ¹H NMR (400 MHz, DMSO-d₆) δ=11.54 (bs, 1H), 9.00 (bs, 1H), 8.09-8.18 (m, 2H), 7.28-7.39 (m, 3H), 6.19 (bs, 2H), 3.57-3.61 (m, 1H), 1.34 (s, 9H), 1.03-1.05 (m, 4H), LCMS: 467.0 [M+H].

### Example IC-22

### 1-(4-(4-amino-7-cyclopropyl-7H-pyrrolo[2,3-d]pyrimidin-5-yl)-2-fluorophenyl-3-(1-tert-butyl)-1H-1,2,4-Triazol-3-yl)urea

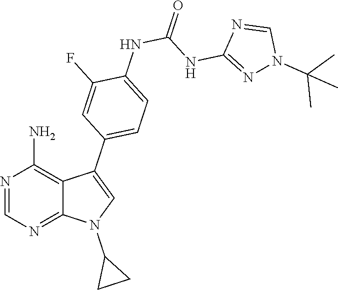 [SMILES: CC(C)(C)n1cnc(NC(=O)Nc2ccc(-c3cn(C4CC4)c4ncnc(N)c34)cc2F)n1]

The title compound was obtained following the general procedure for urea formation (Method Ic-A), starting from phenyl (4-(4-amino-7-cyclopropyl-7H-pyrrolo[2,3-d]pyrimidin-5-yl)-2-fluorophenyl)carbamate (Ic-E11, 0.040 g, 0.099 mmol) and 1-(tert-butyl)-li-1,2,4-triazol-3-amine (0.014 g, 0.099 mmol), and was obtained as an off-white solid (3.0 mg, 6% yield). ¹H NMR (400 MHz, DMSO-d₆) δ=10.70 (bs, 1H), 10.16 (bs, 1H), 8.54 (s, 1H), 8.31-8.35 (m, 1H), 8.16 (s, 1H), 7.25-7.37 (m, 3H), 6.17 (bs, 2H), 3.56-3.59 (m, 1H), 1.57 (s, 9H), 1.02-1.05 (m, 4H). LCMS: 450.0 [M+H].

### Example IC-23

### 1-(4-(4-amino-7-cyclopropyl-7H-pyrrolo[2,3-d]pyrimidin-5-yl)-2-fluorophenyl)-3-(5-(tert-butyl)-1,3,4-oxadiazol-2-yl)urea

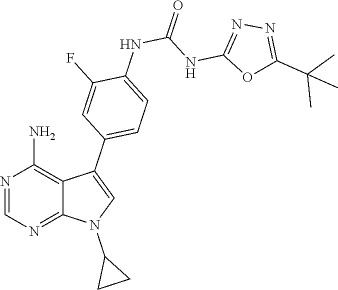 [SMILES: CC(C)(C)c1nnc(NC(=O)Nc2ccc(-c3cn(C4CC4)c4ncnc(N)c34)cc2F)o1]

The title compound was obtained following the general procedure for urea formation (Method Ic-A), starting from phenyl (4-(4-amino-7-cyclopropyl-7H-pyrrolo[2,3-d]pyrimidin-5-yl)-2-fluorophenyl)carbamate (Ic-E11, 0.040 g, 0.099 mmol) and 5-(tert-butyl)-1,3,4-oxadiazol-2-amine (0.014 g, 0.099 mmol), and was obtained as an off-white solid (2.0 mg, 4% yield). ¹H NMR (400 MHz, CD₃OD) δ=8.35 (s, 1H), 8.26-8.30 (m, 1H), 7.48 (s, 1H), 7.31-7.40 (m, 2H), 3.69-3.71 (m, 1H), 1.45 (s, 9H), 1.17-1.22 (m, 4H). LCMS: 451.0 [M+H].

### Example IC-24

### 1-(4-(4-amino-7-(pyridin-3-yl)-7H-pyrrolo[2,3-d]pyrimidin-5-yl)-2-fluorophenyl)-3-(3-(1-(trifluoromethyl)cyclopropyl)isoxazol-5-yl)urea

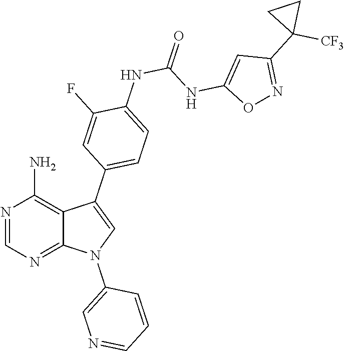 [SMILES: Nc1ncnc2c1c(-c1ccc(NC(=O)Nc3cc(C4(C(F)(F)F)CC4)no3)c(F)c1)cn2-c1cccnc1]

The title compound was obtained following the general procedure for urea formation (Method Ic-A), starting from 5-(4-amino-3-fluorophenyl)-7-(pyridin-3-yl)-7H-pyrrolo[2,3-d]pyrimidin-4-amine (Ic-D8, 0.100 g, 0.312 mmol) and phenyl (3-(1-(trifluoromethyl)cyclopropyl)isoxazol-5-yl)carbamate (Ic-E6, 0.107 g, 0.343 mmol), and was obtained as a white solid (0.023 g, 14% yield). ¹H NMR (400 MHz, DMSO-d₆) δ=10.69 (bs, 1H), 9.13 (bs, 1H), 8.97 (bs, 1H), 8.62 (d, J=4.8 Hz, 1H), 8.31-8.36 (m, 2H), 8.20-8.24 (m, 1H), 7.95 (s, 1H), 7.62-7.65 (m, 1H), 7.38-7.50 (m, 2H), 6.76 (bs, 2H), 6.21 (s, 1H), 1.39-1.47 (m, 4H). LCMS: 538.8 [M+H].

### Example IC-25

### 1-(4-(4-amino-7-(pyridin-4-yl)-7H-pyrrolo[2,3-d]pyrimidin-5-yl)-2-fluorophenyl)-3-(3-(1-(trifluoromethyl)cyclopropyl)isoxazol-5-yl)urea

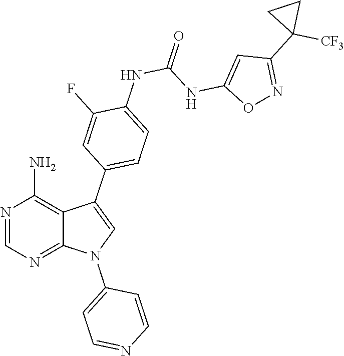 [SMILES: Nc1ncnc2c1c(-c1ccc(NC(=O)Nc3cc(C4(C(F)(F)F)CC4)no3)c(F)c1)cn2-c1ccncc1]

The title compound was obtained following the general procedure for urea formation (Method Ic-A), starting from 5-(4-amino-3-fluorophenyl)-7-(pyridin-4-yl)-71-pyrrolo[2,3-d]pyrimidin-4-amine (Ic-D9, 0.080 g, 0.250 mmol) and phenyl (3-(1-(trifluoromethyl)cyclopropyl)isoxazol-5-yl)carbamate (Ic-E6, 0.086 g, 0.275 mmol), and was obtained as an off-white solid (0.046 g, 33% yield). ¹H NMR (400 MHz, DMSO-d₆) δ=10 68 (bs, 1H), 9.00 (bs, 1H), 8.89 (d, J=6.4 Hz, 2H), 8.58 (d, J=6.4 Hz, 2H), 8.43 (s, 1H), 8.21-8.27 (m, 2H), 7.49-7.52 (m, 1H), 7.39-7.42 (m, 1H), 6.90 (bs, 2H), 6.22 (s, 1H), 1.39-1.49 (m, 4H). LCMS: 538.9 [M+H].

### Example IC-26

### 1-(4-(4-amino-7-(1-methylpiperidin-4-yl)-7H-pyrrolo[2,3-d]pyrimidin-5-yl)-2-fluorophenyl)-3-(3-(1-(trifluoromethyl)cyclopropyl)isoxazol-5-yl)urea

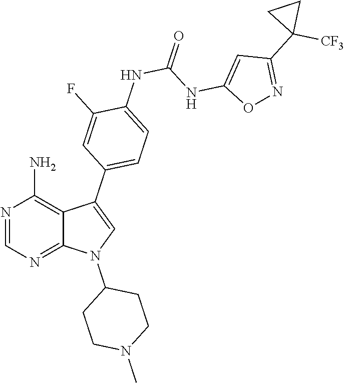 [SMILES: CN1CCC(n2cc(-c3ccc(NC(=O)Nc4cc(C5(C(F)(F)F)CC5)no4)c(F)c3)c3c(N)ncnc32)CC1]

The title compound was obtained following the general procedure for urea formation (Method Ic-A), starting from 5-(4-amino-3-fluorophenyl)-7-(1-methylpiperidin-4-yl)-7H-pyrrolo[2,3-d]pyrimidin-4-anine (Ic-D10, 0.020 g, 0.059 mmol) and phenyl (3-(1-(trifluoromethyl)cyclopropyl)isoxazol-5-yl)carbamate (Ic-E6, 0.018 g, 0.059 mmol), and was obtained as a white solid (2.4 mg, 7% yield). ¹H NMR (400 MHz, DMSO-d₆) δ=10.68 (bs, 1H), 8.96 (bs, 1H), 8.32 (s, 1H), 8.16-8.20 (m, 1H), 7.55 (s, 1H), 7.38-7.41 (m, 1H), 7.29-7.31 (m, 1H), 6.98 (bs, 2H), 6.21 (s, 1H), 4.85-4.91 (m, 1H), 3.59-3.62 (m, 4H), 2.85 (s, 3H), 2.21-2.34 (m, 4H), 1.38-1.49 (m, 4H). LCMS: 559.2 [M+H].

### Example IC-27

### 1-(4-(4-amino-7-cyclopropyl-7H-pyrrolo[2,3-d]pyrimidin-5-yl)-2-fluorophenyl)-(3-(3-methyloxetan-3-yl)isoxazol-5-yl)urea

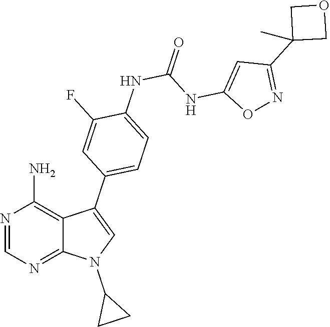 [SMILES: CC1(c2cc(NC(=O)Nc3ccc(-c4cn(C5CC5)c5ncnc(N)c45)cc3F)on2)COC1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.050 g, 0.176 mmol) and phenyl (3-(3-methyloxetan-3-yl)isoxazol-5-yl)carbamate (Ic-E14, 0.048 g, 0.176 mmol), and was obtained as a white solid (3.4 mg, 4% yield). ¹H NMR (400 MHz, DMSO-d₆) δ=10.56 (s, 1H), 8.94 (bs, 1H), 8.42 (s, 1H), 8.19 (t, J=8.4 Hz, 1H), 7.57 (bs, 3H), 7.38 (d, J=10.8 Hz, 1H), 7.28 (d, J=8.4 Hz, 1H), 6.22 (s, 1H), 4.77 (d, J=5.6 Hz, 2H), 4.52 (d, J=5.6 Hz, 2H), 3.68-3.69 (m, 1H), 1.63 (s, 3H), 1.09-1.10 (m, 4H). LCMS: 464.1 [M+H].

### Example IC-28

### 1-(4-(4-amino-7-cyclopropyl-7H-pyrrolo[2,3-d]pyrimidin-5-yl)-2-fluorophenyl)-3-(3-(trifluoromethyl)isoxazol-5-yl)urea

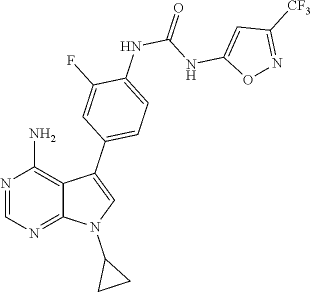 [SMILES: Nc1ncnc2c1c(-c1ccc(NC(=O)Nc3cc(C(F)(F)F)no3)c(F)c1)cn2C1CC1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.050 g, 0.176 mmol) and phenyl (3-(trifluoromethyl)isoxazol-5-yl)carbamate (Ic-E15, 0.048 g, 0.176 mmol), and was obtained as a white solid (2.9 mg, 4% yield). ¹H NMR (400 MHz, DMSO-d₆) δ=11.03 (bs, 1H), 8.97 (bs, 1H), 8.19 (s, 1H), 8.10 (t, J=8.4 Hz, 1H), 7.27-7.38 (m, 3H), 6.54 (s, 1H), 6.30 (bs, 2H), 3.56-3.60 (m, 1H), 1.03-1.05 (m, 4H) LCMS: 461.9 [M+H].

### Example IC-29

### 1-(4-(4-amino-7-cyclopropyl-7H-pyrrolo[2,3-d]pyrimidin-5-yl)-2-fluorophenyl)-3-(3-(1-hydroxy-2-methylpropan-2-yl)isoxazol-5-yl)urea

### Step 1: Synthesis of 1-(4-(4-amino-7-cyclopropyl-7H-pyrrolo[2,3-d]pyrimidin-5-yl)-2-fluorophenyl)-3-(3-(1-((tert-butyldimethylsilyl)oxy)-2-methylpropan-2-yl)isoxazol-5-yl)urea

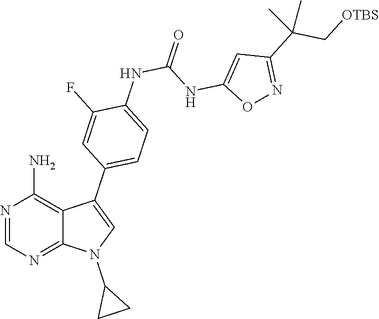 [SMILES: CC(C)(CO[Si](C)(C)C(C)(C)C)c1cc(NC(=O)Nc2ccc(-c3cn(C4CC4)c4ncnc(N)c34)cc2F)on1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.131 g, 0.461 mmol) and phenyl (3-(1-((tert-butyldimethylsilyl)oxy)-2-methylpropan-2-yl)isoxazol-5-yl)carbamate (Ic-E16, 0.180 g, 0.461 mmol), and was obtained as an off-white solid (0.013 g, 5% yield). LCMS: 580.0 [M+H].

### Step 2: Synthesis of 1-(4-(4-amino-7-cyclopropyl-?H-pyrrolo[2,3-d]pyrimidin-5-yl)-2-fluorophenyl)-3-(3-(1-hydroxy-2-methylpropan-2-yl)isoxazol-5-yl)urea

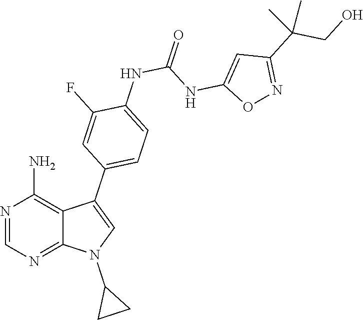 [SMILES: CC(C)(CO)c1cc(NC(=O)Nc2ccc(-c3cn(C4CC4)c4ncnc(N)c34)cc2F)on1]

TBAF (1M in THF, 0.067 ml) was added to a solution of 1-(4-(4-amino-7-cyclopropyl-7H-pyrrolo[2,3-d]pyrimidin-5-yl)-2-fluorophenyl)-3-(3-(1-((tert-butyldimethylsilyl)oxy)-2-methylpropan-2-yl)isoxazol-5-yl)urea (0.013 g, 0.022 mmol) in THF (2 ml) at 0° C. and the resulting solution was stirred at 25° C. for 4 h. Following completion of the reaction (as indicated by TLC), the reaction mixture was concentrated under reduced pressure to give crude material which was purified by preparative HPLC to afford the title compound as a white solid (TFA salt, 2.2 mg, 21% yield). ¹H NMR (400 MHz, CD₃OD) δ=8.36 (s, 1H), 8.23 (t, J=8.4 Hz, 1H), 7.49 (s, 1H), 7.30-7.38 (m, 2H), 6.20 (s, 1H), 3.69-3.72 (m, 1H), 3.61 (s, 2H), 1.32 (s, 6H), 1.18-1.24 (m, 4H). LCMS: 466.2 [M+H].

### Example IC-30

### 1-(4-(4-amino-7-cyclopropyl-7H-pyrrolo[2,3-d]pyrimidin-5-yl)-2-fluorophenyl)-3-(3-(SEC-butyl)isoxazol-5-yl)urea

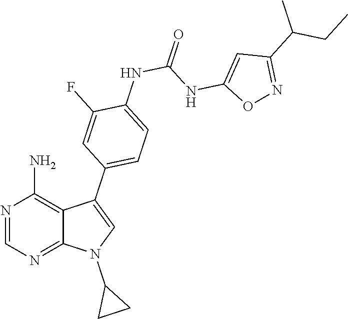 [SMILES: CCC(C)c1cc(NC(=O)Nc2ccc(-c3cn(C4CC4)c4ncnc(N)c34)cc2F)on1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.055 g, 0.194 mmol) and phenyl (3-(sec-butyl)isoxazol-5-yl)carbamate (Ic-E17, 0.051 g, 0.194 mmol), and was obtained as a white solid (0.012 g, 14% yield). ¹H NMR (400 MHz, DMSO-d₆) δ=10.39 (bs, 1H), 8.84 (bs, 1H), 8.12-8.14 (m, 2H), 7.25-7.36 (m, 3H), 6.16 (bs, 2H), 6.02 (s, 1H), 3.56-3.59 (m, 1H), 2.68-2.73 (m, 1H), 1.56-1.60 (m, 2H), 1.18-1.20 (m, 3H), 1.01-1.10 (m, 41), 0.81-0.89 (m, 3H). LCMS: 450.0 [M+H].

### Example IC-31

### 1-(4-(4-amino-7-cyclopropyl-7H-pyrrolo[2,3-d]pyrimidin-5-yl)-2-fluorophenyl)-3-(3-pentan-3-yl)isoxazol-5-yl)urea

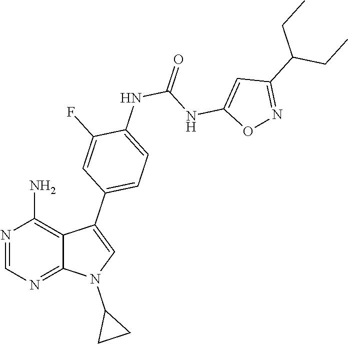 [SMILES: CCC(CC)c1cc(NC(=O)Nc2ccc(-c3cn(C4CC4)c4ncnc(N)c34)cc2F)on1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.050 g, 0.176 mmol) and phenyl (3-(pentan-3-yl)isoxazol-5-yl)carbamate (Ic-E18, 0.048 g, 0.176 mmol), and was obtained as an off-white solid (0.016 g, 19% yield). ¹H NMR (400 MHz, DMSO-d₆) δ=10.65 (bs, 1H), 9.06 (bs, 1H), 8.37 (s, 1H), 8.18 (t, J=8.4 Hz, 1H), 7.51 (s, 1H), 7.35-7.38 (m, 1H), 7.26-7.28 (m, 3H), 5.98 (s, 1H), 3.65-3.68 (m, 1H), 1.49-1.68 (m, 4H), 1.07-1.09 (m, 4H), 0.79-0.82 (m, 6H). LCMS: 464.0 [M+H].

### Example IC-32

### 1-(4-(4-amino-7-cyclopropyl-7H-pyrrolo[2,3-d]pyrimidin-5-yl)-2-fluorophenyl)-3-(3-isopropylisoxazol-5-yl)urea

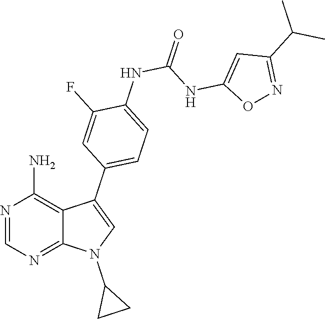 [SMILES: CC(C)c1cc(NC(=O)Nc2ccc(-c3cn(C4CC4)c4ncnc(N)c34)cc2F)on1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.056 g, 0.198 mmol) and phenyl (3-isopropylisoxazol-5-yl)carbamate (Ic-E19, 0.049 g, 0.198 mmol), and was obtained as an off-white solid (0.018 g, 21% yield). ¹H NMR (400 MHz, DMSO-<A) δ=10.34 (bs, 1H), 8.81 (bs, 1H), 8.12-8.16 (m, 2H), 7.25-7.36 (m, 3H), 6.15 (bs, 2H), 6.04 (s, 1H), 3.55-3.59 (m, 1H), 2.89-2.96 (m, 1H), 1.21-1.25 (m, 6H), 1.07-1.09 (m, 4H). LCMS: 436.0 [M+H].

### Example IC-33

### 1-(4-(4-amino-7-cyclopropyl-7H-pyrrolo[2,3-d]pyrimidin-5-yl)-2-fluorophenyl)-3-(3-ethylisoxazol-5-yl)urea

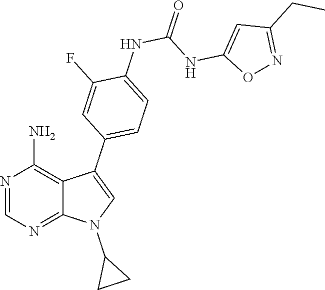 [SMILES: CCc1cc(NC(=O)Nc2ccc(-c3cn(C4CC4)c4ncnc(N)c34)cc2F)on1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.061 g, 0.215 mmol) and phenyl (3-ethylisoxazole-5-yl)carbamate (Ic-E20, 0.050 g, 0.215 mmol), and was obtained as an off-white solid (0.020 g, 22% yield). ¹H NMR (400 MHz, DMSO-d₆) δ=10 31 (bs, 1H), 8.81 (bs, 1H), 8.12-8.17 (m, 2H), 7.25-7.36 (m, 3H), 6.15 (bs, 2H), 6.03 (s, 1H), 3.55-3.60 (m, 1H), 2.54-2.60 (m, 2H), 1.05-1.21 (m, 3H), 1.03-1.04 (m, 4H). LCMS: 422.0 [M+H].

### Example IC-34

### 1-(4-(4-amino-7-cyclopropyl-7H-pyrrolo[2,3-d]pyrimidin-5-yl)-2-fluorophenyl)-3-(3-(1-methylcyclobutyl)isoxazol-5-yl)urea

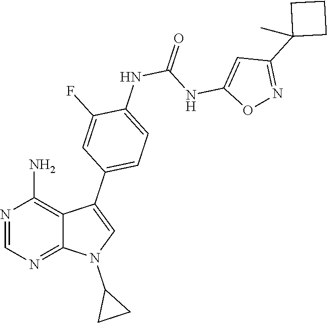 [SMILES: CC1(c2cc(NC(=O)Nc3ccc(-c4cn(C5CC5)c5ncnc(N)c45)cc3F)on2)CCC1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.075 g, 0.265 mmol) and phenyl (3-(1-methylcyclobutyl)isoxazol-5-yl)carbamate (Ic-E21, 0.072 g, 0.265 mmol), and was obtained as a white solid (6.8 mg, 5% yield). ¹H NMR (400 MHz, DMSO-d₆) δ=10.42 (bs, 1H), 8.90 (bs, 1H), 8.42 (s, 1H), 8.18-8.22 (m, 1H), 7.58 (s, 1H), 7.37-7.40 (m, 1H), 7.27-7.29 (m, 1H), 6.06 (s, 1H), 3.67-3.71 (m, 1H), 2.34-2.38 (m, 2H), 1.85-2.08 (m, 4H), 1.42 (s, 3H), 1.07-1.11 (m, 4H). LCMS: 462.2 [M+H].

### Example IC-35

### 1-(4-(4-amino-7-cyclopropyl-7H-pyrrolo[2,3-d]pyrimidin-5-yl)-2-fluorophenyl)-3-(3-(2-cyanopropan-2-yl)isoxazol-5-yl)urea

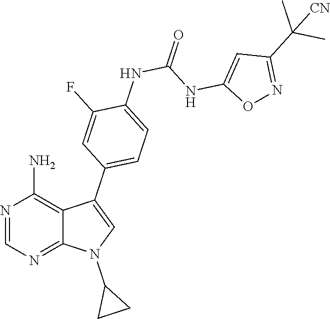 [SMILES: CC(C)(C#N)c1cc(NC(=O)Nc2ccc(-c3cn(C4CC4)c4ncnc(N)c34)cc2F)on1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.070 g, 0.247 mmol) and phenyl (3-(2-cyanopropan-2-yl)isoxazol-5-yl)carbamate (Ic-E22, 0.067 g, 0.247 mmol), and was obtained as a white solid (4.1 mg, 3% yield). ¹H NMR (400 MHz, CD₃OD)) δ=8.33 (s, 1H), 8.20-8.24 (m, 1H), 7.43 (s, 1H), 7.31-7.38 (m, 2H), 6.33 (s, 1H), 3.50-3.66 (m, 1H), 1.77 (s, 6H), 1.15-1.21 (m, 1H). LCMS: 461.1 [M+H].

### Example IC-36

### 1-(4-(4-amino-7-cyclopropyl-7H-pyrrolo[2,3-d])pyrimidin-5-yl)-2-fluorophenyl)-3-(3-(hydroxymethyl)isoxazol-5-yl)urea

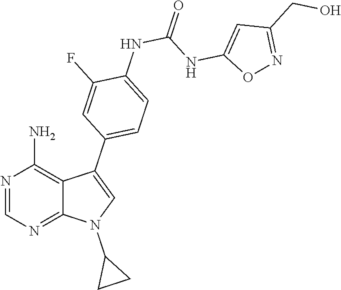 [SMILES: Nc1ncnc2c1c(-c1ccc(NC(=O)Nc3cc(CO)no3)c(F)c1)cn2C1CC1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.250 g, 0.882 mmol) and phenyl (3-((tert-butyldimethylsilyl)oxy)methyl)isoxazol-5-yl)carbamate (Ic-E23, 0.308 g, 0.882 mmol), and was obtained as an off-white solid (0.029 g, 8% yield). ¹H NMR (400 MHz, DMSO-d₆) δ=10.43 (bs, 1H), 8.92 (bs, 1H), 8.40 (s, 1H), 8.18-8.22 (m, 1H), 7.56 (s, 1H), 7.37-7.40 (m, 1H), 7.27-7.29 (m, 1H), 6.12 (s, 1H), 4.43 (s, 2H), 3.66-3.71 (m, 1H), 1.08-1.11 (m, 4H). LCMS: 422.0 [M−H].

Note: cleavage of the TBDMS group was observed during purification when eluting with a gradient of 10 mM ammonium acetate in water and ACN.

### Example IC-37

### 1-(5-(4-amino-7-cyclopropyl-7H-pyrrolo[2,3-d]pyrimidin-5-yl)pyridin-2-yl)-3-(3-(1-(trifluoromethyl)cyclopropyl)isoxazol-5-yl)urea

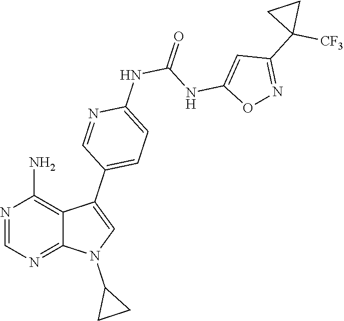 [SMILES: Nc1ncnc2c1c(-c1ccc(NC(=O)Nc3cc(C4(C(F)(F)F)CC4)no3)nc1)cn2C1CC1]

The title compound was obtained following the general procedure for urea formation (Method Ic-A), starting from 5-(6-aminopyridin-3-yl)-7-cyclopropyl-7H-pyrrolo[2,3-d]pyrimidin-4-anine (Ic-D6, 0.300 g, 1.127 mmol) and phenyl (3-(1-(trifluoromethyl)cyclopropyl)isoxazol-5-yl)carbamate (Ic-E6, 0.352 g, 1.127 mmol), and was obtained as an off-white solid (0.018 g, 3% yield). ¹H NMR (400 MHz, DMSO-d₆) δ=11.77 (bs, 1H), 9.85 (bs, 1H), 8.39-8.40 (m, 11H), 8.18 (s, 11H), 7.86-7.88 (m, 1H), 7.62 (d, J=8.4 Hz, 1H), 7.34 (s, 1H), 6.26 (s, 1H), 6.23 (bs, 2H), 3.58-3.61 (m, 1H), 1.40-1.49 (m, 4H), 1.03-1.06 (m, 4H). LCMS: 485.0 [M+H].

### Example IC-38

### 1-(4-(4-amino-7-cyclopropyl-7H-pyrrolo[2,3-d]pyrimidin-5-yl)-2-methylphenyl)-3-(3-(1-(trifluoromethyl)cyclopropyl)isoxazol-5-yl)urea

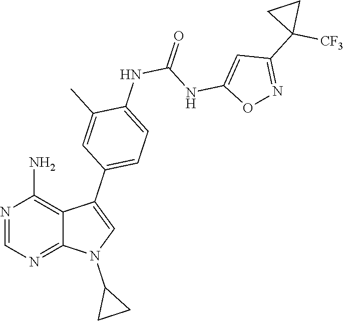 [SMILES: Cc1cc(-c2cn(C3CC3)c3ncnc(N)c23)ccc1NC(=O)Nc1cc(C2(C(F)(F)F)CC2)no1]

The title compound was obtained following the general procedure for urea formation (Method Ic-A), starting from 5-(4-amino-3-methylphenyl)-7-cyclopropyl-7H-pyrrolo[2,3-d]pyrimidin-4-amine (Ic-D11, 0.270 g, 0.967 mmol) and phenyl (3-(1-(trifluoromethyl)cyclopropyl)isoxazol-5-yl)carbamate (Ic-E6, 0.302 g, 0.967 mmol), and was obtained as an off-white solid (0.080 g, 16% yield). ¹H NMR (400 MHz, DMSO-d₆) δ=10.63 (bs, 1H), 8.29 (bs, 1H), 8.16 (s, 1H), 7.87 (d, J=8.4 Hz, 1H), 7.22-7.32 (m, 3H), 6.17 (s, 1H), 6.08 (bs, 2H), 3.55-3.60 (m, 1H), 2.29 (s, 3H), 1.37-1.48 (m, 4H), 1.00-1.05 (m, 4H). LCMS: 498.1 [M+H].

### Example IC-39

### 1-(4-(4-amino-7-(3-hydroxycyclobutyl)-7H-pyrrolo[2,3-d]pyrimidin-5-yl)-2-fluorophenyl)-3-(3-(1-(trifluoromethyl)cyclopropyl)isoxazol-5-yl)urea

### Step 1: Synthesis of 1-(4-(4-amino-7-(3-(benzyloxy)cyclobutyl)-7H-pyrrolo[2,3-d]pyrimidin-5-yl)-2-fluorophenyl)-3-(3-(1-(trifluoromethyl)cyclopropyl)isoxazol-5-yl)urea

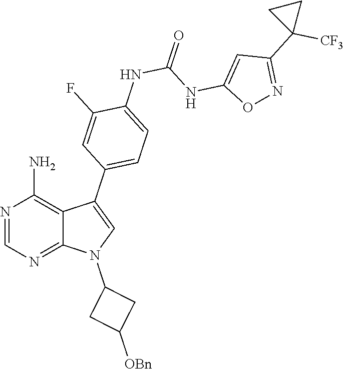 [SMILES: Nc1ncnc2c1c(-c1ccc(NC(=O)Nc3cc(C4(C(F)(F)F)CC4)no3)c(F)c1)cn2C1CC(OCc2ccccc2)C1]

The title compound was obtained following the general procedure for urea formation (Method Ic-B), starting from 5-(4-amino-3-fluorophenyl)-7-(3-(benzyloxy)cyclobutyl)-7H-pyrrolo[2,3-d]pyrimidin-4-amine (Ic-D12, 0.033 g, 0.082 mmol) and phenyl (3-(1-(trifluoromethyl)cyclopropyl)isoxazol-5-yl)carbamate (Ic-E6, 0.028 g, 0.090 mmol), and was obtained as a yellow gum (0.038 g, 29% yield). LCMS: 622.3 [M+H].

### Step 2: Synthesis of 1-(4-(4-amino-7-(3-hydroxycyclobutyl)-7H-pyrrolo[2,3-d]pyrimidin-5-yl)-2-fluorophenyl)-3-(3-(1-(trifluoromethyl)cyclopropyl)isoxazol-5-yl)urea

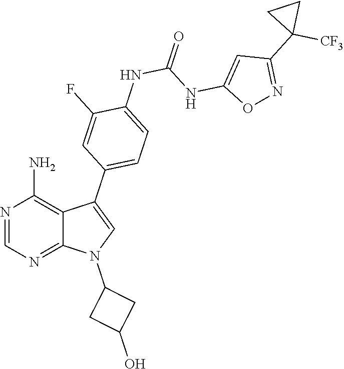 [SMILES: Nc1ncnc2c1c(-c1ccc(NC(=O)Nc3cc(C4(C(F)(F)F)CC4)no3)c(F)c1)cn2C1CC(O)C1]

Boron trichloride (1M in DCM, 0.901 mL, 0.901 mmol) was added dropwise to a solution of 1-(4-(4-amino-7-(3-(benzyloxy)cyclobutyl)-7H-pyrrolo[2,3-d]pyrimidin-5-yl)-2-fluorophenyl)-3-(3-(1-(trifluoromethyl)cyclopropyl)isoxazol-5-yl)urea (0.070 g, 0.113 mmol) in DCM (5 mL) at −60° C. and the resulting mixture was stirred at 0° C. for 3 h. Following completion of the reaction (as indicated by TLC and LCMS), the reaction mixture was cooled to −70° C., neutralized with NH₄OH (25% in water), and extracted with DCM (2×10 mL). The combined organic extracts were dried over Na₂SO₄, filtered, and concentrated under reduced pressure to yield crude material which was purified by preparative HPLC (eluting with a gradient of 1% TFA in water and ACN), affording the title product as a white solid (0.012 g, 20% yield). ¹H NMR (400 MHz, CD₃OD) δ=8.33 (s, 1H), 8.22-8.26 (m, 1H), 7.77 (s, 1H), 7.34-7.42 (m, 2H), 6.33 (s, 1H), 5.58-5.62 (m, 1H), 4.64-4.66 (m, 1H), 2.85-2.92 (m, 2H), 2.58-2.64 (m, 2H), 1.39-1.49 (m, 4H). LCMS: 531.8 [M+H].

### Example IC-40

### 1-(6-(4-amino-7-cyclopropyl-7H-pyrrolo[2,3-d]pyrimidin-5-yl)-4-methylpyridin-3-yl)-3-(3-(1-(trifluoromethyl)cyclopropyl)isoxazol-5-yl)urea

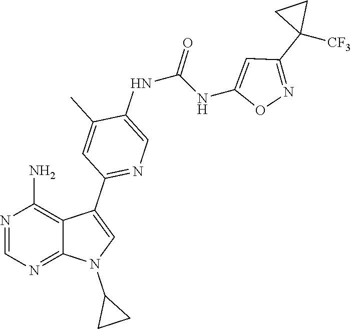 [SMILES: Cc1cc(-c2cn(C3CC3)c3ncnc(N)c23)ncc1NC(=O)Nc1cc(C2(C(F)(F)F)CC2)no1]

The title compound was obtained following the general procedure for urea formation (Method Ic-A), starting from 5-(5-amino-4-methylpyridin-2-yl)-7-cyclopropyl-7H-pyrrolo[2,3-d]pyrimidin-4-amine (Ic-D13, 0.050 g, 0.102 mmol) and phenyl (3-(1-(trifluoromethyl)cyclopropyl)isoxazol-5-yl)carbamate (Ic-E6, 0.032 g, 0.102 mmol), and was obtained as an off-white solid (8 mg, 16% yield). ¹H NMR (400 MHz, DMSO-d₆) δ=9.81 (bs, 1H), 8.76 (s, 1H), 8.51 (bs, 1H), 8.08 (s, 1H), 7.93-7.94 (m, 2H), 7.12 (bs, 2H), 6.17 (s, 1H), 3.53-3.56 (m, 1H), 2.30 (s, 3H), 1.37-1.46 (m, 4H), 1.05-1.08 (m, 4H). LCMS: 499.2 [M+H].

### Example IC-41

### 1-(4-(4-amino-7-cyclopropyl-7H-pyrrolo[2,3-d]pyrimidin-5-yl)-2,6-difluorophenyl)-3-(3-(1-(trifluoromethyl)cyclopropyl)isoxazol-5-yl)urea

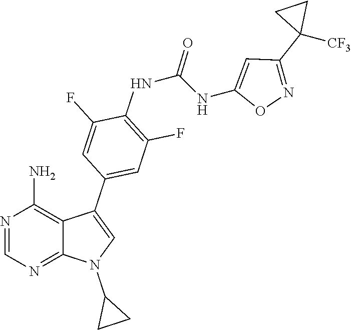 [SMILES: Nc1ncnc2c1c(-c1cc(F)c(NC(=O)Nc3cc(C4(C(F)(F)F)CC4)no3)c(F)c1)cn2C1CC1]

The title compound was obtained following the general procedure for urea formation (Method Ic-B), starting from 5-(4-amino-3,5-difluorophenyl)-7-cyclopropyl-7H-pyrrolo[2,3-d]pyrimidin-4-amine (Ic-D15, 0.080 g, 0.266 mmol) and phenyl (3-(1-(trifluoromethyl)cyclopropyl)isoxazol-5-yl)carbamate (Ic-E6, 0.083 g, 0.266 mmol), and was obtained as an off-white solid (8 mg, 5.6% yield). ¹H NMR (400 MHz, DMSO-d₄) δ=10.78 (bs, 1H), 8.71 (bs, 1H), 8.42 (bs, 1H), 7.67 (s, 1H), 7.56 (bs, 2H), 7.23-7.29 (m, 2H), 6.14 (s, 1H), 3.67-3.76 (m, 1H), 1.37-1.47 (m, 4H), 1.09-1.12 (m, 4H). LCMS: 519.7 [M+H].

### Example IC-42

### 1-(4-(4-amino-7-cyclopropyl-7H-pyrrolo[2,3-d]pyrimidin-5-yl)-2,5-difluorophenyl)-3-(3-(1-(trifluoromethyl)cyclopropyl)isoxazol-5-yl)urea

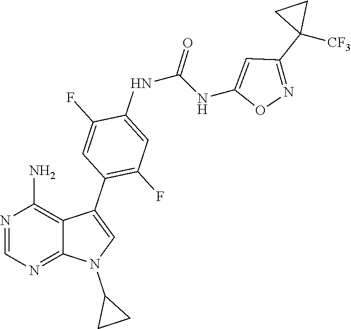 [SMILES: Nc1ncnc2c1c(-c1cc(F)c(NC(=O)Nc3cc(C4(C(F)(F)F)CC4)no3)cc1F)cn2C1CC1]

The title compound was obtained following the general procedure for urea formation (Method Ic-B), starting from 5-(4-amino-2,5-difluorophenyl)-7-cyclopropyl-7H-pyrrolo[2,3-d]pyrimidin-4-amine (Ic-D16, 0.100 g, 0.332 mmol) and phenyl (3-(1-(trifluoromethyl)cyclopropyl)isoxazol-5-yl)carbamate (Ic-E6, 0.104 g, 0.332 mmol), and was obtained as an off-white solid (8 mg, 5% yield). ¹H NMR (400 MHz, DMSO-d) δ=10.63 (bs, 1H), 9.01 (bs, 1H), 8.16 (s, 1H), 8.04-8.09 (m, 1H), 7.28-7.34 (m, 2H), 6.21 (s, 1H), 6.16 (bs, 2H), 3.55-3.61 (m, 1H), 1.36-1.49 (m, 4H), 1.00-1.04 (m, 4H). LCMS: 520.1 [M+H].

### Example IC-43

### 1-(4-(4-amino-7-cyclopropyl-7H-pyrrolo[2,3-d]pyrimidin-5-yl)-3,5-difluorophenyl)-3-(3-(1-(trifluoromethyl)cyclopropyl)isoxazol-5-yl)urea

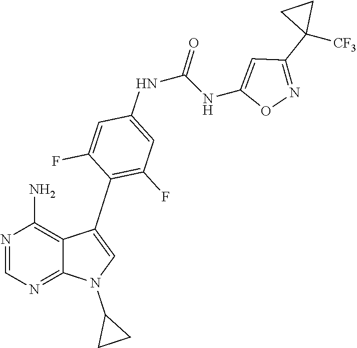 [SMILES: Nc1ncnc2c1c(-c1c(F)cc(NC(=O)Nc3cc(C4(C(F)(F)F)CC4)no3)cc1F)cn2C1CC1]

The title compound was obtained following the general procedure for urea formation (Method Ic-B), starting from 5-(4-amino-2,6-difluorophenyl)-7-cyclopropyl-7H-pyrrolo[2,3-d]pyrimidin-4-amine (Ic-D17, 0.0724 g, 0.240 mmol) and phenyl (3-(1-(trifluoromethyl)cyclopropyl)isoxazol-5-yl)carbamate (Ic-E6, 0.075 g, 0.240 mmol), and was obtained as an off-white solid (3 mg, 2% yield). ¹H NMR (400 MHz, DMSO-d₆) δ=8.14 (s, 1H), 7.39-7.42 (m, 2H), 7.24 (s, 1H), 6.15 (s, 1H), 6.02 (bs, 2H), 3.58-3.59 (m, 1H), 1.36-1.44 (m, 4H), 1.03-1.05 (m, 4H). LCMS: 520.2 [M+H].

### Example IC-44

### 1-(4-(4-amino-7-cyclopropyl-7H-pyrrolo[2,3-d]pyrimidin-5-yl)-3,5-difluorophenyl)-3-(3-(1-(trifluoromethyl)cyclopropyl)isoxazol-5-yl)urea

### Step 1: Synthesis of 1-(5-bromo-3-fluoropyridin-2-yl)-3-(3-(1-(trifluoromethyl)cyclopropyl)isoxazol-5-yl)urea

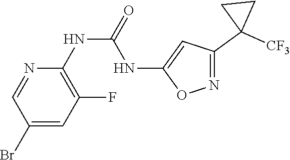 [SMILES: O=C(Nc1cc(C2(C(F)(F)F)CC2)no1)Nc1ncc(Br)cc1F]

The title compound was obtained following the general procedure for urea formation (Method Ic-B), starting from 5-bromo-3-fluoropyridin-2-amine (0.040 g, 0.209 mmol) and phenyl (3-(1-(trifluoromethyl)cyclopropyl)isoxazol-5-yl)carbamate (Ic-E6, 0.065 g, 0.209 mmol), and was obtained as a pale yellow solid (0.020 g, 23% yield). LCMS: 410.9 [M+H].

### Step 2: Synthesis of 1-(4-(4-amino-7-cyclopropyl-?1-pyrrolo[2,3-d]pyrimidin-5-yl)-3,5-difluorophenyl)-3-(3-(1-(triiodomethyl)cyclopropyl)isoxazol-5-yl)urea

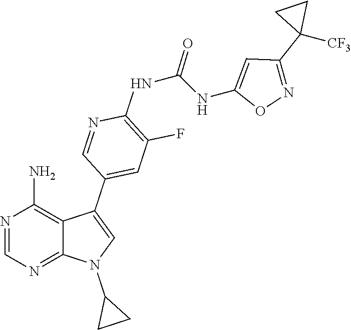 [SMILES: Nc1ncnc2c1c(-c1cnc(NC(=O)Nc3cc(C4(C(F)(F)F)CC4)no3)c(F)c1)cn2C1CC1]

The title compound was obtained by following a similar procedure described for Intermediate Ic-D5, starting from 1-(5-bromo-3-fluoropyridin-2-yl)-3-(3-(1-(trifluoromethyl)cyclopropyl)isoxazol-5-yl)urea (0.020 g, 0.049 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (prepared as reported in PCT Pub. No. WO 2018/015879, 0.024 g, 0.049 mmol), and was obtained as an off-white solid (2 mg, 7% yield). ¹H NMR (400 MHz, CD₃OD) δ=8.32-8.33 (m, 2H), 7.81-7.84 (m, 1H), 7.50 (s, 1H), 6.42 (s, 1H), 3.60-3.68 (m, 1H), 1.41-1.50 (m, 4H), 1.13-1.23 (m, 4H). LCMS: 503.1 [M+H]. Note: cleavage of the Boc groups was observed during the reaction.

### Example IC-45

### 1-(4-(4-amino-7-cyclopropyl-7H-pyrrolo[2,3-d]pyrimidin-5-yl)-2-fluorophenyl)-3-(3-(1-(trifluoromethyl)cyclobutyl)isoxazol-5-yl)urea

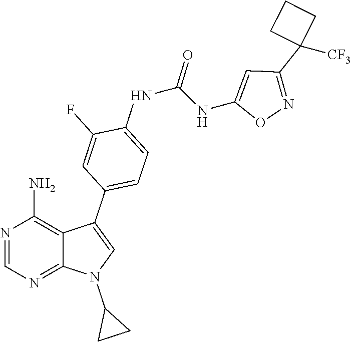 [SMILES: Nc1ncnc2c1c(-c1ccc(NC(=O)Nc3cc(C4(C(F)(F)F)CCC4)no3)c(F)c1)cn2C1CC1]

The title compound was obtained following the general procedure for urea formation (Method Ic-A), starting from 5-(4-amino-3-fluorophenyl)-7-cyclopropyl-7H-pyrrolo[2,3-<]pyrimidin-4-anine (Ic-D1, 0.080 g, 0.282 mmol) and phenyl (3-(1-(trifluoromethyl)cyclobutyl)isoxazol-5-yl)carbamate (Ic-E24, 0.092 g, 0.282 mmol), and was obtained as an off-white solid (0.035 g, 22% yield). ¹H NMR (400 MHz, DMSO-d₆) δ=10.66 (bs, 1H), 8.90 (bs, 1H), 8.21 (s, 1H), 8.12-8.16 (m, 1H), 7.26-7.36 (m, 3H), 6.41 (bs, 2H), 6.14 (s, 1H), 3.54-3.56 (m, 1H), 2.57-2.68 (m, 4H), 2.03-2.05 (m, 2H), 1.04-1.06 (m, 4H). LCMS: 515.9 [M+H].

### Example IC-46

### 1-(5-(4-amino-7-(2-hydroxy-2-methylpropyl)-7H-pyrrolo[2,3-d]pyrimidin-5-yl)pyridin-2-yl)-3-(3-(1-(trifluoromethyl)cyclopropyl)isoxazol-5-yl)urea

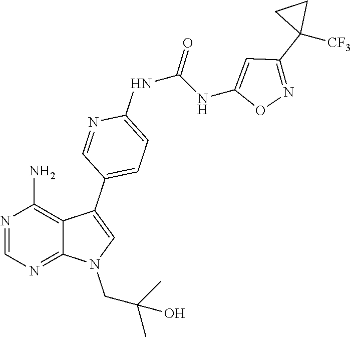 [SMILES: CC(C)(O)Cn1cc(-c2ccc(NC(=O)Nc3cc(C4(C(F)(F)F)CC4)no3)nc2)c2c(N)ncnc21]

The title compound was obtained following the general procedure for urea formation (Method Ic-B), starting from 1-(4-amino-5-(6-aminopyridin-3-yl)-7H-pyrrolo[2,3-d]pyrimidin-7-yl)-2-methylpropan-2-ol (Ic-D18, 0.100 g, 0.335 mmol) and phenyl (3-(1-(trifluoromethyl)cyclopropyl)isoxazol-5-yl)carbamate (Ic-E6, 0.105 g, 0.335 mmol), and was obtained as an off-white solid (0.010 g, 6% yield). ¹H NMR (400 MHz, DMSO-d₆) δ=11.74 (bs, 1H), 9.91 (bs, 1H), 8.41-8.43 (m, 2H), 7.89-7.92 (m, 1H), 7.80 (bs, 2H), 7.68-7.71 (m, 1H), 7.59 (s, 1H), 6.26 (s, 1H), 4.20 (s, 2H), 1.38-1.50 (m, 4H), 1.11-1.12 (m, 6H). LCMS: 516.9 [M+H].

### Example IC-47

### 1-(4-(4-amino-7-(2-hydroxy-2-methyl propyl)-7H-pyrrolo[2,3-d]pyrimidin-5-yl)-2-fluorophenyl)-3-(5-(1-(trifluoromethyl)cyclopropyl)isoxazol-3-yl)urea

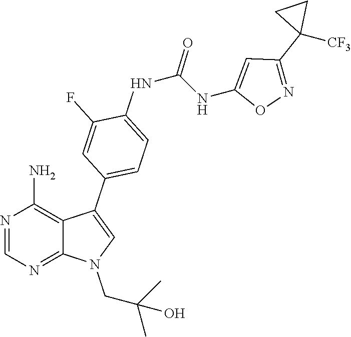 [SMILES: CC(C)(O)Cn1cc(-c2ccc(NC(=O)Nc3cc(C4(C(F)(F)F)CC4)no3)c(F)c2)c2c(N)ncnc21]

The title compound was obtained following the general procedure for urea formation (Method Ic-B), starting from 1-(4-amino-5-(4-amino-3-fluorophenyl)-7H-pyrrolo[2,3-d]pyrimidin-7-yl)-2-methylpropan-2-ol (Ic-D3, 0.120 g, 0.381 mmol) and phenyl (5-(1-(trifluoromethyl)cyclopropyl)isoxazol-3-yl)carbamate (Ic-E7, 0.119 g, 0.381 mmol), and was obtained as an off-white solid (0.028 g, 13% yield). ¹H NMR (400 MHz, DMSO-d₆) δ=10.04 (bs, 1H), 8.94 (bs, 1H), 8.41 (s, 1H), 8.23-8.27 (m, 1H), 7.55 (s, 1H), 7.36-7.40 (m, 1H), 7.27-7.30 (m, 1H), 6.91 (s, 1H), 4.18 (s, 2H), 1.51-1.58 (m, 4H), 1.07-1.11 (m, 6H). LCMS: 5342 [M+H].

### Example IC-48

### 1-(5-(4-amino-7-cyclopropyl-7H-pyrrolo[2,3-d]pyrimidin-5-yl)pyrimidin-2-yl)-3-(3-(1-(trifluoromethyl)cyclopropyl)isoxazol-5-yl)urea

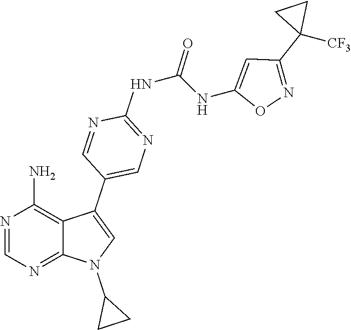 [SMILES: Nc1ncnc2c1c(-c1cnc(NC(=O)Nc3cc(C4(C(F)(F)F)CC4)no3)nc1)cn2C1CC1]

The title compound was obtained following the general procedure for urea formation (Method Ic-B), starting from 5-(2-aminopyrimidin-5-yl)-7-cyclopropyl-7H-pyrrolo[2,3-d]pyrimidin-4-amine (Ic-D19, 0.080 g, 0.299 mmol) and phenyl (3-(1-(trifluoromethyl)cyclopropyl)isoxazol-5-yl)carbamate (Ic-E6, 0.093 g, 0.299 mmol), and was obtained as an off-white solid (4 mg, 3% yield). ¹H NMR (400 MHz, DMSO-d₆) δ=10.76 (bs, 1H), 9.04 (bs, 1H), 8.65 (bs, 1H), 8.34 (bs, 2H), 7.61 (s, 1H), 6.66 (bs, 2H), 6.10 (s, 1H), 3.66-3.71 (m, 1H), 1.37-1.45 (m, 4H), 1.11-1.11 (m, 4H). LCMS: 485.9 [M+H].

### Example IC-49

### 1-(4-(4-amino-7-cyclopropyl-7H-pyrrolo[2,3-d]pyrimidin-5-yl)-2-(hydroxymethyl)phenyl)-3-(3-(1-(trifluoromethyl)cyclopropyl)isoxazol-5-yl)urea

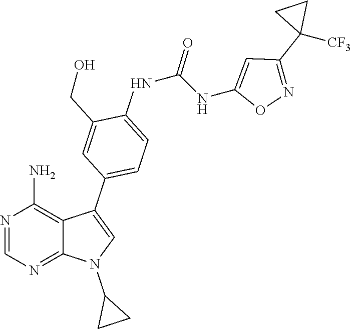 [SMILES: Nc1ncnc2c1c(-c1ccc(NC(=O)Nc3cc(C4(C(F)(F)F)CC4)no3)c(CO)c1)cn2C1CC1]

The title compound was obtained following the general procedure for urea formation (Method Ic-A), starting from (2-amino-5-(4-amino-7-cyclopropyl-7H-pyrrolo[2,3-d]pyrimidin-5-yl)phenyl)methanol (Ic-D20, 0.020 g, 0.068 mmol) and phenyl (3-(1-(trifluoromethyl)cyclopropyl)isoxazol-5-yl)carbamate (Ic-E6, 0.021 g, 0.068 mmol), and was obtained as an off-white solid (3 mg, 8% yield). ¹H NMR (400 MHz, DMSO-d₆) δ=10.96 (bs, 1H), 8.56 (bs, 1H), 8.17 (s, 1H), 7.91-7.93 (m, 1H), 7.34-7.42 (m, 2H), 7.21 (s, 1H), 6.04-6.17 (m, 3H), 5.49 (bs, 1H), 4.57 (bs, 2H), 3.56-3.61 (m, 1H), 1.37-1.47 (m, 4H), 1.00-1.06 (m, 4H). LCMS: 514.1 [M+H].

### Example IC-50

### 1-(4-(4-amino-7-cyclopropyl-7H-pyrrolo[2,3-d]pyrimidin-5-yl)-2-cyanophenyl)-3-(3-(1-(trifluoromethyl)cyclopropyl)isoxazol-5-yl)urea

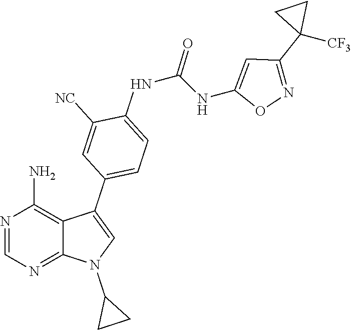 [SMILES: N#Cc1cc(-c2cn(C3CC3)c3ncnc(N)c23)ccc1NC(=O)Nc1cc(C2(C(F)(F)F)CC2)no1]

The title compound was obtained following the general procedure for urea formation (Method Ic-B), starting from 2-amino-5-(4-amino-7-cyclopropyl-7H-pyrrolo[2,3-d]pyrimidin-5-yl)benzonitrile (Ic-D21, 0.130 g, 0.287 mmol) and phenyl (3-(1-(trifluoromethyl)cyclopropyl)isoxazol-5-yl)carbamate (Ic-E6, 0.089 g, 0.287 mmol), and was obtained as an off-white solid (5 mg, 3% yield). ¹H NMR (400 MHz, DMSO-d₆) δ=8.62 (s, 1H), 7.43-7.52 (m, 3H), 6.86 (s, 1H), 6.05 (s, 1H), 3.67-3.70 (m, 1H), 1.38-1.46 (m, 4H), 1.08-1.10 (m, 4H). LCMS: 509.2 [M+H].

### Example IC-51

### 1-(4-(4-amino-7-(2-methoxyethyl)-7H)-pyrrolo[2,3-d]pyrimidin-5-yl)-2-fluorophenyl)-3-(3-(1-(trifluoromethyl cyclopropyl isoxazol-5-yl)urea

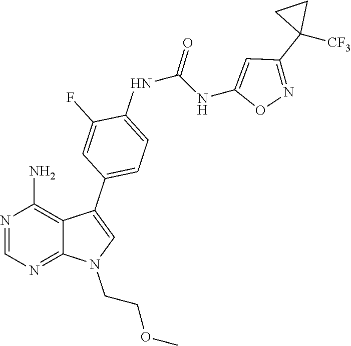 [SMILES: COCCn1cc(-c2ccc(NC(=O)Nc3cc(C4(C(F)(F)F)CC4)no3)c(F)c2)c2c(N)ncnc21]

The title compound was obtained following the general procedure for urea formation (Method Ic-B), starting from 5-(4-amino-3-fluorophenyl)-7-(2-methoxyethyl)-7H-pyrrolo[2,3-d]pyrimidin-4-amine (Ic-D22, 0.075 g, 0.249 mmol) and phenyl (3-(1-(trifluoromethyl)cyclopropyl)isoxazol-5-yl)carbamate (Ic-E6, 0.078 g, 0.249 mmol), and was obtained as an off-white solid (0.037 g, 28% yield). ¹H NMR (400 MHz, DMSO-d₆) δ=10.63 (bs, 1H), 8.88 (bs, 1H), 8.13-8.18 (m, 2H), 7.42 (s, 1H), 7.26-7.36 (m, 2H), 6.35 (bs, 2H), 6.20 (s, 1H), 4.34 (t, J=5.6 Hz, 2H), 3.72 (t, J=5.2 Hz, 2H), 3.26 (s, 3H), 1.38-1.49 (m, 4H). LCMS: 520.2 [M+H].

### Example IC-52

### 1-(4-(4-amino-7-(2-hydroxyethyl)-7H-pyrrolo[2,3-d]pyrimidin-5-yl)-2-fluorophenyl)-3-(3-(1-(trifluoromethyl)cyclopropyl)isoxazol-5-yl)urea

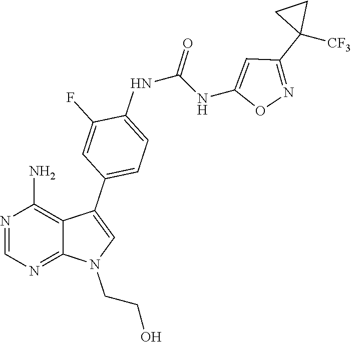 [SMILES: Nc1ncnc2c1c(-c1ccc(NC(=O)Nc3cc(C4(C(F)(F)F)CC4)no3)c(F)c1)cn2CCO]

The title compound was obtained following the general procedure for urea formation (Method Ic-B), starting from 2-(4-amino-5-(4-amino-3-fluorophenyl)-7H-pyrrolo[2,3-d]pyrimidin-7-yl)ethan-1-ol (Ic-D23, 0.080 g, 0.278 mmol) and phenyl (3-(1-(trifluoromethyl)cyclopropyl)isoxazol-5-yl)carbamate (Ic-E6, 0.087 g, 0.278 mmol), and was obtained as an off-white solid (0.011 g, 7% yield). ¹H NMR (400 MHz, DMSO-d₆) δ=10.53 (bs, 1H), 8.78 (bs, 1H), 8.05-8.09 (m, 2H), 7.32 (s, 1H), 7.19-7.28 (m, 2)), 6.18 (bs, 2H), 6.13 (s, 1H)), 4.90 (t, J=5.2 Hz, 1H), 4.14 (t, J=6.0 Hz, 2H), 3.66-3.69 (m, 2H), 1.31-1.41 (m, 4H). LCMS: 506.2 [M+H].

### Example IC-53

### 1-(4-(4-amino-7-cyclobutyl-7H-pyrrolo[2,3-D]pyrimidin-5-yl)-2-fluorophenyl)-3-(3-(1-(trifluoromethyl)cyclopropyl)isoxazol-5-yl)urea

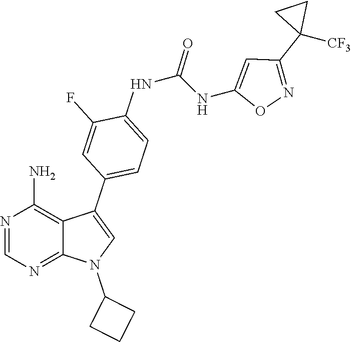 [SMILES: Nc1ncnc2c1c(-c1ccc(NC(=O)Nc3cc(C4(C(F)(F)F)CC4)no3)c(F)c1)cn2C1CCC1]

The title compound was obtained following the general procedure for urea formation (Method Ic-B), starting from 5-(4-amino-3-fluorophenyl)-7-cyclobutyl-7H-pyrrolo[2,3-d]pyrimidin-4-amine (Ic-D24, 0.150 g, 0.504 mmol) and phenyl (3-(1-(trifluoromethyl)cyclopropyl)isoxazol-5-yl)carbamate (Ic-E6, 0.158 g, 0.504 mmol), and was obtained as an off-white solid (0.111 g, 38% yield). ¹H NMR (400 MHz, DMSO-dl) δ=10.67 (bs, 1H), 8.96 (bs, 1H), 8.38 (s, 1H), 8.18-8.22 (m, 1H), 7.93 (s, 1H), 7.43 (bs, 2H), 7.41 (d, J=1.6 Hz, 1H), 7.32 (d, J=8.4 Hz, 1H), 6.21 (s, 1H), 5.23-5.27 (m, 1H), 2.51-2.68 (m, 4H), 1.84-1.89 (m, 2H), 1.38-1.49 (m, 4H). LCMS: 516.1 [M+H].

### Example IC-54

### 1-(4-(4-amino-7-cyclopropyl-7H-pyrrolo[2,3-d]pyrimidin-5-yl)-2-chlorophenyl)-3-(3-(1-(trifluoromethyl)cyclopropyl)isoxazol-5-yl)urea

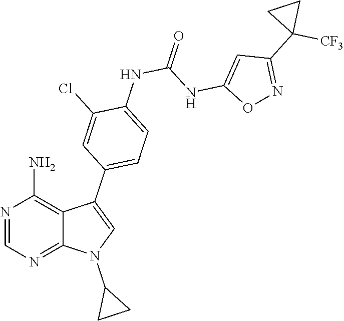 [SMILES: Nc1ncnc2c1c(-c1ccc(NC(=O)Nc3cc(C4(C(F)(F)F)CC4)no3)c(Cl)c1)cn2C1CC1]

The title compound was obtained following the general procedure for urea formation (Method Ic-B), starting from 5-(4-amino-3-chlorophenyl)-7-cyclopropyl-7H-pyrrolo[2,3-d]pyrimidin-4-amine (Ic-D25, 0.050 g, 0.167 mmol) and phenyl (3-(1-(trifluoromethyl)cyclopropyl)isoxazol-5-yl)carbamate (Ic-E6, 0.052 g, 0.167 mmol), and was obtained as an off-white solid (6 mg, 7% yield). ¹H NMR (400 MHz, DMSO-d)=11.04 (bs, 1H), 8.67 (bs, 1H), 8.40 (s, 1H), 8.23-8.25 (m, 1H), 7.58-7.60 (m, 2H), 7.42-7.44 (m, 1H), 6.21 (s, 1H), 3.67-3.73 (m, 11H), 1.46-2.33 (m, 2H), 1.37-1.38 (m, 2H), 1.11-1.13 (m, 4H). LCMS: 518.2 [M+H].

### Example IC-55

### 1-(4-(4-amino-7-cyclopropyl-7H-pyrrolo[2,3-d]pyrimidin-5-yl)-2-methoxyphenyl)-3-(3-(1-(trifluoromethyl)cyclopropyl)isoxazol-5-yl)urea

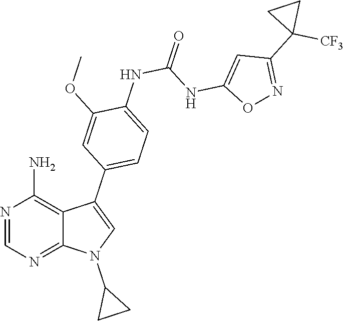 [SMILES: COc1cc(-c2cn(C3CC3)c3ncnc(N)c23)ccc1NC(=O)Nc1cc(C2(C(F)(F)F)CC2)no1]

The title compound was obtained following the general procedure for urea formation (Method Ic-B), starting from 5-(4-amino-3-methoxyphenyl)-7-cyclopropyl-7H-pyrrolo[2,3-d]pyrimidin-4-amine (Ic-D26, 0.020 g, 0.068 mmol) and phenyl (3-(1-(trifluoromethyl)cyclopropyl)isoxazol-5-yl)carbamate (Ic-E6, 0.021 g, 0.068 mmol), and was obtained as an off-white solid (7 mg, 19% yield). ¹H NMR (400 MHz, DMSO-d₆) δ=10.96 (bs, 1H), 8.65 (bs, 1H), 8.23 (s, 1H), 8.17 (d, J=8.4 Hz, 1H), 7.30 (s, 1H), 7.12-7.17 (m, 1H), 7.01-7.04 (m, 1H), 6.47 (bs, 2H), 6.19 (s, 1H), 3.94 (s, 3H), 3.60-3.62 (m, 1H), 1.45-1.48 (m, 2H), 1.36-1.38 (m, 2H), 1.04-1.07 (m, 4H). LCMS: 513.9 [M+H].

### Example IC-56

### 1-(4-(4-amino-7-(1-methylpyrrolidin-3-yl)-7H-pyrrolo[2,3-d]pyrimidin-5-yl)-2-fluorophenyl)-3-(3-(1-(trifluoromethyl)cyclopropyl)isoxazol-5-yl)urea

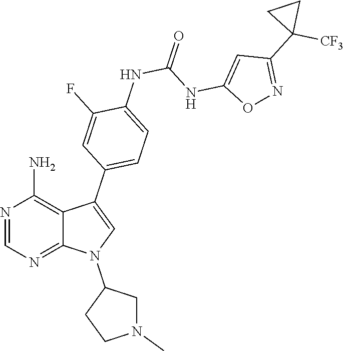 [SMILES: CN1CCC(n2cc(-c3ccc(NC(=O)Nc4cc(C5(C(F)(F)F)CC5)no4)c(F)c3)c3c(N)ncnc32)C1]

The title compound was obtained following the general procedure for urea formation (Method Ic-B), starting from 5-(4-amino-3-fluorophenyl)-7-(1-methylpyrrolidin-3-yl)-7H-pyrrolo[2,3-d]pyrimidin-4-amine (Ic-D27, 0.040 g, 0.123 mmol) and phenyl (3-(1-(trifluoromethyl)cyclopropyl)isoxazol-5-yl)carbamate (Ic-E6, 0.036 g, 0.115 mmol), and was obtained as a white solid (0.015 mg, 24% yield). ¹H NMR (400 MHz, DMSO-d₆) δ=10.82 (bs, 1H), 9.07 (bs, 1H), 8.39 (s, 1H), 8.19-8.23 (m, 1H), 7.84 (s, 1H), 7.14-7.40 (m, 2H), 6.21 (s, 1H), 5.55-5.66 (m, 1H), 3.90-4.09 (m, 2H), 2.95 (bs, 4H), 2.08 (s, 3H), 1.38-1.49 (m, 4H). LCMS: 545.3 [M+H].

### Example IC-57

### 1-(4-(4-amino-7-cyclopropyl-7H-pyrrolo[2,3-d]pyrimidin-5-yl)-2-fluorophenyl)-3-(5-(1-(trifluoromethyl)cyclobutyl)isoxazol-3-yl)urea

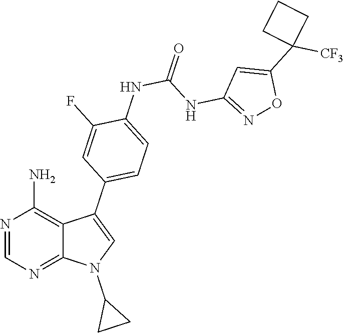 [SMILES: Nc1ncnc2c1c(-c1ccc(NC(=O)Nc3cc(C4(C(F)(F)F)CCC4)on3)c(F)c1)cn2C1CC1]

The title compound was obtained following the general procedure for urea formation (Method Ic-A), starting from 5-(4-amino-3-fluorophenyl)-7-cyclopropyl-7H-pyrrolo[2,3-d]pyrimidin-4-amine (Ic-D1, 0.160 g, 0.565 mmol) and phenyl (5-(1-(trifluoromethyl)cyclobutyl)isoxazol-3-yl)carbamate (Ic-E25, 0.184 g, 0.565 mmol), and was obtained as a white solid (0.081 mg, 28% yield). ¹H NMR (400 MHz, DMSO-d₆) δ=10.02 (bs, 1H), 8.87 (bs, 1H), 8.15-8.20 (m, 2H), 7.25-7.36 (m, 3H), 6.96 (s, 1H), 6.16 (bs, 2H), 3.55-3.61 (m, 1H), 2.59-2.68 (m, 4H)), 2.03-2.11 (m, 2H), 1.02-1.11 (m, 4H). LCMS: 516.2 [M+H].

### Example IC-58

### 1-(4-(4-amino-7-cyclobutyl-7H-pyrrolo[2,3-d]pyrimidin-5-yl)-2-fluorophenyl)-3-(5-(1-(trifluoromethyl)cyclopropyl)isoxazol-3-yl)urea

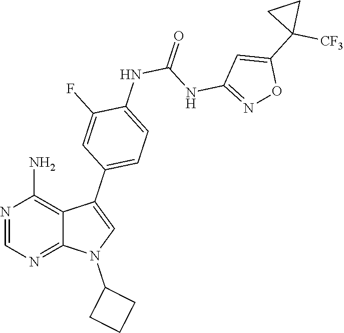 [SMILES: Nc1ncnc2c1c(-c1ccc(NC(=O)Nc3cc(C4(C(F)(F)F)CC4)on3)c(F)c1)cn2C1CCC1]

The title compound was obtained following the general procedure for urea formation (Method Ic-B), starting from 5-(4-amino-3-fluorophenyl)-7-cyclobutyl-7H-pyrrolo[2,3-d]pyrimidin-4-amine (Ic-D24, 0.204 g, 0.686 mmol) and phenyl (5-(1-(trifluoromethyl)cyclopropyl)isoxazol-3-yl)carbamate (Ic-E7, 0.214 g, 0.686 mmol), and was obtained as an off-white solid (0.096 g, 27% yield). ¹H NMR (400 MHz, DMSO-d₆) δ=10.01 (bs, 1H), 8.87 (bs, 1H), 8.14-8.20 (m, 2H), 7.66 (s, 1H), 7.28-7.39 (m, 2H), 6.91 (s, 1H), 6.18 (bs, 2H), 5.18-5.23 (m, 1H), 2.39-2.41 (m, 4H), 1.81-1.90 (m, 2H), 1.54-1.55 (m, 4H). LCMS: 516.2 [M+H].

### Example IC-59

### 1-(6-(4-amino-7-cyclopropyl-7H-pyrrolo[2,3-d]pyrimidin-5-yl)pyridin-3-yl)-3-(3-(1-(trifluoromethyl)cyclopropyl)isoxazol-5-yl)urea

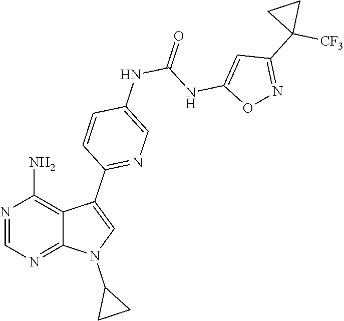 [SMILES: Nc1ncnc2c1c(-c1ccc(NC(=O)Nc3cc(C4(C(F)(F)F)CC4)no3)cn1)cn2C1CC1]

The title compound was obtained following the general procedure for urea formation (Method Ic-B), starting from 5-(5-aminopyridin-2-yl)-7-cyclopropyl-7H-pyrrolo[2,3-d]pyrimidin-4-amine (Ic-D14, 0.030 g, 0.045 mmol) and phenyl (3-(1-(trifluoromethyl)cyclopropyl)isoxazol-5-yl)carbamate (Ic-E6, 0.014 g, 0.045 mmol), and was obtained as an off-white solid (5 mg, 23% yield). ¹H NMR (400 MHz, DMSO-d₆) δ=10.67 (bs, 1H), 9.68 (bs, 1H), 9.12 (s, 1H), 8.59-8.60 (m, 1), 8.09 (s, 1H), 7.92-8.00 (m, 2H), 7.17 (bs, 2H), 6.20 (s, 1H), 3.58-3.63 (m, 1H), 1.36-1.48 (m, 4H), 1.06-1.10 (m, 4H). LCMS: 485.2 [M+H].

### Example IC-60

### 1-(4-(4-amino-7-cyclopropyl-7H-pyrrolo[2,3-d]pyrimidin-5-yl)-2,6-difluorophenyl)-3-(5-(1-(trifluoromethyl)cyclopropyl)isoxazol-3-yl)urea

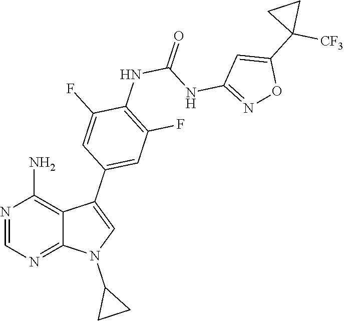 [SMILES: Nc1ncnc2c1c(-c1cc(F)c(NC(=O)Nc3cc(C4(C(F)(F)F)CC4)on3)c(F)c1)cn2C1CC1]

The title compound was obtained following the general procedure for urea formation (Method Ic-A), starting from 5-(4-amino-3,5-difluorophenyl)-7-cyclopropyl-71-pyrrolo[2,3-d]pyrimidin-4-amine (Ic-D15, 0.150 g 0.498 mmol) and phenyl (5-(1-(trifluoromethyl)cyclopropyl)isoxazol-3-yl)carbamate (Ic-E7, 0.155 g, 0.498 mmol), and was obtained as a white solid (0.015 g, 6% yield). ¹H NMR (400 MHz, DMSO-d₆) δ=11.47 (bs, 1H), 10.17 (bs, 1H), 8.18 (s, 1H), 7.42 (s, 1H), 7.18-7.22 (m, 2H), 6.82 (s, 1H), 6.27 (bs, 2H), 3.51-3.55 (m, 1H), 1.46-1.51 (m, 4H), 1.04-1.05 (m, 4H). LCMS: 520.2 [M+H].

### Example IC-61

### 1-(4-(4-amino-7-(2-hydroxyethyl)-7H-pyrrolo[2,3-d]pyrimidin-5-yl)-2-fluorophenyl)-3-(5-(1-(trifluoromethyl)cyclopropyl)isoxazol-3-yl)urea

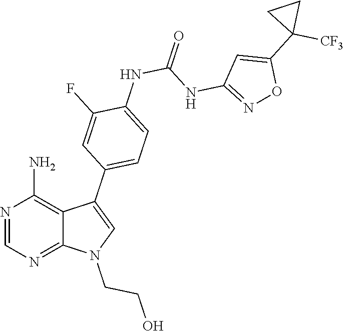 [SMILES: Nc1ncnc2c1c(-c1ccc(NC(=O)Nc3cc(C4(C(F)(F)F)CC4)on3)c(F)c1)cn2CCO]

The title compound was obtained following the general procedure for urea formation (Method Ic-B), starting from 2-(4-amino-5-(4-amino-3-fluorophenyl)-7H-pyrrolo[2,3-d]pyrimidin-7-yl)ethan-1-ol (Ic-D23, 0.100 g, 0.348 mmol) and phenyl (5-(1-(trifluoromethyl)cyclopropyl)isoxazol-3-yl)carbamate (Ic-E7, 0.109 g, 0.348 mmol), and was obtained as a white solid (0.035 mg, 20% yield). ¹H NMR (400 MHz, CD₃OD) δ=8.35 (s, 1H), 8.23-8.27 (m, 1H), 7.58 (s, 1H), 7.32-7.39 (m, 2H), 6.81 (s, 1H), 4.47 (t, J=10.8 Hz, 2H), 3.95-3.98 (m, 2H), 1.49-1.59 (m, 4H). LCMS: 506.2 [M+H].

### Example IC-62

### 1-(4-(4-amino-1-cyclopropyl-1H-pyrrolo[3,2-c]pyridin-3-yl)-2-fluorophenyl)-3-(3-(1-(trifluoromethyl)cyclopropyl)isoxazol-5-yl)urea

### Step 1: Synthesis of 1-cyclopropyl-N-(2,4-dimethoxybenzyl)-3-iodo-1H-pyrrolo[3,2-c]pyridin-4-amine

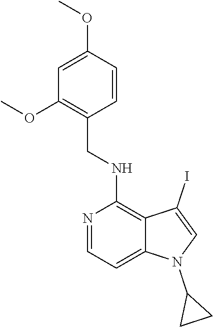 [SMILES: COc1ccc(CNc2nccc3c2c(I)cn3C2CC2)c(OC)c1]

A mixture of 4-chloro-1-cyclopropyl-3-iodo-1H-pyrrolo[3,2-c]pyridine (Ic-B9, 0.300 g, 0.942 mmol) and (2,5-dimethoxyphenyl)methanamine (0.429 mL, 2.83 mmol) in n-BuOH (10 mL) was stirred at 110° C. for 12 h. Following completion of the reaction (as indicated by LCMS), the reaction mixture was concentrated under reduced pressure to give crude material which was purified by Isolera (silica gel 230-400 mesh, eluting with 30% EtOAc in petroleum ether). Affording the title product as a yellow gum (0.10 g, 19% yield). LCMS: 450.0 [M+H].

### Step 2: Synthesis of 3-(4-amino-3-fluorophenyl)-1-cyclopropyl-N-(2,4-dimethoxybenzyl)-1H-pyrrolo[3,2-c]pyridin-4-amine

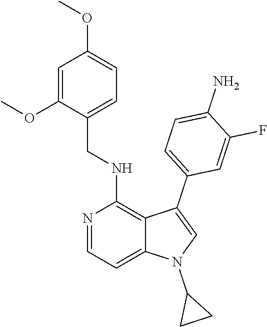 [SMILES: COc1ccc(CNc2nccc3c2c(-c2ccc(N)c(F)c2)cn3C2CC2)c(OC)c1]

The title compound was obtained by following a similar procedure described for Intermediate Ic-D8, starting from 1-cyclopropyl-N-(2,4-dimethoxybenzyl)-3-iodo-1H-pyrrolo[3,2-c]pyridin-4-amine (0.190 g, 0.423 mmol) and 2-fluoro-4-(4,4,5,5-tetramethyl-1,3,2-dioxaborolan-2-yl)aniline (0.100 g, 0.423 mmol), and was obtained as a brown gum (0.080 g, 41% yield). LCMS: 433.2 [M+H].

### Step 3: Synthesis of 1-(4-(1-cyclopropyl-4-((2,4-dimethoxybenzyl)amino)-1H-pyrrolo[3,2-c]pyridin-3-yl)-2-fluorophenyl)-3-(3-(1-(trifluoromethyl)cyclopropyl)isoxazol-5-yl)urea

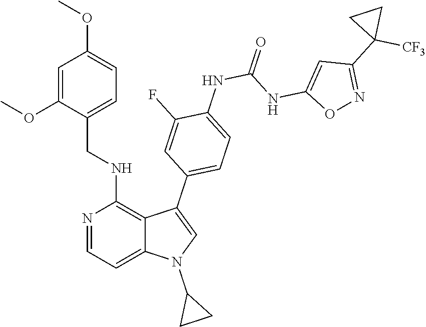 [SMILES: COc1ccc(CNc2nccc3c2c(-c2ccc(NC(=O)Nc4cc(C5(C(F)(F)F)CC5)no4)c(F)c2)cn3C2CC2)c(OC)c1]

The title compound was obtained following the general procedure for urea formation (Method Ic-B), starting from 3-(4-amino-3-fluorophenyl)-1-cyclopropyl-N-(2,4-dimethoxybenzyl)-Ili-pyrrolo[3,2-c]pyridin-4-amine (0.080 g, 0.185 mmol) and phenyl (3-(1-(trifluoromethyl)cyclopropyl)isoxazol-5-yl)carbamate (Ic-E6, 0.058 g, 0.185 mmol), and was obtained as an off-white solid (0.027 g, 17% yield). LCMS: 651.3 [M+H].

### Step 4: Synthesis of 1-(4-(4-amino-1-cyclopropyl-1H-pyrrolo[3,2-c]pyridin-3-yl)-2-fluorophenyl)-3-(3-(1-(trifluoromethyl)cyclopropyl)isoxazol-5-yl)urea

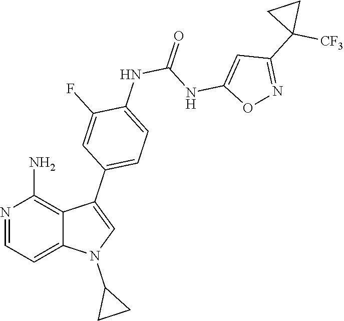 [SMILES: Nc1nccc2c1c(-c1ccc(NC(=O)Nc3cc(C4(C(F)(F)F)CC4)no3)c(F)c1)cn2C1CC1]

Triethylsilane (4.8 mg, 0.041 mmol) and TFA (4.7 mg, 0.041 mmol) were added to a solution of 1-(4-(1-cyclopropyl-4-((2,4-dimethoxybenzyl)amino)-1H-pyrrolo[3,2-c]pyridin-3-yl)-2-fluorophenyl)-3-(3-(1-(trifluoromethyl)cyclopropyl)isoxazol-5-yl)urea (0.027 g, 0.041 mmol) in DCM (2 mL) at 0° C. and the resulting mixture was stirred at 25° C. for 12 h. Following completion of the reaction (as indicated by LCMS), the reaction mixture was concentrated under reduced pressure to give crude material which was purified by preparative HPLC (eluting with a gradient of 0.1% TFA in water and), affording the title product as an off-white solid (5 mg, 24% yield). ¹H NMR (400 MHz, CD₃OD) δ=8.21-8.25 (m, 1H), 7.64-7.66 (m, 1H), 7.51 (s, 1H), 7.30-7.38 (m, 3H), 6.33 (s, 1H), 3.59-3.62 (m, 1H), 1.39-1.49 (m, 4H), 1.13-1.26 (m, 4H), LCMS: 501.2 [M+H].

### Intermediate ID-A1

### 1-cyclopropyl-3-iodo-1H-pyrazolo[3,4-d]pyrimidin-4-amine

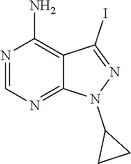 [SMILES: Nc1ncnc2c1c(I)nn2C1CC1]

Copper (II) acetate (0.348 g, 1.916 mmol), 2,2′-bipyridine (0.299 g, 1.916 mmol), and sodium bicarbonate (0.322 g, 3.830 mmol) were added to a stirred solution of 3-iodo-1H-pyrazolo[3,4-d]pyrimidin-4-amine (0.500 g, 1.916 mmol) and cyclopropylboronic acid (0.329 g, 3.830 mmol) in dichloroethane (10 mL). The resulting mixture was stirred at 70° C. under oxygen atmosphere for 12 h. Following completion of the reaction (as indicated by TLC), the reaction mixture was filtered through a pad of diatomaceous earth which was then rinsed with DCM (20 mL×2). The combined filtrates were washed with water (20 mL) and brine (25 mL), the organic layer separated, dried over Na₂SO₄, filtered, and concentrated under reduced pressure to yield crude material which was purified by flash chromatography (silica gel 230-400 mesh, eluting with 20% EtOAc in petroleum ether), giving the title compound as an off-white solid (0.24 g, 36% yield). ¹H NMR (400 MHz, DMSO-d₆) δ=8.21 (s, 1H), 3.74-3.79 (m, 1H), 1.11-1.15 (m, 2H), 1.04-1.09 (m, 2H); LCMS: 301.8 [M+H].

### Intermediate ID-A2

### 3-iodo-1-isopropyl-1H-pyrazolo[3,4-d]pyrimidin-4-amine

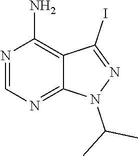

Cs₂CO₃ (12.38 g, 38.31 mmol) and 2-iodopropane (3.60 g, 21.16 mmol) were added to a stirred solution of 3-iodo-1H-pyrazolo[3,4-d]pyrimidin-4-amine (5.00 g, 19.15 mmol) in DMF (25 mL) in a sealed 25 mL tube. The reaction mixture was stirred at 90° C. for 16 h and, following completion of the reaction (as indicated by TLC), was poured into crushed ice (50 g) and stirred for 15 min. The resulting solid was filtered, washed with water (2×5 mL), and dried to afford the title compound as an off-white solid (3.25 g, 56% yield). ¹H NMR (400 MHz, DMSO-d₆) δ=8.18 (s, 1H), 4.93-4.99 (m, 1H), 1.42 (d, J=6.8 Hz, 6H); LCMS: 303.8 [M+H].

### Intermediate ID-A3

### 3-iodo-1-(oxetan-3-yl)-1H-pyrazolo[3,4-d]pyrimidin-4-amine

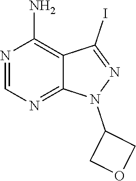 [SMILES: Nc1ncnc2c1c(I)nn2C1COC1]

Intermediate Id-A3 was prepared via a similar procedure described for Intermediate Id-A2, replacing 2-iodopropane with 3-iodooxetane. ¹H NMR (400 MHz, DMSO-d₆) δ=8.21 (s, 1H), 5.89-5.93 (m, 1H), 4.93-5.00 (m, 4H). LCMS: 318.0 [M+H].

### Intermediate ID-A4

### 1-(4-amino-3-iodo-1H-pyrazolo[3,4-d]pyrimidin-1-yl)-2-methylpropan-2-ol

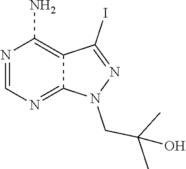

NaH₂PO₄ (0.044 g, 0.372 mmol) was added to a mixture of 3-iodo-1H-pyrazolo[3,4-d]pyrimidin-4-amine (0.100 g, 0.380 mmol), 2,2-dimethyloxirane (0.055 g, 0.760 mmol), and K₂CO₃ (0.050 g, 0.372 mmol) in acetonitrile (3 mL) and water (1 mL) and the resulting solution subjected to microwave irradiation at 150° C. for 1 h. After completion of the reaction (as indicated by TLC), the solvents were removed under reduced pressure to yield crude material which was purified by flash chromatography (silica gel 230-400 mesh, eluting with 25% EtOAc in petroleum ether), giving the title compound as a pale brown solid (0.064 g, 51% yield). ¹H NMR (400 MHz, DMSO-d₆) δ=8.20 (s, 1H), 4.19 (s, 2H), 1.09 (s, 6H). LCMS: 334.0 [M+H].

### Intermediate ID-A5

### 7-cyclopropyl-5-iodo-7H-pyrrolo[2,3-d]pyrimidin-4-amine

### Step 1: Synthesis of 4-chloro-7-cyclopropyl-7H-pyrrolo[2,3-d]pyrimidine

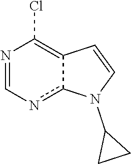

Sodium bicarbonate (10.94 g, 130.0 mmol), 2,2′-bipyridine (10.17 g, 65.1 mmol), and copper (II) acetate (11.83 g, 65.1 mmol) were added to a solution of 4-chloro-7H-pyrrolo[2,3-d]pyrimidine (10.00 g, 65.1 mmol) and cyclopropylboronic acid (11.19 g, 130.0 mmol) in dichloroethane (150 mL) and the resulting mixture was stirred at 80° C. Following completion of the reaction (as indicated by TLC), the reaction mixture was filtered through a pad of diatomaceous earth which was then rinsed with DCM (2×100 mL). The combined filtrates were washed with water (100 mL) and brine (100 mL), dried over Na₂SO₄, filtered, and concentrated under reduced pressure to yield crude material which was purified by GRACE (silica gel 230-400 mesh, eluting with 6% EtOAc in petroleum ether), giving the title compound as an off-white solid (7.30 g, 58% yield). ¹H NMR (400 MHz, DMSO-d₆) δ=8.65 (s, 1H), 7.70 (d, J=3.6 Hz, 1H), 6.59 (d, J=4.0 Hz, 1H), 3.62-3.68 (m, 1H), 1.08-1.10 (m, 4H). LCMS: 194.0 [M+H].

### Step 2: Synthesis of 4-chloro-7-cyclopropyl-5-iodo-7H-pyrrolo[2,3-d]pyrimidine

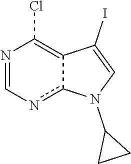

N-iodosuccinimide (8.25 g, 36.7 mmol) was added to a solution of 4-chloro-7-cyclopropyl-7H-pyrrolo[2,3-d]pyrimidine (7.10 g, 36.7 mmol) in DMF (50 mL) at 0° C. and the resulting mixture was stirred at 25° C. for 12 h. Following completion of the reaction (as indicated by TLC), the reaction mixture was poured into ice cold water (600 mL) and stirred at 25° C. for 15 min. The resulting precipitate was filtered, washed with water (2×250 mL), and dried under reduced pressure to afford the title product (8.30 g, 70% yield) as a pale brown solid. ¹H NMR (400 MHz, DMSO-d₆) δ=8.65 (s, 1H), 7.94 (s, 1H), 3.62-3.68 (m, 1H), 1.05-1.10 (m, 1H). LCMS: 319.9 [M+H].

### Step 3: Synthesis of 7-cyclopropyl-5-iodo-7H-pyrrolo[2,3-d]pyrimidin-4-anine

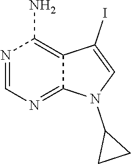

A mixture of 4-chloro-7-cyclopropyl-5-iodo-7H-pyrrolo[2,3-d]pyrimidine (8.3 g, 26 mmol) and ammonium hydroxide (25% in water, 25 mL) in 1,4-dioxane (25 mL) was loaded in a tinyclave and stirred at 120° C. for 16 h. Following completion of the reaction (as indicated by TLC), the reaction mixture was cooled to 25° C. and the resulting precipitate was filtered, washed with petroleum ether (2×50 mL), and dried to afford the title compound as an off-white solid (6.0 g, 76% yield). ¹H NMR (400 MHz, DMSO-d₆) δ=0.11 (s, 1H), 7.38 (s, 1H), 6.59 (bs, 2H), 3.48-3.54 (m, 1H), 0.97-0.99 (m, 4H). LCMS: 300.9 [M+H].

### Intermediate ID-A6

### 5-iodo-7-methyl-7H-pyrrolo[2,3-b]pyrimidin-4-amine

### Step 1: Synthesis of 4-chloro-5-iodo-7H-pyrrolo[2,3-d]pyrimidine

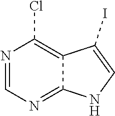

N-iodosuccinimide (7.33 g, 32.6 mmol) was added to a solution of 4-chloro-7H-pyrrolo[2,3-d]pyrimidine (5.00 g, 32.6 mmol) in DMF (20 mL) at 0° C. and the resulting mixture was stirred at 25° C. for 12 h. Following completion of the reaction (as indicated by TLC), the reaction mixture was poured into crushed ice (200 g) and stirred at 25° C. for 30 minutes. The resulting precipitate was filtered, washed with water (2.20 mL), and dried to afford the title compound as pale brown solid (8.52 g, 92% yield). ¹H NMR (400 MHz, DMSO-d₆) δ=12.96 (bs, 1H), 8.60 (s, 1H), 7.94 (s, 1H). LCMS: 280.0 [M+H].

### Step 2: Synthesis of 4-chloro-5-iodo-7-methyl-7H-pyrrolo[2,3-d]pyrimidine

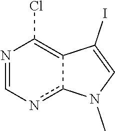

A solution of 4-chloro-5-iodo-7H-pyrrolo[2,3-d]pyrimidine (0.500 g, 1.789 mmol) in TH (5 mL) was added dropwise to a stirred suspension of NaH (60% in mineral oil, 0.143 g, 3.580 mmol) in THE (15 mL) at 0° C. and the resulting mixture was stirred at 0° C. for 30 min. Iodomethane (0.134 mL, 2.147 mmol) was then added and the resulting mixture was stirred at 25° C. for 4 h. Following completion of the reaction (as indicated by TLC), the reaction mixture was quenched with water (10 mL) and extracted with EtOAc (2×10 mL). The combined organic extracts were dried over Na₂SO₄, filtered, and concentrated under reduced pressure to afford the title compound as an off-white solid (0.38 g, 71% yield). LCMS: 294.0 [M+H].

### Step 3: Synthesis of 5-iodo-7-methyl-7H-pyrrolo[2,3-d]pyrimidin-4-amine

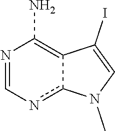

A mixture of 4-chloro-5-iodo-7-methyl-7H-pyrrolo[2,3-d]pyrimidine (0.40 g, 1.363 mmol) and aqueous ammonia (25% in water, 4 mL) in dioxane (4 mL) was subjected to microwave irradiation at 150° C. for 1 h. Following completion of the reaction (as indicated by LCMS), the reaction mixture was concentrated under reduced pressure to afford crude material which was purified by flash chromatography (silica gel 230-400 mesh, eluting with 10% MeOH in DCM), affording the title compound as an off-white solid (0.12 g, 32% yield). ¹H NMR (400 MHz, DMSO-d₆) δ=8.10 (s, 1H), 7.42 (s, 1H), 6.59 (bs, 2H), 3.67 (s, 3H). LCMS: 275.0 [M+H].

### Intermediate ID-A7

### 7-cyclobutyl-5-iodo-7H-pyrrolo[2,3-d]pyrimidin-4-amine

### Step 1: Synthesis of 4-chloro-5-iodo-7H-pyrrolo[2,3-d]pyrimidine

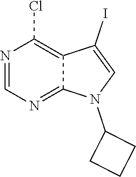

Bromocyclobutane (0.580 g, 4.29 mmol) and K₂CO₃ (0.742 g, 5.37 mmol) were added to a solution of 4-chloro-5-iodo-7H-pyrrolo[2,3-d]pyrimidine (1.000 g, 3.58 mmol) in DMF (5 mL) and the resulting mixture was stirred at 80° C. for 16 h. Following completion of the reaction (as indicated by TLC), the reaction mixture was poured into crushed ice (25 g). The resulting precipitate was filtered, washed with water (20 mL), and dried to give crude material which was purified by Isolera (silica gel 230-400 mesh, eluting with 20% EtOAc in petroleum), affording the title compound as an off-white solid (0.35 g, 29% yield). LCMS: 333.9 [M+H].

### Step 2: Synthesis of 7-cyclobutyl-5-iodo-7H-pyrrolo[2,3-d]pyrimidin-4-amine

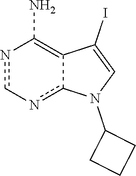

A mixture of 4-chloro-7-cyclobutyl-5-iodo-7H-pyrrolo[2,3-d]pyrimidine (0.35 g, 1.049 mmol) and aqueous ammonia (25% in water, 4.5 mL) in dioxane (4.5 mL) was subjected to microwave irradiation at 150° C. for 1 h. Following completion of the reaction (as indicated by TLC), the reaction mixture was concentrated under reduced pressure to afford the title compound as an off-white solid (0.30 g, 83% yield) which was used without further purification. ¹H NMR (400 MHz, DMSO-d₆) δ=8.09 (s, 1H), 7.73 (s, 1H), 6.61 (bs, 2H), 5.09-5.16 (m, 1H), 2.50-2.50 (m, 2H), 2.31-2.38 (m, 2H), 1.75-1.83 (m, 2H) LCMS: 315.0 [M+H].

### Intermediate ID-B1

### 3-(4-amino-3-fluorophenyl)-1-cyclopropyl-1H-pyrrolo[3,4-d]pyrimidin-4-amine

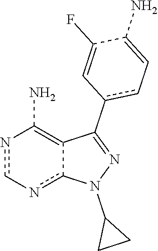

A mixture of 1-cyclopropyl-3-iodo-1H-pyrazolo[3,4-d]pyrimidin-4-amine (Id-A1, 0.500 g, 1.66 mmol), 2-fluoro-4-(4,4,5,5-tetramethyl-1,3,2-dioxaborolan-2-yl)aniline (0.433 g, 1.82 mmol), and K₂CO₃ (0.688 g, 4.98 mmol) in 1,4-dioxane (25 mL) and water (2.5 mL) was purged with N₂ for 10 min. Pd(PPh₃)₄ (0.092 g, 0.08 mmol) was then added and the resulting mixture was stirred at 100° C. for 16 h. Following completion of the reaction (as indicated by TLC), the reaction mixture was filtered through a pad of diatomaceous earth which was then rinsed with EtOAc (2.10 mL). The combined filtrate were concentrated under reduced pressure to yield crude material which was purified by flash chromatography (silica gel 230-400 mesh, eluting with 2% MeOH in DCM), affording the title compound as a yellow solid (0.46 g, 98% yield). ¹H NMR (400 MHz, DMSO-d₆) δ=8.23 (s, 1H), 7.15-7.24 (m, 2H), 6.87-6.91 (m, 1H), 5.47 (bs, 2H), 3.80-3.84 (m, 1H), 1.18-1.19 (m, 2H), 1.05-1.08 (m, 2H), LCMS: 285.0 [M+H].

### Intermediate ID-B2

### 3-(4-aminophenyl)-1-isopropyl-1H-pyrazolo[3,4-d]pyrimidin-4-amine

### Step 1: Synthesis of 1-isopropyl-3-(4-nitrophenyl)-1H-pyrazolo[3,4-d]pyrimidin-4-amine

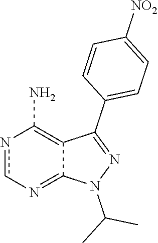

The title compound was prepared via a similar procedure described for Intermediate Id-B1, starting from 3-iodo-1-isopropyl-1H-pyrazolo[3,4-d]pyrimidin-4-amine (Id-A2, 1.887 g, 6.23 mmol) and (4-nitrophenyl)boronic acid (1.560 g, 9.34 mmol), and was obtained as a yellow solid (1.242 g, 67% yield). ¹H NMR (400 MHz, DMSO-d₆) δ=8.38-8.40 (m, 2H), 8.28 (s, 1H), 7.92-7.95 (m, 2H), 5.07-5.14 (m, 1H), 1.51 (d, J=6.8 Hz, 6H). LCMS: 299.1 [M+H].

### Step 2: Synthesis of 3-(4-aminophenyl)-1-isopropyl-11-pyrazolo[3,4-d]pyrimidin-4-amine

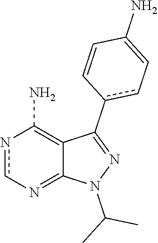

Iron powder (2.320 g, 41.60 mmol) and ammonium chloride (2.220 g, 41.60 mmol) were added to a stirred solution of 1-isopropyl-3-(4-nitrophenyl)-1H-pyrazolo[3,4-d]pyrimidin-4-amine (1.242 g, 4.16 mmol) in ethanol (50 mL) and water (20 mL) and the resulting mixture was heated to 80° C. for 3 h. After completion of the reaction (as indicated by TLC), the reaction mixture was filtered through a pad of diatomaceous earth which was then rinsed with EtOAc (2×25 mL). The combined filtrates were concentrated under reduced pressure, the residue was dissolved in EtOAc (100 mL), washed with brine (25 mL), dried over Na₂SO₄, filtered, and evaporated under reduced pressure to give the title compound as a pale yellow solid (1.042 g, quantitative yield) which was taken forward without further purification.

### Intermediate ID-13

### 3-(4-aminophenyl)-1-(oxetan-3-yl)-1H-pyrazolo[3,4-d]pyrimidin-4-amine

### Step 1: Synthesis of 3-(4-nitrophenyl)-1-(oxetan-3-yl)-1H-pyrazolo[3,4-d]pyrimidin-4-amine

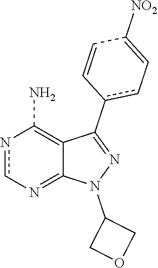

The title compound was prepared via a similar procedure described for Intermediate Id-B1, starting from 3-iodo-1-(oxetan-3-yl)-1H-pyrazolo[3,4-d]pyrimidin-4-amine (Id-A3, 0.800 g, 2.522 mmol) and (4-nitrophenyl)boronic acid (0.632 g, 3.78 mmol), and was obtained as a yellow solid (0.596 g, 76% yield). ¹H NMR (400 MHz, DMSO-d₆) δ=8.41-8.43 (m, 2H), 8.30 (s, 1H), 7.99-8.01 (m, 2H), 6.05-6.08 (m, 1H), 4.97-5.12 (m, 4H). LCMS: 31.1.0 [M−H].

### Step 2: Synthesis of 3-(4-aminophenyl)-1-(oxetan-3-yl)-1H-pyrazolo[3,4-d]pyrimidin-4-amine

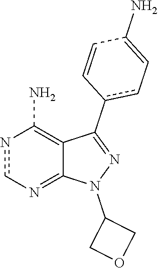

The title compound was prepared via a similar procedure described for step 2 of Intermediate Id-B2, starting from 3-(4-nitrophenyl)-1-(oxetan-3-yl)-1H-pyrazolo[3,4-d]pyrimidin-4-amine (0.596 g, 1.91 mmol) and Fe/NH₄Cl, and was obtained as a pale yellow solid (0.42 g, quantitative yield) which was taken forward without further purification. LCMS: 283.0 [M+H].

### Intermediate ID-B4

### 1-(4-amino-3-(4-amino-3-fluorophenyl)-1H-pyrazolo[3,4-d]pyrimidin-1-yl)-2-methylpropan-2-ol

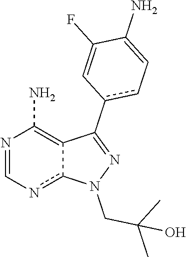

The title compound was prepared via a similar procedure described for Intermediate Id-B1, starting from 1-(4-amino-3-iodo-1H-pyrazolo[3,4-d]pyrimidin-1-yl)-2-methylpropan-2-ol (Id-A4, 0.110 g, 0.330 mmol) and 2-fluoro-4-(4,4,5,5-tetramethyl-1,3,2-dioxaborolan-2-yl)aniline (0.094 g, 0.396 mmol), and was obtained as a pale yellow solid (0.077 g, 66% yield). LCMS: 317.1 [M−H].

### Intermediate ID-B5

### 5-(4-amino-3-fluorophenyl)-7-cyclopropyl-7H-pyrrolo[2,3-d]pyrimidin-4-amine

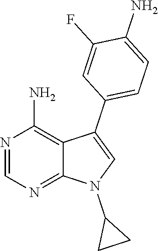 [SMILES: Nc1ccc(-c2cn(C3CC3)c3ncnc(N)c23)cc1F]

A mixture of 7-cyclopropyl-5-iodo-7H-pyrrolo[2,3-d]pyrimidin-4-amine (Id-A5, 10.5 g, 35.0 mmol), 2-fluoro-4-(4,4,5,5-tetramethyl-1,3,2-dioxaborolan-2-yl)aniline (9.12 g, 38.5 mmol), and K₂CO₃ (9.67 g, 70.0 mmol) in 1,4-dioxane (130 mL), EtOH (60 mL), and water (74.2 mL) was purged with N₂ for 10 min. PdCl₂(dppf) (1.280 g, 1.0.749 mmol) was then added and the resulting mixture was stirred at 80° C. for 3 h. Following completion of the reaction (as indicated by TLC), the reaction mixture was filtered through a pad elite which was then washed with EtOAc (2.100 mL). The combined filtrates were concentrated under reduced pressure to give crude material which was purified by GRACE (silica gel 230-400 mesh, eluting with 3% MeOH in DCM), affording the title compound as a yellow solid (5.7 g, 57% yield). ¹H NMR (400 MHz, DMSO-d₆) δ=8.13 (s, 1H), 7.12 (s, 1H), 7.05-7.09 (m, 1H), 6.95-6.98 (m, 1H), 6.82-6.87 (m, 1H), 6.04 (bs, 2H), 5.22 (bs, 2H), 3.53-3.56 (m, 1H), 1.02-1.04 (m, 4H). LCMS: 284.1 [M+H].

### Intermediate ID-B6

### 5-(4-amino-3-fluorophenyl)-7-methyl-7H-pyrrolo[2,3-d]pyrimidin-4-amine

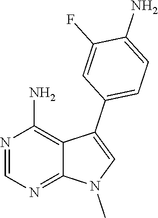 [SMILES: Cn1cc(-c2ccc(N)c(F)c2)c2c(N)ncnc21]

The title compound was prepared via a similar procedure described for Intermediate Id-B5, starting from 5-iodo-7-methyl-7H-pyrrolo[2,3-d]pyrimidin-4-amine (Id-A6, 0.120 g, 0.438 mmol) and 2-fluoro-4-(4,4,5,5-tetramethyl-1,3,2-dioxaborolan-2-yl)aniline (0.114 g, 0.482 mmol), and was obtained as a pale yellow solid (0.09 g, 65% yield). LCMS: 258.1 [M−H].

### Intermediate ID-B7

### 5-(4-amino-3-fluorophenyl)-7-cyclobutyl-7H-pyrrolo[2,3-d]pyrimidin-4-amine

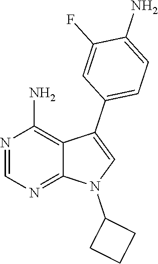 [SMILES: Nc1ccc(-c2cn(C3CCC3)c3ncnc(N)c23)cc1F]

The title compound was prepared via a similar procedure described for Intermediate Id-B5, starting from 7-cyclobutyl-5-iodo-7H-pyrrolo[2,3-d]pyrimidin-4-amine (Id-A7, 0.410 g, 1.305 mmol) and 2-fluoro-4-(4,4,5,5-tetramethyl-1,3,2-dioxaborolan-2-yl)aniline (0.309 g, 1.305 mmol), and was obtained as a brown solid (0.18 g, 37% yield). ¹H NMR (400 MHz, DMSO-d₆) δ=8.13 (s, 1H), 7.51 (s, 1H), 7.09-7.13 (m, 1H), 6.98-7.01 (m, 1H), 6.84-6.88 (m, 1H), 6.21 (bs, 2H), 5.14-5.26 (m, 3H), 2.50-2.51 (m, 2H), 2.34-2.39 (m, 2H), 1.85-1.92 (m, 2H). LCMS: 298.0 [M+H].

### Carbamate Intermediates Id-C

### General Procedure for the Synthesis of Carbamate Intermediates Id-C

Pyridine (1.2 eq) and phenyl chloroformate (1.5 eq) were added to a solution of amine (1.0 eq) in THF (10 vol) at 0° C. The reaction mixture was allowed to warm to 25° C. and was stirred for 16 h. Following completion of the reaction (as indicated by TLC):

(i) the precipitated solid was filtered, washed with THF, and dried to afford the desired carbamate OR

(ii) the reaction mixture was diluted with EtOAc (10 mL) and washed with brine (5 ml). The organic layer was dried over Na₂SO₄, filtered, and concentrated under reduced pressure to yield crude material which was purified by flash chromatography (silica gel 230-400 mesh, eluting with 10 to 20% EtOAc in petroleum ether), giving the desired carbamate.

The following carbamates were prepared using the above general procedure:

| | Carbamate structure | Amine structure | |
|---|---|---|---|
| Intermediate | (product) | (starting material) | LCMS |
| Id-C1 | 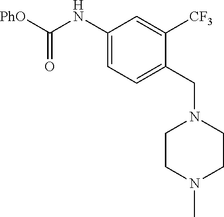 [SMILES: CN1CCN(Cc2ccc(NC(=O)Oc3ccccc3)cc2C(F)(F)F)CC1] | 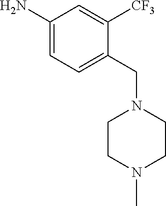 | 394.2 [M + H] |
| Id-C2 | 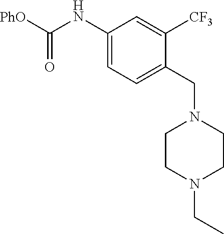 [SMILES: CCN1CCN(Cc2ccc(NC(=O)Oc3ccccc3)cc2C(F)(F)F)CC1] | 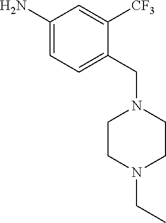 | 408.2 [M + H] |
| Id-C3 | 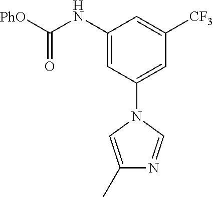 [SMILES: Cc1cn(-c2cc(NC(=O)Oc3ccccc3)cc(C(F)(F)F)c2)cn1] | 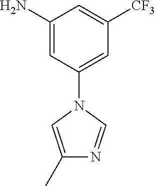 | 362.00 [M + H] |
| Id-C4 | 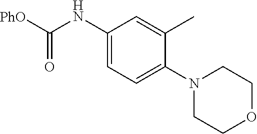 [SMILES: Cc1cc(NC(=O)Oc2ccccc2)ccc1N1CCOCC1] | 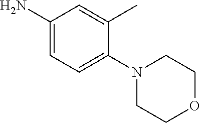 | 313.2 [M + H] |
| Id-C5 | 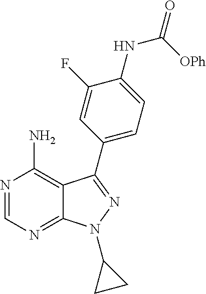 [SMILES: Nc1ncnc2c1c(-c1ccc(NC(=O)Oc3ccccc3)c(F)c1)nn2C1CC1] | 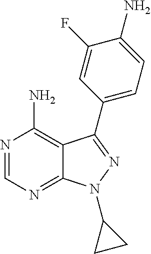 [SMILES: Nc1ccc(-c2nn(C3CC3)c3ncnc(N)c23)cc1F] | 405.1 [M + H] |
| Id-C6 | 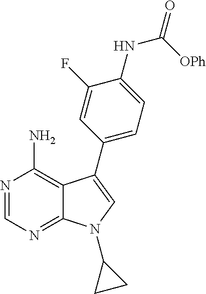 [SMILES: Nc1ncnc2c1c(-c1ccc(NC(=O)Oc3ccccc3)c(F)c1)cn2C1CC1] | 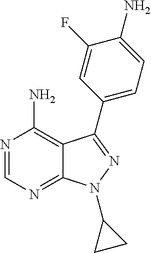 [SMILES: Nc1ccc(-c2nn(C3CC3)c3ncnc(N)c23)cc1F] | 404.2 [M + H] |
| Id-C7 | 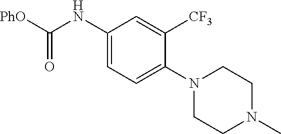 [SMILES: CN1CCN(c2ccc(NC(=O)Oc3ccccc3)cc2C(F)(F)F)CC1] | 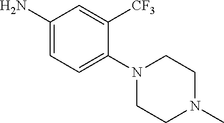 | 380.2 [M + H] |
| Id-C8 | 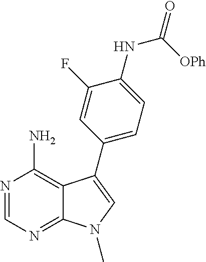 [SMILES: Cn1cc(-c2ccc(NC(=O)Oc3ccccc3)c(F)c2)c2c(N)ncnc21] | 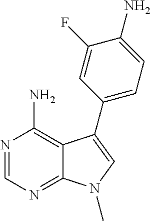 | 378.2 [M + H] |
| Id-C9 | 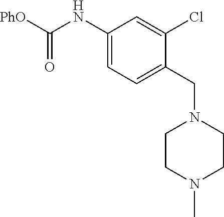 [SMILES: CN1CCN(Cc2ccc(NC(=O)Oc3ccccc3)cc2Cl)CC1] | 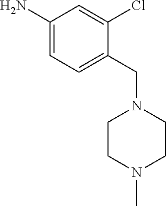 | 360.1 [M + H] |

All amines used for the synthesis of carbamate Intermediates Id-C are commercially available except for the following:

3-Methyl-4-morpholinoaniline (precursor to Id-C₄) was synthesized from 4-(2-methyl-4-nitrophenyl)morpholine as reported in J. Med. Chem. 2017, 60(12), 5099-5119.

3-Chloro-4-((4-methylpiperazin-1-yl)methyl)aniline (precursor to Id-C9) was synthesized from (4-amino-2-chlorophenyl)(4-methylpiperazin-1-yl)methanone as reported in PCT publication No. WO 2020/135507 A1.

### Amine Intermediates ID-D

| | | | |
|---|---|---|---|
| Intermediate | Id-D1 | Id-D2 | Id-D3 |
| Structure | 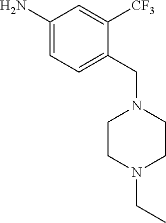 [SMILES: CCN1CCN(Cc2ccc(N)cc2C(F)(F)F)CC1] | 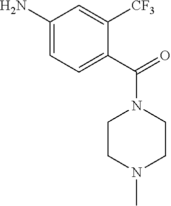 [SMILES: CN1CCN(C(=O)c2ccc(N)cc2C(F)(F)F)CC1] | 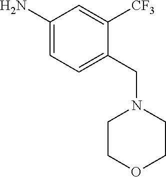 |
| Intermediate | Id-D4 | Id-D5 | Id-D6 |
| Structure | 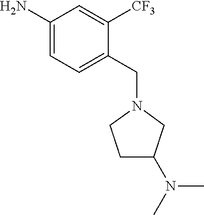 [SMILES: CN(C)C1CCN(Cc2ccc(N)cc2C(F)(F)F)C1] | 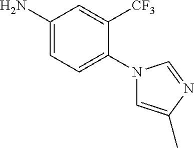 [SMILES: Cc1cn(-c2ccc(N)cc2C(F)(F)F)cn1] | 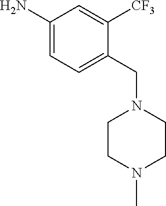 |
| Intermediate | Id-D7 | Id-D8 | ld-D9 |
| Structure | 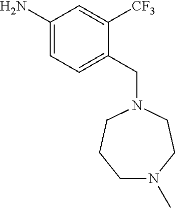 [SMILES: CN1CCCN(Cc2ccc(N)cc2C(F)(F)F)CC1] | 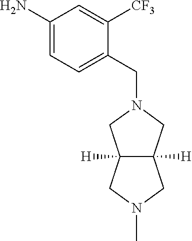 [SMILES: CN1C[C@@H]2CN(Cc3ccc(N)cc3C(F)(F)F)C[C@@H]2C1] | 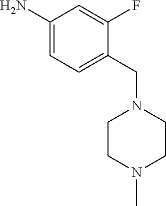 |
| Intermediate | Id-D10 | Id-D11 | Id-D12 |
| Structure | 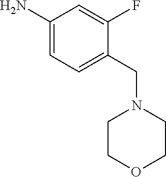 [SMILES: Nc1ccc(CN2CCOCC2)c(F)c1] | 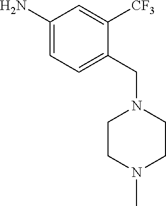 [SMILES: CN1CCN(Cc2ccc(N)cc2C(F)(F)F)CC1] | 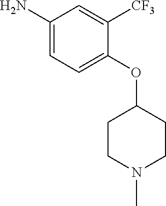 |

All amine Intermediates Id-D are commercially available except for the following:

Intermediate Id-D3 was prepared as reported in PCT Publication No. WO 2018/215668 A1.

Intermediate Id-D4 was prepared as reported in PCT Publication No. WO 2020/206583 A1.

Intermediate Id-D5 was prepared as reported in PCT Publication No. WO 2016/029776 A1.

Intermediate Id-D7 was prepared as reported in PCT Publication No WO 2017/222285 A1.

Intermediate Id-D8 was prepared as reported in, J. Med. Chem. 2012, 55(22), 10033-10046.

Intermediate Id-D9 was prepared as reported in PCT Publication No. WO 2020/135507 A1.

Intermediate Id-D10 was prepared as reported in PCT Publication No WO 2014/012360 A1.

Intermediate Id-D11 was prepared as reported in PCT Publication No. WO 2008/046802 A1.

Intermediate Id-D12 was prepared as reported in PCT Publication No. WO 2019/200120 A1.

### Preparation of Examples

### General Urea Formation Procedure for the Synthesis of Examples Id-1 Through Id-25.

Method Id-A—DMAP (0.05 eq.) and DIPEA (1.5 eq.) were added to a solution of carbamate Intermediate Id-C (1.0 eq.) and amine Intermediate Id-D (1.0 eq.) in THF (10 vol) and the resulting mixture was stirred at 60° C. for 12 h in a sealed tube. Following completion of the reaction (as indicated by LCMS), the reaction mixture was concentrated under reduced pressure to yield crude material which was purified by reverse phase preparative HPLC, affording the title product.

Method Id-B—Triethylamine (2.0 eq.) was added to a mixture of carbamate Intermediate Id-C (1.0 eq.) and amine Intermediate Id-D (1.0 eq.) in THF (5 mL) and the resulting mixture was stirred at 60° C. for 12 h in a sealed tube. Following completion of the reaction (as indicated by LCMS), the reaction mixture was concentrated under reduced pressure to yield crude material which was purified by reverse phase preparative HPLC, affording the title product.

### Example ID-1

### 1-(4-(4-amino-1-isopropyl-1H-pyrazolo[3,4-J]pyrimidin-3-yl)phenyl)-3-(3-methyl-4-morpholinophenyl)urea

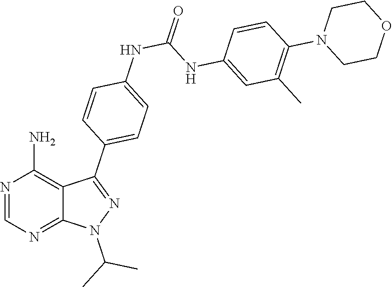 [SMILES: Cc1cc(NC(=O)Nc2ccc(-c3nn(C(C)C)c4ncnc(N)c34)cc2)ccc1N1CCOCC1]

The title compound was obtained following the general procedure for urea formation (Method Id-B), starting from 3-(4-aminophenyl)-1-isopropyl-1H-pyrazolo[3,4-d]pyrimidin-4-amine (Id-B2, 0.100 g, 0.372 mmol) and phenyl (3-methyl-4-morpholinophenyl)carbamate (Id-C4, 0.116 g, 0.372 mmol), and was obtained as an off-white solid (0.023 g, 13% yield). ¹H NMR (400 MHz, DMSO-d₆) δ=8.84 (bs, 1H), 8.54 (bs, 1H), 8.24 (bs, 1H), 7.56-7.64 (m, 4H), 7.26-7.29 (m, 2H), 6.99 (d, J=8.4 Hz, 1H), 5.04-5.08 (m, 1H), 3.73-3.74 (m, 4H), 2.79-2.80 (m, 4H), 2.26 (s, 3H), 1.49 (d, J=6.6 Hz, 6H). LCMS: 487.2 [M+H].

### Example ID-2

### 1-(4-(4-amino-1-(oxetan-3-yl)-1H-pyrazolo[3,4-d]pyrimidin-3-yl)phenyl)-3-(3-methyl-4-Morpholinophenyl)urea

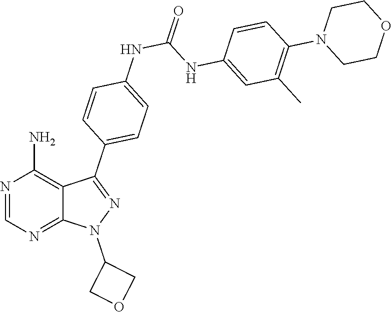 [SMILES: Cc1cc(NC(=O)Nc2ccc(-c3nn(C4COC4)c4ncnc(N)c34)cc2)ccc1N1CCOCC1]

The title compound was obtained following the general procedure for urea formation (Method Id-B), starting from 3-(4-aminophenyl)-1-(oxetan-3-yl)-1H-pyrazolo[3,4-d]pyrimidin-4-amine (Id-B3, 0.090 g, 0.318 mmol) and phenyl (3-methyl-4-morpholinophenyl)carbamate (Id-C4, 0.099 g, 0.318 mmol), and was obtained as an off-white solid (0.015 g, 10% yield). ¹H NMR (400 MHz, DMSO-d₆) δ=9.04 (bs, 1H), 8.72 (bs, 1H), 8.25 (bs, 1H), 7.62-7.68 (m, 4H), 7.26-7.30 (m, 2H), 6.99 (d, J=8.5 Hz, 1H), 5.98-6.05 (m, 1H), 5.10 (t, J=6.4 Hz, 2H), 4.99 (t, J=6.8 Hz, 2H), 3.72-3.74 (m, 4H), 2.78-2.80 (m, 4H), 2.26 (s, 3H). LCMS: 501.2 [M+H].

### Example ID-3

### 1-(4-(4-amino-1-isopropyl-1H-pyrazolo[3,4-d]pyrimidin-3-yl)phenyl)-3-(4-((4-methylpiperazin-1-yl)methyl)-3-(trifluoromethyl)phenyl)urea

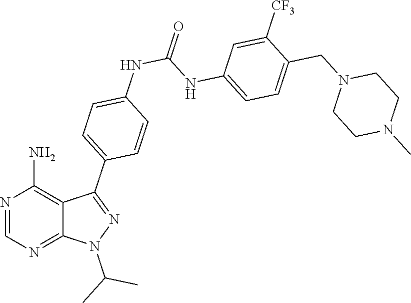 [SMILES: CC(C)n1nc(-c2ccc(NC(=O)Nc3ccc(CN4CCN(C)CC4)c(C(F)(F)F)c3)cc2)c2c(N)ncnc21]

The title compound was obtained following the general procedure for urea formation (Method Id-B), starting from 3-(4-aminophenyl)-1-isopropyl-1H-pyrazolo[3,4-d]pyrimidin-4-amine (Id-B2, 0.080 g, 0.298 mmol) and phenyl (44(4-methylpiperazin-1-yl)methyl)-3-(trifluoromethyl)phenyl)carbamate (Id-C1, 0.117 g, 0.298 mmol), and was obtained as an off-white solid (0.038 g, 22% yield). ¹H NMR (400 MHz, DMSO-d₆) δ=9.33 (bs, 1H), 9.26 (bs, 1H), 8.24 (s, 1H), 8.01 (s, 1H), 7.58-7.68 (m, 6H), 5.03-5.08 (m, 1H), 3.54 (s, 2H), 2.34-2.39 (m, 81H), 2.17 (s, 3H), 1.50 (d, J=6.8 Hz, 6H). LCMS: 568.2[M+H].

### Example ID-4

### 1-(4-(4-amino-1-(oxetan-3-yl)-1H-pyrazolo[3,4-d]pyrimidin-3-yl)phenyl)-3-(3-(4-methyl-1H-imidazol-1-yl)-5-(trifluoromethyl)phenyl)urea

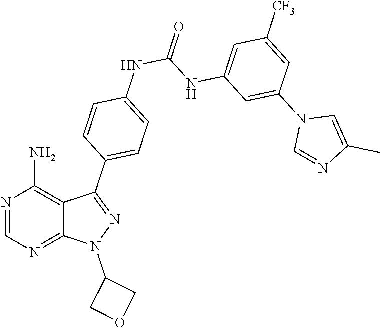 [SMILES: Cc1cn(-c2cc(NC(=O)Nc3ccc(-c4nn(C5COC5)c5ncnc(N)c45)cc3)cc(C(F)(F)F)c2)cn1]

The title compound was obtained following the general procedure for urea formation (Method Id-B), starting from 3-(4-aminophenyl)-1-(oxetan-3-yl)-1H-pyrazolo[3,4-d]pyrimidin-4-amine (Id-B3, 0.080 g, 0.22 mmol) and phenyl (3-(4-methyl-1H-imidazol-1-yl)-5-(trifluoromethyl)phenyl)carbamate (Id-C3, 0.062 g, 0.22 mmol), and was obtained as an off-white solid (0.020 g, 16% yield). ¹H NMR (400 MHz, DMSO-d₆) δ=9.54 (bs, 1H), 9.50 (bs, 1H), 8.26 (s, 1H), 8.22 (bs, 1H), 7.90 (d, J=6.0 Hz, 2H), 7.66-7.73 (m, 4H), 7.59 (s, 1H), 7.50 (s, 1H), 6.00-6.04 (m, 1H), 5.11 (t, J=6.4 Hz, 2H), 5.00 (t, J=6.8 Hz, 2H), 2.19 (s, 3H). LCMS: 549.9[M+H].

### Example ID-5

### 1-(4-(4-amino-1-cyclopropyl-1H-pyrazolo[3,4-d]pyrimidin-3-yl)-2-fluorophenyl)-3-(4-((4-methylpiperazin-1-yl)methyl)-3-(trifluoromethyl)phenyl)urea

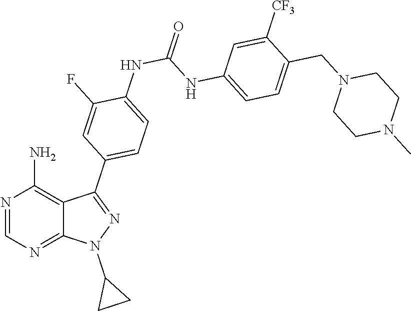 [SMILES: CN1CCN(Cc2ccc(NC(=O)Nc3ccc(-c4nn(C5CC5)c5ncnc(N)c45)cc3F)cc2C(F)(F)F)CC1]

The title compound was obtained following the general procedure for urea formation (Method Id-A), starting from 3-(4-amino-3-fluorophenyl)-1-cyclopropyl-1H-pyrazolo[3,4-d]pyrimidin-4-anine (Id-B1, 0.100 g, 0.352 mmol) and phenyl (4-((4-methylpiperazin-1-yl)methyl)-3-(trifluoromethyl)phenyl)carbamate (Id-C1, 0.208 g, 0.528 mmol), and was obtained as an off-white solid (0.019 g, 8% yield). ¹H NMR (400 MHz, CD₃OD) δ=8.41 (s, 1H), 8.34-8.38 (m, 1H), 7.96-7.96 (m, 1H), 7.68-7.91 (m, 2H), 7.48-7.55 (m, 2H), 3.98-4.01 (m, 1H), 3.76 (s, 2H), 3.50-3.52 (m, 2H), 3.11-3.15 (m, 4H), 2.93 (s, 3H), 2.48 (bs, 2H), 1.36-1.40 (m, 2H), 1.20-1.25 (m, 2H). LCMS: 584.2 [M+H].

### Example ID-6

### 1-(4-(4-amino-1-cyclopropyl-1H-pyrazolo[3,4-d]pyrimidin-3-yl)-2-fluorophenyl)-3-(4-(4-methylpiperazine-1-carbonyl)-3-(trifluoromethyl)phenyl)urea

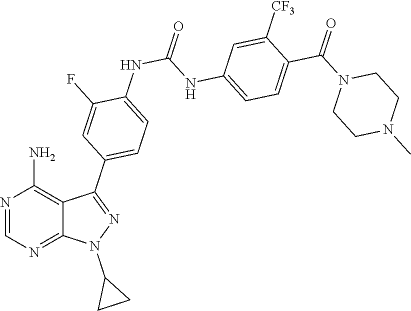 [SMILES: CN1CCN(C(=O)c2ccc(NC(=O)Nc3ccc(-c4nn(C5CC5)c5ncnc(N)c45)cc3F)cc2C(F)(F)F)CC1]

The title compound was obtained following the general procedure for urea formation (Method Id-A), starting from phenyl (4-(4-amino-1-cyclopropyl-1H-pyrazolo[3,4-d]pyrimidin-3-yl)-2-fluorophenyl)carbamate (Id-C5, 0.090 g, 0.223 mmol) and (4-amino-2-(trifluoromethyl)phenyl(4-methylpiperazin-1-yl)methanone (Id-D2, 0.064 g, 0.223 mmol), and was obtained as an off-white solid (0.008 g, 6% yield). ¹H NMR (400 MHz, CD₃OD) δ=8.33-8.37 (m, 1H), 8.30 (s, 1H), 8.08 (d, J=2.0 Hz, 1H), 7.75-7.78 (m, 1H), 7.48-7.53 (m, 2H), 7.39-7.41 (m, 1H), 3.81-3.85 (m, 3H), 3.24-3.28 (m, 2H), 2.53-2.61 (m, 2H), 2.42 (s, 2H), 2.37 (s, 3H), 1.29-1.33 (m, 2H), 1.16-1.21 (m, 2H) LCMS: 598.4 [M+H].

### Example ID-7

### 1-(4-(4-amino-t-cyclopropyl-1H-pyrazolo[3,4-d]pyrimidin-3-yl)-2-fluorophenyl)-3-(4-((4-ethylpiperazin-1-yl)methyl)-3-(trifluoromethyl)phenyl)urea

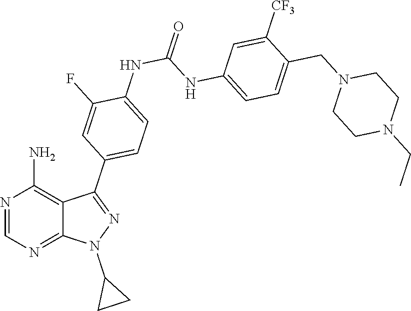 [SMILES: CCN1CCN(Cc2ccc(NC(=O)Nc3ccc(-c4nn(C5CC5)c5ncnc(N)c45)cc3F)cc2C(F)(F)F)CC1]

The title compound was obtained following the general procedure for urea formation (Method Id-A), starting from phenyl (4-(4-amino-1-cyclopropyl-1H-pyrazolo[3,4-d]pyrimidin-3-yl)-2-fluorophenyl)carbamate (Id-C5, 0.098 g, 0.213 mmol) and 4-((4-ethylpiperazin-1-yl)methyl)-3-(trifluoromethyl)aniline (Id-D1, 0.067 g, 0.235 mmol), and was obtained as an off-white solid (0.037 g, 28% yield). ¹H NMR (400 MHz, CD₃OD) δ=8.39 (s, 1H), 8.33-8.37 (m, 1H), 7.95-7.95 (m, 1H), 7.68-7.74 (m, 2H), 7.48-7.55 (m, 2H), 3.95-3.99 (m, 1H), 3.77 (bs, 2H), 3.35 (bs, 2H), 3.28-3.29 (m, 2H), 3.14-3.16 (m, 4H), 2.52 (bs, 2H), 1.35-1.39 (m, 5H), 1.20-1.24 (m, 2H). LCMS: 598.2 [M+H].

### Example ID-8

### 1-(4-(4-amino-1-cyclopropyl-1H-pyrazolo[3,4-d]pyrimidin-3-yl)-2-fluorophenyl)-3-(3-fluoro-4-(morpholinomethyl)phenyl)urea

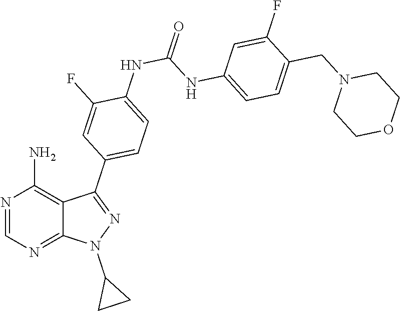 [SMILES: Nc1ncnc2c1c(-c1ccc(NC(=O)Nc3ccc(CN4CCOCC4)c(F)c3)c(F)c1)nn2C1CC1]

The title compound was obtained following the general procedure for urea formation (Method Id-A), starting from phenyl (4-(4-amino-1-cyclopropyl-1H-pyrazolo[3,4-d]pyrimidin-3-yl)-2-fluorophenyl)carbamate (Id-C5, 0.140 g, 0.305 mmol) and 3-fluoro-4-(morpholinomethyl)aniline (Id-D10, 0.064 g, 0.305 mmol), and was obtained as an off-white solid (0.018 g, 11% yield). ¹H NMR (400 MHz, CD₃OD) δ=8.32-8.36 (m, 1H), 8.29 (s, 1H), 7.46-7.55 (m, 3H), 7.34-7.38 (m, 1H), 7.13-7.16 (m, 1H), 3.79-3.84 (m, 1H), 3.72-3.75 (m, 4H), 3.68 (s, 2H), 2.61-2.63 (m, 4H), 1.29-1.33 (m, 2H), 1.17-1.21 (m, 2H). LCMS: 521.2 [M+H].

### Example ID-9

### 1-(4-(4-amino-1-(2-hydroxy-2-methylpropyl)-1H-pyrazolo[3,4-d]pyrimidin-3-yl)-2-fluorophenyl)-3-(4-((4-methylpiperazin-1-yl)methyl)-3-(trifluoromethyl)phenyl)urea

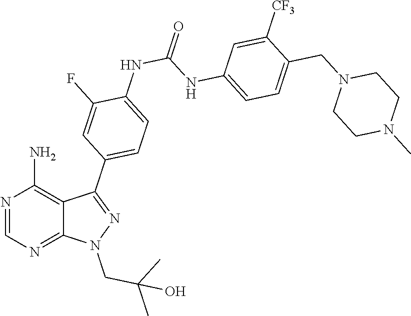 [SMILES: CN1CCN(Cc2ccc(NC(=O)Nc3ccc(-c4nn(CC(C)(C)O)c5ncnc(N)c45)cc3F)cc2C(F)(F)F)CC1]

The title compound was obtained following the general procedure for urea formation (Method Id-A), starting from 1-(4-amino-3-(4-amino-3-fluorophenyl)-1H-pyrazolo[3,4-d]pyrimidin-1-yl)-2-methylpropan-2-ol (Id-B4, 0.100 g, 0.316 mmol) and phenyl (4-((4-methylpiperazin-1-yl)methyl)-3-(trifluoromethyl)phenyl)carbamate (Id-C1, 0.187 g, 0.474 mmol), and was obtained as an off-white solid (0.010 g, 5% yield) ¹H NMR (400 MHz, CD₃OD) δ=8.37-8.40 (m, 2H), 7.96 (d, J=2.0 Hz, 1H), 7.70-7.72 (m, 2H), 7.56-7.59 (m, 2H), 4.48 (s, 2H), 3.76 (s, 2H), 3.52 (bs, 2H), 2.99-3.24 (m, 4H), 2.93 (s, 3H), 2.50 (bs, 2H), 1.32 (s, 6H). LCMS: 615.8 [M+H].

### Example ID-10

### 1-(4-(4-amino-7-cyclopropyl-7H-pyrrolo[2,3-d]pyrimidin-5-yl)-2-fluorophenyl)-3-(4-((4-methylpiperazin-1-yl)methyl)-3-(trifluoromethyl)phenyl)urea

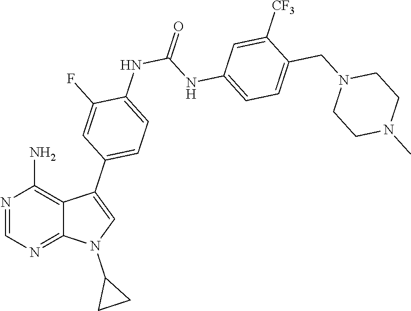 [SMILES: CN1CCN(Cc2ccc(NC(=O)Nc3ccc(-c4cn(C5CC5)c5ncnc(N)c45)cc3F)cc2C(F)(F)F)CC1]

The title compound was obtained following the general procedure for urea formation (Method Id-A), starting from 5-(4-amino-3-fluorophenyl)-7-cyclopropyl-7H-pyrrolo[2,3-d]pyrimidin-4-amine (Id-B5, 0.100 g, 0.353 mmol) and phenyl (4-((4-methylpiperazin-1-yl)methyl)-3-(trifluoromethyl)phenyl)carbamate (Id-C1, 0.139 g, 0.353 mmol), and was obtained as an off-white solid (0.039 g, 20% yield). ¹H NMR (400 MHz, CD₃OD) δ=8.36 (s, 1H), 8.21 (t, J=8.0 Hz, 1H), 7.96 (d, J=2.0 Hz, 1H), 7.67-7.73 (m, 2H), 7.47 (s, 1H), 7.29-7.37 (m, 2H), 3.72 (s, 2H), 3.67-3.71 (m, 1H), 3.49-3.51 (m, 2H), 3.04-3.18 (m, 4H), 2.93 (s, 3H), 2.48 (s, 2H), 1.15-1.25 (m, 4H). LCMS: 583.3 [M+H].

### Example ID-11

### 1-(4-(4-amino-7-cyclopropyl-7H-pyrrolo[2,3-d]pyrimidin-5-yl)-2-fluorophenyl)-3-(4-(4-methylpiperazin-1-yl)-3-(trifluoromethyl)phenyl)UR EA

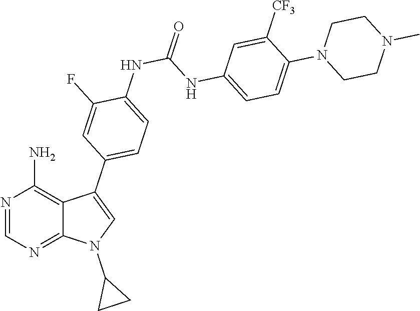 [SMILES: CN1CCN(c2ccc(NC(=O)Nc3ccc(-c4cn(C5CC5)c5ncnc(N)c45)cc3F)cc2C(F)(F)F)CC1]

The title compound was obtained following the general procedure for urea formation (Method Id-A), starting from 5-(4-amino-3-fluorophenyl)-7-cyclopropyl-7H-pyrrolo[2,3-d]pyrimidin-4-amine (Id-B5, 0.100 g, 0.353 mmol) and phenyl (4-(4-methylpiperazin-1-yl)-3-(trifluoromethyl)phenyl)carbamate (Id-C7, 0.201 g, 0.529 mmol), and was obtained as an off-white solid (0.027 g, 13% yield). ¹H NMR (400 MHz, CD₃OD) δ=8.34 (s, 1H), 8.20 (t, J=8.4 Hz, 1H), 7.95 (d, J=2.4 Hz, 1H), 7.72-7.75 (m, 1H), 7.54 (d, J=8.4 Hz, 1H), 7.44 (s, 1H), 7.29-7.36 (m, 2H), 3.62-3.77 (m, 3H), 3.19-3.24 (m, 6H), 3.01 (s, 3H), 1.17-1.21 (m, 4H). LCMS: 569.3 [M+H].

### Example ID-12

### 1-(4-(4-amino-7-methyl-7H-pyrrolo[2,3-d]pyrimidin-5-yl)-2-fluorophenyl)-3-(4-((4-methylpiperazin-1-yl)methyl)-3-(trifluoromethyl)phenyl)urea

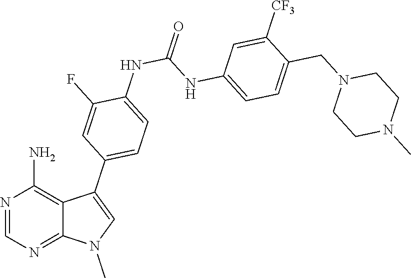 [SMILES: CN1CCN(Cc2ccc(NC(=O)Nc3ccc(-c4cn(C)c5ncnc(N)c45)cc3F)cc2C(F)(F)F)CC1]

The title compound was obtained following the general procedure for urea formation (Method Id-A), starting from phenyl (4-(4-amino-7-methyl-7H-pyrrolo[2,3-d]pyrimidin-5-yl)-2-fluorophenyl)carbamate (Id-C8, 0.060 g, 0.159 mmol) and 4-((4-methylpiperazin-1-yl)methyl)-3-(trifluoromethyl)aniline (Id-D6, 0.048 g, 0.175 mmol), and was obtained as white solid (0.009 g, 8% yield). ¹H NMR (400 MHz, DMSO-d₆) δ=9.41 (bs, 1H), 8.70 (d, J=2.4 Hz, 1H), 8.11-8.23 (m, 2H), 8.01 (d, J=2.0 Hz, 1H), 7.64-7.66 (m, 1H), 7.54-7.57 (m, 1H), 7.34 (s, 1H), 7.23-7.33 (m, 2H), 6.16 (bs, 2H), 3.74 (s, 3H), 3.55 (s, 2H), 2.41 (bs, 8H), 2.20 (s, 3H). LCMS: 556.8 [M+H].

### Example ID-13

### 1-(4-(4-amino-7-cyclobutyl-7H-pyrrolo[2,3-d]pyrimidin-5-yl)-2-fluorophenyl)-3-(4-((4-methylpiperazin-1-yl)methyl)-3-(trifluoromethyl)phenyl)urea

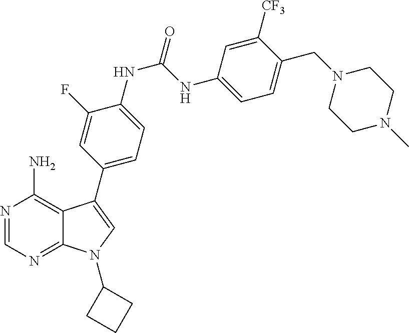 [SMILES: CN1CCN(Cc2ccc(NC(=O)Nc3ccc(-c4cn(C5CCC5)c5ncnc(N)c45)cc3F)cc2C(F)(F)F)CC1]

The title compound was obtained following the general procedure for urea formation (Method Id-A), starting from 5-(4-amino-3-trifluorophenyl)-7-cyclobutyl-7H-pyrrolo[2,3-d]pyrimidin-4-amine (Id-B7, 0.135 g, 0.454 mmol) and phenyl (4-((4-methylpiperazin-1-yl)methyl)-3-(trifluoromethyl)phenyl)carbamate (Id-C1, 0.179 g, 0.454 mmol), and was obtained as a white solid (0.043 g, 15%). ¹H NMR (400 MHz, DMSO-d₆) δ=9.53 (bs, 1H), 8.83 (bs, 1H), 8.40 (s, 1H), 8.25 (t, J=8.4 Hz, 1H), 8.02 (d, J=1.6 Hz, 1H), 7.94 (s, 1H), 7.60-7.66 (m, 2H), 7.39-7.42 (m, 1H), 7.30 (d, J=8.4 Hz, 1H), 5.24-5.26 (m, 1H), 3.88 (s, 2H), 3.40-3.43 (m, 2H), 2.90-3.03 (m, 4H), 2.82 (s, 3H), 2.56-2.68 (m, 2H), 2.40-2.46 (m, 4H), 1.85-1.90 (m, 2H) LCMS: 597.2 [M+H].

### Example ID-14

### 1-(4-(4-amino-7-cyclopropyl-7H-pyrrolo[2,3-d]pyrimidin-5-yl)-2-fluorophenyl)-3-(4-(4-methylpiperazine-1-carbonyl)-3-(trifluoromethyl)phenyl)urea

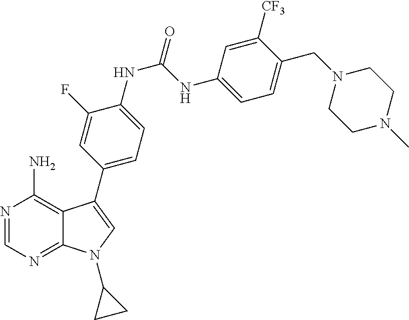 [SMILES: CN1CCN(Cc2ccc(NC(=O)Nc3ccc(-c4cn(C5CC5)c5ncnc(N)c45)cc3F)cc2C(F)(F)F)CC1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.100 g, 0.248 mmol) and (4-amino-2-(trifluoromethyl)phenyl)(4-methylpiperazin-1-yl)methanone (Id-D2, 0.078 g, 0.273 mmol), and was obtained as a white solid (0.009 g, 6% yield). ¹H NMR (400 MHz, CD₃OD) δ=8.36 (s, 1H), 8.17-8.21 (m, 1H), 8.11 (d, J=1.6 Hz, 1H), 7.79 (d, J=8.4 Hz, 11H), 7.48-7.50 (m, 2H), 7.30-7.37 (m, 2H), 3.68-3.72 (m, 1H), 3.46 (s, 8H), 2.98 (s, 3H), 1.18-1.22 (m, 4H). LCMS: 597.3 [M+H].

### Example ID-15

### 1-(4-(4-amino-7-cyclopropyl-7H-pyrrolo[2,3-d]pyrimidin-5-yl)-2-fluorophenyl)-3-(4-((4-ethylpiperazin-1-yl)methyl)-3-(trifluoromethyl)phenyl)urea

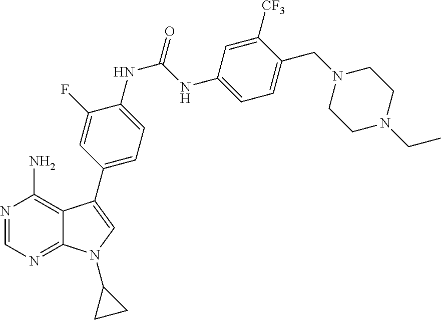 [SMILES: CCN1CCN(Cc2ccc(NC(=O)Nc3ccc(-c4cn(C5CC5)c5ncnc(N)c45)cc3F)cc2C(F)(F)F)CC1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.168 g, 0.418 mmol) and 4-((4-ethylpiperazin-1-yl)methyl)-3-(trifluoromethyl)aniline (Id-D1, 0.120 g, 0.418 mmol), and was obtained as an off-white solid (0.009 g, 4% yield). ¹H NMR (400 MHz, CD₃OD) δ=8.36 (s, 1H), 8.21 (t, J=8.0 Hz, 1H), 7.96 (d, J=2.0 Hz, 1H), 7.66-7.73 (m, 2H), 7.48 (s, 1H), 7.29-7.37 (m, 2H), 3.73 (s, 2H), 3.69-3.72 (m, 1H), 3.50-3.57 (m, 2H), 3.21-3.27 (m, 2H), 3.09-3.16 (m, 4H), 2.49 (bs, 2H), 1.37 (t, J=7.2 Hz, 3H), 1.18-1.22 (m, 4H). LCMS: 597.3 [M+H].

### Example ID-16

### 1-(4-(4-amino-7-cyclopropyl-7H-pyrrolo[2,3-d]pyrimidin-5-yl)-2-fluorophenyl)-3-(4-((4-methyl-1,4-DIAZEPAN-1-yl)methyl)-3-(trifluoromethyl)phenyl)urea

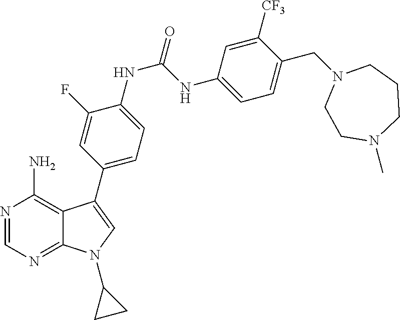 [SMILES: CN1CCCN(Cc2ccc(NC(=O)Nc3ccc(-c4cn(C5CC5)c5ncnc(N)c45)cc3F)cc2C(F)(F)F)CC1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.160 g, 0.397 mmol) and 4-((4-methyl-1,4-diazepan-1-yl)methyl)-3-(trifluorodimethyl)aniline (Id-D7, 0.080 g, 0.278 mmol), and was obtained as an off-white solid (0.010 g, 4% yield). ¹H NMR (400 MHz, CD₃OD) δ=8.15-8.21 (m, 2H), 7.90 (d, J=2.0 Hz, 1H), 7.78 (d, J=8.4 Hz, 1H), 7.69-7.72 (m, 1H), 7.27-7.33 (m, 2H), 7.21 (s, 1H), 3.84 (s, 2H), 3.51-3.54 (m, 1H), 3.35-3.38 (m, 2H), 3.24-3.28 (m, 2H), 2.79-2.91 (m, 7H), 2.04-2.07 (m, 2H), 1.13-1.17 (m, 2H), 1.07-1.10 (m, 2H). LCMS: 596.9 [M+H].

### Example ID-17

### 1-(4-(4-amino-7-cyclopropyl-7H-pyrrolo[2,3-d]pyrimidin-5-yl)-2-fluorophenyl)-3-(4-(morpholinomethyl)-3-(trifluoromethyl)phenyl)urea

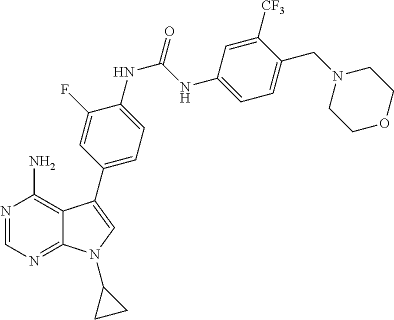 [SMILES: Nc1ncnc2c1c(-c1ccc(NC(=O)Nc3ccc(CN4CCOCC4)c(C(F)(F)F)c3)c(F)c1)cn2C1CC1]

The title compound was obtained following the general procedure for urea formation (Method Id-A), starting from phenyl (4-(4-amino)-7-cyclopropyl-7H-pyrrolo[2,3-d]pyrimidin-5-yl)-2-fluorophenyl)carbamate (Id-C₆, 0.077 g, 0.191 mmol) and 4-(morpholinomethyl)-3-(trifluoromethyl)aniline (Id-D3, 0.071 g, 0.273 mmol), and was obtained as an off-white solid (0.012 g, 7% yield). ¹H NMR (400 MHz, DMSO-d₆) δ=9.65 (bs, 1H), 8.88 (bs, 1H), 8.41 (s, 1H), 8.20-8.24 (m, 1H), 8.09 (bs, 1H), 7.70-7.78 (m, 2H), 7.70 (s, 1H), 7.35-7.39 (m, 1H), 7.24-7.28 (m, 1H), 4.35 (s, 2H), 3.67-3.72 (m, 5H), 3.20 (bs, 4H), 1.09-1.11 (m, 4H). LCMS: 570.3 [M+H].

### Example ID-18

### 1-(4-(4-amino-7-cyclopropyl-7H-pyrrolo[2,3-d]pyrimidin-5-yl)-2-fluorophenyl)-3-(4-((3-(dimethylamino)pyrrolidin-1-yl)methyl)-3-(Trifluoromethyl)phenyl)urea

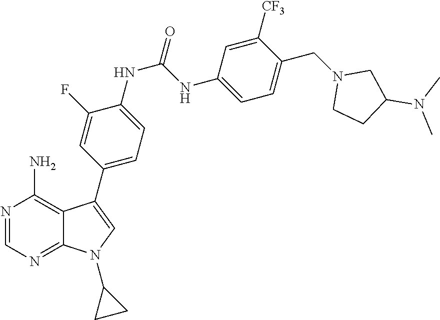 [SMILES: CN(C)C1CCN(Cc2ccc(NC(=O)Nc3ccc(-c4cn(C5CC5)c5ncnc(N)c45)cc3F)cc2C(F)(F)F)C1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.100 g, 0.193 mmol) and 1-(4-amino-2-(trifluoromethyl)benzyl)-N,N-dimethylpyrrolidin-3-amine (Id-D4, 0.061 g, 0.213 mmol), and was obtained as an off-white solid (0.040 g, 34% yield). ¹H NMR (400 MHz, CD₃OD) δ=8.37 (s, 1H), 8.21 (t, J=8.0 Hz, 1H), 8.04 (s, 1H), 7.73-7.73 (m, 21H), 7.49 (s, 1H), 7.30-7.37 (m, 2H)), 4.15-4.16 (m, 2H), 4.02-4.04 (m, 1H), 3.71-3.73 (m, 1H), 3.40-3.41 (m, 1H), 3.24-3.28 (m, 2H), 2.92-2.95 (m, 7H), 2.45-2.48 (m, 1H), 2.18-2.24 (m, 1H), 1.18-1.22 (m, 4H). LCMS: 597.3 [M+H].

### Example ID-19

### 1-(4-(4-amino-7-cyclopropyl-7H-pyrrolo[2,3-d]pyrimidin-5-yl)-2-fluorophenyl)-3-(4-(((3aR,6aS)-5-methylhexahydropyrrolo[3,4-c]pyrrol-2(1H)-yl)methyl)-3-(trifluoromethyl)phenyl)urea

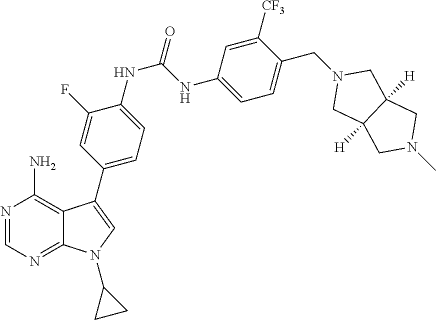 [SMILES: CN1C[C@@H]2CN(Cc3ccc(NC(=O)Nc4ccc(-c5cn(C6CC6)c6ncnc(N)c56)cc4F)cc3C(F)(F)F)C[C@@H]2C1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.075 g, 0.186 mmol) and 4-(((3aR,6aS)-5-methylhexahydropyrrolo[3,4-c]pyrrol-2(1H)-yl)methyl)-3-(trifluoromethyl)aniline (Id-D8, 0.056 g, 0.186 mmol), and was obtained as an off-white solid (0.016 g, 13% yield). ¹H NMR (400 MHz, CD₃OD) δ=8 17-8.20 (m, 2H), 7.91 (bs, 1H), 7.71-7.73 (m, 2H), 7.28-7.33 (m, 2H), 7.21 (bs, 1H), 3.79 (bs, 2H), 3.52-3.53 (m, 3H), 3.03 (bs, 4H), 2.88 (s, 3H), 2.78-2.81 (m, 2H), 2.41 (bs, 2H), 1.09-1.17 (m, 4H) LCMS: 609.3 [M+H].

### Example ID-20

### 1-(4-(4-amino-7-cyclopropyl-7H-pyrrolo[2,3-d]pyrimidin-5-yl)-2-fluorophenyl)-3-(3-fluoro-4-((4-methylpiperazin-1-yl)methyl)phenyl)urea

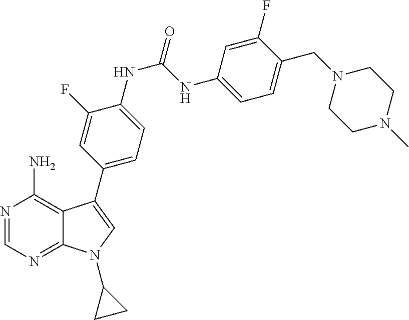 [SMILES: CN1CCN(Cc2ccc(NC(=O)Nc3ccc(-c4cn(C5CC5)c5ncnc(N)c45)cc3F)cc2F)CC1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.075 g, 0.186 mmol) and 3-fluoro-4-((4-methylpiperazin-1-yl)methyl)aniline (Id-D9, 0.042 g, 0.186 mmol), and was obtained as an off-white solid (0.005 g, 5% yield). ¹H NMR (400 MHz, CD₃OD) δ=8.20 (s, 1H), 8.14-8.18 (m, 1H), 7.49-7.53 (m, 1H), 7.27-7.36 (m, 3H), 7.21 (s, 1H), 7.13-7.16 (m, 1H), 3.70 (s, 2H), 3.50-3.55 (m, 1H), 3.15-3.19 (m, 4H), 2.80-2.87 (m, 7H), 1.06-1.18 (m, 4H). LCMS: 533.3 [M+H].

### Example ID-21

### 1-(4-(4-amino-7-cyclopropyl-7H-pyrrolo[2,3-d]pyrimidin-5-yl)-2-fluorophenyl)-3-(3-fluoro-4-(morpholinomethyl)phenyl)urea

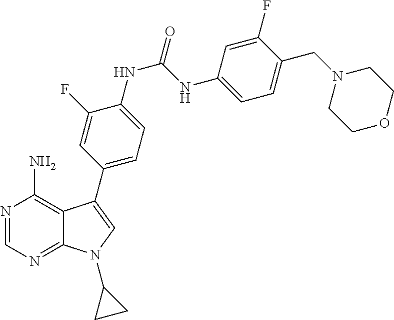 [SMILES: Nc1ncnc2c1c(-c1ccc(NC(=O)Nc3ccc(CN4CCOCC4)c(F)c3)c(F)c1)cn2C1CC1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.140 g, 0.347 mmol) and 3-fluoro-4-(morpholinomethyl)aniline (Id-D10, 0.073 g, 0.347 mmol), and was obtained as an off-white solid (0.035 g, 17% yield). ¹H NMR (400 MHz, CD₃OD) δ=8.21 (s, 1H), 8.17-8.19 (m, 1H), 7.58-7.62 (m, 1H), 7.39-7.44 (m, 1H), 7.27-7.33 (m, 2H), 7.23 (s, 1H), 7.18-7.20 (m, 1H), 3.95 (s, 2H), 3.74-3.82 (m, 4H), 3.55-3.58 (m, 1H), 2.90 (bs, 4H), 1.07-1.19 (m, 4H). LCMS: 520.2 [M+H].

### Example ID-22

### 1-(4-(4-amino-7-cyclopropyl-7H-pyrrolo[2,3-d]pyrimidin-5-yl)-2-fluorophenyl)-3-(4-((1-methylpiperidin-4-yl)methyl)-3-(trifluoromethyl)phenyl)urea

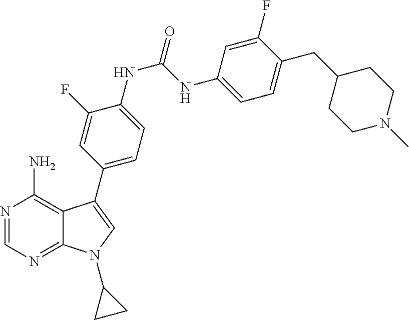 [SMILES: CN1CCC(Cc2ccc(NC(=O)Nc3ccc(-c4cn(C5CC5)c5ncnc(N)c45)cc3F)cc2F)CC1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.075 g, 0.186 mmol) and 4-((1-methylpiperidin-4-yl)methyl)-3-(trifluoromethyl)aniline (Id-D11, 0.051 g, 0.186 mmol), and was obtained as a white solid (0.040 g, 33% yield). ¹H NMR (400 MHz, CD₃OD) δ=8.30-8.34 (m, 2H), 7.94 (d, J=2.4 Hz, 1H), 7.64-7.67 (m, 1H), 7.50-7.58 (m, 2H), 7.40-7.42 (m, 1H), 6.90 (s, 1H), 3.60-3.62 (m, H), 3.51-3.54 (m, 2H), 2.94-3.00 (m, 2H), 2.86 (s, 3H), 2.78-2.79 (m, 2H), 1.92-1.95 (m, 3H), 1.55-1.58 (m, 2H), 1.12-1.14 (m, 2H), 0.80-0.83 (m, 2H). LCMS: 582.3 [M+H].

### Example ID-23

### 1-(4-(4-amino-7-cyclopropyl-7H-pyrrolo[2,3-d]pyrimidin-5-yl)-2-fluorophenyl-3-(4-(4-methyl-1H-imidazol-1-yl)-3-(trifluoromethyl)phenyl)urea

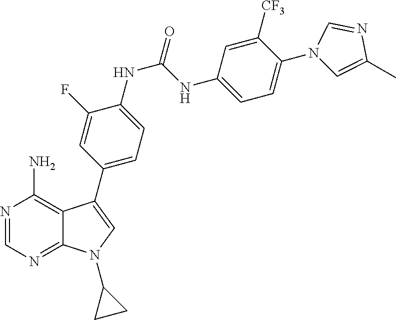 [SMILES: Cc1cn(-c2ccc(NC(=O)Nc3ccc(-c4cn(C5CC5)c5ncnc(N)c45)cc3F)cc2C(F)(F)F)cn1]

The title compound was obtained following the general procedure for urea formation (Method Id-A), starting from phenyl (4-(4-amino-7-cyclopropyl-7H-pyrrolo[2,3-d]pyrimidin-5-yl)-2-fluorophenyl)carbamate (Id-C6, 0.160 g, 0.397 mmol) and 4-(4-methyl-1H-imidazol-1-yl)-3-(trifluoromethyl)aniline (Id-D5, 0.096 g, 0.397 mmol), and was obtained as a white solid (0.010 g, 5% yield). ¹H NMR (400 MHz, CD₃OD) δ=8.20-8.22 (m, 2H), 8.15-8.15 (m, 1H), 7.79-7.82 (m, 1H), 7.66 (s, 1H), 7.45 (d, J=8.4 Hz, 1H), 7.29-7.34 (m, 2H), 7.22 (s, 1H), 7.00 (s, 1H), 3.50-3.54 (m, 1H), 2.27 (s, 3H), 1.07-1.17 (m, 4H). LCMS: 551.1 [M+H].

### Example ID-24

### 1-(4-(4-amino-7-cyclopropyl-7H-pyrrolo[2,3-d]pyrimidin-5-yl)-2-fluorophenyl)-3-(3-chloro-4-((4-mesitylpiperazine-1-yl)methyl)phenyl)urea

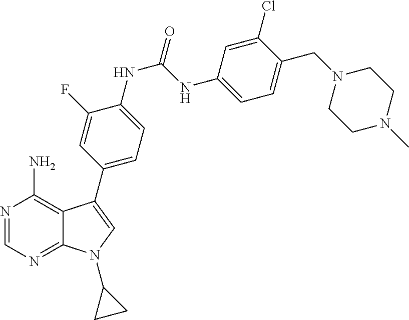 [SMILES: CN1CCN(Cc2ccc(NC(=O)Nc3ccc(-c4cn(C5CC5)c5ncnc(N)c45)cc3F)cc2Cl)CC1]

The title compound was obtained following the general procedure for urea formation (Method Id-A), starting from 5-(4-amino-3-fluorophenyl)-7-cyclopropyl-7H-pyrrolo[2,3-d]pyrimidin-4-amine (Id-B5, 0.060 g, 0.212 mmol) and phenyl (3-chloro-4-((4-methylpiperazin-1-yl)methyl)phenyl)carbamate (Id-C9, 0.084 g, 0.233 mmol), and was obtained as an off-white gum (0.010 g, 8% yield). ¹H NMR (400 MHz, CD₃OD) δ=8.37 (s, 1H), 8.22 (t, J=8.4 Hz, 1H), 7.74 (d, J=2.4 Hz, 1H), 7.49 (s, 1H), 7.43 (d, J=8.4 Hz, 1H), 7.33-7.44 (m, 2H), 7.29-7.31 (m, 1H), 3.70-3.75 (m, 3H), 2.90 (s, 3H), 2.40-2.89 (m, 8H), 1.18-1.23 (m, 4H). LCMS: 549.3 [M+H].

### Example ID-25

### 1-(4-(4-amino-7-cyclopropyl-7H-pyrrolo[2,3-d]pyrimidin-5-yl)-2-fluorophenyl)-3-(4-((1-methylpiperidin-4-yl)oxy)-3-(trifluoromethyl)phenyl)urea

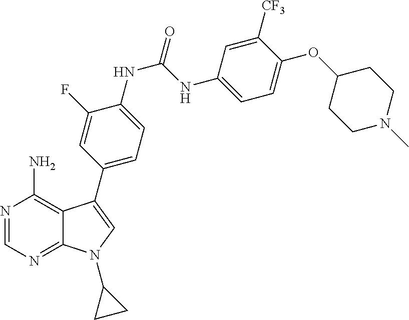 [SMILES: CN1CCC(Oc2ccc(NC(=O)Nc3ccc(-c4cn(C5CC5)c5ncnc(N)c45)cc3F)cc2C(F)(F)F)CC1]

The title compound was obtained following the general procedure for urea formation (Method Id-A), starting from phenyl (4-(4-amino-7-cyclopropyl-7H-pyrrolo[2,3-<]pyrimidin-5-yl)-2-fluorophenyl)carbamate (Id-C6, 0.150 g, 0.372 mmol) and 4-((1-methylpiperidin-4-yl)oxy)-3-(trifluoromethyl)aniline (Id-D12, 0.102 g, 0.372 mmol), and was obtained as pale brown solid (0.100 g, 44% yield). ¹H NMR (400 MHz, (CD₃OD) δ=8.36 (s, 1H), 8.19 (t, J=8.4 Hz, 1H), 7.88 (d, J=2.4 Hz, 1H), 7.63-7.66 (m, 1H), 7.48 (s, 1H), 7.24-7.36 (m, 3H), 4.96 (bs, 1H), 3.69-3.72 (m, 2H), 3.49-3.51 (m, 1H), 3.23-3.30 (m, 2H), 2.93 (bs, 3H), 1.90-2.48 (m, 4H), 1.18-1.23 (m, 4H). LCMS: 584.3 [M+H].

### Intermediate IE-A1

### 4-chloro-1-((2-(trimethylsilyl)ethoxy)methyl)-1H-pyrrolo[2,3-b]pyridine

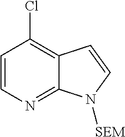 [SMILES: C[Si](C)(C)CCOCn1ccc2c(Cl)ccnc21]

### Method Ie-A

NaH (60% dispersion in mineral oil, 0.118 g, 4.92 mmol) was added in portions to a stirred solution of 4-chloro-1H-pyrrolo[2,3-b]pyridine (0.500 g, 3.28 mmol) in DMF (5 mL) at 0° C. and the resulting suspension was stirred at 0° C. for 20 min. SEM-Cl (0.697 mL, 3.93 mmol) was then added and the reaction mixture was allowed to warm to 25° C. and was stirred for 5 h. Following completion of the reaction (as indicated by TLC), the mixture was quenched with ice-cold water (25 mL) and extracted with EtOAc (2-25 mL). The combined organic phases were dried over Na₂SO₄, filtered, and concentrated under reduced pressure to give crude material which was purified by Isolera (silica gel 230-400 mesh, eluting with 4% EtOAc in petroleum ether), affording the title compound as a yellow liquid (0.77 g, 82% yield). ¹H NMR (400 MHz, DMSO-d₆) δ=8.36 (d, J=6.8 Hz, 1H), 7.89 (d, J=4.8 Hz, 1H), 7.40 (s, 1H), 6.72 (d, J=4.8 Hz, 1H), 5.75 (s, 2H), 3.62 (t, J=10.8 Hz, 2H), 0.92 (t, J=10.4 Hz, 2H), −0.011 (s, 9H) LCMS: 283.0 [M+H].

### Method Ie-B

DIPEA (11.45 mL, 65.5 mmol) and SEM-Cl (8.14 mL, 45.9 mmol) were added to a stirred solution of 4-chloro-1H-pyrrolo[2,3-b]pyridine (5.00 g, 32.8 mmol) in ACN (25 mL) at 0° C. and the resulting mixture was allowed to warm to 25° C. and was stirred for 12 h. Following completion of the reaction (as indicated by UPLC and TLC), the reaction mixture was concentrated under reduced pressure to yield crude material which was purified by Isolera (silica gel 230-400 mesh, eluting with 4% EtOAc in petroleum ether), affording the title compound as a yellow liquid (9.24 g, 99% yield). ¹H NMR (400 MHz, DMSO-d₆) δ=8.36 (d, J=6.8 Hz, 1H), 7.89 (d, J=4.8 Hz, 1H), 7.40 (s, 1H), 6.72 (d, J=4.8 Hz, 1H), 5.75 (s, 2H), 3.62 (t, J=10.8 Hz, 2H), 0.92 (t, J=10.4 Hz, 2H), −0.011 (s, 9H). LCMS: 283.0 [M+H].

### Intermediate IE-A2

### 4-bromo-1-((2-(trimethylsilyl)ethoxy)methyl-1H-pyrrolo[2,3-b]pyridine

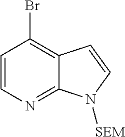 [SMILES: C[Si](C)(C)CCOCn1ccc2c(Br)ccnc21]

### Method Ie-A

NaH (60% dispersion in mineral oil, 0.609 g, 15.23 mmol) was added in portions to a stirred solution of 4-bromo-1H-pyrrolo[2,3-b]pyridine (2.000 g, 10.15 mmol) in DMF (20 mL) at 0° C. and the resulting suspension was stirred at 0° C. for 20 min. SEM-Cl (2.031 g, 12.18 mmol) was then added and the reaction mixture was allowed to warm to 25° C. and was stirred for 3 h. Following completion of the reaction (as indicated by TLC), the mixture was quenched with ice-cold water (25 mL) and extracted with EtOAc (2.25 mL). The combined organic phases were dried over Na₂SO₄, filtered, and concentrated under reduced pressure to give crude material which was purified by Isolera (silica gel 230-400 mesh, eluting with 4% EtOAc in petroleum ether), affording the title compound as a colorless liquid (2.640 g, 79% yield). ¹H NMR (400 MHz, DMSO-d₆) δ=8.16 (d, J=6.8 Hz, 1H), 7.80 (d, J=4.80 Hz, 1H), 7.43-7.44 (m, 1H), 6.52-6.53 (m, 1H), 5.64 (bs, 2H), 3.51 (t, J=10.8 Hz, 2H), 0.81 (t, J=10.4 Hz, 2H), −0.11 (s, 9H). LCMS: 327.0 [M+H].

### Method Ie-B

DIPEA (0.886 mL, 5.08 mmol) and SEM-Cl (0.630 ml., 3.55 mmol) were added to a stirred solution of 4-bromo-1H-pyrrolo[2,3-b]pyridine (0.500 g, 2.54 mmol) in ACN (10 mL) at 0° C. and the resulting mixture was allowed to warm to 25° C. and was stirred for 12 h. Following completion of the reaction (as indicated by UPLC and TLC), the reaction mixture was concentrated under reduced pressure to yield crude material which was purified by Isolera (silica gel 230-400 mesh, eluting with 4% EtOAc in petroleum ether), affording the title compound as a colorless liquid (0.680 g, 81% yield) 1H NMR (400 MHz, DMSO-d₆) δ=8.16 (d, J=6.8 Hz, 1H), 7.80 (d, J=4.8 Hz, 1H), 7.43-7.44 (m, 1H), 6.52-6.53 (m, 1H), 5.64 (bs, 2H), 3.51 (t, J=10.8 Hz, 2H), 0.81 (t, J=10.4 Hz, 2H), −0.11 (s, 9H). LCMS: 327.0 [M+H].

### Intermediate IE-A3

### 4-chloro-2-methyl-1-((2-(trimethylsilyl)ethoxy)methyl)-1H-pyrrolo[2,3-b]pyridine

### Step 1: Synthesis of 4-chloro-1-(phenylsulfonyl)-1H-pyrrolo[2,3-b]pyridine

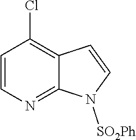

DMAP (0.080 g, 0.655 mmol) and benzenesulfonyl chloride (1.389 g, 7.860 mol) were added to a solution of 4-chloro-1H-pyrrolo[2,3-b]pyridine (1.000 g, 6.550 mmol) in DCM (15 mL) at 0° C. and the resulting mixture was stirred at 25° C. for 15 h. Following completion of the reaction (as indicated by TLC), the reaction mixture was diluted with DCM (30 mL) and washed with 1 M HCl (20 mL), aqueous NaHCO₃(20 mL), and brine (20 mL). The organic layer was dried over Na₂SO₄, filtered, and concentrated under reduced pressure to yield crude material which was purified by flash chromatography (silica gel 230-400 mesh, eluting with 5% EtOAc in petroleum ether), giving the title product as a white solid (1.2 g, 62% yield). ¹H NMR (400 MHz, DMSO-dr) δ=8.34 (d, J=5.2 Hz, 1H), 8.12-8.15 (m, 2H), 8.05 (d, J=4.0 Hz, 1H), 7.71-7.75 (m, 1H), 7.61-7.65 (m, 2H), 7.45 (d, 5.2 Hz, 1H), 6.87 (d, J=4.0 Hz, 1H). LCMS: 293.0 [M+H].

### Step 2: Synthesis of 4-chloro-2-methyl-1-phenylsulfonyl)-1H-pyrrolo[2,3-b]pyridine

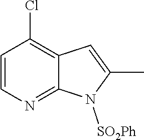

LDA (2M in THF, 3.42 ml, 6.83 mmol) was added under N2 atmosphere to a solution of 4-chloro-1-(phenylsulfonyl)-1H-pyrrolo[2,3-b]pyridine (1.00 g, 3.42 mmol) in dry THF (15 mL) at −78° C. in a 50 mL sealed tube. The resulting mixture was stirred at −78° C. for 1 h then CH₃I (6.38 ml, 102.00 mmol) was added. The reaction mixture was allowed to slowly warm to 25° C. and was stirred for 16 h. Following completion of the reaction (as indicated by LCMS), the reaction mixture was quenched with saturated NH₄Cl (15 mL) and extracted with EtOAc (2×30 mL). The combined organic extracts were dried over Na₂SO₄, filtered, and concentrated under reduced pressure to afford the title product (1.0 g, 57% yield) as a pale-yellow gum which was used without further purification. ¹H NMR (400 MHz, DMSO-d₆) δ=8.25 (d, J=5.6 Hz, 1H), 8.11-8.13 (m, 2H), 7.70-7.74 (m, 1H), 7.60-7.64 (m, 2H), 7.40 (d, J=5.2 Hz, 1H), 6.67-6.67 (m, 1H), 2.50 (s, 3H). LCMS: 307.0 [M+H].

### Step 3: Synthesis of 4-chloro-2-methyl-1H-pyrrolo[2,3-b]pyridine

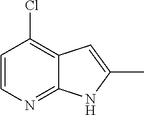

K₂CO₃ (2.253 g, 16.30 mmol) was added to a solution of 4-chloro-2-methyl-1-(phenylsulfonyl)-1H-pyrrolo[2,3-b]pyridine (1.00 g, 3.26 mmol) in a mixture of MeOH (10 mL) and water (3.33 mL) and the resulting mixture was at 60° C. for 16 h. Following completion of the reaction (as indicated by LCMS), the reaction mixture was cooled to 25° C. and the resulting precipitate was filtered, washed with water (2×5 mL), and dried to give the title product as an off white solid (0.50 g, 85% yield). ¹H NMR (400 MHz, DMSO-d₆) δ=11.86 (bs, 1H), 8.05 (d, J=6.8 Hz, 1H), 7.11 (d, J=6.8 Hz, 1H), 6.21 (d, J=1.2 Hz, 1H), 2.42 (s, 3H). LCMS: 167.1 [M+H].

### Step 4: Synthesis of 4-chloro-2-methyl-1-((2-(trimethylsilylethoxy)methyl)-1H-pyrrolo[2,3-b]pyridine

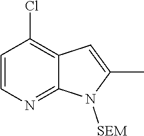 [SMILES: Cc1cc2c(Cl)ccnc2n1COCC[Si](C)(C)C]

The title compound was prepared by following a similar procedure described for Intermediate Ie-A2 (Method Ie-B), starting from 4-chloro-2-methyl-1H-pyrrolo[2,3-b]pyridine (0.750 g, 4.50 mmol). DIPEA (1.455 mL, 8.10 mmol), and SEM-Cl (1.118 ml, 6.30 mmol), and was obtained as a pale yellow liquid (0.75 g, 53% yield). ¹H NMR (400 MHz, DMSO-d₆) δ=8.39 (d, J=5.2 Hz, 1H), 7.63 (d, J=5.2 Hz, 1H), 6.99 (d, J 1.2 Hz, 1H), 4.67 (s, 2H), 3.54-3.59 (m, 2H), 2.59 (s, 3H), 0.85-0.89 (m, 2H), 0.00 (s, 9H). LCMS: 267.1 [M+H].

### Intermediate IE-A4

### 4-chloro-3-methyl-1-((2-(trimethylsilyl)ethoxy)methyl)-1H-pyrrolo[2,3-b]pyridine

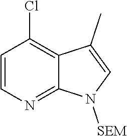 [SMILES: Cc1cn(COCC[Si](C)(C)C)c2nccc(Cl)c12]

The title compound was prepared following a similar procedure described for Intermediate Ie-A1 (Method Ie-A), starting from 4-chloro-3-methyl-1H-pyrrolo[2,3-b]pyridine (0.20 g, 0.60 mmol), NaH (60%, 0.04 g, 0.90 mmol), and SEM-Cl (0.24 g, 0.72 mmol), and was obtained as a colorless liquid (0.23 g, 90% yield). ¹H NMR (400 MHz, DMSO-d₆) δ=8.18 (d, J=5.2 Hz, 1H), 7.50 (s, 1H), 7.19 (d, J=5.2 Hz, 1H), 5.57 (s, 2H), 3.48 (t, J=8.0 Hz, 2H), 2.45 (s, 3H), 0.82 (t, J=8.0 Hz, 2H), 0.01 (s, 9H). LCMS: 297.1 [M−H].

### Intermediate IE-B1

### tert-butyl (2-fluoro-4-hydroxyphenyl)carbamate

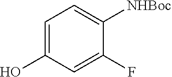 [SMILES: CC(C)(C)OC(=O)Nc1ccc(O)cc1F]

Di-tert-butyl dicarbonate (5.67 g, 26.0 mmol) was added to a mixture of 4-amino-3-fluorophenol (3.00 g, 23.6 mmol) and indium (III) chloride (0.052 g, 0.236 mmol) in DCM (30 mL) and the resulting mixture was stirred at 40° C. for 3 h. Following completion of the reaction (as indicated by TLC), the reaction mixture was quenched with water (30 mL) and extracted with EtOAc (2×10 mL). The combined organic phases were dried over Na₂SO₄., filtered, and concentrated under reduced pressure to give crude material which was purified by Isolera (eluting with 15% EtOAc in petroleum ether), affording the title compound as a brown solid (4.93 g, 91% yield). ¹H NMR (400 MHz, DMSO-d₆) δ=9.70 (bs, 1H), 8.52 (bs, 1H), 7.13-7.19 (m, 1H), 6.51-6.58 (m, 2H), 1.42 (s, 9H). LCMS: 226.1 [M−H].

### Intermediate IE-C1

### tert-butyl (2-fluoro-4-((1-((2-(trihexylsilyl)ethoxy)methyl)-1H-pyrrolo[2,3-b]pyridin-4-yl)oxy)phenyl carbamate

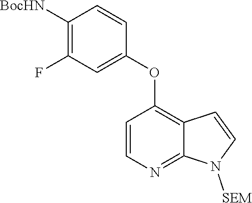 [SMILES: CC(C)(C)OC(=O)Nc1ccc(Oc2ccnc3c2ccn3COCC[Si](C)(C)C)cc1F]

### Method Ie-A

K₂CO₃ (2.452 g, 17.750 mmol) and XPhos (0.385 g, 0.807 mmol) were added to a solution of 4-bromo-1-((2-(trimethylsilyl)ethoxy)methyl)-1H-pyrrolo[2,3-b]pyridine (Ie-A2, 2.640 g, 8.070 mmol) and tert-butyl (2-fluoro-4-hydroxyphenyl)carbamate (Ie-B1, 1.943 g, 8.550 mmol) in toluene (40 mL) and the resulting suspension was purged with N₂ for 10 min. Pd₂(dba)₃ (0.369 g, 0.403 mmol) was then added and the reaction mixture was stirred at 100° C. for 12 h. Following completion of the reaction (as indicated by TLC), the reaction mixture was filtered through a pad of diatomaceous earth which was then rinsed with EtOAc (2×15 mL). The combined filtrates were concentrated under reduced pressure to yield crude material which was purified by Isolera (eluting with 20% EtOAc in petroleum ether), affording the title compound as a brown gum (2.70 g, 70% yield). ¹H NMR (400 MHz, DMSO-d₆) δ=9.70 (bs, 1H), 8.16-8.18 (m, 1H), 7.55-7.64 (m, 2H), 7.15-7.20 (m, 1H), 6.96-6.99 (m, 1H), 6.53-6.56 (m, 1H), 6.29-6.31 (m, 1H), 5.61 (s, 2H), 3.51 (t, J=10.4 Hz, 2H), 1.46 (s, 9H), 0.81 (t, J=10.8 Hz, 2H), 0.11 (s, 9H). LCMS: 474.2 [M+H].

### Method Ie-B

K₂CO₃ (1.075 g, 7.780 mmol) and XPhos (0.169 g, 0.354 mmol) were added to a solution of 4-chloro-1-((2-(trimethylsilyl)ethoxy)methyl)-1H-pyrrolo[2,3-b]pyridine (Ie-A1, 1.000 g, 3.540 mmol) and tert-butyl (2-fluoro-4-hydroxyphenyl)carbamate (Ie-B1, 0.852 g, 3.75 mmol) in toluene (20 mL) and the resulting suspension was purged with N₂ for 10 min. Pd₂(dba)₃ (0.162 g, 0.177 mmol) was then added and the reaction mixture was stirred at 100° C. for 12 h. Following completion of the reaction (as indicated by TLC), the reaction mixture was filtered through a pad of diatomaceous earth which was then rinsed with EtOAc (10 mL×2). The combined filtrates were concentrated under reduced pressure to yield crude material which was purified by Isolera (eluting with 20% EtOAc in petroleum ether), affording the title compound as a brown gum (1.30 g, 80% yield). ¹H NMR (400 MHz, DMSO-d₆) δ=9.70 (bs, 1H), 8.16-8.18 (m, 1H), 7.55-7.64 (m, 2H), 7.15-7.20 (m, 1H), 6.96-6.99 (m, 1H), 6.53-6.56 (m, 1H), 6.29-6.31 (m, 1H), 5.61 (s, 2H), 3.51 (t, J=10.4 Hz, 2H), 1.46 (s, 9H), 0.81 (t, J=10.8 Hz, 2H), 0.11 (s, 9H). LCMS: 474.2 [M+H].

### Intermediate IE-C2

### tert-butyl (4-((3-chloro-1-((2-(trimethylsilyl)ethoxy)methyl)-1H-pyrrolo[2,3-b]pyridin-4-yl)oxy)-2-fluorophenyl)carbamate

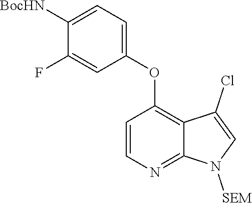 [SMILES: CC(C)(C)OC(=O)Nc1ccc(Oc2ccnc3c2c(Cl)cn3COCC[Si](C)(C)C)cc1F]

NCS (0.792 g, 5.93 mmol) was added to a solution of tert-butyl (2-fluoro-4-((1-((2-(trimethylsilyl)ethoxy)methyl)-1H-pyrrolo[2,3-b]pyridin-4-yl)oxy)phenyl)carbamate (Ie-C1, 2.700 g, 5.70 mmol) in ACN (30 mL) and the resulting mixture was stirred at 60° C. overnight. Following completion of the reaction (as indicated by UPLC), the reaction mixture was concentrated under reduced pressure to yield a residue which was taken in EtOAc (100 mL) and washed with brine (10 mL). The organic phase was dried over Na₂SO₄, filtered, and concentrated under reduced pressure to yield crude material which was purified by Isolera (eluting with 8% EtOAc in petroleum ether), affording the title compound as a colorless gum (2.20 g, 73% yield). ¹H NMR (400 MHz, DMSO-<) δ=9.04 (bs, 1H), 8.22 (d, J=5.6 Hz, 1H), 7.82 (s, 1H), 7.62-7.66 (m, 1H), 7.19-7.22 (m, 1H), 6.97-7.00 (m, 1H), 6.51 (d J=5.6 Hz, 1H), 5.61 (s, 2H), 3.54 (t, J=8.0 Hz, 2H), 1.47 (s, 9H), 0.84 (t, J=7.6 Hz, 2H), −0.08 (s, 9H) LCMS: 508.1 [M+H].

### Intermediate IE-C3

### tert-butyl (4-((2,3-dichloro-1-((2-(trimethylsilyl)ethoxy)methyl)-1H-pyrrolo[2,3-b]pyridin-4-yl)oxy)-2-fluorophenyl)carbamate

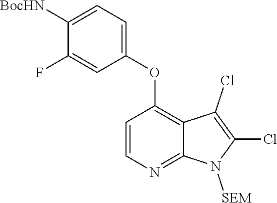 [SMILES: CC(C)(C)OC(=O)Nc1ccc(Oc2ccnc3c2c(Cl)c(Cl)n3COCC[Si](C)(C)C)cc1F]

NCS (0.806 g, 6.04 mmol) was added to a solution of tert-butyl (2-fluoro-4-((1-((2-(trimethylsilyl)ethoxy)methyl)-1H-pyrrolo[2,3-b]pyridin-4-yl)oxy)phenyl)carbamate (Ie-C1, 2.600 g, 5.49 mmol) in DMF (10 mL) at 0° C. and the resulting mixture was stirred at 25° C. for 12 h. Following completion of the reaction (as indicated by TLC and LCMS), the reaction mixture was poured into crushed ice (100 g) and extracted with EtOAc (2×50 mL). The combined organic extracts were washed with brine (25 mL), dried over Na₂SO₄, filtered, and concentrated under reduced pressure to give crude material which was purified by flash chromatography (silica gel 230-400 mesh, eluting with 10% EtOAc in petroleum ether), affording Intermediate Ie-C₃ as the minor product (colorless gum, 0.08 g, 3% yield) and Intermediate Ie-C2 as the major product (colorless gum, 2.01 g, 69% yield) LCMS: 542.1 [M+H].

### Intermediate IE-C4

### tert-butyl (4-((3-cyclopropyl-1-((2-(trimethylsilyl)ethoxy)methyl)-1H-pyrrolo[2,3-b]pyridin-4-yl)oxy)-2-fluorophenyl)carbamate

### Step 1: Synthesis of tert-butyl (4-((3-bromo-1-((2-(trimethylsilyl)ethoxy)methyl)-1H-pyrrolo[2,3-b]pyridin-4-yl)oxy)-2-fluorophenyl)carbamate

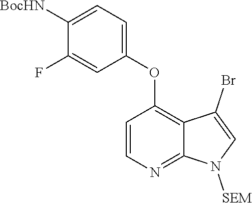 [SMILES: CC(C)(C)OC(=O)Nc1ccc(Oc2ccnc3c2c(Br)cn3COCC[Si](C)(C)C)cc1F]

NBS (0.217 g, 1.217 mmol) was added to a solution of tert-butyl (2-fluoro-4-((1-((2-(trimethylsilyl)ethoxy)methyl)-1H-pyrrolo[2,3-b]pyridin-4-yl)oxy)phenyl)carbamate (Ie-C1, 0.524 g, 1.106 mmol) in DMF (7 mL) and the resulting mixture was stirred at 25° C. overnight. Following completion of the reaction (as indicated by UPLC), the reaction mixture was poured into crushed ice (70 g), stirred at 25° C. for 15 minutes, and extracted with EtOAc (3×50 mL). The combined organic phases were washed with brine (2×10 mL), dried over Na₂SO₄, filtered, and concentrated under reduced pressure to give crude material which was purified by flash chromatography (silica gel 230-400 mesh, eluting with 30% EtOAc in petroleum ether), affording the title compound as an off-white solid (0.514 g, 59% yield). ¹H NMR (400 MHz, DMSO-d₆) δ=9.03 (bs, 1H), 8.17-8.22 (m, 1H), 7.85 (s, 1H), 7.57-7.66 (m, 1H), 7.16-7.21 (m, 1H), 6.96-6.99 (m, 1H), 6.51-6.57 (m, 1H), 5.61 (s, 2H), 3.51 (t, J=6.8 Hz, 2H), 1.47 (s, 9H), 0.86 (t, 0.1.4.8 Hz, 2H), 0.00 (s, 9H) LC-MS: 552.2 (M+H).

### Step 2: Synthesis of tert-butyl (4-((3-cyclopropyl-1-((2-(trimethylsilyl)ethoxy)methyl)-1H-pyrrolo[2,3-b]pyridin-4-yl)oxy)-2-fluorophenyl)carbamate

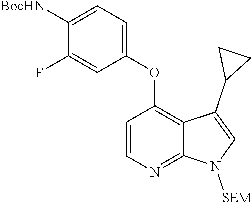 [SMILES: CC(C)(C)OC(=O)Nc1ccc(Oc2ccnc3c2c(C2CC2)cn3COCC[Si](C)(C)C)cc1F]

Potassium cyclopropyl trifluoroborane (0.074 g, 0.498 mmol), tricyclohexylphosphine (0.005 g, 0.018 mmol), Pd(OAc)₂ (0.002 g, 0.009 mmol), and K₂CO₃ (0.125 g, 0.905 mmol) were added to a solution of tert-butyl (4-((3-bromo-1-((2-(trimethylsilyl)ethoxy)methyl)-1H-pyrrolo[2,3-b]pyridin-4-yl)oxy)-2-fluorophenyl)carbamate (0.250 g, 0.452 mmol) in toluene (3 mL) and water (1 mL). The resulting suspension was degassed for 15 minutes then stirred at 90° C. in a sealed tube overnight. Following completion of the reaction (as indicated by UPLC and TLC), the reaction mixture was filtered through a pad of diatomaceous earth which was then rinsed with EtOAc. The combined filtrates were concentrated under reduced pressure to give crude material which was purified by RP-HPLC (using 0.1% TFA in water:ACN) to afford the title compound as a colorless gum (0.070 g, 27.9% Yield). LCMS: 514.2 (M+H).

### Intermediate IE-C5

### tert-butyl (4-((3-chloro-2-methyl-1-((2-(trimethylsilyl)ethoxy)methyl)-1H-pyrrolo[2,3-b]pyridin-4-yl)oxy)-2-fluorophenyl)carbamate

### Step 1: Synthesis of tert-butyl (2-fluoro-4-((2-methyl-1-((2-(trimethylsilyl)ethoxy)methyl)-1H-pyrrolo[2,3-b]pyridin-4-yl)oxy)phenyl)carbamate

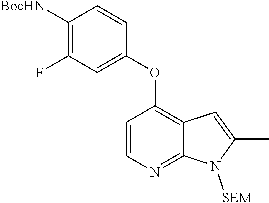 [SMILES: Cc1cc2c(Oc3ccc(NC(=O)OC(C)(C)C)c(F)c3)ccnc2n1COCC[Si](C)(C)C]

The title compound was prepared following a similar procedure described for Intermediate Ie-C1 (Method Ie-B), starting from 4-chloro-2-methyl-1-((2-(trimethylsilyl)ethoxy)methyl)-1H-pyrrolo[2,3-b]pyridine (Ie-A3, 0.700 g, 2.358 mmol), tert-butyl (2-fluoro-4-hydroxyphenyl)carbamate (Ie-B1, 0.643 g, 2.83 mmol)), K₂CO₃ (0.717 g, 5.19 mmol), XPhos (0.112 g, 0.236 mmol), and Pd₂(dba)₃ (0.108 g, 0.118 mmol), and was obtained as a brown gum (0.47 g, 38% yield). LCMS: 488.2 [M+H].

### Step 2: Synthesis of tert-butyl (4-((3-chloro-2-methyl-1-((2-(trimethylsilyl)ethoxy)methyl)-1H-pyrrolo[2,3-b]pyridin-4-yl)oxy)-2-fluorophenyl)carbamate

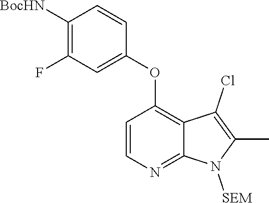 [SMILES: Cc1c(Cl)c2c(Oc3ccc(NC(=O)OC(C)(C)C)c(F)c3)ccnc2n1COCC[Si](C)(C)C]

The title compound was prepared following a similar procedure described for Intermediate Ie-C₂, starting from tert-butyl (2-fluoro-4-((2-methyl-1-((2-(trimethylsilyl)ethoxy)methyl)-1H-pyrrolo[2,3-b]pyridin-4-yl)oxy)phenyl)carbamate (0.280 g, 0.460 mmol) and NCS (0.055 g, 0.413 mmol), and was obtained as a yellow gum (0.11 g, 33% yield). LCMS: 522.2 [M+H].

### Intermediate IE-C6

### tert-butyl (2-fluoro-4-((3-methyl-1-((2-(trimethylsilyl)ethoxy)methyl)-1H-pyrrolo[2,3-H]pyridin-4-yl)oxy)phenyl)carbamate

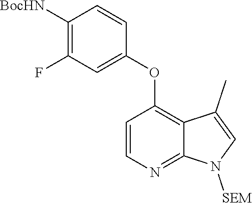 [SMILES: Cc1cn(COCC[Si](C)(C)C)c2nccc(Oc3ccc(NC(=O)OC(C)(C)C)c(F)c3)c12]

The title compound was prepared following a similar procedure described for Intermediate Ie-C1 (Method Ie-B), starting from 4-chloro-3-methyl-1-((2-(trimethylsilyl)ethoxy)methyl)-1H-pyrrolo[2,3-b]pyridine (Ie-A4, 0.230 g, 0.77 mmol), tert-butyl (2-fluoro-4-hydroxyphenyl)carbamate (Ie-B1, 0.187 g, 0.82 mmol), K₂CO₃ (0.037 g, 0.07 mmol), XPhos (0.234 g, 1.69 mmol), and Pd₂(dba)₃ (0.035 g, 0.04 mmol), and was obtained as a brown gum (0.29 g, 76% yield). ¹H NMR (400 MHz, DMSO-d₆) δ=8.99 (bs, 1H), 8.11 (d, J=5.2 Hz, 1H), 7.32 (s, 1H), 7.11-7.15 (m, 1H), 6.94 (d, J=8.8 Hz, 1H), 6.52-6.57 (m, 1H), 6.43 (d, J=5.6 Hz, 1H), 5.55 (s, 2H), 3.50 (t, J=8.0 Hz, 2H), 2.32 (s, 3H), 1.47 (s, 9H), 0.83 (t, J=8.4 Hz, 2H), −0.08 (s, 9H). LCMS: 488.1 [M+H].

### Intermediate IE-C7

### tert-butyl (4-((3-chloro-1-((2-(trimethylsilyl)ethoxy)methyl)-1H-pyrrolo[2,3-b]pyridin-4-yl)oxy)-2-methylphenyl)carbamate

### Step 1: Synthesis of tert-butyl (2-methyl-4-((1-((2-(trimethylsilyl)ethoxy)methyl)-1H-pyrrolo[2,3-b]pyridin-4-yl)oxy)phenyl)carbamate

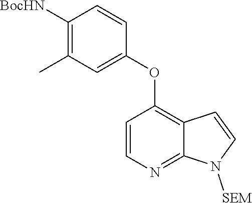 [SMILES: Cc1cc(Oc2ccnc3c2ccn3COCC[Si](C)(C)C)ccc1NC(=O)OC(C)(C)C]

The title compound was prepared by following a similar procedure described for Intermediate Ie-C1 (Method Ie-A), starting from 4-bromo-1-((2-(trimethylsilyl)ethoxy)methyl)-1H-pyrrolo[2,3-b]pyridine (Ie-A2, 0.600 g, 1.833 mmol), tert-butyl (4-hydroxy-2-methylphenyl)carbamate (0.434 g, 1.943 mmol), K₂CO₃ (0.557 g, 4.030 mmol), XPhos (0.087 g, 0.183 mmol), and Pd₂(dba)₃ (0.084 g, 0.092 mmol), and was obtained as a brown gum (0.78 g, 80% yield) LCMS: 470.3 [M+H].

### Step 2: Synthesis of tert-butyl (4-((3-chloro-1-((2-(trimethylsilyl)ethoxy)methyl)-1H-pyrrolo[2,3-b]pyridin-4-yl)oxy)-2-methylphenyl)carbamate

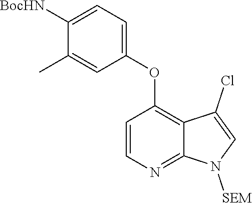 [SMILES: Cc1cc(Oc2ccnc3c2c(Cl)cn3COCC[Si](C)(C)C)ccc1NC(=O)OC(C)(C)C]

NCS (0.243 g, 1.83 mmol) was added to a solution of tert-butyl (2-methyl-4-((1-((2-(trimethylsilyl)ethoxy)methyl)-1H-pyrrolo[2,3-b]pyridin-4-yl)oxy)phenyl)carbamate (0.780 g, 1.66 mmol) in ACN (10 mL) and the resulting mixture was stirred at 25° C. for 12 h. Following completion of the reaction (as indicated by TLC), the reaction mixture was concentrated under reduced pressure to give crude material which was purified by flash chromatography (silica gel 230-400 mesh, eluting with 20% EtOAc in petroleum ether), affording the title compound as a gum (0.48 g, 58% yield). ¹H NMR (400 MHz, DMSO-d₆) δ=8.61 (bs, 1H), 8.17 (d, J=7.2 Hz, 1H), 7.79 (s, 1H), 7.40 (d, J=1.1.6 Hz, 1H), 6.97-6.98 (m, 2H), 6.40 (d, J=7.2 Hz, 1H), 5.60 (s, 2H), 3.51-3.53 (m, 2H), 2.21 (s, 3H), 1.47 (s, 9H), 0.81-0.84 (m, 2H), −0.09 (s, 9H). LCMS: 504.1 [M+H].

### Intermediate IE-C8

### tert-butyl (4-((2,3-dichloro-1-((2-(trimethylsilyl)ethoxy)methyl)-1H-pyrrolo[2,3-b]pyridin-4-yl)oxy)-2-methylphenyl)carbamate

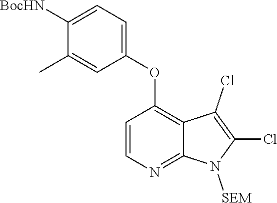 [SMILES: Cc1cc(Oc2ccnc3c2c(Cl)c(Cl)n3COCC[Si](C)(C)C)ccc1NC(=O)OC(C)(C)C]

NCS (0.140 g, 1.049 mmol) was added to a solution of tert-butyl (2-methyl-4-((1-((2-(trimethylsilyl)ethoxy)methyl)-1H-pyrrolo[2,3-b]pyridin-4-yl)oxy)phenyl)carbamate (Step 1 of Intermediate Ie-C7, 0.448 g, 0.954 mmol) in ACN (10 mL) and the resulting mixture was stirred at 60° C. for 12 h. Following completion of the reaction (as indicated by TLC), the reaction mixture was concentrated under reduced pressure, giving the crude material which was purified by flash chromatography (silica gel 230-400 mesh, eluting with 20% EtOAc in petroleum ether), affording the title compound as a colorless gum (0.30 g, 57% yield). LCMS: 538.2 [M+H].

### Intermediate IE-D1

### 4-((3-chloro-1H-pyrrolo[2,3-b]pyridin-4-yl)oxy)-2-fluoroaniline

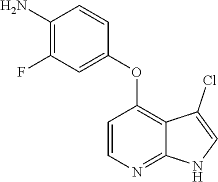 [SMILES: Nc1ccc(Oc2ccnc3[nH]cc(Cl)c23)cc1F]

TFA (10 mL, 4.33 mmol) was added to a solution of tert-butyl (4-((3-chloro-1-((2-(trimethylsilyl)ethoxy)methyl)-1H-pyrrolo[2,3-b]pyridin-4-yl)oxy)-2-fluorophenyl)carbamate (Ie-C2, 2.200 g, 4.33 mmol) in DCM (30 mL) at 0° C. and the resulting mixture was stirred at 25° C. for 12 h. Following completion of the reaction (as indicated by UPLC), the reaction mixture was concentrated under reduced pressure to give a crude residue. This brown gum was taken in MeOH (20 mL) and water (5 mL) then K₂CO₃ (1.795 g, 12.99 mmol) was added and the resulting mixture was stirred at 25° C. for 3 h. Following completion of the reaction (as indicated by UPLC), the reaction mixture was concentrated under reduced pressure to give crude material which was taken in DCM (100 mL) then washed with water (25 mL) and brine (25 mL). The organic layer was dried over Na₂SO₄, filtered, and concentrated under reduced pressure to give crude product which was purified flash chromatography (silica gel 230-400 mesh, eluting with 30% EtOAc in petroleum ether), affording the title compound as a pale brown solid (0.78 g, 59.0% yield). ¹H NMR (400 MHz, DMSO-d₆) δ=11.98 (s, 1H), 8.05-8.07 (m, 1H), 7.53-7.54 (m, 1H), 6.99-7.09 (m, 1H), 6.80-6.88 (m, 2H), 6.33-6.40 (m, 1H), 5.16 (s, 2H). LC-MS: 278.1 [M−H].

### Intermediate IE-D2

### 4-((2,3-dichloro-1H-pyrrolo[2,3-H]pyridin-4-yl)oxy)-2-fluoroaniline

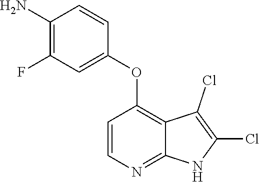 [SMILES: Nc1ccc(Oc2ccnc3[nH]c(Cl)c(Cl)c23)cc1F]

The title compound was prepared by following a similar procedure described for Intermediate Ie-D1, starting from tert-butyl (4-((2,3-dichloro-1-((2-(trimethylsilyl)ethoxy)methyl)-1H-pyrrolo[2,3-b]pyridin-4-yl)oxy)-2-fluorophenyl)carbamate (Ie-C3, 0.080 g, 0.147 mmol). TFA (1 mL), and K₂CO₃ (0.020 g, 0.147 mmol), and was obtained as a brown gum (0.040 g) which was used without further purification. LCMS: 312.0 [M+H].

### Intermediate IE-D3

### 4-((3-cyclopropyl-1H-pyrrolo[2,3-b]pyridin-4-yl)oxy)-2-fluoroaniline

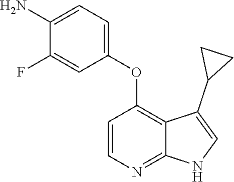 [SMILES: Nc1ccc(Oc2ccnc3[nH]cc(C4CC4)c23)cc1F]

The title compound was prepared by following a similar procedure described for Intermediate Ie-D1, starting from tert-butyl (4-((3-cyclopropyl-1-((2-(trimethylsilyl) ethoxy)methyl)-1H-pyrrolo[2,3-b]pyridin-4-yl)oxy)-2-fluorophenyl)carbamate (Ie-C4, 0.070 g, 0.136 mmol). TFA (1 mL), and K₂CO₃ (0.019 g, 0.136 mmol), and was obtained as a brown gum (0.035 g) which was used without further purification LCMS: 284.0 [M+H].

### Intermediate IE-D4

### 4-((3-chloro-2-methyl-1H-pyrrolo[2,3-b]pyridin-4-yl)oxy)-2-fluoroamine

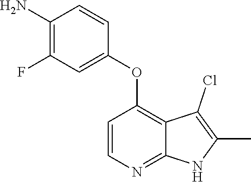 [SMILES: Cc1[nH]c2nccc(Oc3ccc(N)c(F)c3)c2c1Cl]

The title compound was prepared by following the similar procedure described for Intermediate Ie-D1, starting from tert-butyl (4-((3-chloro-2-methyl-1-((2-(trimethylsilyl)ethoxy)methyl)-1H-pyrrolo[2,3-b]pyridin-4-yl)oxy)-2-fluorophenyl)carbamate (Ie-C5, 0.110 g, 0.211 mmol), TFA (0.5 mL), and K₂CO₃ (0.088 g, 0.633 mmol), and was obtained as an off-white solid (0.07 g, 86% yield). LCMS: 292.0 [M+H].

### Intermediate IE-D5

### 2-fluoro-4-((3-methyl-1H-pyrrolo[2,3-b]pyridin-4-yl)oxy)aniline

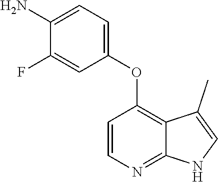 [SMILES: Cc1c[nH]c2nccc(Oc3ccc(N)c(F)c3)c12]

The title compound was prepared by following a similar procedure described for Intermediate Ie-D1, starting from tert-butyl (2-fluoro-4-((3-methyl-1-((2-(trimethylsilyl)ethoxy)methyl)-1H-pyrrolo[2,3-b]pyridin-4-yl)oxy)phenyl)carbamate (Ie-C6, 0.30 g, 0.60 mmol), TFA (5 mL), and K₂CO₃ (0.25 g, 1.80 mmol), and was obtained as a brown gum (0.20 g) which was used without further purification. LCMS: 258.1 [M+H].

### Intermediate IE-D6

### 4-((3-chloro-1H-pyrrolo[2,3-b]pyridin-4-yl)oxy)-2-methylaniline

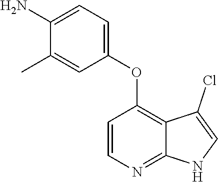 [SMILES: Cc1cc(Oc2ccnc3[nH]cc(Cl)c23)ccc1N]

HC in dioxane (4M, 0.238 mL, 0.952 mmol) was added dropwise to a solution of tert-butyl (4-((3-chloro-1-((2-(dimethylsilyl)ethoxy)methyl)-1H-pyrrolo[2,3-b]pyridin-4-yl)oxy)-2-methylphenyl)carbamate (Ie-C7, 0.480 g, 0.952 mmol) in DCM (5 mL) at 0° C. and the resulting mixture was stirred at 25° C. for 3 h. Following completion of the reaction (as indicated by LCMS), the reaction mixture was concentrated under reduced pressure to yield crude (4-(4-amino-3-methylphenoxy)-3-chloro-1H-pyrrolo[2,3-b]pyridin-1-yl)methanol (0.40 g, 1.317 mmol) which was dissolved in MeOH (10 mL) and water (3.00 mL). K₂CO₃ (0.546 g, 3.950 mmol) was then added and the resulting mixture was stirred at 25° C. for 3 h. Following completion of the reaction (as indicated by LCMS), the reaction mixture was concentrated under reduced pressure to give crude material which was diluted with EtOAc (20 mL), washed with water (5 mL×2), dried over Na₂SO₄, filtered, and concentrated under reduced pressure to afford the title compound (0.275 g) which was used without further purification. LCMS: 274.0 [M+H].

### Intermediate IE-D7

### 4-((2,3-dichloro-1-((2-(trimethylsilyl)ethoxy)methyl)-1H-pyrrolo[2,3-b]pyridin-4-yl)oxy)-2-methyl aniline

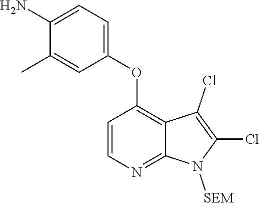 [SMILES: Cc1cc(Oc2ccnc3c2c(Cl)c(Cl)n3COCC[Si](C)(C)C)ccc1N]

HCl in dioxane (4M, 0.149 mL, 0.595 mmol) was added dropwise to a stirred solution of tert-butyl (4-((3-chloro-1-((2-(trimethylsilyl)ethoxy)methyl)-1H-pyrrolo[2,3-b]pyridin-4-yl)oxy)-2-methylphenyl)carbamate (Ie-C7, 0.30 g, 0.595 mmol) in DCM (5 mL) at 0° C. and the resulting mixture was stirred at 25° C. for 3 h. Following completion of the reaction (as indicated by LCMS), the reaction mixture was concentrated under reduced pressure to afford the title compound (0.22 g) as an HCl salt which was used without further purification. LCMS: 438.1 [M+H].

AMINE INTERMEDIATES IE-E

| | | | |
|---|---|---|---|
| Intermediate | Ie-El | Ie-E2 | Ie-E3 |
| Structure | 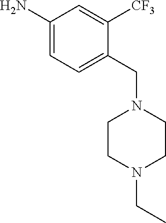 [SMILES: CCN1CCN(Cc2ccc(N)cc2C(F)(F)F)CC1] | 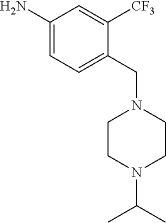 [SMILES: CC(C)N1CCN(Cc2ccc(N)cc2C(F)(F)F)CC1] | 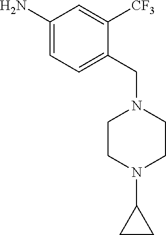 |

| | | | |
|---|---|---|---|
| Intermediate | Ie-E4 | Ie-E5 | Ie-E6 |
| Structure | 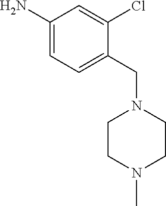 [SMILES: CN1CCN(Cc2ccc(N)cc2Cl)CC1] | 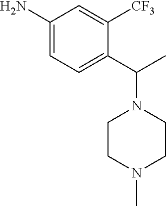 [SMILES: CC(c1ccc(N)cc1C(F)(F)F)N1CCN(C)CC1] | 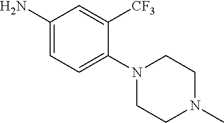 |
| Intermediate | Ie-E7 | Ie-E8 | Ie-E9 |
| Structure | 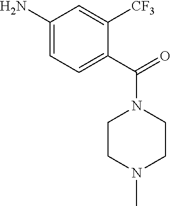 [SMILES: CN1CCN(C(=O)c2ccc(N)cc2C(F)(F)F)CC1] | 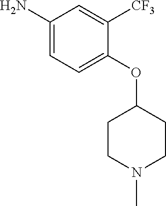 [SMILES: CN1CCC(Oc2ccc(N)cc2C(F)(F)F)CC1] | 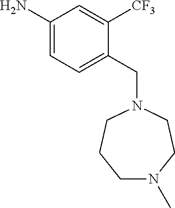 |
| Intermediate | Ie-E10 | Ie-E11 | Ie-E12 |
| Structure | 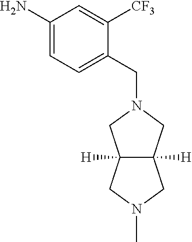 [SMILES: CN1C[C@@H]2CN(Cc3ccc(N)cc3C(F)(F)F)C[C@@H]2C1] | 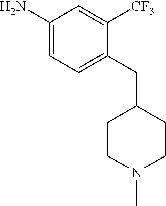 [SMILES: CN1CCC(Cc2ccc(N)cc2C(F)(F)F)CC1] | 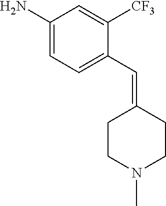 |
| Intermediate | Ie-E13 | Ie-E14 | Ie-E15 |
| Structure | 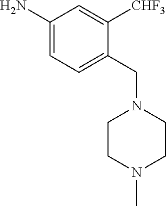 | 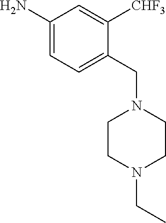 | 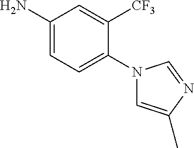 |
| Intermediate | Ie-E16 | Ie-E17 | Ie-E18 |
| Structure | 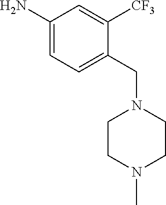 [SMILES: CN1CCN(Cc2ccc(N)cc2C(F)(F)F)CC1] | 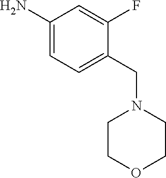 [SMILES: Nc1ccc(CN2CCOCC2)c(F)c1] | 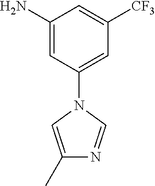 |
| Intermediate | Ie-E19 | Ie-E20 | Ie-E21 |
| Structure | 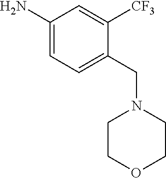 [SMILES: Nc1ccc(CN2CCOCC2)c(C(F)(F)F)c1] | 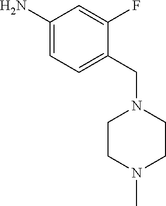 [SMILES: CN1CCN(Cc2ccc(N)cc2F)CC1] | 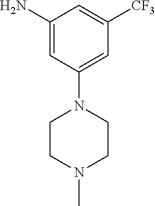 |
| Intermediate | Ie-E22 | Ie-E23 | Ie-E24 |
| Structure | 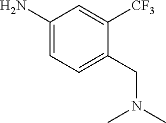 | 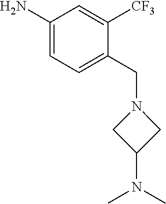 [SMILES: CN(C)C1CN(Cc2ccc(N)cc2C(F)(F)F)C1] | 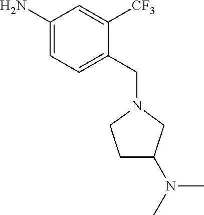 |

Intermediates Ie-E1, Ie-E6, Ie-E7, Ie-E16, and Ie-E18 are commercially available.

Intermediate Ie-E2 was prepared as reported in PCT Pub. No. WO 2018/215668.

Intermediate Ie-E3 was prepared as reported in PCT Pub. No. WO 2014/072220.

Intermediate Ie-E4 was prepared as reported in J. Med. Chem. 2015, 58(9), 39577-3974

Intermediate Ie-E5 was prepared as reported in PCT Pub. No. WO 2018/102751.

Intermediate Ie-E8 was prepared as reported in PCT Pub. No. WO 2019/200120.

Intermediate Ie-E9 was prepared as reported in PCT Pub. No. WO 2017/222285.

Intermediate Ie-E10 was prepared as reported in J. Med. Chem. 2012, 55(22), 10033-10046

Intermediates Ie-E11 and Ie-E12 were prepared as reported in PCT Pub. No. WO 2008/046802.

Intermediate Ie-E15 was prepared as reported in PCT Pub. No. WO 2016/029776.

Intermediate Ie-E17 was prepared as reported in PCT Pub. No. WO 2014/012360.

Intermediate Ie-E19 was prepared as reported in PCT Pub. No. WO 2018/215668.

Intermediate Ie-E20 was prepared as reported in PCT Pub No. WO 2020/135507.

Intermediate Ie-E21 was prepared as reported in J. Med. Chem. 2009, 52(15), 4743-4756.

Intermediate Ie-E22 was prepared as reported in ACS Med. Chem. Let. 2016, 7(6), 595-600.

Intermediate Ie-E23 was prepared as reported in PCT Pub No. WO 2020/098710.

Intermediate Ie-E24 was prepared as reported in PCT Pub. No. WO 2020/206583.

### Intermediate IE-E13

### 3-(difluoromethyl)-4-((4-methylpiperazin-1-yl)methyl)analine

### Step 1: Synthesis of 1-(2-(difluorodimethyl)-4-nitrobenzyl)-4-methylpiperazine

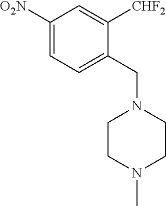 [SMILES: CN1CCN(Cc2ccc([N+](=O)[O-])cc2C(F)F)CC1]

1-Methylpiperazine (0.167 mL, 1.504 mmol) and triethylamine (0.204 mL, 1.504 mmol) were added to a solution of 1-(bromomethyl)-2-(difluoromethyl)-4-nitrobenzene (prepared as per PCT Publication No WO 2019/200120 A1, 0.40 g, 1.504 mmol) in DCM (3 mL) and the resulting mixture was stirred at 25° C. for 12 h. Following completion of the reaction (as indicated by TLC), the reaction mixture was concentrated under reduced pressure, giving crude material which was purified by Isolera (silica gel 230-400 mesh, eluting with using 5% MeOH in DCM), affording the title compound as a yellow solid (0.09 g, 21% yield). LCMS: 286.1 [M+H].

### Step 2: Synthesis of 3-(difluoromethyl)-4-((4-methylpiperazin-1-yl)methyl)aniline

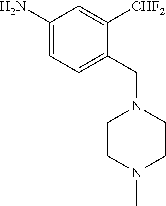 [SMILES: CN1CCN(Cc2ccc(N)cc2C(F)F)CC1]

Iron powder (0.157 g, 2.80 mmol) and NH₄Cl (0.150 g, 2.80 mmol) were added to a solution of 1-(2-(difluoromethyl)-4-nitrobenzyl)-4-methylpiperazine (0.080 g, 0.280 mmol) in ethanol (2 mL) and water (2 mL) and the resulting suspension was stirred at 80° C. for 1 h. Following completion of the reaction (as indicated by TLC), the reaction mixture was diluted with DCM (10 mL) and filtered through a pad of of diatomaceous earth which was then washed with DCM (5 mL). The combined filtrates were concentrated under reduced pressure, affording the title compound as an off-white solid (0.07 g) which was used without further purification. LCMS: 256.2 [M+H].

### Intermediate IE-E14

### 3-(difluoromethyl)-4-((4-ethylpiperazin-1-yl)methyl)aniline

### Step 1: Syntheses of 1-(2-(difluoromethyl)-4-nitrophenyl)-4-ethylpiperazine

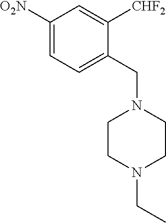 [SMILES: CCN1CCN(Cc2ccc([N+](=O)[O-])cc2C(F)F)CC1]

The title compound was prepared by following a similar procedure described for Intermediate Ie-E13 (step 1), starting from 1-(bromomethyl)-2-(difluoromethyl)-4-nitrobenzene (0.40 g, 1.504 mmol) and 1-ethylpiperazine (0.191 mL, 1.504 mmol), and was obtained as a yellow solid (0.10 g, 22% yield). LCMS: 300.1 [M+H].

### Step 2: Synthesis of 3-(difluoromethyl)-4-((4-ethylpiperazin-1-yl)methyl)aniline

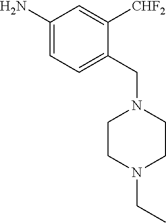 [SMILES: CCN1CCN(Cc2ccc(N)cc2C(F)F)CC1]

The title compound was prepared by following a similar procedure described for Intermediate Ie-E13 (step 2), starting from 1-(2-(difluoromethyl)-4-nitrobenzyl)-4-ethylpiperazine (0.100 g, 0.334 mmol), iron powder (0.187 g, 3.34 mmol), and NH₄Cl (0.179 g, 3.34 mmol), and was obtained as as pale yellow solid (0.093 g) which was used without further purification. LCMS: 270.1 [M+H].

### Carbamate Intermediates IE-F

### General Procedure for the Synthesis of Carbamate Intermediates Ie-F

Pyridine (1.2 eq) and phenyl chloroformate (1.5 eq) were added to a solution of amine (1.0 eq) in THF (10 vol) at 0° C. The reaction mixture was allowed to warm to 25° C. and was stirred for 16 hi. Following completion of the reaction (as indicated by TLC):

(i) the precipitated solid was filtered, washed with THF, and dried to afford the desired carbamate OR

(ii) the reaction mixture was diluted with EtOAc (10 mL) and washed with brine (5 mL). The organic layer was dried over Na₂SO₄, filtered, and concentrated under reduced pressure to yield crude material which was purified by flash chromatography (silica gel 230-400 mesh, eluting with 10 to 20% EtOAc in petroleum ether), giving the desired carbamate.

The following carbamates were prepared using the above general procedure:

| | Carbamate structure | Amine structure | |
|---|---|---|---|
| Intermediate | (product) | (starting material) | LCMS |
| Ie-F1 | 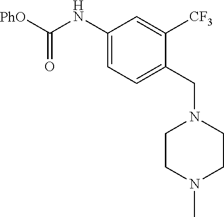 [SMILES: CN1CCN(Cc2ccc(NC(=O)Oc3ccccc3)cc2C(F)(F)F)CC1] | 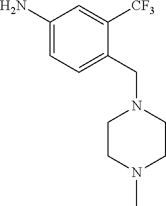 | 394.2 [M + H] |
| | | E16 | |
| Ie-F2 | 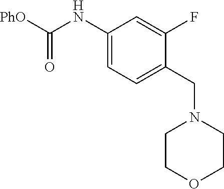 [SMILES: O=C(Nc1ccc(CN2CCOCC2)c(F)c1)Oc1ccccc1] | 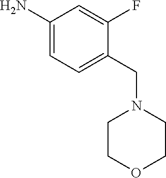 [SMILES: Nc1ccc(CN2CCOCC2)c(F)c1] | 331.1 [M + H] |
| | | E17 | |
| Ie-F3 | 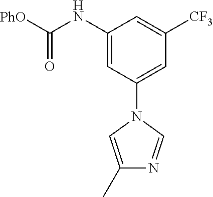 [SMILES: Cc1cn(-c2cc(NC(=O)Oc3ccccc3)cc(C(F)(F)F)c2)cn1] | 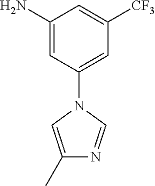 | 362.0 [M + H] |
| | | E18 | |
| Ie-F4 | 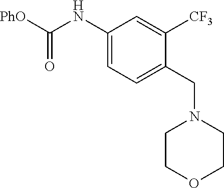 [SMILES: O=C(Nc1ccc(CN2CCOCC2)c(C(F)(F)F)c1)Oc1ccccc1] | 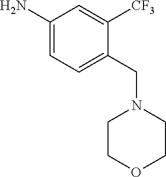 [SMILES: Nc1ccc(CN2CCOCC2)c(C(F)(F)F)c1] | 381.1 [M + H] |
| | | E19 | |
| Ie-F5 | 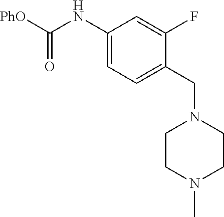 [SMILES: CN1CCN(Cc2ccc(NC(=O)Oc3ccccc3)cc2F)CC1] | 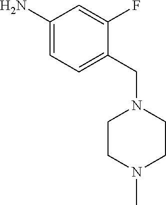 | 344.2 [M + H] |
| | | E20 | |
| Ie-F6 | 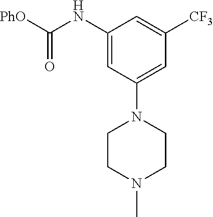 [SMILES: CN1CCN(c2cc(NC(=O)Oc3ccccc3)cc(C(F)(F)F)c2)CC1] | 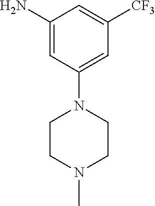 | 380.1 [M + H] |
| | | E21 | |
| Ie-F7 | 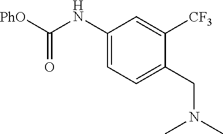 [SMILES: CN(C)Cc1ccc(NC(=O)Oc2ccccc2)cc1C(F)(F)F] | 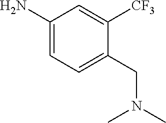 | 339.1 [M + H] |
| | | E22 | |
| Ie-F8 | 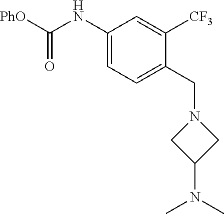 [SMILES: CN(C)C1CN(Cc2ccc(NC(=O)Oc3ccccc3)cc2C(F)(F)F)C1] | 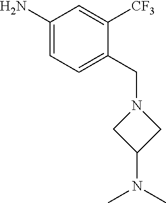 | 394.1 [M + H] |
| | | E24 | |
| Ie-F9 | 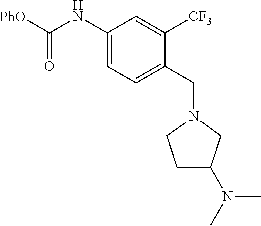 [SMILES: CN(C)C1CCN(Cc2ccc(NC(=O)Oc3ccccc3)cc2C(F)(F)F)C1] | 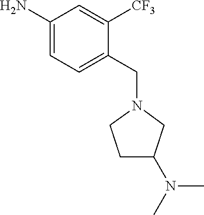 | 408.1 [M + H] |
| | | E24 | |
| Ie-F10 | 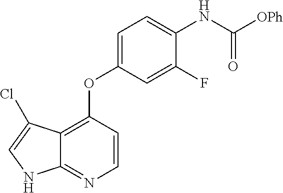 [SMILES: O=C(Nc1ccc(Oc2ccnc3[nH]cc(Cl)c23)cc1F)Oc1ccccc1] | 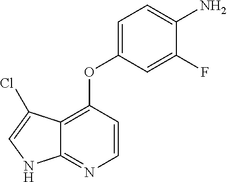 [SMILES: Nc1ccc(Oc2ccnc3[nH]cc(Cl)c23)cc1F] | 398.1 [M + H] |
| | | D1 | |
| Ie-F11 | 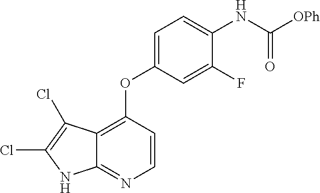 [SMILES: O=C(Nc1ccc(Oc2ccnc3[nH]c(Cl)c(Cl)c23)cc1F)Oc1ccccc1] | 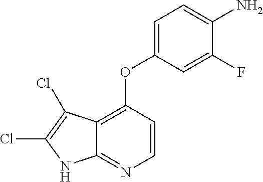 [SMILES: Nc1ccc(Oc2ccnc3[nH]c(Cl)c(Cl)c23)cc1F] | 432.0 [M + H] |
| | | D2 | |
| Ie-F12 | 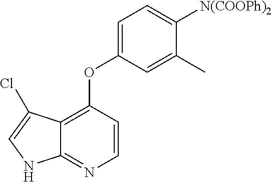 | 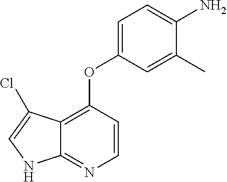 | 514.1 [M + H] |
| | | D6 | |

### Preparation of Examples

General Urea Formation Procedure for the Synthesis of Examples Ie-1 Through Ie-30

Method Ie-A Triethylamine (2.0 eq) was added to a mixture of carbamate Intermediate Ie-C (1.0 eq.) and amine Intermediate Ie-D (1.0 eq.) in THF (S mL) and the resulting mixture was stirred at 60° C. for 12 h in a sealed tube. Following completion of the reaction (as indicated by LCMS), the reaction mixture was concentrated under reduced pressure to give crude material which was purified by reverse phase preparative HPLC, affording the title product.

Method Ie-B DMAP (0.05 eq.) and DIPEA (1.5 eq.) were added to a solution of carbamate Intermediate Ie-C (1.0 eq.) and amine Intermediate Ie-D (1.0 eq.) in THE (10 vol) and the resulting mixture was stirred at 60° C. for 12 h in a sealed tube. Following completion of the reaction (as indicated by LCMS), the reaction mixture was concentrated under reduced pressure to yield crude material which was purified by reverse phase preparative HPLC, affording the title product.

### Example IE-1

### 1-(4-((3-chloro-1H-pyrrolo[2,3-b]pyridin-4-yl)oxy)-2-fluorophenyl)-3-(4-((4-methylpiperazin-1-yl)methyl)-3-(trifluoromethyl)phenyl)urea

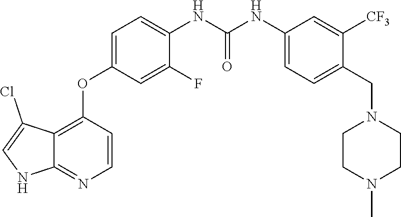 [SMILES: CN1CCN(Cc2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.100 g, 0.360 mmol) and phenyl (4-((4-methylpiperazin-1-yl)methyl)-3-(trifluoromethyl)phenyl)carbamate (Ie-F1, 0.142 g, 0.360 mmol), and was obtained as an off-white solid (52 mg, 25% yield). ¹H NMR (400 MHz, DMSO-d₆) δ=12.09 (bs, 1H), 9.43 (bs, 1H), 8.68 (bs, 1H), 8.12-8.16 (m, 2H), 7.99 (d, J=2.0 Hz, 1H), 7.59-7.65 (m, 3H), 7.26-7.29 (m, 1H), 7.01-7.03 (m, 1H), 6.42 (d, J=5.6 Hz, 1H), 3.58 (s, 2H), 2.43-2.79 (m, 1H). LCMS: 577.1 [M+H].

### Example IE-2

### 1-(4-((3-chloro-1H-pyrrolo[2,3-b]pyridin-4-yl)oxy)-2-fluorophenyl)-3-(3-fluoro-4-(morpholinomethyl)phenyl)urea

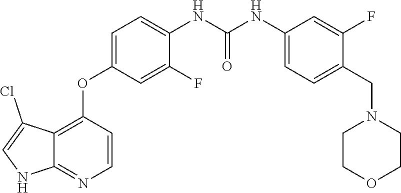 [SMILES: O=C(Nc1ccc(CN2CCOCC2)c(F)c1)Nc1ccc(Oc2ccnc3[nH]cc(Cl)c23)cc1F]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.075 g, 0.37 mmol) and phenyl (3-fluoro-4-(morpholinomethyl)phenyl)carbamate (Ie-F2, 0.089 g, 0.27 mmol), and was obtained as an off-white solid (10 mg, 7% yield). ¹H NMR (400 MHz, DMSO-d₆) δ=12.08 (bs, 1H), 9.05 (bs, 1H), 9.00 (bs, 1H), 8.19-8.24 (m, 1H), 8.08-8.14 (m, 2H), 7.59 (d, J=2.8 Hz, 1H), 7.01-7.29 (m, 4H), 6.42 (d, J=5.6 Hz, 1H), 3.58 (bs, 4H), 3.43 (bs, 2H), 2.33-2.35 (m, 4H). LCMS: 514.2 [M+H].

### Example IE-3

### 1-(4-((3-Chloro-1H-pyrrolo[2,3-b]pyridin-4-yl)oxy)-2-fluorophenyl)-3-(3-(4-methyl-1H-1-imidazol-1-yl)-5-(TRIFLUOROMETHYL)phenyl)urea

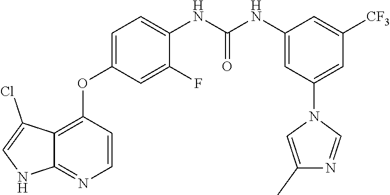 [SMILES: Cc1cn(-c2cc(NC(=O)Nc3ccc(Oc4ccnc5[nH]cc(Cl)c45)cc3F)cc(C(F)(F)F)c2)cn1]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.070 g, 0.252 mmol) and phenyl (3-(4-methyl-1H-imidazol-1-yl)-5-(trifluoromethyl)phenyl)carbamate (Ie-F3, 0.091 g, 0.252 mmol), and was obtained as an off-white solid (20 mg, 15% yield). ¹H NMR (400 MHz, DMSO-d₆) δ=12.09 (bs, 1H), 10.50 (bs, 1H), 9.70 (bs, 1H), 8.20 (d, J=1.2 Hz, 1H), 8.15 (d, J=5.6 Hz, 1H), 7.98-8.05 (m, 1H), 7.91 (bs, 2H), 7.60 (bs, 1H), 7.56 (bs, 1H), 7.48 (s, 1H), 7.23-7.27 (m, 1H), 7.00-7.03 (m, 1H), 6.43 (d, J=5.2 Hz, 1H), 2.18 (s, 3H). LCMS: 545.1 [M+H].

### Example IE-4

### 1-(4-((3-Chloro-1H-pyrrolo[2,3-b]pyridin-4-yl)oxy)-2-fluorophenyl)-3-(4-(morpholinomethyl)-3-(trifluoromethyl)phenyl)UR EA

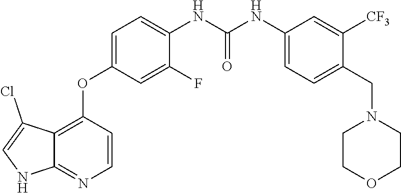 [SMILES: O=C(Nc1ccc(CN2CCOCC2)c(C(F)(F)F)c1)Nc1ccc(Oc2ccnc3[nH]cc(Cl)c23)cc1F]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.070 g, 0.252 mmol) and phenyl (4-(morpholinomethyl)-3-(trifluoromethyl)phenyl)carbamate (Ie-F4, 0.096 g, 0.252 mmol), and was obtained as an off-white solid (17 mg, 12% yield). ¹H NMR (400 MHz, DMSO-d₆) δ=12.09 (bs, 1H), 9.36 (bs, 1H), 8.64 (bs, 1H), 8.13-8.17 (m, 2H), 8.01 (d, J=2.0 Hz, 1H), 7.68 (d, J=8.8 Hz, 1H), 7.55-7.60 (m, 2H), 7.26-7.29 (m, 1H), 7.01-7.03 (m, 1H), 6.42 (d, J=5.6 Hz, 1H), 3.58-3.60 (m, 4H), 3.55 (s, 2H), 2.33-2.34 (m, 4H). LCMS: 564.1 [M+H].

### Example IE-5

### 1-(4-((3-chloro-1H-pyrrolo[2,3-b]pyridin-4-yl)oxy)-2-fluorophenyl)-3-(3-fluoro-4-((4-methylpiperazin-1-yl)methyl)phenyl)urea

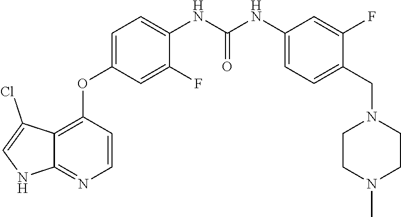 [SMILES: CN1CCN(Cc2ccc(NC(=O)Nc3ccc(Oc4ccnc5[nH]cc(Cl)c45)cc3F)cc2F)CC1]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.080 g, 0.288 mmol) and phenyl (3-fluoro-4-((4-methylpiperazin-1-yl)methyl)phenyl)carbamate (Ie-F5, 0.099 g, 0.288 mmol), and was obtained as an off-white solid (9 mg, 6% yield). ¹H NMR (400 MHz, DMSO-d₆) δ=9.47 (bs, 1H), 8.84 (bs, 1H), 8.11-8.15 (m, 2H), 7.59 (s, 1H), 7.46-7.50 (m, 1H), 7.24-7.29 (m, 2H), 7.08-7.11 (m, 1H), 7.00-7.03 (m, 1H), 6.42 (d, J=5.6 Hz, 1H), 3.44 (s, 2H), 2.30-2.34 (m, 81), 2.14 (s, 3H). LCMS: 527.2 [M+H].

### Example IE-6

### 1-(4-((3-chloro-1H-pyrrolo[2,3-b]pyridin-4-yl)oxy)-2-fluorophenyl)-3-(3-(4-methylpiperazin-1-yl)-5-(trifluoromethyl)phenyl)urea

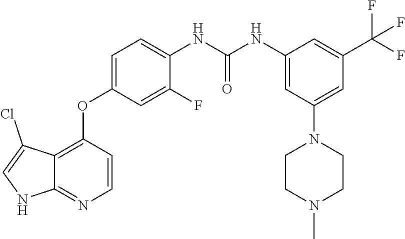 [SMILES: CN1CCN(c2cc(NC(=O)Nc3ccc(Oc4ccnc5[nH]cc(Cl)c45)cc3F)cc(C(F)(F)F)c2)CC1]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.075 g, 0.270 mmol) and phenyl (3-(4-methylpiperazin-1-yl)-5-(trifluoromethyl)phenyl)carbamate (Ie-F6, 0.102 g, 0.270 mmol), and was obtained as an off-white solid (4 mg, 2% yield). ¹H NMR (400 MHz, CD₃OD) δ=8.11-8.17 (m, 2H), 7.37 (bs, 1H), 7.35 (bs, 1H), 7.24 (bs, 1H), 7.07-7.11 (m, 1H), 7.00-7.03 (m, 1H), 6.89 (bs, 1H), 6.49 (d, J=5.6 Hz, 1H), 3.30-3.31 (m, 4H), 2.66-2.69 (m, 4H), 2.40 (s, 3H). LCMS: 563.1 [M+H].

### Example IE-7

### 1-(4-((3-chloro-1H-pyrrolo[2,3-b]pyridin-4-yl)oxy)-2-fluorophenyl)-3-(4-((dimethylamino)methyl)-3-(trifluoromethyl)phenyl)urea

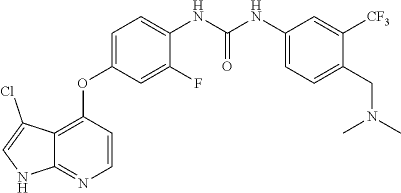 [SMILES: CN(C)Cc1ccc(NC(=O)Nc2ccc(Oc3ccnc4[nH]cc(Cl)c34)cc2F)cc1C(F)(F)F]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.139 g, 0.501 mmol) and phenyl (4-((dimethylamino)methyl)-3-(trifluoromethyl)phenyl)carbamate (Ie-F7, 0.169 g, 0.501 mmol), and was obtained as an off-white solid (9 mg, 3% yield). ¹H NMR (400 MHz, CD₃OD) δ=6 8.20-8.24 (m, 2H), 8.14 (d, J=2.4 Hz, 1H), 7.85-7.88 (m, 1H), 7.70-7.72 (m, 1H), 7.50 (s, 1H), 7.19-7.23 (m, 1H), 7.09-7.13 (m, 1H), 6.65 (d, J=6.4 Hz, 1H), 4.49 (s, 2H), 2.95 (s, 6H). LCMS: 522.1 [M+H].

### Example IE-8

### 1-(4-((3-chloro-1H-pyrrolo[2,3-b]pyridin-4-yl)oxy)-2-fluorophenyl)-3-(4-((3-(dimethylamino)azetidin-1-yl)methyl)-3-(trifluoromethyl)phenyl)urea

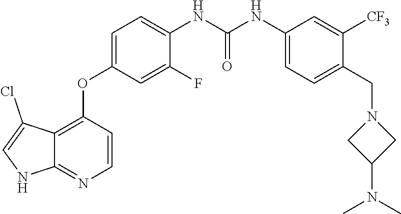 [SMILES: CN(C)C1CN(Cc2ccc(NC(=O)Nc3ccc(Oc4ccnc5[nH]cc(Cl)c45)cc3F)cc2C(F)(F)F)C1]

The title compound was prepared following the general procedure for urea formation (Method Ie-A), starting from 4-((3-chloro-1H-pyrrolo[2,3-b]pyridin-4-yl)oxy)-2-fluoroaniline (Ie-D1, 0.05 g, 0.180 mmol) and phenyl (4-((3-(dimethylamino)azetidin-1-yl))methyl)-3-(trifluoromethyl)phenyl)carbamate (Ie-F8, 0.071 g, 0.180 mmol), and was obtained as an off-white solid (11 mg, 19% yield). ¹H NMR (400 MHz, CD₃OD) δ=8.09-8.14 (m, 2H), 7.96 (d, J=2.0 Hz, 1H), 7.67-7.70 (m, 1H), 7.59-7.62 (m, 1H), 7.34 (s, 1H), 7.06-7.10 (m, 1H), 6.99-7.02 (m, 1H), 6.49 (d, J=5.6 Hz, 1H), 3.96 (s, 2H), 3.74-3.78 (m, 2H), 3.55-3.58 (m, 1H), 3.40-3.44 (m, 2H), 2.55 (s, 6H). LCMS: 577.1 [M+H].

### Example IE-9

### 1-(4-((3-chloro-1H-pyrrolo[2,3-b]pyridin-4-yl)oxy)-2-fluorophenyl)-3-(4-((4-ethylpiperazin-1-yl)methyl)-3-(trifluoromethyl)phenyl)urea

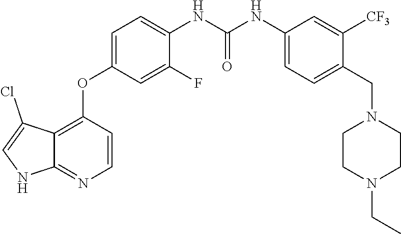 [SMILES: CCN1CCN(Cc2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.020 g, 0.05 mmol) and 4-((4-ethylpiperazin-1-yl)methyl)-3-(trifluoromethyl)aniline (Ie-E1, 0.014 g, 0.05 mmol), and was obtained as an off-white solid (0.6 mg, 2% yield). ¹H NMR (400 MHz, CD₃OD) δ=8.10-8.13 (m, 2H), 7.90 (s, 1H), 7.71 (bs, 2H), 7.35 (s, 1H), 7.08-7.11 (m, 1H), 7.01-7.03 (m, 1H), 6.50 (d, J=5.6 Hz, 1H), 3.76 (s, 2H), 3.15-3.51 (m, 8H), 2.48 (bs, 2H), 1.37 (t, J=7.2 Hz, 3H). LCMS: 591.2 [M+H].

### Example IE-10

### 1-(4-((3-chloro-1H-pyrrolo[2,3-b]pyridin-4-yl)oxy)-2-fluorophenyl)-3-(4-((4-isopropylpiperazin-1-yl)methyl)-3-(trifluoromethyl)phenyl)urea

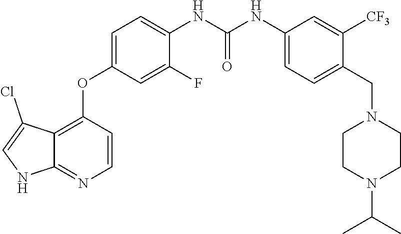 [SMILES: CC(C)N1CCN(Cc2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.100 g, 0.25 mmol) and 4-((4-isopropylpiperazin-1-yl)methyl)-3-(trifluoromethyl)aniline (Ie-E2, 0.076 g, 0.25 mmol), and was obtained as an off-white solid (6 mg, 4% yield). ¹H NMR (400 MHz, CD₃OD) δ=8.11-8.13 (m, 2H), 7.91 (s, 1H), 7.66-7.73 (m, 2H), 7.35 (s, 1H), 7.07-7.11 (m, 1H), 7.00-7.03 (m, 1H), 6.49 (d, J=5.6 Hz, 1H), 3.70 (s, 2H), 3.10-3.18 (m, 1H), 3.01 (bs, 4H), 2.69 (bs, 4H), 1.28-1.31 (m, 6H). LCMS: 605.2 [M+H].

### Example IE-11

### 1-(4-((3-chloro-1H-pyrrolo[2,3-b]pyridin-4-yl)oxy)-2-fluorophenyl)-3-(4-((4-cyclopropylpiperazin-1-yl)methyl)-3-(trifluoromethyl)phenyl)urea

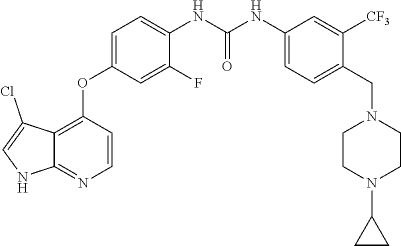 [SMILES: O=C(Nc1ccc(CN2CCN(C3CC3)CC2)c(C(F)(F)F)c1)Nc1ccc(Oc2ccnc3[nH]cc(Cl)c23)cc1F]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.100 g, 0.25 mmol) and 4-((4-cyclopropylpiperazin-1-yl)methyl)-3-(trifluoromethyl)aniline (Ie-E3, 0.075 g, 0.25 mmol), and was obtained as an off-white solid (3 mg, 2% yield). ¹H NMR (400 MHz, CD₃OD) δ=8.11-8.16 (m, 2H), 7.94 (bs, 1H), 7.73 (bs, 2H), 7.37 (bs, 1H), 7.09-7.13 (m, 1H), 7.02-7.05 (m, 1H), 6.52 (d, J=5.6 Hz, 1H), 3.69 (s, 2H), 2.77 (bs, 4H), 2.54 (bs, 4H), 1.77-1.80 (m, 1H), 0.47-0.55 (m, 4H). LCMS: 603.2 [M+H].

### Example IE-12

### 1-(4-((3-chloro-1H-pyrrolo[2,3-b]pyridin-4-yl)oxy)-2-fluorophenyl)-3-(3-chloro-4-((4-methylpiperazin-1-yl)methyl)phenyl)urea

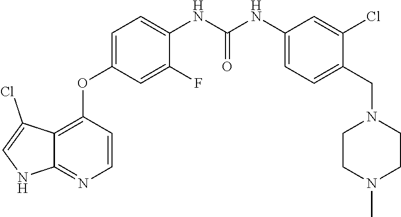 [SMILES: CN1CCN(Cc2ccc(NC(=O)Nc3ccc(Oc4ccnc5[nH]cc(Cl)c45)cc3F)cc2Cl)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.070 g, 0.176 mmol) and 3-chloro-4-((4-methylpiperazin-1-yl)methyl)aniline (Ie-E4, 0.042 g, 0.176 mmol), and was obtained as an off-white solid (6 mg, 6% yield). ¹H NMR (400 MHz, CD₃OD) δ=8.13-8.17 (m, 2H), 7.71 (d, J=2.0 Hz, 1H), 7.36-7.43 (m, 3H), 7.02-7.13 (m, 2H), 6.55 (d, J=6.0 Hz, 1H), 3.76 (s, 2H), 2.90 (s, 3H), 2.65 (bs, 81). LCMS: 543.2 [M+H].

### Example IE-13

### 1-(4-((3-chloro-1H-pyrrolo[2,3-b]pyridin-4-yl)oxy)-2-fluorophenyl)-3-(4-(1-(4-methylpiperazin-1-yl)ethyl)-3-(trifluoromethyl)phenyl)urea

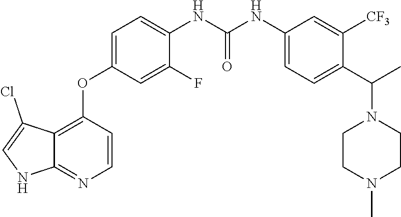 [SMILES: CC(c1ccc(NC(=O)Nc2ccc(Oc3ccnc4[nH]cc(Cl)c34)cc2F)cc1C(F)(F)F)N1CCN(C)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.06 g, 0.15 mmol) and 4-(1-(4-methylpiperazin-1-yl)ethyl)-3-(trifluoromethyl)aniline (Ie-E5, 0.043 g, 0.15 mmol), and was obtained as an off-white solid (5 mg, 6% yield). ¹H NMR (400 MHz, CD₃OD) δ=8.10-8.14 (m, 2H), 7.88 (d, J=2.0 Hz, 1H), 7.78-7.80 (m, 1H), 7.66-7.68 (m, 1H), 7.34 (s, 1H), 6.95-7.09 (m, 2H), 6.48 (d, J=5.6 Hz, 1H), 3.67-3.69 (m, 1H), 2.83 (bs, 4H), 2.54 (s, 3H), 2.47 (bs, 4H), 1.29-1.37 (m, 3H). LCMS: 591.2 [M+H].

### Example IE-14

### 1-(4-((3-chloro-1H-pyrrolo[2,3-b]pyridin-4-yl)oxy)-2-fluorophenyl)-3-(4-(4-methylpiperazin-1-yl)-3-(trifluoromethyl)phenyl)urea

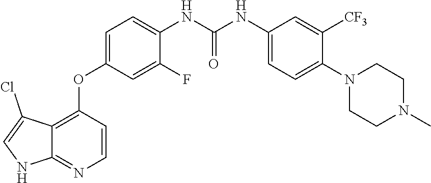 [SMILES: CN1CCN(c2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.100 g, 0.251 mmol) and 4-(4-methylpiperazin-1-yl)-3-(trifluoromethyl)aniline (Ie-E6, 0.072 g, 0.277 mmol), and was obtained as an off-white solid (83 mg, 57% yield). ¹H NMR (400 MHz, CD₃OD) δ=8.14-8.19 (m, 2H), 7.91 (d, J=2.4 Hz, 1H), 7.74-7.76 (m, 1H), 7.52-7.54 (m, 1H), 7.41 (s, 1H), 7.03-7.16 (m, 2H), 6.56 (d, J=6.0 Hz, 1H), 3.59-3.61 (m, 2H), 3.19-3.30 (m, 61), 3.01 (s, 3H). LCMS: 563.2 [M+H].

### Example IE-15

### 1-(4-((3-chloro-1H-pyrrolo[2,3-b]pyridin-4-yl)oxy)-2-fluorophenyl)-3-(4-(4-methylpiperazine-1-carbonyl)-3-(trifluoromethyl)phenyl)urea

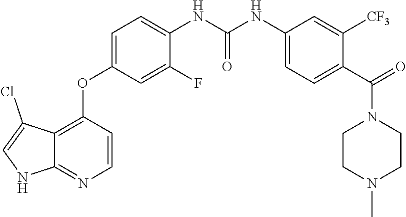 [SMILES: CN1CCN(C(=O)c2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.100 g, 0.251 mmol) and (4-amino-2-(trifluoromethyl)phenyl)(4-methylpiperazin-1-yl)methanone (Ie-E7, 0.079 g, 0.277 mmol), and was obtained as an off-white solid (16 mg, 10% yield). ¹H NMR (400 MHz, CD₃OD) δ=8.15-8.19 (m, 2H), 8.06 (bs, 1H), 7.81-7.83 (m, 1H), 7.47-7.49 (m, 1H), 7.40 (s, 1H), 7.12-7.16 (m, 1H), 7.04-7.07 (m, 1H), 6.55 (d, J=5.6 Hz, 1H), 3.54-3.60 (m, 8H), 3.00 (s, 3H). LCMS: 591.2 [M+H].

### Example IE-16

### 1-(4-((3-chloro-1H-pyrrolo[2,3-b]pyridin-4-yl)oxy)-2-fluorophenyl)-3-(4-((1-methylpiperidin-4-yl)oxy)-3-(trifluoromethyl)phenyl)urea

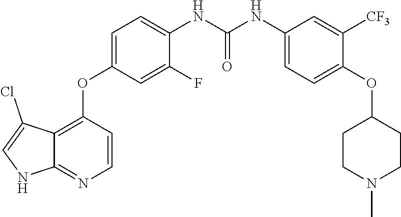 [SMILES: CN1CCC(Oc2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.090 g, 0.226 mmol) and 4-((1-methylpiperidin-4-yl)oxy)-3-(trifluoromethyl)aniline (Ie-E8, 0.062 g, 0.226 mmol), and was obtained as an off-white solid (15 mg, 11% yield). ¹H NMR (400 MHz, CD₃OD) δ=8.09-8.14 (m, 2H), 7.79 (d, J=2.8 Hz, 1H), 7.62-7.65 (m, 1H), 7.34 (s, 1H), 7.21-7.23 (m, 1H), 7.07-7.10 (m, 1H), 6.99-7.02 (m, 1H), 6.49 (d, J=5.6 Hz, 1H), 4.75 (bs, 1H), 3.14-3.16 (m, 2H), 2.96-3.04 (m, 2H), 2.64 (s, 3H), 2.07-2.18 (m, 4H). LCMS: 578.2 [M+H].

### Example IE-17

### 1-(4-((3-chloro-1H-pyrrolo[2,3-b]pyridin-4-yl)oxy)-2-fluorophenyl)-3-(4-((4-methyl-1,4-diazepan-1-yl)methyl)-3-(trifluoromethyl)phenyl)urea

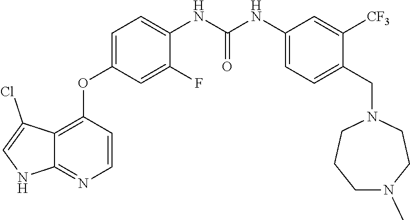 [SMILES: CN1CCCN(Cc2ccc(NC(=O)Nc3ccc(Oc4ccnc5[nH]cc(Cl)c45)cc3F)cc2C(F)(F)F)CC1]

The title compound was prepared following the general procedure for urea formation (Method B), starting from phenyl (4-((3-chloro-1H-pyrrolo[2,3-b]pyridin-4-yl)oxy)-2-fluorophenyl)carbamate (Ie-F10, 0.100 g, 0.251 mmol) and 4-((4-methyl-1,4-diazepan-1-yl)methyl)-3-(trifluoromethyl)aniline (Ie-E9, 0.072 g, 0.251 mmol), and was obtained as a pale brown solid (0.6 mg, 0.4% yield). ¹H NMR (400 MHz, CD₃OD) δ=8.15-8.20 (m, 2H), 7.91 (d, J=2.0 Hz, 1H), 7.72-7.79 (m, 2H), 7.42 (s, 1H), 7.13-7.16 (m, 1H), 7.04-7.07 (m, 1H), 6.57 (d, J=6.0 Hz, 1H), 3.92 (bs, 2H), 3.50-3.50 (m, 2H), 2.90-3.00 (m, 71), 2.12 (bs, 2H) LCMS: 591.2 [M+H].

### Example IE-18

### 1-(4-((3-chloro-1H-pyrrolo[2,3-b]pyridin-4-yl)oxy)-2-fluorophenyl)-3-(4-(((3aR,6aS)-5-methylhexahydropyrrolo[3,4-c]pyrrol-2(1H)-yl)methyl)-3-(trifluoromethyl)phenyl)urea

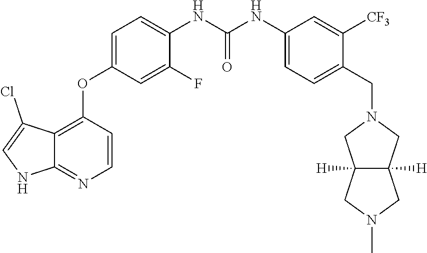 [SMILES: CN1C[C@@H]2CN(Cc3ccc(NC(=O)Nc4ccc(Oc5ccnc6[nH]cc(Cl)c56)cc4F)cc3C(F)(F)F)C[C@@H]2C1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.100 g, 0.251 mmol) and 4-(((3aR,6aS)-5-methylhexahydropyrrolo[3,4-c]pyrrol-2(1H)-yl))methyl)-3-(trifluoromethyl)aniline (Ie-E10, 0.075 g, 0.251 mmol), and was obtained as a pale brown solid (7 mg, 4% yield). ¹H NMR (400 MHz, CD₃OD) δ=8.16-8.20 (m, 2H), 8.04 (d, J=2.0 Hz, 1H), 7.74-7.78 (m, 2H), 7.43 (s, 1H), 7.14-7.18 (m, 1H), 7.05-7.09 (m, 1H), 6.59 (d, J=6.0 Hz, 1H), 4.32 (s, 2H), 3.13-3.32 (m, 10H), 2.98 (s, 3H). LCMS: 603.2 [M+H].

### Example IE-19 1-(4-((3-chloro-1H-pyrrolo[2,3-H]pyridin-4-yl)oxy)-2-fluorophenyl)-3-(4-((1-methylpiperidin-4-yl)methyl)-3-(trifluoromethyl)phenyl)urea

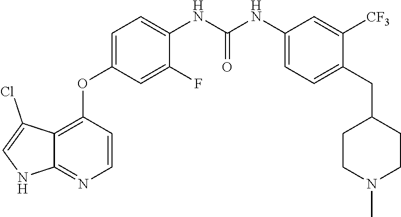 [SMILES: CN1CCC(Cc2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.060 g, 0.151 mmol) and 4-(1-methylpiperidin-4-yl)methyl)-3-(trifluoromethyl)aniline (Ie-E11, 0.045 g, 0.166 mmol), and was obtained as a brown gum (22 mg, 24% yield). ¹H NMR (400 MHz, CD₃OD) δ=8 12-8.16 (m, 2H), 7.90 (d, J=2.4 Hz, 1H), 7.64-7.67 (m, 1H), 7.37-7.41 (m, 2H), 7.09-7.13 (m, 1H), 7.01-7.04 (m, 1H), 6.53 (d, J=5.6 Hz, 1H), 3.50-3.53 (m, 2H), 2.94-3.00 (m, 2H), 2.77-2.85 (m, 5H), 1.92-1.99 (m, 3H), 1.50-1.59 (m, 2H). LCMS 576.2 [M+H].

### Example IE-20

### 1-(4-((3-chloro-1-pyrrolo[2,3-b]pyridin-4-yl)oxy)-2-fluorophenyl)-3-(4-((1-methylpiperidin-4-ylidene)methyl)-3-(trifluoromethyl)phenyl)urea

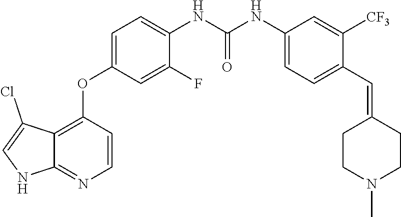 [SMILES: CN1CCC(=Cc2ccc(NC(=O)Nc3ccc(Oc4ccnc5[nH]cc(Cl)c45)cc3F)cc2C(F)(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.050 g, 0.126 mmol) and 4-((1-methylpiperidin-4-ylidene)methyl)-3-(trifluoromethyl)aniline (Ie-E12, 0.037 g, 0.138 mmol), and was obtained as a white gum (7 mg, 10% yield). ¹H NMR (400 MHz, CD₃OD) δ=8.13-8.17 (m, 2H), 7.93 (d, J=2.4 Hz, 1H), 7.70-7.72 (m, 1H), 7.38 (s, 1H), 7.28-7.30 (m, 1H), 7.10-7.13 (m, 11H), 7.02-7.05 (m, 1H), 6.69 (s, 1H), 6.53 (d, J=5.6 Hz, 1H), 3.51-3.68 (m, 2H), 2.96-3.16 (m, 5H), 2.68 (bs, 3H), 2.37-2.40 (m, 1H). LCMS: 574.2 [M+H].

### Example IE-21

### 1-(4-((3-chloro-1H-pyrrolo[2,3-b]pyridin-4-yl)oxy)-2-fluorophenyl)-3-(4-((3-(dimethylamino)pyrrolidin-1-yl)methyl)-3-(trifluoromethyl)phenyl)urea

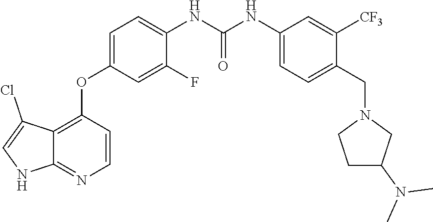 [SMILES: CN(C)C1CCN(Cc2ccc(NC(=O)Nc3ccc(Oc4ccnc5[nH]cc(Cl)c45)cc3F)cc2C(F)(F)F)C1]

The title compound was prepared following the general procedure for urea formation (Method Ie-B), starting from 4-((3-chloro-11-pyrrolo[2,3-b]pyridin-4-yl)oxy)-2-fluoroaniline (Ie-D1, 0.070 g, 0.252 mmol) and phenyl (4-((3-(dimethylamino)pyrrolidin-1-yl)methyl)-3-(trifluoromethyl)phenyl)carbamate (Ie-F9, 0.103 g, 0.252 mmol), and was obtained as a white solid (9 mg, 5.4% yield). ¹H NMR (400 MHz, DMSO-d₆) δ=12.12 (bs, 1H), 10.51 (bs, 1H), 9.71 (bs, 1H), 7.97-8.14 (m, 3H), 7.59-7.66 (m, 2H), 7.20-7.24 (m, 1H), 6.98-7.01 (m, 1H), 6.43 (d, J=5.6 Hz, 1H), 3.62-3.65 (m, 2H), 2.64-2.74 (m, 4H), 2.33-2.34 (m, 2H), 2.09 (bs, 6H), 1.84-1.88 (m, 1H), 1.60-1.65 (m, 1H), LCMS: 590.8 [M+H].

### Example IE-22

### 1-(4-((3-chloro-1H-pyrrolo[2,3-b]pyridin-4-yl)oxy)-2-fluorophenyl)-3-(3-(difluoromethyl)-4-((4-methylpiperazin-1-yl)methyl)phenyl)urea

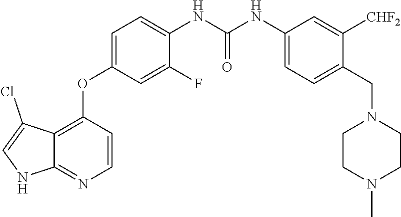 [SMILES: CN1CCN(Cc2ccc(NC(=O)Nc3ccc(Oc4ccnc5[nH]cc(Cl)c45)cc3F)cc2C(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.150 g, 0.377 mmol) and 3-(difluoromethyl)-4-((4-methylpiperazin-1-yl)methyl)aniline (Ie-E13, 0.096 g, 0.377 mmol), and was obtained as a white solid (10 mg, 5% yield). ¹H NMR (400 MHz, CD₃OD) δ=8.11-8.16 (m, 2H), 7.77 (d, J=2.0 Hz, 1H), 7.57-7.60 (m, 1H)), 7.26-7.40 (m, 3H), 7.07-7.12 (m, 1H), 6.99-7.03 (m, 1H), 6.49 (d, J=5.6 Hz, 1H), 3.67 (s, 2H), 2.93 (bs, 4H), 2.62 (bs, 7H). LCMS: 559.2 [M−H].

### Example IE-23

### 1-(4-((3-chloro-1-pyrrolo[2,3-b]pyridin-4-yl)oxy)-2-fluorophenyl)-3-(3-(difluoromethyl)-4-((4-ethylpiperazin-1-yl)methyl)phenyl)urea

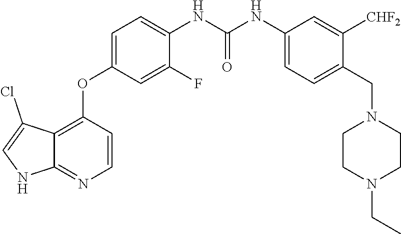 [SMILES: CCN1CCN(Cc2ccc(NC(=O)Nc3ccc(Oc4ccnc5[nH]cc(Cl)c45)cc3F)cc2C(F)F)CC1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.150 g, 0.377 mmol) and 3-(difluoromethyl)-4-((4-ethylpiperazin-1-yl)methyl)aniline (Ie-E14, 0.101 g, 0.377 mmol), and was obtained as a white solid (30 mg, 14% yield). ¹H NMR (400 MHz, CD₃OD) δ=8.11-8.17 (m, 2H), 7.78 (d, J=1.6 Hz, 1H), 7.59-7.61 (m, 1H), 726-7.40 (m, 3H), 7.07-7.12 (m, 1H), 7.00-7.03 (m, 1H), 6.49 (d, J=6.0 Hz, 1H), 3.70 (s, 2H), 3.01-3.03 (m, 6H), 2.66 (bs, 4H), 1.29 (t, J=7.6 Hz, 3H). LCMS: 573.2 [M+H].

### Example IE-24

### 1-(4-((3-chloro-1H-pyrrolo[2,3-b]pyridin-4-yl)oxy)-2-fluorophenyl)-3-(4-(4-methyl-1H-imidazol-1-yl)-3-(trifluoromethyl)phenyl)urea

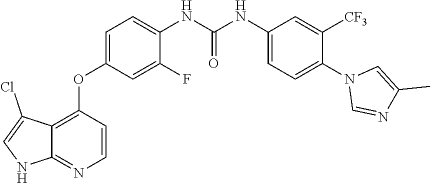 [SMILES: Cc1cn(-c2ccc(NC(=O)Nc3ccc(Oc4ccnc5[nH]cc(Cl)c45)cc3F)cc2C(F)(F)F)cn1]

The title compound was prepared following the general procedure for urea formation (Method Ie-B), starting from phenyl (4-((3-chloro-1H-pyrrolo[2,3-b]pyridin-4-yl)oxy)-2-fluorophenyl)carbamate (Ie-F10, 0.090 g, 0.226 mmol) and 4-(4-methyl-11-imidazol-1-yl)-3-(trifluoromethyl)aniline (Ie-E15, 0.055 g, 0.226 mmol), and was obtained as a white solid (13 mg, 10% yield). ¹H NMR (400 MHz, CD₃OD) δ=9.20 (d, J=0.8 Hz, 1H), 8.25 (d, J=2.4 Hz, 1H), 8.16-8.20 (m, 2H), 7.91-7.94 (m, 1H), 7.66-7.68 (m, 1H), 7.59 (bs, 1H), 7.42 (s, 1H), 7.14-7.18 (m, 1H), 7.06-7.08 (m, 1H), 6.58 (d, J=6.0 Hz, 1H), 2.47 (s, 3H). LCMS: 545.1 [M+H].

### Example IE-25

### 1-(4-((2,3-dichloro-1H-pyrrolo[2,3-b]pyridin-4-yl)oxy)-2-fluorophenyl)-3-(4-((4-methylpiperazin-1-yl)methyl)-3-(trifluoromethyl)phenyl)urea

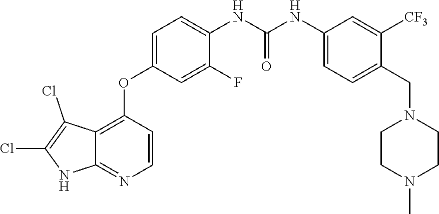 [SMILES: CN1CCN(Cc2ccc(NC(=O)Nc3ccc(Oc4ccnc5[nH]c(Cl)c(Cl)c45)cc3F)cc2C(F)(F)F)CC1]

The title compound was prepared following the general procedure for urea formation (Method Ie-B), starting from phenyl (4-((2,3-dichloro-1H-pyrrolo[2,3-b]pyridin-4-yl)oxy)-2-fluorophenyl)(phenoxycarbonyl)carbamate (Ie-F11, 0.280 g, 0.647 mmol) and 4-((4-methylpiperazin-1-yl)methyl)-3-(trifluoromethyl)aniline (Ie-E16, 0.177 g, 0.647 mmol), and was obtained as a white solid (15 mg, 4% yield). ¹H NMR (400 MHz, CD₃OD) δ=8 11-8.17 (m, 2H), 7.90 (d, J=2.0 Hz, 1H), 7.66-7.72 (m, 2H), 7.08-7.12 (m, 1H), 7.00-7.03 (m, 1H), 6.52 (d, 6.0 Hz, 1H), 3.71 (s, 2H), 2.98 (bs, 4H), 2.66 (bs, 7H). LCMS: 611.1 [M+H].

### Example IE-26

### 1-(4-((3-cyclopropyl-1H-pyrrolo[2,3-b]pyridin-4-yl)oxy)-2-fluorophenyl)-3-(4-((4-methylpiperazin-1-yl)methyl)-3-(trifluoromethyl)phenyl)urea

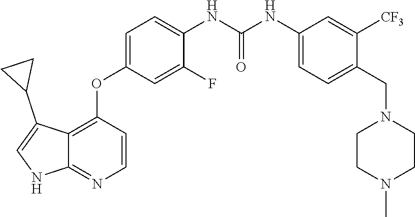 [SMILES: CN1CCN(Cc2ccc(NC(=O)Nc3ccc(Oc4ccnc5[nH]cc(C6CC6)c45)cc3F)cc2C(F)(F)F)CC1]

The title compound was prepared following the general procedure for urea formation (Method Ie-A), starting from 4-((3-cyclopropyl-1H-pyrrolo[2,3-b]pyridin-4-yl)oxy)-2-fluoroaniline (Ie-D3, 0.038 g, 0.134 mmol) and phenyl (4-((4-methylpiperazin-1-yl)methyl)-3-(trifluoromethyl)phenyl)carbamate (Ie-F1, 0.052 g, 0.132 mmol), and was obtained as an off-white solid (3 mg, 4% yield). ¹H NMR (400 MHz, CD₃OD) δ=8.08-8.13 (m, 1H), 8.03-8.04 (m, 1H), 7.91 (d, J=2.0 Hz, 1H), 7.70-7.72 (m, 1H), 7.63-7.65 (m, 1H), 6.97-7.07 (m, 3H), 6.47 (d, J=5.2 Hz, 1H), 3.66 (s, 2H), 2.60-2.63 (m, 8H), 2.40 (s, 3H), 2.10-2.18 (m, 1H), 0.81-0.84 (m, 2H), 0.60-0.62 (m, 2H). LCMS: 583.2 [M+H].

### Example IE-27

### 1-(4-((3-chloro-2-methyl-1H-pyrrolo[2,3-b]pyridin-4-yl)oxy)-2-fluorophenyl)-3-(4-((4-methylpiperazin-1-yl)methyl)-3-(trifluoromethyl)phenyl)urea

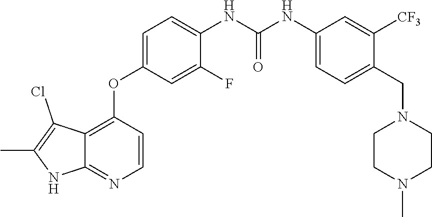 [SMILES: Cc1[nH]c2nccc(Oc3ccc(NC(=O)Nc4ccc(CN5CCN(C)CC5)c(C(F)(F)F)c4)c(F)c3)c2c1Cl]

The title compound was prepared following the general procedure for urea formation (Method Ie-A), starting from 4-((3-chloro-2-methyl-11-pyrrolo[2,3-b]pyridin-4-yl)oxy)-2-fluoroaniline (Ie-D4, 0.070 g, 0.240 mmol) and phenyl (4-((4-methylpiperazin-1-yl)methyl)-3-(trifluoromethyl)phenyl)carbamate (Ie-F1, 0.094 g, 0.240 mmol), and was obtained as an off-white solid (13 mg, 9% yield). ¹H NMR (400 MHz, CD₃OD) δ=8.08-8.13 (m, 1H), 8.02 (d, J=5.6 Hz, 1H), 7.90 (d, J=2.0 Hz, 1H), 7.69-7.72 (m, 1H), 7.63-7.65 (m, 1H), 7.02-7.06 (m, 1H), 6.96-6.98 (m, 1H), 6.48 (d, J=5.6 Hz, 1H), 3.66 (s, 2H), 2.56-2.71 (m, 8H), 2.44 (2s, 6H). LCMS: 591.2 [M+H].

### Example IE-28

### 1-(2-fluoro-4-((3-methyl-1H-pyrrolo[2,3-b]pyridin-4-yl)oxy)phenyl)-3-(4-((4-methylpiperazin-1-yl)methyl)-3-(trifluoromethyl)phenyl)urea

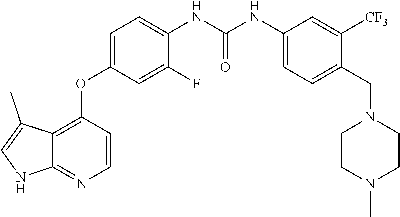 [SMILES: Cc1c[nH]c2nccc(Oc3ccc(NC(=O)Nc4ccc(CN5CCN(C)CC5)c(C(F)(F)F)c4)c(F)c3)c12]

The title compound was prepared following the general procedure for urea formation (Method Ie-A), starting from 2-fluoro-4-((3-methyl-1H-pyrrolo[2,3-b]pyridin-4-yl)oxy)aniline (Ie-D5, 0.060 g, 0.233 mmol) and phenyl (4-((4-methylpiperazin-1-yl)methyl)-3-(trifluoromethyl)phenyl)carbamate (Ie-F1, 0.092 g, 0.233 mmol), and was obtained as an off-white solid (4 mg, 3% yield). ¹H NMR (400 MHz, CD₃OD) δ=8.07-8.12 (m, 1H), 8.02 (d, J=5.6 Hz, 1H), 7.91 (s, 1H), 7.68-7.69 (m, 2H), 6.98-7.07 (m, 3H), 6.42-6.43 (m, 1H), 3.72 (s, 2H), 3.10 (bs, 4H), 2.66-2.74 (m, 7H), 2.43 (s, 3H). LCMS: 557.3 [M+H].

### Example IE-29

### 1-(4-((3-chloro-1H-pyrrolo[2,3-b]pyridin-4-yl)oxy)-2-methylphenyl)-3-(4-((4-methylpiperazin-1-yl))methyl)-3-(trifluoromethyl)phenyl)urea

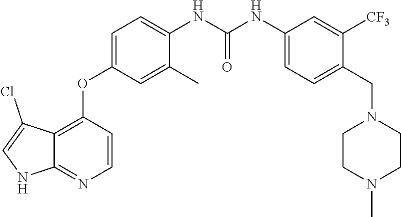 [SMILES: Cc1cc(Oc2ccnc3[nH]cc(Cl)c23)ccc1NC(=O)Nc1ccc(CN2CCN(C)CC2)c(C(F)(F)F)c1]

The title compound was prepared following the general procedure for urea formation (Method Ie-B), starting from phenyl (4-((3-chloro-1H-pyrrolo[2,3-b]pyridin-4-yl)oxy)-2-methylphenyl)(phenoxycarbonyl)carbamate (Ie-F12, 0.090 g, 0.175 mmol) and 4-((4-methylpiperazin-1-yl)methyl)-3-(trifluoromethyl)aniline (Ie-E16, 0.053 g, 0.193 mmol), and was obtained as a white solid (24 mg, 23% yield). ¹H NMR (400 MHz, CD₃OD) δ=8.17 (d, J=6.4 Hz, 1H), 7.92 (s, 1H), 7.80 (d, J=8.8 Hz, 1H), 7.70-7.70 (m, 2H), 7.47 (s, 1H), 7.17 (d, J=2.4 Hz, 1H), 7.09-7.12 (m, 1H), 6.58 (d, J 6.0 Hz, 1H), 3.76 (s, 2H), 3.50-3.50 (m, 4H), 3.15-3.15 (m, 4H), 2.93 (s, 3H), 2.38 (s, 3H). LCMS: 573.3 [M+H].

### Example IE-30

### 1-(4-((2,3-dichloro-1H-pyrrolo[2,3-b]pyridin-4-yl)oxy)-2-methylphenyl)-3-(4-((4-methylpiperazin-1-yl)methyl)-3-(trifluoromethyl)phenyl)urea

### Step 1: Synthesis of 1-(4-((2,3-dichloro-1-((2-(trimethylsilyl)ethoxy)methyl)-1H-pyrrolo[2,3-b]pyridin-4-yl)oxy)-2-methylphenyl)-3-(4-((4-methylpiperazin-1-yl)methyl)-3-(trifluoromethyl)phenyl)urea

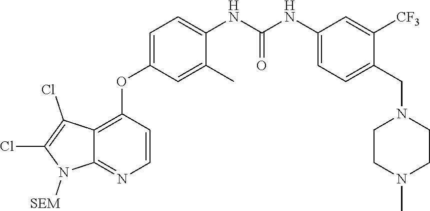 [SMILES: Cc1cc(Oc2ccnc3c2c(Cl)c(Cl)n3COCC[Si](C)(C)C)ccc1NC(=O)Nc1ccc(CN2CCN(C)CC2)c(C(F)(F)F)c1]

The title compound was prepared following the general procedure for urea formation (Method Ie-B), starting from 4-((2,3-dichloro-1-((2-(trimethylsilyl)ethoxy)methyl)-1H-pyrrolo[2,3-b]pyridin-4-yl)oxy)-2-methylaniline (Ie-D7, 0.220 g, 0.491 mmol) and phenyl (4-((4-methylpiperazin-1-yl)methyl)-3-(trifluoromethyl)phenyl)carbamate (Ie-F1, 0.195 g, 0.193 mmol), and was obtained as a white gum (150 mg) which was used without further purification. LCMS: 732.2 [M+H].

### Step 2: Synthesis of 1-(4-((2,3-dichloro-1H-pyrrolo[2,3-b]pyridin-4-yl)oxy)-2-methylphenyl)-3-(4-((4-methylpiperazin-1-yl)methyl)-3-(trifluoroethyl)phenyl)urea

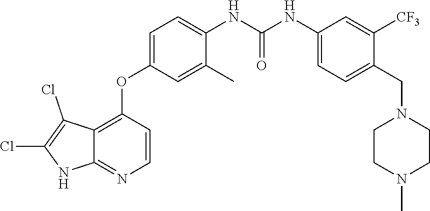 [SMILES: Cc1cc(Oc2ccnc3[nH]c(Cl)c(Cl)c23)ccc1NC(=O)Nc1ccc(CN2CCN(C)CC2)c(C(F)(F)F)c1]

HCl in dioxane (4M, 0.051 mL, 0.203 mmol) was added dropwise to a solution of 1-(4-((2,3-dichloro-1-((2-(trimethylsilyl)ethoxy)methyl)-1H-pyrrolo[2,3-b]pyridin-4-yl)oxy)-2-methylphenyl)-3-(4-((4-methylpiperazin-1-yl)methyl)-3-(trifluoromethyl)phenyl)urea (0.15 g, 0.203 mmol) in DCM (5 mL) at 0° C. and the resulting mixture was stirred at 25° C. for 3 h. Following completion of the reaction (as indicated by UPLC), the reaction mixture was concentrated under reduced pressure to give crude material which was purified by preparative HPLC (eluting with 0.1% TFA in water:ACN), affording the title compound as an off-white solid (37 mg, 30% yield). ¹H NMR (400 MHz, CD₃OD) δ=8.08 (d, J=5.6 Hz, 1H), 7.90 (d, J=1.6 Hz, 1H), 7.70-7.75 (m, 3H), 7.07-7.12 (m, 2H), 6.49 (d, J=5.6 Hz, 1H), 3.76 (s, 2H), 3.43-3.51 (m, 2H), 3.06-3.23 (m, 4H), 2.93 (s, 3H), 2.46-2.49 (m, 2H), 2.36 (s, 3H). LCMS: 607.3 [M+H].

### Biological Example 1

### NEK7 Enzymatic Assay

Casein substrate (from bovine milk, hydrolyzed and partially dephosphorylated mixture of α, β and κ caseins, obtained from Sigma Aldrich, catalogue #C4765, diluted in distilled water to a final concentration of 1 mg/mL) and full-length recombinant human NEK7 (expressed by baculovirus in Sf9 insect cells using a N-terminal GST tag, obtained from SignalChem, catalogue #N09-10G, 0.1 μg/L) were mixed in assay buffer (20 mM Hepes pH 7.5, 10 mM MgCl₂, 1 mM EGTA, 0.02% Brij35, 0.02 mg/ml BSA, 0.1 mM Na₃VO₄, 2 mM DTT, 1% DMSO). Compounds of interest (serial 3-fold dilution in DMSO from 10 μM to 0.5 nM) or vehicle (1% DMSO) were dispensed into the kinase reaction mixture by Acoustic technology (Echo550; nanoliter range). After incubation at room temperature for 20 minutes, the kinase reaction was initiated by addition of [³³P]-ATP (specific activity 10 μCi/μl) and the mixture was incubated at room temperature for 2 hours. The reaction was then stopped by spotting the reaction mixture on strips of phosphocellulose P81 paper. Following washing, the radioactivity of the P81 paper was measured and kinase activity data were expressed as the percent remaining kinase activity in test samples compared to vehicle reactions. IC₅₀ values and curve fits were obtained using Prism (GraphPad Software).

### Biological Example 2

### IL-1β Release Assay

Approximately 1.5 million THP-1 cells were plated in each well of a 6-well TC plate and incubated with 40 nM PMA in RPMI (10% FBS, 1% Penstrep) for 24 hours. The media was then removed and cells were rested in RPMI (10% FBS, 1% Penstrep) for 24 hours after which time the media was removed and cells were pre-treated for 2 hours with various concentrations of compounds of interest (typically serial 3-fold dilution in RPMI+5% FBS, concentrations ranging from 1 μM to 0.5 nM) in RPMI (5% FBS). The media was again removed and cells were incubated with 250 ng/mL LPS and compounds of interest (concentrations as above) in RMPI (5% FBS) for 2 hours. The media was removed for a last time and cells were incubated with 20 μM nigericin and compounds of interest (concentrations as above) in Opti-MEM for 30 minutes. Cell media was then collected and the amount of cleaved IL-1β was determined using a JESS instrument (Protein Simple) and standard protocols. Cleaved Il-1β antibody was obtained from Cell Signaling (catalogue #83186S) and was used at 1:20 dilution in antibody diluent 2. Protein Simple 1× anti-Rabbit HRP secondary antibody was used along with Protein Simple luminol and peroxide for chemiluminescent detection. Primary antibody incubation time was increased from 30 minutes to 60 minutes.

### Biological Example 3

### Assay Data and Comparative Results

Representative compounds of Structure (Ia) were tested for inhibitory activity against NEK7 and IL-1β release according to the procedures described above. Results are given in the following table.

TABLE 2a
Activity of Representative Compounds

| | NEK7 | II-1β release |
|---|---|---|
| No. | IC₅₀ (nM) | IC₅₀ (nM) |
| Ia-1 | *** | − |
| Ia-2 | **** | − |
| Ia-3 | **** | − |
| Ia-4 | *** | − |
| Ia-5 | **** | − |
| Ia-6 | ** | − |
| Ia-7 | **** | − |
| Ia-8 | ***** | − |
| Ia-9 | *** | − |
| Ia-10 | ***** | − |
| Ia-11 | ***** | − |
| Ia-12 | ***** | ++++ |
| Ia-13 | ***** | ++++ |
| Ia-14 | ***** | ++++ |
| Ia-15 | ***** | − |
| Ia-16 | ** | − |
| Ia-17 | *** | − |
| Ia-18 | **** | − |
| Ia-19 | *** | − |
| Ia-20 | ***** | − |
| Ia-21 | ***** | ++++ |
| Ia-22 | **** | − |
| Ia-23 | ***** | − |
| Ia-24 | ***** | − |
| Ia-25 | ***** | ++++ |
| Ia-26 | **** | − |
| Ia-27 | ** | − |
| Ia-28 | * | − |
| Ia-29 | ***** | − |
| Ia-30 | ***** | ++++ |
| Ia-31 | ** | − |
| Ia-32 | ***** | +++ |
| Ia-33 | ** | ++ |
| Ia-34 | * | − |
| Ia-35 | ** | − |
| Ia-36 | ** | − |
| Ia-37 | * | − |
| Ia-38 | * | − |
| Ia-39 | **** | − |
| Ia-40 | **** | − |
| Ia-41 | *** | − |
| Ia-42 | ***** | − |
| Ia-43 | **** | − |
| Ia-44 | **** | − |
| Ia-45 | **** | +++ |
| Ia-48 | *** | − |
| Ia-61 | **** | − |

For NEK7 IC₅₀ activity in Table 2a:
* IC₅₀ greater than 1500 nM
** IC₅₀ range from 501-1500 nM
*** IC₅₀ range from 301-500 nM
**** IC₅₀ range from 151-300 nM
***** IC₅₀ less than 150 nM
For IL-1β IC₅₀ activity in Table 2a:
+ IC₅₀ greater than 1000 nM
++ IC₅₀ range from 301-500 nM
+++ IC₅₀ from 151-300 nM
++++ IC₅₀ less than 150 nM
− denotes a value was not determined

Representative compounds of Structure (Ib) were tested for inhibitory activity against NEK7 and IL-1β release according to the procedures described above. Results are given in the following table.

TABLE 2b
Activity of Representative compounds of Structure (Ib)

| | NEK7 | Il-1β release |
|---|---|---|
| Compound | IC₅₀ (nM) | IC₅₀ (nM) |
| Ib-1 | * | − |
| Ib-2 | * | − |
| Ib-3 | * | − |
| Ib-4 | ** | ++++ |
| Ib-5 | * | +++ |
| Ib-6 | ** | − |
| Ib-7 | ** | − |
| Ib-8 | * | ++ |
| Ib-9 | * | − |
| Ib-10 | * | − |
| Ib-11 | ** | − |
| Ib-12 | ** | ++++ |
| Ib-13 | * | − |
| Ib-14 | ** | + |
| Ib-15 | * | − |
| Ib-16 | * | − |
| Ib-17 | * | − |
| Ib-18 | ** | ++++ |
| Ib-19 | * | − |
| Ib-20 | * | − |
| Ib-21 | * | − |
| Ib-22 | * | − |

For NEK7 IC₅₀ activity in Table 2b:
* IC₅₀ greater than 1500 nM
** IC₅₀ range from 501-1500 nM
*** IC₅₀ range from 301-500 nM
**** IC₅₀ range from 151-300 nM
***** IC₅₀ less than 150 nM
For IL-1β IC₅₀ activity in Table 2b:
+ IC₅₀ greater than 1000 nM
++ IC₅₀ range from 301-500 nM
+++ IC₅₀ from 151-300 nM
++++ IC₅₀ less than 150 nM
− denotes a value was not determined

Representative compounds of Structure (Ic) were tested for inhibitory, activity against NEK7 and IL-1β release according to the procedures described above. Results are given in the following table.

TABLE 2c
Activity of Representative Compounds of Structure (Ic)

| | NEK7 | IL-1β release |
|---|---|---|
| Compound No. | IC₅₀ (nM) | IC₅₀ (nM) |
| Ic-1 | **** | − |
| Ic-2 | ***** | ++++ |
| Ic-3 | ***** | +++ |
| Ic-4 | ***** | − |
| Ic-5 | ***** | ++++ |
| Ic-6 | *** | − |
| Ic-7 | ** | − |
| Ic-8 | ** | − |
| Ic-9 | ***** | +++ |
| Ic-10 | **** | ++++ |
| Ic-11 | *** | − |
| Ic-12 | **** | − |
| Ic-13 | *** | − |
| Ic-14 | **** | ++++ |
| Ic-15 | * | + |
| Ic-16 | * | − |
| Ic-17 | ***** | ++++ |
| Ic-18 | * | − |
| Ic-19 | *** | − |
| Ic-20 | * | − |
| Ic-21 | * | − |
| Ic-22 | * | − |
| Ic-23 | *** | − |
| Ic-24 | ** | − |
| Ic-25 | ** | − |
| Ic-26 | *** | − |
| Ic-27 | ***** | − |
| Ic-28 | **** | − |
| Ic-29 | *** | − |
| Ic-30 | **** | − |
| Ic-31 | *** | − |
| Ic-32 | ***** | − |
| Ic-33 | ***** | − |
| Ic-34 | ***** | ++++ |
| Ic-35 | **** | +++ |
| Ic-36 | ** | − |
| Ic-37 | ***** | ++++ |
| Ic-38 | ** | ++ |
| Ic-39 | ***** | ++++ |
| Ic-40 | * | − |
| Ic-41 | * | − |
| Ic-42 | ** | − |
| Ic-43 | * | − |
| Ic-44 | ** | − |
| Ic-45 | **** | − |
| Ic-46 | ***** | − |
| Ic-47 | ***** | − |
| Ic-48 | * | − |
| Ic-49 | * | − |
| Ic-50 | * | − |
| Ic-51 | ***** | − |
| Ic-52 | ***** | − |
| Ic-53 | *** | − |
| Ic-54 | * | − |
| Ic-55 | * | − |
| Ic-56 | *** | − |
| Ic-57 | ***** | − |
| Ic-58 | ** | − |
| Ic-59 | * | − |
| Ic-60 | * | − |
| Ic-61 | ***** | − |
| Ic-62 | * | − |

For NEK7 IC₅₀ activity in Table 2c:
* IC₅₀ greater than 1500 nM
** IC₅₀ range from 501-1500 nM
*** IC₅₀ range from 301-500 nM
**** IC₅₀ range from 151-300 nM
***** IC₅₀ less than 150 nM
For IL-1β IC₅₀ activity in Table 2c:
+ IC₅₀ greater than 1000 nM
++ IC₅₀ range from 301-500 nM
+++ IC₅₀ from 151-300 nM
++++ IC₅₀ less than 150 nM
− denotes a value was not determined

Representative compounds of Structure (Id) were tested for inhibitory activity against NEK7 and IL-1β release according to the procedures described above. Results are given in the table below.

TABLE 2d
Activity of Representative compounds of Structure (Id)

| | | IL-1β | | | IL-1β |
|---|---|---|---|---|---|
| | NEK7 | release | | NEK7 | release |
| | IC₅₀ | IC₅₀ | | IC₅₀ | IC₅₀ |
| Compound | (nM) | (nM) | Compound | (nM) | (nM) |
| Id-1 | * | − | Id-2 | * | − |
| Id-3 | ** | + | Id-4 | **** | − |
| Id-5 | ***** | − | Id-6 | * | − |
| Id-7 | * | − | Id-8 | *** | − |
| Id-9 | ***** | − | Id-10 | **** | ++++ |
| Id-11 | * | − | Id-12 | − | − |
| Id-13 | − | − | Id-14 | ***** | − |
| Id-15 | ***** | − | Id-16 | * | − |
| Id-17 | − | − | Id-18 | ***** | ++++ |
| Id-19 | ***** | − | Id-20 | ***** | − |
| Id-21 | ***** | − | Id-22 | * | − |
| Id-23 | * | − | Id-24 | ***** | ++++ |
| Id-25 | *** | − | Id-26 | * | − |
| Id-27 | ***** | ++++ | Id-28 | ***** | ++++ |
| Id-29 | **** | − | Id-30 | ***** | ++++ |
| Id-31 | ** | ++ | Id-32 | * | − |
| Id-33 | ***** | ++++ | Id-34 | **** | − |
| Id-35 | *** | − | Id-36 | ***** | − |
| Id-37 | * | − | Id-38 | * | − |
| Id-39 | − | − | Id-40 | ***** | − |
| Id-41 | ***** | − | Id-42 | ***** | − |
| Id-43 | ***** | − | Id-44 | ***** | − |
| Id-45 | ** | − | Id-46 | ***** | − |
| Id-47 | * | − | Id-48 | **** | + |
| Id-49 | ***** | ++++ | Id-50 | *** | − |
| Id-51 | ** | − | Id-52 | * | − |
| Id-53 | * | − | Id-54 | ***** | − |
| Id-55 | * | − | Id-56 | * | − |
| Id-57 | * | − | Id-58 | * | − |
| Id-59 | * | − | Id-60 | * | − |
| Id-61 | *** | − | Id-62 | ***** | − |
| Id-63 | ***** | − | Id-64 | ** | − |
| Id-65 | ***** | − | Id-66 | ***** | − |
| Id-67 | ***** | − | Id-68 | ***** | − |
| Id-69 | **** | − | Id-70 | ***** | − |
| Id-71 | ***** | − | | | |

For NEK7 IC₅₀ activity in Table 2d:
* IC₅₀ greater than 1500 nM
** IC₅₀ range from 501-1500 nM
*** IC₅₀ range from 301-500 nM
**** IC₅₀ range from 151-300 nM
***** IC₅₀ less than 150 nM
For IL-1β IC₅₀ activity in Table 2d:
+ IC₅₀ greater than 1000 nM
++ IC₅₀ range from 301-500 nM
+++ IC₅₀ from 151-300 nM
++++ IC₅₀ less than 150 nM
− denotes a value was not determined

Representative compounds of Structure (Ie) were tested for inhibitory activity against NEK7 and IL-1β release according to the procedures described above. Results are given in the table below.

TABLE 2e
Activity of Representative compounds of Structure (Ie)

| | NEK7 | IL-1β release |
|---|---|---|
| Compound | IC₅₀ (nM) | IC₅₀ (nM) |
| Ie-1 | **** | ++++ |
| Ie-2 | * | − |
| Ie-3 | ** | − |
| Ie-4 | * | − |
| Ie-5 | * | − |
| Ie-6 | * | − |
| Ie-7 | * | − |
| Ie-8 | * | − |
| Ie-9 | ** | ++++ |
| Ie-10 | *** | − |
| Ie-11 | * | − |
| Ie-12 | * | − |
| Ie-13 | ** | − |
| Ie-14 | * | − |
| Ie-15 | * | − |
| Ie-16 | **** | ++++ |
| Ie-17 | * | − |
| Ie-18 | * | − |
| Ie-19 | ** | − |
| Ie-20 | *** | ++++ |
| Ie-21 | * | − |
| Ie-22 | ** | − |
| Ie-23 | ** | − |
| Ie-24 | *** | − |
| Ie-25 | *** | − |
| Ie-26 | ** | ++++ |
| Ie-27 | *** | ++++ |
| Ie-28 | **** | +++ |
| Ie-29 | * | − |
| Ie-30 | * | − |
| Ie-31 | * | − |
| Ie-32 | * | − |
| Ie-33 | * | − |
| Ie-35 | * | − |
| Ie-40 | * | − |
| Ie-41 | * | − |
| Ie-42 | * | − |
| Ie-43 | * | − |

For NEK7 IC₅₀ activity in Table 2e:
* IC₅₀ greater than 1500 nM
** IC₅₀ range from 501-1500 nM
*** IC₅₀ range from 301-500 nM
**** IC₅₀ range from 151-300 nM
***** IC₅₀ less than 150 nM
For IL-1β IC₅₀ activity in Table 2e:
+ IC₅₀ greater than 1000 nM
++ IC₅₀ range from 301-500 nM
+++ IC₅₀ from 151-300 nM
++++ IC₅₀ less than 150 nM
− denotes a value was not determined

The above data illustrate NEK7 small molecule inhibitor compounds binding via a type 2 binding mode. It should be noted that each of the above compounds have at least one of the following features: (i) a hinge-binding element, (ii) a flexible linker, (iii) a urea-type linker, and (iv) a hydrophobic back pocket group.

### Biological Example 4

### Comparative Data for Type 1.5 Binding Compounds

The following comparative compounds were tested and showed potent NEK7 inhibitory activity but lack desirable IL-1β release IC₅₀ activity. Without wishing to be bound by theory, it is thought that compounds of the present disclosure (i.e., Compounds of Structure (I), (Ia), (Ib), (Ic), (Id), (Ie), and (If)) bind via a Type 2 binding mode and the following comparative compounds bind via a Type 1.5 binding mode. This result is surprising due to the fact that representative compounds of Structure (II) have similar structural features to compounds of the present disclosure. It is possible that compounds of Structure (1H) bind via a Type 1.5 binding mode because they lack a desirable urea-type linker. That is, at the position corresponding to the urea-type linker of compounds of the present disclosure, compounds of Structure (II) have a —C(═O)CH₂— linker. This replacement linker lacks the ability to form a bidentate interaction with GLU82 of NEK7 which requires hydrogen bond donors only found in the urea-type linkers. Additionally, when combined with a bicyclic indoline linker the resulting indoline-amide structure features a shorter length and increased rigidity when compared to the more flexible monocyclic linker and urea-type linker found in compounds of Structure (I). This could further disrupt the ability of compounds of Structure (II) to accommodate the Type 2 DLG-out conformation of NEK7 and instead induce a Type 1.5 conformation, forcing the DLG motif of the activation loop closer to the hinge-binding region and bringing it in the DLG-in position, preventing access to the allosteric back pocket.

Representative compounds of Structure (II) (i.e., comparative compounds) were tested for inhibitory activity against NEK7 and IL-1β release according to the procedures described above. Results are given in the table below.

TABLE I-II
Reresentative compounds of Structure (II)

| Compound | Structure | Name |
|---|---|---|
| II-1 | 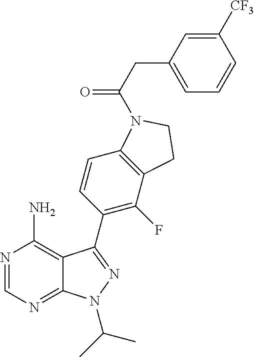 [SMILES: CC(C)n1nc(-c2ccc3c(c2F)CCN3C(=O)Cc2cccc(C(F)(F)F)c2)c2c(N)ncnc21] | l -(5-(4 -amino-1-isopropyl-1H- pyrazolo[3,4-d]pyrimidin-3-yl)- 4-fluoroindolin-1-yl)-2-(3- (trifluoromethyl)phenyl)ethan- 1-one |
| II-2 | 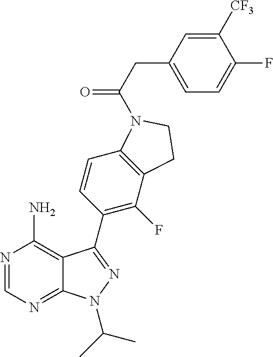 [SMILES: CC(C)n1nc(-c2ccc3c(c2F)CCN3C(=O)Cc2ccc(F)c(C(F)(F)F)c2)c2c(N)ncnc21] | 1-(5-(4-amino-1-isopropyl-1H- pyrazol[3,4-d]pyrimidin-3-yl)- 4-fluoroindolin-1-yl)-2-(4- fluoro-3- (trifluoromethyl)phenyl)ethan- 1-one |
| II-3 | 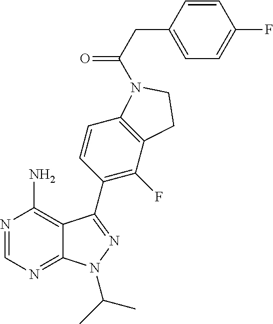 [SMILES: CC(C)n1nc(-c2ccc3c(c2F)CCN3C(=O)Cc2ccc(F)cc2)c2c(N)ncnc21] | 1-(5-(4-amino-1-isopropyl-1H- pyrazolo[3,4-d]pyrimidin-3-yl)- 4-fluoroindolin-1-yl)-2-(4- fluoro-3- (trifluoromethyl)phenyl)ethan- 1-one |
| II-4 | 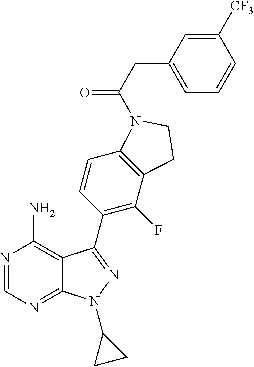 [SMILES: Nc1ncnc2c1c(-c1ccc3c(c1F)CCN3C(=O)Cc1cccc(C(F)(F)F)c1)nn2C1CC1] | 1-(5-(4-amino-1-cyclopropyl- 1H-pyrazolo[3,4-d]pyrimidin-3- yl)-4-fluoroindolin-1-yl)-2-(3- (trifluoromethyl)phenyl)ethan- 1-one |
| II-5 | 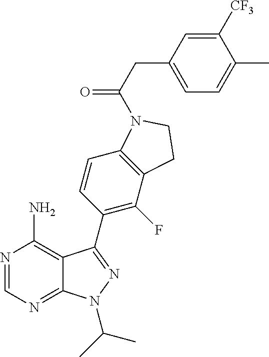 [SMILES: Cc1ccc(CC(=O)N2CCc3c2ccc(-c2nn(C(C)C)c4ncnc(N)c24)c3F)cc1C(F)(F)F] | 1-(5-(4-amino01-isopropyl-1H- pyrazolo[3,4-d]pyrimidin-3-yl)- 4-fluoroindolin-1-yl)-2-(4- methyl-3- (trifluoromethyl)phenyl)ethan- 1-one |
| II-6 | 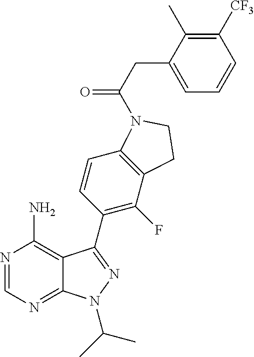 [SMILES: Cc1c(CC(=O)N2CCc3c2ccc(-c2nn(C(C)C)c4ncnc(N)c24)c3F)cccc1C(F)(F)F] | 1-(5-(4-amino-1-isopropyl-1H- pyrazolo[3,4-d]pyrimidin-3-yl)- 4-fluoroindolin-l-yl)-2-(2- methyl-3- (trifluoromethyl)phenyl)ethan- 1-one |
| II-7 | 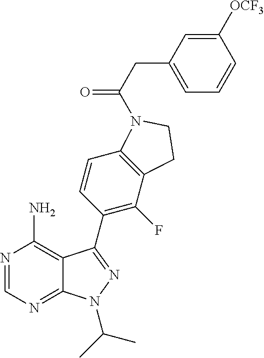 [SMILES: CC(C)n1nc(-c2ccc3c(c2F)CCN3C(=O)Cc2cccc(OC(F)(F)F)c2)c2c(N)ncnc21] | 1-(5-(4-amino-1-isopropyl-1H- pyrazolo[3,4-d]pyrimidin-3-yl)- 4-fluoroindolin-1-yl)-2-(3- (trifluoromethoxy)phenyl)ethan- 1-one |
| II-8 | 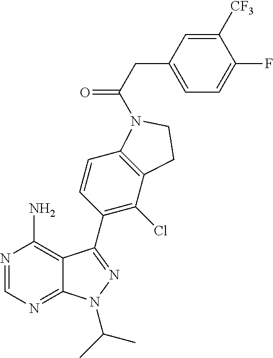 [SMILES: CC(C)n1nc(-c2ccc3c(c2Cl)CCN3C(=O)Cc2ccc(F)c(C(F)(F)F)c2)c2c(N)ncnc21] | 1-(5-(4-amino-1-isopropyl-1H- pyrazolo[3,4-d]pyrimidin-3-yl)- 4-chloroindolin-1-yl)-2-(4- fluoro-3- (trifluoromethyl)phenyl)ethan- 1-one |
| II-9 | 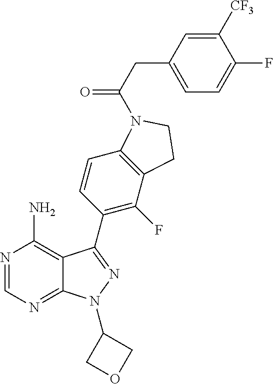 [SMILES: Nc1ncnc2c1c(-c1ccc3c(c1F)CCN3C(=O)Cc1ccc(F)c(C(F)(F)F)c1)nn2C1COC1] | 1-(5-(4-amino-1-(oxetan-3-yl)- 1H-pyrazolo[3,4-d]pyrimidin-3- yl)-4-fluoroindolin-1-yl)-2-(4- fluoro-3- (trifluoromethyl)phenyl)ethan- 1-one |
| II-10 | 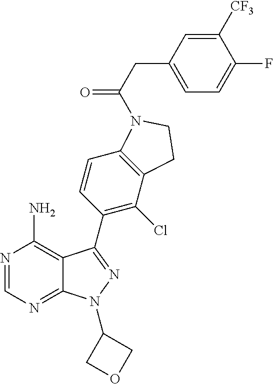 [SMILES: Nc1ncnc2c1c(-c1ccc3c(c1Cl)CCN3C(=O)Cc1ccc(F)c(C(F)(F)F)c1)nn2C1COC1] | 1-(5-(4-amino-1-(oxetan-3-yl)- 1H-pyrazolo[3,4-d]pyrimidin-3- yl)-4-chloroindolin-1-yl)-2-(4- fluoro-3- (trifluoromethyl)phenyl)ethan- 1-one |
| II-11 | 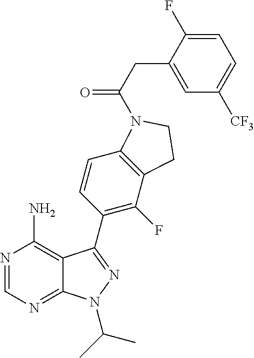 [SMILES: CC(C)n1nc(-c2ccc3c(c2F)CCN3C(=O)Cc2cc(C(F)(F)F)ccc2F)c2c(N)ncnc21] | 1-(5-(4-amino-1-isopropyl-1H- pyrazolo[3,4-d]pyrimidin-3-yl)- 4-fluoroindolin-l-yl)-2-(2- fluoro-5- (trifluoromethyl)phenyl)ethan- 1-one |
| II-12 | 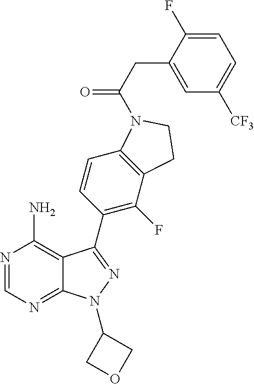 [SMILES: Nc1ncnc2c1c(-c1ccc3c(c1F)CCN3C(=O)Cc1cc(C(F)(F)F)ccc1F)nn2C1COC1] | 1-(5-(4-amino-1-(oxetan-3-yl)- 1H-pyrazolo[3,4-d]pyrimidin-3- yl)-4-fluoroindolin-1-yl)-2-(2- fluoro-5- (trifluoromethyl)phenyl)ethan- 1-one |
| II-13 | 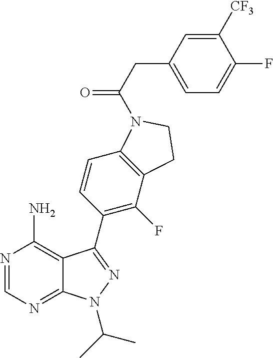 [SMILES: CC(C)n1nc(-c2ccc3c(c2F)CCN3C(=O)Cc2ccc(F)c(C(F)(F)F)c2)c2c(N)ncnc21] | 1-(5-(4-amino-7-isopropy]-7H- pyrrolo[2,3-d]pyrimidin-5-yl)- 4-fluoroindolin-l-yl)-2-(4- fluoro-3- (trifluoromethyl)phenyl)ethan- 1-one |
| II-14 | 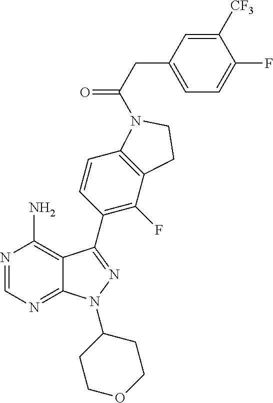 [SMILES: Nc1ncnc2c1c(-c1ccc3c(c1F)CCN3C(=O)Cc1ccc(F)c(C(F)(F)F)c1)nn2C1CCOCC1] | 1-(5-(4-amino-1-(tetrahydro- 2H-pyran-4-yl)-1H- pyrazolo[3,4-d]pyrimidin-3-yl)- 4-fluoroindolin-1-yl)-2-(4- fluoro-3- (trifluromethyl)phenyl)ethan- 1-one |
| II-15 | 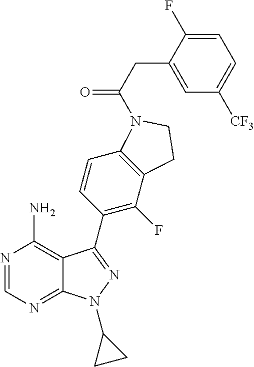 [SMILES: Nc1ncnc2c1c(-c1ccc3c(c1F)CCN3C(=O)Cc1cc(C(F)(F)F)ccc1F)nn2C1CC1] | 1-(5-(4-amino-1-cylcopropyl- 1H-pyrazol[3,4-d]pyrimidin-3- yl)-4-chloroindolin-1-yl)-2-(2- fluoro-5- (triflouromethyl)phenyl)ethan- 1-one |
| II-16 | 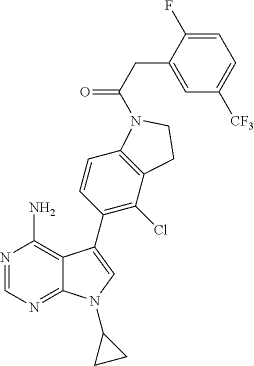 [SMILES: Nc1ncnc2c1c(-c1ccc3c(c1Cl)CCN3C(=O)Cc1cc(C(F)(F)F)ccc1F)cn2C1CC1] | 1-(5-(4-amino-7-cyclopropyl- 7H-pyrrolo[2,3-d]pyrimidin-5- yl)-4-chloroindolin-1-yl)-2-(2- fluoro-5- (trifluoromethyl)phenyl)ehtan- 1-one |
| II-17 | 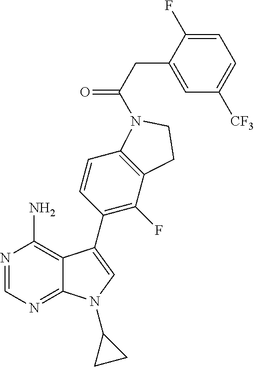 [SMILES: Nc1ncnc2c1c(-c1ccc3c(c1F)CCN3C(=O)Cc1cc(C(F)(F)F)ccc1F)cn2C1CC1] | 1-(5-(4-amino-7-cyclopropyl- 7H-pyrrolo[2,3-d]pyrimidin-5- yl)-4-fluoroindolin-1-yl)-2-(2- fluoro-5- (trifluoromethyl)phenyl)ethan- 1-one |
| II-18 | 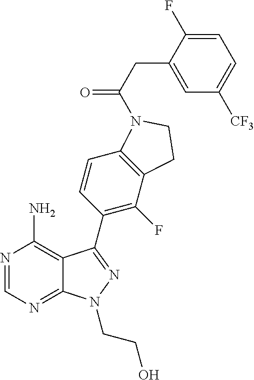 [SMILES: Nc1ncnc2c1c(-c1ccc3c(c1F)CCN3C(=O)Cc1cc(C(F)(F)F)ccc1F)nn2CCO] | 1-(5-(4-amino-7-(2- hydroxyethyl)-7H-pyrrolo[2,3- d]pyrimidin-5-yl)-4- fluoroindolin-1-yl)-2-(2- fluoro-5- (trifluoromethyl)phenyl)ethan- 1-one |

TABLE 2-II
Activity of Representative compounds of Structure (II)

| | Nek7 | IL-1β release |
|---|---|---|
| Compound | IC₅₀ (nM) | IC₅₀ (nM) |
| II-1 | *** | − |
| II-2 | **** | − |
| II-3 | * | − |
| II-4 | **** | − |
| II-5 | *** | − |
| II-6 | * | − |
| II-7 | **** | − |
| II-8 | ** | − |
| II-9 | ** | − |
| II-10 | **** | − |
| II-11 | ***** | + |
| II-12 | ***** | + |
| II-13 | **** | − |
| II-14 | ***** | + |
| II-15 | ***** | + |
| II-16 | ***** | + |
| II-17 | ***** | + |
| II-18 | ***** | − |

For NEK7 IC₅₀ activity in Table 2-II:
* IC₅₀ greater than 1500 nM
** IC₅₀ range from 501-1500 nM
*** IC₅₀ range from 301-500 nM
**** IC₅₀ range from 151-300 nM
***** IC₅₀ less than 150 nM
For IL-1β IC₅₀ activity in Table 2-II:
+ IC₅₀ greater than 1000 nM
++ IC₅₀ range from 301-500 nM
+++ IC₅₀ from 151-300 nM
++++ IC₅₀ less than 150 nM
− denotes a value was not determined

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Application Ser. No. 63/022,159 filed May 8, 2020, U.S. Provisional Application Ser. No. 63/036,244 filed Jun. 8, 2020, U.S. Provisional Application Ser. No. 63/141,370 filed Jan. 25, 2021. U.S. Provisional Application Ser. No. 63/167,523 filed Mar. 29, 2021, U.S. Provisional Application Ser. No. 63/170,776 filed Apr. 5, 2021, U.S. Provisional Application Ser. No. 63/170,761 filed Apr. 5, 2021. U.S. Provisional Application Ser. No. 63/170,708 filed Apr. 5, 2021. U.S. Provisional Application Ser. No. 63/178,385 filed Apr. 2-2, 2021, U.S. Provisional Application Ser. No. 63/185,257 filed May 6, 2021, U.S. Provisional Application Ser. No. 63/185,267 filed May 6, 2021, U.S. Provisional Application Ser. No. 63/185,274 filed May 6, 2021, U.S. Provisional Application Ser. No. 63/185,282 filed May 6, 2021, U.S. Provisional Application Ser. No. 63/185,286 filed May 6, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating or preventing a disease or disorder, the method comprising administering a NEK7 small molecule inhibitor compound to a subject in need thereof, the NEK7 small molecule inhibitor compound comprising at least one of the following features:
i. a hinge-binding element comprising at least one hydrogen donor and at least one hydrogen acceptor;
ii. a flexible linker;
iii. a urea-type linker; or
iv. a hydrophobic back pocket group,
wherein the disease or disorder is a NLRP3-mediated disorder.

2-6. (canceled)

7. The method of claim 1, wherein the hinge-binding element has the following structure: 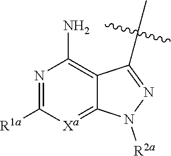
wherein:
X^{a} is N or CH;
R^{1a} is H or C₁-C₆ alkyl; and
R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl.

8-10. (canceled)

11. The method of claim 1, wherein the hinge-binding element has the following structure: 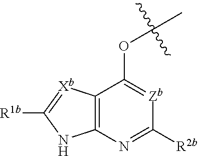
wherein:
X^{b} is N or CR^{10b};
Z^{b} is N or CR^{11b}; and
R^{1b}, R^{2b}, R^{10b}, and R^{11b} are each independently H, halo, C₁-C₆ alkyl, or C₃-C₈ cycloalkyl.

12-17. (canceled)

18. The method of claim 1, wherein the hinge-binding element has the following structure: 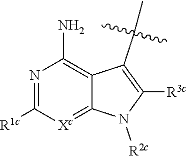
wherein:
X^{c} is CH or N;
R^{1c} is H or C₁-C₆ alkyl;
R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl; and
R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl and C₁-C₆ alkoxy.

19-23. (canceled)

24. The method of claim 1, wherein the hinge-binding element has the following structure: 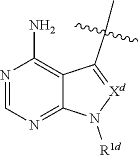
wherein:
X^{d} is N or CR^{4d};
R^{1d} is C₁-C₆ alkyl, C₁-C₆ hydroxylalkyl, C₃-C₁₀ cycloalkyl, or 3-10 membered heterocyclyl; and
R^{4d} is H, C₁-C₆ alkyl, C₁-C₆ haloalkyl, or C₃-C₈ cycloalkyl.

25-27. (canceled)

28. The method of claim 1, wherein the hinge-binding element has the following structure: 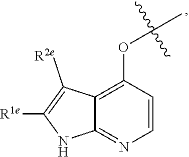
wherein:
R^{1e} is H, halo, C₁-C₆ alkyl, C₃-C₁₀ cycloalkyl, or 3-10 membered heterocyclyl; and
R^{2e} is H, halo, C₁-C₆ alkyl, C₁-C₆ haloalkyl, C₃-C₁₀ cycloalkyl, or 3-10 membered heterocyclyl.

29-33. (canceled)

34. The method of claim 1, wherein the flexible linker has one of the following structures:

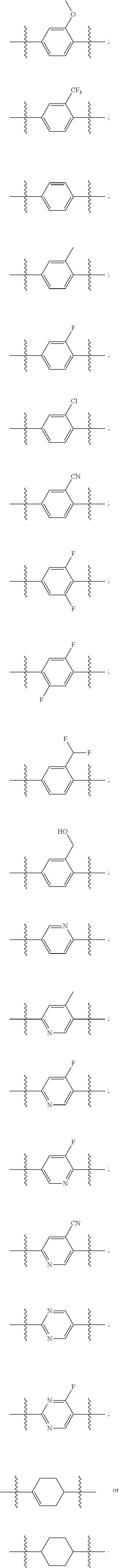

35. The method of claim 1, wherein the urea-type linker comprises the following structure: 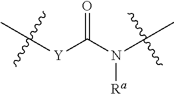
wherein:
Y is C(R^{c})(R^{d}), or NR^{b};
R^{a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl;
R^{b} is H, C₁-C₆ alkyl, C₂-C₆ alkenyl, C₂-C₆ alkynyl, C₃-C₈ cycloalkyl, or C₁-C₆ hydroxylalkyl;
R^{c} is H, C₁-C₆ alkyl, C₂-C₆ alkenyl, C₂-C₆ alkynyl, or C₁-C₆ hydroxylalkyl; and
R^{d} is H, OH, C₁-C₆ alkyl, C₂-C₆ alkenyl, C₂-C₆ alkynyl, or C₁-C₆ hydroxylalkyl.

36-37. (canceled)

38. The method of claim 1, wherein the hydrophobic back pocket group has one of the following structures:

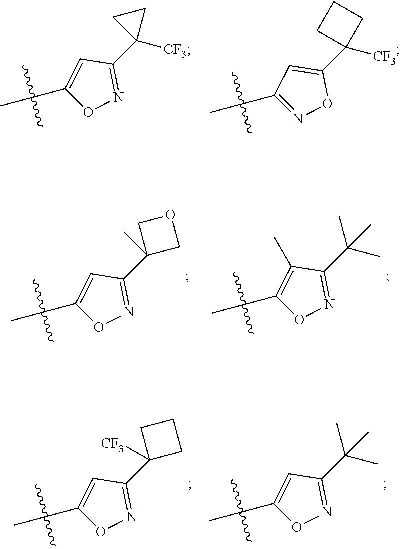

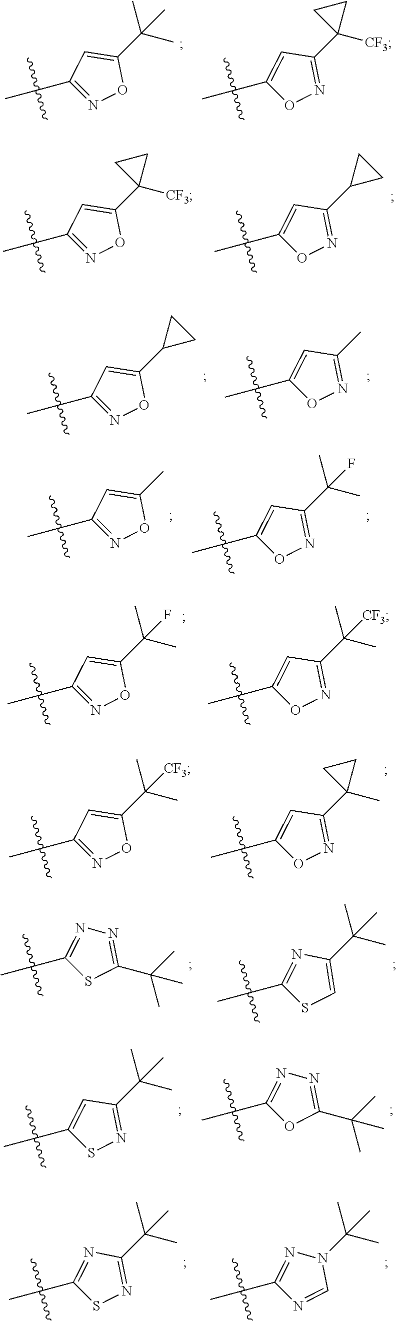

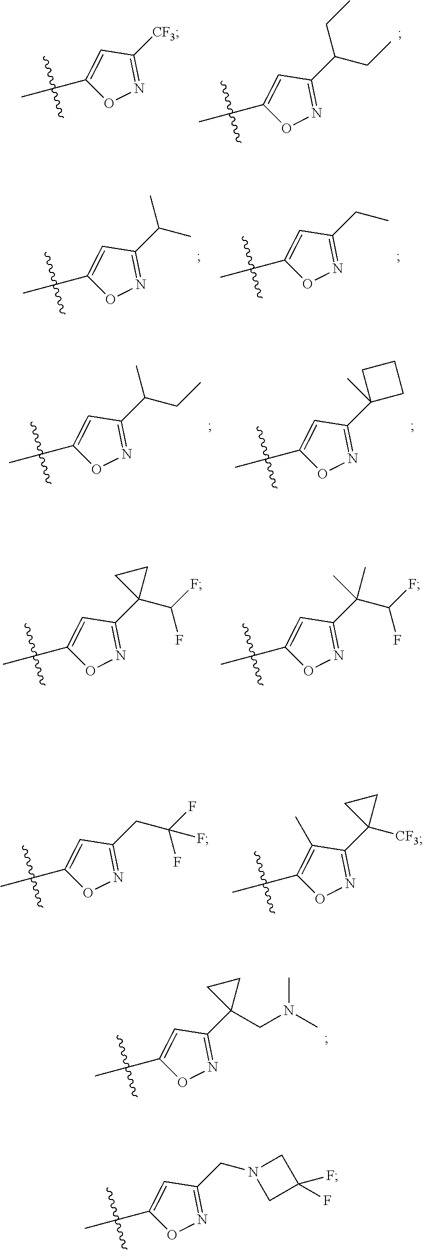

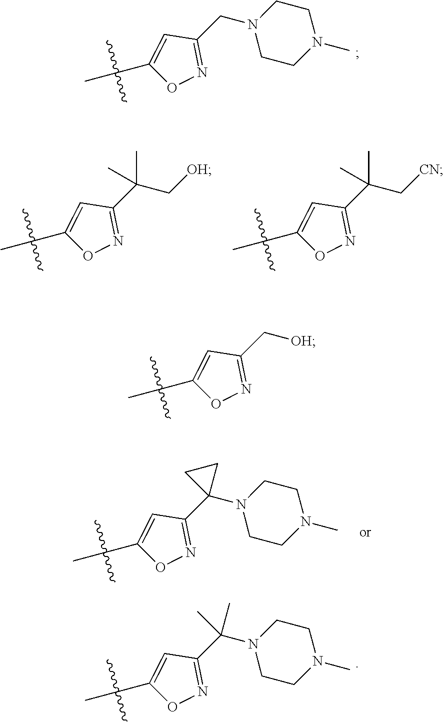 [SMILES: *c1cc(CN2CCN(C)CC2)no1 ; *c1cc(C(C)(C)CO)no1 ; *c1cc(C(C)(C)CC#N)no1 ; *c1cc(CO)no1 ; *c1cc(C2(N3CCN(C)CC3)CC2)no1 ; *c1cc(C(C)(C)N2CCN(C)CC2)no1]

39. The method of claim 1, wherein hydrophobic back pocket group has the following structure: 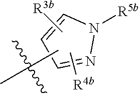
wherein:
R^{3b} and R^{4b} are each independently H, halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl, provided that R² and R^{2b} are not both H; and
R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy.

40-44. (canceled)

45. The method of claim 1, wherein the hydrophobic back pocket group has the following structure: 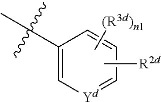
wherein:
Y^{d} is N or CH;
R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;
R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl; and
n1 is 0, 1, 2, 3, or 4.

46-50. (canceled)

51. The method of claim 1, wherein the hydrophobic back pocket group has the following structure: 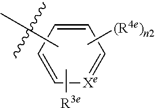
wherein:
X^{e} is N or CH;
R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;
R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and
n2 is 0, 1, 2, 3, or 4.

52-57. (canceled)

58. The method of claim 1, wherein the NEK7 small molecule inhibitor compound comprises each of the following features:
i. a hinge-binding element comprising at least one hydrogen donor and at least one hydrogen acceptor;
ii. a flexible linker;
iii. a urea-type linker; or
iv. a hydrophobic back pocket group.

59. The method of claim 1, wherein the disorder is selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions.

60. The method of claim 1, wherein the disorder is selected from type II diabetes, atherosclerosis, Alzheimer's disease, aging, fatty liver, metabolic syndrome, asthma, psoriasis, obesity, acute and chronic tissue damage caused by infection, gout, arthritis, macular degeneration, enteritis, hepatitis, peritonitis, silicosis, UV-induced skin sunburn, contact hypersensitivity, sepsis, cancer, neurodegenerative disease, multiple sclerosis, and Muckle-Wells syndrome.

61. The method of claim 1, wherein the NEK7 small molecule inhibitor compound has the following Structure (I): 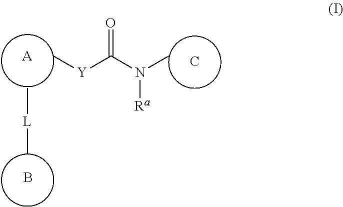
wherein:
A is cycloalkyl, heterocyclyl, aryl, or heteroaryl;
B is a heteroaryl ring;
C is aryl or heteroaryl;
L is a direct bond or —O—;
Y is C(R^{c})(R^{d}), or NR^{b};
R^{a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl;
R^{b} is H, C₁-C₆ alkyl, C₂-C₆ alkenyl, C₂-C₆ alkynyl, C₃-C₈ cycloalkyl, or C₁-C₆ hydroxylalkyl;
R^{c} is H, C₁-C₆ alkyl, C₂-C₆ alkenyl, C₂-C₆ alkynyl, or C₁-C₆ hydroxylalkyl; and
R^{d} is H, OH, C₁-C₆ alkyl, C₂-C₆ alkenyl, C₂-C₆ alkynyl, or C₁-C₆ hydroxylalkyl.

62-103. (canceled)

104. The method of claim 1, wherein the NEK7 small molecule inhibitor compound has the following Structure (Ia): 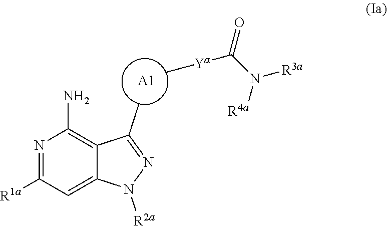
or a pharmaceutically acceptable salt, stereoisomer or prodrug thereof, wherein:
A1 is C₆-C₁₀ aryl, C₃-C₁₀ cycloalkyl, 3-10 membered heterocyclyl or 5-6 membered monocyclic heteroaryl, each of which is optionally substituted with one or more R⁵;
X^{a} is N or CH;
Y^{a} is CHOH or NH;
R^{1a} is H or C₁-C₆ alkyl;
R^{2a} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;
R^{3a} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from amino, halo, cyano, C₁-C₆ alkyl, C₂-C₆ alkenyl, C₂-C₆ alkynyl, C₁-C₆ haloalkyl, C₃-C₈ cycloalkyl, C₃-C₈ alkylcycloalkyl, C₃-C₈ haloalkylcycloalkyl, C₃-C₈ aminylalkylcycloalkyl, C₁-C₆ cyanoalkyl, C₁-C₆ aminyl, C₁-C₆ hydroxylalkyl, 3-8 membered heterocyclyl, 3-8 membered heterocyclylalkyl, 3-8 membered heterocyclylcycloalkyl, 3-8 membered haloheterocyclyl, 3-8 membered haloheterocyclylalkyl, C₃-C₈ halocycloalkyl and C₃-C₈ halocycloalkylalkyl, and combinations thereof;
R^{4a} is H, C₁-C₆ alkyl, C₂-C₆ alkenyl, C₂-C₆ alkynyl, C₃-C₈ cycloalkyl, 3-8 membered heterocyclyl, C₆-C₁₀ aryl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl and C₁-C₆ alkoxy; and
R^{5a} is, at each occurrence, independently halo, cyano, C₁-C₆ alkyl, C₁-C₆ hydroxylalkyl or C₁-C₆ haloalkyl.

105. (canceled)

106. The method of claim 1, wherein the NEK7 small molecule inhibitor compound has the following Structure (Ib): 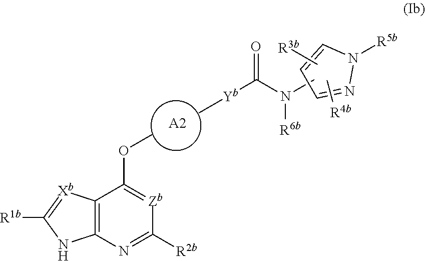
or a pharmaceutically acceptable salt, stereoisomer or prodrug thereof, wherein:
A2 is C₆-C₁₀ aryl, C₃-C₁₀ cycloalkyl, 3-10 membered heterocyclyl, or 5-6 membered monocyclic heteroaryl, each of which is optionally substituted with one or more R^{7b};
X^{b} is N or CR^{10b};
Y^{b} is C(R^{8b})(R^{9b}) or NR^{8b};
Z^{b} is N or CR^{11b};
R^{1b}, R^{2b}, R^{10b}, and R^{11b} are each independently H, halo, C₁-C₆ alkyl, or C₃-C₈ cycloalkyl;
R^{3b} and R^{4b} are each independently H, halo, cyano, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₈ cycloalkyl, or C₃-C₈ halocycloalkyl, provided that R² and R^{2b} are not both H;
R^{5b} is C₁-C₆ alkyl, C₂-C₆ alkenyl, C₂-C₆ alkynyl, C₃-C₈ cycloalkyl, 3-10 membered heterocyclyl, heteroaryl, or aryl, each of which is optionally substituted with one or more substituents selected from halo, hydroxyl, cyano, aminyl, C₁-C₆ alkyl, C₂-C₆ alkenyl, C₂-C₆ alkynyl, and C₁-C₆ alkoxy;
R^{6b} is H, C₁-C₆ alkyl, C₂-C₆ alkenyl, C₂-C₆ alkynyl, C₃-C₈ cycloalkyl, 3-8 membered heterocyclyl, C₆-C₁₀ aryl, or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, and C₁-C₆ alkoxy;
R^{7b} is, at each occurrence, independently halo, C₁-C₆ alkyl, C₂-C₆ alkenyl, C₂-C₆ alkynyl, or C₁-C₆ haloalkyl;
R^{8b} is H, C₁-C₆ alkyl, C₂-C₆ alkenyl, C₂-C₆ alkynyl, or C₁-C₆ hydroxylalkyl; and
R^{9b} is H, OH, C₁-C₆ alkyl, C₂-C₆ alkenyl, C₂-C₆ alkynyl, or C₁-C₆ hydroxylalkyl.

107. (canceled)

108. The method of claim 1, wherein the NEK7 small molecule inhibitor compound has the following Structure (Ic): 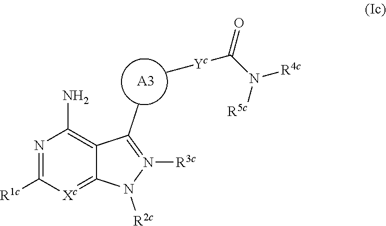
or a pharmaceutically acceptable salt, stereoisomer or prodrug thereof, wherein:
A3 is C₆-C₁₀ aryl, C₃-C₁₀ cycloalkyl, 3-10 membered heterocyclyl or 5-6 membered monocyclic heteroaryl, each of which is optionally substituted with one or more R^{6c};
X^{c} is CH or N;
Y^{c} is CHOH or NH;
R^{1c} is H or C₁-C₆ alkyl;
R^{2c} is C₁-C₆ alkyl, C₂-C₆ alkenyl, C₂-C₆ alkynyl, C₃-C₈ cycloalkyl, 3-8 membered heterocyclyl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl, C₁-C₆ alkoxy and 3-8 membered heterocyclyl;
R^{3c} is H, C₁-C₆ alkyl, C₂-C₆ alkenyl, C₂-C₆ alkynyl, C₃-C₈ cycloalkyl, 3- to 8-membered heterocyclyl or 5- or 6-membered heteroaryl, each of which is optionally substituted with one more substituent selected from halo, hydroxyl, cyano, aminyl, C₁-C₆ alkyl, C₂-C₆ alkenyl, C₂-C₆ alkynyl and C₁-C₆ alkoxy;
R^{4c} is a heteroaryl selected from oxazolyl, isoxazolyl, 1, 2, 3-oxadiazolyl, 1, 2, 4-oxadiazolyl, 1, 2, 5-oxadiazolyl, 1, 3, 4-oxadiazolyl, 1, 2, 3-triazolyl, 1, 2, 4-triazolyl, thiazolyl, isothiazolyl, 1, 2, 3-thiadiazolyl, 1, 2, 4-thiadiazolyl, 1, 2, 5-thiadiazolyl and 1, 3, 4-thiadiazolyl, each of which is optionally substituted with one more substituents selected from halo, C₁-C₆ alkyl, C₂-C₆ alkenyl, C₂-C₆ alkynyl, C₁-C₆ haloalkyl, C₃-C₈ cycloalkyl, cyano, aminyl, C₁-C₆ hydroxylalkyl, C₁-C₆ cyanoalkyl, 3- to 8-membered heterocyclyl, C₃-C₈ haloalkylcycloalkyl, C₃-C₈ aminylalkylcycloalkyl, C₃-C₈ alkylcycloalkyl, 3- to 8-membered heterocyclylalkyl, 3- to 8-membered alkylheterocyclylcycloalkyl, 3- to 8-membered haloheterocyclylalkyl, and C₃-C₈ halocycloalkyl;
R^{5c} is H, C₁-C₆ alkyl, C₂-C₆ alkenyl, C₂-C₆ alkynyl, C₃-C₈ cycloalkyl, 3- to 8-membered heterocyclyl, C₆-C₁₀ aryl or 5- or 6-membered heteroaryl, each of which is optionally substituted with one more substituents selected from halo, hydroxyl, cyano, aminyl, C₁-C₆ alkyl, C₂-C₆ alkenyl, C₂-C₆ alkynyl and C₁-C₆ alkoxy; and
R^{6c} is, at each occurrence, independently halo, C₁-C₆ alkyl, C₁-C₆ alkoxy, cyano, C₁-C₆ hydroxylalkyl or C₁-C₆ haloalkyl.

109. (canceled)

110. The method of claim 1, wherein the NEK7 small molecule inhibitor compound has the following Structure (Id): 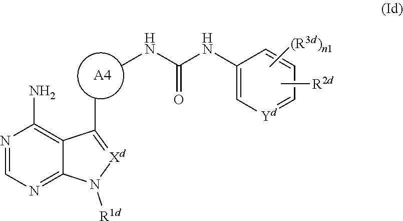
or a pharmaceutically acceptable salt, stereoisomer, or prodrug thereof, wherein:
A4 is C₆-C₁₀ aryl, C₃-C₁₀ cycloalkyl, 3-10 membered heterocyclyl, or 5-6 membered heteroaryl;
X^{d} is N or CR^{4d};
Y^{d} is N or CH;
R^{1d} is C₁-C₆ alkyl, C₁-C₆ hydroxylalkyl, C₃-C₁₀ cycloalkyl, or 3-10 membered heterocyclyl;
R^{2d} is a 3-10 membered heterocyclyl, 3-10 membered heterocyclylalkyl, 3-10 membered heterocyclylalkenyl, 3-10 membered heterocyclylcarbonyl, 3-10 membered heterocyclyloxy, or 5-6 membered heteroaryl;
R^{3d} is, at each occurrence, independently halo, cyano, C₁-C₆ alkyl, C₁-C₆ haloalkyl, C₁-C₆ alkoxy, C₁-C₆ haloalkoxy, or C₃-C₈ cycloalkyl;
R^{4d} is H, C₁-C₆ alkyl, C₁-C₆ haloalkyl, or C₃-C₈ cycloalkyl; and
n1 is 0, 1, 2, 3, or 4.

111. (canceled)

112. The method of claim 1, wherein the NEK7 small molecule inhibitor compound has the following Structure (Ie): 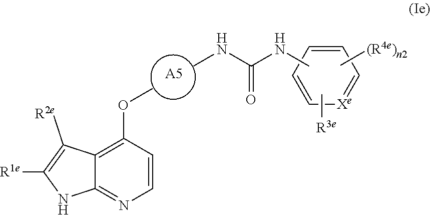
or a pharmaceutically acceptable salt, stereoisomer, or prodrug thereof, wherein:
X^{e} is N or CH;
A5 is C₆-C₁₀ arylene, C₃-C₁₀ cycloalkylene, 3-10 membered heterocyclylene, or 5-6 membered heteroarylene;
R^{1e} is H, halo, C₁-C₆ alkyl, C₃-C₁₀ cycloalkyl, or 3-10 membered heterocyclyl;
R^{2e} is H, halo, C₁-C₆ alkyl, C₁-C₆ haloalkyl, C₃-C₁₀ cycloalkyl, or 3-10 membered heterocyclyl;
R^{3e} is aminylalkyl, 3-10 membered heterocyclyl, 3-10 membered heterocyclylalkyl, 3-10 membered heterocyclylcarbonyl, 3-10 membered heterocyclylalkenyl, 3-10 membered N-heterocyclyloxy, or 5-6 membered heteroaryl;
R^{4e} is, at each occurrence, independently halo, cyano, C₁-C₆ alkyl, C₁-C₆ haloalkyl, C₁-C₆ alkoxy, C₁-C₆ haloalkoxy, C₃-C₈ halocycloalkyl, or C₃-C₈ cycloalkyl; and
n2 is 0, 1, 2, 3, or 4.

113. (canceled)

114. The method of claim 1, wherein the NEK7 small molecule inhibitor compound has the following Structure (If): 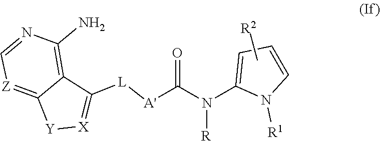
or a pharmaceutically acceptable salt, stereoisomer, or prodrug thereof, wherein:
A′ is C═O, C(R)₂ or NR;
L is a divalent group selected from C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
X is CR or N;
Y is NR or S;
Z is CR or N;
R¹ is C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
R² is —R, halogen, -haloalkyl, —OR, —SR, —CN, —NO₂, —SO₂R, —SOR, —C(═O)R, —CO₂R,
—C(═O)N(R)₂, —NRC(═O)R, —NRC(═O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 hetero atoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

115. The method of claim 4441, wherein the NEK7 small molecule inhibitor compound has one of the following structures, or a pharmaceutically acceptable salt, stereoisomer, or prodrug thereof:

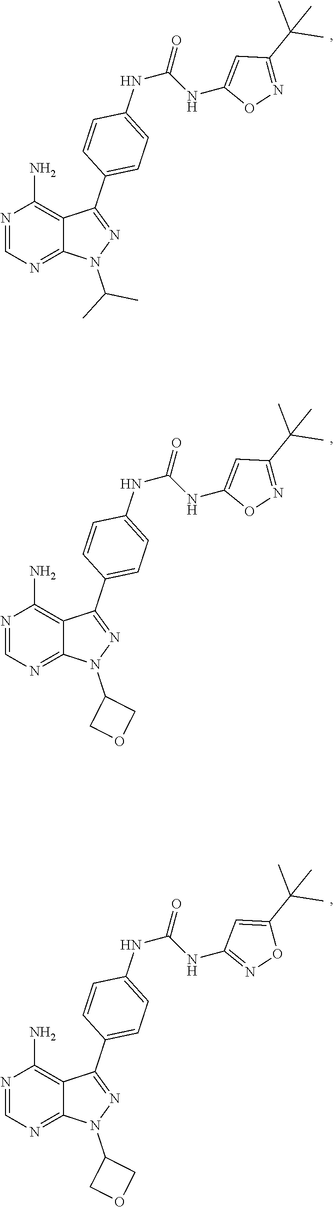 [SMILES: CC(C)n1nc(-c2ccc(NC(=O)Nc3cc(C(C)(C)C)no3)cc2)c2c(N)ncnc21 ; CC(C)(C)c1cc(NC(=O)Nc2ccc(-c3nn(C4COC4)c4ncnc(N)c34)cc2)on1 ; CC(C)(C)c1cc(NC(=O)Nc2ccc(-c3nn(C4COC4)c4ncnc(N)c34)cc2)no1]

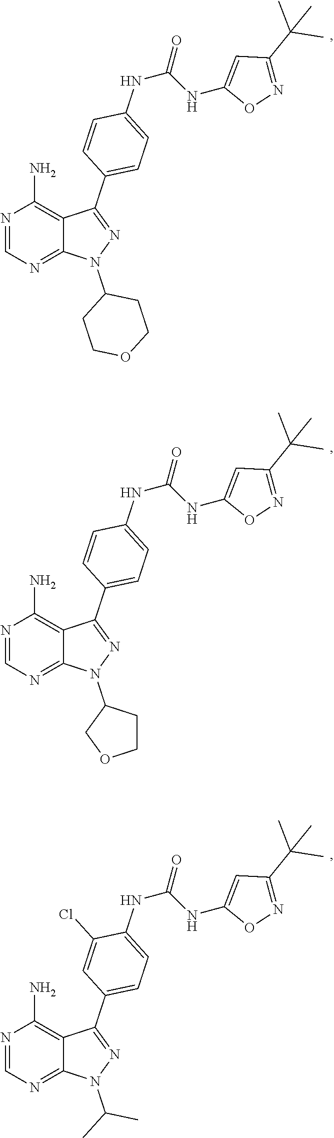 [SMILES: CC(C)(C)c1cc(NC(=O)Nc2ccc(-c3nn(C4CCOCC4)c4ncnc(N)c34)cc2)on1 ; CC(C)(C)c1cc(NC(=O)Nc2ccc(-c3nn(C4CCOC4)c4ncnc(N)c34)cc2)on1 ; CC(C)n1nc(-c2ccc(NC(=O)Nc3cc(C(C)(C)C)no3)c(Cl)c2)c2c(N)ncnc21]

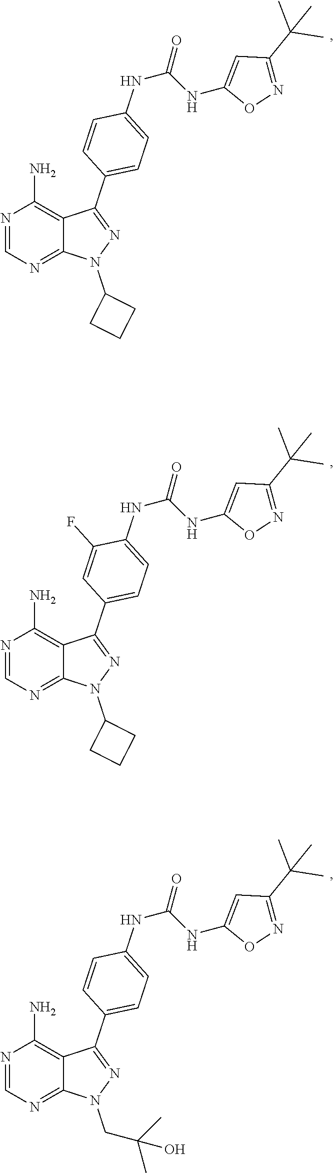 [SMILES: CC(C)(C)c1cc(NC(=O)Nc2ccc(-c3nn(C4CCC4)c4ncnc(N)c34)cc2)on1 ; CC(C)(C)c1cc(NC(=O)Nc2ccc(-c3nn(C4CCC4)c4ncnc(N)c34)cc2F)on1 ; CC(C)(O)Cn1nc(-c2ccc(NC(=O)Nc3cc(C(C)(C)C)no3)cc2)c2c(N)ncnc21]

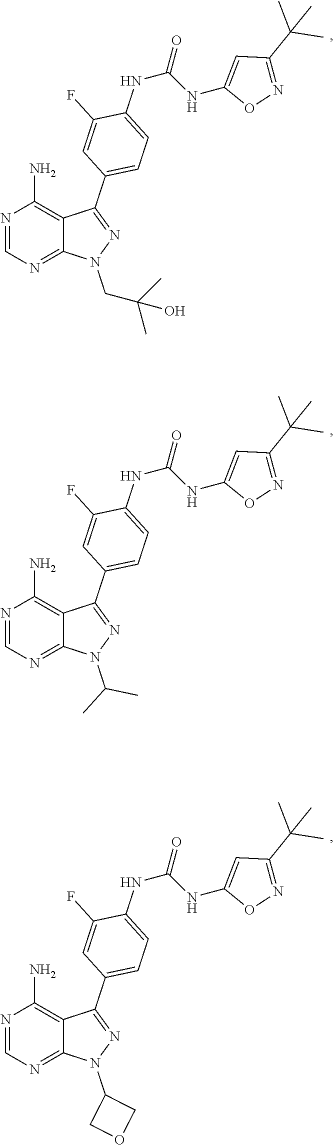 [SMILES: CC(C)(O)Cn1nc(-c2ccc(NC(=O)Nc3cc(C(C)(C)C)no3)c(F)c2)c2c(N)ncnc21 ; CC(C)n1nc(-c2ccc(NC(=O)Nc3cc(C(C)(C)C)no3)c(F)c2)c2c(N)ncnc21 ; CC(C)(C)c1cc(NC(=O)Nc2ccc(-c3nn(C4COC4)c4ncnc(N)c34)cc2F)on1]

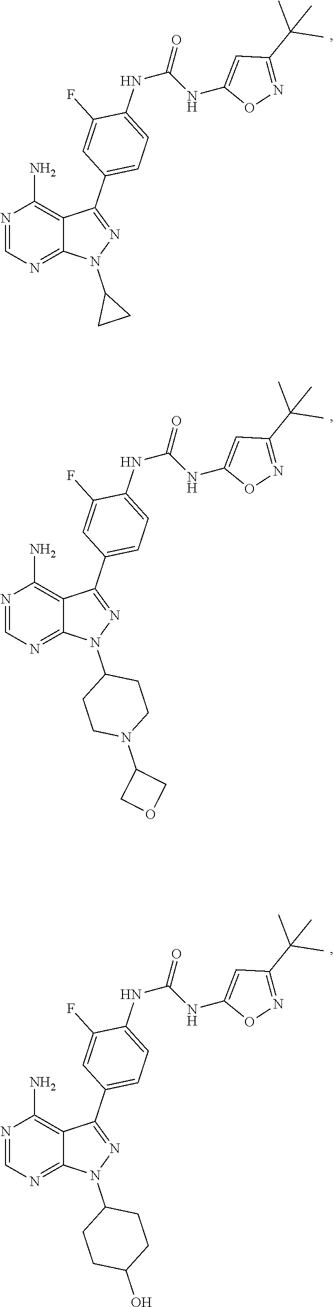 [SMILES: CC(C)(C)c1cc(NC(=O)Nc2ccc(-c3nn(C4CC4)c4ncnc(N)c34)cc2F)on1 ; CC(C)(C)c1cc(NC(=O)Nc2ccc(-c3nn(C4CCN(C5COC5)CC4)c4ncnc(N)c34)cc2F)on1 ; CC(C)(C)c1cc(NC(=O)Nc2ccc(-c3nn(C4CCC(O)CC4)c4ncnc(N)c34)cc2F)on1]

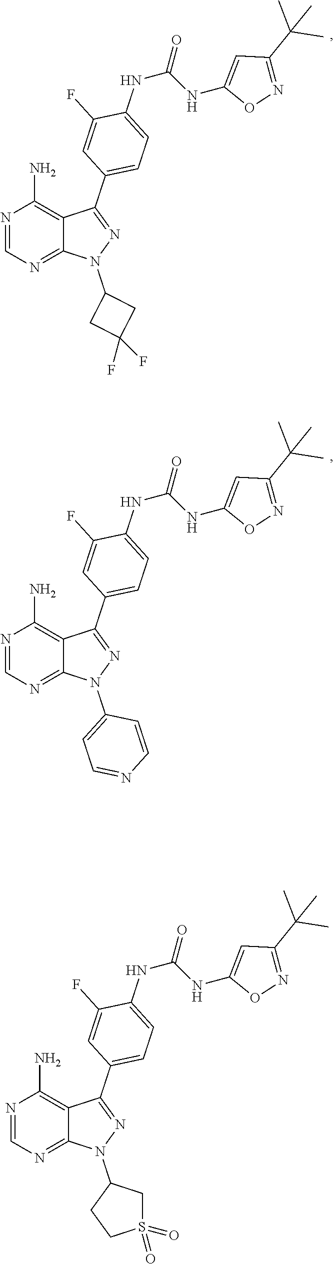 [SMILES: CC(C)(C)c1cc(NC(=O)Nc2ccc(-c3nn(C4CC(F)(F)C4)c4ncnc(N)c34)cc2F)on1 ; CC(C)(C)c1cc(NC(=O)Nc2ccc(-c3nn(-c4ccncc4)c4ncnc(N)c34)cc2F)on1 ; CC(C)(C)c1cc(NC(=O)Nc2ccc(-c3nn(C4CCS(=O)(=O)C4)c4ncnc(N)c34)cc2F)on1]

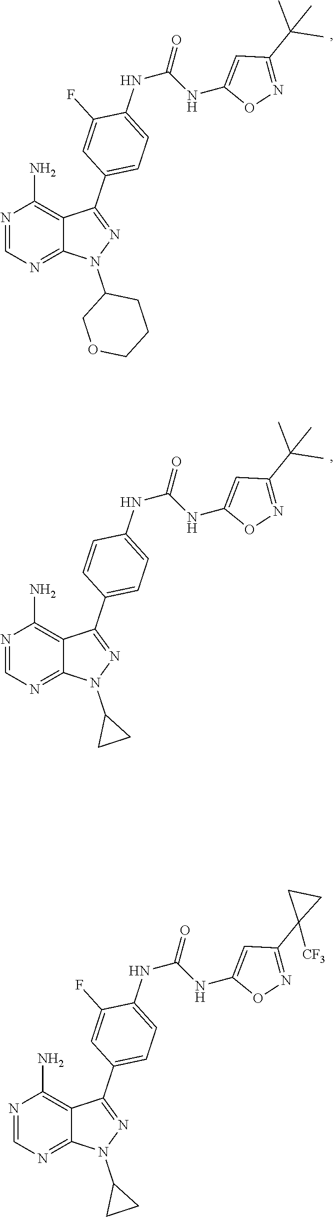 [SMILES: CC(C)(C)c1cc(NC(=O)Nc2ccc(-c3nn(C4CCCOC4)c4ncnc(N)c34)cc2F)on1 ; CC(C)(C)c1cc(NC(=O)Nc2ccc(-c3nn(C4CC4)c4ncnc(N)c34)cc2)on1 ; Nc1ncnc2c1c(-c1ccc(NC(=O)Nc3cc(C4(C(F)(F)F)CC4)no3)c(F)c1)nn2C1CC1]

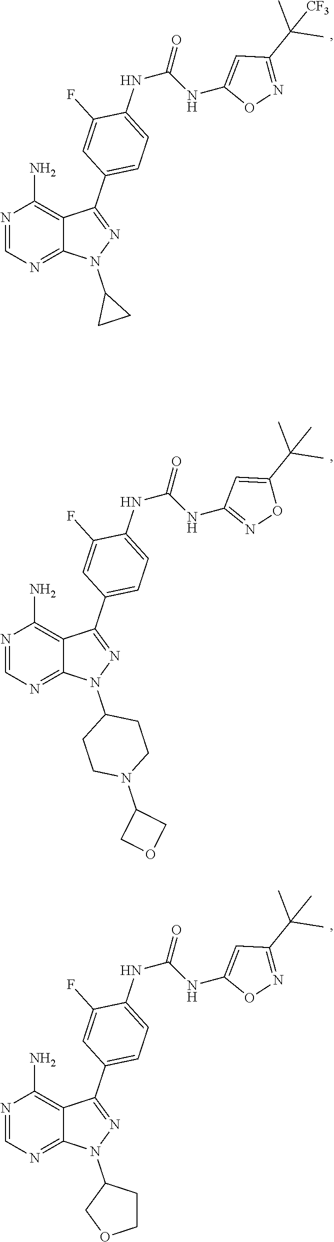 [SMILES: CC(C)(c1cc(NC(=O)Nc2ccc(-c3nn(C4CC4)c4ncnc(N)c34)cc2F)on1)C(F)(F)F ; CC(C)(C)c1cc(NC(=O)Nc2ccc(-c3nn(C4CCN(C5COC5)CC4)c4ncnc(N)c34)cc2F)no1 ; CC(C)(C)c1cc(NC(=O)Nc2ccc(-c3nn(C4CCOC4)c4ncnc(N)c34)cc2F)on1]

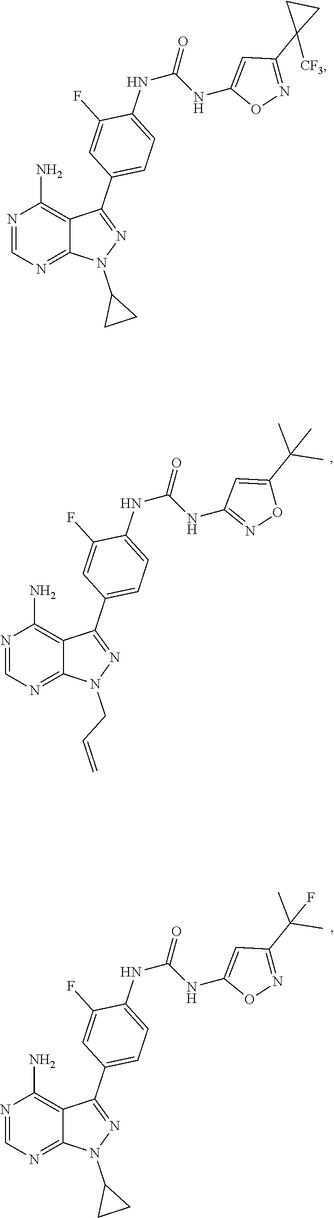 [SMILES: Nc1ncnc2c1c(-c1ccc(NC(=O)Nc3cc(C4(C(F)(F)F)CC4)no3)c(F)c1)nn2C1CC1 ; C=CCn1nc(-c2ccc(NC(=O)Nc3cc(C(C)(C)C)on3)c(F)c2)c2c(N)ncnc21 ; CC(C)(F)c1cc(NC(=O)Nc2ccc(-c3nn(C4CC4)c4ncnc(N)c34)cc2F)on1]

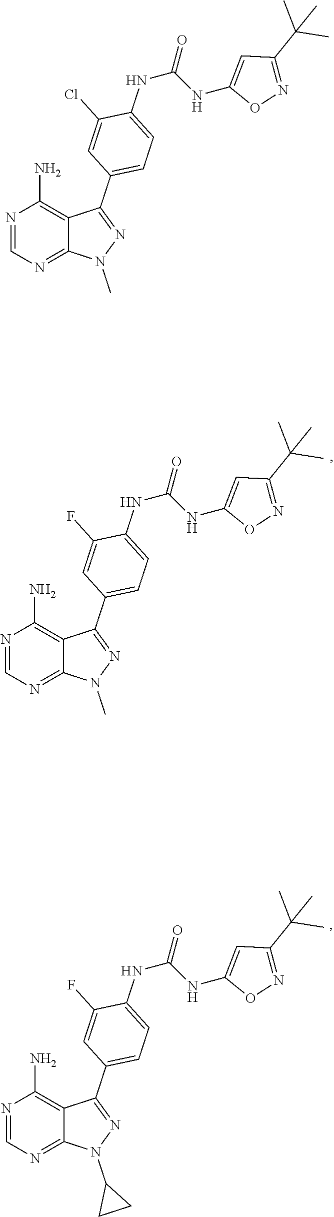 [SMILES: Cn1nc(-c2ccc(NC(=O)Nc3cc(C(C)(C)C)no3)c(Cl)c2)c2c(N)ncnc21 ; Cn1nc(-c2ccc(NC(=O)Nc3cc(C(C)(C)C)no3)c(F)c2)c2c(N)ncnc21 ; CC(C)(C)c1cc(NC(=O)Nc2ccc(-c3nn(C4CC4)c4ncnc(N)c34)cc2F)on1]

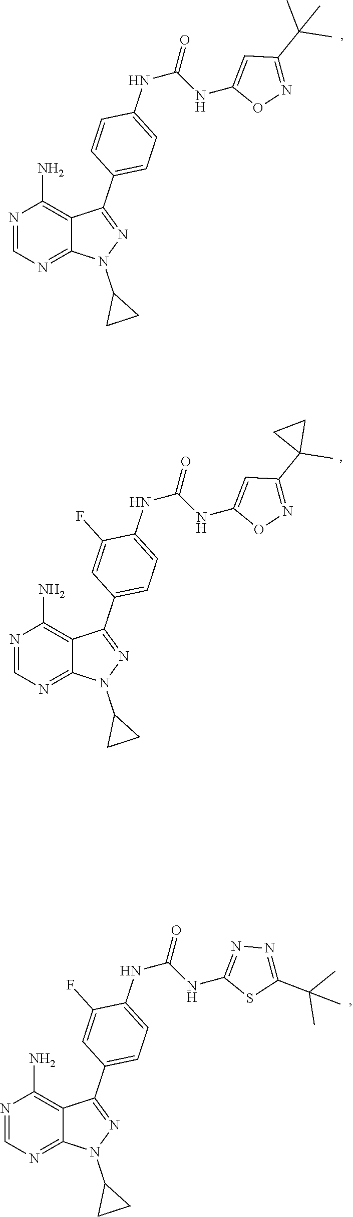 [SMILES: CC(C)(C)c1cc(NC(=O)Nc2ccc(-c3nn(C4CC4)c4ncnc(N)c34)cc2)on1 ; CC1(c2cc(NC(=O)Nc3ccc(-c4nn(C5CC5)c5ncnc(N)c45)cc3F)on2)CC1 ; CC(C)(C)c1nnc(NC(=O)Nc2ccc(-c3nn(C4CC4)c4ncnc(N)c34)cc2F)s1]

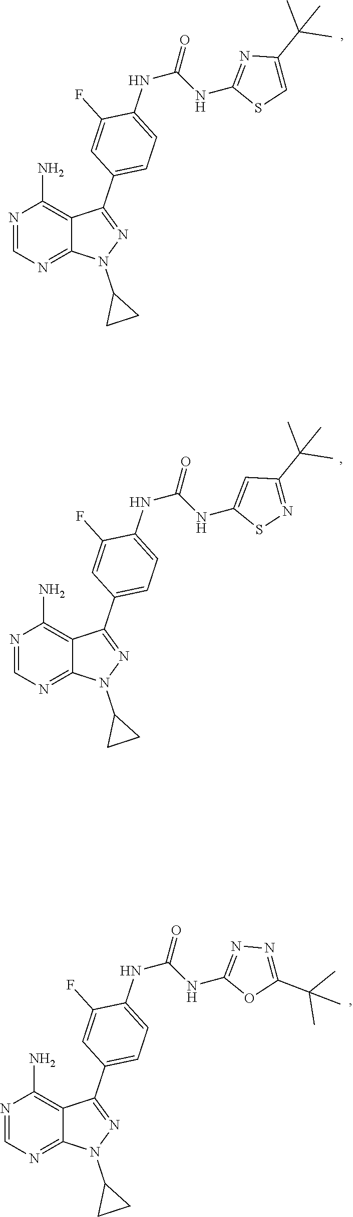 [SMILES: CC(C)(C)c1csc(NC(=O)Nc2ccc(-c3nn(C4CC4)c4ncnc(N)c34)cc2F)n1 ; CC(C)(C)c1cc(NC(=O)Nc2ccc(-c3nn(C4CC4)c4ncnc(N)c34)cc2F)sn1 ; CC(C)(C)c1nnc(NC(=O)Nc2ccc(-c3nn(C4CC4)c4ncnc(N)c34)cc2F)o1]

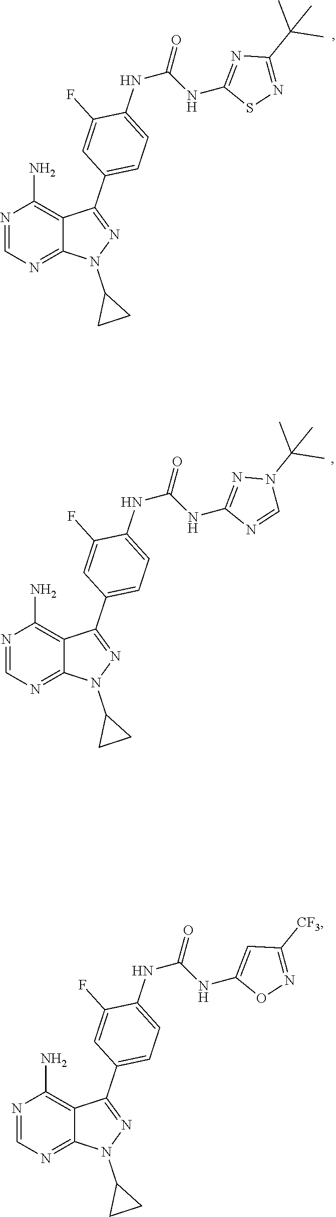 [SMILES: CC(C)(C)c1nsc(NC(=O)Nc2ccc(-c3nn(C4CC4)c4ncnc(N)c34)cc2F)n1 ; CC(C)(C)n1cnc(NC(=O)Nc2ccc(-c3nn(C4CC4)c4ncnc(N)c34)cc2F)n1 ; Nc1ncnc2c1c(-c1ccc(NC(=O)Nc3cc(C(F)(F)F)no3)c(F)c1)nn2C1CC1]

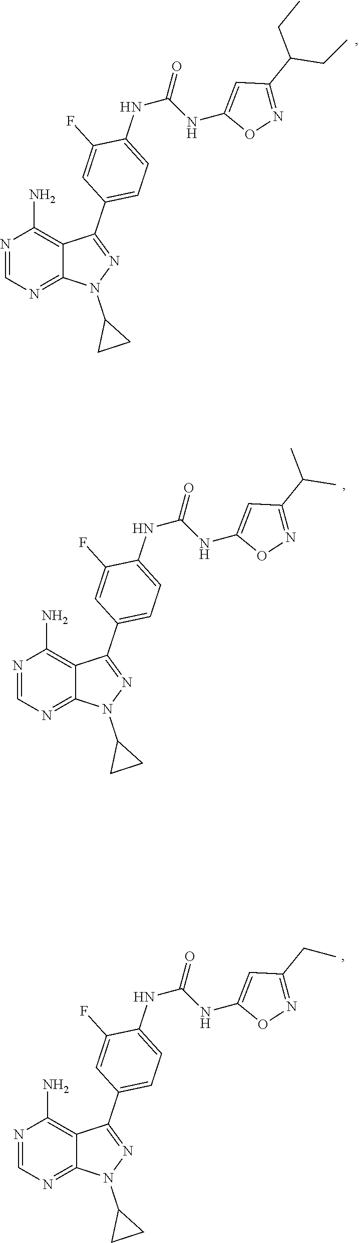 [SMILES: CCC(CC)c1cc(NC(=O)Nc2ccc(-c3nn(C4CC4)c4ncnc(N)c34)cc2F)on1 ; CC(C)c1cc(NC(=O)Nc2ccc(-c3nn(C4CC4)c4ncnc(N)c34)cc2F)on1 ; CCc1cc(NC(=O)Nc2ccc(-c3nn(C4CC4)c4ncnc(N)c34)cc2F)on1]

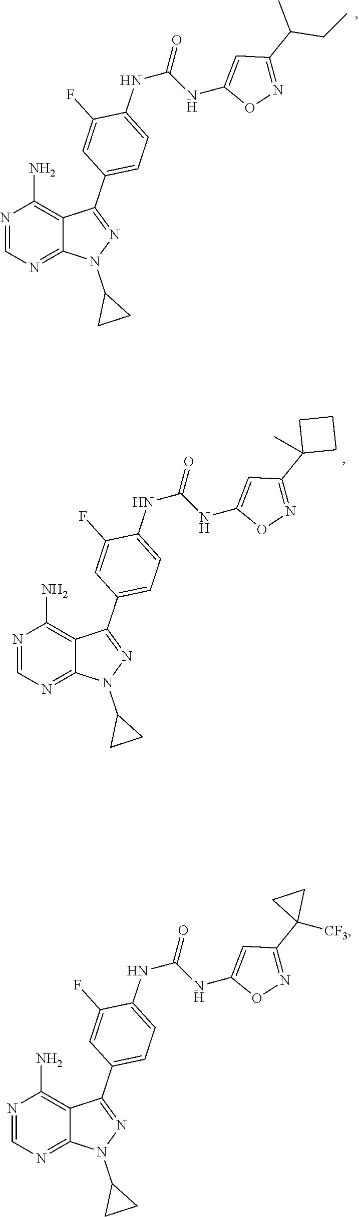 [SMILES: CCC(C)c1cc(NC(=O)Nc2ccc(-c3nn(C4CC4)c4ncnc(N)c34)cc2F)on1 ; CC1(c2cc(NC(=O)Nc3ccc(-c4nn(C5CC5)c5ncnc(N)c45)cc3F)on2)CCC1 ; Nc1ncnc2c1c(-c1ccc(NC(=O)Nc3cc(C4(C(F)(F)F)CC4)no3)c(F)c1)nn2C1CC1]

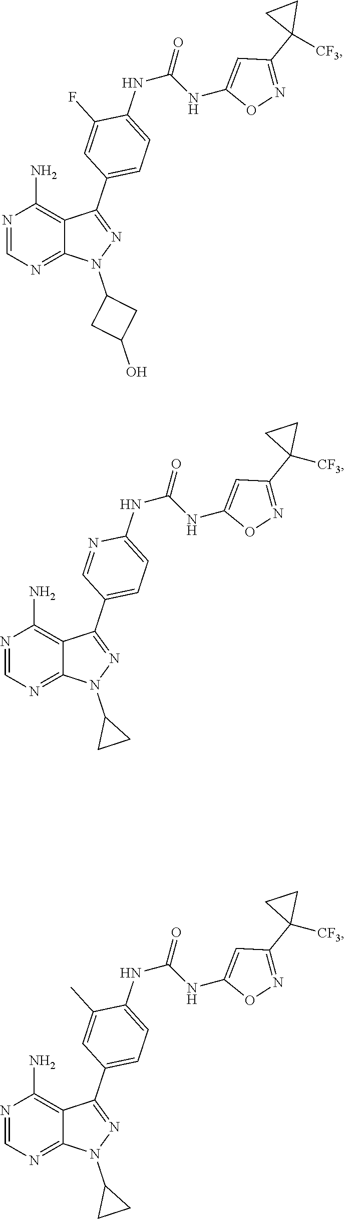 [SMILES: Nc1ncnc2c1c(-c1ccc(NC(=O)Nc3cc(C4(C(F)(F)F)CC4)no3)c(F)c1)nn2C1CC(O)C1 ; Nc1ncnc2c1c(-c1ccc(NC(=O)Nc3cc(C4(C(F)(F)F)CC4)no3)nc1)nn2C1CC1 ; Cc1cc(-c2nn(C3CC3)c3ncnc(N)c23)ccc1NC(=O)Nc1cc(C2(C(F)(F)F)CC2)no1]

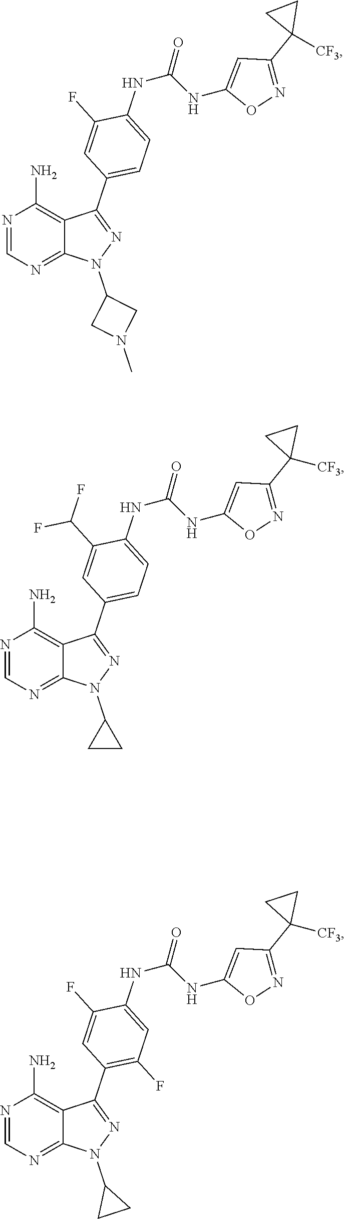 [SMILES: CN1CC(n2nc(-c3ccc(NC(=O)Nc4cc(C5(C(F)(F)F)CC5)no4)c(F)c3)c3c(N)ncnc32)C1 ; Nc1ncnc2c1c(-c1ccc(NC(=O)Nc3cc(C4(C(F)(F)F)CC4)no3)c(C(F)F)c1)nn2C1CC1 ; Nc1ncnc2c1c(-c1cc(F)c(NC(=O)Nc3cc(C4(C(F)(F)F)CC4)no3)cc1F)nn2C1CC1]

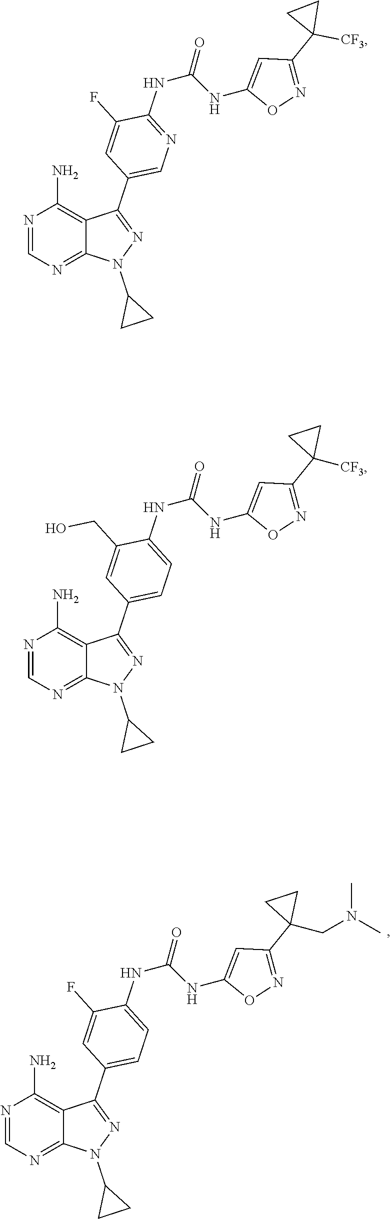 [SMILES: Nc1ncnc2c1c(-c1cnc(NC(=O)Nc3cc(C4(C(F)(F)F)CC4)no3)c(F)c1)nn2C1CC1 ; Nc1ncnc2c1c(-c1ccc(NC(=O)Nc3cc(C4(C(F)(F)F)CC4)no3)c(CO)c1)nn2C1CC1 ; CN(C)CC1(c2cc(NC(=O)Nc3ccc(-c4nn(C5CC5)c5ncnc(N)c45)cc3F)on2)CC1]

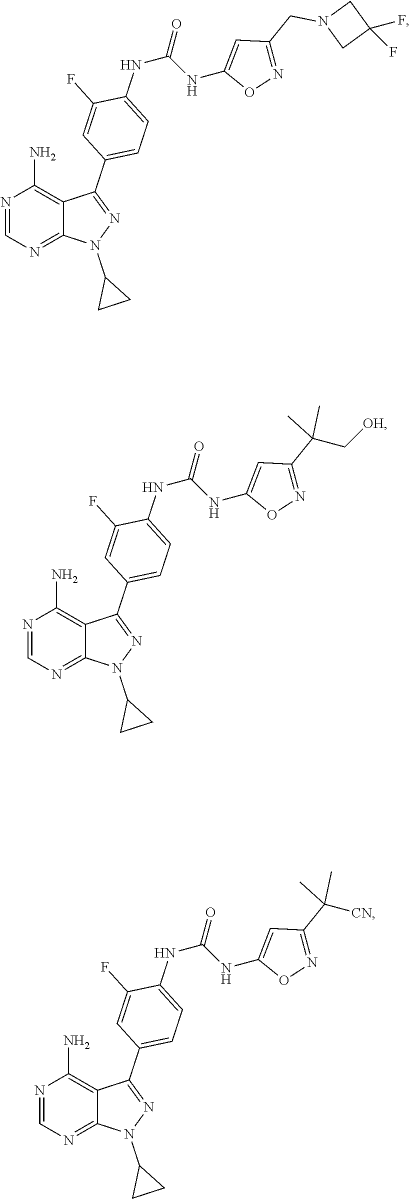 [SMILES: Nc1ncnc2c1c(-c1ccc(NC(=O)Nc3cc(CN4CC(F)(F)C4)no3)c(F)c1)nn2C1CC1 ; CC(C)(CO)c1cc(NC(=O)Nc2ccc(-c3nn(C4CC4)c4ncnc(N)c34)cc2F)on1 ; CC(C)(C#N)c1cc(NC(=O)Nc2ccc(-c3nn(C4CC4)c4ncnc(N)c34)cc2F)on1]

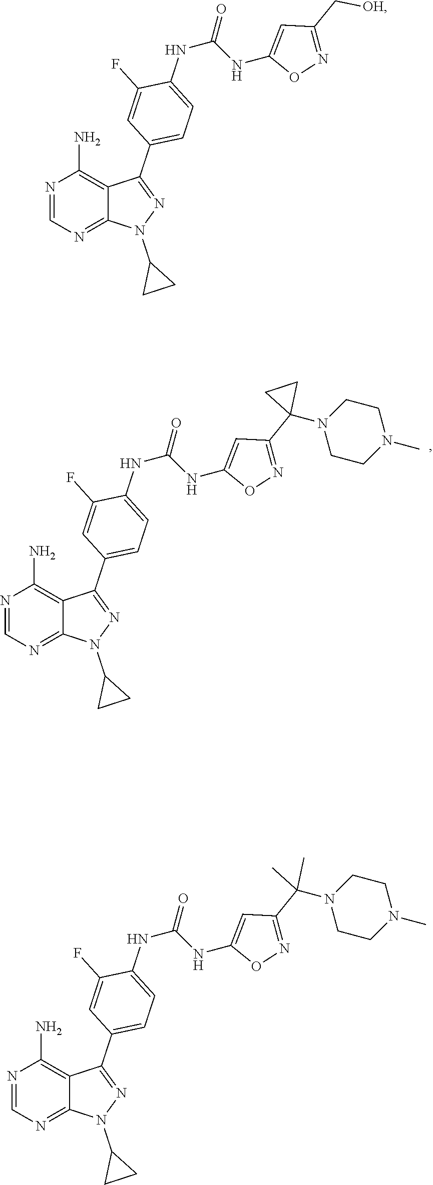 [SMILES: Nc1ncnc2c1c(-c1ccc(NC(=O)Nc3cc(CO)no3)c(F)c1)nn2C1CC1 ; CN1CCN(C2(c3cc(NC(=O)Nc4ccc(-c5nn(C6CC6)c6ncnc(N)c56)cc4F)on3)CC2)CC1 ; CN1CCN(C(C)(C)c2cc(NC(=O)Nc3ccc(-c4nn(C5CC5)c5ncnc(N)c45)cc3F)on2)CC1]

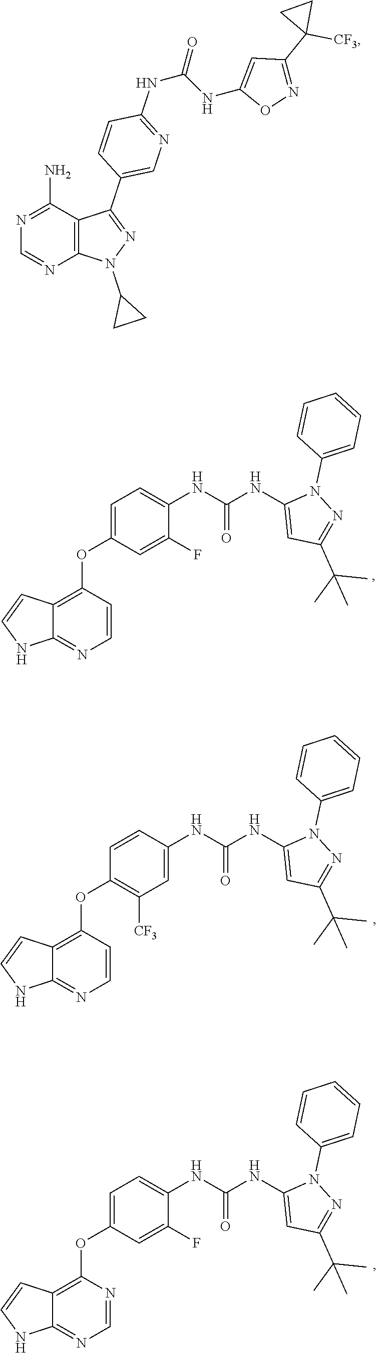 [SMILES: Nc1ncnc2c1c(-c1ccc(NC(=O)Nc3cc(C4(C(F)(F)F)CC4)no3)nc1)nn2C1CC1 ; CC(C)(C)c1cc(NC(=O)Nc2ccc(Oc3ccnc4[nH]ccc34)cc2F)n(-c2ccccc2)n1 ; CC(C)(C)c1cc(NC(=O)Nc2ccc(Oc3ccnc4[nH]ccc34)c(C(F)(F)F)c2)n(-c2ccccc2)n1 ; CC(C)(C)c1cc(NC(=O)Nc2ccc(Oc3ncnc4[nH]ccc34)cc2F)n(-c2ccccc2)n1]

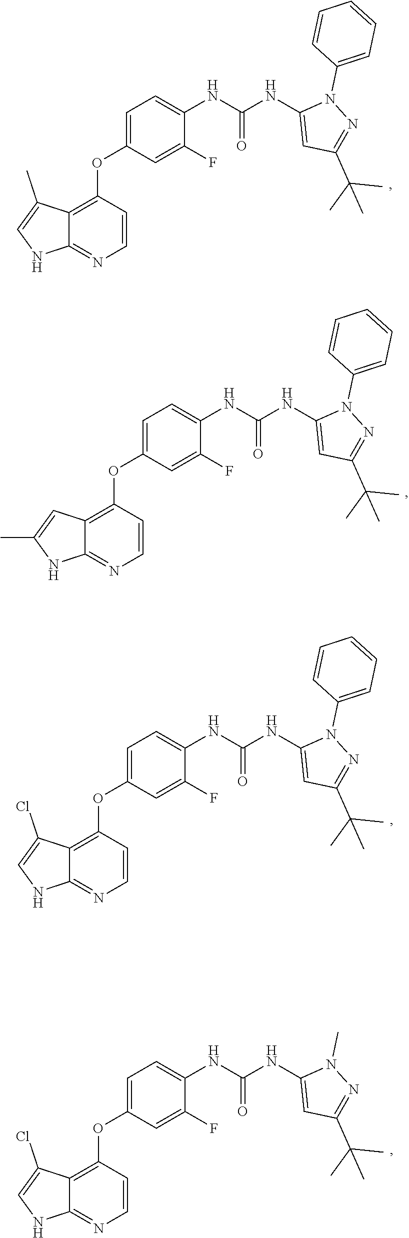

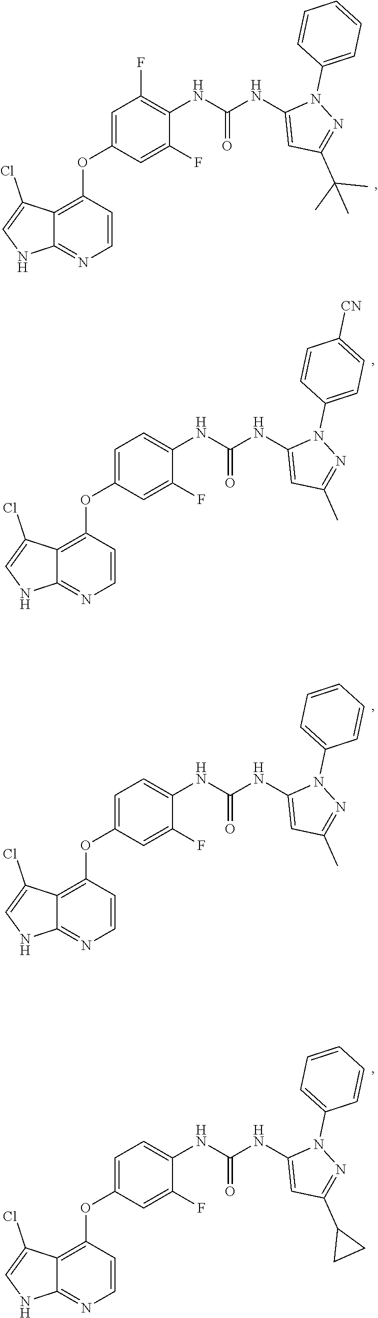 [SMILES: CC(C)(C)c1cc(NC(=O)Nc2c(F)cc(Oc3ccnc4[nH]cc(Cl)c34)cc2F)n(-c2ccccc2)n1 ; Cc1cc(NC(=O)Nc2ccc(Oc3ccnc4[nH]cc(Cl)c34)cc2F)n(-c2ccc(C#N)cc2)n1 ; Cc1cc(NC(=O)Nc2ccc(Oc3ccnc4[nH]cc(Cl)c34)cc2F)n(-c2ccccc2)n1 ; O=C(Nc1ccc(Oc2ccnc3[nH]cc(Cl)c23)cc1F)Nc1cc(C2CC2)nn1-c1ccccc1]

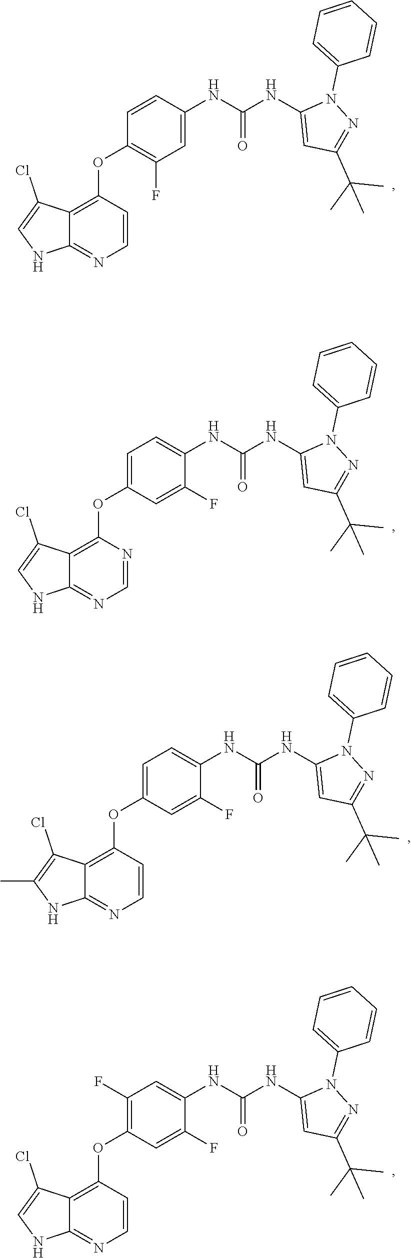

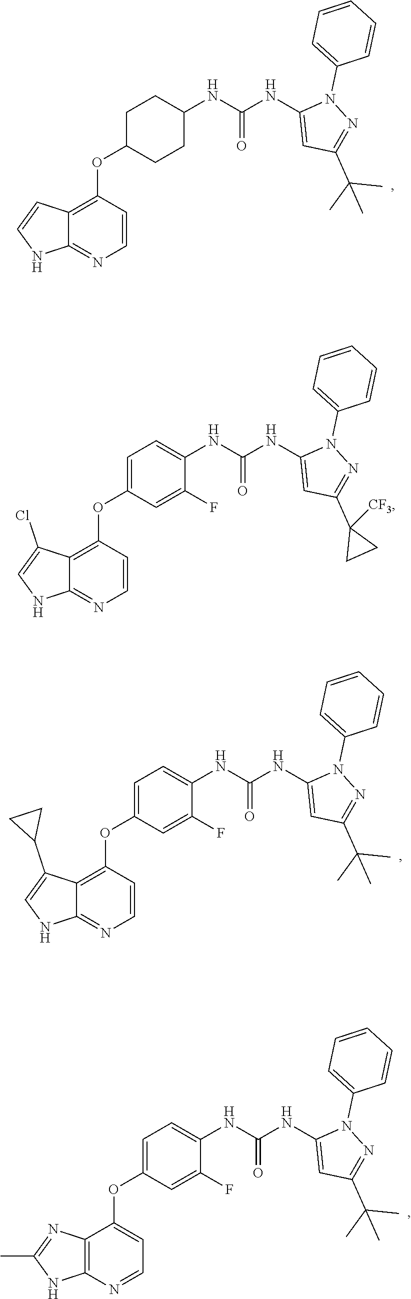

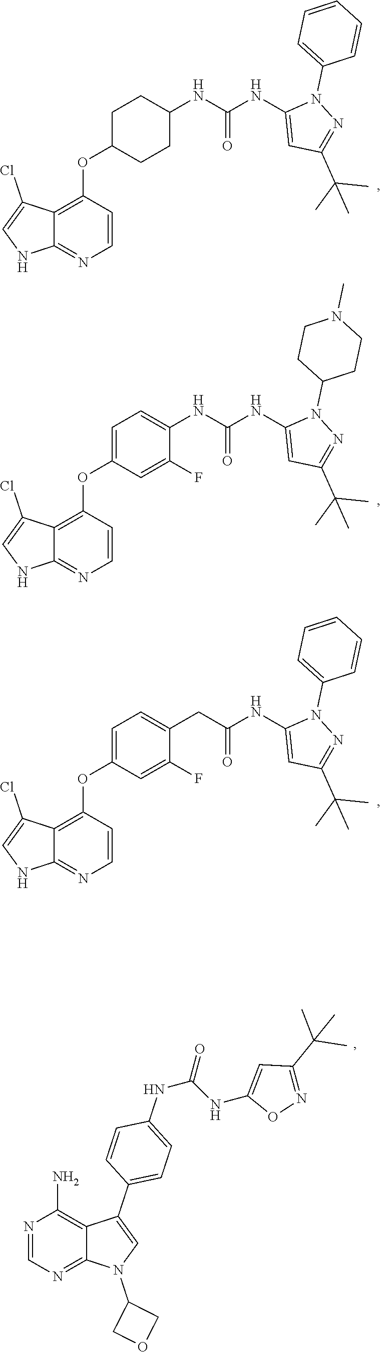 [SMILES: CC(C)(C)c1cc(NC(=O)NC2CCC(Oc3ccnc4[nH]cc(Cl)c34)CC2)n(-c2ccccc2)n1 ; CN1CCC(n2nc(C(C)(C)C)cc2NC(=O)Nc2ccc(Oc3ccnc4[nH]cc(Cl)c34)cc2F)CC1 ; CC(C)(C)c1cc(NC(=O)Cc2ccc(Oc3ccnc4[nH]cc(Cl)c34)cc2F)n(-c2ccccc2)n1 ; CC(C)(C)c1cc(NC(=O)Nc2ccc(-c3cn(C4COC4)c4ncnc(N)c34)cc2)on1]

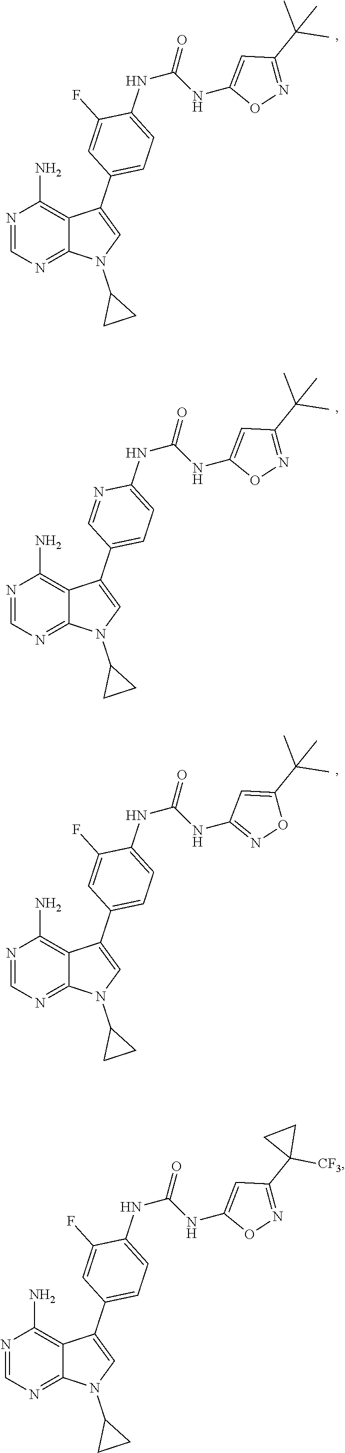 [SMILES: CC(C)(C)c1cc(NC(=O)Nc2ccc(-c3cn(C4CC4)c4ncnc(N)c34)cc2F)on1 ; CC(C)(C)c1cc(NC(=O)Nc2ccc(-c3cn(C4CC4)c4ncnc(N)c34)cn2)on1 ; CC(C)(C)c1cc(NC(=O)Nc2ccc(-c3cn(C4CC4)c4ncnc(N)c34)cc2F)no1 ; Nc1ncnc2c1c(-c1ccc(NC(=O)Nc3cc(C4(C(F)(F)F)CC4)no3)c(F)c1)cn2C1CC1]

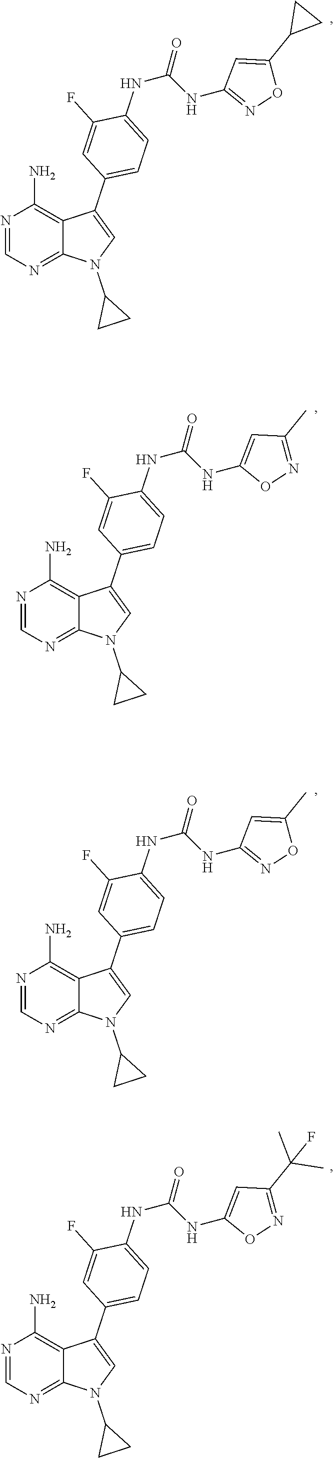 [SMILES: Nc1ncnc2c1c(-c1ccc(NC(=O)Nc3cc(C4CC4)on3)c(F)c1)cn2C1CC1 ; Cc1cc(NC(=O)Nc2ccc(-c3cn(C4CC4)c4ncnc(N)c34)cc2F)on1 ; Cc1cc(NC(=O)Nc2ccc(-c3cn(C4CC4)c4ncnc(N)c34)cc2F)no1 ; CC(C)(F)c1cc(NC(=O)Nc2ccc(-c3cn(C4CC4)c4ncnc(N)c34)cc2F)on1]

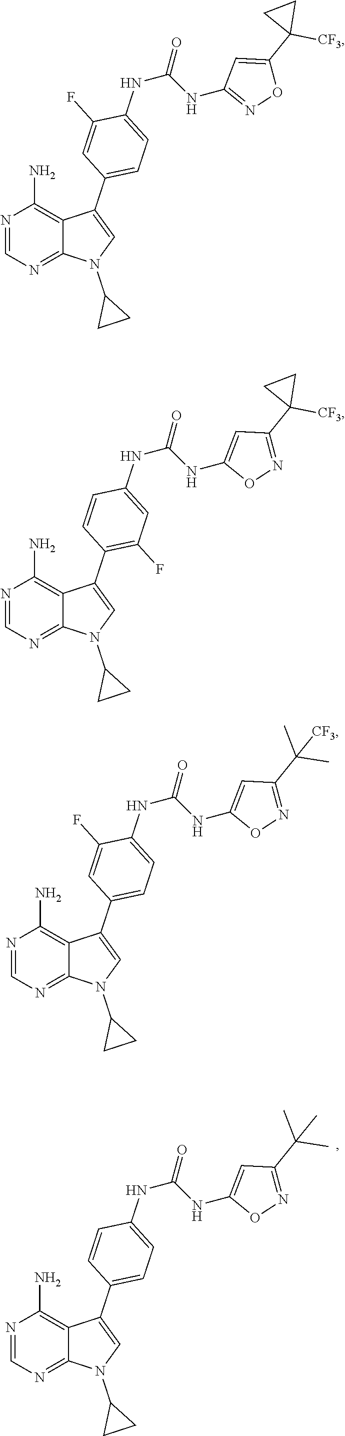 [SMILES: Nc1ncnc2c1c(-c1ccc(NC(=O)Nc3cc(C4(C(F)(F)F)CC4)on3)c(F)c1)cn2C1CC1 ; Nc1ncnc2c1c(-c1ccc(NC(=O)Nc3cc(C4(C(F)(F)F)CC4)no3)cc1F)cn2C1CC1 ; CC(C)(c1cc(NC(=O)Nc2ccc(-c3cn(C4CC4)c4ncnc(N)c34)cc2F)on1)C(F)(F)F ; CC(C)(C)c1cc(NC(=O)Nc2ccc(-c3cn(C4CC4)c4ncnc(N)c34)cc2)on1]

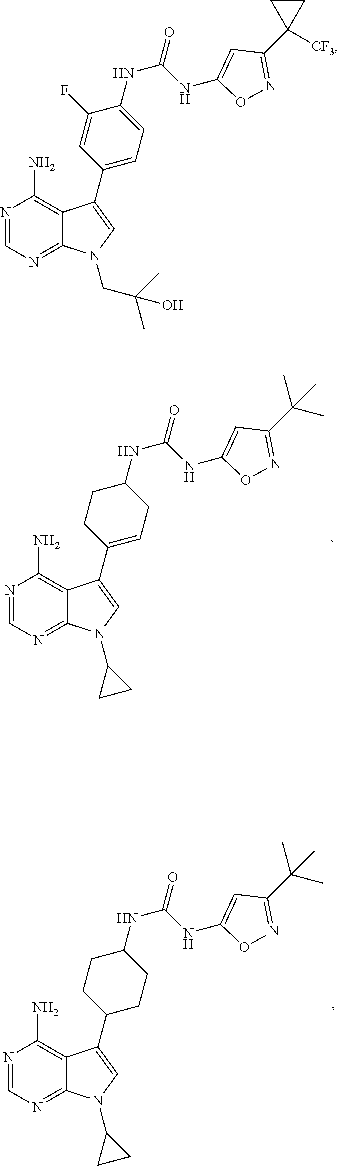 [SMILES: CC(C)(O)Cn1cc(-c2ccc(NC(=O)Nc3cc(C4(C(F)(F)F)CC4)no3)c(F)c2)c2c(N)ncnc21 ; CC(C)(C)c1cc(NC(=O)NC2CC=C(c3cn(C4CC4)c4ncnc(N)c34)CC2)on1 ; CC(C)(C)c1cc(NC(=O)NC2CCC(c3cn(C4CC4)c4ncnc(N)c34)CC2)on1]

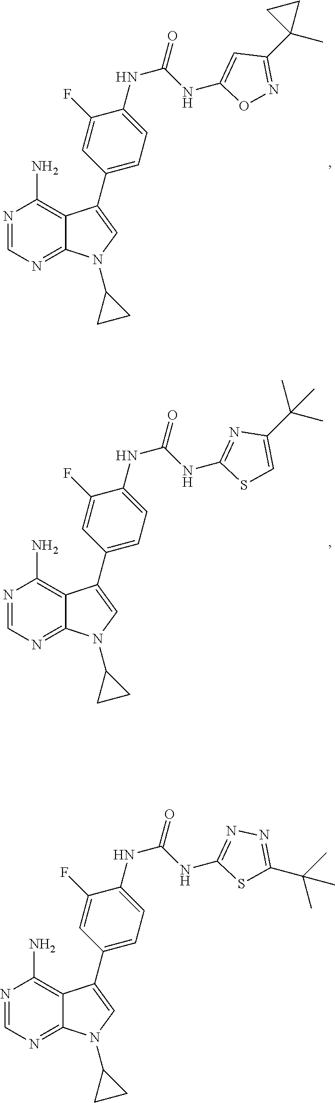 [SMILES: CC1(c2cc(NC(=O)Nc3ccc(-c4cn(C5CC5)c5ncnc(N)c45)cc3F)on2)CC1 ; CC(C)(C)c1csc(NC(=O)Nc2ccc(-c3cn(C4CC4)c4ncnc(N)c34)cc2F)n1 ; CC(C)(C)c1nnc(NC(=O)Nc2ccc(-c3cn(C4CC4)c4ncnc(N)c34)cc2F)s1]

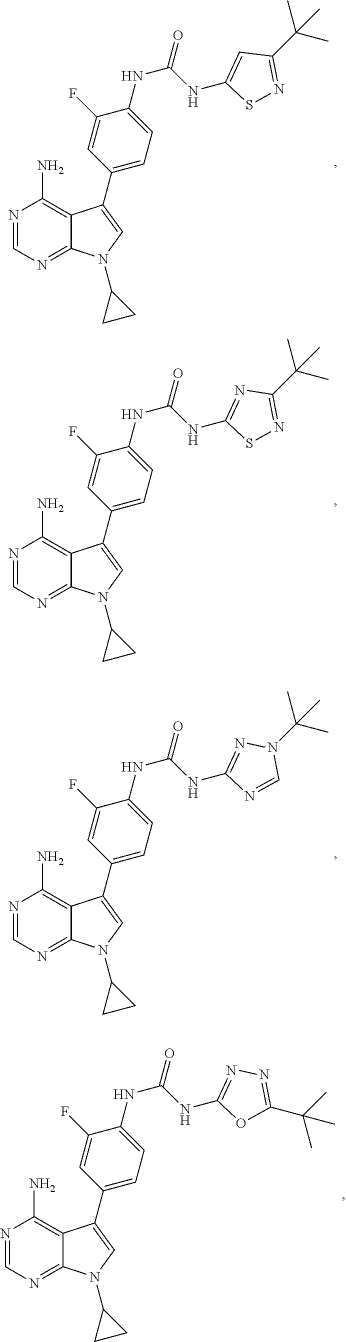 [SMILES: CC(C)(C)c1cc(NC(=O)Nc2ccc(-c3cn(C4CC4)c4ncnc(N)c34)cc2F)sn1 ; CC(C)(C)c1nsc(NC(=O)Nc2ccc(-c3cn(C4CC4)c4ncnc(N)c34)cc2F)n1 ; CC(C)(C)n1cnc(NC(=O)Nc2ccc(-c3cn(C4CC4)c4ncnc(N)c34)cc2F)n1 ; CC(C)(C)c1nnc(NC(=O)Nc2ccc(-c3cn(C4CC4)c4ncnc(N)c34)cc2F)o1]

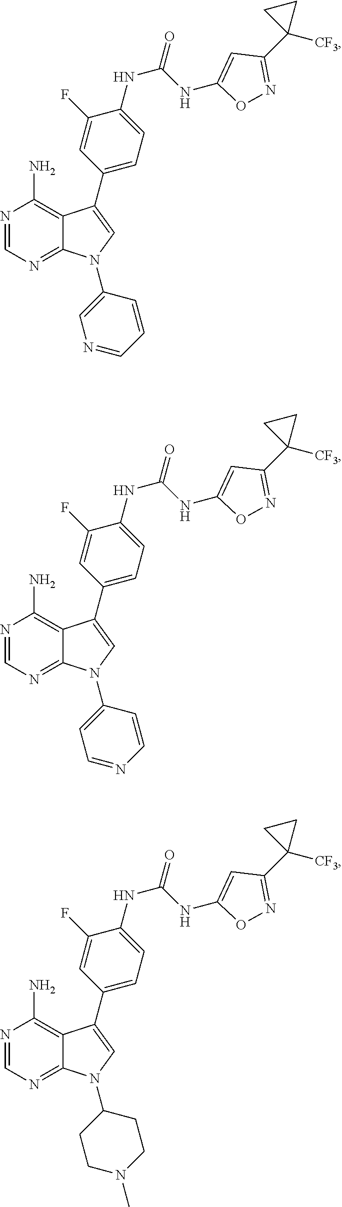 [SMILES: Nc1ncnc2c1c(-c1ccc(NC(=O)Nc3cc(C4(C(F)(F)F)CC4)no3)c(F)c1)cn2-c1cccnc1 ; Nc1ncnc2c1c(-c1ccc(NC(=O)Nc3cc(C4(C(F)(F)F)CC4)no3)c(F)c1)cn2-c1ccncc1 ; CN1CCC(n2cc(-c3ccc(NC(=O)Nc4cc(C5(C(F)(F)F)CC5)no4)c(F)c3)c3c(N)ncnc32)CC1]

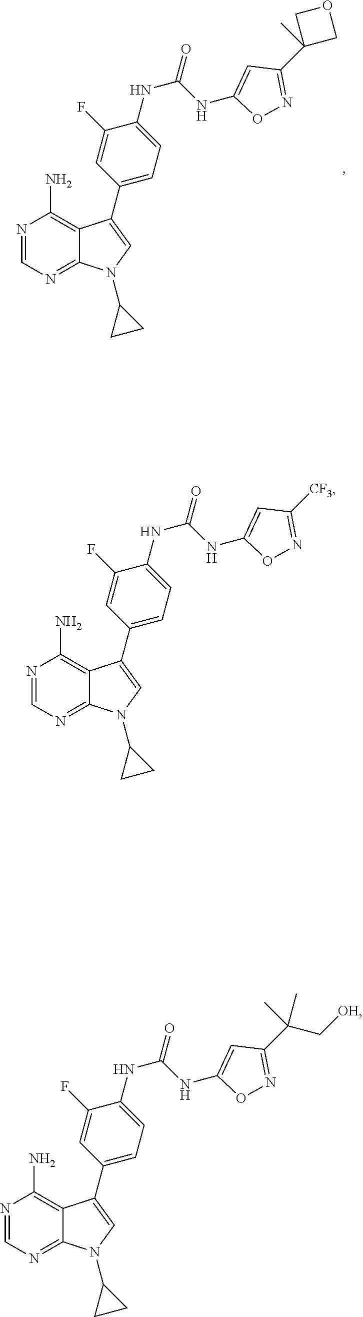 [SMILES: CC1(c2cc(NC(=O)Nc3ccc(-c4cn(C5CC5)c5ncnc(N)c45)cc3F)on2)COC1 ; Nc1ncnc2c1c(-c1ccc(NC(=O)Nc3cc(C(F)(F)F)no3)c(F)c1)cn2C1CC1 ; CC(C)(CO)c1cc(NC(=O)Nc2ccc(-c3cn(C4CC4)c4ncnc(N)c34)cc2F)on1]

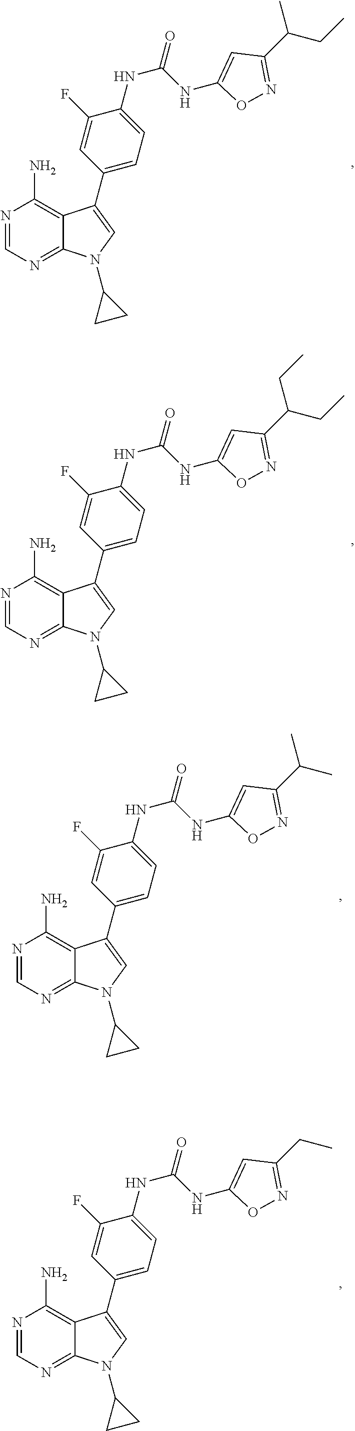 [SMILES: CCC(C)c1cc(NC(=O)Nc2ccc(-c3cn(C4CC4)c4ncnc(N)c34)cc2F)on1 ; CCC(CC)c1cc(NC(=O)Nc2ccc(-c3cn(C4CC4)c4ncnc(N)c34)cc2F)on1 ; CC(C)c1cc(NC(=O)Nc2ccc(-c3cn(C4CC4)c4ncnc(N)c34)cc2F)on1 ; CCc1cc(NC(=O)Nc2ccc(-c3cn(C4CC4)c4ncnc(N)c34)cc2F)on1]

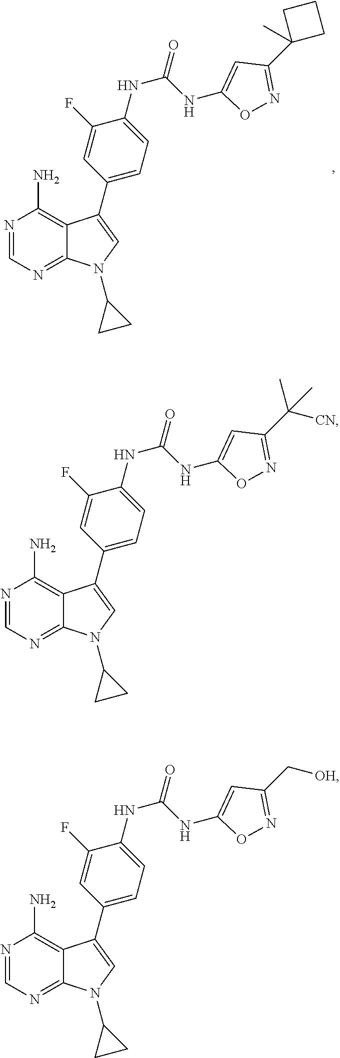 [SMILES: CC1(c2cc(NC(=O)Nc3ccc(-c4cn(C5CC5)c5ncnc(N)c45)cc3F)on2)CCC1 ; CC(C)(C#N)c1cc(NC(=O)Nc2ccc(-c3cn(C4CC4)c4ncnc(N)c34)cc2F)on1 ; Nc1ncnc2c1c(-c1ccc(NC(=O)Nc3cc(CO)no3)c(F)c1)cn2C1CC1]

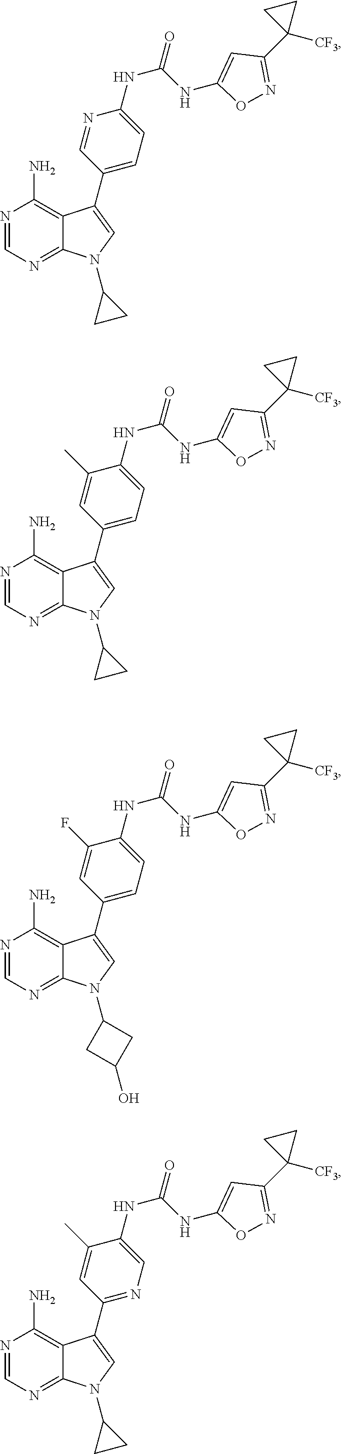 [SMILES: Nc1ncnc2c1c(-c1ccc(NC(=O)Nc3cc(C4(C(F)(F)F)CC4)no3)nc1)cn2C1CC1 ; Cc1cc(-c2cn(C3CC3)c3ncnc(N)c23)ccc1NC(=O)Nc1cc(C2(C(F)(F)F)CC2)no1 ; Nc1ncnc2c1c(-c1ccc(NC(=O)Nc3cc(C4(C(F)(F)F)CC4)no3)c(F)c1)cn2C1CC(O)C1 ; Cc1cc(-c2cn(C3CC3)c3ncnc(N)c23)ncc1NC(=O)Nc1cc(C2(C(F)(F)F)CC2)no1]

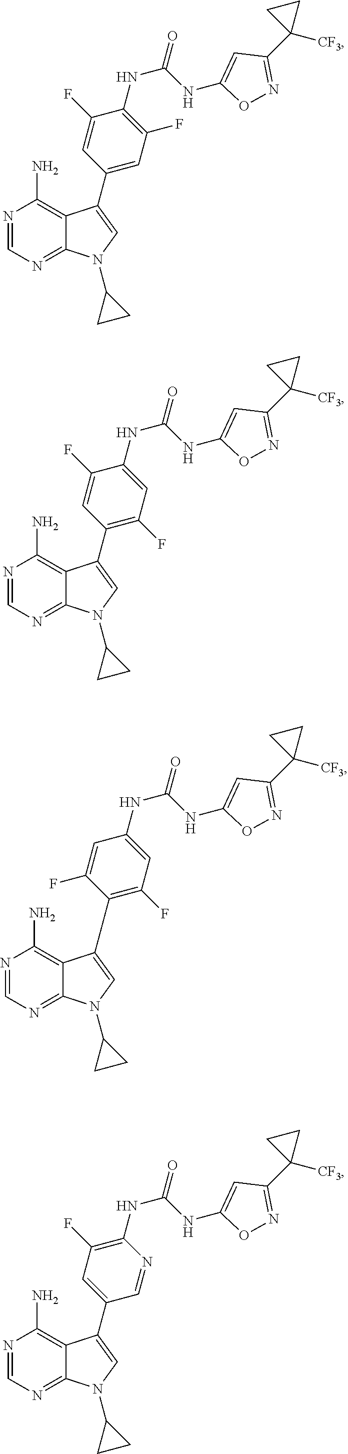 [SMILES: Nc1ncnc2c1c(-c1cc(F)c(NC(=O)Nc3cc(C4(C(F)(F)F)CC4)no3)c(F)c1)cn2C1CC1 ; Nc1ncnc2c1c(-c1cc(F)c(NC(=O)Nc3cc(C4(C(F)(F)F)CC4)no3)cc1F)cn2C1CC1 ; Nc1ncnc2c1c(-c1c(F)cc(NC(=O)Nc3cc(C4(C(F)(F)F)CC4)no3)cc1F)cn2C1CC1 ; Nc1ncnc2c1c(-c1cnc(NC(=O)Nc3cc(C4(C(F)(F)F)CC4)no3)c(F)c1)cn2C1CC1]

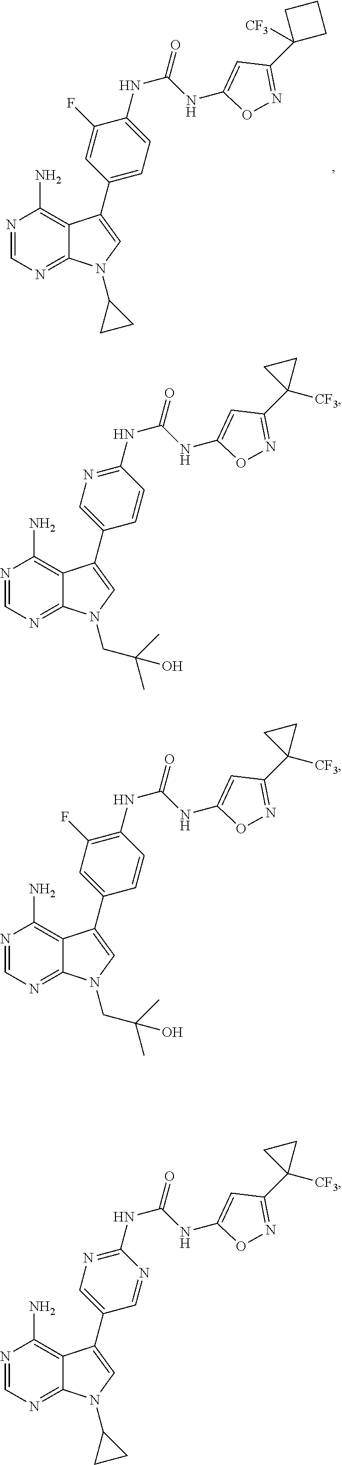 [SMILES: Nc1ncnc2c1c(-c1ccc(NC(=O)Nc3cc(C4(C(F)(F)F)CCC4)no3)c(F)c1)cn2C1CC1 ; CC(C)(O)Cn1cc(-c2ccc(NC(=O)Nc3cc(C4(C(F)(F)F)CC4)no3)nc2)c2c(N)ncnc21 ; CC(C)(O)Cn1cc(-c2ccc(NC(=O)Nc3cc(C4(C(F)(F)F)CC4)no3)c(F)c2)c2c(N)ncnc21 ; Nc1ncnc2c1c(-c1cnc(NC(=O)Nc3cc(C4(C(F)(F)F)CC4)no3)nc1)cn2C1CC1]

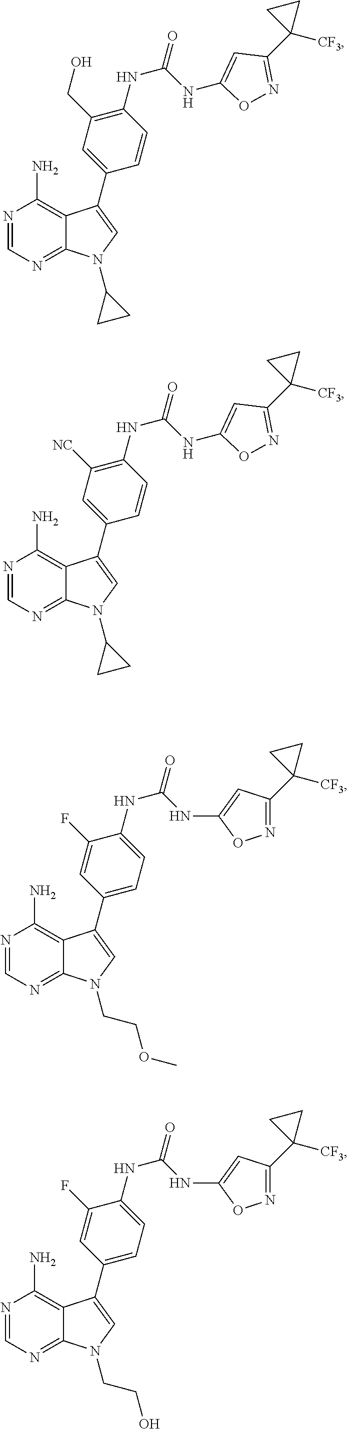 [SMILES: Nc1ncnc2c1c(-c1ccc(NC(=O)Nc3cc(C4(C(F)(F)F)CC4)no3)c(CO)c1)cn2C1CC1 ; N#Cc1cc(-c2cn(C3CC3)c3ncnc(N)c23)ccc1NC(=O)Nc1cc(C2(C(F)(F)F)CC2)no1 ; COCCn1cc(-c2ccc(NC(=O)Nc3cc(C4(C(F)(F)F)CC4)no3)c(F)c2)c2c(N)ncnc21 ; Nc1ncnc2c1c(-c1ccc(NC(=O)Nc3cc(C4(C(F)(F)F)CC4)no3)c(F)c1)cn2CCO]

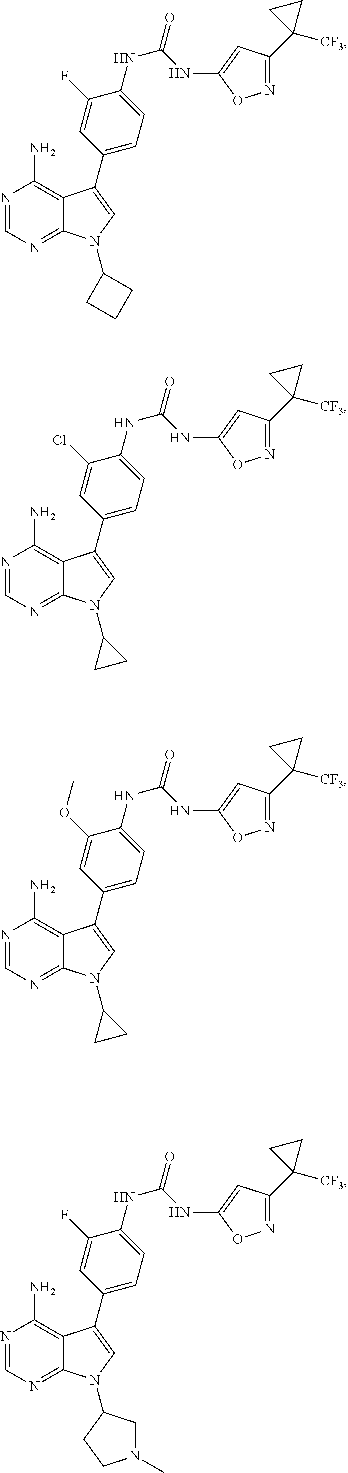 [SMILES: Nc1ncnc2c1c(-c1ccc(NC(=O)Nc3cc(C4(C(F)(F)F)CC4)no3)c(F)c1)cn2C1CCC1 ; Nc1ncnc2c1c(-c1ccc(NC(=O)Nc3cc(C4(C(F)(F)F)CC4)no3)c(Cl)c1)cn2C1CC1 ; COc1cc(-c2cn(C3CC3)c3ncnc(N)c23)ccc1NC(=O)Nc1cc(C2(C(F)(F)F)CC2)no1 ; CN1CCC(n2cc(-c3ccc(NC(=O)Nc4cc(C5(C(F)(F)F)CC5)no4)c(F)c3)c3c(N)ncnc32)C1]

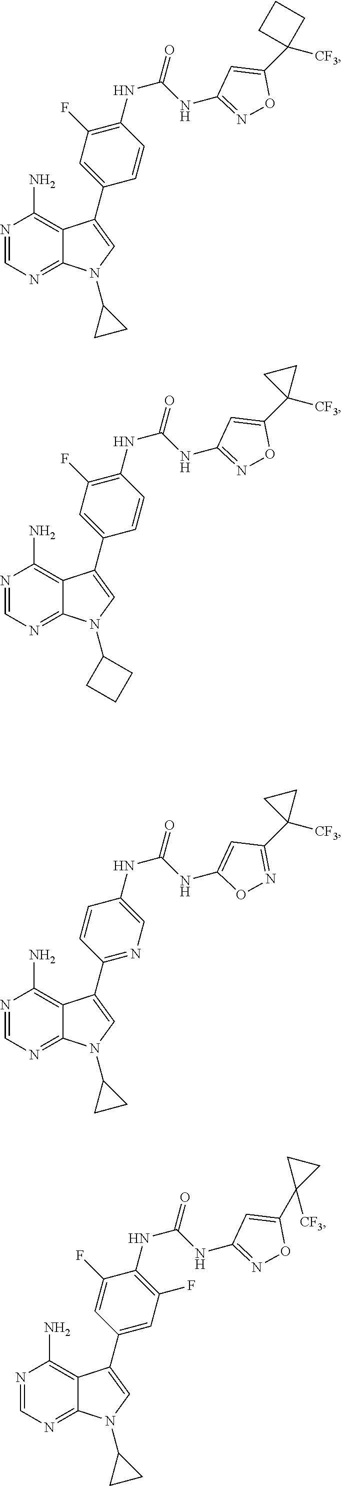 [SMILES: Nc1ncnc2c1c(-c1ccc(NC(=O)Nc3cc(C4(C(F)(F)F)CCC4)on3)c(F)c1)cn2C1CC1 ; Nc1ncnc2c1c(-c1ccc(NC(=O)Nc3cc(C4(C(F)(F)F)CC4)on3)c(F)c1)cn2C1CCC1 ; Nc1ncnc2c1c(-c1ccc(NC(=O)Nc3cc(C4(C(F)(F)F)CC4)no3)cn1)cn2C1CC1 ; Nc1ncnc2c1c(-c1cc(F)c(NC(=O)Nc3cc(C4(C(F)(F)F)CC4)on3)c(F)c1)cn2C1CC1]

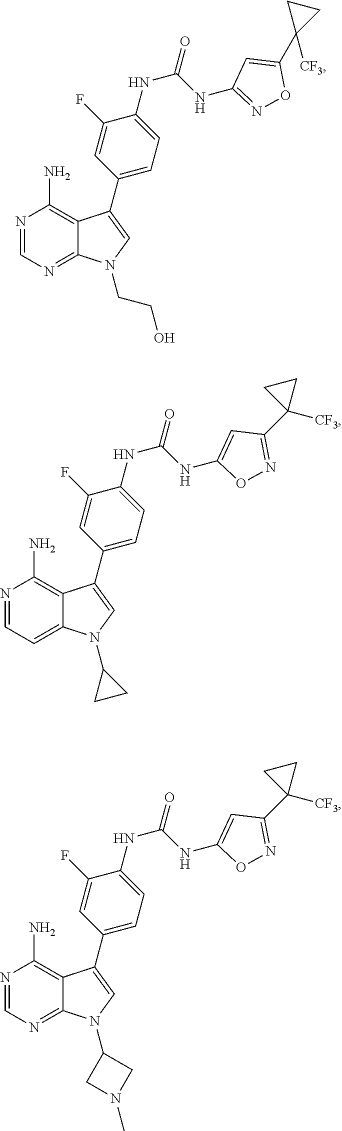 [SMILES: Nc1ncnc2c1c(-c1ccc(NC(=O)Nc3cc(C4(C(F)(F)F)CC4)on3)c(F)c1)cn2CCO ; Nc1nccc2c1c(-c1ccc(NC(=O)Nc3cc(C4(C(F)(F)F)CC4)no3)c(F)c1)cn2C1CC1 ; CN1CC(n2cc(-c3ccc(NC(=O)Nc4cc(C5(C(F)(F)F)CC5)no4)c(F)c3)c3c(N)ncnc32)C1]

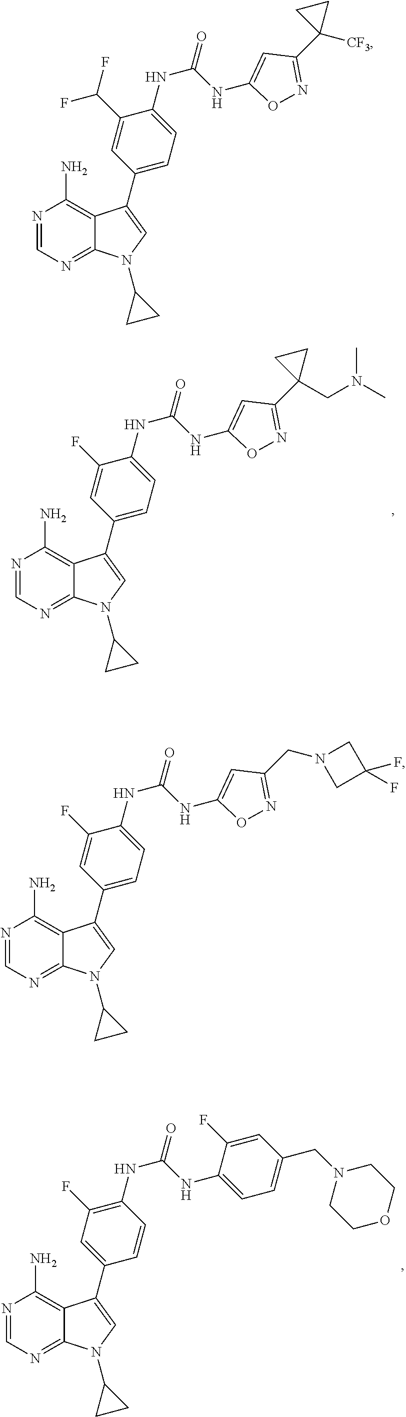 [SMILES: Nc1ncnc2c1c(-c1ccc(NC(=O)Nc3cc(C4(C(F)(F)F)CC4)no3)c(C(F)F)c1)cn2C1CC1 ; CN(C)CC1(c2cc(NC(=O)Nc3ccc(-c4cn(C5CC5)c5ncnc(N)c45)cc3F)on2)CC1 ; Nc1ncnc2c1c(-c1ccc(NC(=O)Nc3cc(CN4CC(F)(F)C4)no3)c(F)c1)cn2C1CC1 ; Nc1ncnc2c1c(-c1ccc(NC(=O)Nc3ccc(CN4CCOCC4)cc3F)c(F)c1)cn2C1CC1]

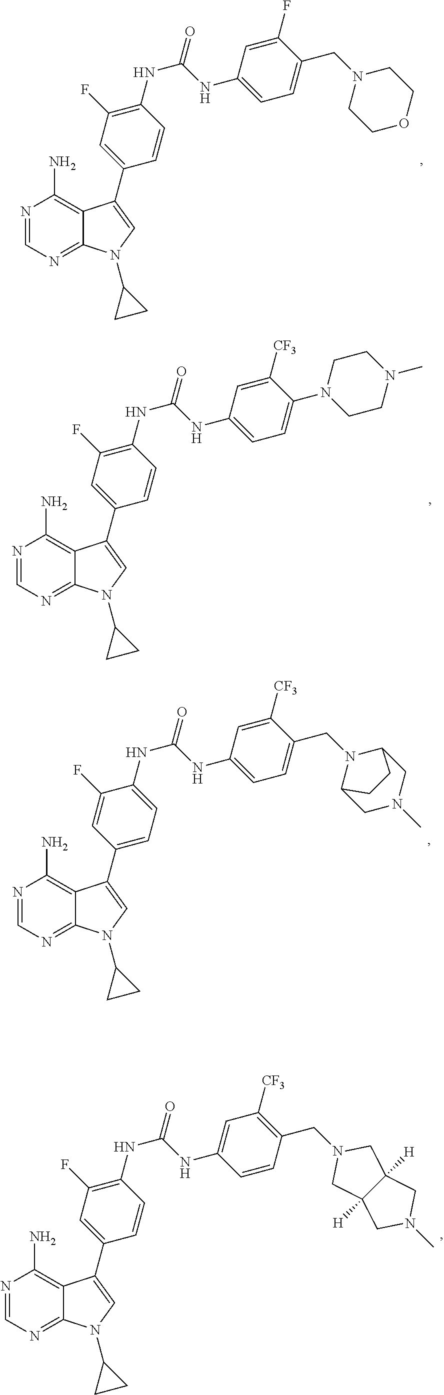 [SMILES: Nc1ncnc2c1c(-c1ccc(NC(=O)Nc3ccc(CN4CCOCC4)c(F)c3)c(F)c1)cn2C1CC1 ; CN1CCN(c2ccc(NC(=O)Nc3ccc(-c4cn(C5CC5)c5ncnc(N)c45)cc3F)cc2C(F)(F)F)CC1 ; CN1CC2CCC(C1)N2Cc1ccc(NC(=O)Nc2ccc(-c3cn(C4CC4)c4ncnc(N)c34)cc2F)cc1C(F)(F)F ; CN1C[C@@H]2CN(Cc3ccc(NC(=O)Nc4ccc(-c5cn(C6CC6)c6ncnc(N)c56)cc4F)cc3C(F)(F)F)C[C@@H]2C1]

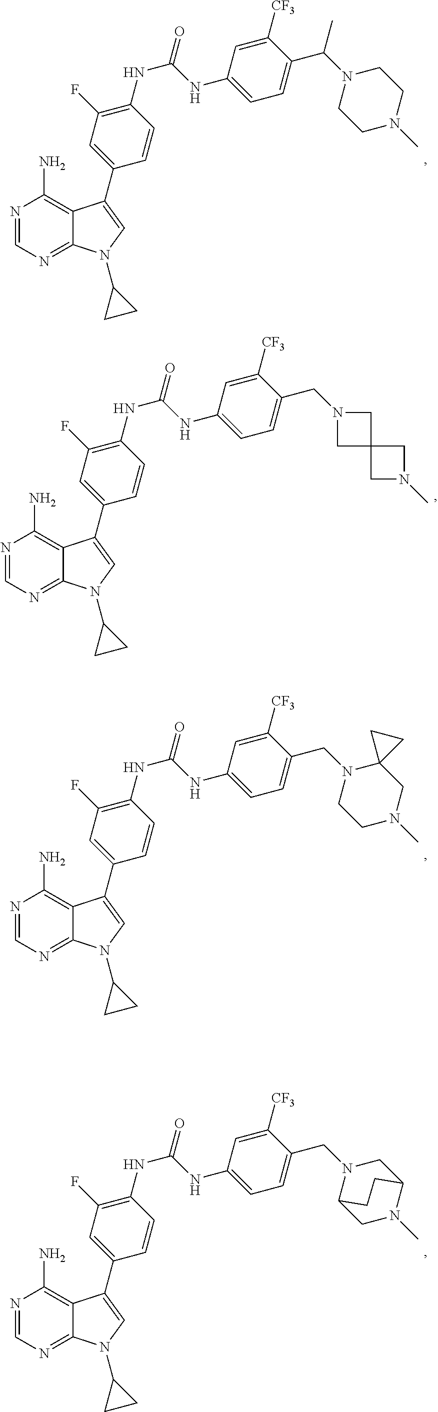 [SMILES: CC(c1ccc(NC(=O)Nc2ccc(-c3cn(C4CC4)c4ncnc(N)c34)cc2F)cc1C(F)(F)F)N1CCN(C)CC1 ; CN1CC2(C1)CN(Cc1ccc(NC(=O)Nc3ccc(-c4cn(C5CC5)c5ncnc(N)c45)cc3F)cc1C(F)(F)F)C2 ; CN1CCN(Cc2ccc(NC(=O)Nc3ccc(-c4cn(C5CC5)c5ncnc(N)c45)cc3F)cc2C(F)(F)F)C2(CC2)C1 ; CN1CC2CCC1CN2Cc1ccc(NC(=O)Nc2ccc(-c3cn(C4CC4)c4ncnc(N)c34)cc2F)cc1C(F)(F)F]

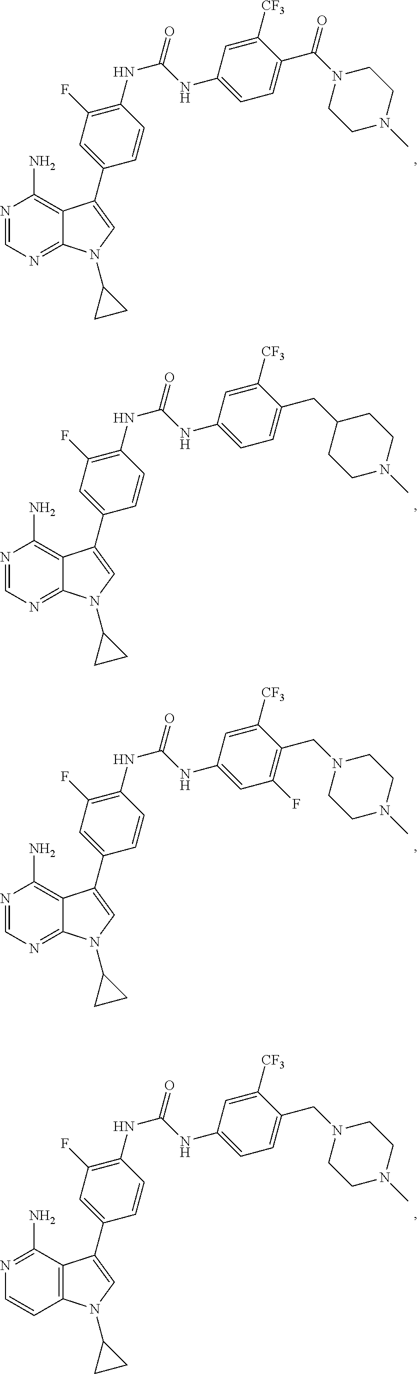 [SMILES: CN1CCN(C(=O)c2ccc(NC(=O)Nc3ccc(-c4cn(C5CC5)c5ncnc(N)c45)cc3F)cc2C(F)(F)F)CC1 ; CN1CCC(Cc2ccc(NC(=O)Nc3ccc(-c4cn(C5CC5)c5ncnc(N)c45)cc3F)cc2C(F)(F)F)CC1 ; CN1CCN(Cc2c(F)cc(NC(=O)Nc3ccc(-c4cn(C5CC5)c5ncnc(N)c45)cc3F)cc2C(F)(F)F)CC1 ; CN1CCN(Cc2ccc(NC(=O)Nc3ccc(-c4cn(C5CC5)c5ccnc(N)c45)cc3F)cc2C(F)(F)F)CC1]

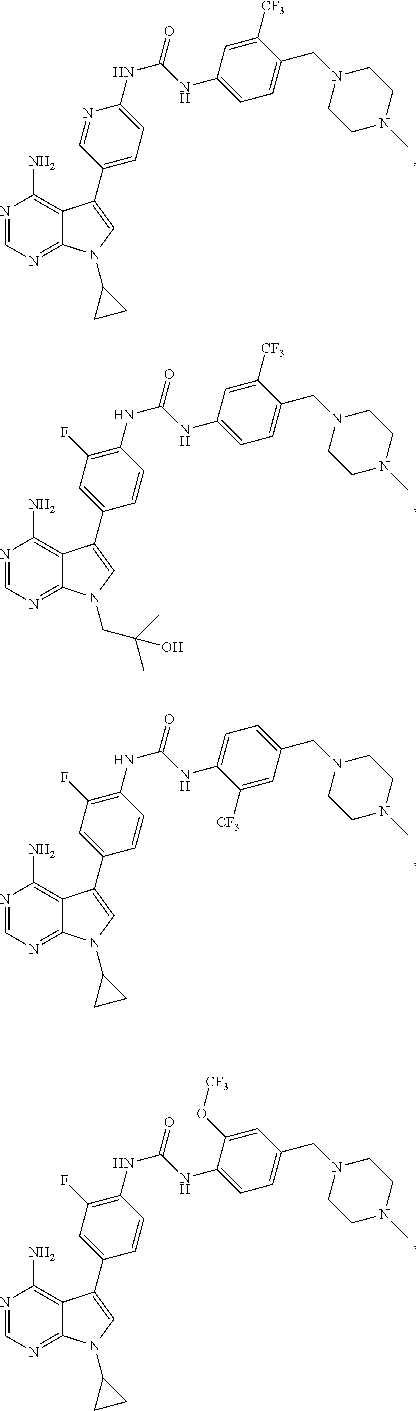 [SMILES: CN1CCN(Cc2ccc(NC(=O)Nc3ccc(-c4cn(C5CC5)c5ncnc(N)c45)cn3)cc2C(F)(F)F)CC1 ; CN1CCN(Cc2ccc(NC(=O)Nc3ccc(-c4cn(CC(C)(C)O)c5ncnc(N)c45)cc3F)cc2C(F)(F)F)CC1 ; CN1CCN(Cc2ccc(NC(=O)Nc3ccc(-c4cn(C5CC5)c5ncnc(N)c45)cc3F)c(C(F)(F)F)c2)CC1 ; CN1CCN(Cc2ccc(NC(=O)Nc3ccc(-c4cn(C5CC5)c5ncnc(N)c45)cc3F)c(OC(F)(F)F)c2)CC1]

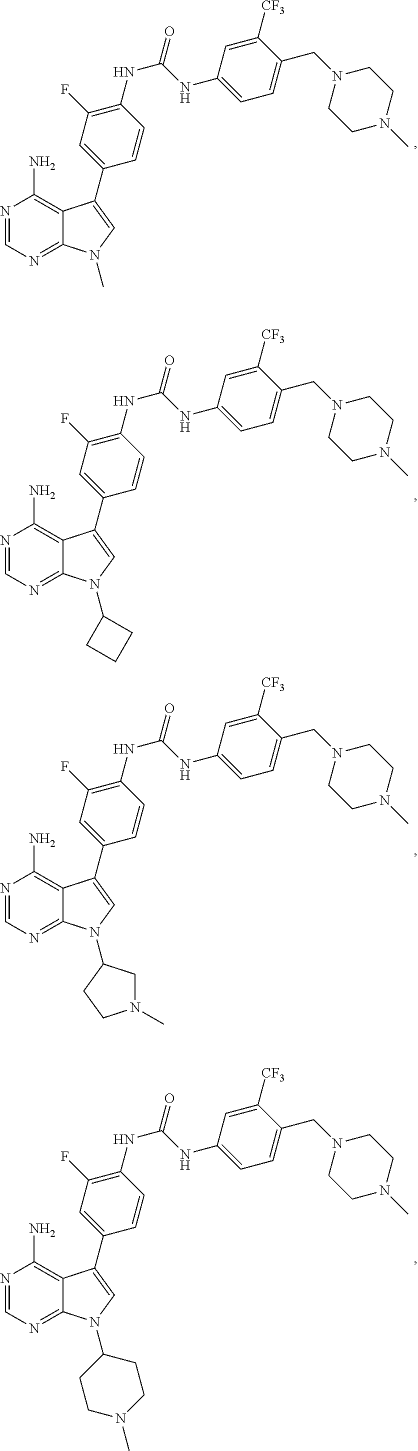 [SMILES: CN1CCN(Cc2ccc(NC(=O)Nc3ccc(-c4cn(C)c5ncnc(N)c45)cc3F)cc2C(F)(F)F)CC1 ; CN1CCN(Cc2ccc(NC(=O)Nc3ccc(-c4cn(C5CCC5)c5ncnc(N)c45)cc3F)cc2C(F)(F)F)CC1 ; CN1CCN(Cc2ccc(NC(=O)Nc3ccc(-c4cn(C5CCN(C)C5)c5ncnc(N)c45)cc3F)cc2C(F)(F)F)CC1 ; CN1CCC(n2cc(-c3ccc(NC(=O)Nc4ccc(CN5CCN(C)CC5)c(C(F)(F)F)c4)c(F)c3)c3c(N)ncnc32)CC1]

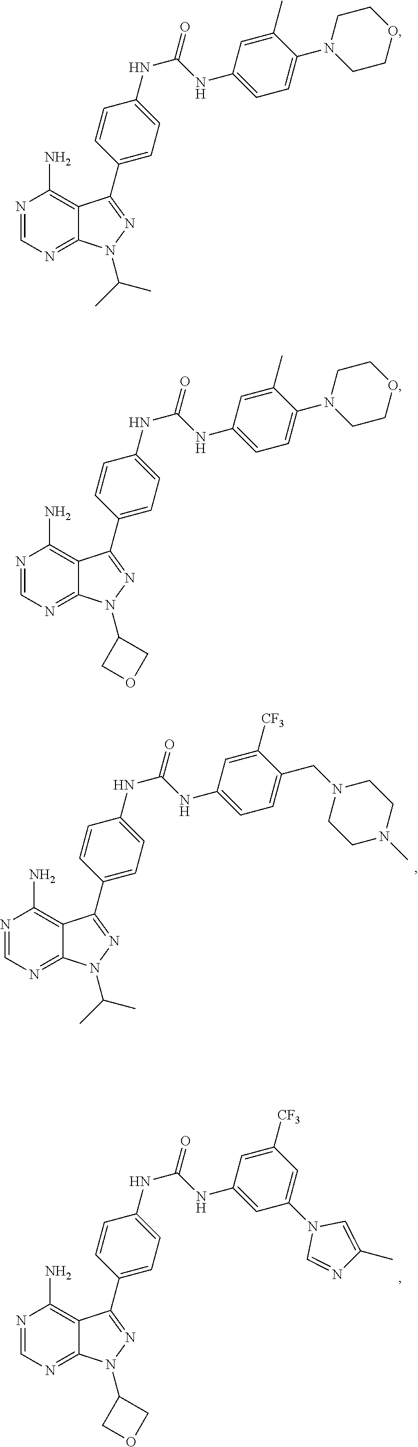 [SMILES: Cc1cc(NC(=O)Nc2ccc(-c3nn(C(C)C)c4ncnc(N)c34)cc2)ccc1N1CCOCC1 ; Cc1cc(NC(=O)Nc2ccc(-c3nn(C4COC4)c4ncnc(N)c34)cc2)ccc1N1CCOCC1 ; CC(C)n1nc(-c2ccc(NC(=O)Nc3ccc(CN4CCN(C)CC4)c(C(F)(F)F)c3)cc2)c2c(N)ncnc21 ; Cc1cn(-c2cc(NC(=O)Nc3ccc(-c4nn(C5COC5)c5ncnc(N)c45)cc3)cc(C(F)(F)F)c2)cn1]

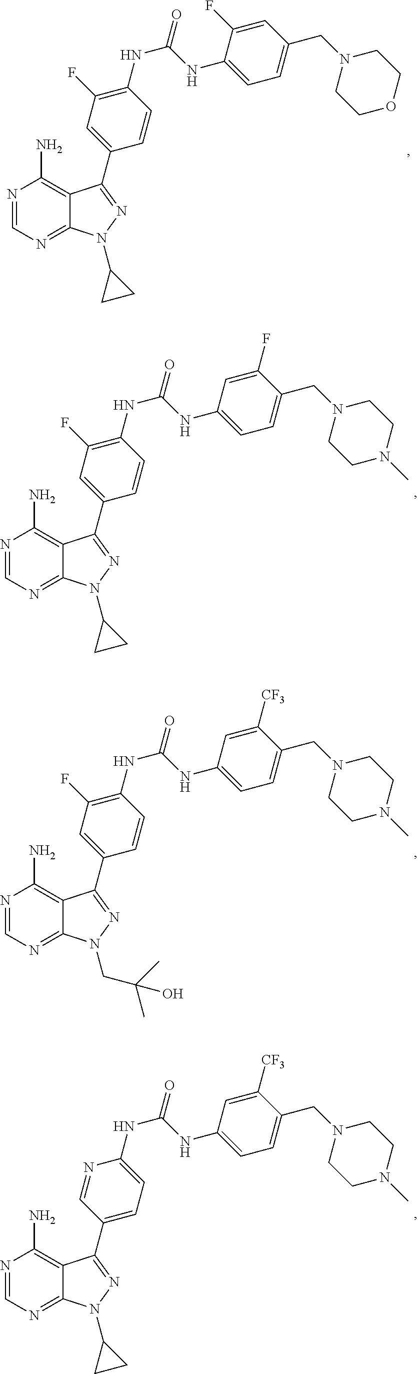 [SMILES: Nc1ncnc2c1c(-c1ccc(NC(=O)Nc3ccc(CN4CCOCC4)cc3F)c(F)c1)nn2C1CC1 ; CN1CCN(Cc2ccc(NC(=O)Nc3ccc(-c4nn(C5CC5)c5ncnc(N)c45)cc3F)cc2F)CC1 ; CN1CCN(Cc2ccc(NC(=O)Nc3ccc(-c4nn(CC(C)(C)O)c5ncnc(N)c45)cc3F)cc2C(F)(F)F)CC1 ; CN1CCN(Cc2ccc(NC(=O)Nc3ccc(-c4nn(C5CC5)c5ncnc(N)c45)cn3)cc2C(F)(F)F)CC1]

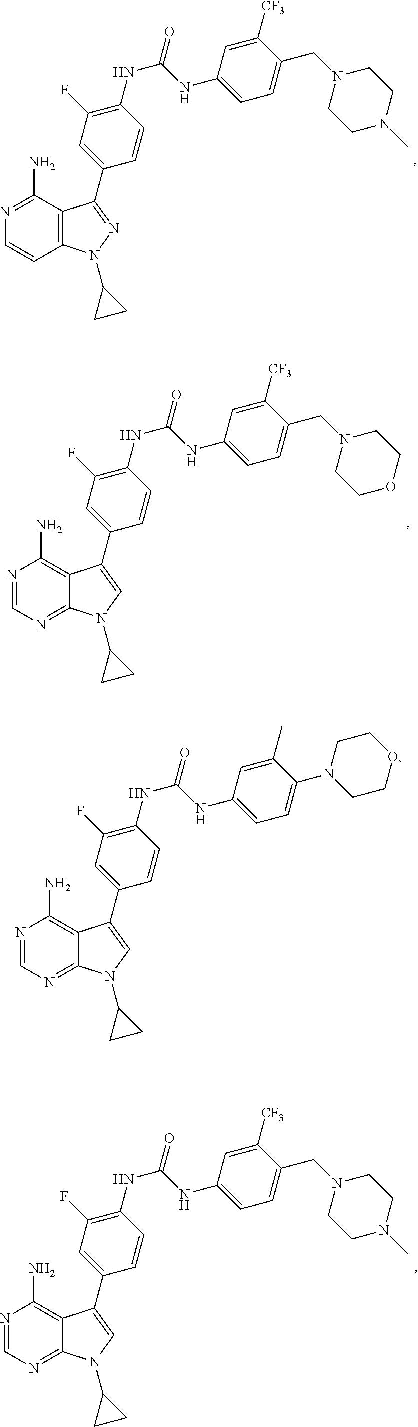 [SMILES: CN1CCN(Cc2ccc(NC(=O)Nc3ccc(-c4nn(C5CC5)c5ccnc(N)c45)cc3F)cc2C(F)(F)F)CC1 ; Nc1ncnc2c1c(-c1ccc(NC(=O)Nc3ccc(CN4CCOCC4)c(C(F)(F)F)c3)c(F)c1)cn2C1CC1 ; Cc1cc(NC(=O)Nc2ccc(-c3cn(C4CC4)c4ncnc(N)c34)cc2F)ccc1N1CCOCC1 ; CN1CCN(Cc2ccc(NC(=O)Nc3ccc(-c4cn(C5CC5)c5ncnc(N)c45)cc3F)cc2C(F)(F)F)CC1]

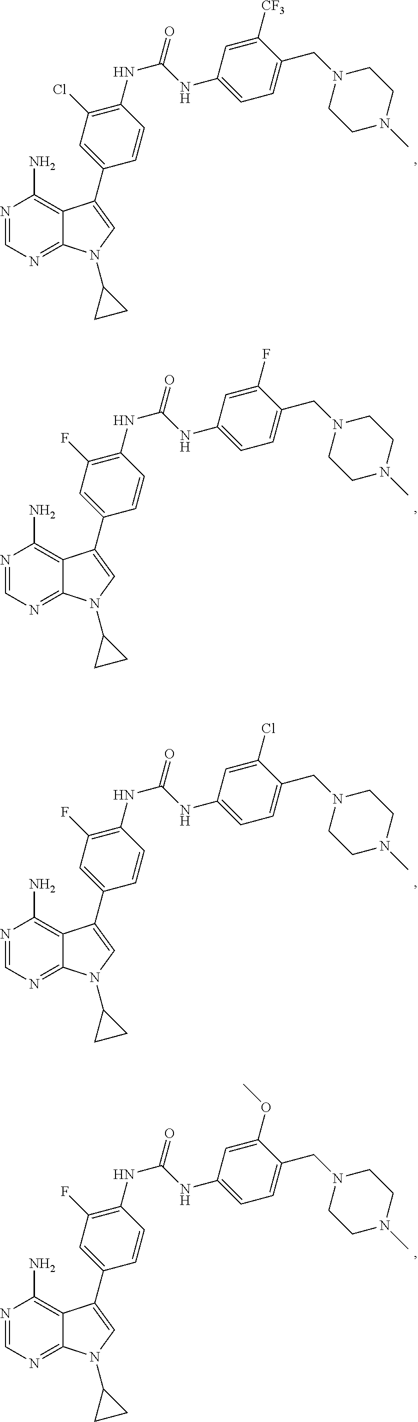 [SMILES: CN1CCN(Cc2ccc(NC(=O)Nc3ccc(-c4cn(C5CC5)c5ncnc(N)c45)cc3Cl)cc2C(F)(F)F)CC1 ; CN1CCN(Cc2ccc(NC(=O)Nc3ccc(-c4cn(C5CC5)c5ncnc(N)c45)cc3F)cc2F)CC1 ; CN1CCN(Cc2ccc(NC(=O)Nc3ccc(-c4cn(C5CC5)c5ncnc(N)c45)cc3F)cc2Cl)CC1 ; COc1cc(NC(=O)Nc2ccc(-c3cn(C4CC4)c4ncnc(N)c34)cc2F)ccc1CN1CCN(C)CC1]

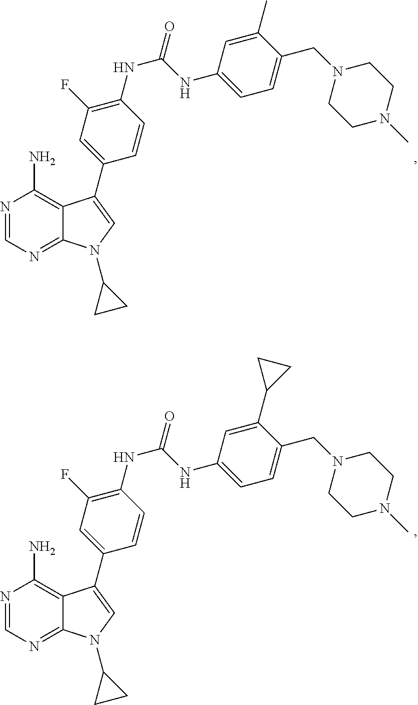 [SMILES: Cc1cc(NC(=O)Nc2ccc(-c3cn(C4CC4)c4ncnc(N)c34)cc2F)ccc1CN1CCN(C)CC1 ; CN1CCN(Cc2ccc(NC(=O)Nc3ccc(-c4cn(C5CC5)c5ncnc(N)c45)cc3F)cc2C2CC2)CC1]

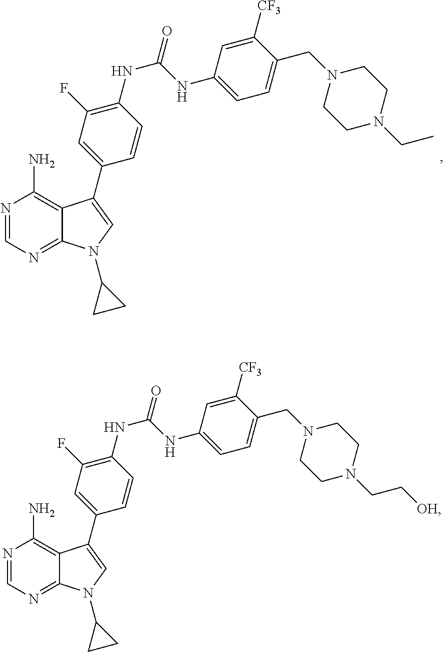 [SMILES: CCN1CCN(Cc2ccc(NC(=O)Nc3ccc(-c4cn(C5CC5)c5ncnc(N)c45)cc3F)cc2C(F)(F)F)CC1 ; Nc1ncnc2c1c(-c1ccc(NC(=O)Nc3ccc(CN4CCN(CCO)CC4)c(C(F)(F)F)c3)c(F)c1)cn2C1CC1]

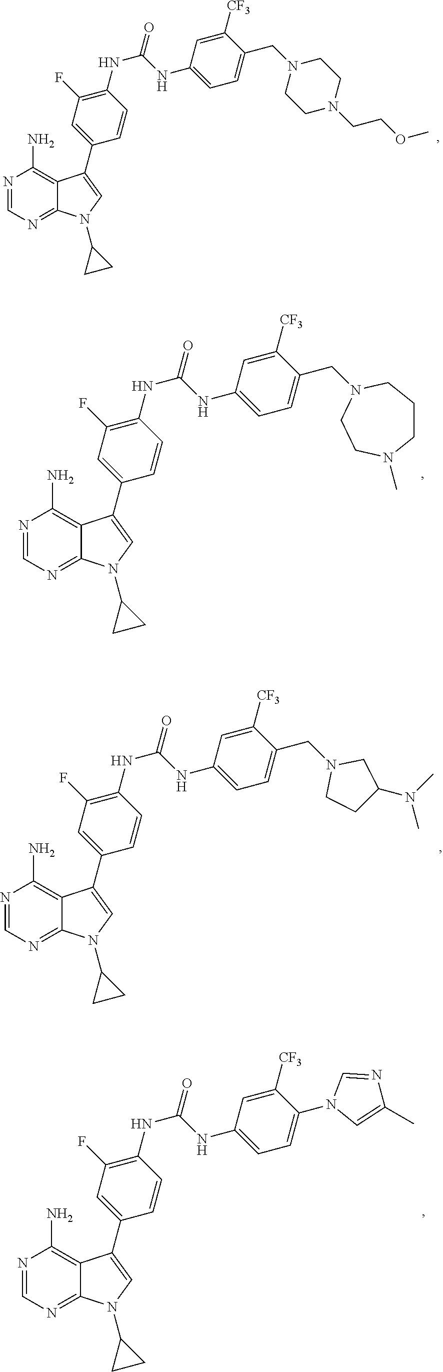 [SMILES: COCCN1CCN(Cc2ccc(NC(=O)Nc3ccc(-c4cn(C5CC5)c5ncnc(N)c45)cc3F)cc2C(F)(F)F)CC1 ; CN1CCCN(Cc2ccc(NC(=O)Nc3ccc(-c4cn(C5CC5)c5ncnc(N)c45)cc3F)cc2C(F)(F)F)CC1 ; CN(C)C1CCN(Cc2ccc(NC(=O)Nc3ccc(-c4cn(C5CC5)c5ncnc(N)c45)cc3F)cc2C(F)(F)F)C1 ; Cc1cn(-c2ccc(NC(=O)Nc3ccc(-c4cn(C5CC5)c5ncnc(N)c45)cc3F)cc2C(F)(F)F)cn1]

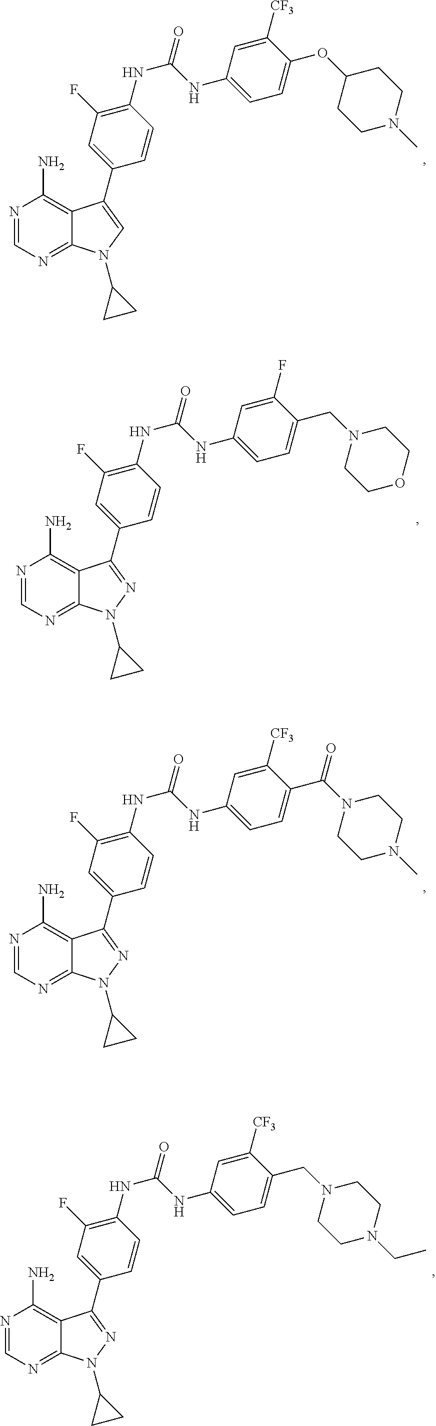 [SMILES: CN1CCC(Oc2ccc(NC(=O)Nc3ccc(-c4cn(C5CC5)c5ncnc(N)c45)cc3F)cc2C(F)(F)F)CC1 ; Nc1ncnc2c1c(-c1ccc(NC(=O)Nc3ccc(CN4CCOCC4)c(F)c3)c(F)c1)nn2C1CC1 ; CN1CCN(C(=O)c2ccc(NC(=O)Nc3ccc(-c4nn(C5CC5)c5ncnc(N)c45)cc3F)cc2C(F)(F)F)CC1 ; CCN1CCN(Cc2ccc(NC(=O)Nc3ccc(-c4nn(C5CC5)c5ncnc(N)c45)cc3F)cc2C(F)(F)F)CC1]

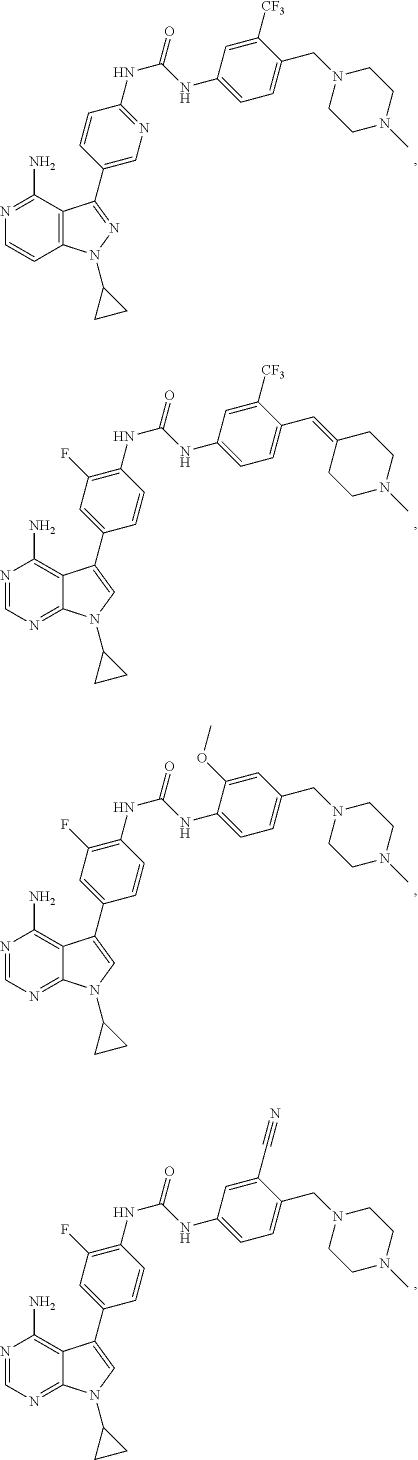 [SMILES: CN1CCN(Cc2ccc(NC(=O)Nc3ccc(-c4nn(C5CC5)c5ccnc(N)c45)cn3)cc2C(F)(F)F)CC1 ; CN1CCC(=Cc2ccc(NC(=O)Nc3ccc(-c4cn(C5CC5)c5ncnc(N)c45)cc3F)cc2C(F)(F)F)CC1 ; COc1cc(CN2CCN(C)CC2)ccc1NC(=O)Nc1ccc(-c2cn(C3CC3)c3ncnc(N)c23)cc1F ; CN1CCN(Cc2ccc(NC(=O)Nc3ccc(-c4cn(C5CC5)c5ncnc(N)c45)cc3F)cc2C#N)CC1]

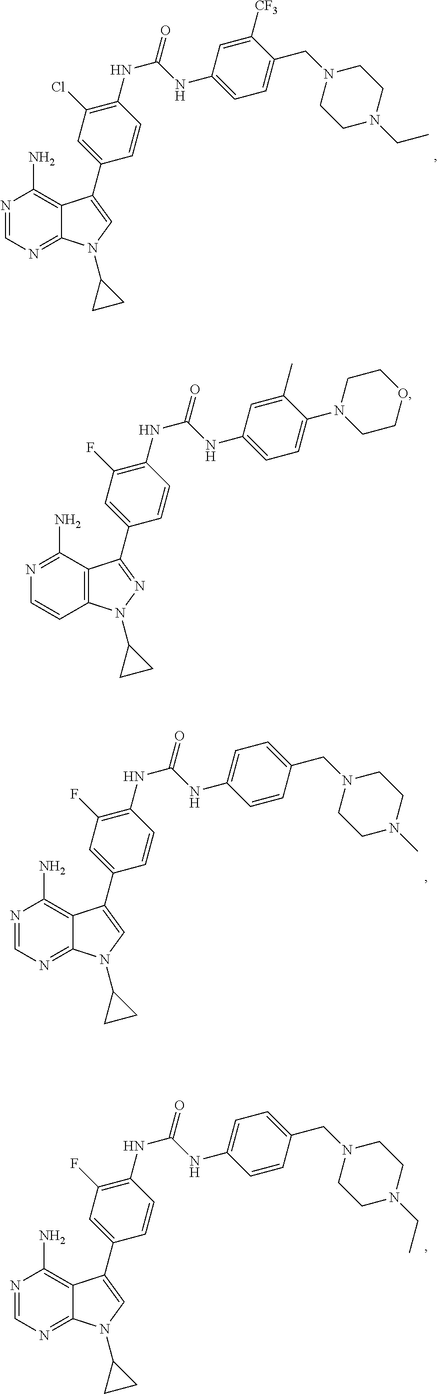 [SMILES: CCN1CCN(Cc2ccc(NC(=O)Nc3ccc(-c4cn(C5CC5)c5ncnc(N)c45)cc3Cl)cc2C(F)(F)F)CC1 ; Cc1cc(NC(=O)Nc2ccc(-c3nn(C4CC4)c4ccnc(N)c34)cc2F)ccc1N1CCOCC1 ; CN1CCN(Cc2ccc(NC(=O)Nc3ccc(-c4cn(C5CC5)c5ncnc(N)c45)cc3F)cc2)CC1 ; CCN1CCN(Cc2ccc(NC(=O)Nc3ccc(-c4cn(C5CC5)c5ncnc(N)c45)cc3F)cc2)CC1]

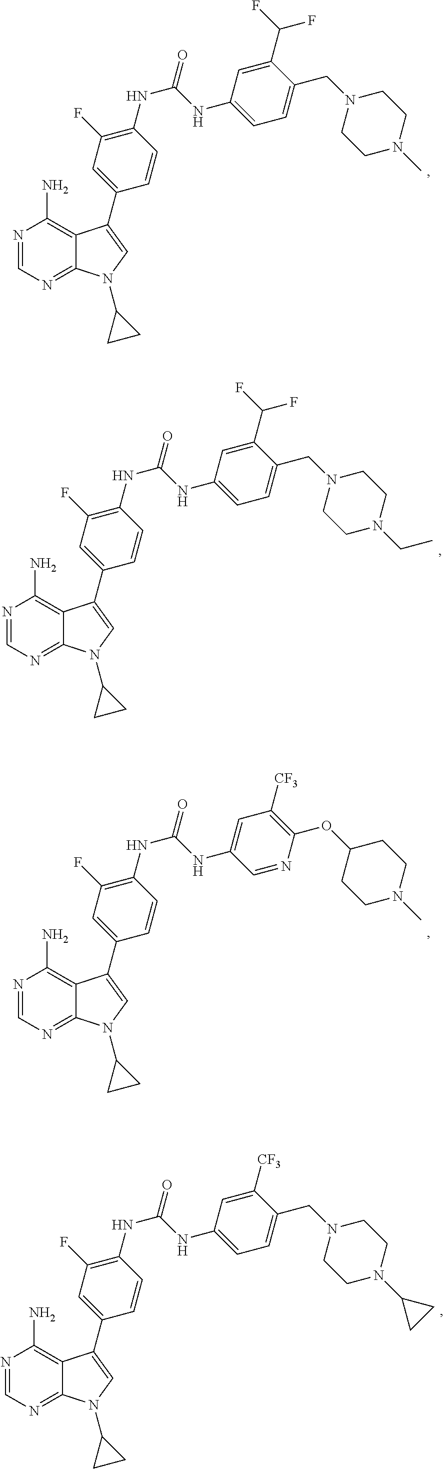 [SMILES: CN1CCN(Cc2ccc(NC(=O)Nc3ccc(-c4cn(C5CC5)c5ncnc(N)c45)cc3F)cc2C(F)F)CC1 ; CCN1CCN(Cc2ccc(NC(=O)Nc3ccc(-c4cn(C5CC5)c5ncnc(N)c45)cc3F)cc2C(F)F)CC1 ; CN1CCC(Oc2ncc(NC(=O)Nc3ccc(-c4cn(C5CC5)c5ncnc(N)c45)cc3F)cc2C(F)(F)F)CC1 ; Nc1ncnc2c1c(-c1ccc(NC(=O)Nc3ccc(CN4CCN(C5CC5)CC4)c(C(F)(F)F)c3)c(F)c1)cn2C1CC1]

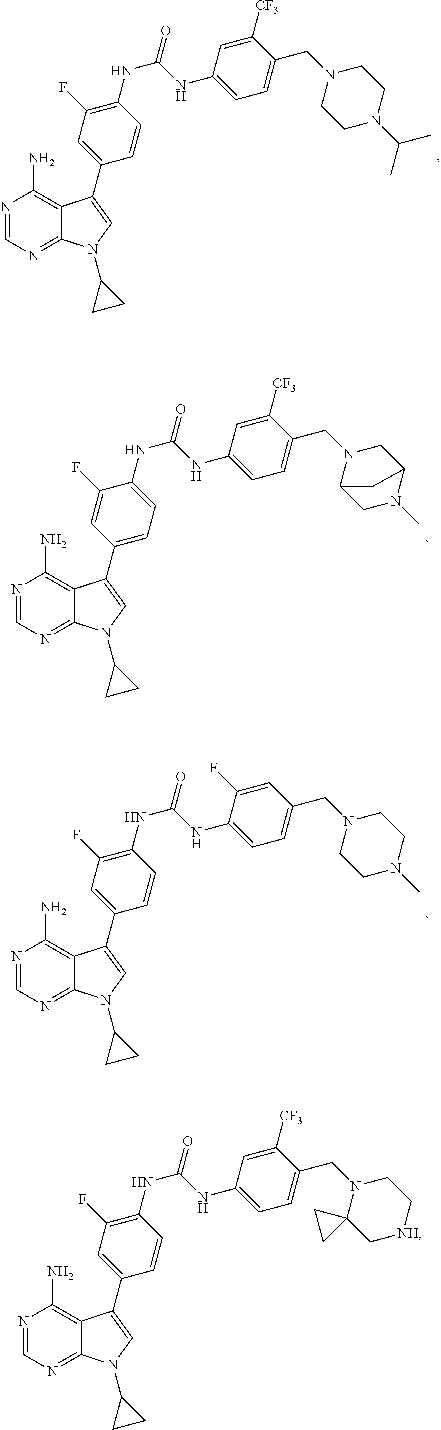 [SMILES: CC(C)N1CCN(Cc2ccc(NC(=O)Nc3ccc(-c4cn(C5CC5)c5ncnc(N)c45)cc3F)cc2C(F)(F)F)CC1 ; CN1CC2CC1CN2Cc1ccc(NC(=O)Nc2ccc(-c3cn(C4CC4)c4ncnc(N)c34)cc2F)cc1C(F)(F)F ; CN1CCN(Cc2ccc(NC(=O)Nc3ccc(-c4cn(C5CC5)c5ncnc(N)c45)cc3F)c(F)c2)CC1 ; Nc1ncnc2c1c(-c1ccc(NC(=O)Nc3ccc(CN4CCNCC45CC5)c(C(F)(F)F)c3)c(F)c1)cn2C1CC1]

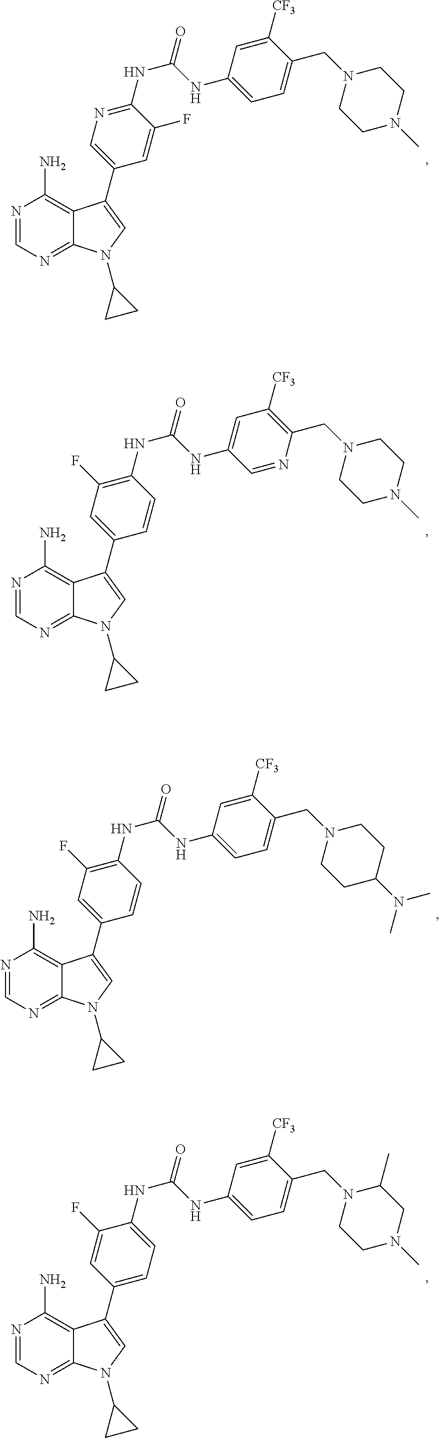 [SMILES: CN1CCN(Cc2ccc(NC(=O)Nc3ncc(-c4cn(C5CC5)c5ncnc(N)c45)cc3F)cc2C(F)(F)F)CC1 ; CN1CCN(Cc2ncc(NC(=O)Nc3ccc(-c4cn(C5CC5)c5ncnc(N)c45)cc3F)cc2C(F)(F)F)CC1 ; CN(C)C1CCN(Cc2ccc(NC(=O)Nc3ccc(-c4cn(C5CC5)c5ncnc(N)c45)cc3F)cc2C(F)(F)F)CC1 ; CC1CN(C)CCN1Cc1ccc(NC(=O)Nc2ccc(-c3cn(C4CC4)c4ncnc(N)c34)cc2F)cc1C(F)(F)F]

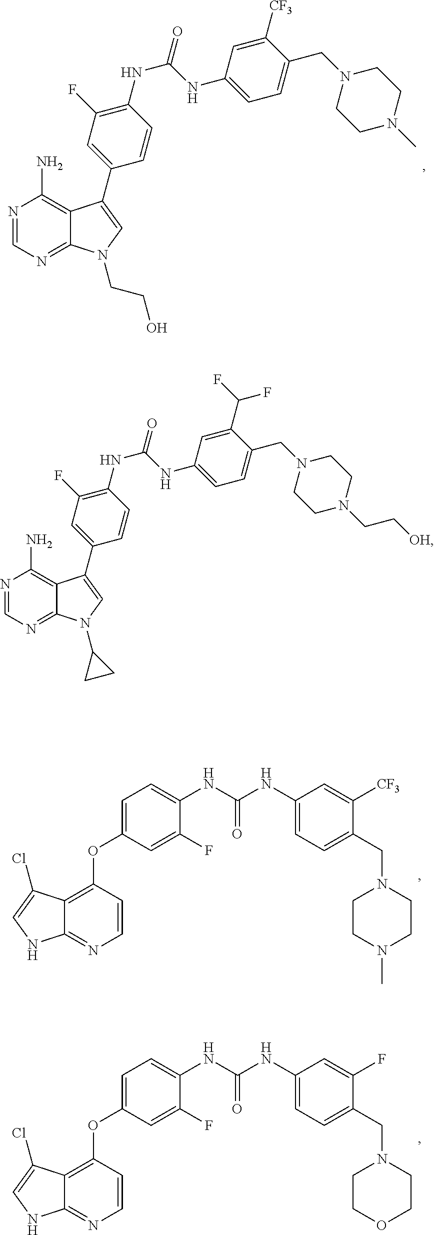 [SMILES: CN1CCN(Cc2ccc(NC(=O)Nc3ccc(-c4cn(CCO)c5ncnc(N)c45)cc3F)cc2C(F)(F)F)CC1 ; Nc1ncnc2c1c(-c1ccc(NC(=O)Nc3ccc(CN4CCN(CCO)CC4)c(C(F)F)c3)c(F)c1)cn2C1CC1 ; CN1CCN(Cc2ccc(NC(=O)Nc3ccc(Oc4ccnc5[nH]cc(Cl)c45)cc3F)cc2C(F)(F)F)CC1 ; O=C(Nc1ccc(CN2CCOCC2)c(F)c1)Nc1ccc(Oc2ccnc3[nH]cc(Cl)c23)cc1F]

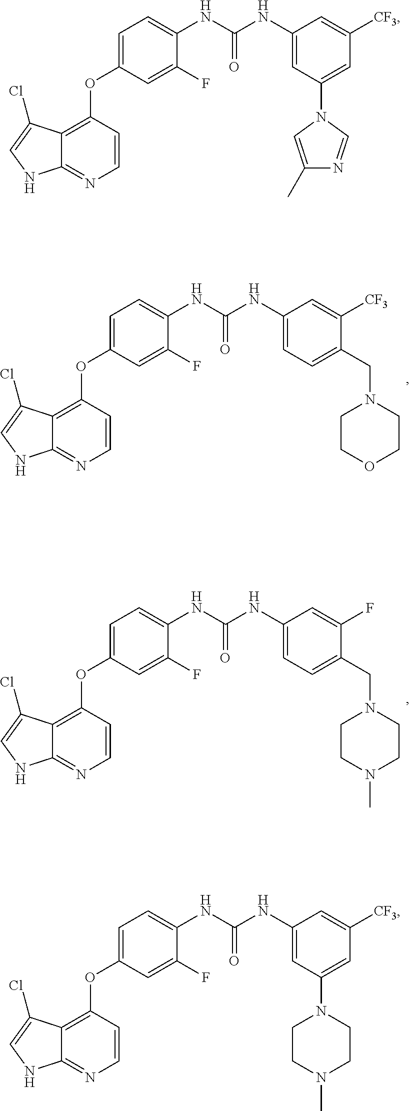 [SMILES: Cc1cn(-c2cc(NC(=O)Nc3ccc(Oc4ccnc5[nH]cc(Cl)c45)cc3F)cc(C(F)(F)F)c2)cn1 ; O=C(Nc1ccc(CN2CCOCC2)c(C(F)(F)F)c1)Nc1ccc(Oc2ccnc3[nH]cc(Cl)c23)cc1F ; CN1CCN(Cc2ccc(NC(=O)Nc3ccc(Oc4ccnc5[nH]cc(Cl)c45)cc3F)cc2F)CC1 ; CN1CCN(c2cc(NC(=O)Nc3ccc(Oc4ccnc5[nH]cc(Cl)c45)cc3F)cc(C(F)(F)F)c2)CC1]

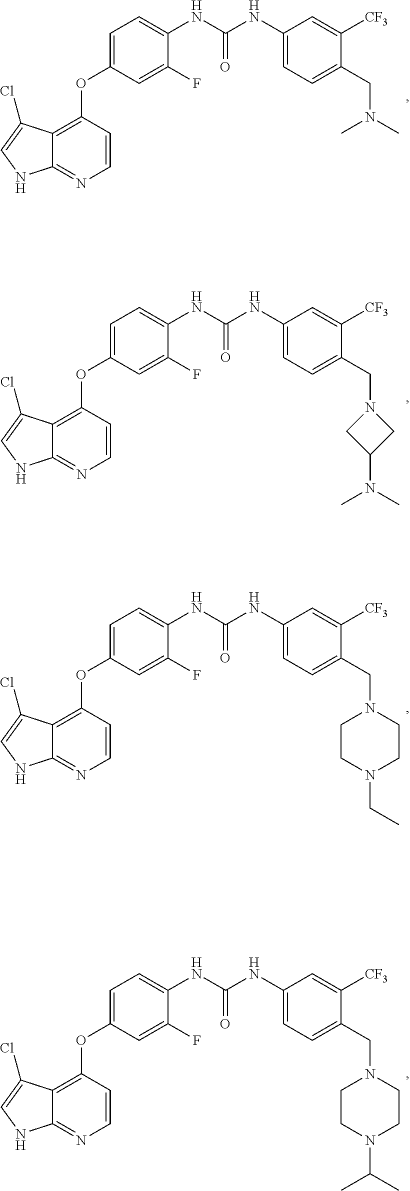 [SMILES: CN(C)Cc1ccc(NC(=O)Nc2ccc(Oc3ccnc4[nH]cc(Cl)c34)cc2F)cc1C(F)(F)F ; CN(C)C1CN(Cc2ccc(NC(=O)Nc3ccc(Oc4ccnc5[nH]cc(Cl)c45)cc3F)cc2C(F)(F)F)C1 ; CCN1CCN(Cc2ccc(NC(=O)Nc3ccc(Oc4ccnc5[nH]cc(Cl)c45)cc3F)cc2C(F)(F)F)CC1 ; CC(C)N1CCN(Cc2ccc(NC(=O)Nc3ccc(Oc4ccnc5[nH]cc(Cl)c45)cc3F)cc2C(F)(F)F)CC1]

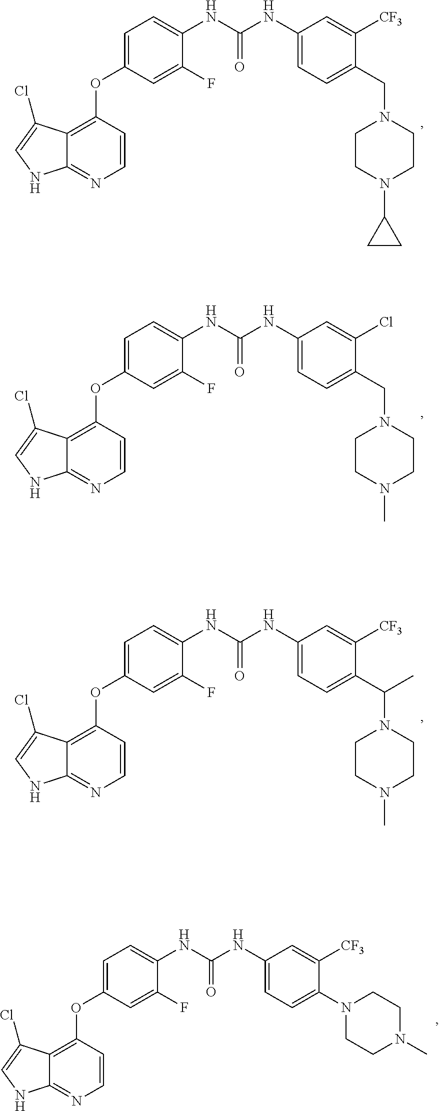 [SMILES: O=C(Nc1ccc(CN2CCN(C3CC3)CC2)c(C(F)(F)F)c1)Nc1ccc(Oc2ccnc3[nH]cc(Cl)c23)cc1F ; CN1CCN(Cc2ccc(NC(=O)Nc3ccc(Oc4ccnc5[nH]cc(Cl)c45)cc3F)cc2Cl)CC1 ; CC(c1ccc(NC(=O)Nc2ccc(Oc3ccnc4[nH]cc(Cl)c34)cc2F)cc1C(F)(F)F)N1CCN(C)CC1 ; CN1CCN(c2ccc(NC(=O)Nc3ccc(Oc4ccnc5[nH]cc(Cl)c45)cc3F)cc2C(F)(F)F)CC1]

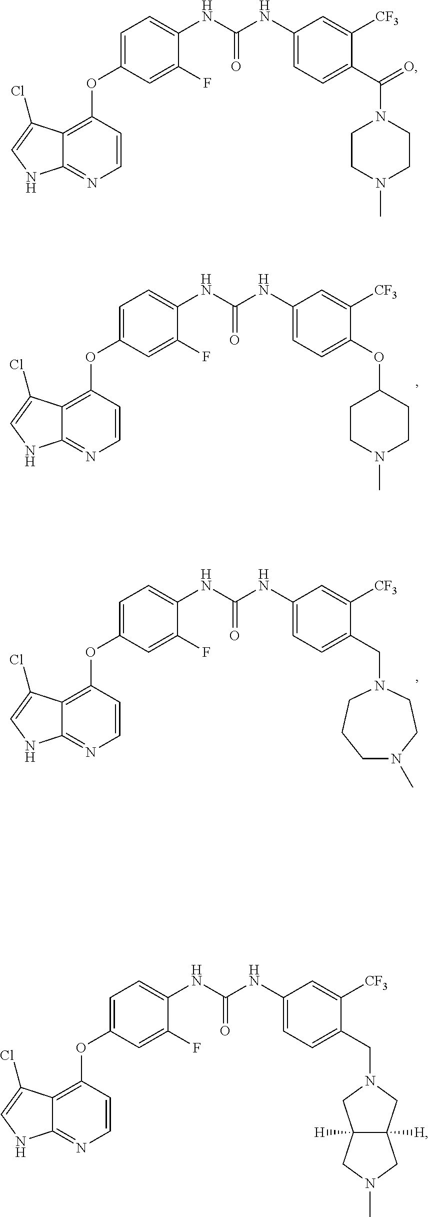

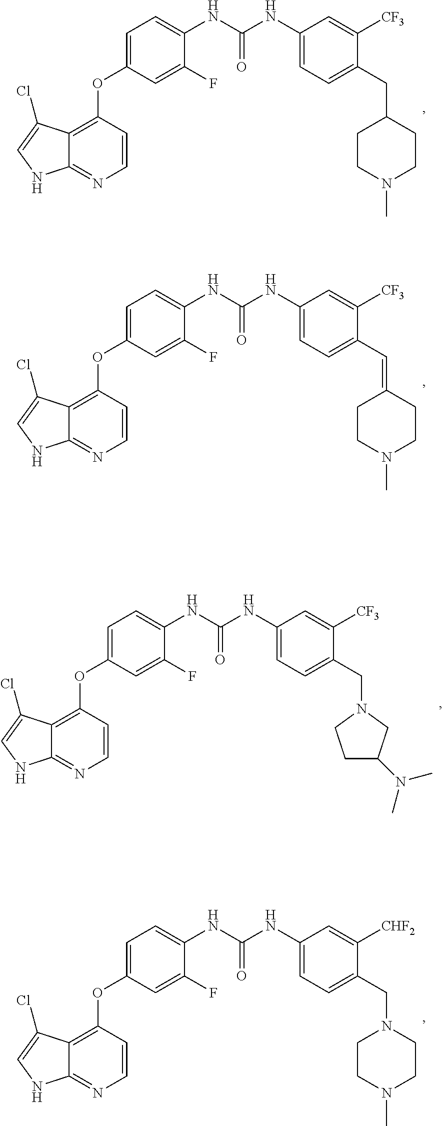 [SMILES: CN1CCC(Cc2ccc(NC(=O)Nc3ccc(Oc4ccnc5[nH]cc(Cl)c45)cc3F)cc2C(F)(F)F)CC1 ; CN1CCC(=Cc2ccc(NC(=O)Nc3ccc(Oc4ccnc5[nH]cc(Cl)c45)cc3F)cc2C(F)(F)F)CC1 ; CN(C)C1CCN(Cc2ccc(NC(=O)Nc3ccc(Oc4ccnc5[nH]cc(Cl)c45)cc3F)cc2C(F)(F)F)C1 ; CN1CCN(Cc2ccc(NC(=O)Nc3ccc(Oc4ccnc5[nH]cc(Cl)c45)cc3F)cc2C(F)F)CC1]

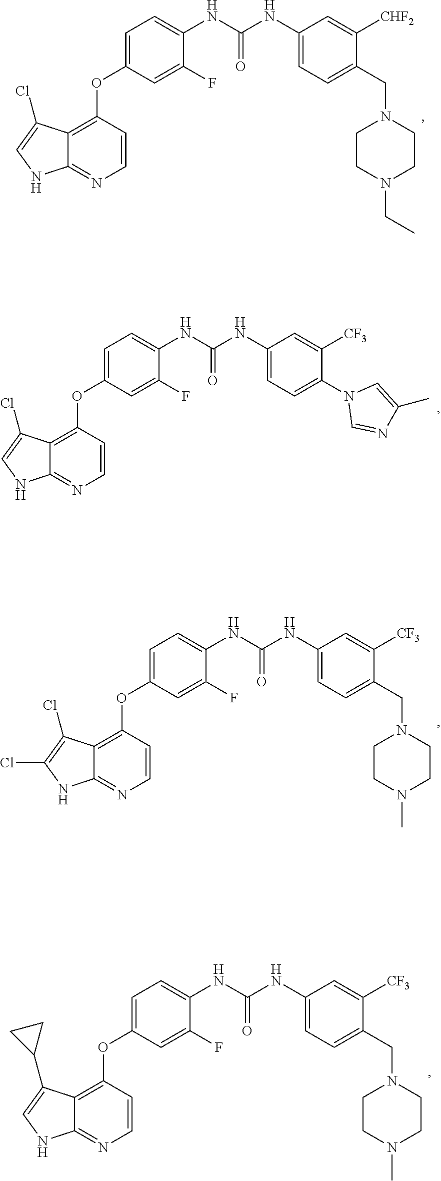 [SMILES: CCN1CCN(Cc2ccc(NC(=O)Nc3ccc(Oc4ccnc5[nH]cc(Cl)c45)cc3F)cc2C(F)F)CC1 ; Cc1cn(-c2ccc(NC(=O)Nc3ccc(Oc4ccnc5[nH]cc(Cl)c45)cc3F)cc2C(F)(F)F)cn1 ; CN1CCN(Cc2ccc(NC(=O)Nc3ccc(Oc4ccnc5[nH]c(Cl)c(Cl)c45)cc3F)cc2C(F)(F)F)CC1 ; CN1CCN(Cc2ccc(NC(=O)Nc3ccc(Oc4ccnc5[nH]cc(C6CC6)c45)cc3F)cc2C(F)(F)F)CC1]

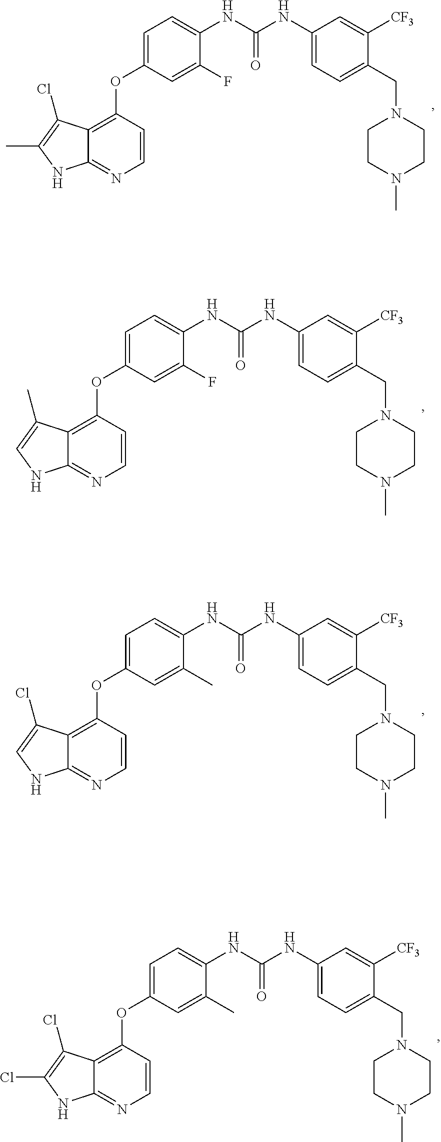 [SMILES: Cc1[nH]c2nccc(Oc3ccc(NC(=O)Nc4ccc(CN5CCN(C)CC5)c(C(F)(F)F)c4)c(F)c3)c2c1Cl ; Cc1c[nH]c2nccc(Oc3ccc(NC(=O)Nc4ccc(CN5CCN(C)CC5)c(C(F)(F)F)c4)c(F)c3)c12 ; Cc1cc(Oc2ccnc3[nH]cc(Cl)c23)ccc1NC(=O)Nc1ccc(CN2CCN(C)CC2)c(C(F)(F)F)c1 ; Cc1cc(Oc2ccnc3[nH]c(Cl)c(Cl)c23)ccc1NC(=O)Nc1ccc(CN2CCN(C)CC2)c(C(F)(F)F)c1]

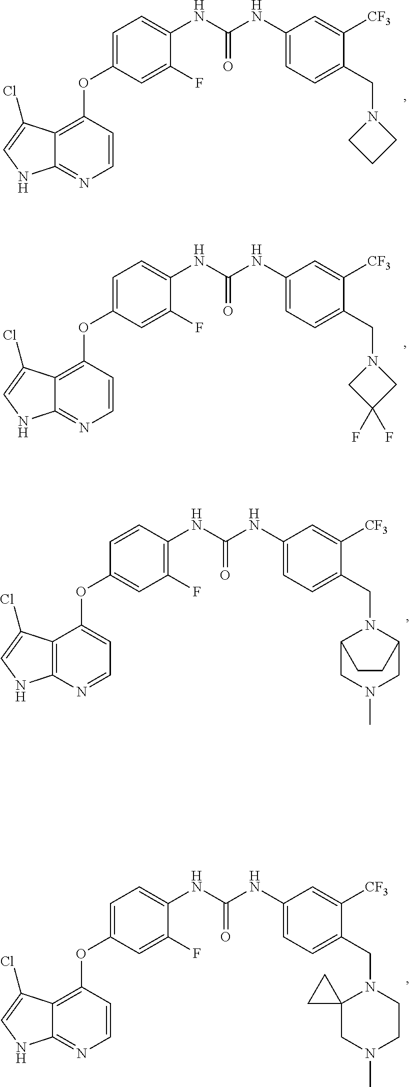 [SMILES: O=C(Nc1ccc(CN2CCC2)c(C(F)(F)F)c1)Nc1ccc(Oc2ccnc3[nH]cc(Cl)c23)cc1F ; O=C(Nc1ccc(CN2CC(F)(F)C2)c(C(F)(F)F)c1)Nc1ccc(Oc2ccnc3[nH]cc(Cl)c23)cc1F ; CN1CC2CCC(C1)N2Cc1ccc(NC(=O)Nc2ccc(Oc3ccnc4[nH]cc(Cl)c34)cc2F)cc1C(F)(F)F ; CN1CCN(Cc2ccc(NC(=O)Nc3ccc(Oc4ccnc5[nH]cc(Cl)c45)cc3F)cc2C(F)(F)F)C2(CC2)C1]

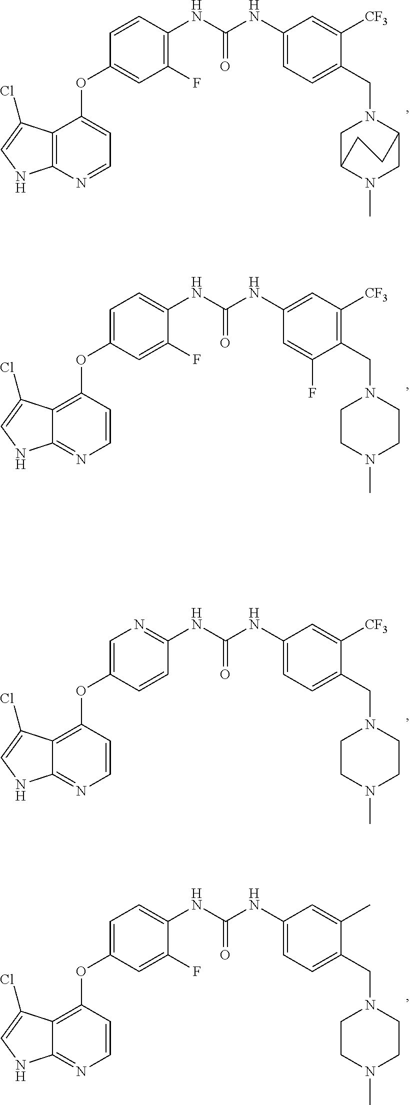 [SMILES: CN1CC2CCC1CN2Cc1ccc(NC(=O)Nc2ccc(Oc3ccnc4[nH]cc(Cl)c34)cc2F)cc1C(F)(F)F ; CN1CCN(Cc2c(F)cc(NC(=O)Nc3ccc(Oc4ccnc5[nH]cc(Cl)c45)cc3F)cc2C(F)(F)F)CC1 ; CN1CCN(Cc2ccc(NC(=O)Nc3ccc(Oc4ccnc5[nH]cc(Cl)c45)cn3)cc2C(F)(F)F)CC1 ; Cc1cc(NC(=O)Nc2ccc(Oc3ccnc4[nH]cc(Cl)c34)cc2F)ccc1CN1CCN(C)CC1]

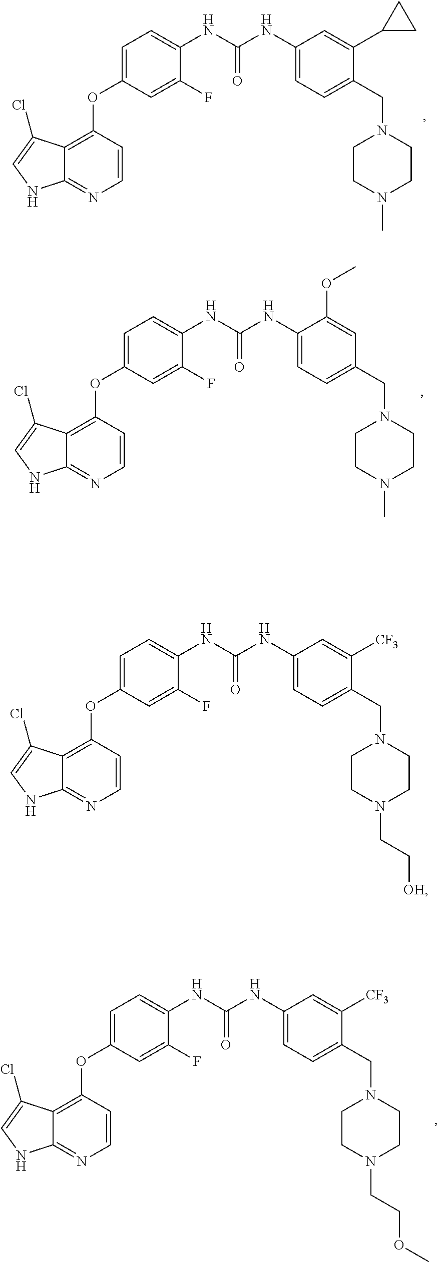

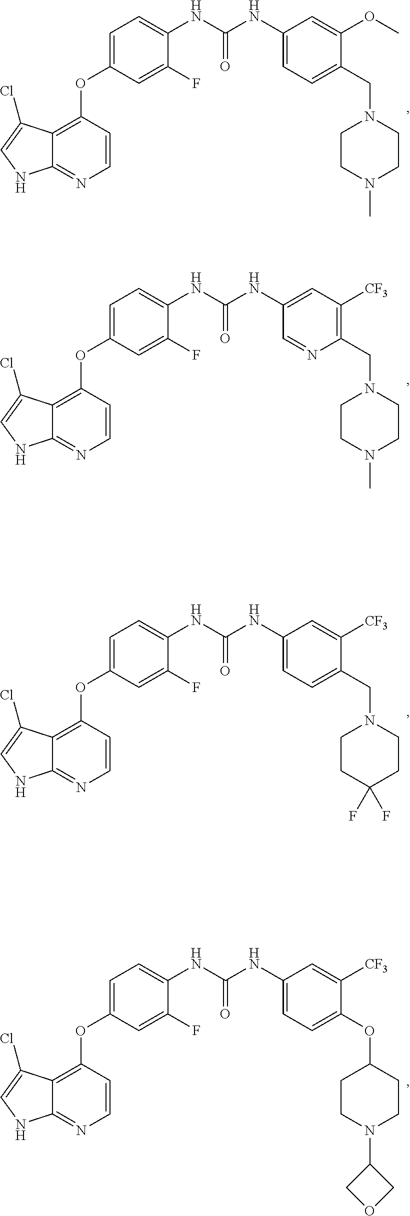 [SMILES: COc1cc(NC(=O)Nc2ccc(Oc3ccnc4[nH]cc(Cl)c34)cc2F)ccc1CN1CCN(C)CC1 ; CN1CCN(Cc2ncc(NC(=O)Nc3ccc(Oc4ccnc5[nH]cc(Cl)c45)cc3F)cc2C(F)(F)F)CC1 ; O=C(Nc1ccc(CN2CCC(F)(F)CC2)c(C(F)(F)F)c1)Nc1ccc(Oc2ccnc3[nH]cc(Cl)c23)cc1F ; O=C(Nc1ccc(OC2CCN(C3COC3)CC2)c(C(F)(F)F)c1)Nc1ccc(Oc2ccnc3[nH]cc(Cl)c23)cc1F]

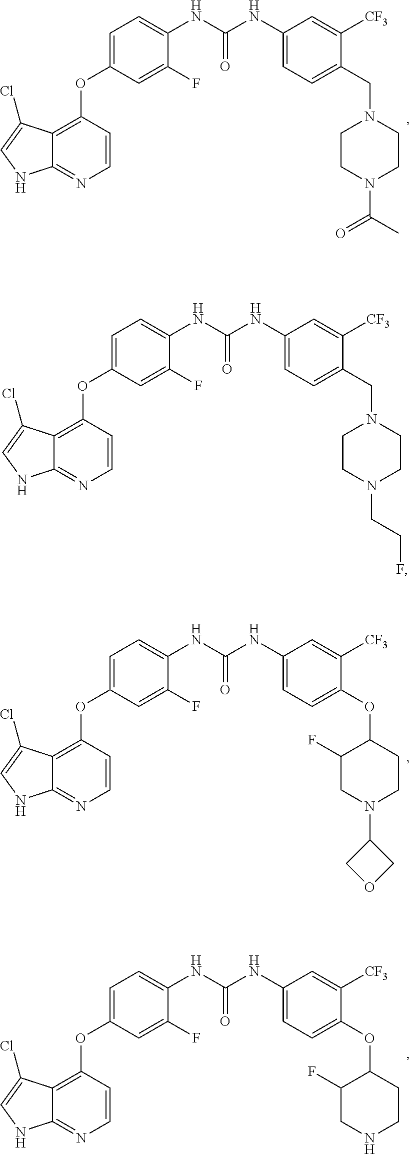 [SMILES: CC(=O)N1CCN(Cc2ccc(NC(=O)Nc3ccc(Oc4ccnc5[nH]cc(Cl)c45)cc3F)cc2C(F)(F)F)CC1 ; O=C(Nc1ccc(CN2CCN(CCF)CC2)c(C(F)(F)F)c1)Nc1ccc(Oc2ccnc3[nH]cc(Cl)c23)cc1F ; O=C(Nc1ccc(OC2CCN(C3COC3)CC2F)c(C(F)(F)F)c1)Nc1ccc(Oc2ccnc3[nH]cc(Cl)c23)cc1F ; O=C(Nc1ccc(OC2CCNCC2F)c(C(F)(F)F)c1)Nc1ccc(Oc2ccnc3[nH]cc(Cl)c23)cc1F]

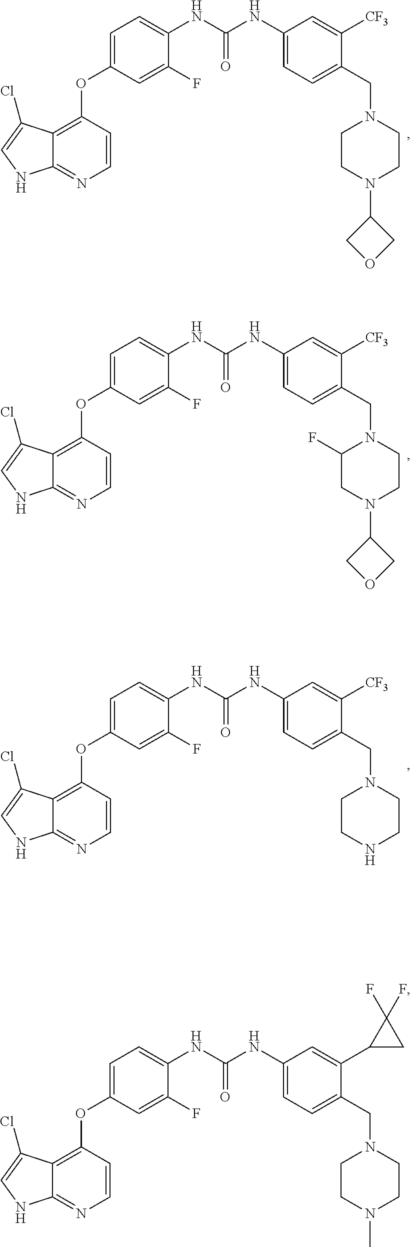 [SMILES: O=C(Nc1ccc(CN2CCN(C3COC3)CC2)c(C(F)(F)F)c1)Nc1ccc(Oc2ccnc3[nH]cc(Cl)c23)cc1F ; O=C(Nc1ccc(CN2CCN(C3COC3)CC2F)c(C(F)(F)F)c1)Nc1ccc(Oc2ccnc3[nH]cc(Cl)c23)cc1F ; O=C(Nc1ccc(CN2CCNCC2)c(C(F)(F)F)c1)Nc1ccc(Oc2ccnc3[nH]cc(Cl)c23)cc1F ; CN1CCN(Cc2ccc(NC(=O)Nc3ccc(Oc4ccnc5[nH]cc(Cl)c45)cc3F)cc2C2CC2(F)F)CC1]

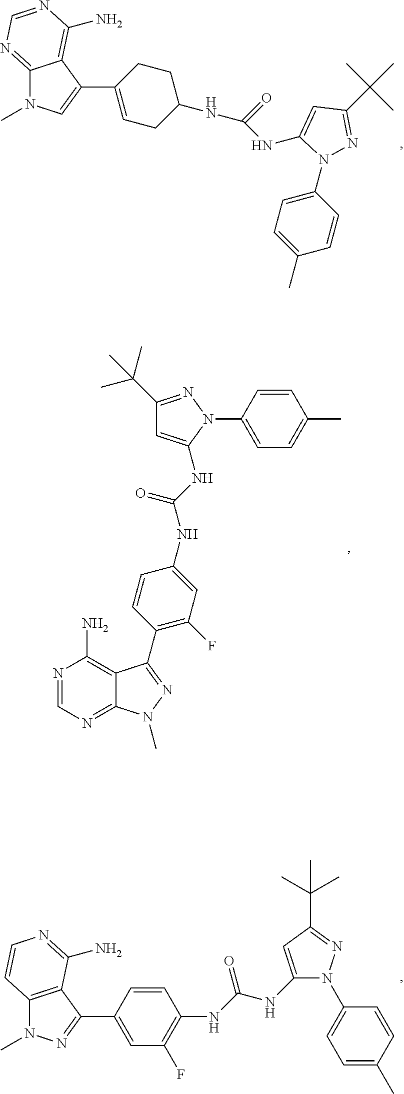

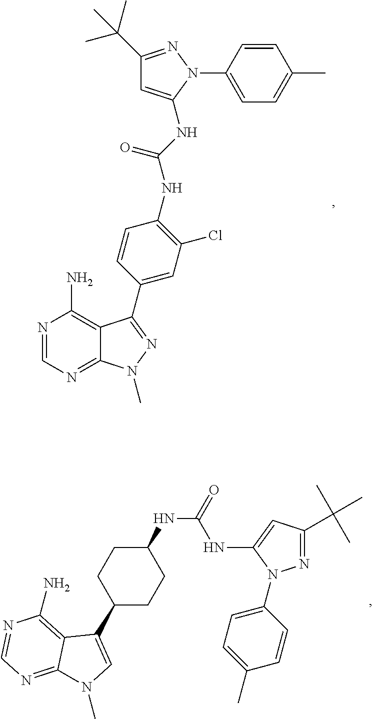 [SMILES: Cc1ccc(-n2nc(C(C)(C)C)cc2NC(=O)Nc2ccc(-c3nn(C)c4ncnc(N)c34)cc2Cl)cc1 ; Cc1ccc(-n2nc(C(C)(C)C)cc2NC(=O)N[C@H]2CC[C@@H](c3cn(C)c4ncnc(N)c43)CC2)cc1]

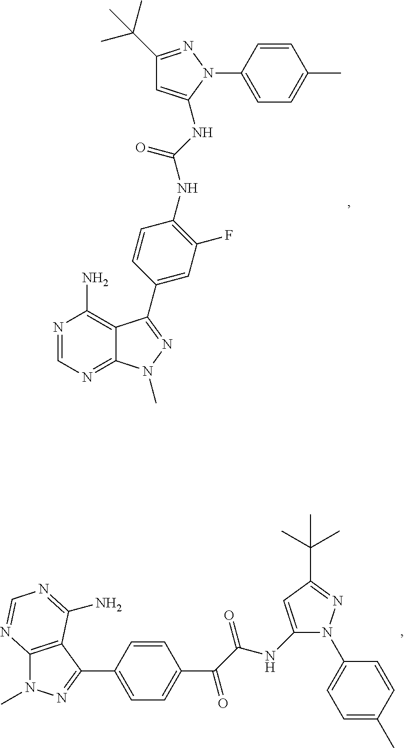

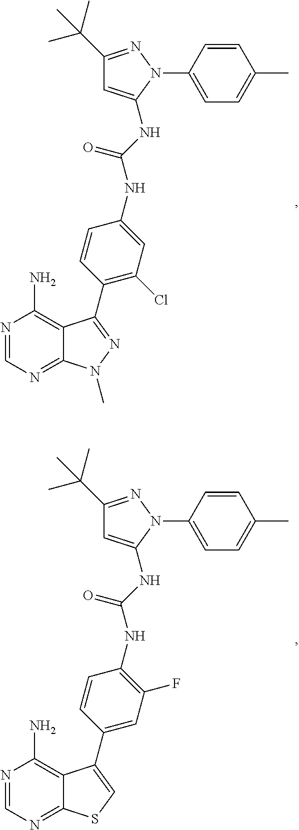 [SMILES: Cc1ccc(-n2nc(C(C)(C)C)cc2NC(=O)Nc2ccc(-c3nn(C)c4ncnc(N)c34)c(Cl)c2)cc1 ; Cc1ccc(-n2nc(C(C)(C)C)cc2NC(=O)Nc2ccc(-c3csc4ncnc(N)c34)cc2F)cc1]

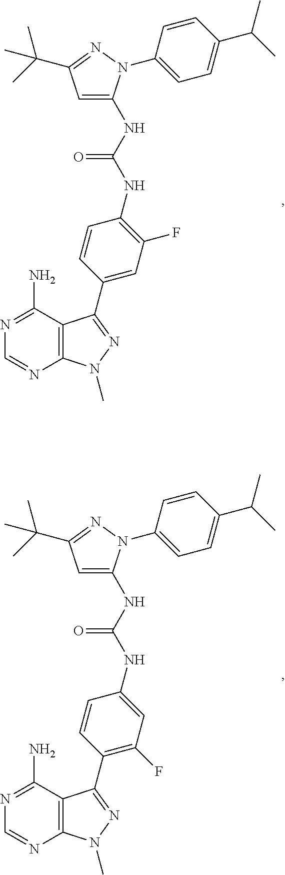

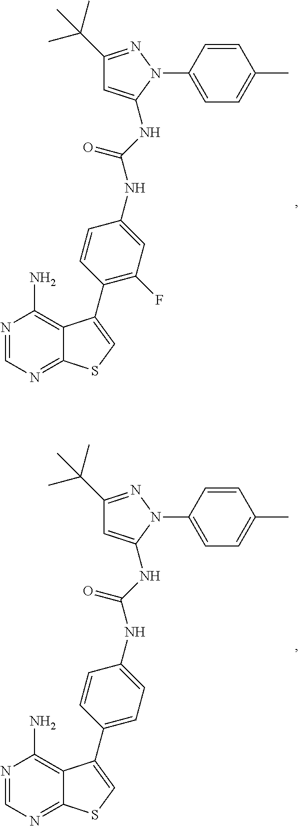 [SMILES: Cc1ccc(-n2nc(C(C)(C)C)cc2NC(=O)Nc2ccc(-c3csc4ncnc(N)c34)c(F)c2)cc1 ; Cc1ccc(-n2nc(C(C)(C)C)cc2NC(=O)Nc2ccc(-c3csc4ncnc(N)c34)cc2)cc1]

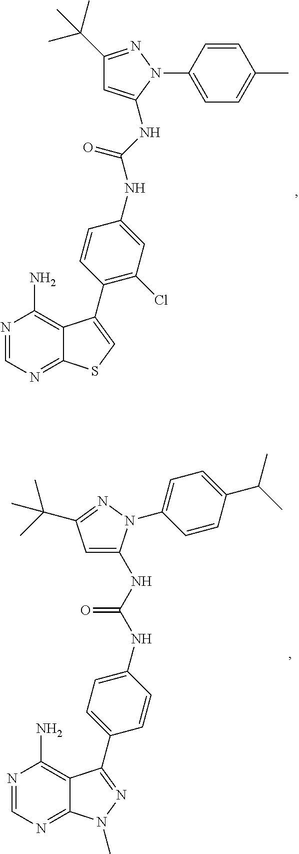

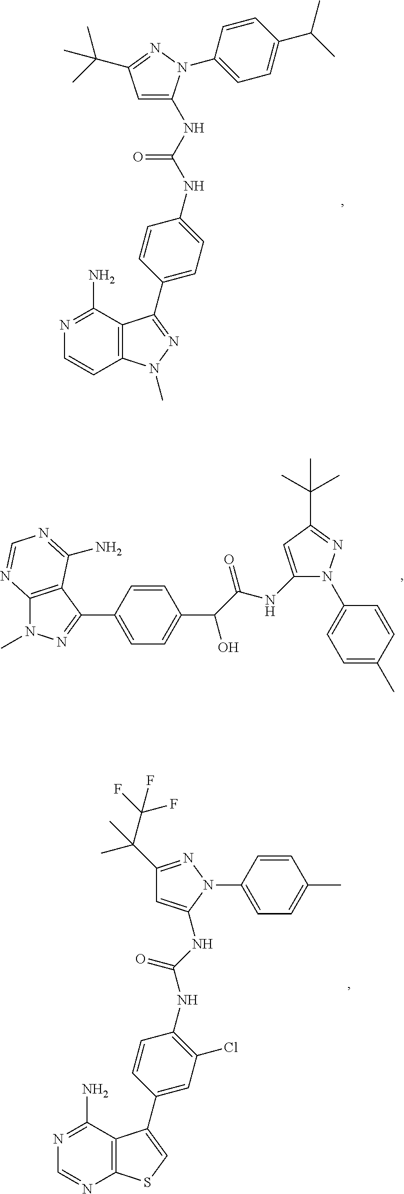

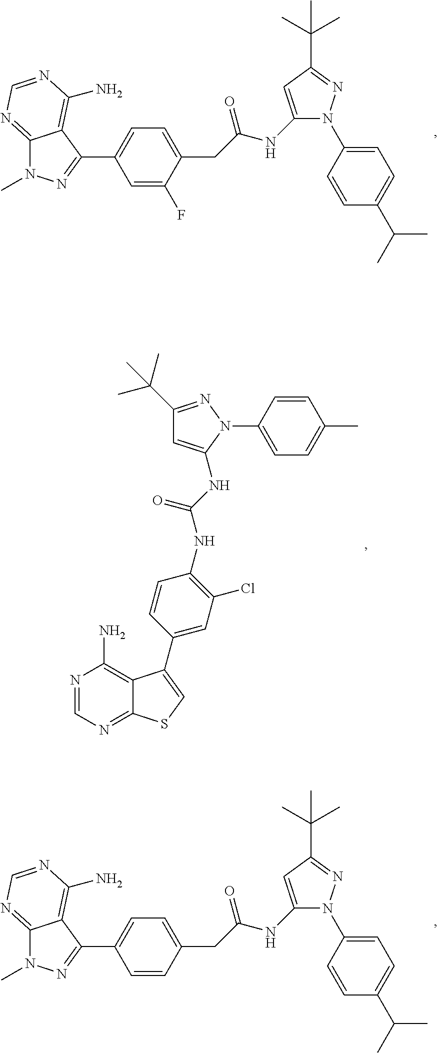

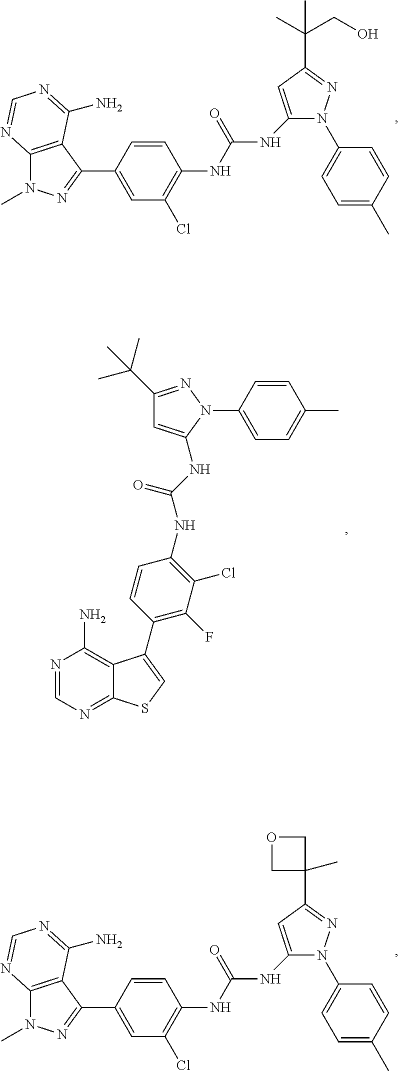

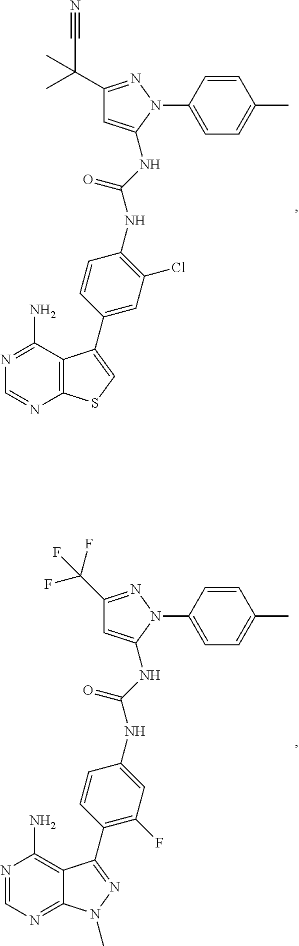 [SMILES: Cc1ccc(-n2nc(C(C)(C)C#N)cc2NC(=O)Nc2ccc(-c3csc4ncnc(N)c34)cc2Cl)cc1 ; Cc1ccc(-n2nc(C(F)(F)F)cc2NC(=O)Nc2ccc(-c3nn(C)c4ncnc(N)c34)c(F)c2)cc1]

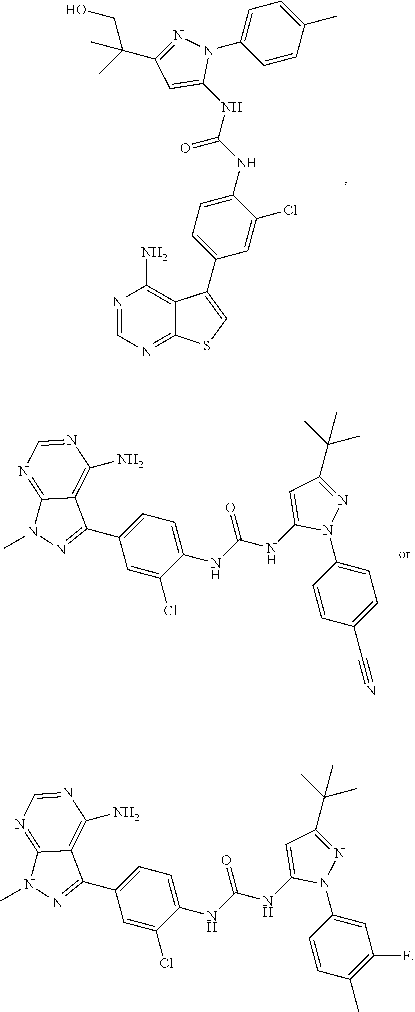

116. A composition comprising at least one NEK7 protein and a NEK7 small molecule inhibitor compound comprising at least one of the following features:
i. a hinge-binding element comprising at least one hydrogen donor and at least one hydrogen acceptor;
ii. a flexible linker;
iii. a urea-type linker; or
iv. a hydrophobic back pocket group.

117-229. (canceled)

230. The composition of claim 116, wherein the NEK7 small molecule inhibitor compound has one of the following structures, or a pharmaceutically acceptable salt, stereoisomer, or prodrug thereof:

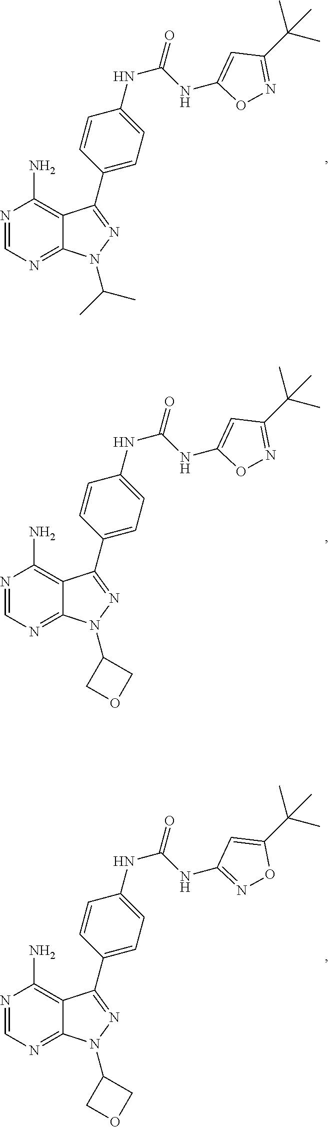 [SMILES: CC(C)n1nc(-c2ccc(NC(=O)Nc3cc(C(C)(C)C)no3)cc2)c2c(N)ncnc21 ; CC(C)(C)c1cc(NC(=O)Nc2ccc(-c3nn(C4COC4)c4ncnc(N)c34)cc2)on1 ; CC(C)(C)c1cc(NC(=O)Nc2ccc(-c3nn(C4COC4)c4ncnc(N)c34)cc2)no1]

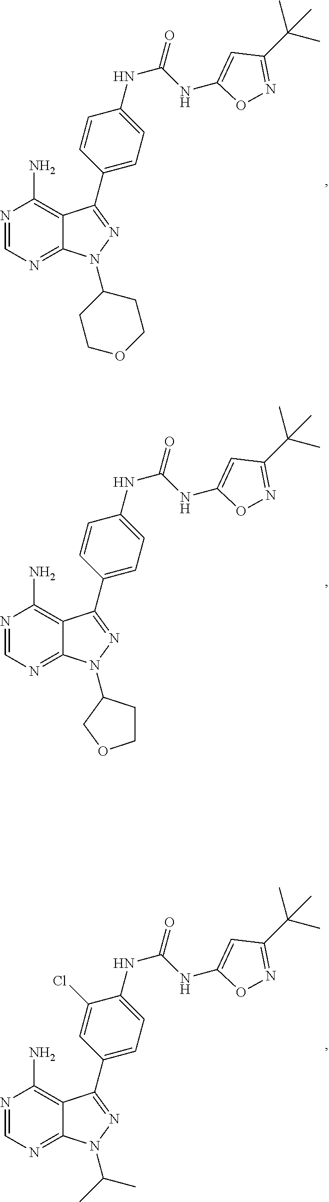 [SMILES: CC(C)(C)c1cc(NC(=O)Nc2ccc(-c3nn(C4CCOCC4)c4ncnc(N)c34)cc2)on1 ; CC(C)(C)c1cc(NC(=O)Nc2ccc(-c3nn(C4CCOC4)c4ncnc(N)c34)cc2)on1 ; CC(C)n1nc(-c2ccc(NC(=O)Nc3cc(C(C)(C)C)no3)c(Cl)c2)c2c(N)ncnc21]

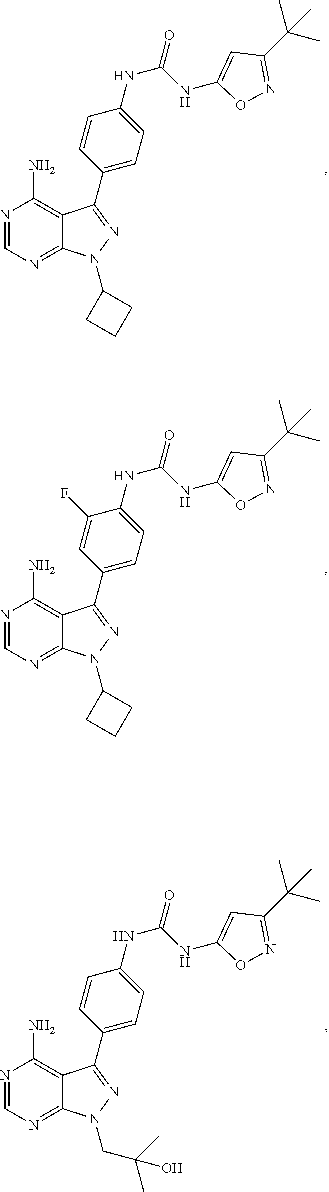 [SMILES: CC(C)(C)c1cc(NC(=O)Nc2ccc(-c3nn(C4CCC4)c4ncnc(N)c34)cc2)on1 ; CC(C)(C)c1cc(NC(=O)Nc2ccc(-c3nn(C4CCC4)c4ncnc(N)c34)cc2F)on1 ; CC(C)(O)Cn1nc(-c2ccc(NC(=O)Nc3cc(C(C)(C)C)no3)cc2)c2c(N)ncnc21]

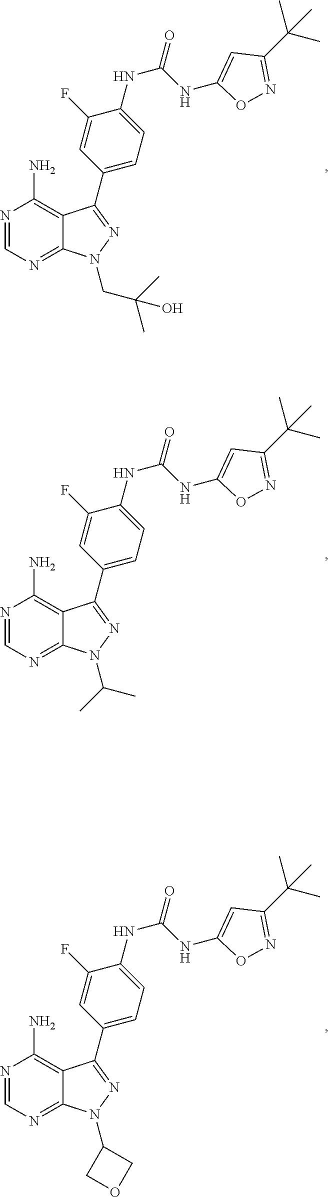 [SMILES: CC(C)(O)Cn1nc(-c2ccc(NC(=O)Nc3cc(C(C)(C)C)no3)c(F)c2)c2c(N)ncnc21 ; CC(C)n1nc(-c2ccc(NC(=O)Nc3cc(C(C)(C)C)no3)c(F)c2)c2c(N)ncnc21 ; CC(C)(C)c1cc(NC(=O)Nc2ccc(-c3nn(C4COC4)c4ncnc(N)c34)cc2F)on1]

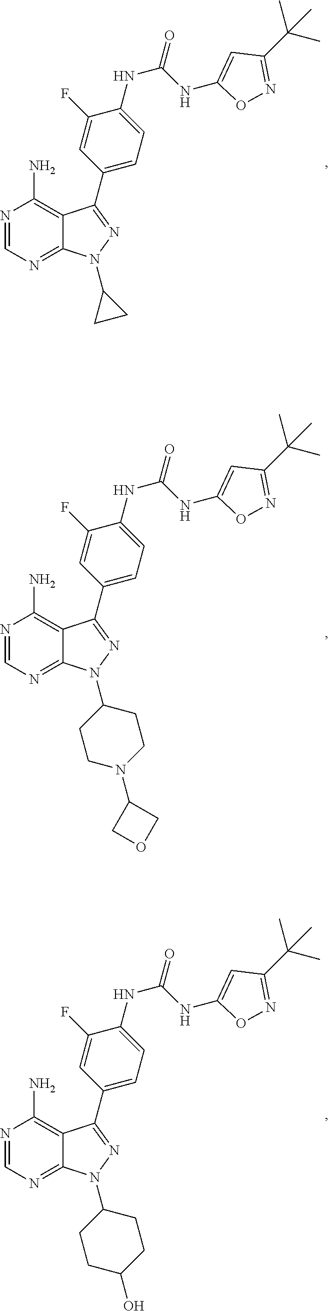 [SMILES: CC(C)(C)c1cc(NC(=O)Nc2ccc(-c3nn(C4CC4)c4ncnc(N)c34)cc2F)on1 ; CC(C)(C)c1cc(NC(=O)Nc2ccc(-c3nn(C4CCN(C5COC5)CC4)c4ncnc(N)c34)cc2F)on1 ; CC(C)(C)c1cc(NC(=O)Nc2ccc(-c3nn(C4CCC(O)CC4)c4ncnc(N)c34)cc2F)on1]

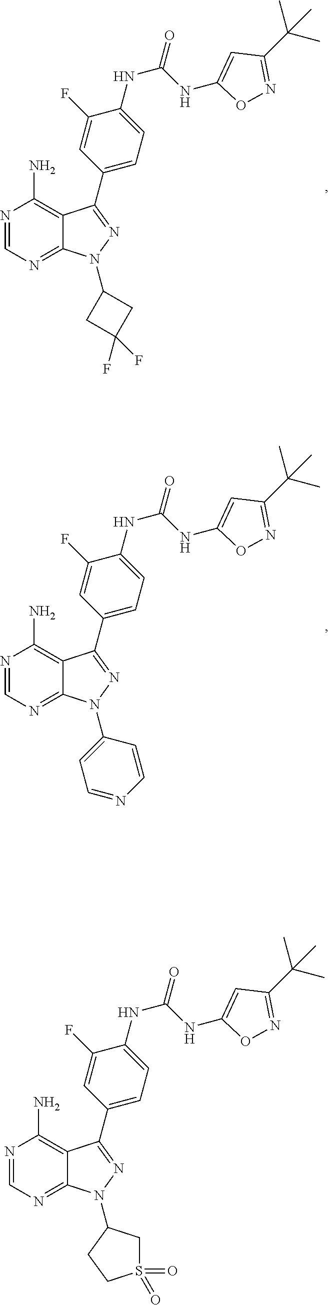 [SMILES: CC(C)(C)c1cc(NC(=O)Nc2ccc(-c3nn(C4CC(F)(F)C4)c4ncnc(N)c34)cc2F)on1 ; CC(C)(C)c1cc(NC(=O)Nc2ccc(-c3nn(-c4ccncc4)c4ncnc(N)c34)cc2F)on1 ; CC(C)(C)c1cc(NC(=O)Nc2ccc(-c3nn(C4CCS(=O)(=O)C4)c4ncnc(N)c34)cc2F)on1]

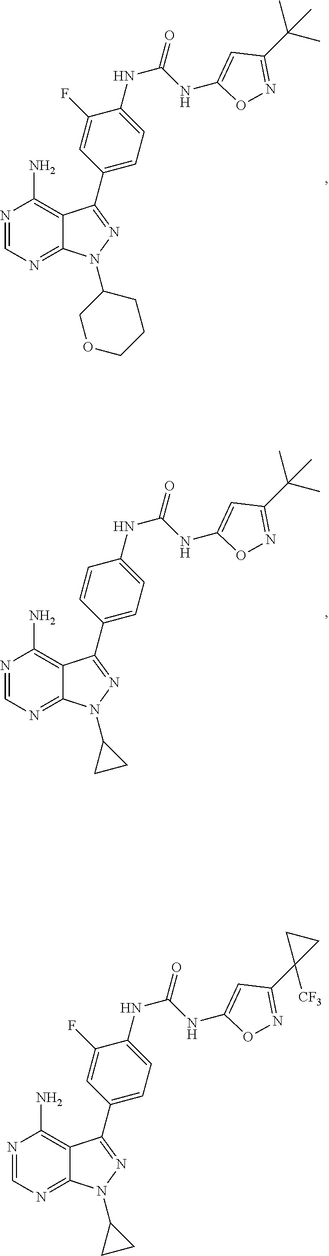 [SMILES: CC(C)(C)c1cc(NC(=O)Nc2ccc(-c3nn(C4CCCOC4)c4ncnc(N)c34)cc2F)on1 ; CC(C)(C)c1cc(NC(=O)Nc2ccc(-c3nn(C4CC4)c4ncnc(N)c34)cc2)on1 ; Nc1ncnc2c1c(-c1ccc(NC(=O)Nc3cc(C4(C(F)(F)F)CC4)no3)c(F)c1)nn2C1CC1]

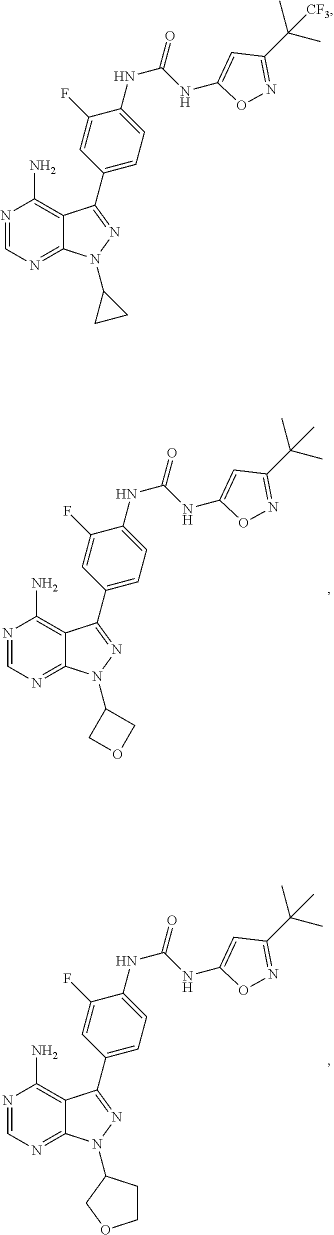 [SMILES: CC(C)(c1cc(NC(=O)Nc2ccc(-c3nn(C4CC4)c4ncnc(N)c34)cc2F)on1)C(F)(F)F ; CC(C)(C)c1cc(NC(=O)Nc2ccc(-c3nn(C4COC4)c4ncnc(N)c34)cc2F)on1 ; CC(C)(C)c1cc(NC(=O)Nc2ccc(-c3nn(C4CCOC4)c4ncnc(N)c34)cc2F)on1]

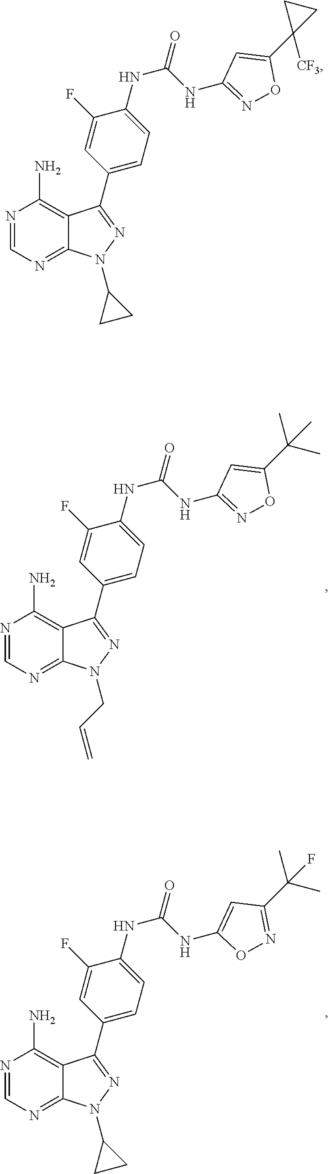 [SMILES: Nc1ncnc2c1c(-c1ccc(NC(=O)Nc3cc(C4(C(F)(F)F)CC4)on3)c(F)c1)nn2C1CC1 ; C=CCn1nc(-c2ccc(NC(=O)Nc3cc(C(C)(C)C)on3)c(F)c2)c2c(N)ncnc21 ; CC(C)(F)c1cc(NC(=O)Nc2ccc(-c3nn(C4CC4)c4ncnc(N)c34)cc2F)on1]

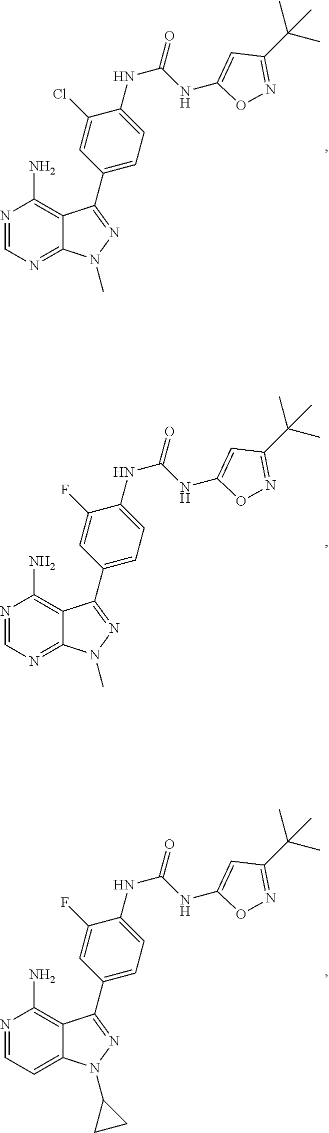 [SMILES: Cn1nc(-c2ccc(NC(=O)Nc3cc(C(C)(C)C)no3)c(Cl)c2)c2c(N)ncnc21 ; Cn1nc(-c2ccc(NC(=O)Nc3cc(C(C)(C)C)no3)c(F)c2)c2c(N)ncnc21 ; CC(C)(C)c1cc(NC(=O)Nc2ccc(-c3nn(C4CC4)c4ccnc(N)c34)cc2F)on1]

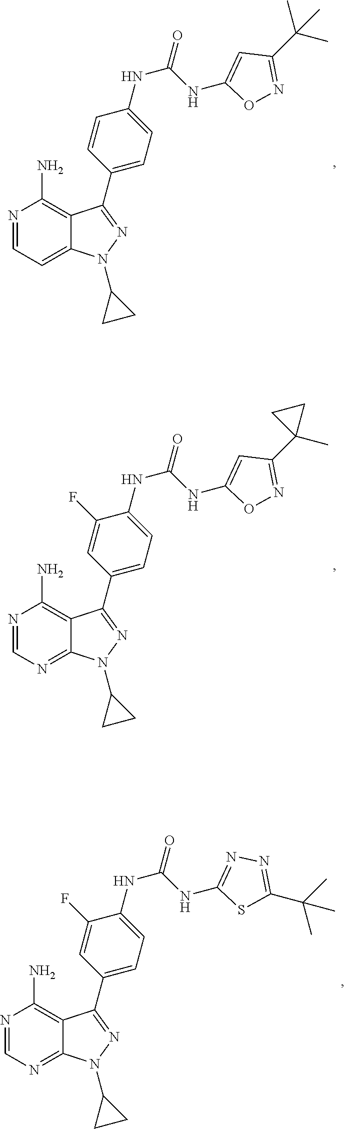 [SMILES: CC(C)(C)c1cc(NC(=O)Nc2ccc(-c3nn(C4CC4)c4ccnc(N)c34)cc2)on1 ; CC1(c2cc(NC(=O)Nc3ccc(-c4nn(C5CC5)c5ncnc(N)c45)cc3F)on2)CC1 ; CC(C)(C)c1nnc(NC(=O)Nc2ccc(-c3nn(C4CC4)c4ncnc(N)c34)cc2F)s1]

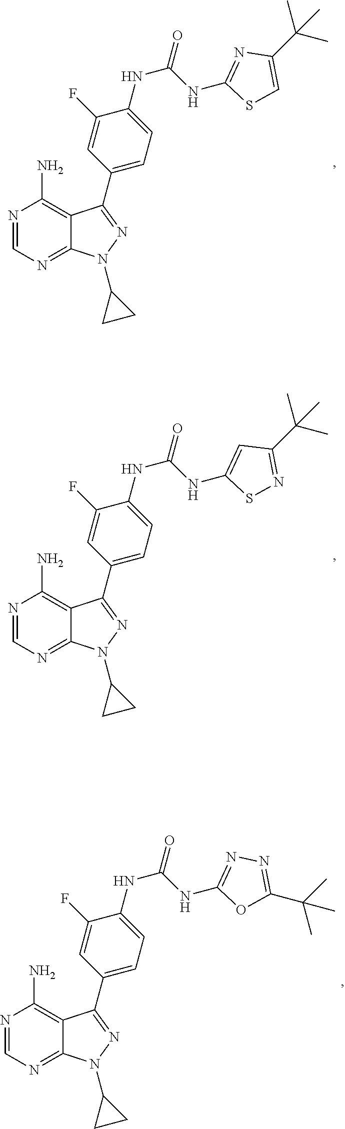 [SMILES: CC(C)(C)c1csc(NC(=O)Nc2ccc(-c3nn(C4CC4)c4ncnc(N)c34)cc2F)n1 ; CC(C)(C)c1cc(NC(=O)Nc2ccc(-c3nn(C4CC4)c4ncnc(N)c34)cc2F)sn1 ; CC(C)(C)c1nnc(NC(=O)Nc2ccc(-c3nn(C4CC4)c4ncnc(N)c34)cc2F)o1]

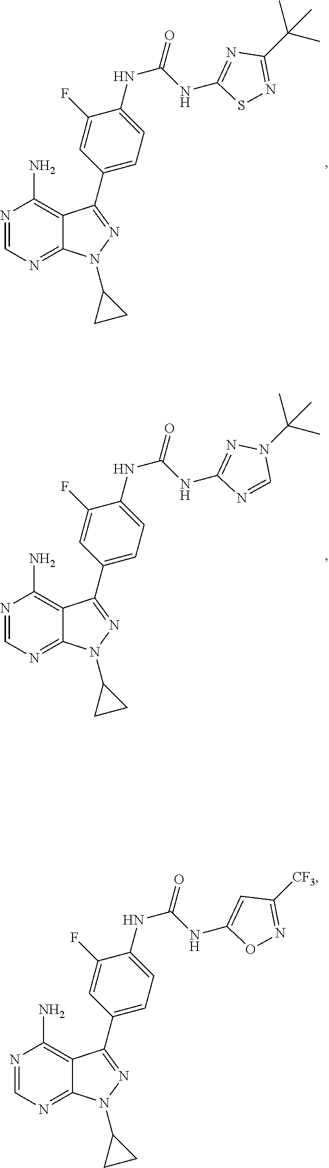 [SMILES: CC(C)(C)c1nsc(NC(=O)Nc2ccc(-c3nn(C4CC4)c4ncnc(N)c34)cc2F)n1 ; CC(C)(C)n1cnc(NC(=O)Nc2ccc(-c3nn(C4CC4)c4ncnc(N)c34)cc2F)n1 ; Nc1ncnc2c1c(-c1ccc(NC(=O)Nc3cc(C(F)(F)F)no3)c(F)c1)nn2C1CC1]

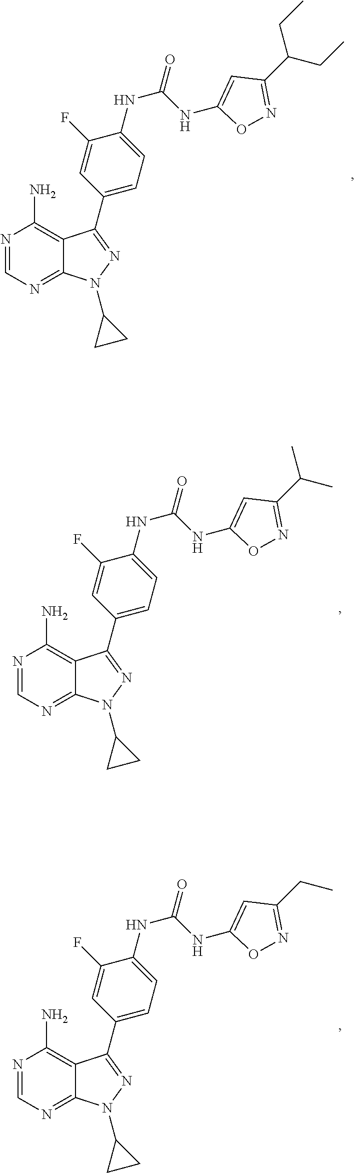 [SMILES: CCC(CC)c1cc(NC(=O)Nc2ccc(-c3nn(C4CC4)c4ncnc(N)c34)cc2F)on1 ; CC(C)c1cc(NC(=O)Nc2ccc(-c3nn(C4CC4)c4ncnc(N)c34)cc2F)on1 ; CCc1cc(NC(=O)Nc2ccc(-c3nn(C4CC4)c4ncnc(N)c34)cc2F)on1]

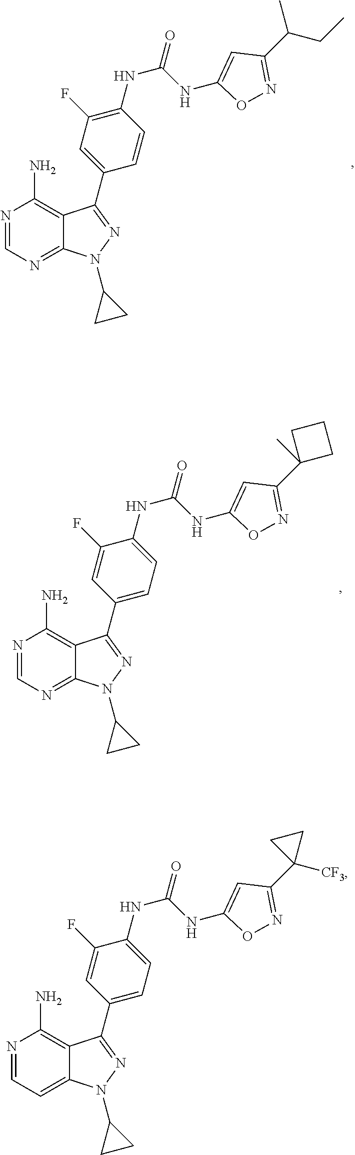 [SMILES: CCC(C)c1cc(NC(=O)Nc2ccc(-c3nn(C4CC4)c4ncnc(N)c34)cc2F)on1 ; CC1(c2cc(NC(=O)Nc3ccc(-c4nn(C5CC5)c5ncnc(N)c45)cc3F)on2)CCC1 ; Nc1nccc2c1c(-c1ccc(NC(=O)Nc3cc(C4(C(F)(F)F)CC4)no3)c(F)c1)nn2C1CC1]

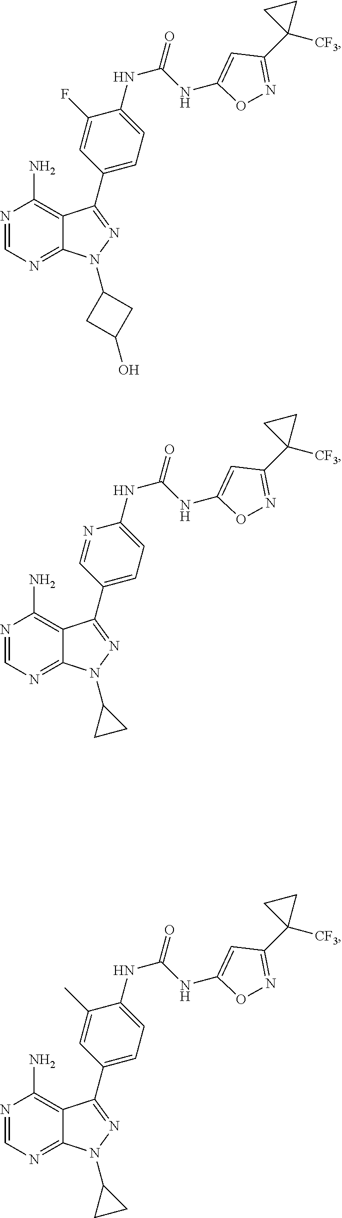 [SMILES: Nc1ncnc2c1c(-c1ccc(NC(=O)Nc3cc(C4(C(F)(F)F)CC4)no3)c(F)c1)nn2C1CC(O)C1 ; Nc1ncnc2c1c(-c1ccc(NC(=O)Nc3cc(C4(C(F)(F)F)CC4)no3)nc1)nn2C1CC1 ; Cc1cc(-c2nn(C3CC3)c3ncnc(N)c23)ccc1NC(=O)Nc1cc(C2(C(F)(F)F)CC2)no1]

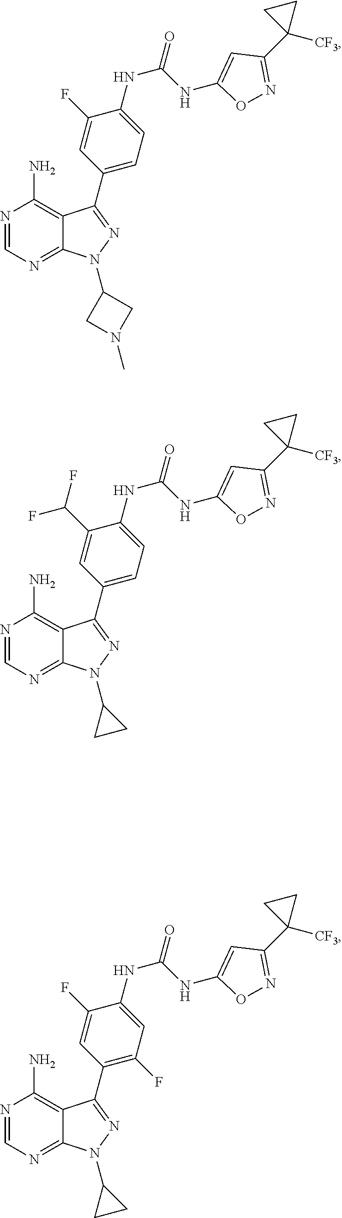 [SMILES: CN1CC(n2nc(-c3ccc(NC(=O)Nc4cc(C5(C(F)(F)F)CC5)no4)c(F)c3)c3c(N)ncnc32)C1 ; Nc1ncnc2c1c(-c1ccc(NC(=O)Nc3cc(C4(C(F)(F)F)CC4)no3)c(C(F)F)c1)nn2C1CC1 ; Nc1ncnc2c1c(-c1cc(F)c(NC(=O)Nc3cc(C4(C(F)(F)F)CC4)no3)cc1F)nn2C1CC1]

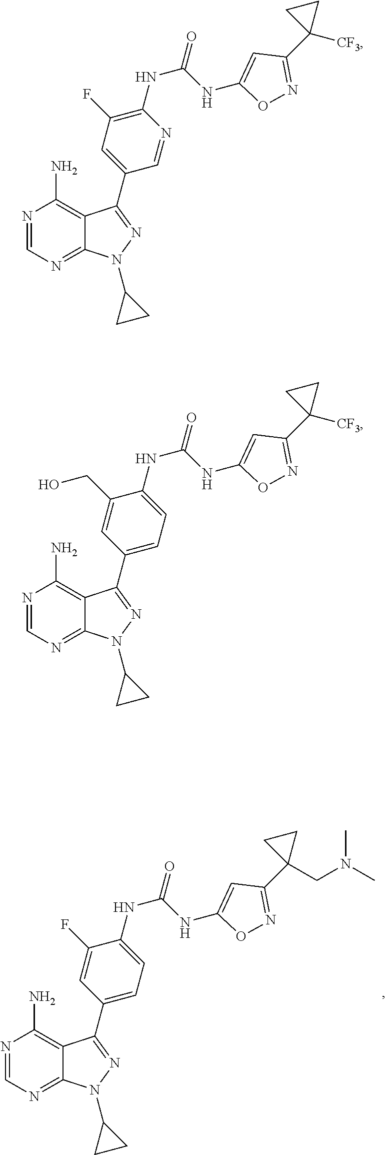 [SMILES: Nc1ncnc2c1c(-c1cnc(NC(=O)Nc3cc(C4(C(F)(F)F)CC4)no3)c(F)c1)nn2C1CC1 ; Nc1ncnc2c1c(-c1ccc(NC(=O)Nc3cc(C4(C(F)(F)F)CC4)no3)c(CO)c1)nn2C1CC1 ; CN(C)CC1(c2cc(NC(=O)Nc3ccc(-c4nn(C5CC5)c5ncnc(N)c45)cc3F)on2)CC1]

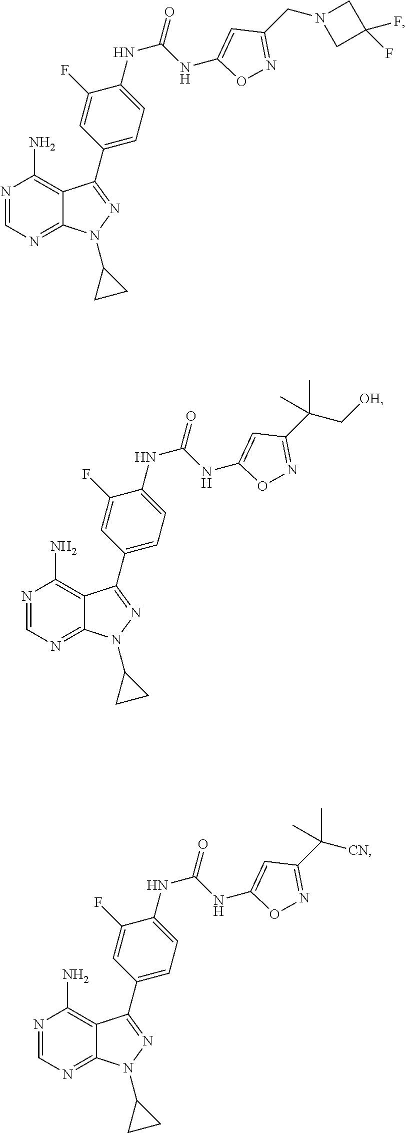 [SMILES: Nc1ncnc2c1c(-c1ccc(NC(=O)Nc3cc(CN4CC(F)(F)C4)no3)c(F)c1)nn2C1CC1 ; CC(C)(CO)c1cc(NC(=O)Nc2ccc(-c3nn(C4CC4)c4ncnc(N)c34)cc2F)on1 ; CC(C)(C#N)c1cc(NC(=O)Nc2ccc(-c3nn(C4CC4)c4ncnc(N)c34)cc2F)on1]

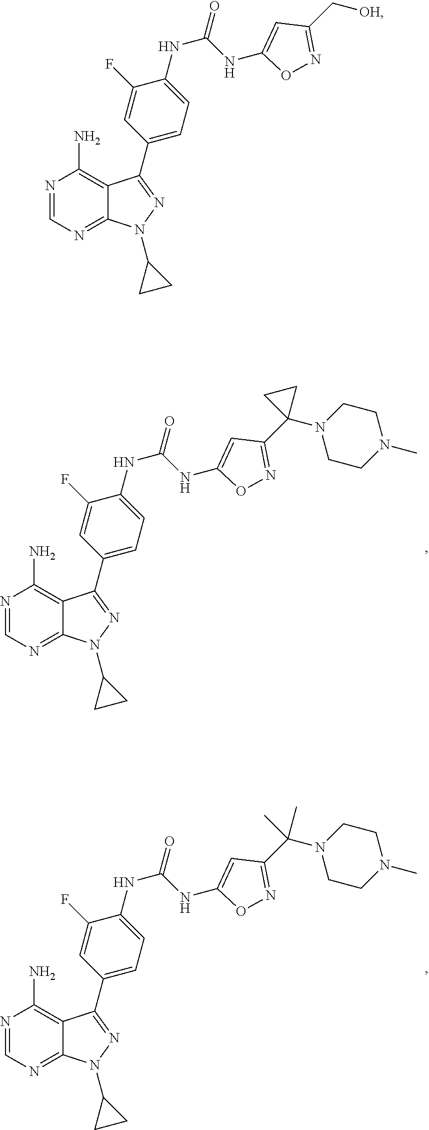 [SMILES: Nc1ncnc2c1c(-c1ccc(NC(=O)Nc3cc(CO)no3)c(F)c1)nn2C1CC1 ; CN1CCN(C2(c3cc(NC(=O)Nc4ccc(-c5nn(C6CC6)c6ncnc(N)c56)cc4F)on3)CC2)CC1 ; CN1CCN(C(C)(C)c2cc(NC(=O)Nc3ccc(-c4nn(C5CC5)c5ncnc(N)c45)cc3F)on2)CC1]

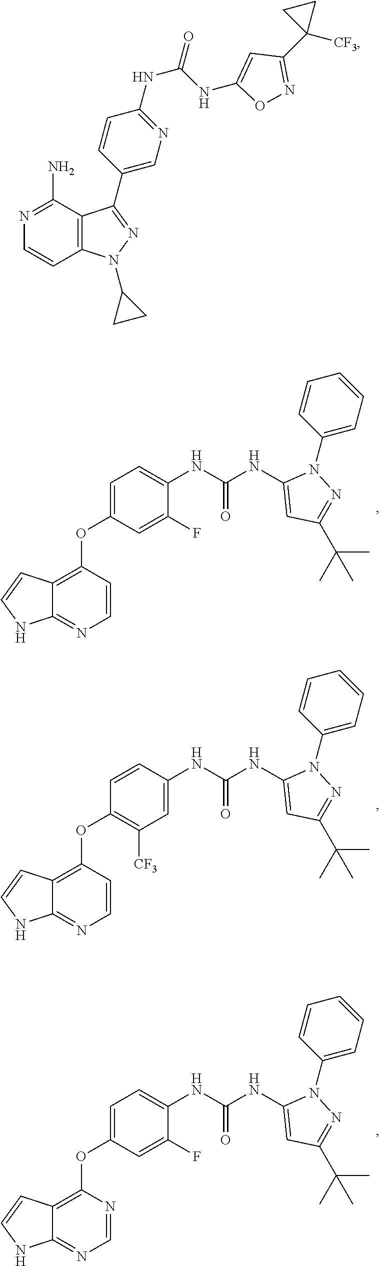 [SMILES: Nc1nccc2c1c(-c1ccc(NC(=O)Nc3cc(C4(C(F)(F)F)CC4)no3)nc1)nn2C1CC1 ; CC(C)(C)c1cc(NC(=O)Nc2ccc(Oc3ccnc4[nH]ccc34)cc2F)n(-c2ccccc2)n1 ; CC(C)(C)c1cc(NC(=O)Nc2ccc(Oc3ccnc4[nH]ccc34)c(C(F)(F)F)c2)n(-c2ccccc2)n1 ; CC(C)(C)c1cc(NC(=O)Nc2ccc(Oc3ncnc4[nH]ccc34)cc2F)n(-c2ccccc2)n1]

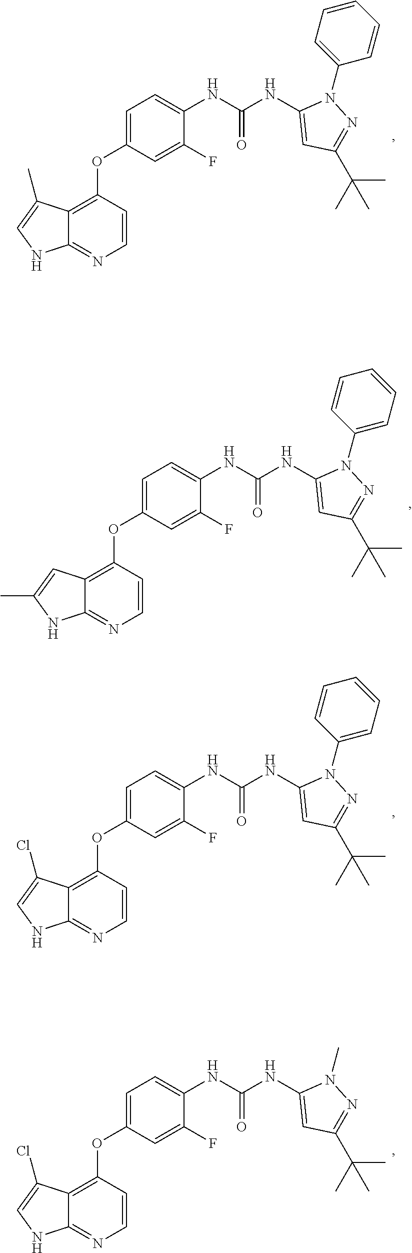

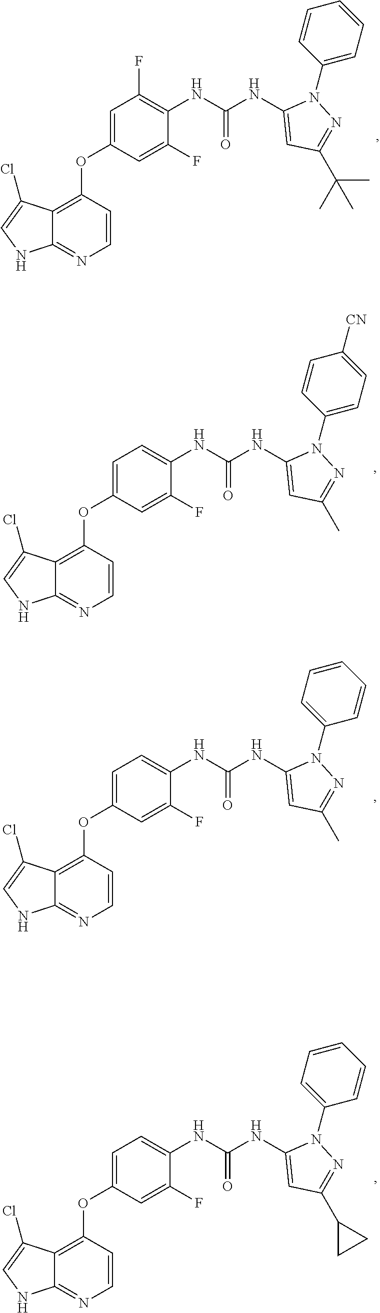 [SMILES: CC(C)(C)c1cc(NC(=O)Nc2c(F)cc(Oc3ccnc4[nH]cc(Cl)c34)cc2F)n(-c2ccccc2)n1 ; Cc1cc(NC(=O)Nc2ccc(Oc3ccnc4[nH]cc(Cl)c34)cc2F)n(-c2ccc(C#N)cc2)n1 ; Cc1cc(NC(=O)Nc2ccc(Oc3ccnc4[nH]cc(Cl)c34)cc2F)n(-c2ccccc2)n1 ; O=C(Nc1ccc(Oc2ccnc3[nH]cc(Cl)c23)cc1F)Nc1cc(C2CC2)nn1-c1ccccc1]

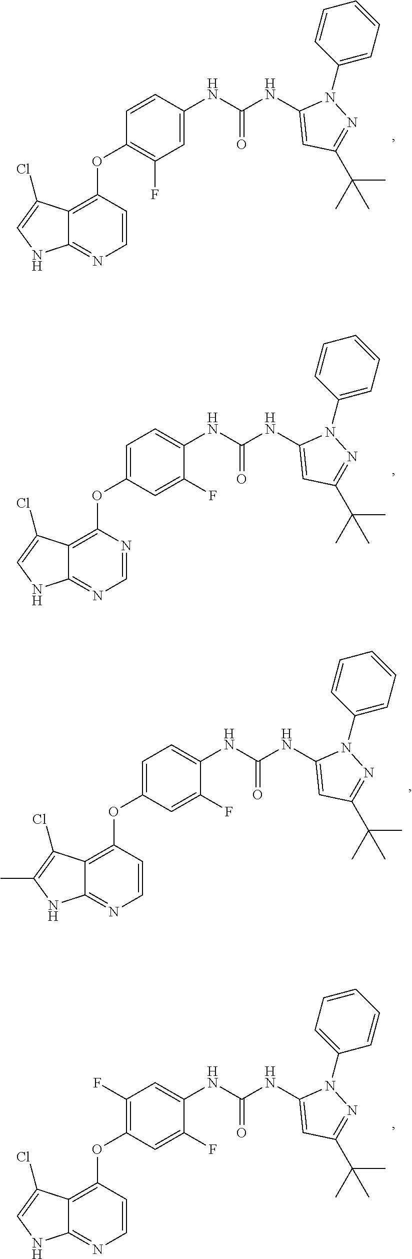

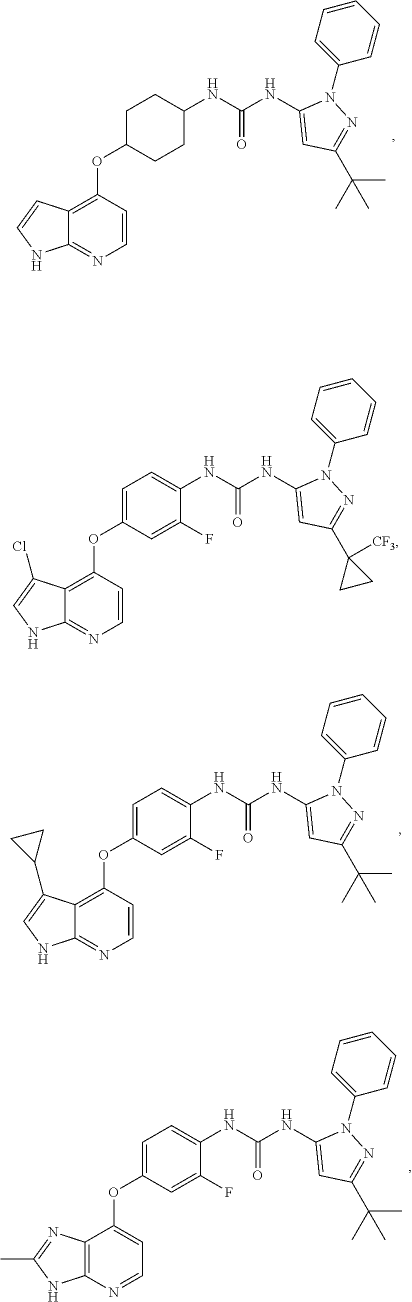

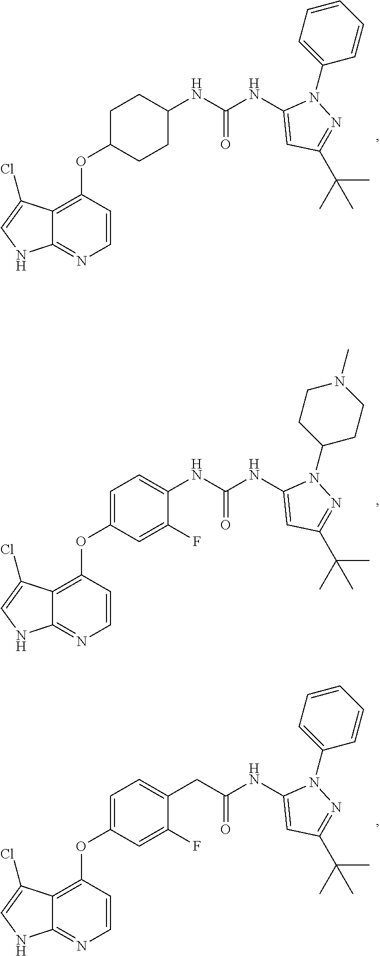 [SMILES: CC(C)(C)c1cc(NC(=O)NC2CCC(Oc3ccnc4[nH]cc(Cl)c34)CC2)n(-c2ccccc2)n1 ; CN1CCC(n2nc(C(C)(C)C)cc2NC(=O)Nc2ccc(Oc3ccnc4[nH]cc(Cl)c34)cc2F)CC1 ; CC(C)(C)c1cc(NC(=O)Cc2ccc(Oc3ccnc4[nH]cc(Cl)c34)cc2F)n(-c2ccccc2)n1]

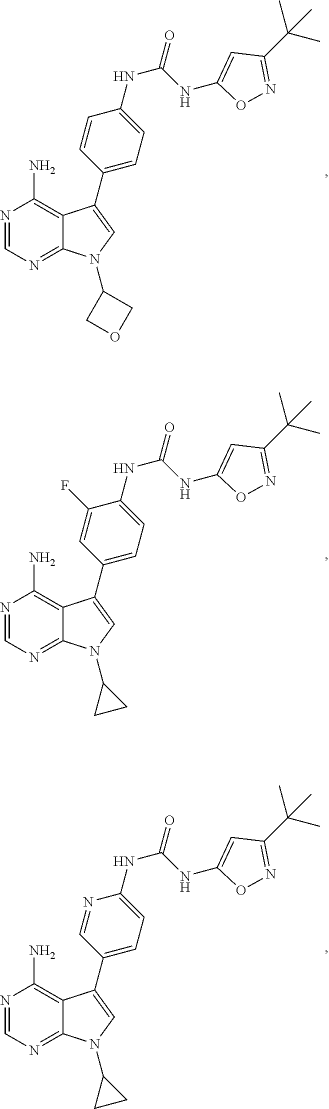 [SMILES: CC(C)(C)c1cc(NC(=O)Nc2ccc(-c3cn(C4COC4)c4ncnc(N)c34)cc2)on1 ; CC(C)(C)c1cc(NC(=O)Nc2ccc(-c3cn(C4CC4)c4ncnc(N)c34)cc2F)on1 ; CC(C)(C)c1cc(NC(=O)Nc2ccc(-c3cn(C4CC4)c4ncnc(N)c34)cn2)on1]

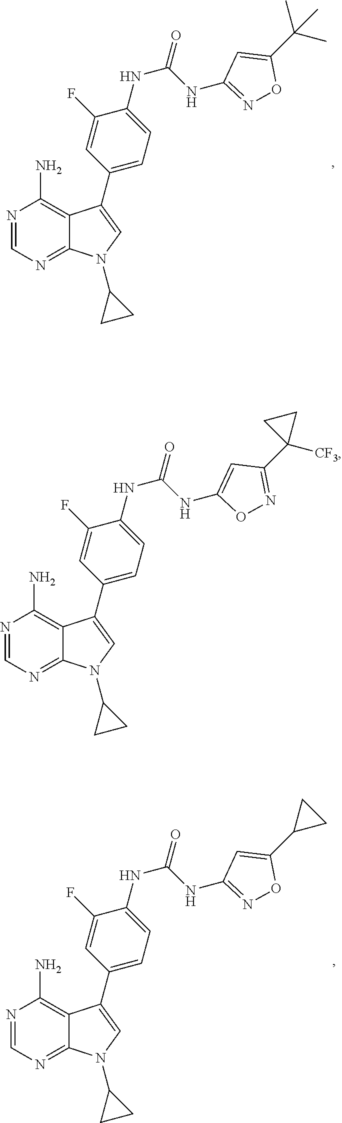 [SMILES: CC(C)(C)c1cc(NC(=O)Nc2ccc(-c3cn(C4CC4)c4ncnc(N)c34)cc2F)no1 ; Nc1ncnc2c1c(-c1ccc(NC(=O)Nc3cc(C4(C(F)(F)F)CC4)no3)c(F)c1)cn2C1CC1 ; Nc1ncnc2c1c(-c1ccc(NC(=O)Nc3cc(C4CC4)on3)c(F)c1)cn2C1CC1]

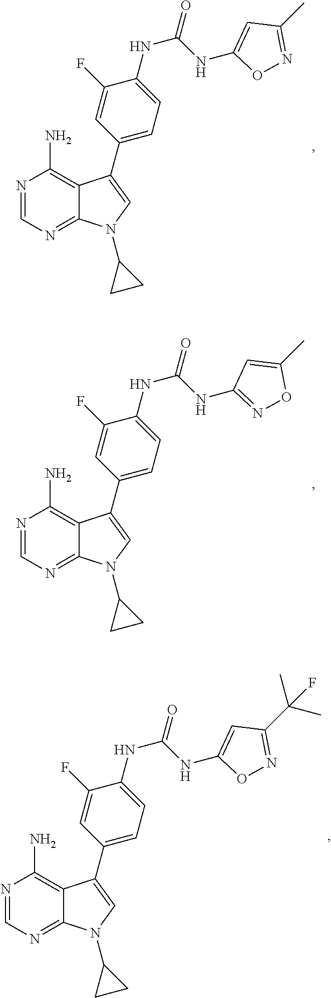 [SMILES: Cc1cc(NC(=O)Nc2ccc(-c3cn(C4CC4)c4ncnc(N)c34)cc2F)on1 ; Cc1cc(NC(=O)Nc2ccc(-c3cn(C4CC4)c4ncnc(N)c34)cc2F)no1 ; CC(C)(F)c1cc(NC(=O)Nc2ccc(-c3cn(C4CC4)c4ncnc(N)c34)cc2F)on1]

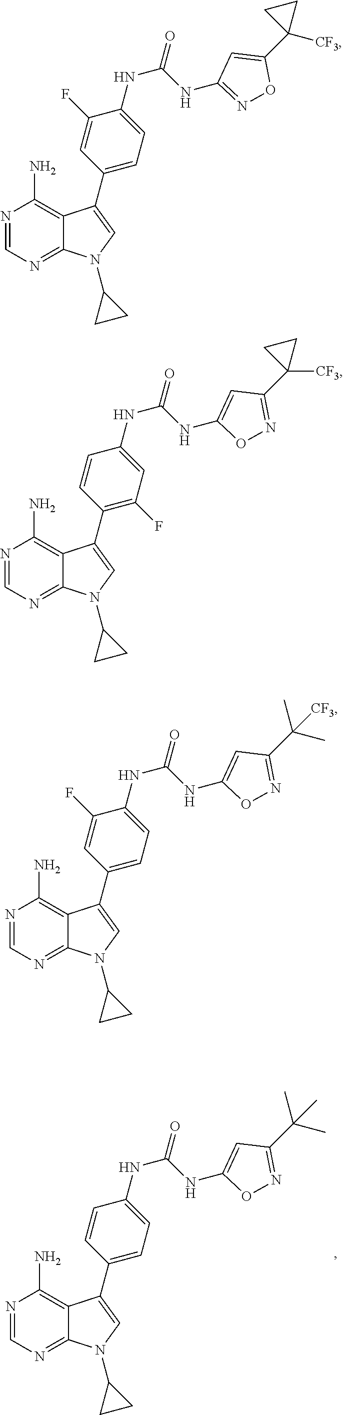 [SMILES: Nc1ncnc2c1c(-c1ccc(NC(=O)Nc3cc(C4(C(F)(F)F)CC4)on3)c(F)c1)cn2C1CC1 ; Nc1ncnc2c1c(-c1ccc(NC(=O)Nc3cc(C4(C(F)(F)F)CC4)no3)cc1F)cn2C1CC1 ; CC(C)(c1cc(NC(=O)Nc2ccc(-c3cn(C4CC4)c4ncnc(N)c34)cc2F)on1)C(F)(F)F ; CC(C)(C)c1cc(NC(=O)Nc2ccc(-c3cn(C4CC4)c4ncnc(N)c34)cc2)on1]

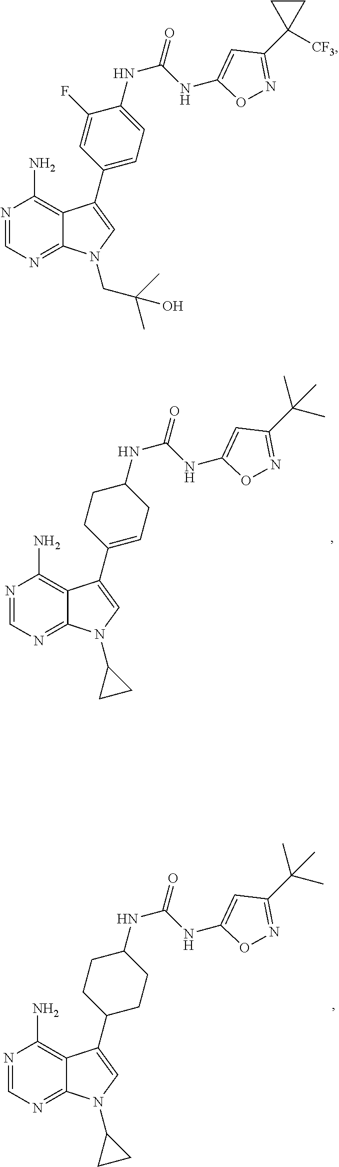 [SMILES: CC(C)(O)Cn1cc(-c2ccc(NC(=O)Nc3cc(C4(C(F)(F)F)CC4)no3)c(F)c2)c2c(N)ncnc21 ; CC(C)(C)c1cc(NC(=O)NC2CC=C(c3cn(C4CC4)c4ncnc(N)c34)CC2)on1 ; CC(C)(C)c1cc(NC(=O)NC2CCC(c3cn(C4CC4)c4ncnc(N)c34)CC2)on1]

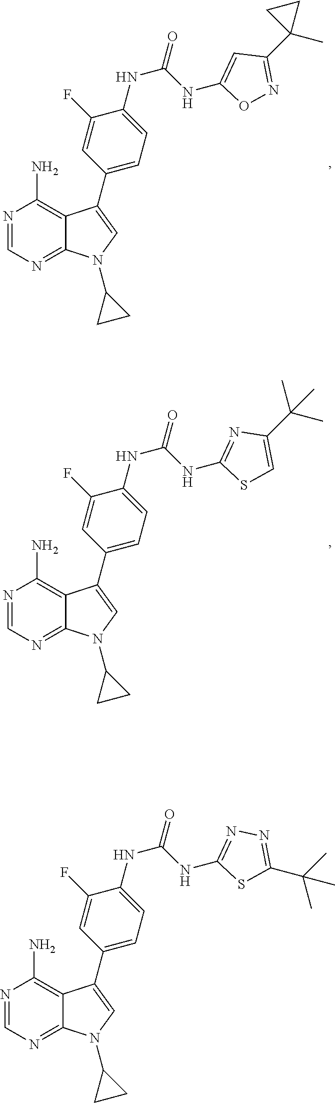 [SMILES: CC1(c2cc(NC(=O)Nc3ccc(-c4cn(C5CC5)c5ncnc(N)c45)cc3F)on2)CC1 ; CC(C)(C)c1csc(NC(=O)Nc2ccc(-c3cn(C4CC4)c4ncnc(N)c34)cc2F)n1 ; CC(C)(C)c1nnc(NC(=O)Nc2ccc(-c3cn(C4CC4)c4ncnc(N)c34)cc2F)s1]

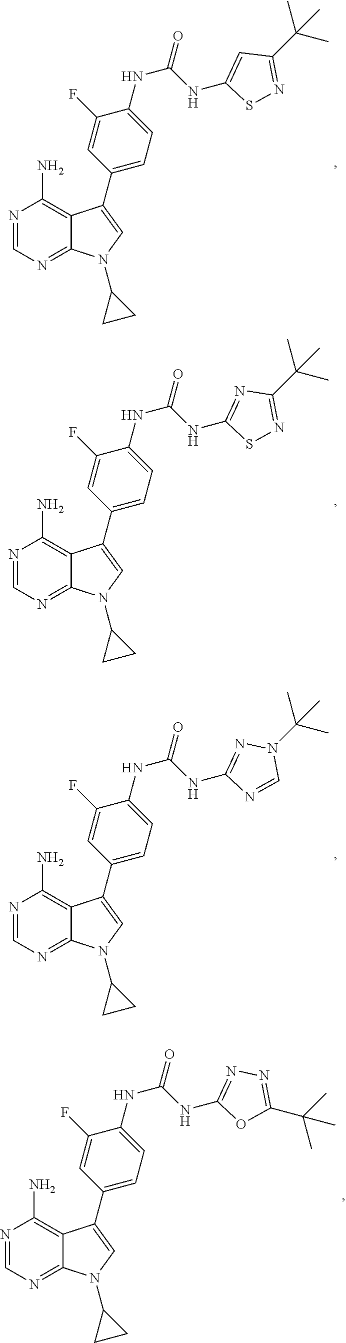 [SMILES: CC(C)(C)c1cc(NC(=O)Nc2ccc(-c3cn(C4CC4)c4ncnc(N)c34)cc2F)sn1 ; CC(C)(C)c1nsc(NC(=O)Nc2ccc(-c3cn(C4CC4)c4ncnc(N)c34)cc2F)n1 ; CC(C)(C)n1cnc(NC(=O)Nc2ccc(-c3cn(C4CC4)c4ncnc(N)c34)cc2F)n1 ; CC(C)(C)c1nnc(NC(=O)Nc2ccc(-c3cn(C4CC4)c4ncnc(N)c34)cc2F)o1]

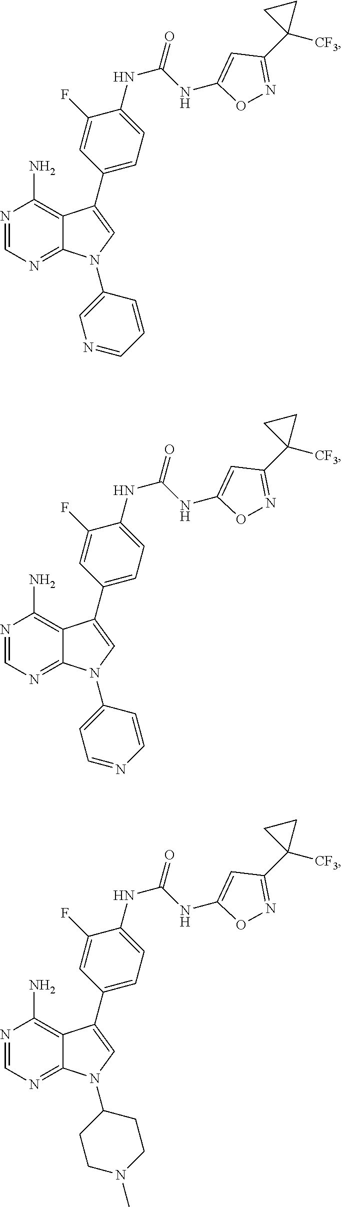 [SMILES: Nc1ncnc2c1c(-c1ccc(NC(=O)Nc3cc(C4(C(F)(F)F)CC4)no3)c(F)c1)cn2-c1cccnc1 ; Nc1ncnc2c1c(-c1ccc(NC(=O)Nc3cc(C4(C(F)(F)F)CC4)no3)c(F)c1)cn2-c1ccncc1 ; CN1CCC(n2cc(-c3ccc(NC(=O)Nc4cc(C5(C(F)(F)F)CC5)no4)c(F)c3)c3c(N)ncnc32)CC1]

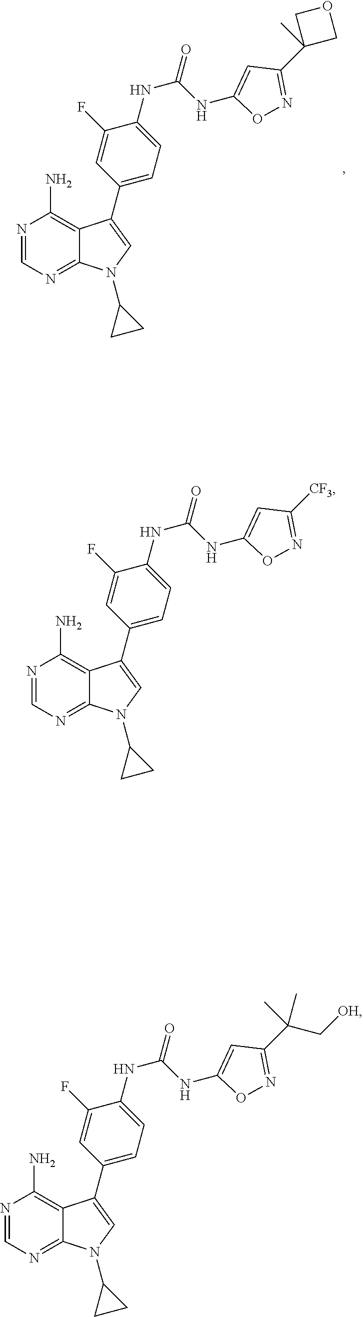 [SMILES: CC1(c2cc(NC(=O)Nc3ccc(-c4cn(C5CC5)c5ncnc(N)c45)cc3F)on2)COC1 ; Nc1ncnc2c1c(-c1ccc(NC(=O)Nc3cc(C(F)(F)F)no3)c(F)c1)cn2C1CC1 ; CC(C)(CO)c1cc(NC(=O)Nc2ccc(-c3cn(C4CC4)c4ncnc(N)c34)cc2F)on1]

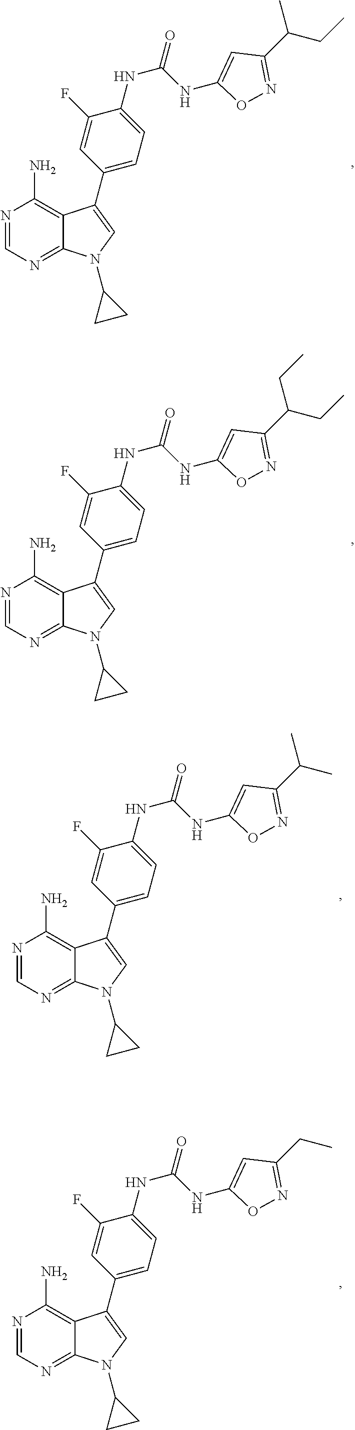 [SMILES: CCC(C)c1cc(NC(=O)Nc2ccc(-c3cn(C4CC4)c4ncnc(N)c34)cc2F)on1 ; CCC(CC)c1cc(NC(=O)Nc2ccc(-c3cn(C4CC4)c4ncnc(N)c34)cc2F)on1 ; CC(C)c1cc(NC(=O)Nc2ccc(-c3cn(C4CC4)c4ncnc(N)c34)cc2F)on1 ; CCc1cc(NC(=O)Nc2ccc(-c3cn(C4CC4)c4ncnc(N)c34)cc2F)on1]

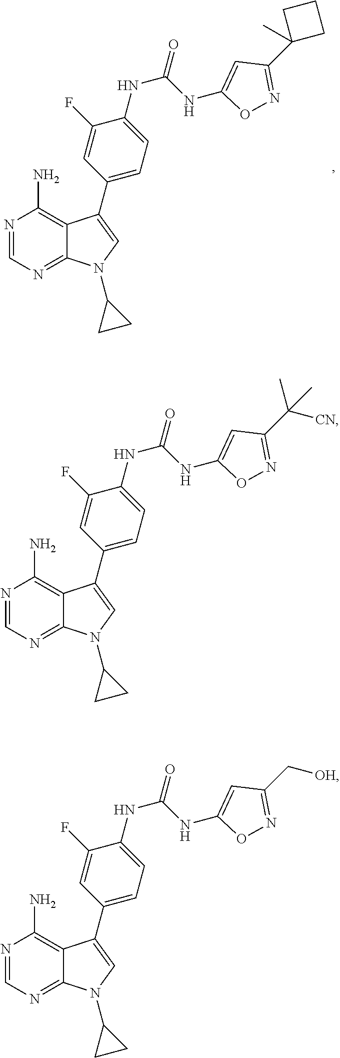 [SMILES: CC1(c2cc(NC(=O)Nc3ccc(-c4cn(C5CC5)c5ncnc(N)c45)cc3F)on2)CCC1 ; CC(C)(C#N)c1cc(NC(=O)Nc2ccc(-c3cn(C4CC4)c4ncnc(N)c34)cc2F)on1 ; Nc1ncnc2c1c(-c1ccc(NC(=O)Nc3cc(CO)no3)c(F)c1)cn2C1CC1]

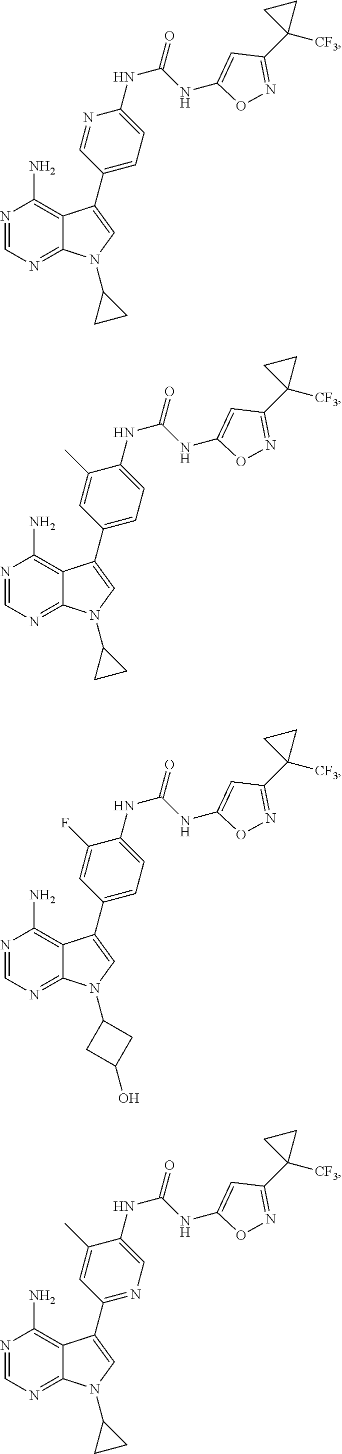 [SMILES: Nc1ncnc2c1c(-c1ccc(NC(=O)Nc3cc(C4(C(F)(F)F)CC4)no3)nc1)cn2C1CC1 ; Cc1cc(-c2cn(C3CC3)c3ncnc(N)c23)ccc1NC(=O)Nc1cc(C2(C(F)(F)F)CC2)no1 ; Nc1ncnc2c1c(-c1ccc(NC(=O)Nc3cc(C4(C(F)(F)F)CC4)no3)c(F)c1)cn2C1CC(O)C1 ; Cc1cc(-c2cn(C3CC3)c3ncnc(N)c23)ncc1NC(=O)Nc1cc(C2(C(F)(F)F)CC2)no1]

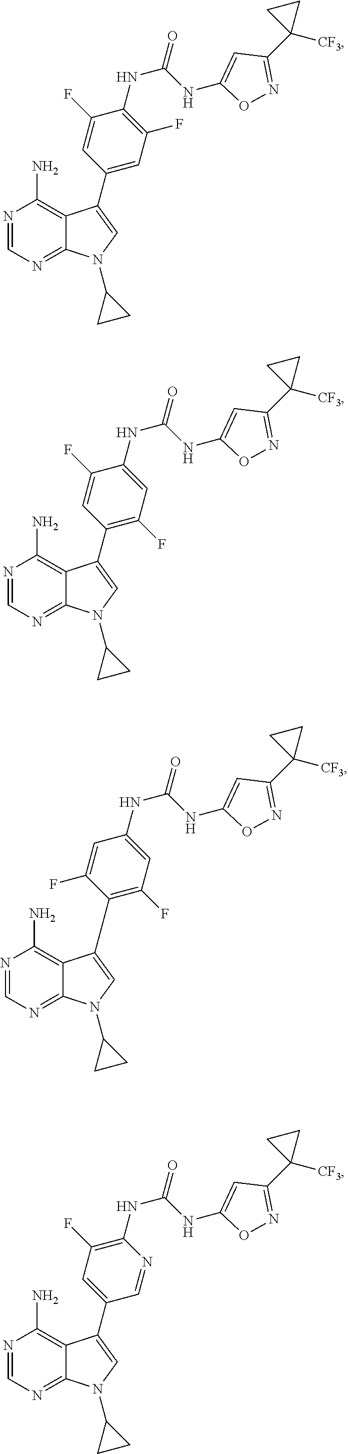 [SMILES: Nc1ncnc2c1c(-c1cc(F)c(NC(=O)Nc3cc(C4(C(F)(F)F)CC4)no3)c(F)c1)cn2C1CC1 ; Nc1ncnc2c1c(-c1cc(F)c(NC(=O)Nc3cc(C4(C(F)(F)F)CC4)no3)cc1F)cn2C1CC1 ; Nc1ncnc2c1c(-c1c(F)cc(NC(=O)Nc3cc(C4(C(F)(F)F)CC4)no3)cc1F)cn2C1CC1 ; Nc1ncnc2c1c(-c1cnc(NC(=O)Nc3cc(C4(C(F)(F)F)CC4)no3)c(F)c1)cn2C1CC1]

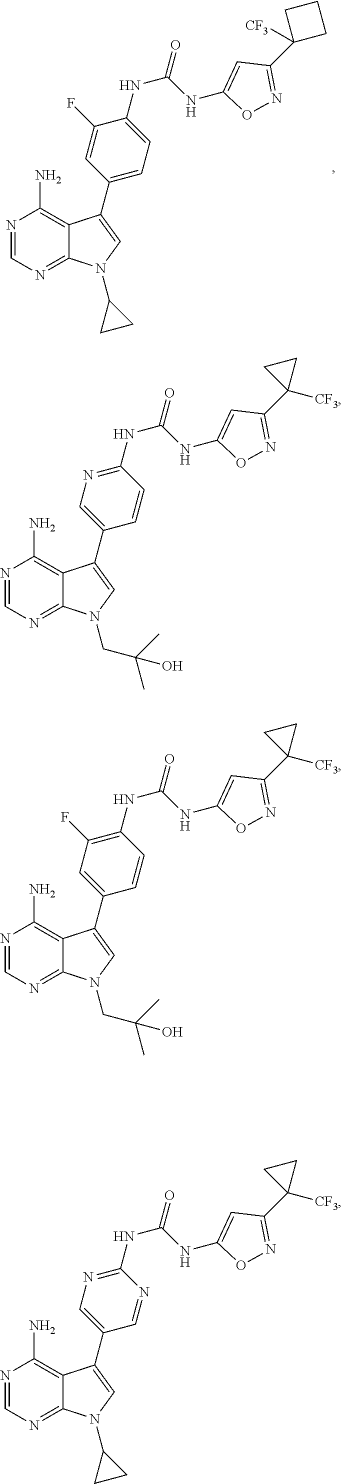 [SMILES: Nc1ncnc2c1c(-c1ccc(NC(=O)Nc3cc(C4(C(F)(F)F)CCC4)no3)c(F)c1)cn2C1CC1 ; CC(C)(O)Cn1cc(-c2ccc(NC(=O)Nc3cc(C4(C(F)(F)F)CC4)no3)nc2)c2c(N)ncnc21 ; CC(C)(O)Cn1cc(-c2ccc(NC(=O)Nc3cc(C4(C(F)(F)F)CC4)no3)c(F)c2)c2c(N)ncnc21 ; Nc1ncnc2c1c(-c1cnc(NC(=O)Nc3cc(C4(C(F)(F)F)CC4)no3)nc1)cn2C1CC1]

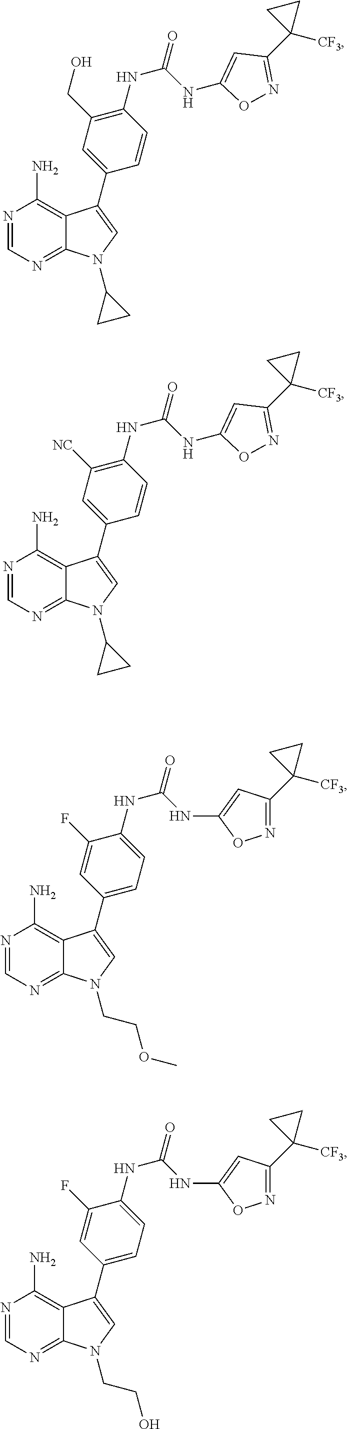 [SMILES: Nc1ncnc2c1c(-c1ccc(NC(=O)Nc3cc(C4(C(F)(F)F)CC4)no3)c(CO)c1)cn2C1CC1 ; N#Cc1cc(-c2cn(C3CC3)c3ncnc(N)c23)ccc1NC(=O)Nc1cc(C2(C(F)(F)F)CC2)no1 ; COCCn1cc(-c2ccc(NC(=O)Nc3cc(C4(C(F)(F)F)CC4)no3)c(F)c2)c2c(N)ncnc21 ; Nc1ncnc2c1c(-c1ccc(NC(=O)Nc3cc(C4(C(F)(F)F)CC4)no3)c(F)c1)cn2CCO]

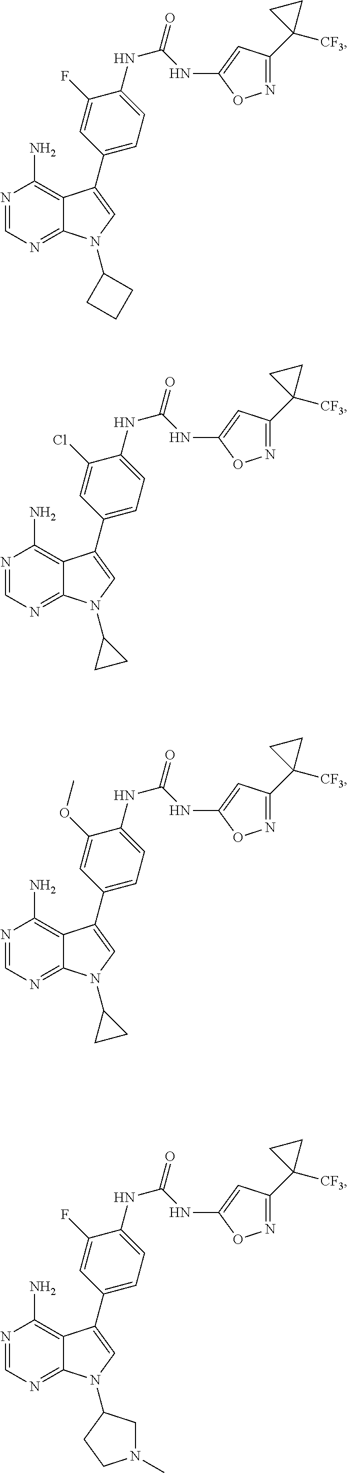 [SMILES: Nc1ncnc2c1c(-c1ccc(NC(=O)Nc3cc(C4(C(F)(F)F)CC4)no3)c(F)c1)cn2C1CCC1 ; Nc1ncnc2c1c(-c1ccc(NC(=O)Nc3cc(C4(C(F)(F)F)CC4)no3)c(Cl)c1)cn2C1CC1 ; COc1cc(-c2cn(C3CC3)c3ncnc(N)c23)ccc1NC(=O)Nc1cc(C2(C(F)(F)F)CC2)no1 ; CN1CCC(n2cc(-c3ccc(NC(=O)Nc4cc(C5(C(F)(F)F)CC5)no4)c(F)c3)c3c(N)ncnc32)C1]

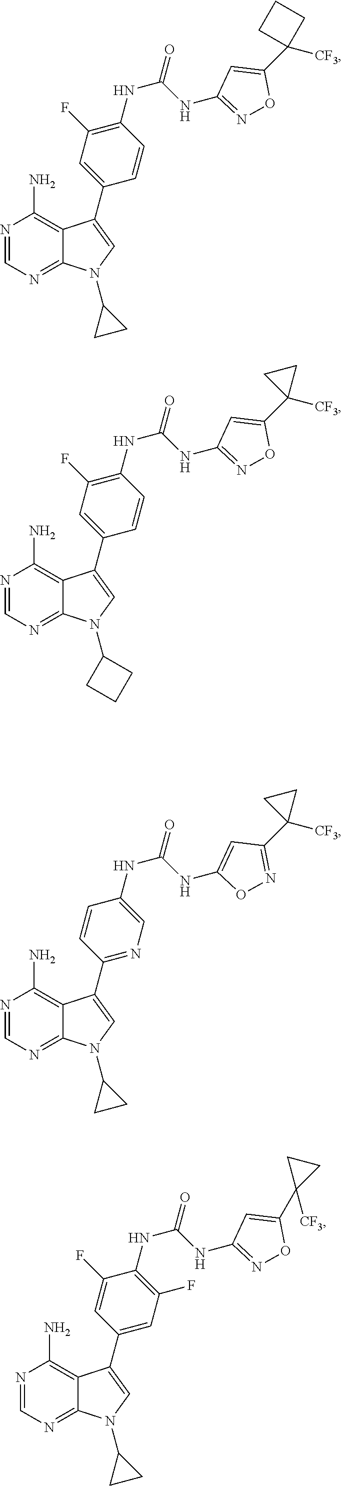 [SMILES: Nc1ncnc2c1c(-c1ccc(NC(=O)Nc3cc(C4(C(F)(F)F)CCC4)on3)c(F)c1)cn2C1CC1 ; Nc1ncnc2c1c(-c1ccc(NC(=O)Nc3cc(C4(C(F)(F)F)CC4)on3)c(F)c1)cn2C1CCC1 ; Nc1ncnc2c1c(-c1ccc(NC(=O)Nc3cc(C4(C(F)(F)F)CC4)no3)cn1)cn2C1CC1 ; Nc1ncnc2c1c(-c1cc(F)c(NC(=O)Nc3cc(C4(C(F)(F)F)CC4)on3)c(F)c1)cn2C1CC1]

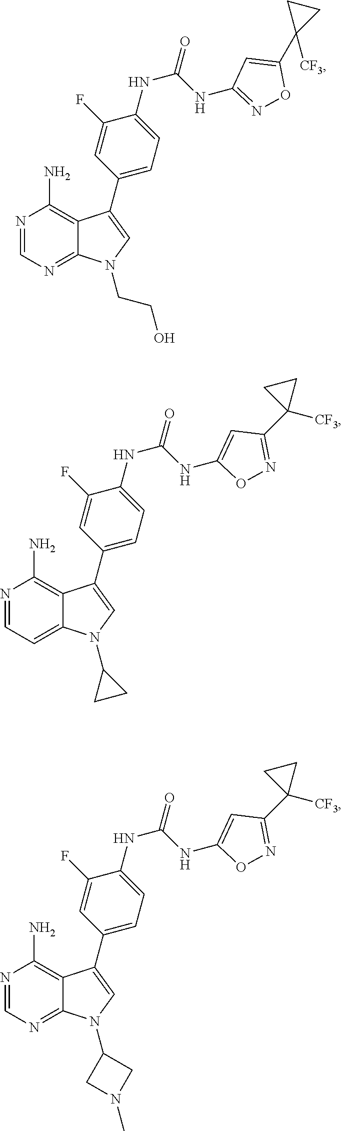 [SMILES: Nc1ncnc2c1c(-c1ccc(NC(=O)Nc3cc(C4(C(F)(F)F)CC4)on3)c(F)c1)cn2CCO ; Nc1nccc2c1c(-c1ccc(NC(=O)Nc3cc(C4(C(F)(F)F)CC4)no3)c(F)c1)cn2C1CC1 ; CN1CC(n2cc(-c3ccc(NC(=O)Nc4cc(C5(C(F)(F)F)CC5)no4)c(F)c3)c3c(N)ncnc32)C1]

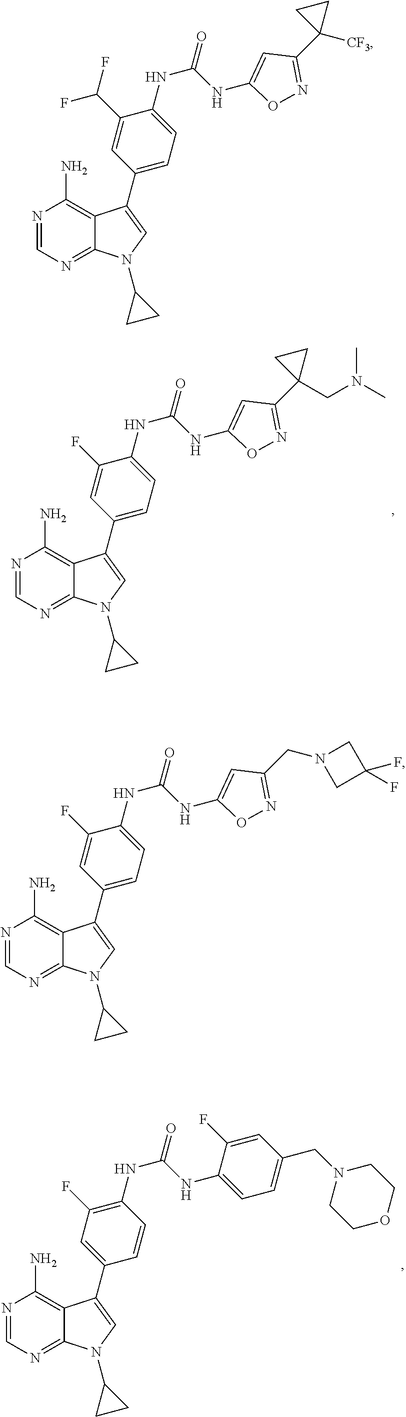 [SMILES: Nc1ncnc2c1c(-c1ccc(NC(=O)Nc3cc(C4(C(F)(F)F)CC4)no3)c(C(F)F)c1)cn2C1CC1 ; CN(C)CC1(c2cc(NC(=O)Nc3ccc(-c4cn(C5CC5)c5ncnc(N)c45)cc3F)on2)CC1 ; Nc1ncnc2c1c(-c1ccc(NC(=O)Nc3cc(CN4CC(F)(F)C4)no3)c(F)c1)cn2C1CC1 ; Nc1ncnc2c1c(-c1ccc(NC(=O)Nc3ccc(CN4CCOCC4)cc3F)c(F)c1)cn2C1CC1]

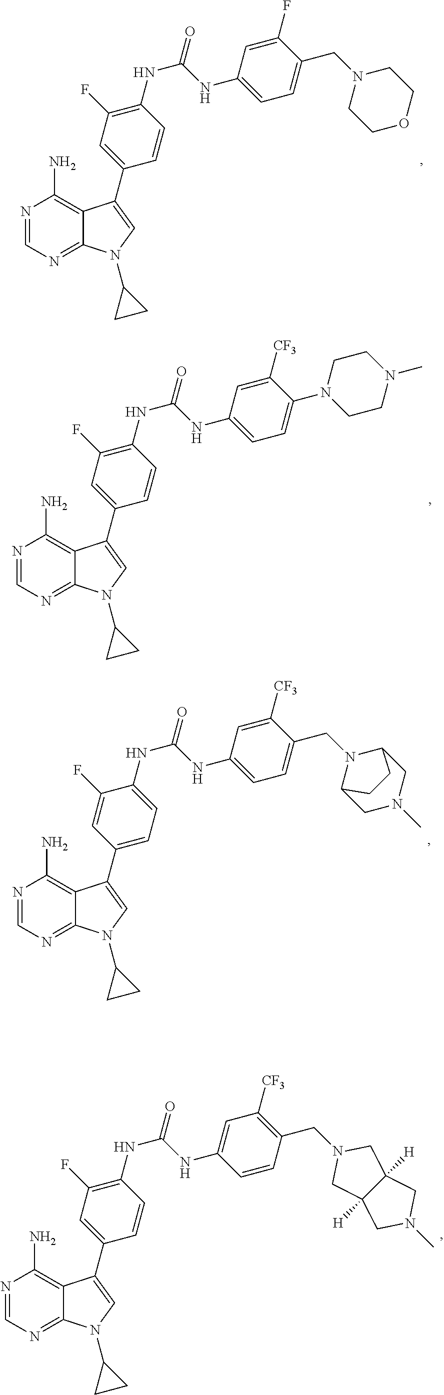 [SMILES: Nc1ncnc2c1c(-c1ccc(NC(=O)Nc3ccc(CN4CCOCC4)c(F)c3)c(F)c1)cn2C1CC1 ; CN1CCN(c2ccc(NC(=O)Nc3ccc(-c4cn(C5CC5)c5ncnc(N)c45)cc3F)cc2C(F)(F)F)CC1 ; CN1CC2CCC(C1)N2Cc1ccc(NC(=O)Nc2ccc(-c3cn(C4CC4)c4ncnc(N)c34)cc2F)cc1C(F)(F)F ; CN1C[C@@H]2CN(Cc3ccc(NC(=O)Nc4ccc(-c5cn(C6CC6)c6ncnc(N)c56)cc4F)cc3C(F)(F)F)C[C@@H]2C1]

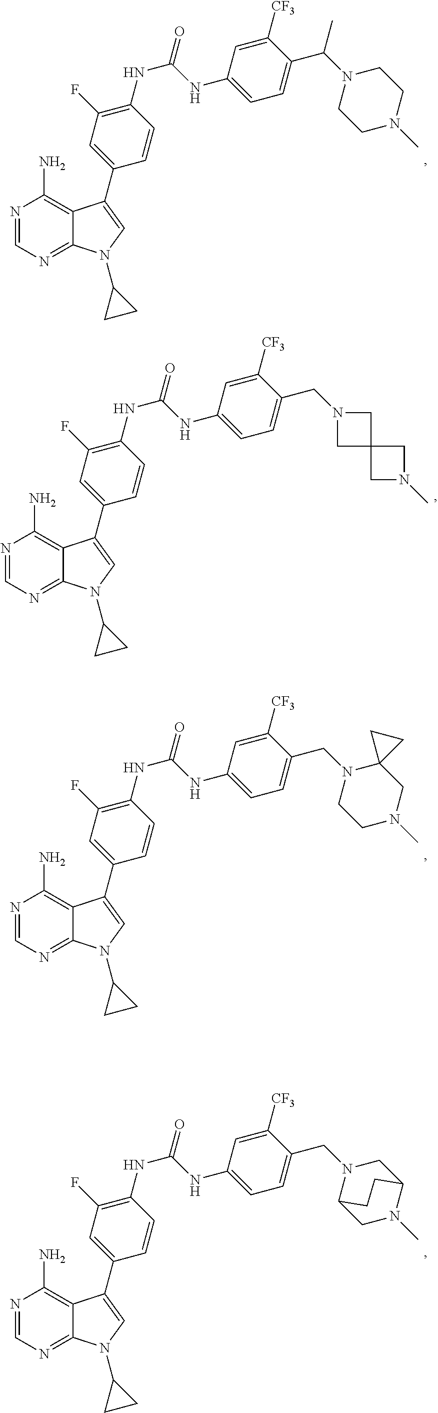 [SMILES: CC(c1ccc(NC(=O)Nc2ccc(-c3cn(C4CC4)c4ncnc(N)c34)cc2F)cc1C(F)(F)F)N1CCN(C)CC1 ; CN1CC2(C1)CN(Cc1ccc(NC(=O)Nc3ccc(-c4cn(C5CC5)c5ncnc(N)c45)cc3F)cc1C(F)(F)F)C2 ; CN1CCN(Cc2ccc(NC(=O)Nc3ccc(-c4cn(C5CC5)c5ncnc(N)c45)cc3F)cc2C(F)(F)F)C2(CC2)C1 ; CN1CC2CCC1CN2Cc1ccc(NC(=O)Nc2ccc(-c3cn(C4CC4)c4ncnc(N)c34)cc2F)cc1C(F)(F)F]

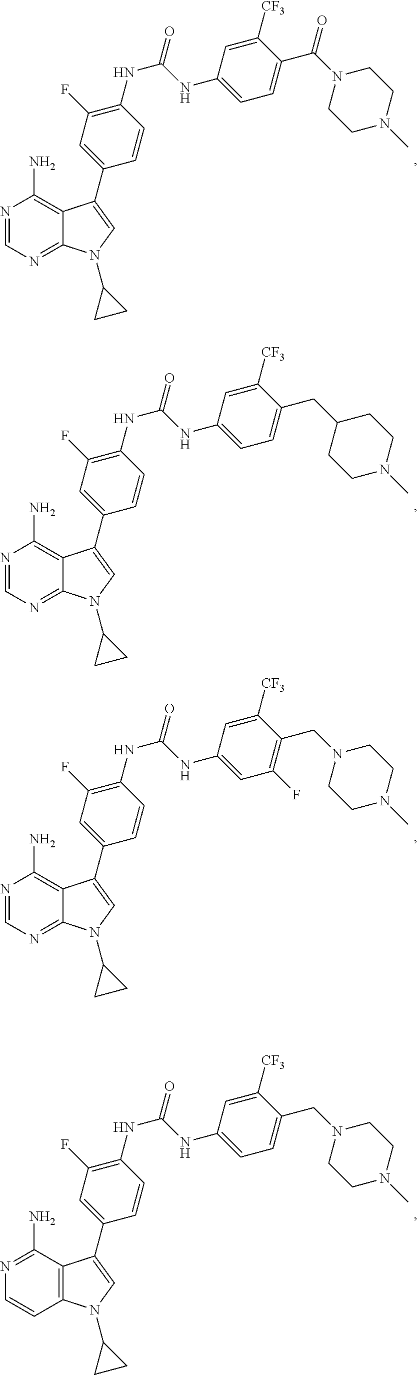 [SMILES: CN1CCN(C(=O)c2ccc(NC(=O)Nc3ccc(-c4cn(C5CC5)c5ncnc(N)c45)cc3F)cc2C(F)(F)F)CC1 ; CN1CCC(Cc2ccc(NC(=O)Nc3ccc(-c4cn(C5CC5)c5ncnc(N)c45)cc3F)cc2C(F)(F)F)CC1 ; CN1CCN(Cc2c(F)cc(NC(=O)Nc3ccc(-c4cn(C5CC5)c5ncnc(N)c45)cc3F)cc2C(F)(F)F)CC1 ; CN1CCN(Cc2ccc(NC(=O)Nc3ccc(-c4cn(C5CC5)c5ccnc(N)c45)cc3F)cc2C(F)(F)F)CC1]

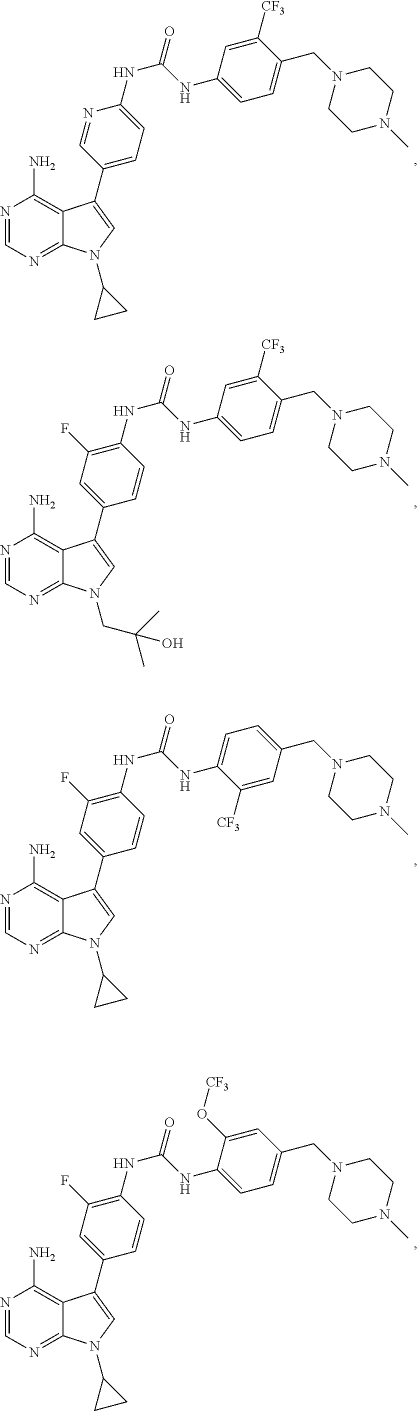 [SMILES: CN1CCN(Cc2ccc(NC(=O)Nc3ccc(-c4cn(C5CC5)c5ncnc(N)c45)cn3)cc2C(F)(F)F)CC1 ; CN1CCN(Cc2ccc(NC(=O)Nc3ccc(-c4cn(CC(C)(C)O)c5ncnc(N)c45)cc3F)cc2C(F)(F)F)CC1 ; CN1CCN(Cc2ccc(NC(=O)Nc3ccc(-c4cn(C5CC5)c5ncnc(N)c45)cc3F)c(C(F)(F)F)c2)CC1 ; CN1CCN(Cc2ccc(NC(=O)Nc3ccc(-c4cn(C5CC5)c5ncnc(N)c45)cc3F)c(OC(F)(F)F)c2)CC1]

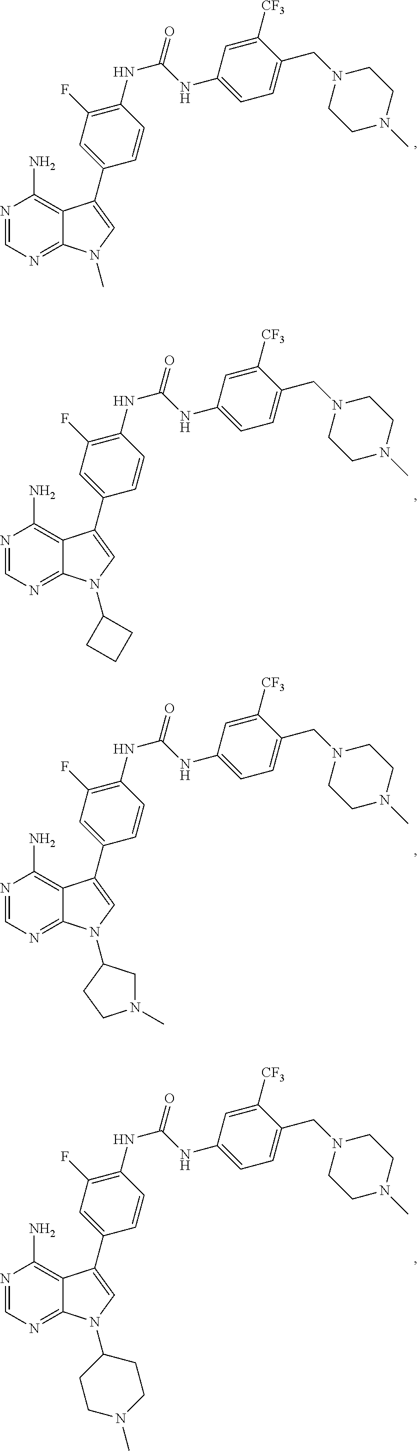 [SMILES: CN1CCN(Cc2ccc(NC(=O)Nc3ccc(-c4cn(C)c5ncnc(N)c45)cc3F)cc2C(F)(F)F)CC1 ; CN1CCN(Cc2ccc(NC(=O)Nc3ccc(-c4cn(C5CCC5)c5ncnc(N)c45)cc3F)cc2C(F)(F)F)CC1 ; CN1CCN(Cc2ccc(NC(=O)Nc3ccc(-c4cn(C5CCN(C)C5)c5ncnc(N)c45)cc3F)cc2C(F)(F)F)CC1 ; CN1CCC(n2cc(-c3ccc(NC(=O)Nc4ccc(CN5CCN(C)CC5)c(C(F)(F)F)c4)c(F)c3)c3c(N)ncnc32)CC1]

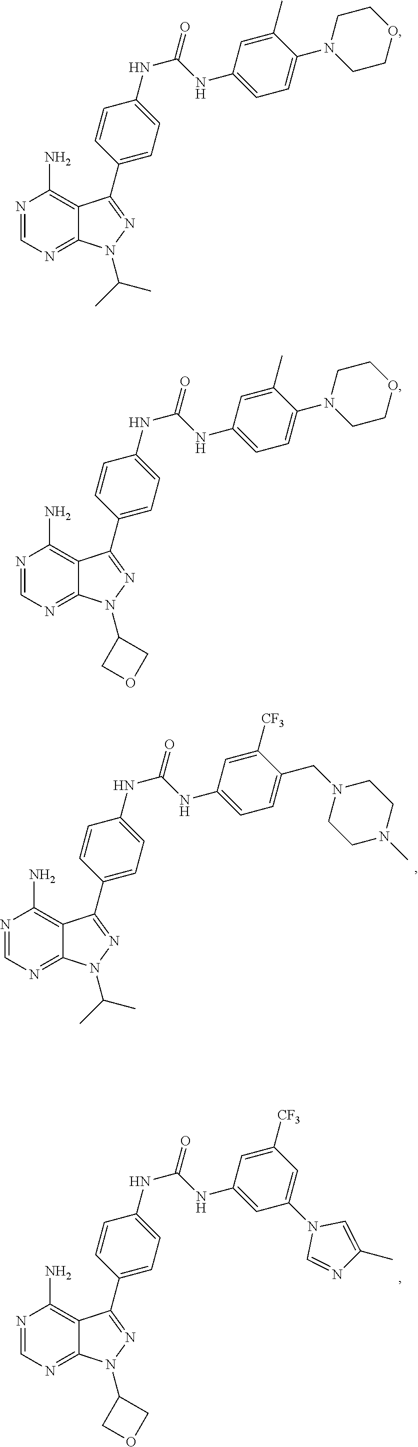 [SMILES: Cc1cc(NC(=O)Nc2ccc(-c3nn(C(C)C)c4ncnc(N)c34)cc2)ccc1N1CCOCC1 ; Cc1cc(NC(=O)Nc2ccc(-c3nn(C4COC4)c4ncnc(N)c34)cc2)ccc1N1CCOCC1 ; CC(C)n1nc(-c2ccc(NC(=O)Nc3ccc(CN4CCN(C)CC4)c(C(F)(F)F)c3)cc2)c2c(N)ncnc21 ; Cc1cn(-c2cc(NC(=O)Nc3ccc(-c4nn(C5COC5)c5ncnc(N)c45)cc3)cc(C(F)(F)F)c2)cn1]

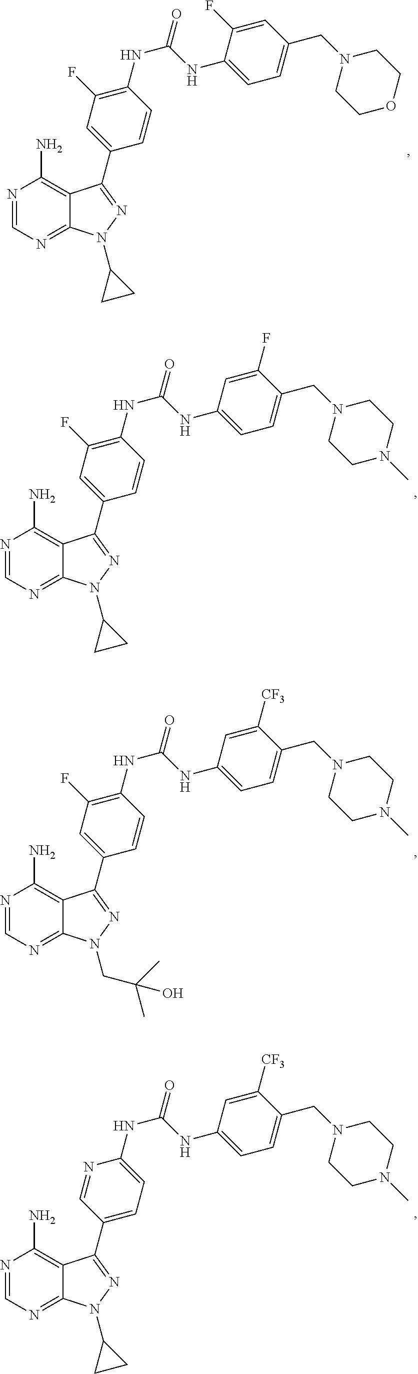 [SMILES: Nc1ncnc2c1c(-c1ccc(NC(=O)Nc3ccc(CN4CCOCC4)cc3F)c(F)c1)nn2C1CC1 ; CN1CCN(Cc2ccc(NC(=O)Nc3ccc(-c4nn(C5CC5)c5ncnc(N)c45)cc3F)cc2F)CC1 ; CN1CCN(Cc2ccc(NC(=O)Nc3ccc(-c4nn(CC(C)(C)O)c5ncnc(N)c45)cc3F)cc2C(F)(F)F)CC1 ; CN1CCN(Cc2ccc(NC(=O)Nc3ccc(-c4nn(C5CC5)c5ncnc(N)c45)cn3)cc2C(F)(F)F)CC1]

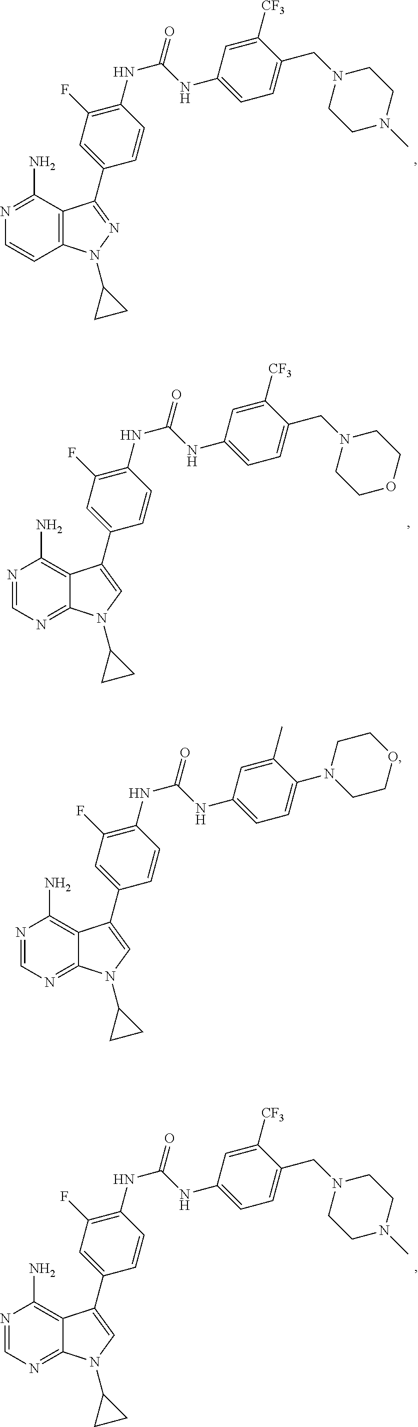 [SMILES: CN1CCN(Cc2ccc(NC(=O)Nc3ccc(-c4nn(C5CC5)c5ccnc(N)c45)cc3F)cc2C(F)(F)F)CC1 ; Nc1ncnc2c1c(-c1ccc(NC(=O)Nc3ccc(CN4CCOCC4)c(C(F)(F)F)c3)c(F)c1)cn2C1CC1 ; Cc1cc(NC(=O)Nc2ccc(-c3cn(C4CC4)c4ncnc(N)c34)cc2F)ccc1N1CCOCC1 ; CN1CCN(Cc2ccc(NC(=O)Nc3ccc(-c4cn(C5CC5)c5ncnc(N)c45)cc3F)cc2C(F)(F)F)CC1]

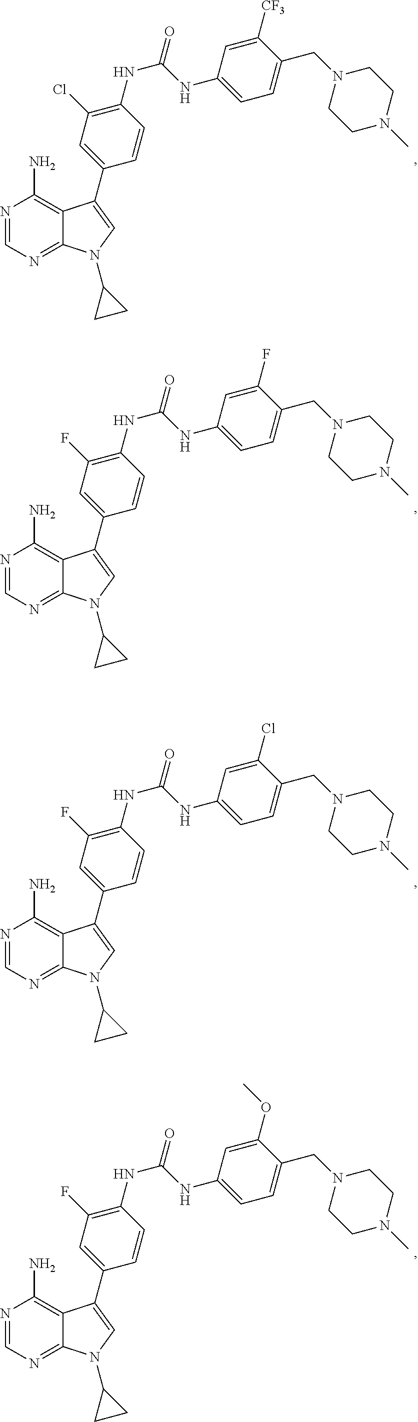 [SMILES: CN1CCN(Cc2ccc(NC(=O)Nc3ccc(-c4cn(C5CC5)c5ncnc(N)c45)cc3Cl)cc2C(F)(F)F)CC1 ; CN1CCN(Cc2ccc(NC(=O)Nc3ccc(-c4cn(C5CC5)c5ncnc(N)c45)cc3F)cc2F)CC1 ; CN1CCN(Cc2ccc(NC(=O)Nc3ccc(-c4cn(C5CC5)c5ncnc(N)c45)cc3F)cc2Cl)CC1 ; COc1cc(NC(=O)Nc2ccc(-c3cn(C4CC4)c4ncnc(N)c34)cc2F)ccc1CN1CCN(C)CC1]

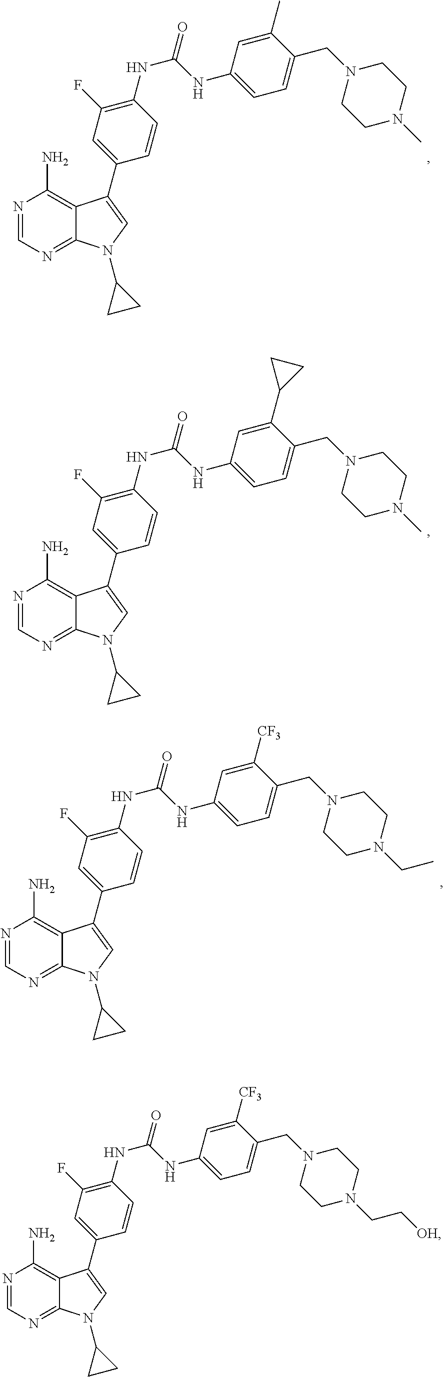 [SMILES: Cc1cc(NC(=O)Nc2ccc(-c3cn(C4CC4)c4ncnc(N)c34)cc2F)ccc1CN1CCN(C)CC1 ; CN1CCN(Cc2ccc(NC(=O)Nc3ccc(-c4cn(C5CC5)c5ncnc(N)c45)cc3F)cc2C2CC2)CC1 ; CCN1CCN(Cc2ccc(NC(=O)Nc3ccc(-c4cn(C5CC5)c5ncnc(N)c45)cc3F)cc2C(F)(F)F)CC1 ; Nc1ncnc2c1c(-c1ccc(NC(=O)Nc3ccc(CN4CCN(CCO)CC4)c(C(F)(F)F)c3)c(F)c1)cn2C1CC1]

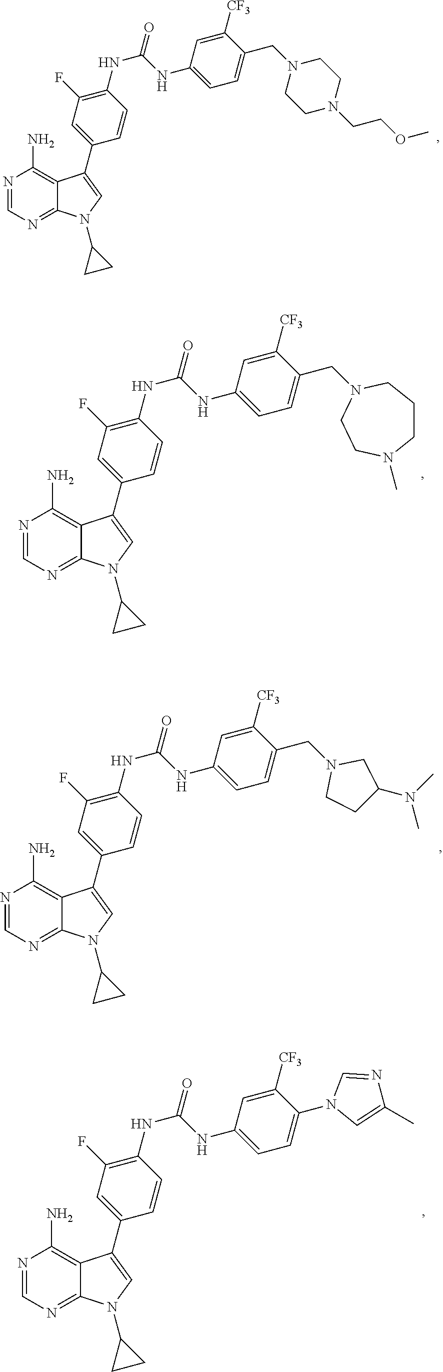 [SMILES: COCCN1CCN(Cc2ccc(NC(=O)Nc3ccc(-c4cn(C5CC5)c5ncnc(N)c45)cc3F)cc2C(F)(F)F)CC1 ; CN1CCCN(Cc2ccc(NC(=O)Nc3ccc(-c4cn(C5CC5)c5ncnc(N)c45)cc3F)cc2C(F)(F)F)CC1 ; CN(C)C1CCN(Cc2ccc(NC(=O)Nc3ccc(-c4cn(C5CC5)c5ncnc(N)c45)cc3F)cc2C(F)(F)F)C1 ; Cc1cn(-c2ccc(NC(=O)Nc3ccc(-c4cn(C5CC5)c5ncnc(N)c45)cc3F)cc2C(F)(F)F)cn1]

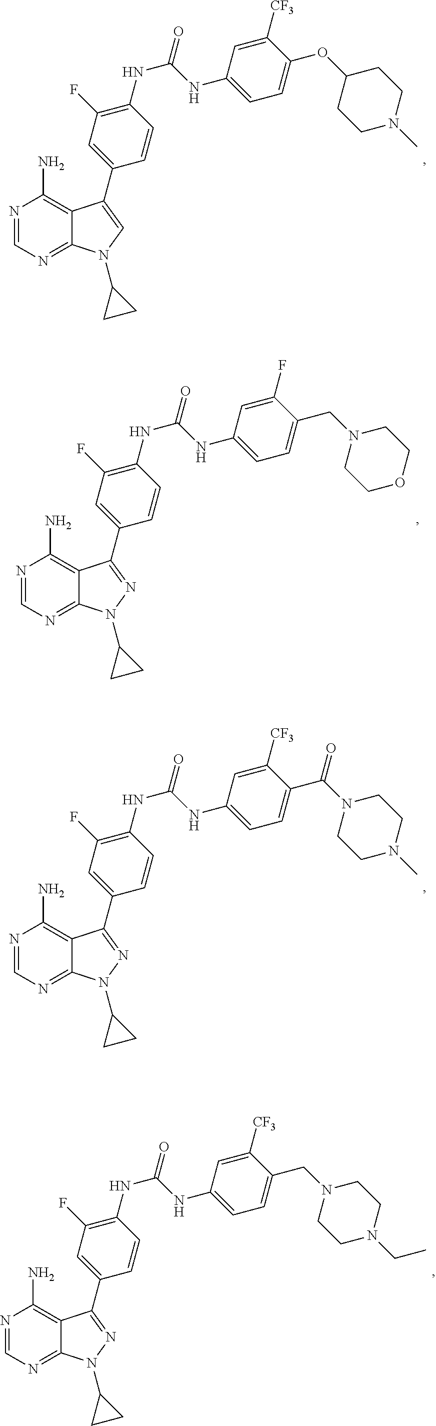 [SMILES: CN1CCC(Oc2ccc(NC(=O)Nc3ccc(-c4cn(C5CC5)c5ncnc(N)c45)cc3F)cc2C(F)(F)F)CC1 ; Nc1ncnc2c1c(-c1ccc(NC(=O)Nc3ccc(CN4CCOCC4)c(F)c3)c(F)c1)nn2C1CC1 ; CN1CCN(C(=O)c2ccc(NC(=O)Nc3ccc(-c4nn(C5CC5)c5ncnc(N)c45)cc3F)cc2C(F)(F)F)CC1 ; CCN1CCN(Cc2ccc(NC(=O)Nc3ccc(-c4nn(C5CC5)c5ncnc(N)c45)cc3F)cc2C(F)(F)F)CC1]

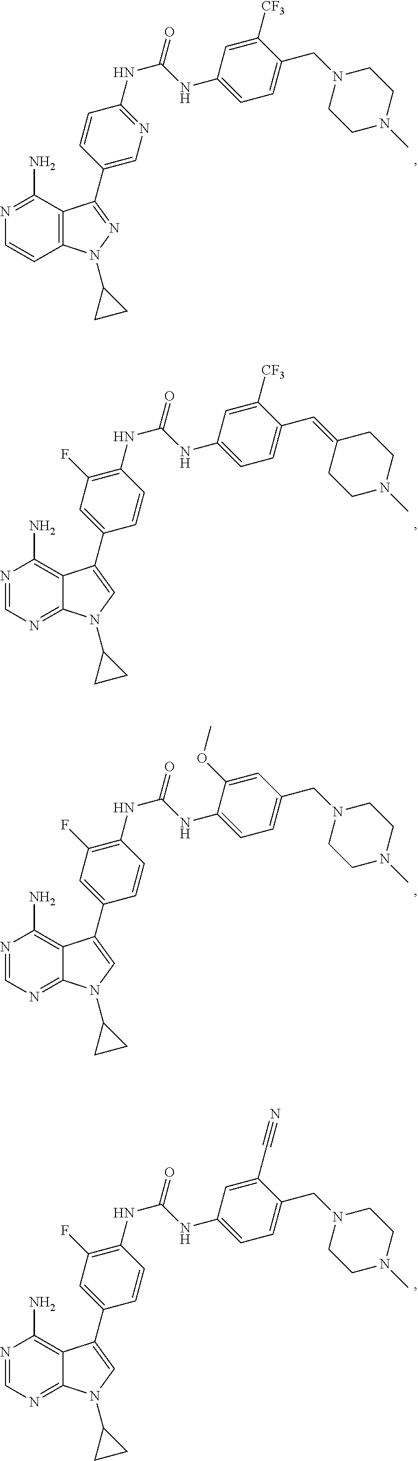 [SMILES: CN1CCN(Cc2ccc(NC(=O)Nc3ccc(-c4nn(C5CC5)c5ccnc(N)c45)cn3)cc2C(F)(F)F)CC1 ; CN1CCC(=Cc2ccc(NC(=O)Nc3ccc(-c4cn(C5CC5)c5ncnc(N)c45)cc3F)cc2C(F)(F)F)CC1 ; COc1cc(CN2CCN(C)CC2)ccc1NC(=O)Nc1ccc(-c2cn(C3CC3)c3ncnc(N)c23)cc1F ; CN1CCN(Cc2ccc(NC(=O)Nc3ccc(-c4cn(C5CC5)c5ncnc(N)c45)cc3F)cc2C#N)CC1]

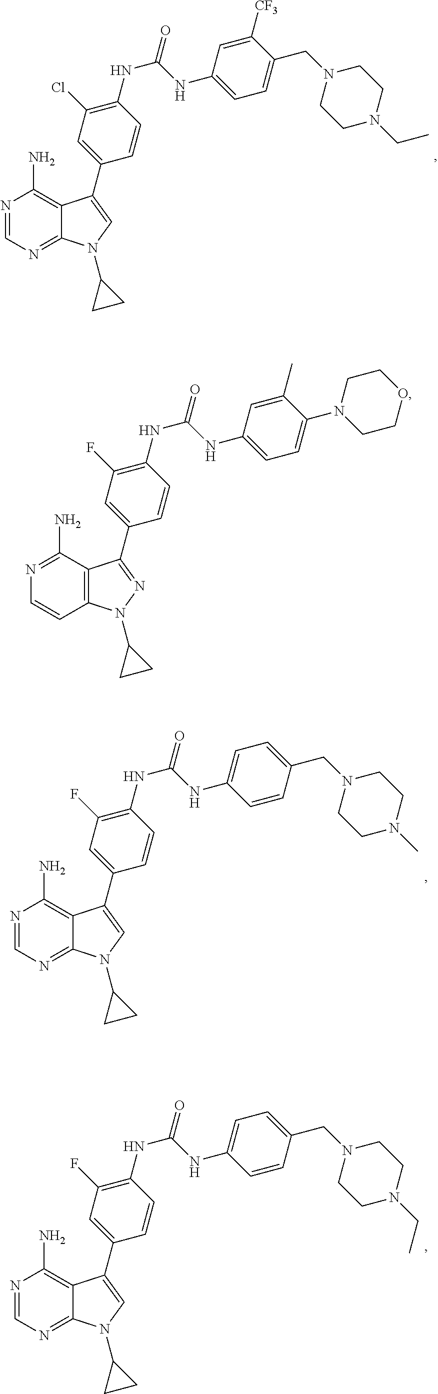 [SMILES: CCN1CCN(Cc2ccc(NC(=O)Nc3ccc(-c4cn(C5CC5)c5ncnc(N)c45)cc3Cl)cc2C(F)(F)F)CC1 ; Cc1cc(NC(=O)Nc2ccc(-c3nn(C4CC4)c4ccnc(N)c34)cc2F)ccc1N1CCOCC1 ; CN1CCN(Cc2ccc(NC(=O)Nc3ccc(-c4cn(C5CC5)c5ncnc(N)c45)cc3F)cc2)CC1 ; CCN1CCN(Cc2ccc(NC(=O)Nc3ccc(-c4cn(C5CC5)c5ncnc(N)c45)cc3F)cc2)CC1]

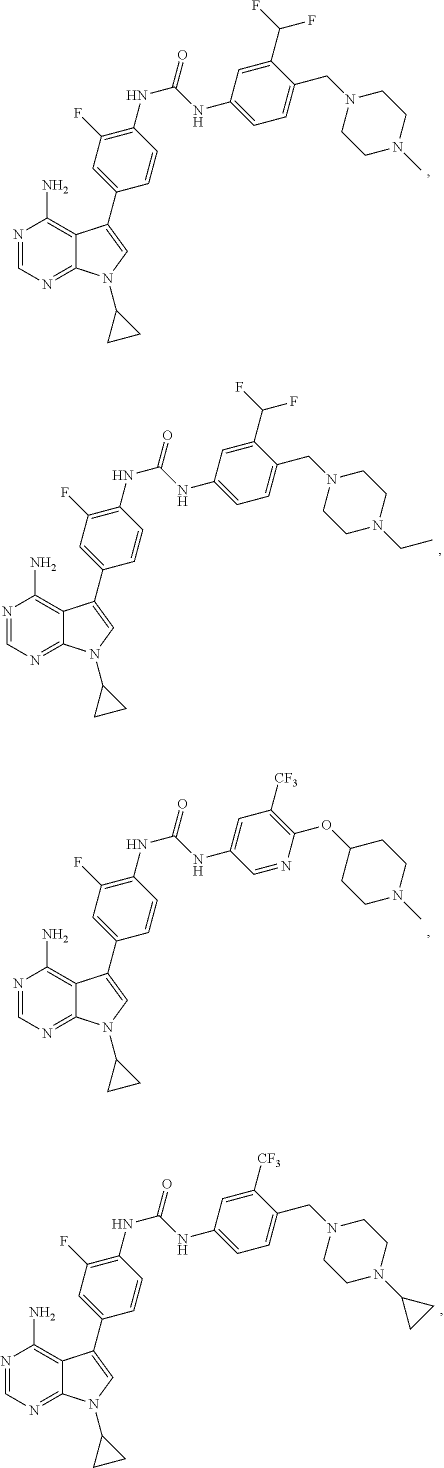 [SMILES: CN1CCN(Cc2ccc(NC(=O)Nc3ccc(-c4cn(C5CC5)c5ncnc(N)c45)cc3F)cc2C(F)F)CC1 ; CCN1CCN(Cc2ccc(NC(=O)Nc3ccc(-c4cn(C5CC5)c5ncnc(N)c45)cc3F)cc2C(F)F)CC1 ; CN1CCC(Oc2ncc(NC(=O)Nc3ccc(-c4cn(C5CC5)c5ncnc(N)c45)cc3F)cc2C(F)(F)F)CC1 ; Nc1ncnc2c1c(-c1ccc(NC(=O)Nc3ccc(CN4CCN(C5CC5)CC4)c(C(F)(F)F)c3)c(F)c1)cn2C1CC1]

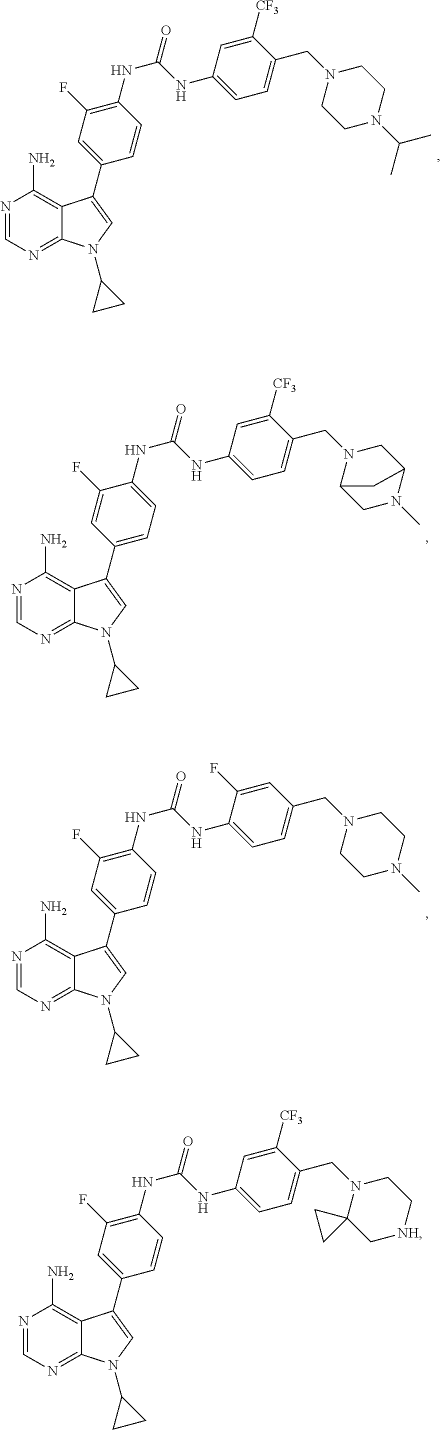 [SMILES: CC(C)N1CCN(Cc2ccc(NC(=O)Nc3ccc(-c4cn(C5CC5)c5ncnc(N)c45)cc3F)cc2C(F)(F)F)CC1 ; CN1CC2CC1CN2Cc1ccc(NC(=O)Nc2ccc(-c3cn(C4CC4)c4ncnc(N)c34)cc2F)cc1C(F)(F)F ; CN1CCN(Cc2ccc(NC(=O)Nc3ccc(-c4cn(C5CC5)c5ncnc(N)c45)cc3F)c(F)c2)CC1 ; Nc1ncnc2c1c(-c1ccc(NC(=O)Nc3ccc(CN4CCNCC45CC5)c(C(F)(F)F)c3)c(F)c1)cn2C1CC1]

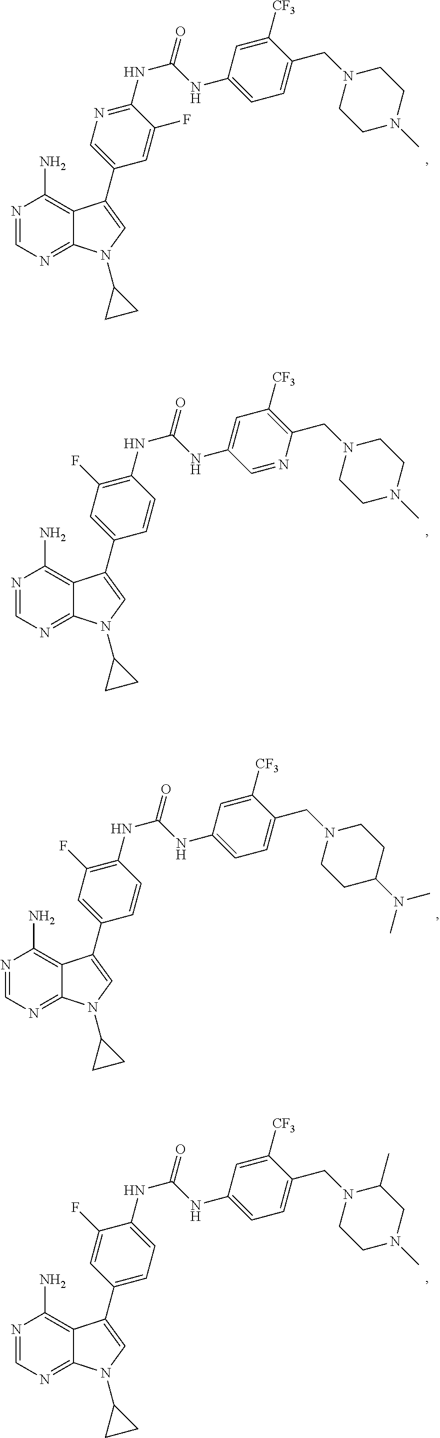 [SMILES: CN1CCN(Cc2ccc(NC(=O)Nc3ncc(-c4cn(C5CC5)c5ncnc(N)c45)cc3F)cc2C(F)(F)F)CC1 ; CN1CCN(Cc2ncc(NC(=O)Nc3ccc(-c4cn(C5CC5)c5ncnc(N)c45)cc3F)cc2C(F)(F)F)CC1 ; CN(C)C1CCN(Cc2ccc(NC(=O)Nc3ccc(-c4cn(C5CC5)c5ncnc(N)c45)cc3F)cc2C(F)(F)F)CC1 ; CC1CN(C)CCN1Cc1ccc(NC(=O)Nc2ccc(-c3cn(C4CC4)c4ncnc(N)c34)cc2F)cc1C(F)(F)F]

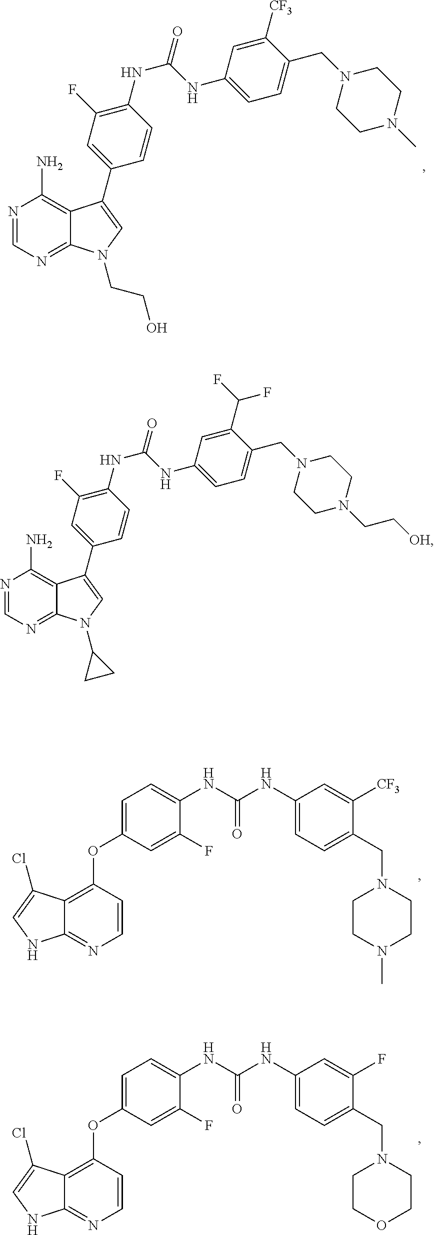 [SMILES: CN1CCN(Cc2ccc(NC(=O)Nc3ccc(-c4cn(CCO)c5ncnc(N)c45)cc3F)cc2C(F)(F)F)CC1 ; Nc1ncnc2c1c(-c1ccc(NC(=O)Nc3ccc(CN4CCN(CCO)CC4)c(C(F)F)c3)c(F)c1)cn2C1CC1 ; CN1CCN(Cc2ccc(NC(=O)Nc3ccc(Oc4ccnc5[nH]cc(Cl)c45)cc3F)cc2C(F)(F)F)CC1 ; O=C(Nc1ccc(CN2CCOCC2)c(F)c1)Nc1ccc(Oc2ccnc3[nH]cc(Cl)c23)cc1F]

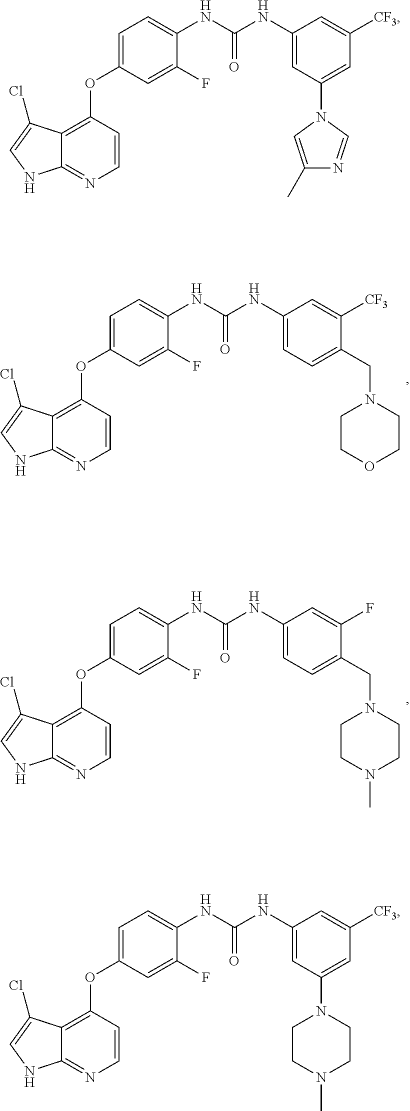 [SMILES: Cc1cn(-c2cc(NC(=O)Nc3ccc(Oc4ccnc5[nH]cc(Cl)c45)cc3F)cc(C(F)(F)F)c2)cn1 ; O=C(Nc1ccc(CN2CCOCC2)c(C(F)(F)F)c1)Nc1ccc(Oc2ccnc3[nH]cc(Cl)c23)cc1F ; CN1CCN(Cc2ccc(NC(=O)Nc3ccc(Oc4ccnc5[nH]cc(Cl)c45)cc3F)cc2F)CC1 ; CN1CCN(c2cc(NC(=O)Nc3ccc(Oc4ccnc5[nH]cc(Cl)c45)cc3F)cc(C(F)(F)F)c2)CC1]

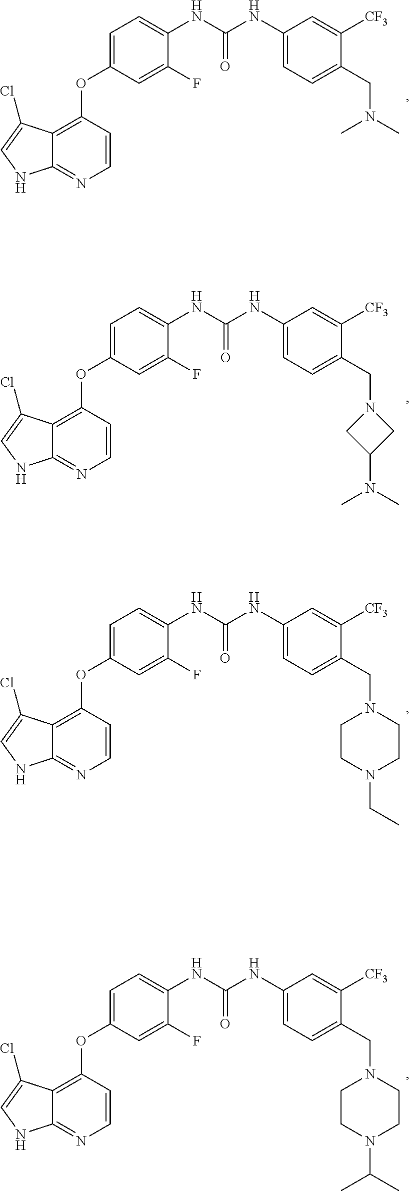 [SMILES: CN(C)Cc1ccc(NC(=O)Nc2ccc(Oc3ccnc4[nH]cc(Cl)c34)cc2F)cc1C(F)(F)F ; CN(C)C1CN(Cc2ccc(NC(=O)Nc3ccc(Oc4ccnc5[nH]cc(Cl)c45)cc3F)cc2C(F)(F)F)C1 ; CCN1CCN(Cc2ccc(NC(=O)Nc3ccc(Oc4ccnc5[nH]cc(Cl)c45)cc3F)cc2C(F)(F)F)CC1 ; CC(C)N1CCN(Cc2ccc(NC(=O)Nc3ccc(Oc4ccnc5[nH]cc(Cl)c45)cc3F)cc2C(F)(F)F)CC1]

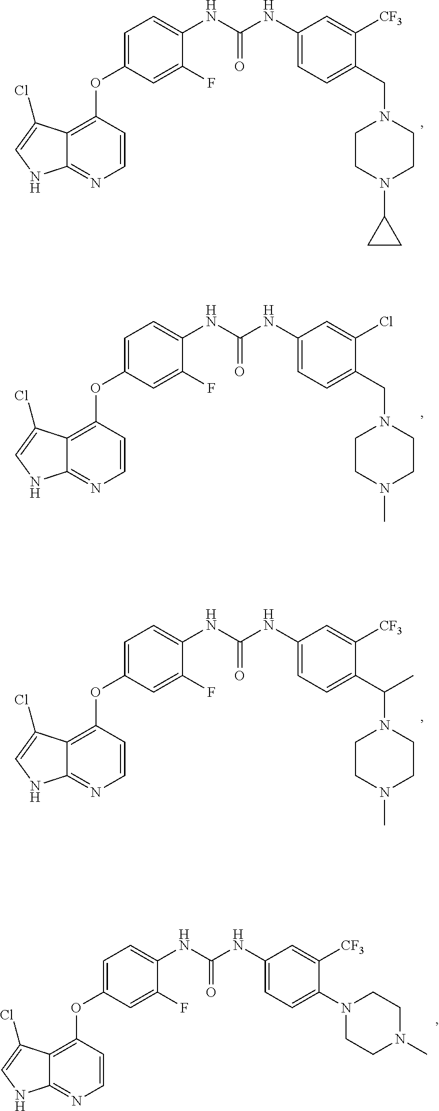 [SMILES: O=C(Nc1ccc(CN2CCN(C3CC3)CC2)c(C(F)(F)F)c1)Nc1ccc(Oc2ccnc3[nH]cc(Cl)c23)cc1F ; CN1CCN(Cc2ccc(NC(=O)Nc3ccc(Oc4ccnc5[nH]cc(Cl)c45)cc3F)cc2Cl)CC1 ; CC(c1ccc(NC(=O)Nc2ccc(Oc3ccnc4[nH]cc(Cl)c34)cc2F)cc1C(F)(F)F)N1CCN(C)CC1 ; CN1CCN(c2ccc(NC(=O)Nc3ccc(Oc4ccnc5[nH]cc(Cl)c45)cc3F)cc2C(F)(F)F)CC1]

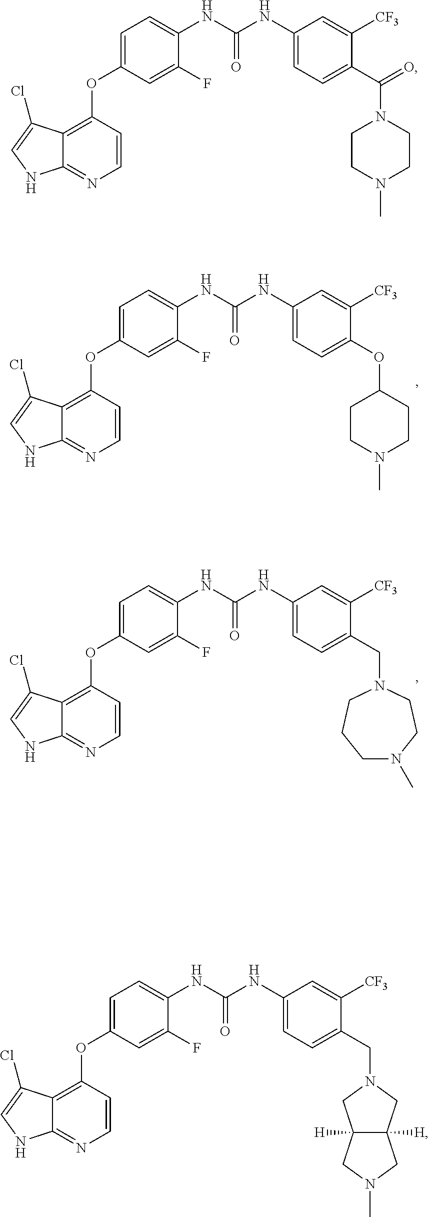

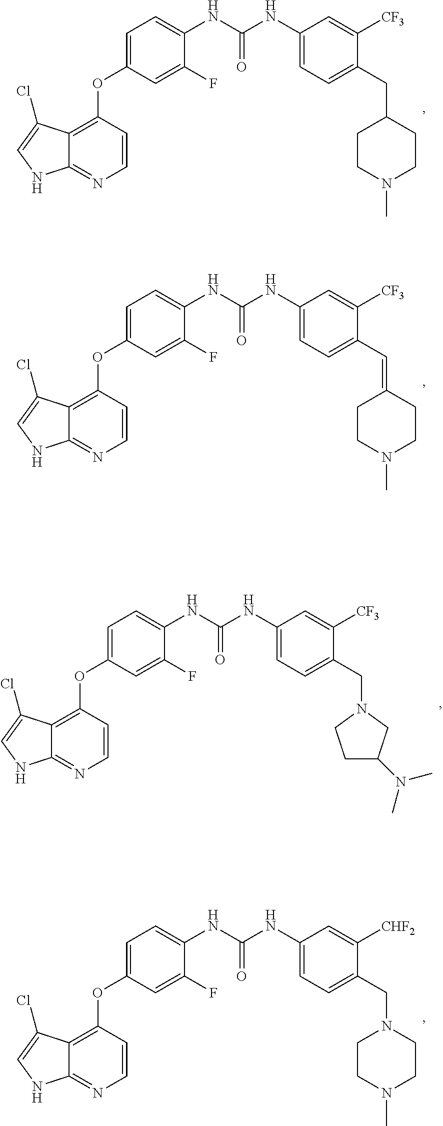 [SMILES: CN1CCC(Cc2ccc(NC(=O)Nc3ccc(Oc4ccnc5[nH]cc(Cl)c45)cc3F)cc2C(F)(F)F)CC1 ; CN1CCC(=Cc2ccc(NC(=O)Nc3ccc(Oc4ccnc5[nH]cc(Cl)c45)cc3F)cc2C(F)(F)F)CC1 ; CN(C)C1CCN(Cc2ccc(NC(=O)Nc3ccc(Oc4ccnc5[nH]cc(Cl)c45)cc3F)cc2C(F)(F)F)C1 ; CN1CCN(Cc2ccc(NC(=O)Nc3ccc(Oc4ccnc5[nH]cc(Cl)c45)cc3F)cc2C(F)F)CC1]

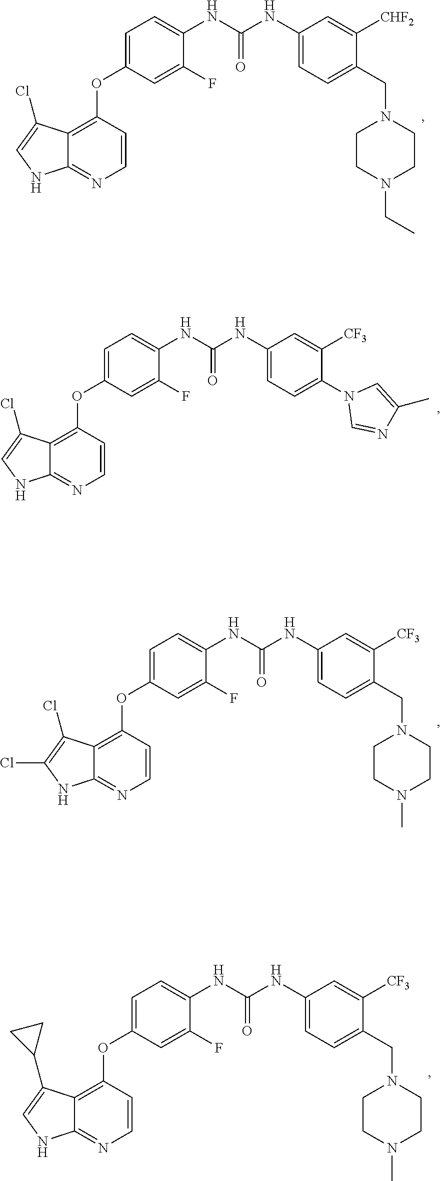 [SMILES: CCN1CCN(Cc2ccc(NC(=O)Nc3ccc(Oc4ccnc5[nH]cc(Cl)c45)cc3F)cc2C(F)F)CC1 ; Cc1cn(-c2ccc(NC(=O)Nc3ccc(Oc4ccnc5[nH]cc(Cl)c45)cc3F)cc2C(F)(F)F)cn1 ; CN1CCN(Cc2ccc(NC(=O)Nc3ccc(Oc4ccnc5[nH]c(Cl)c(Cl)c45)cc3F)cc2C(F)(F)F)CC1 ; CN1CCN(Cc2ccc(NC(=O)Nc3ccc(Oc4ccnc5[nH]cc(C6CC6)c45)cc3F)cc2C(F)(F)F)CC1]

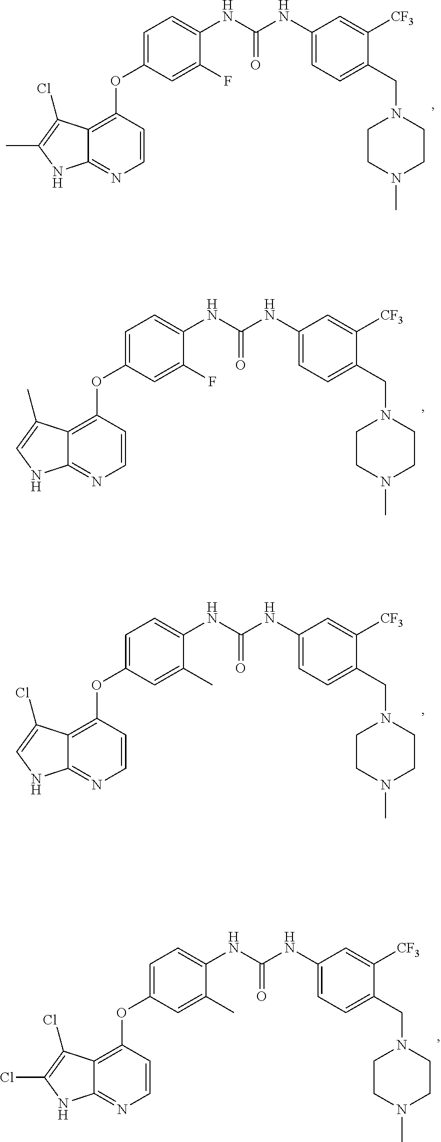 [SMILES: Cc1[nH]c2nccc(Oc3ccc(NC(=O)Nc4ccc(CN5CCN(C)CC5)c(C(F)(F)F)c4)c(F)c3)c2c1Cl ; Cc1c[nH]c2nccc(Oc3ccc(NC(=O)Nc4ccc(CN5CCN(C)CC5)c(C(F)(F)F)c4)c(F)c3)c12 ; Cc1cc(Oc2ccnc3[nH]cc(Cl)c23)ccc1NC(=O)Nc1ccc(CN2CCN(C)CC2)c(C(F)(F)F)c1 ; Cc1cc(Oc2ccnc3[nH]c(Cl)c(Cl)c23)ccc1NC(=O)Nc1ccc(CN2CCN(C)CC2)c(C(F)(F)F)c1]

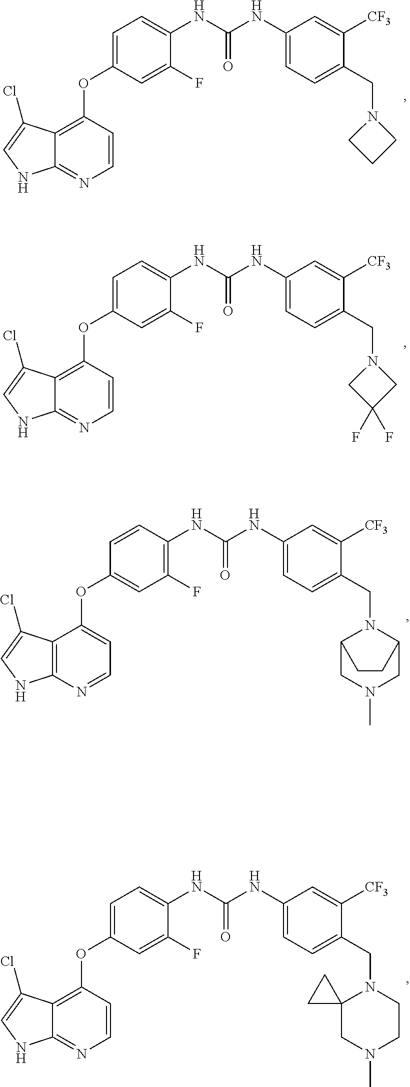 [SMILES: O=C(Nc1ccc(CN2CCC2)c(C(F)(F)F)c1)Nc1ccc(Oc2ccnc3[nH]cc(Cl)c23)cc1F ; O=C(Nc1ccc(CN2CC(F)(F)C2)c(C(F)(F)F)c1)Nc1ccc(Oc2ccnc3[nH]cc(Cl)c23)cc1F ; CN1CC2CCC(C1)N2Cc1ccc(NC(=O)Nc2ccc(Oc3ccnc4[nH]cc(Cl)c34)cc2F)cc1C(F)(F)F ; CN1CCN(Cc2ccc(NC(=O)Nc3ccc(Oc4ccnc5[nH]cc(Cl)c45)cc3F)cc2C(F)(F)F)C2(CC2)C1]

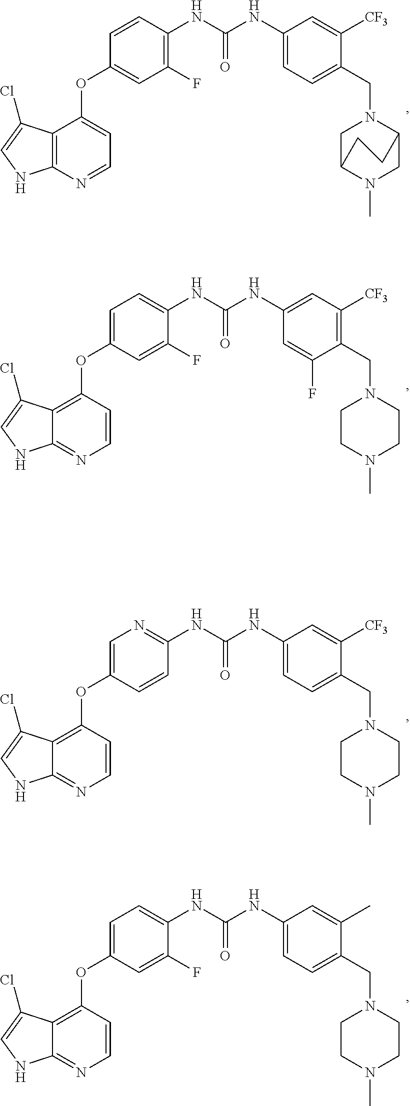 [SMILES: CN1CC2CCC1CN2Cc1ccc(NC(=O)Nc2ccc(Oc3ccnc4[nH]cc(Cl)c34)cc2F)cc1C(F)(F)F ; CN1CCN(Cc2c(F)cc(NC(=O)Nc3ccc(Oc4ccnc5[nH]cc(Cl)c45)cc3F)cc2C(F)(F)F)CC1 ; CN1CCN(Cc2ccc(NC(=O)Nc3ccc(Oc4ccnc5[nH]cc(Cl)c45)cn3)cc2C(F)(F)F)CC1 ; Cc1cc(NC(=O)Nc2ccc(Oc3ccnc4[nH]cc(Cl)c34)cc2F)ccc1CN1CCN(C)CC1]

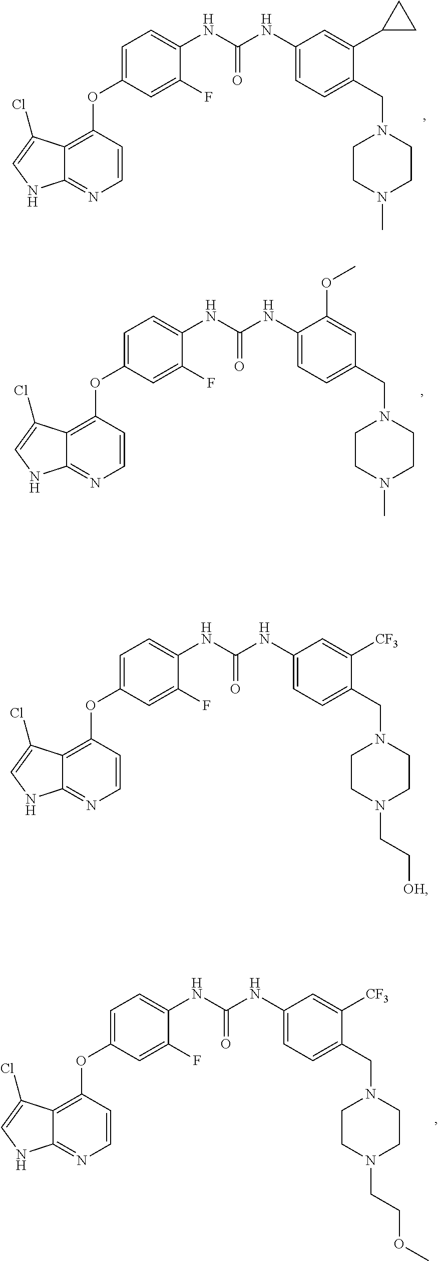

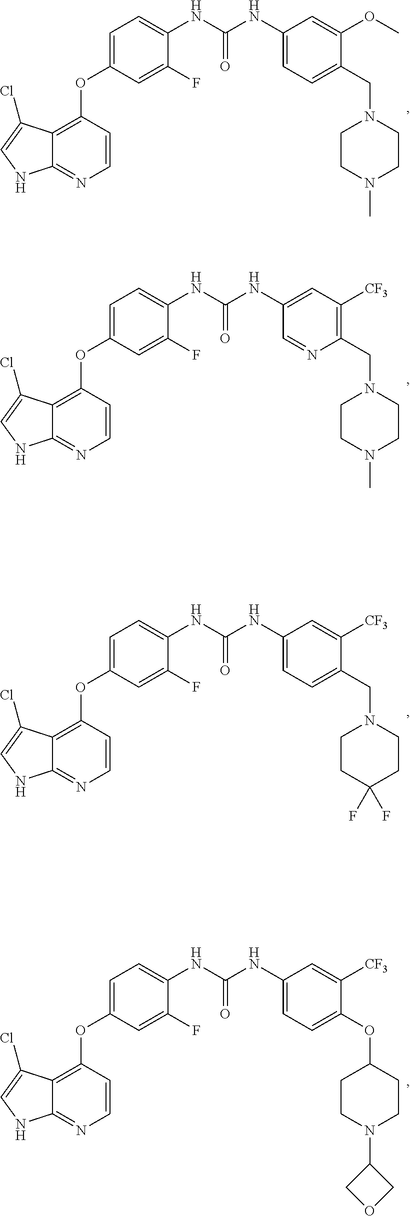 [SMILES: COc1cc(NC(=O)Nc2ccc(Oc3ccnc4[nH]cc(Cl)c34)cc2F)ccc1CN1CCN(C)CC1 ; CN1CCN(Cc2ncc(NC(=O)Nc3ccc(Oc4ccnc5[nH]cc(Cl)c45)cc3F)cc2C(F)(F)F)CC1 ; O=C(Nc1ccc(CN2CCC(F)(F)CC2)c(C(F)(F)F)c1)Nc1ccc(Oc2ccnc3[nH]cc(Cl)c23)cc1F ; O=C(Nc1ccc(OC2CCN(C3COC3)CC2)c(C(F)(F)F)c1)Nc1ccc(Oc2ccnc3[nH]cc(Cl)c23)cc1F]

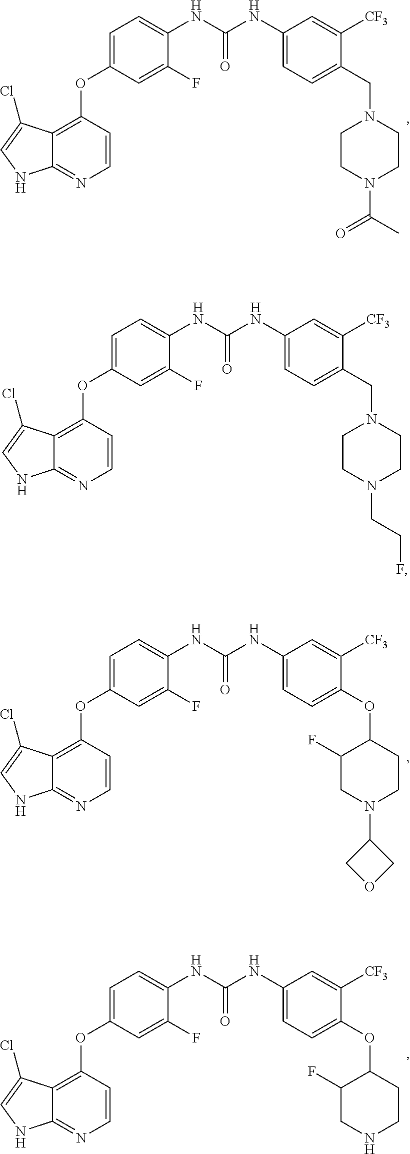 [SMILES: CC(=O)N1CCN(Cc2ccc(NC(=O)Nc3ccc(Oc4ccnc5[nH]cc(Cl)c45)cc3F)cc2C(F)(F)F)CC1 ; O=C(Nc1ccc(CN2CCN(CCF)CC2)c(C(F)(F)F)c1)Nc1ccc(Oc2ccnc3[nH]cc(Cl)c23)cc1F ; O=C(Nc1ccc(OC2CCN(C3COC3)CC2F)c(C(F)(F)F)c1)Nc1ccc(Oc2ccnc3[nH]cc(Cl)c23)cc1F ; O=C(Nc1ccc(OC2CCNCC2F)c(C(F)(F)F)c1)Nc1ccc(Oc2ccnc3[nH]cc(Cl)c23)cc1F]

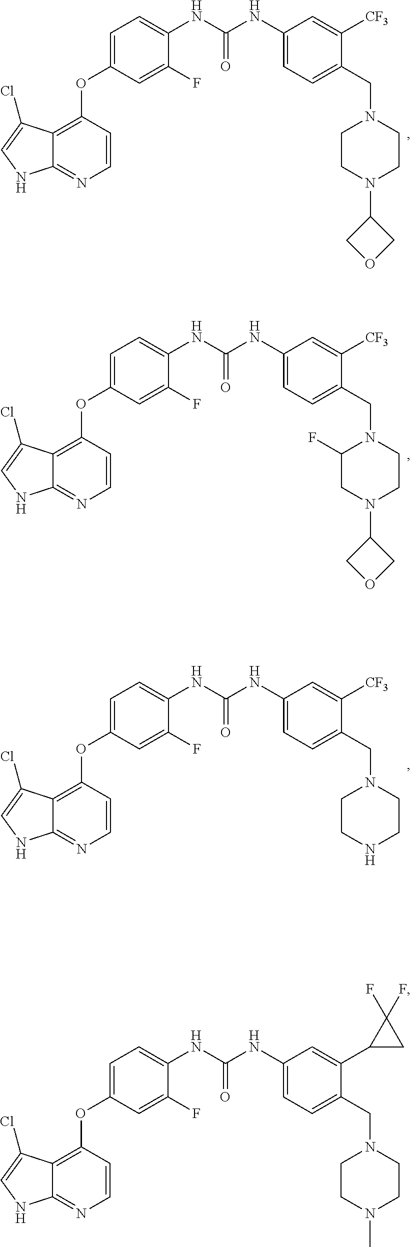 [SMILES: O=C(Nc1ccc(CN2CCN(C3COC3)CC2)c(C(F)(F)F)c1)Nc1ccc(Oc2ccnc3[nH]cc(Cl)c23)cc1F ; O=C(Nc1ccc(CN2CCN(C3COC3)CC2F)c(C(F)(F)F)c1)Nc1ccc(Oc2ccnc3[nH]cc(Cl)c23)cc1F ; O=C(Nc1ccc(CN2CCNCC2)c(C(F)(F)F)c1)Nc1ccc(Oc2ccnc3[nH]cc(Cl)c23)cc1F ; CN1CCN(Cc2ccc(NC(=O)Nc3ccc(Oc4ccnc5[nH]cc(Cl)c45)cc3F)cc2C2CC2(F)F)CC1]

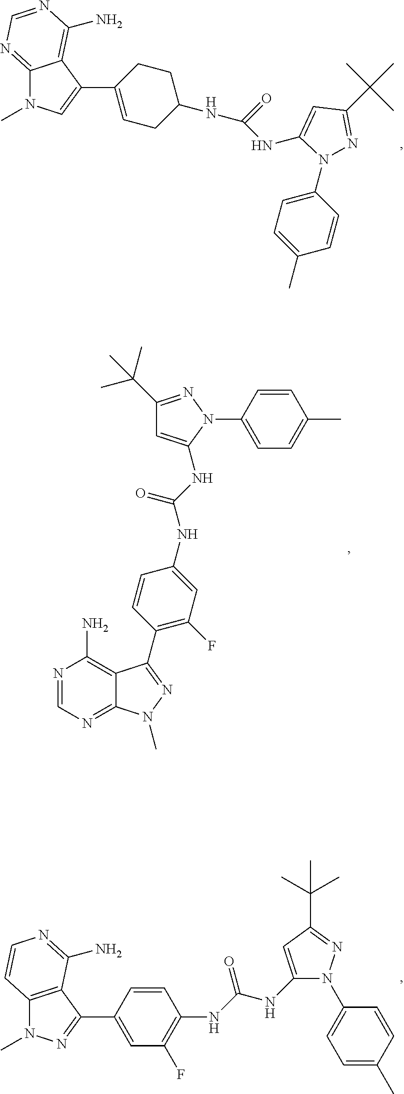

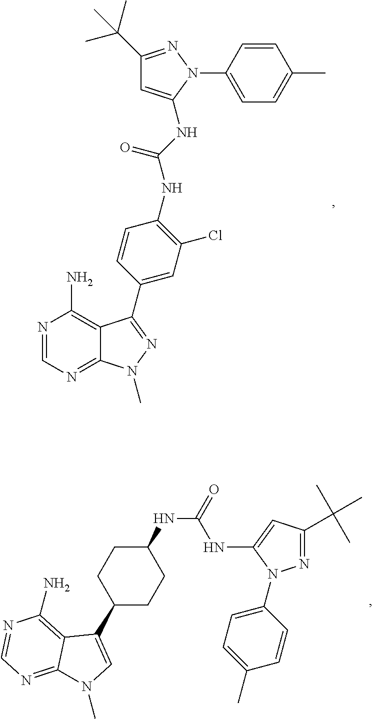 [SMILES: Cc1ccc(-n2nc(C(C)(C)C)cc2NC(=O)Nc2ccc(-c3nn(C)c4ncnc(N)c34)cc2Cl)cc1 ; Cc1ccc(-n2nc(C(C)(C)C)cc2NC(=O)N[C@H]2CC[C@@H](c3cn(C)c4ncnc(N)c43)CC2)cc1]

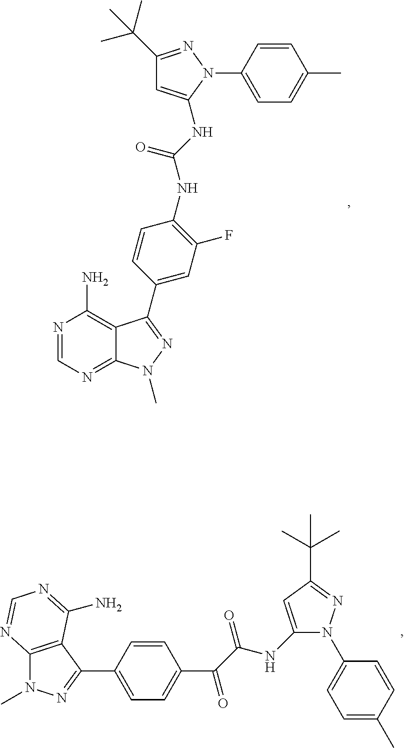

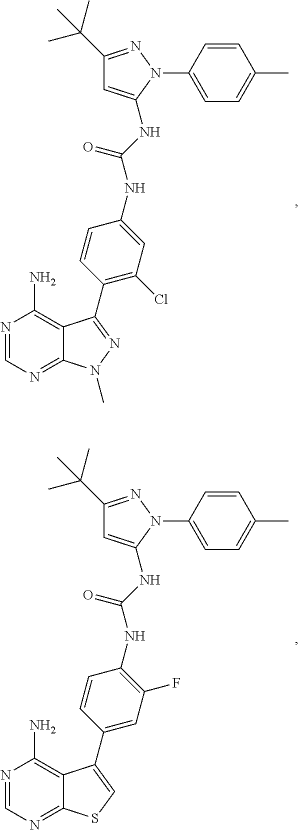 [SMILES: Cc1ccc(-n2nc(C(C)(C)C)cc2NC(=O)Nc2ccc(-c3nn(C)c4ncnc(N)c34)c(Cl)c2)cc1 ; Cc1ccc(-n2nc(C(C)(C)C)cc2NC(=O)Nc2ccc(-c3csc4ncnc(N)c34)cc2F)cc1]

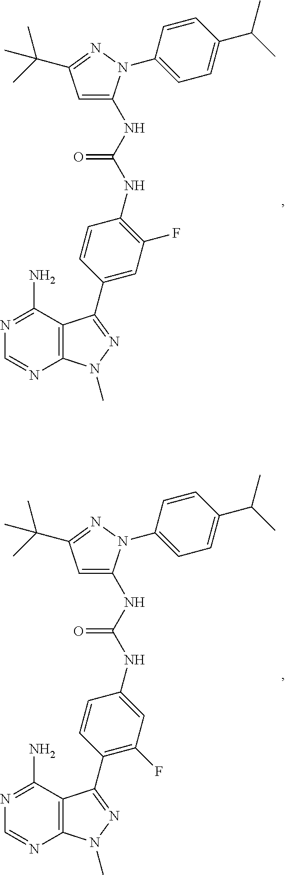

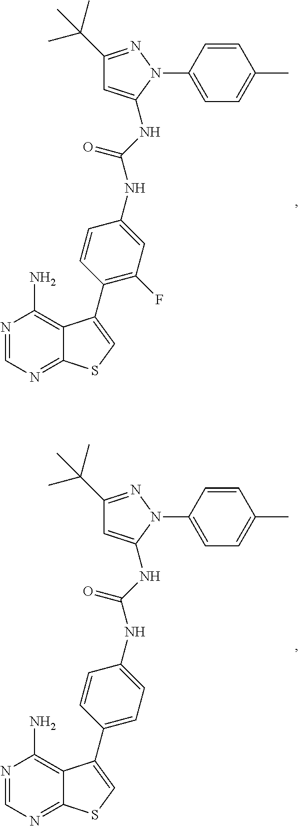 [SMILES: Cc1ccc(-n2nc(C(C)(C)C)cc2NC(=O)Nc2ccc(-c3csc4ncnc(N)c34)c(F)c2)cc1 ; Cc1ccc(-n2nc(C(C)(C)C)cc2NC(=O)Nc2ccc(-c3csc4ncnc(N)c34)cc2)cc1]

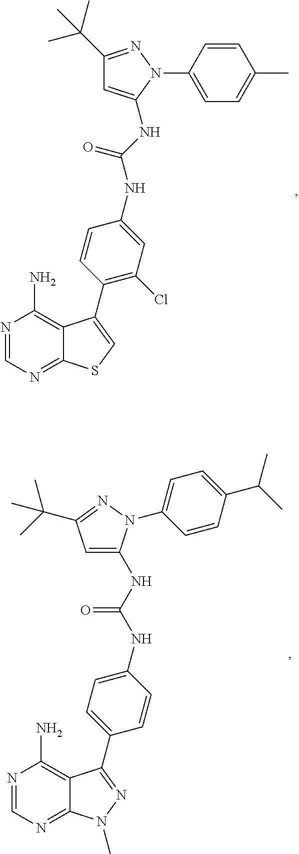

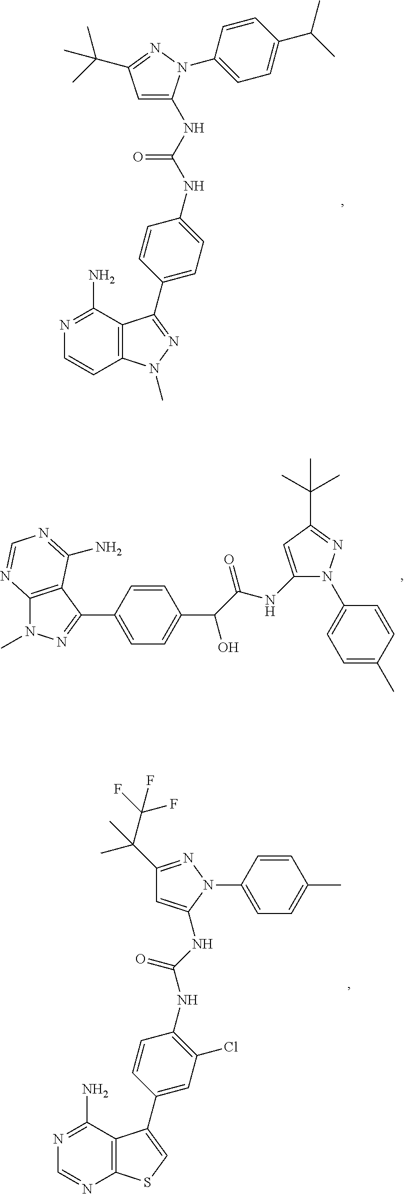

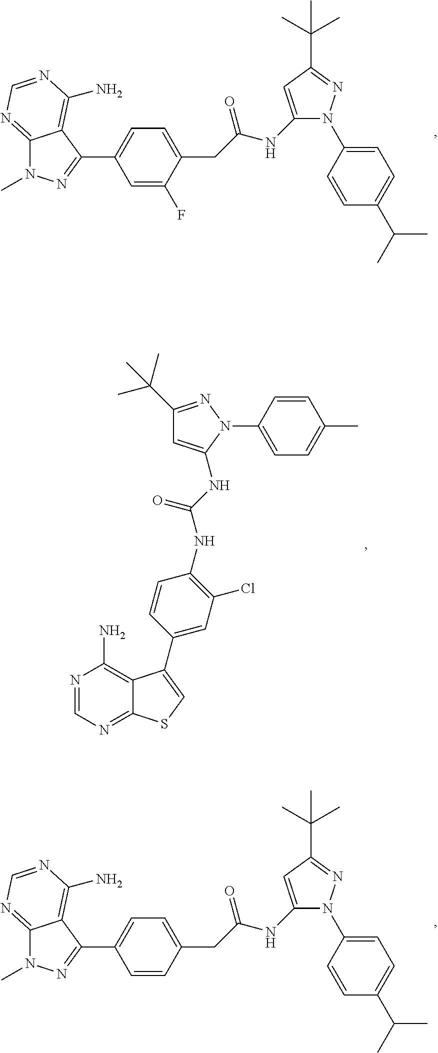

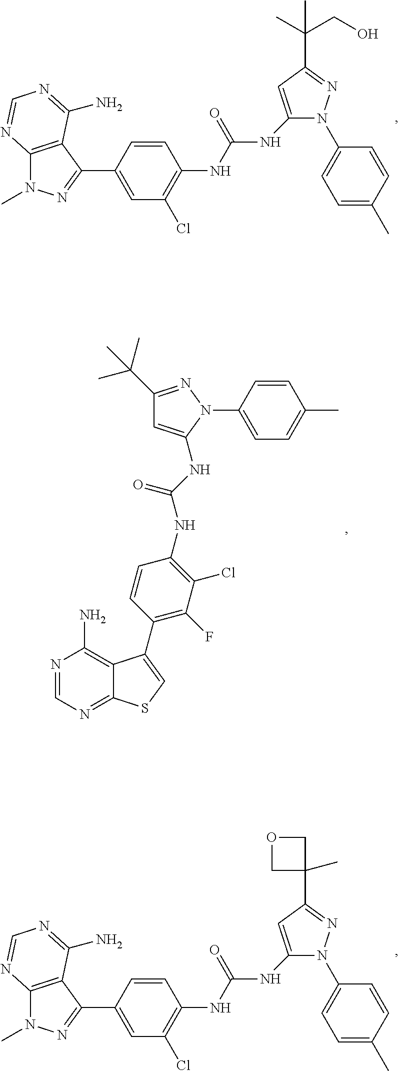

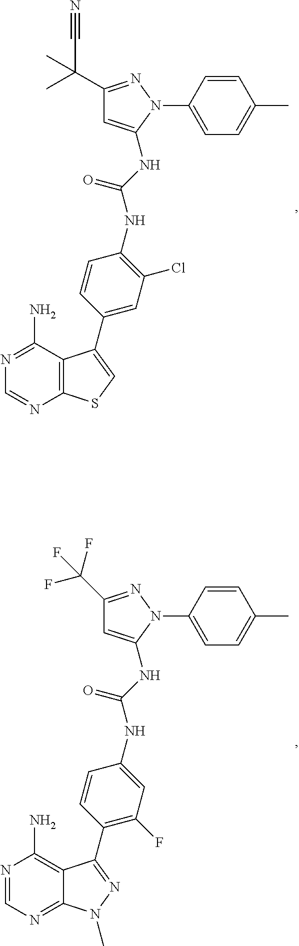 [SMILES: Cc1ccc(-n2nc(C(C)(C)C#N)cc2NC(=O)Nc2ccc(-c3csc4ncnc(N)c34)cc2Cl)cc1 ; Cc1ccc(-n2nc(C(F)(F)F)cc2NC(=O)Nc2ccc(-c3nn(C)c4ncnc(N)c34)c(F)c2)cc1]

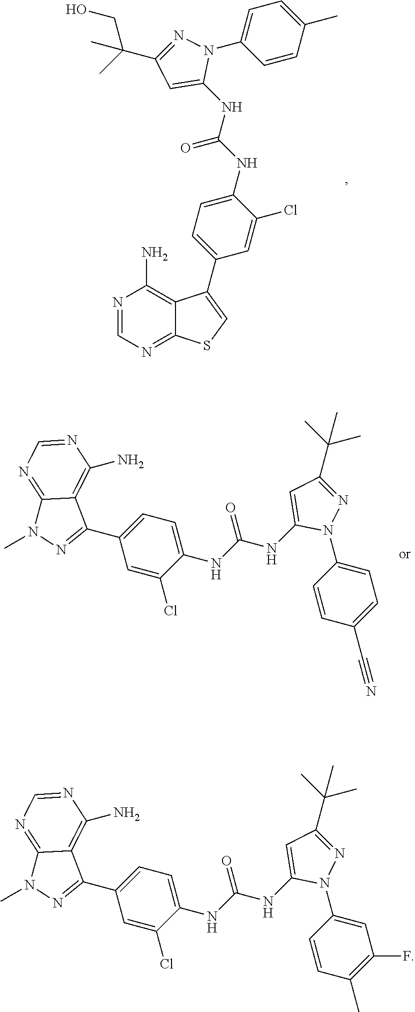

231-232. (canceled)

233. The composition of claim 116, wherein the NEK7 small molecule inhibitor compound is in contact with at least one NEK7 protein in a type 2 binding mode.

234. (canceled)
